(19) United States
(12) Patent Application Publication
(10) Pub. No.: US 2004/0133356 A1
(43) Pub. Date: Jul. 8, 2004

(54) Three-dimensional model of a Fc region of an IgE antibody and uses thereof

(76) Inventors: Theodore S. Jardetzky, Chicago, IL (US); Beth A. Wurzburg, Evanston, IL (US)

Correspondence Address: HESKA CORPORATION, INTELLECTUAL PROPERTY DEPT., 1613 PROSPECT PARKWAY, FORT COLLINS, CO, 80525, US

(21) Appl. No.: 10/704,406

(22) Filed: Nov. 7, 2003

Related U.S. Application Data
(62) Division of application No. 09/809,746, filed on Mar. 15, 2001, now abandoned.
(60) Provisional application No. 60/234,877, filed on Sep. 22, 2000.
(60) Provisional application No. 60/189,403, filed on Mar. 15, 2000.

(51) Int. Cl. G06F 19/00; G01N 33/48; G01N 33/50

(52) U.S. Cl. 702/19

(57) ABSTRACT

The present invention includes three-dimensional models of antibodies, such as Fc-Cε3/Cε4 regions of IgE antibodies, as well as methods to produce such models. The present invention also includes muteins having increased stability and/or antibody receptor binding activity, as well as methods to produce such muteins, preferably using information derived from three-dimensional models of the present invention. Also included are nucleic acid sequences encoding muteins of the present invention and use of those sequences to produce such muteins. Also included is the use of the model to identify compounds that inhibit the binding of an antibody receptor protein to an antibody. The present invention also includes uses of such muteins and inhibitory compounds, for example, in methods to diagnose and protect animals from allergy and other abnormal immune responses.

### CROSS-REFERENCE TO RELATED APPLICATIONS

This Application claims priority to provisional application U.S. Ser. No. 60/189,403, filed Mar. 15, 2000, entitled THREE-DIMENSIONAL MODEL OF A Fc REGION OF AN IgE ANTIBODY AND USES THEREOF; and provisional application U.S. Ser. No. 60/234,877, filed Sep. 22, 2000, entitled “STRUCTURE OF THE HUMAN IgE-Fc Cε3-Cε4 REVEALS CONFORMATIONAL FLEXIBILITY IN THE ANTIBODY EFFECTOR DOMAINS.”

This invention was made at least in part with government support under NIH Grant No. RO1 AI38972, awarded by the National Institutes of Health to Northwestern University. The government has certain rights to this invention.

### FIELD OF THE INVENTION

The present invention relates to a crystal and a three-dimensional (3-D) model of a constant region of an IgE antibody that includes the Cε3 and Cε4 domains (Fc-Cε3/Cε4, or Fc-Ce3/Ce4, region). The present invention also relates to the use of that model to produce muteins and inhibitors useful in the diagnosis and treatment of allergy and the regulation of other immune responses in an animal.

### BACKGROUND OF THE INVENTION

Antibody Fc-receptors (FcRs) play an important role in the immune response by coupling the specificity of secreted antibodies to a variety of cells of the immune system. A number of cell types, including macrophages, mast cells, eosinophils, and basophils, express membrane-bound FcRs at their surfaces. The binding of antibodies to FcRs provides antigen-specificity to these cells, which upon activation release further cell-specific mediators of the immune response, such as interleukins, initiators of inflammation, leukotrienes, prostaglandins, histamines, or cytotoxic proteins. The adoptive specificity of the FcRs allows a combinatorial approach to pathogen elimination, by coupling the diversity of antibody antigen-recognition sites to the variety of cell-types expressing these receptors.

FcR-initiated mechanisms are important in normal immunity to infectious disease as well as in allergies, antibody-mediated tumor recognition, autoimmune diseases, and other diseases in which immune responses are abnormal (i.e., not regulated). Recent experiments with transgenic mice have demonstrated that the FcRs control key steps in the immune response, including antibody-directed cellular cytotoxicity and inflammatory cascades associated with the formation of immune complexes; see, for example, Ravetch et al., 1998, Annu Rev Immunolo 16, 421-432. Receptors that bind IgG (FcgRI, FcgRII, and FcgRIII, known collectively as FcgRs) mediate a variety of inflammatory reactions, regulate B-cell activation, and also trigger hypersensitivity reactions. The high affinity Fc epsilon receptor (also known as the IgE receptor or FceRI) is associated with the activation of mast cells and the triggering of allergic reactions and anaphylactic shock. Knockout mice for the FceRI alpha chain (FcεRIα) are unable to mount IgE-mediated anaphylaxis (see for example, Dombrowicz et al., 1993, Cell 75, 969-976), although FcgRs are still able to activate mast cells (see, for example, Dombrowicz et al., 1997, J. Clin. Invest. 99, 915-925; Oettgen et al., 1994, Nature 370, 367-370). FceRI has also been shown to trigger anti-parasitic reactions from platelets and eosinophils as well as deliver antigen into the MHC class II presentation pathway for the activation of T cells; see, for example, Gounni et al., 1994, Nature 367, 183-186; Joseph et al., 1997, Eur. J. Immunol. 27, 2212-2218; Maurer et al., 1998, J. Immunol. 161, 2731-2739. The beta subunit of FceRI has been associated with asthma in genetic studies; see, for example, Hill et al., 1996, Hum. Mol. Genet. 5, 959-962; Hill et al., 1995, Bmj 311, 776-779; Kim et al., 1998, Curr. Opin. Pulm. Med. 4, 46-48; Mao et al., 1998, Clin. Genet. 53, 54-56; Shirakawa et al., 1994, Nat. Genet. 7, 125-129. A significant fraction of the population (˜20%) may be affected by allergies, and this century has seen a substantial increase in asthma. Since IgE binding to FceRI is a requisite event in the reaction to different allergens, therapeutic strategies aimed at inhibiting FceRI could provide a useful treatment for these diseases. For example, monoclonal antibodies that target IgE and block receptor binding have shown therapeutic potential; see, for example, Heusser et al., 1997, Curr. Opin. Immunol. 9, 805-813.

FceRI is found as a tetrameric (abg₂) or trimeric (ag₂) membrane bound receptor on the surface of mast cells, basophils, eosinophils, langerhans cells and platelets. The alpha chain, also referred to as FcεRIα, of FceRI binds IgE molecules with high affinity (K_{D} of about 10⁻⁹ to 10⁻¹⁰ moles/liter (M)), and can be secreted as a 172-amino acid soluble, IgE-binding fragment by the introduction of a stop codon before the single C-terminal transmembrane anchor; see, for example, Blank et al.,1991,E. J. Biol. Chem. 266, 2639-2646, which describes the secretion of a soluble IgE-binding fragment of 172 amino acids. The extracellular domains of the human FcεRIα protein belong to the immunoglobulin (Ig) superfamily and contain seven N-linked glycosylation sites. Glycosylation of FcεRIα affects the secretion and stability of the receptor, but is not required for IgE-binding; see, for example, LaCroix et al., 1993, Mol. Immunol. 30, 321-330; Letoumeur et al.,1995, J. Biol. Chem. 270, 8249-8256; Robertson, 1993, J. Biol. Chem. 268, 12736-12743; Scarselli et al., 1993, FEBS Lett 329, 223-226. The beta and gamma chains of FceRI are signal transduction modules.

Prior investigators have disclosed the nucleic acid sequence for human FcεRIα; see, for example, U.S. Pat. No. 4,962,035, by Leder, issued Oct. 9, 1990; U.S. Pat. No. 5,639,660, by Kinet et al., issued Jun, 17, 1997; Kochan et al., 1988, Nucleic Acids Res. 16, 3584; Shimizu et al., 1988, Proc. Natl. Acad. Sci. USA 85, 1907-1911; and Pang et al., 1993, J. Immunol. 151, 6166-6174. Nucleic acid sequences have also been reported for the human FceRI beta and gamma chains; see, respectively, Kuster et al., 1992, J. Biol. Chem. 267, 12782-12787; Kuster et al., 1990, J. Biol. Chem. 265, 6448-6452. Nucleic acid sequences have also been reported for nucleic acid molecules encoding canine FcεRIα, murine FcεRIα, rat FcεRIα, feline FcεRIα and equine FcεRIα proteins; see, respectively, GenBank™ accession number D16413; Swiss-Prot accession number P20489 (represents encoded protein sequence); GenBank accession number J03606; PCT Publication No. WO 98/27208, by Frank et al., published Jun. 25, 1998, referred to herein as WO 98/27208; and PCT Publication No. WO 99/38974, by Weber et al., published Aug. 5, 1999, referred to herein as WO 99/38974. In addition, methods to detect IgE antibodies using a FcεRIα protein have been reported in PCT Publication No. WO 98/23964, by Frank et al., published Jun. 4, 1998, referred to herein as WO 98/23964; WO 98/27208, ibid.; PCT Publication No. WO 98/45707, by Frank et al., published Oct. 15, 1998, referred to herein as WO 98/45707; and WO 99/38974, ibid.. WO 98/23964, WO 98/27208, WO 98/45707 and WO 99/38974 are each incorporated by reference herein in its entirety.

There have been several reports of the use of mutagenesis and swapping techniques to attempt to identify amino acids of either FcεRIα or IgE involved in the binding of (i.e., interaction between) those respective proteins, reports attempting to model FcεRIα proteins based on homology to other Ig-superfamily members, and reports that identify compounds that apparently inhibit such binding; see, for example, Cook et al., 1997, Biochemistry 36, 15579-15588; Hulett et al., 1994, J. Biol. Chem. 269, 15287-15293; Hulett et al., 1995, J. Biol. Chem 270, 21188-21194; Mallamaci et al., 1993, J. Biol. Chem. 268, 22076-22083; Robertson, 1993, ibid.; Scarselli et al., 1993, ibid. McDonnell et al., 1997, Biochem. Soc. Trans. 25, 387-392; McDonnell et al., 1996, Nat. Struc. Biol. 3, 419-426; PCT Publication No. WO 97/40033, by Cheng et al., published Oct. 30, 1997; U.S. Pat. No. 5,180,805, by Gould et al, issued Jan. 19, 1993; U.S. Pat. No. 5,693,758, by Gould et al., issued Dec. 2, 1997; PCT Publication No. WO 96/01643, by Gould et al., published Jan. 25, 1996; PCT Publication No. WO 95/14779, by Gould et al., published Jun. 1, 1995. None of these references, however, describe isolated crystals of FcεRIα proteins or 3-D models derived from crystals.

Despite what is known about FcRs and their interaction with antibodies, there remains a need for FcRs and antibodies with improved characteristics, such as enhanced affinity for their ligands, altered substrate specificity, increased stability, and increased solubility for use in diagnosis, treatment and prevention of allergy and other abnormal immune responses. Also needed for safe and efficacious compounds to prevent or treat allergy and to regulate other immune responses in an animal.

### SUMMARY OF THE INVENTION

The present invention includes an isolated crystal of a constant region (Fc region) of an antibody, a three-dimensional (3-D) model of such a crystal and a modification of such a model. The present invention also includes compounds that inhibit the ability of FcRs to bind to antibodies as well as antibody muteins and other modified antibodies. Also included in the present invention are methods to produce and use such crystals, models, inhibitory compounds, muteins, and other modified proteins. As such, the present invention includes antibodies with improved functions such as increased stability, increased affinity for an Ig binding domain of a FcR, altered substrate specificity, and increased solubility, including but not limited to reduced aggregation. Such proteins, also referred to as muteins, are useful to detect allergy and other immune response abnormalities as well as to protect an animal from such abnormalities. The present invention also provides safe and efficacious inhibitory compounds to protect (e.g., prevent, treat, reduce the consequences of) an animal from allergy and to regulate other immune responses in an animal.

The present invention includes a 3-D model of a human IgE Fc region comprising Cε3 and Cε4 domains, wherein the model substantially represents the atomic coordinates specified in Table 1, Table 2 or Table 3. The present invention also includes a 3-D model comprising a modification of a model substantially representing the atomic coordinates specified in Table 1, Table 2 or Table 3. Also included in the present invention are methods to produce such models.

The present invention also includes an isolated crystal of a human IgE Fc region comprising Cε3 and Cε4 domains.

The present invention includes a method to identify a compound that inhibits the binding between an IgE antibody and a FcεRIα protein. The method includes the step of using a 3-D model of the present invention, and particularly one substantially represents the atomic coordinates specified in Table 1, Table 2 or Table 3. Also included in the present invention are inhibitory compounds identified using such a method. Also included are therapeutic compositions that include such inhibitory compounds and methods to use such therapeutic compositions to protect an animal from allergy or to regulate other immune responses (e.g., protect an animal from other abnormal immune responses).

The present invention also includes a mutein that binds to a Fc binding domain of a FcR. Such a mutein has an improved function compared to a protein that includes SEQ ID NO:2. Examples of such an improved function include increased stability, increased affinity for an Fc domain of an antibody, altered substrate specificity, decreased aggregation, and increased solubility. Such a mutein is produced by a method that includes the following steps: (a) analyzing a 3-D model substantially representing the atomic coordinates specified in Table 1, Table 2, or Table 3 to identify at least one amino acid of the protein represented by the model which if replaced by a specified amino acid would effect an improved function of the protein; and (b) replacing the identified amino acid(s) to produce the mutein having such an improved function. The present invention also includes a mutein having an improved function compared to an unmodified IgE Fc region.

Also included are muteins that are chemically modified IgE Fc regions. Also included are nucleic acid molecules that encode muteins of the present invention, recombinant molecules and recombinant cells including such nucleic acid molecules and methods to produce such muteins. Also included are diagnostic reagents and diagnostic kits including such muteins, therapeutic compositions including such muteins, and methods to detect or protect an animal from allergy or other abnormal immune responses.

The present invention also includes a method to improve a function of a IgE Fc region which includes the steps of: (a) analyzing a 3-D model substantially representing the atomic coordinates specified in Table 1, Table 2 or Table 3 to identify at least one amino acid of the protein which if replaced by a specified amino acid improves at least one of the functions of the protein; and (b) replacing the identified amino acid(s) to produce a mutein having at least one of the improved functions.

### BRIEF DESCRIPTION OF DRAWINGS

FIG. 1 shows a side-view comparison of the unbound IgE-Fc, receptor-bound IgE-Fc and IgG-Fc structures. The N-terminal domains are shown in blue. FIG. 1a shows the closed form of IgE-Fc Cε3-Cε4 domains. FIG. 1b shows the open form of IgE-Fc Cε3-Cε4 domains. FIG. 1c shows unbound IgG-Fc.

FIG. 2 is a top-view comparison of the unbound IgE-Fc, receptor-bound IgE-Fc and IgG-Fc structures (N-terminal domains). β-strands are labeled (A-G) and a line is drawn between the first residue of the A strands for each Fc structure. In the closed IgE confirmation, this distance is 13 Å, in the open form it is 23 Å and in the IgG-Fc structure it is 22 Å. FIG. 2a shows the closed form of IgE-Fc Cε3-Cε4 domains. FIG. 2b shows the open form of IgE-Fc Cε3-Cε4 domains. FIG. 2c shows unbound IgG-Fc.

FIG. 3 shows a superposition of nine crystallographically independent IgG-Fc structures (grey/blue) with the open (dark blue) and closed (red) IgE-Fc structures. The IgG and IgE Fc structures were superimposed using Cα carbons from the C-terminal domain (Cγ2 or Cε3). IgG-Fc structures were used from the PDB files 1IGT, 1FC1, 1FC2, 1FCC, 1IGY and 1ADQ. The 1MCO hinge-deleted antibody structure was not included in this analysis since it exhibits anomalous domain pairing throughout the protein structure. An asterisk is placed next to residue 366 in the BC loop of the IgE-Fc.

FIG. 4a shows a DynDom analysis of the domain motions characterizing the structural differences between bound and free IgE-Fc. One-half of an Fc (c3/c4 monomer) is shown in the closed conformation with the axis of the bend indicated by the dark red line. DynDom was used to determine the location of the axis and to calculate the change in the angle. Hinge residues (343-345, 351-352, and 435-436) are outlined in light purple. Residues that remain relatively fixed in both the open and closed forms of the Fc include the entire Cε4 domain, the interdomain linker, and the AB helix of Cε3. Residues in the Cε3 domain move as a semi-rigid domain. FIG. 4b shows a closeup of the residues at the Cε3/Cε4 and Cγ2/Cγ3 interfaces. IgG-Fc (red)(from pdb files 1IGT) and the closed IgE-Fc (blue) were superimposed using residues in the C-terminal domain (Cγ3 or Cε4). Note the displacement of the IgE-Fc helix (blue cylinder) away from the interdomain interface and the close approach of Cε3 residues to the IgG-Fc helix. The interactions of the AB helix with both domains may determine the full range of mobility of different antibody Fc domains. FIG. 4c shows a graph of the residue displacements observed for both chains of the IgE-Fc in the free and receptor-bound crystal structures. One chain is shown with red circles, the other with blue diamonds. The Cα distances between amino acid residues in each structure was calculated after superposition of the two structures based on the alignment of the two Cε4 domains. Loops involved in binding receptor are indicated and highlighted in yellow and move by approximately 6-14 Å in the free form. “N” indicates Cε3 A strand residues, “C” is the carboxy terminus, “L” identifies the poorly ordered Cε4 AB loop, and “X” identifies a difference due to crystal contacts.

FIG. 5a shows a surface representation of the Cε3 and Cε4 domains (top-view) in the closed (left) and open (right) IgE-Fc structures. Receptor binding residues are shown in magenta and are from the Cε3 BC, DE and FG loops. FIG. 5b shows a side-view of the Cε3 and Cε4 domains described in FIG. 5a.

FIG. 6 illustrates potential roles for IgE conformational changes in receptor-binding and structure-based inhibitor design. Open forms of the IgE molecule can interact with the high-affinity receptor (FcεRI), as shown by the crystal structure of the complex. IgE also binds to a low-affinity receptor, which is a trimer of C-type lectin domains (FceRII). FceRII could potentially interact with the closed form of the IgE structure as shown in the upper left hand portion of the figure. The lectin domains are shown in blue while the IgE-Fc Cε3 domains are shown in yellow. Only two of the three lectin domains are thought to interact with the IgE. Three distinct classes of inhibitors of the IgE interaction are also shown on the left hand side of the figure. One class could bind to the open IgE and compete for FceRI-binding sites (competitive, lower left). Another class could bind to a region of IgE near the Ce3/Ce4 hinge and stabilize the closed form of IgE, thus inhibiting FceRI binding indirectly (allosteric inhibitor, center left). A third class of inhibitors could interact with FceRI-binding regions of the IgE, but stabilize a closed form of the IgE, acting as both a competitive and conformational inhibitor of FceRI binding (conformational inhibitor, center left).

FIG. 7a depicts the general structure of the IgG and IgE antibodies. Both antibodies contain two isotype-specific heavy chains and two light chains (H₂L₂). The Fab domains contain both heavy and light chain components while the Fc domains (shaded pink) are derived exclusively from the light chains. The IgE-Fc contains an extra domain pair (Cε2) compared to the IgG-Fc. The IgE Cε3-Cε4 domains are homologous to the IgG Cγ2-Cγ3 domains. FIG. 7b shows a structure-based sequence alignment of human IgE-Fc Cε3-Cε4 with the sequences of four IgG-Fc's for which crystal structure have been solved. IgE secondary structure is indicted using arrows for β-strands and ribbons for α-helices. Color bars indicate hinge residues (blue), FcεRi-binding loops (pink) and carbohydrate attachment sites (green dots). Within the sequence alignment, conserved residues are indicated with light-blue shading while structural differences (insertions, deletions, changes in secondary structure) between the IgG and IgE are highlighted in yellow. In addition, the completely conserved Cγ2 AB helix histidine residue (H310 in Igg1, H329 in IgG2a) and the corresponding residue in IgE, threonine 409, are indicated in yellow and pink respectively. The IgE numbering (above the sequence) is according to Dorrington and Bennich. The numbering of human IgG1 is given directly below the sequence. The PDB numbering of murine IG2a (1IGT), is shown in italics at the bottom (note that there are deletions in this numbering system).

FIG. 8a shows a side-view comparison of the unbound IgE-Fc, receptor-bound IgE-Fc and IgG-Fc structures. The N-terminal domains are shown in blue. FIG. 8b is a top-view comparison of the unbound IgE-Fc, receptor-bound IgE-Fc and IgG-Fc structures (N-terminal domains). β-strands are labeled (A-G) and a line is drawn between the first residue of the A strands for each Fc structure. In the closed IgE confirmation, this distance is 13 Å, in the open form it is 23 Å and in the IgG-Fc structure it is 22 Å.

FIG. 9a shows a superposition of nine crystallographically independent IgG-Fc structures (grey/blue) with the open (dark blue) and closed (red) IgE-Fc structures. The IgG and IgE Fc structures were superimposed using Cα carbons from the C-terminal domain (Cγ2 or Cε3). IgG-Fc structures were used from the PDB files 1IGT, 1FC1, 1 FC2, 1FCC, 1IGY and 1ADQ. An asterisk is placed next to residue 366 in the BC loop of the IgE-Fc. Note the displacement of the IgE-Fc helix away from the interdomain interface, the movement of the IgE-Fc EF helix in the closed conformation, and the close approach of the IgG-Fc AB and EF helices at the site of the IgG residue insertion. FIG. 9b shows a DymDom analysis of the IgG-Fc. A stereo view of one chain of the Fc (closed conformation) is shown with the rotation axis indicated by an arrow. Hinge residues (343-345, 351-352, and 435-436) are outlined in cyan. Cε3 domain residues that move as a semi-rigid domain are shown in red. Residues that remain relatively fixed in both the open and closed forms of the Fc are shown in blue. FIG. 9c shows a DynDom analysis of three IgG-Fc structures. Shown is a stereo view with the rotation axes and hinge residues for murine IgG1 (1IGY) (cyan), murine IgG2a (1IGT) (purple) and human IgG1 (1FC1) (pink) on the Cα trace of the IgG2a structure. FIG. 9d shows the change in Cα coordinates between the closed and open conformations of the IgE-Fc. One chain is shown with red circles, the other with blue diamonds. Receptor binding loops are indicated and highlighted in pink; hinge residues are shown in cyan. “N” indicates Cε3 A strand residues, “C” is the carboxy terminus, “L” identifies the poorly ordered Cε4 AB loop, and “X” identifies a difference due to crystal contacts.

FIG. 10a diagrams the contacts made by the AB helix residues 9IgE Cε3 or IgG Cγ2). Residues of the AB and EF helices are shown on the grey helical wheels while the residues of the lower domain (Cε3 or Cε4) are shown below (blue lettering in blue ovals). Upper domain contacts (to Cε3 or Cγ2) involve residues in the EF helix and residues immediately adjacent to the AB helix. Lower domain contacts (to Cε4 or Cγ3) involve residues from the C, C′, F and G β-sheet strands and the FG loop. Contacts formed only in the open form of the IgE-Fc are indicated by dashed blue lines; the single contact formed only in the closed form is indicated by a red line. Contacts made by the conserved H329 in IgG are indicated by solid blue lines. The completely conserved EF helix H329 residue and the insertion residue I266 that forms a bulge just after the AB helix, are shown in yellow. FIG. 10b shows a surface representation of the interaction of EF helix residue T407 with the AB helix in the closed IgE-Fc while FIG. 10c shows this same interaction in the open IgE-Fc. FIG. 10d shows a surface representation of the packing interactions of the corresponding residue, the conserved h329, in IgG-Fc, with the bulge a the C-terminus of the Cγ2 AB helix.

FIG. 11a shows a molecular surface representation of the Cε3 and Cε4 domains (side-view) in the closed (left) and open (right) IgE-Fc structures. Receptor binding residues are shown in magenta and are from the Cε3 BC, DE and FG loops. FIG. 11b shows a top-view of the Cε3 and Cε4 domains described in FIG. 11a.

FIG. 12 shows possible roles for IgE flexibility in Fc receptor binding and structure-based inhibitor design. The Cε3 domains are colored to correspond to the different conformational states, open (pink) and closed (yellow); the Cε4 domains are shown in grey. Open forms of the IgE molecule can bind to the high affinity receptor, FcεRI. The low affinity receptor, FcεRII, is a trimeric C-type lectin that binds to an unidentified conformation of the IgE-Fc (green). Three potential classes of inhibitors of the IgE:FcεRI interactions are shown: binding site competitive inhibitor, binding sire conformational inhibitor, and allosteric conformational inhibitor.

FIG. 13 illustrates a potential drug binding site near the IgE-Fc hinge. A hypothetical drug (green) is shown inside the hinge cavity. Residues surrounding the cavity include R342, P343, S344, P3435, L348, W410, I411, K435, T436, R440,P471, E472, D473,E529.

### DETAILED DESCRIPTION OF THE INVENTION

The present invention includes isolated crystals of Fc regions of antibodies, 3-D models of such crystals and modifications of such models. The present invention also includes compounds that inhibit the ability of FcRs to bind to antibodies as well as muteins and other modified antibodies. Also included in the present invention are methods to produce and use such crystals, models, inhibitory compounds, muteins, and other modified proteins.

The present invention includes an isolated crystal of a Fc region comprising the Cε3 and Cε4 domains of an IgE antibody (Fc-Cε3/Cε4), a 3-D model of such a crystal and a modification of such a model. As used herein, the term “a” entity or “an” entity refers to one or more of that entity; for example, a crystal or a model refers to one or more crystals or models, respectively. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures, or combinations, of two or more of the compounds.

As used herein, an extracellular domain of a FcεRIα protein is the portion of the FceRI alpha chain that is exposed to the environment outside the cell and that binds to the Fc domain of an IgE antibody. Such an extracellular domain can be (a) a complete extracellular domain which is a domain that extends from the first amino acid of a mature FceRI alpha chain through the last amino acid prior to the start of the transmembrane region or a domain that is functionally equivalent, in that such a domain includes a D1 and D2 domain, displays a similar affinity for the IgE antibody to which such an FcεRIα protein naturally binds, and produces crystals having sufficient quality to enable structure determination, or (b) a fragment of any of the extracellular domains of (a), wherein the fragment retains its ability to bind to the Fc domain of an antibody. As used herein, the terms binding to an antibody and binding to the Fc domain (i.e., constant region) of an antibody can be used interchangeably since it is recognized that a FcR binds to the Fc domain of an antibody. A FcR (i.e., a protein that can bind to an antibody), such as a FcεRIα protein, can be a full-length FcR (e.g., a full-length FceRI alpha chain), or any fragment thereof, wherein the fragment binds to an antibody. Similarly an antibody, or an Fc region thereof, can be a full-length antibody, or full-length Fc region thereof, or any fragment thereof that binds to a FcR. In one embodiment an Fc region comprises Cε3 and Cε4 domains. Preferably a FcR binds to an antibody with an affinity (K_{A}) of at least about 10⁸ liters/mole (M⁻¹), more preferably of at least about 10⁹ M⁻¹, and even more preferably of at least about 10¹⁰ M⁻¹.

The present invention is surprising in several aspects. For example, this is the first report of an isolated crystal of a Fc-Cε3/Cε4 region of an IgE antibody, and in particular of an isolated crystal of sufficient quality that a crystal structure, i.e., a 3-D model, could be derived therefrom. Generation of such a crystal was very difficult and non-obvious and has been attempted by others without success. The inventors tried many approaches before discovering a preferred Fc-Cε3/Cε4 region from which to make a useful crystal. The first such region to be used successfully is referred to herein as PhFc-Cε3/Cε4₁₋₂₂₂ which is composed of the four amino acids alanine, aspartic acid, proline and cysteine at the amino terminus followed by amino acids 330 through 547 of the human IgE Fc constant region, using the numbering system of Dorrington et al, 1978, Immunol Rev 41, 3-25. PhFc-Cε3/Cε4₁₋₂₂₂ is represented herein by SEQ ID NO:2. An example of a nucleic acid molecule encoding PhFc-Cε3/Cε4₁₋₂₂₂ is referred to herein as nhFc-Cε3/Cε4₁₋₆₆₆, the nucleic acid sequence of which is referred to herein as SEQ ID NO: 1. It was also discovered that better crystals are generated when PhFc-Cε3/Cε4₁₋₂₂₂ is produced in insect cells, using a method such as that described in the Examples. Solution of the crystal structure was also very difficult, as described in more detail in the Examples. For example, as part of the effort, approximately 12,000 models were generated and used in complete Molecular Replacement searches with the program Amore, taking about 10 days on 5 Silicon Graphics computers.

Determination of the crystal structure of PhFc-Cε3/Cε4₁₋₂₂₂ produced in Trichoplusia ni (Hi-5) cells resulted in a 3-D model that substantially represents the atomic coordinates specified in Table 1, Table 2 or Table 3. Amino acids are represented herein by their standard three or one letter codes; see, for example, Sambrook et al., Molecular Cloning: A Laboratory Manual, Cold Spring Harbor Labs Press, 1989, which is incorporated herein by reference in its entirety.

The 3-D model of PhFc-Cε3/Cε4₁₋₂₂₂ is also very surprising in view of what is known about the crystal structure of the Fc region of IgG. The Fc region of IgE exists in a novel conformation that is more compact than that of IgG. The Cε3 domains are also much closer to each other in IgE compared to IgG (about 13 angstroms compared to about 22 angstroms), leading to the descriptor of “closed conformation” for the IgE Fc structure. This closed conformation is also surprising in view of the crystal structures of FcεRIα alone, which is disclosed in U.S. patent application Ser. No. 09/434,193, filed Nov. 4, 1999, by Jardetzky et al., and in PCT Publication No. WO 00/26246, published May 11, 2000, by Jardetzky et al., and of the complex between FcεRIα and Fc-Cε3/Cε4 alone, which is disclosed in U.S. patent application Ser. No. 60/189,853. U.S. Ser. No. 09/434,193, ibid., WO 00/26246, ibid. and 60/189,853, ibid., are incorporated by reference herein in their entireties. The structure of Fc-Cε3/Cε4 in the complex is an open conformation, also referred to as a receptor-bound conformation. The distance between the two Cε3 domains in the receptor-bound conformation is about 23 angstroms. Comparison of these structural similarities and differences are described in greater detail in the Examples and in Ser. No. 60/189,853, ibid. Analysis of the model which substantially represents the atomic coordinates specified in Table 1, Table 2 or Table 3 indicates the necessity of such a model for proper interpretation and refinement of mutagenesis studies that have been reported. Such a model permits differentiation between amino acids directly or indirectly influencing binding of FcεRIα to IgE and demonstrates where amino acids and amino acid segments identified in mutagenesis studies are positioned on the protein. By using a model of the present invention one can identify the interactions of FcεRIα and IgE, thereby identifying amino acids to target for mutein production or regions to target for the development of compounds to inhibit binding of IgE to its receptor. Such a model also leads to the ability to design inhibitory compounds that stabilize the closed conformation of IgE, thereby reducing its ability to bind to a FcR. Such a model can be used alone or in conjunction with a model of FcεRIα alone (U.S. Ser. No. 09/434,193, ibid. and WO 00/26246, ibid.) or of the complex between FcεRIα and Fc-Cε3/Cε4 alone (Ser. No. 60/189,853, ibid.).

One embodiment of the present invention is an isolated crystal of a Fc-Cε3/Cε4 region of an IgE antibody. As used herein, an isolated crystal is a crystal of a protein that has been produced in a laboratory; that is, an isolated crystal is produced by an individual and is not an object found in situ in nature. It is appreciated by those skilled in the art that there are a variety of techniques to produce crystals including, but not limited to, vapor diffusion using a hanging or sitting drop methodology, vapor diffusion under oil, and batch methods; see, for example, Ducruix et al., eds., 1991, Crystallization of nucleic acids and proteins; A practical approach, Oxford University Press, and Wyckoff et al., eds., 1985, Methods in Enzymology 11, 49-185; each reference is incorporated by reference herein in its entirety. It is also to be appreciated that crystallization conditions can be adjusted depending on a protein's inherent characteristics as well as on a protein's concentration in a solution and that a variety of precipitants can be added to a protein solution in order to effect crystallization; such precipitants are known to those skilled in the art. In a preferred embodiment, a crystal of a Fc-Cε3/Cε4 region is produced in a solution by adding a precipitant such as polyethylene glycol (PEG) or PEG monomethylether. It is also to be noted that a Fc-Cε3/CCε4 region used to produce a crystal can be produced by a variety of methods, including purification of a native protein, chemical synthesis of a protein, or recombinant production of a protein. Although a number of cell types can be used to recombinantly produce such a protein, insect cells, such as, but not limited to Trichoplusia ni and Spodoptera frugiperda, are preferred, with Trichoplusia ni cells being more preferred. Additional methods to produce proteins are disclosed below.

Isolated crystals of the present invention can include heavy atom derivatives, such as, but not limited to, gold, platinum, mercury, selenium, copper, and lead. Such heavy atoms can be introduced randomly or introduced in a manner based on knowledge of 3-D models of the present invention. Additional crystals of the present invention are not derivatized. In one embodiment, an isolated crystal of the present invention is a co-crystal of a FcεRIα protein bound to a Fc domain of an IgE antibody in the presence of a compound that inhibits the binding of a FcεRIα protein to a Fc domain of an IgE antibody. Additional crystals of the present invention include crystals produced from proteins that are muteins of the present invention or other proteins that are represented by a 3-D model of the present invention.

An isolated crystal of the present invention can be the crystal of any suitable Fc region that binds to FcεRIα, such as a Fc comprising Cε3 domains or a Fc comprising Cε3 and Cε4 domains. Suitable Fc-Cε3/Cε4 regions include mammalian Fc-Cε3/Cε4 regions, with human, canine, feline, equine, rat and murine Fc-Cε3/Cε4 regions being preferred, and human Fc-Cε3/Cε4 regions being even more preferred. A preferred crystal of the present invention diffracts X-rays to a resolution of about 4.5 angstroms or higher (i.e., lower number meaning higher resolution), with resolutions of about 4.0 angstroms or higher, about 3.5 angstroms or higher, about 3.25 angstroms or higher, about 3 angstroms or higher, about 2.5 angstroms or higher, about 2.3 angstroms or higher, about 2 angstroms or higher, about 1.5 angstroms or higher, and about 1 angstrom or higher being increasingly more preferred. It is appreciated, however, that additional crystals of lower resolutions can have utility in discerning overall topology of the structures, e.g., location of a binding site or where a molecule binds to a receptor or to an antibody. A particularly preferred isolated crystal of the present invention has amino acid sequence SEQ ID NO:2, or a sequence essentially equivalent that represents another mammalian Fc-Cε3/Cε4 region. Preferred are crystals that belong to spacegroup P42₁2. Particularly preferred crystals include a crystal belonging to spacegroup P42₁2 that has cell dimensions of 105.6 angstroms×105.6 angstroms×47.1 angstroms, alpha=beta=gamma=90 degrees and that contains one Cε3/Cε4 chain per asymmetric unit of the crystal. Such a preferred crystal preferably diffracts X-rays to a resolution of about 2.3 angstroms.

The present invention includes a 3-D model of a Fc-Cε3/Cε4 region that substantially represents the atomic coordinates specified in Table 1, Table 2 or Table 3. The present invention also includes 3-D models that comprise modifications of the model substantially represented by the atomic coordinates specified in Table 1, Table 2 or Table 3. Each such modification represents an antibody Fc region that binds to a Fc receptor protein. A 3-D model of a Fc-Cε3/Cε4 region is a representation, or image, that predicts the actual structure of the corresponding region. As such, a 3-D model is a tool that can be used to probe the relationship between the region's structure and function at the atomic level and to design muteins (i.e., genetically and/or chemically altered antibodies) having an improved function, such as, but not limited to: increased (i.e., enhanced) stability; increased FcR binding activity, for example, by, increasing the affinity for an FcR by, for example, increasing the association rate and/or decreasing the dissociation rate between a FcR and an antibody or by altering substrate specificity (e.g., enhancing the ability of an Fc region of a certain species and class to bind to an antibody binding site from another species and/or another antibody class); and/or increased solubility (e.g., reduced aggregation). It is well known to those skilled in the art, however, that a 3-D model of a protein derived by analysis of protein crystals is not identical to the inherent structure of the protein. See, for example, Branden et al., Introduction to Protein Structure, Garland Publishing Inc., New York and London, 1991, especially on page 277, which states “not surprisingly the model never corresponds precisely to the actual crystal.” Furthermore, the model can be subjected to further refinements to more closely correspond to the actual structure of a Fc region of an antibody. Such a refined model, which is an example of a modification of the present invention, is a better predictor of the actual structure and mechanism of action of the protein that the model represents. A refinement of a 3-D model of the present invention refers to an improved model of a Fc-Cε3/Cε4 region that can be obtained in a variety of ways known to those skilled in the art. Refinements can include models determined to more preferred degrees of resolution, preferably to about 4.5 angstroms, more preferably to about 4 angstroms, more preferably to about 3.5 angstroms, more preferably to about 3.25 angstroms, more preferably to about 3 angstroms, more preferably to about 2.5 angstroms, more preferably to about 2.3 angstroms, more preferably to about 2 angstroms, more preferably to about 1.5 angstroms, and even more preferably to about 1 angstrom. Preferred refinements are obtained using the 3-D model as a basis for such improvements.

One embodiment of the present invention is a 3-D model of a Fc-Cε3/Cε4 region that substantially represents the atomic coordinates specified (i.e., listed) in Table 1.

TABLE 1
Atomic coordinates of 7_more_dimer.pdb

| ATOM | ATOM | | | | | | | | |
|---|---|---|---|---|---|---|---|---|---|
| # | TYPE | RES | CHN | # | X | Y | Z | OCC | B |
| 1 | CB | VAL | A | 336 | 46.217 | 60.546 | 16.604 | 1.00 | 63.58 |
| 2 | CG1 | VAL | A | 336 | 45.590 | 59.856 | 15.405 | 1.00 | 64.78 |
| 3 | CG2 | VAL | A | 336 | 47.677 | 60.888 | 16.335 | 1.00 | 68.51 |
| 4 | C | VAL | A | 336 | 44.045 | 61.436 | 17.434 | 1.00 | 61.62 |
| 5 | O | VAL | A | 336 | 43.880 | 60.399 | 18.074 | 1.00 | 64.02 |
| 6 | N | VAL | A | 336 | 46.161 | 62.624 | 17.972 | 1.00 | 59.58 |
| 7 | CA | VAL | A | 336 | 45.438 | 61.827 | 16.943 | 1.00 | 61.95 |
| 8 | N | SER | A | 337 | 43.046 | 62.262 | 17.137 | 1.00 | 59.54 |
| 9 | CA | SER | A | 337 | 41.679 | 61.982 | 17.564 | 1.00 | 57.94 |
| 10 | CB | SER | A | 337 | 41.204 | 63.069 | 18.525 | 1.00 | 56.70 |
| 11 | OG | SER | A | 337 | 41.581 | 64.347 | 18.053 | 1.00 | 65.28 |
| 12 | C | SER | A | 337 | 40.697 | 61.833 | 16.400 | 1.00 | 56.67 |
| 13 | O | SER | A | 337 | 40.987 | 62.234 | 15.268 | 1.00 | 56.61 |
| 14 | N | ALA | A | 338 | 39.539 | 61.245 | 16.687 | 1.00 | 52.92 |
| 15 | CA | ALA | A | 338 | 38.521 | 61.011 | 15.671 | 1.00 | 51.01 |
| 16 | CB | ALA | A | 338 | 38.576 | 59.562 | 15.216 | 1.00 | 50.96 |
| 17 | C | ALA | A | 338 | 37.120 | 61.348 | 16.166 | 1.00 | 50.95 |
| 18 | O | ALA | A | 338 | 36.816 | 61.204 | 17.352 | 1.00 | 53.16 |
| 19 | N | TYR | A | 339 | 36.272 | 61.805 | 15.250 | 1.00 | 47.68 |
| 20 | CA | TYR | A | 339 | 34.903 | 62.169 | 15.592 | 1.00 | 48.08 |
| 21 | CB | TYR | A | 339 | 34.810 | 63.675 | 15.897 | 1.00 | 50.34 |
| 22 | CC | TYR | A | 339 | 35.892 | 64.218 | 16.817 | 1.00 | 58.74 |
| 23 | CD1 | TYR | A | 339 | 37.196 | 64.421 | 16.355 | 1.00 | 60.22 |
| 24 | CE1 | TYR | A | 339 | 38.199 | 64.895 | 17.205 | 1.00 | 64.78 |
| 25 | CD2 | TYR | A | 339 | 35.616 | 64.506 | 18.153 | 1.00 | 60.49 |
| 26 | CE2 | TYR | A | 339 | 36.608 | 64.980 | 19.012 | 1.00 | 63.00 |
| 27 | CZ | TYR | A | 339 | 37.900 | 65.172 | 18.533 | 1.00 | 67.92 |
| 28 | OH | TYR | A | 339 | 38.891 | 65.626 | 19.382 | 1.00 | 68.47 |
| 29 | C | TYR | A | 339 | 33.954 | 61.815 | 14.437 | 1.00 | 45.49 |
| 30 | O | TYR | A | 339 | 34.262 | 62.044 | 13.267 | 1.00 | 43.52 |
| 31 | N | LEU | A | 340 | 32.808 | 61.238 | 14.775 | 1.00 | 44.01 |
| 32 | CA | LEU | A | 340 | 31.816 | 60.872 | 13.776 | 1.00 | 42.54 |
| 33 | CB | LEU | A | 340 | 31.550 | 59.363 | 13.816 | 1.00 | 39.07 |
| 34 | CG | LEU | A | 340 | 30.652 | 58.750 | 12.730 | 1.00 | 39.58 |
| 35 | CD1 | LEU | A | 340 | 31.198 | 59.074 | 11.349 | 1.00 | 28.76 |
| 36 | CD2 | LEU | A | 340 | 30.571 | 57.236 | 12.923 | 1.00 | 35.57 |
| 37 | C | LEU | A | 340 | 30.557 | 61.645 | 14.134 | 1.00 | 42.53 |
| 38 | O | LEU | A | 340 | 30.138 | 61.638 | 15.286 | 1.00 | 43.25 |
| 39 | N | SER | A | 341 | 29.957 | 62.324 | 13.160 | 1.00 | 42.42 |
| 40 | CA | SER | A | 341 | 28.752 | 63.103 | 13.425 | 1.00 | 40.32 |
| 41 | CB | SER | A | 341 | 28.997 | 64.586 | 13.105 | 1.00 | 41.30 |
| 42 | OG | SER | A | 341 | 29.416 | 64.758 | 11.764 | 1.00 | 45.86 |
| 43 | C | SER | A | 341 | 27.563 | 62.606 | 12.634 | 1.00 | 38.67 |
| 44 | O | SER | A | 341 | 27.707 | 62.048 | 11.559 | 1.00 | 42.94 |
| 45 | N | ARC | A | 342 | 26.378 | 62.823 | 13.177 | 1.00 | 37.60 |
| 46 | CA | ARC | A | 342 | 25.155 | 62.414 | 12.520 | 1.00 | 35.91 |
| 47 | CB | ARG | A | 342 | 24.026 | 62.359 | 13.552 | 1.00 | 36.67 |
| 48 | CG | ARG | A | 342 | 24.254 | 61.315 | 14.646 | 1.00 | 39.89 |
| 49 | CD | ARG | A | 342 | 23.141 | 61.322 | 15.679 | 1.00 | 42.31 |
| 50 | NE | ARG | A | 342 | 23.185 | 62.534 | 16.490 | 1.00 | 45.87 |
| 51 | CZ | ARC | A | 342 | 24.005 | 62.730 | 17.520 | 1.00 | 52.30 |
| 52 | NH1 | ARG | A | 342 | 24.861 | 61.790 | 17.895 | 1.00 | 56.74 |
| 53 | NH2 | ARG | A | 342 | 23.981 | 63.885 | 18.168 | 1.00 | 58.22 |
| 54 | C | ARC | A | 342 | 24.832 | 63.409 | 11.398 | 1.00 | 34.19 |
| 55 | O | ARC | A | 342 | 25.476 | 64.450 | 11.278 | 1.00 | 34.44 |
| 56 | N | PRO | A | 343 | 23.837 | 63.101 | 10.555 | 1.00 | 31.22 |
| 57 | CD | PRO | A | 343 | 23.028 | 61.871 | 10.494 | 1.00 | 34.08 |
| 58 | CA | PRO | A | 343 | 23.486 | 64.024 | 9.466 | 1.00 | 33.86 |
| 59 | CE | PRO | A | 343 | 22.437 | 63.244 | 8.663 | 1.00 | 32.10 |
| 60 | CO | PRO | A | 343 | 22.698 | 61.789 | 9.022 | 1.00 | 37.07 |
| 61 | C | PRO | A | 343 | 22.897 | 65.323 | 10.015 | 1.00 | 32.54 |
| 62 | O | PRO | A | 343 | 22.270 | 65.319 | 11.072 | 1.00 | 35.02 |
| 63 | N | SER | A | 344 | 23.090 | 66.432 | 9.311 | 1.00 | 33.04 |
| 64 | CA | SER | A | 344 | 22.515 | 67.687 | 9.778 | 1.00 | 34.74 |
| 65 | CE | SER | A | 344 | 23.172 | 68.897 | 9.095 | 1.00 | 35.18 |
| 66 | OG | SER | A | 344 | 22.663 | 69.093 | 7.792 | 1.00 | 35.70 |
| 67 | C | SER | A | 344 | 21.038 | 67.628 | 9.415 | 1.00 | 35.17 |
| 68 | O | SER | A | 344 | 20.669 | 67.132 | 8.349 | 1.00 | 32.53 |
| 69 | N | PRO | A | 345 | 20.162 | 68.094 | 10.313 | 1.00 | 35.98 |
| 70 | CD | PRO | A | 345 | 20.377 | 68.420 | 11.736 | 1.00 | 36.93 |
| 71 | CA | PRO | A | 345 | 18.731 | 68.056 | 9.995 | 1.00 | 34.79 |
| 72 | CE | PRO | A | 345 | 18.097 | 68.730 | 11.212 | 1.00 | 30.45 |
| 73 | CG | PRO | A | 345 | 18.988 | 68.221 | 12.331 | 1.00 | 30.91 |
| 74 | C | PRO | A | 345 | 18.385 | 68.739 | 8.667 | 1.00 | 34.13 |
| 75 | O | PRO | A | 345 | 17.471 | 68.317 | 7.963 | 1.00 | 34.14 |
| 76 | N | PHE | A | 346 | 19.124 | 69.783 | 8.309 | 1.00 | 31.57 |
| 77 | CA | PHE | A | 346 | 18.848 | 70.466 | 7.059 | 1.00 | 33.19 |
| 78 | CB | PHE | A | 346 | 19.711 | 71.724 | 6.935 | 1.00 | 37.49 |
| 79 | CG | PHE | A | 346 | 19.616 | 72.394 | 5.595 | 1.00 | 38.65 |
| 80 | CO1 | PHE | A | 346 | 18.432 | 72.979 | 5.178 | 1.00 | 42.62 |
| 81 | CD2 | PHE | A | 346 | 20.719 | 72.441 | 4.747 | 1.00 | 43.21 |
| 82 | CE1 | PHE | A | 346 | 18.344 | 73.608 | 3.933 | 1.00 | 41.72 |
| 83 | CE2 | PHE | A | 346 | 20.643 | 73.069 | 3.496 | 1.00 | 49.00 |
| 84 | CZ | PHE | A | 346 | 19.452 | 73.654 | 3.091 | 1.00 | 42.82 |
| 85 | C | PHE | A | 346 | 19.099 | 69.530 | 5.867 | 1.00 | 33.37 |
| 86 | O | PHE | A | 346 | 18.284 | 69.457 | 4.948 | 1.00 | 33.52 |
| 87 | N | ASP | A | 347 | 20.216 | 68.810 | 5.884 | 1.00 | 32.94 |
| 88 | CA | ASP | A | 347 | 20.533 | 67.891 | 4.792 | 1.00 | 33.61 |
| 89 | CE | ASP | A | 347 | 21.962 | 67.358 | 4.927 | 1.00 | 29.68 |
| 90 | CG | ASP | A | 347 | 23.011 | 68.378 | 4.541 | 1.00 | 35.85 |
| 91 | OD1 | ASP | A | 347 | 24.214 | 68.084 | 4.718 | 1.00 | 42.71 |
| 92 | OD2 | ASP | A | 347 | 22.646 | 69.472 | 4.056 | 1.00 | 50.58 |
| 93 | C | ASP | A | 347 | 19.564 | 66.718 | 4.762 | 1.00 | 33.62 |
| 94 | O | ASP | A | 347 | 19.201 | 66.219 | 3.699 | 1.00 | 30.10 |
| 95 | N | LEU | A | 348 | 19.137 | 66.288 | 5.940 | 1.00 | 34.23 |
| 96 | CA | LEU | A | 348 | 18.226 | 65.163 | 6.054 | 1.00 | 38.75 |
| 97 | CE | LEU | A | 348 | 18.260 | 64.636 | 7.496 | 1.00 | 38.06 |
| 98 | CG | LEU | A | 348 | 17.363 | 63.435 | 7.811 | 1.00 | 43.84 |
| 99 | CD1 | LEU | A | 348 | 17.739 | 62.243 | 6.910 | 1.00 | 39.77 |
| 100 | CD2 | LEU | A | 348 | 17.510 | 63.064 | 9.291 | 1.00 | 43.35 |
| 101 | C | LEU | A | 348 | 16.763 | 65.439 | 5.645 | 1.00 | 39.57 |
| 102 | O | LEU | A | 348 | 16.146 | 64.621 | 4.959 | 1.00 | 38.51 |
| 103 | N | PHE | A | 349 | 16.214 | 66.583 | 6.057 | 1.00 | 40.41 |
| 104 | CA | PHE | A | 349 | 14.809 | 66.901 | 5.762 | 1.00 | 42.87 |
| 105 | CB | PHE | A | 349 | 14.111 | 67.364 | 7.040 | 1.00 | 39.59 |
| 106 | CG | PHE | A | 349 | 14.208 | 66.383 | 8.163 | 1.00 | 37.95 |
| 107 | CD1 | PHE | A | 349 | 15.004 | 66.649 | 9.268 | 1.00 | 36.40 |
| 108 | CD2 | PHE | A | 349 | 13.517 | 65.181 | 8.105 | 1.00 | 40.49 |
| 109 | CE1 | PHE | A | 349 | 15.116 | 65.726 | 10.305 | 1.00 | 39.63 |
| 110 | CE2 | PHE | A | 349 | 13.619 | 64.247 | 9.135 | 1.00 | 38.12 |
| 111 | CZ | PHE | A | 349 | 14.418 | 64.520 | 10.237 | 1.00 | 41.86 |
| 112 | C | PHE | A | 349 | 14.472 | 67.896 | 4.654 | 1.00 | 44.67 |
| 113 | O | PHE | A | 349 | 13.433 | 67.773 | 4.001 | 1.00 | 47.25 |
| 114 | N | ILE | A | 350 | 15.314 | 68.895 | 4.450 | 1.00 | 46.06 |
| 115 | CA | ILE | A | 350 | 15.027 | 69.871 | 3.417 | 1.00 | 50.35 |
| 116 | CB | ILE | A | 350 | 15.548 | 71.270 | 3.813 | 1.00 | 50.30 |
| 117 | CG2 | ILE | A | 350 | 14.997 | 72.316 | 2.864 | 1.00 | 51.91 |
| 118 | CG1 | ILE | A | 350 | 15.146 | 71.593 | 5.261 | 1.00 | 51.78 |
| 119 | CD1 | ILE | A | 350 | 13.685 | 71.366 | 5.580 | 1.00 | 45.73 |
| 120 | C | ILE | A | 350 | 15.694 | 69.421 | 2.127 | 1.00 | 51.95 |
| 121 | O | ILE | A | 350 | 15.028 | 69.146 | 1.130 | 1.00 | 52.71 |
| 122 | N | ARG | A | 351 | 17.016 | 69.319 | 2.173 | 1.00 | 53.39 |
| 123 | CA | ARG | A | 351 | 17.813 | 68.909 | 1.031 | 1.00 | 54.67 |
| 124 | CB | ARG | A | 351 | 19.290 | 69.059 | 1.393 | 1.00 | 59.14 |
| 125 | CO | ARG | A | 351 | 20.186 | 69.537 | 0.267 | 1.00 | 64.87 |
| 126 | CD | ARG | A | 351 | 21.254 | 70.473 | 0.808 | 1.00 | 66.51 |
| 127 | NE | ARG | A | 351 | 22.405 | 70.566 | −0.080 | 1.00 | 73.42 |
| 128 | CZ | ARG | A | 351 | 23.263 | 69.573 | −0.283 | 1.00 | 78.26 |
| 129 | NH1 | ARG | A | 351 | 23.094 | 68.417 | 0.344 | 1.00 | 81.30 |
| 130 | NH2 | ARG | A | 351 | 24.288 | 69.733 | −1.111 | 1.00 | 79.63 |
| 131 | C | ARG | A | 351 | 17.508 | 67.468 | 0.616 | 1.00 | 53.92 |
| 132 | O | ARG | A | 351 | 17.510 | 67.144 | −0.567 | 1.00 | 53.66 |
| 133 | N | LYS | A | 352 | 17.248 | 66.612 | 1.598 | 1.00 | 53.41 |
| 134 | CA | LYE | A | 352 | 16.942 | 65.200 | 1.360 | 1.00 | 53.02 |
| 135 | CE | LYS | A | 352 | 15.779 | 65.053 | 0.375 | 1.00 | 54.25 |
| 136 | CG | LYS | A | 352 | 14.506 | 65.752 | 0.814 | 1.00 | 64.61 |
| 137 | CD | LYS | A | 352 | 13.366 | 65.442 | −0.146 | 1.00 | 71.92 |
| 138 | CE | LYE | A | 352 | 12.202 | 66.410 | 0.014 | 1.00 | 76.75 |
| 139 | NZ | LYS | A | 352 | 12.573 | 67.793 | −0.405 | 1.00 | 82.42 |
| 140 | C | LYE | A | 352 | 18.130 | 64.383 | 0.847 | 1.00 | 51.14 |
| 141 | O | LYS | A | 352 | 17.945 | 63.417 | 0.109 | 1.00 | 51.55 |
| 142 | N | SER | A | 353 | 19.341 | 64.772 | 1.237 | 1.00 | 47.69 |
| 143 | CA | SER | A | 353 | 20.552 | 64.067 | 0.832 | 1.00 | 45.50 |
| 144 | CE | SER | A | 353 | 21.154 | 64.718 | −0.418 | 1.00 | 50.28 |
| 145 | OG | SER | A | 353 | 21.538 | 66.058 | −0.171 | 1.00 | 63.45 |
| 146 | C | SER | A | 353 | 21.528 | 64.135 | 2.006 | 1.00 | 40.39 |
| 147 | O | SER | A | 353 | 22.498 | 64.890 | 1.995 | 1.00 | 40.20 |
| 148 | N | PRO | A | 354 | 21.268 | 63.329 | 3.045 | 1.00 | 36.74 |
| 149 | CD | PRO | A | 354 | 20.188 | 62.338 | 3.039 | 1.00 | 36.52 |
| 150 | CA | PRO | A | 354 | 22.043 | 63.224 | 4.283 | 1.00 | 34.20 |
| 151 | CB | PRO | A | 354 | 21.182 | 62.317 | 5.163 | 1.00 | 31.70 |
| 152 | CO | PRO | A | 354 | 19.870 | 62.260 | 4.472 | 1.00 | 40.26 |
| 153 | C | PRO | A | 354 | 23.433 | 62.636 | 4.121 | 1.00 | 32.13 |
| 154 | O | PRO | A | 354 | 23.655 | 61.771 | 3.283 | 1.00 | 28.54 |
| 155 | N | THR | A | 355 | 24.359 | 63.111 | 4.942 | 1.00 | 30.96 |
| 156 | CA | THR | A | 355 | 25.718 | 62.601 | 4.936 | 1.00 | 31.86 |
| 157 | CB | THR | A | 355 | 26.692 | 63.501 | 4.121 | 1.00 | 32.67 |
| 158 | OG1 | THR | A | 355 | 26.806 | 64.777 | 4.752 | 1.00 | 34.86 |
| 159 | CG2 | THR | A | 355 | 26.194 | 63.706 | 2.699 | 1.00 | 34.90 |
| 160 | C | THR | A | 355 | 26.221 | 62.548 | 6.374 | 1.00 | 33.38 |
| 161 | O | THR | A | 355 | 25.703 | 63.239 | 7.260 | 1.00 | 32.61 |
| 162 | N | ILE | A | 356 | 27.197 | 61.691 | 6.618 | 1.00 | 32.83 |
| 163 | CA | ILE | A | 356 | 27.793 | 61.641 | 7.935 | 1.00 | 33.21 |
| 164 | CB | ILE | A | 356 | 27.538 | 60.303 | 8.666 | 1.00 | 34.88 |
| 165 | CG2 | ILE | A | 356 | 26.038 | 60.142 | 8.910 | 1.00 | 34.84 |
| 166 | CG1 | ILE | A | 356 | 28.098 | 59.135 | 7.860 | 1.00 | 37.26 |
| 167 | CD1 | ILE | A | 356 | 27.809 | 57.757 | 8.489 | 1.00 | 38.06 |
| 168 | C | ILE | A | 356 | 29.265 | 61.855 | 7.639 | 1.00 | 32.48 |
| 169 | O | ILE | A | 356 | 29.739 | 61.563 | 6.536 | 1.00 | 30.30 |
| 170 | N | THR | A | 357 | 29.991 | 62.364 | 8.622 | 1.00 | 33.59 |
| 171 | CA | THR | A | 357 | 31.389 | 62.671 | 8.406 | 1.00 | 32.83 |
| 172 | CB | THR | A | 357 | 31.575 | 64.216 | 8.243 | 1.00 | 37.46 |
| 173 | OG1 | THR | A | 357 | 30.985 | 64.645 | 7.009 | 1.00 | 35.11 |
| 174 | CG2 | THR | A | 357 | 33.047 | 64.599 | 8.258 | 1.00 | 40.13 |
| 175 | C | THR | A | 357 | 32.274 | 62.180 | 9.530 | 1.00 | 34.22 |
| 176 | O | THR | A | 357 | 31.991 | 62.378 | 10.716 | 1.00 | 33.99 |
| 177 | N | CYS | A | 358 | 33.369 | 61.555 | 9.134 | 1.00 | 34.04 |
| 178 | CA | CYS | A | 358 | 34.344 | 61.037 | 10.068 | 1.00 | 36.90 |
| 179 | C | CYS | A | 358 | 35.496 | 62.027 | 9.999 | 1.00 | 36.26 |
| 180 | O | CYS | A | 358 | 36.103 | 62.188 | 8.944 | 1.00 | 35.68 |
| 181 | CB | CYS | A | 358 | 34.809 | 59.670 | 9.593 | 1.00 | 39.83 |
| 182 | SG | CYS | A | 358 | 35.781 | 58.719 | 10.798 | 1.00 | 52.73 |
| 183 | N | LEU | A | 359 | 35.780 | 62.692 | 11.112 | 1.00 | 38.57 |
| 184 | CA | LEU | A | 359 | 36.849 | 63.679 | 11.178 | 1.00 | 42.35 |
| 185 | CB | LEU | A | 359 | 36.327 | 64.985 | 11.796 | 1.00 | 42.11 |
| 186 | CG | LEU | A | 359 | 37.373 | 66.028 | 12.219 | 1.00 | 48.42 |
| 187 | CD1 | LEU | A | 359 | 38.221 | 66.454 | 11.027 | 1.00 | 49.46 |
| 188 | CD2 | LEU | A | 359 | 36.666 | 67.230 | 12.831 | 1.00 | 48.09 |
| 189 | C | LEU | A | 359 | 38.011 | 63.163 | 12.005 | 1.00 | 45.39 |
| 190 | O | LEU | A | 359 | 37.834 | 62.806 | 13.165 | 1.00 | 48.73 |
| 191 | N | VAL | A | 360 | 39.195 | 63.121 | 11.407 | 1.00 | 47.72 |
| 192 | CA | VAL | A | 360 | 40.395 | 62.669 | 12.104 | 1.00 | 51.87 |
| 193 | CB | VAL | A | 360 | 41.086 | 61.525 | 11.335 | 1.00 | 52.99 |
| 194 | CG1 | VAL | A | 360 | 42.365 | 61.110 | 12.049 | 1.00 | 51.29 |
| 195 | CG2 | VAL | A | 360 | 40.144 | 60.348 | 11.210 | 1.00 | 52.42 |
| 196 | C | VAL | A | 360 | 41.379 | 63.833 | 12.226 | 1.00 | 55.21 |
| 197 | O | VAL | A | 360 | 41.634 | 64.537 | 11.247 | 1.00 | 56.01 |
| 198 | N | VAL | A | 361 | 41.920 | 64.042 | 13.423 | 1.00 | 57.47 |
| 199 | CA | VAL | A | 361 | 42.882 | 65.121 | 13.652 | 1.00 | 61.75 |
| 200 | CB | VAL | A | 361 | 42.352 | 66.153 | 14.675 | 1.00 | 58.91 |
| 201 | CG1 | VAL | A | 361 | 43.389 | 67.232 | 14.913 | 1.00 | 56.98 |
| 202 | CG2 | VAL | A | 361 | 41.070 | 66.774 | 14.166 | 1.00 | 54.49 |
| 203 | C | VAL | A | 361 | 44.197 | 64.555 | 14.177 | 1.00 | 66.78 |
| 204 | O | VAL | A | 361 | 44.208 | 63.764 | 15.119 | 1.00 | 67.30 |
| 205 | N | ASP | A | 362 | 45.301 | 64.959 | 13.556 | 1.00 | 72.35 |
| 206 | CA | ASP | A | 362 | 46.631 | 64.500 | 13.955 | 1.00 | 79.16 |
| 207 | CB | ASP | A | 362 | 47.334 | 63.825 | 12.769 | 1.00 | 83.66 |
| 208 | CG | ASP | A | 362 | 48.603 | 63.089 | 13.176 | 1.00 | 87.40 |
| 209 | OD1 | ASP | A | 362 | 49.071 | 63.283 | 14.320 | 1.00 | 91.40 |
| 210 | OD2 | ASP | A | 362 | 49.138 | 62.318 | 12.348 | 1.00 | 87.33 |
| 211 | C | ASP | A | 362 | 47.444 | 65.704 | 14.424 | 1.00 | 82.84 |
| 212 | O | ASP | A | 362 | 47.937 | 66.487 | 13.607 | 1.00 | 84.14 |
| 213 | N | ALA | A | 363 | 47.578 | 65.849 | 15.740 | 1.00 | 85.70 |
| 214 | CA | ALA | A | 363 | 48.319 | 66.964 | 16.325 | 1.00 | 89.05 |
| 215 | CB | ALA | A | 363 | 48.446 | 66.770 | 17.830 | 1.00 | 90.18 |
| 216 | C | ALA | A | 363 | 49.701 | 67.142 | 15.699 | 1.00 | 91.80 |
| 217 | O | ALA | A | 363 | 50.243 | 68.246 | 15.691 | 1.00 | 92.23 |
| 218 | N | ALA | A | 364 | 50.263 | 66.056 | 15.174 | 1.00 | 94.23 |
| 219 | CA | ALA | A | 364 | 51.578 | 66.085 | 14.536 | 1.00 | 97.42 |
| 220 | CB | ALA | A | 364 | 52.668 | 66.159 | 15.594 | 1.00 | 96.05 |
| 221 | C | ALA | A | 364 | 51.755 | 64.828 | 13.687 | 1.00 | 100.00 |
| 222 | O | ALA | A | 364 | 51.767 | 63.715 | 14.213 | 1.00 | 101.29 |
| 223 | N | PRO | A | 365 | 51.885 | 64.988 | 12.359 | 1.00 | 101.76 |
| 224 | CD | PRO | A | 365 | 51.407 | 66.158 | 11.597 | 1.00 | 101.62 |
| 225 | CA | PRO | A | 365 | 52.053 | 63.836 | 11.468 | 1.00 | 103.14 |
| 226 | CB | PRO | A | 365 | 51.022 | 64.114 | 10.394 | 1.00 | 103.27 |
| 227 | CG | PRO | A | 365 | 51.228 | 65.594 | 10.183 | 1.00 | 103.12 |
| 228 | C | PRO | A | 365 | 53.456 | 63.681 | 10.872 | 1.00 | 104.03 |
| 229 | O | PRO | A | 365 | 54.419 | 63.372 | 11.579 | 1.00 | 104.47 |
| 230 | N | ALA | A | 366 | 53.536 | 63.884 | 9.557 | 1.00 | 104.37 |
| 231 | CA | ALA | A | 366 | 54.776 | 63.793 | 8.793 | 1.00 | 104.87 |
| 232 | CB | ALA | A | 366 | 55.839 | 64.705 | 9.413 | 1.00 | 105.36 |
| 233 | C | ALA | A | 366 | 55.319 | 62.370 | 8.665 | 1.00 | 104.90 |
| 234 | O | ALA | A | 366 | 56.461 | 62.108 | 9.045 | 1.00 | 105.14 |
| 235 | N | LYS | A | 367 | 54.514 | 61.452 | 8.129 | 1.00 | 104.05 |
| 236 | CA | LYS | A | 367 | 54.982 | 60.077 | 7.972 | 1.00 | 103.07 |
| 237 | CB | LYS | A | 367 | 55.483 | 59.539 | 9.315 | 1.00 | 103.33 |
| 238 | CG | LYS | A | 367 | 54.411 | 59.406 | 10.383 | 1.00 | 102.12 |
| 239 | CD | LYS | A | 367 | 54.951 | 58.638 | 11.572 | 1.00 | 103.99 |
| 240 | CE | LYS | A | 367 | 53.876 | 58.371 | 12.604 | 1.00 | 103.83 |
| 241 | NZ | LYS | A | 367 | 54.417 | 57.544 | 13.714 | 1.00 | 105.88 |
| 242 | C | LYS | A | 367 | 54.024 | 59.046 | 7.373 | 1.00 | 102.12 |
| 243 | O | LYS | A | 367 | 53.888 | 57.947 | 7.915 | 1.00 | 103.05 |
| 244 | N | GLY | A | 368 | 53.365 | 59.372 | 6.265 | 1.00 | 99.91 |
| 245 | CA | GLY | A | 368 | 52.480 | 58.388 | 5.664 | 1.00 | 96.70 |
| 246 | C | GLY | A | 368 | 51.079 | 58.800 | 5.255 | 1.00 | 94.03 |
| 247 | O | GLY | A | 368 | 50.829 | 59.949 | 4.891 | 1.00 | 94.76 |
| 248 | N | ALA | A | 369 | 50.162 | 57.839 | 5.313 | 1.00 | 90.44 |
| 249 | CA | ALA | A | 369 | 48.772 | 58.066 | 4.935 | 1.00 | 86.18 |
| 250 | CB | ALA | A | 369 | 48.460 | 57.308 | 3.645 | 1.00 | 86.97 |
| 251 | C | ALA | A | 369 | 47.808 | 57.639 | 6.038 | 1.00 | 82.79 |
| 252 | O | ALA | A | 369 | 48.149 | 56.827 | 6.897 | 1.00 | 83.46 |
| 253 | N | VAL | A | 370 | 46.600 | 58.193 | 6.001 | 1.00 | 78.82 |
| 254 | CA | VAL | A | 370 | 45.567 | 57.881 | 6.985 | 1.00 | 73.44 |
| 255 | CB | VAL | A | 370 | 45.161 | 59.132 | 7.777 | 1.00 | 71.37 |
| 256 | CG1 | VAL | A | 370 | 44.145 | 58.763 | 8.840 | 1.00 | 68.72 |
| 257 | CG2 | VAL | A | 370 | 46.388 | 59.769 | 8.404 | 1.00 | 66.32 |
| 258 | C | VAL | A | 370 | 44.347 | 57.346 | 6.252 | 1.00 | 71.19 |
| 259 | O | VAL | A | 370 | 43.803 | 58.019 | 5.383 | 1.00 | 70.77 |
| 260 | N | ASN | A | 371 | 43.913 | 56.138 | 6.605 | 1.00 | 69.11 |
| 261 | CA | ASN | A | 371 | 42.769 | 55.531 | 5.936 | 1.00 | 67.35 |
| 262 | CB | ASN | A | 371 | 43.145 | 54.138 | 5.430 | 1.00 | 68.95 |
| 263 | CG | ASN | A | 371 | 44.330 | 54.168 | 4.498 | 1.00 | 73.37 |
| 264 | OD1 | ASN | A | 371 | 44.299 | 54.828 | 3.459 | 1.00 | 76.52 |
| 265 | ND2 | ASN | A | 371 | 45.387 | 53.454 | 4.863 | 1.00 | 76.06 |
| 266 | C | ASN | A | 371 | 41.491 | 55.441 | 6.766 | 1.00 | 65.35 |
| 267 | O | ASN | A | 371 | 41.516 | 55.146 | 7.964 | 1.00 | 63.63 |
| 268 | N | LEU | A | 372 | 40.373 | 55.708 | 6.100 | 1.00 | 63.51 |
| 269 | CA | LEU | A | 372 | 39.060 | 55.651 | 6.721 | 1.00 | 60.50 |
| 270 | CB | LEU | A | 372 | 38.386 | 57.027 | 6.705 | 1.00 | 60.09 |
| 271 | CG | LEU | A | 372 | 39.139 | 58.170 | 7.386 | 1.00 | 61.37 |
| 272 | CD1 | LEU | A | 372 | 38.265 | 59.404 | 7.412 | 1.00 | 63.12 |
| 273 | CD2 | LEU | A | 372 | 39.517 | 57.771 | 8.791 | 1.00 | 62.53 |
| 274 | C | LEU | A | 372 | 38.220 | 54.658 | 5.936 | 1.00 | 57.54 |
| 275 | O | LEU | A | 372 | 38.069 | 54.776 | 4.720 | 1.00 | 58.06 |
| 276 | N | THR | A | 373 | 37.681 | 53.674 | 6.642 | 1.00 | 54.64 |
| 277 | CA | THR | A | 373 | 36.855 | 52.648 | 6.026 | 1.00 | 52.59 |
| 278 | CB | THR | A | 373 | 37.425 | 51.254 | 6.314 | 1.00 | 54.37 |
| 279 | OG1 | THR | A | 373 | 38.799 | 51.217 | 5.909 | 1.00 | 60.15 |
| 280 | CG2 | THR | A | 373 | 36.645 | 50.191 | 5.555 | 1.00 | 57.66 |
| 281 | C | THR | A | 373 | 35.444 | 52.722 | 6.591 | 1.00 | 48.12 |
| 282 | O | THR | A | 373 | 35.263 | 52.797 | 7.803 | 1.00 | 46.78 |
| 283 | N | TRP | A | 374 | 34.450 | 52.705 | 5.709 | 1.00 | 45.42 |
| 284 | CA | TRP | A | 374 | 33.057 | 52.767 | 6.129 | 1.00 | 44.33 |
| 285 | CS | TRP | A | 374 | 32.260 | 53.762 | 5.277 | 1.00 | 40.42 |
| 286 | CG | TRP | A | 374 | 32.664 | 55.183 | 5.442 | 1.00 | 41.22 |
| 287 | CD2 | TRP | A | 374 | 32.215 | 56.090 | 6.457 | 1.00 | 35.68 |
| 288 | CE2 | TRP | A | 374 | 32.841 | 57.329 | 6.219 | 1.00 | 31.67 |
| 289 | CE3 | TRP | A | 374 | 31.342 | 55.972 | 7.545 | 1.00 | 35.08 |
| 290 | CD1 | TRP | A | 374 | 33.523 | 55.888 | 4.652 | 1.00 | 38.77 |
| 291 | NE1 | TRP | A | 374 | 33.634 | 57.180 | 5.112 | 1.00 | 37.27 |
| 292 | CZ2 | TRP | A | 374 | 32.621 | 58.447 | 7.027 | 1.00 | 32.53 |
| 293 | CZ3 | TRP | A | 374 | 31.121 | 57.084 | 8.352 | 1.00 | 36.03 |
| 294 | CH2 | TRP | A | 374 | 31.760 | 58.304 | 8.087 | 1.00 | 33.34 |
| 295 | C | TRP | A | 374 | 32.385 | 51.405 | 6.022 | 1.00 | 44.84 |
| 296 | O | TRP | A | 374 | 32.758 | 50.581 | 5.186 | 1.00 | 43.54 |
| 297 | N | SER | A | 375 | 31.395 | 51.181 | 6.882 | 1.00 | 44.41 |
| 298 | CA | SER | A | 375 | 30.633 | 49.940 | 6.887 | 1.00 | 45.63 |
| 299 | CB | SER | A | 375 | 31.432 | 48.800 | 7.535 | 1.00 | 47.59 |
| 300 | OG | SER | A | 375 | 31.512 | 48.938 | 8.943 | 1.00 | 49.42 |
| 301 | C | SER | A | 375 | 29.308 | 50.112 | 7.618 | 1.00 | 45.23 |
| 302 | O | SER | A | 375 | 29.162 | 50.991 | 8.471 | 1.00 | 46.27 |
| 303 | N | ARG | A | 376 | 28.341 | 49.277 | 7.257 | 1.00 | 42.63 |
| 304 | CA | ARG | A | 376 | 27.033 | 49.307 | 7.882 | 1.00 | 41.96 |
| 305 | CB | ARG | A | 376 | 25.924 | 49.263 | 6.834 | 1.00 | 43.72 |
| 306 | CG | ARG | A | 376 | 25.855 | 50.458 | 5.901 | 1.00 | 40.62 |
| 307 | CD | ARG | A | 376 | 24.405 | 50.693 | 5.520 | 1.00 | 41.72 |
| 308 | NE | ARG | A | 376 | 24.143 | 50.468 | 4.110 | 1.00 | 52.53 |
| 309 | CZ | ARG | A | 376 | 22.929 | 50.305 | 3.599 | 1.00 | 52.81 |
| 310 | NH1 | ARG | A | 376 | 21.859 | 50.335 | 4.386 | 1.00 | 51.46 |
| 311 | NH2 | ARG | A | 376 | 22.781 | 50.128 | 2.293 | 1.00 | 56.61 |
| 312 | C | ARG | A | 376 | 26.920 | 48.082 | 8.775 | 1.00 | 39.96 |
| 313 | O | ARG | A | 376 | 27.218 | 46.971 | 8.355 | 1.00 | 40.84 |
| 314 | N | ALA | A | 377 | 26.503 | 48.285 | 10.014 | 1.00 | 41.64 |
| 315 | CA | ALA | A | 377 | 26.350 | 47.177 | 10.946 | 1.00 | 41.34 |
| 316 | CB | ALA | A | 377 | 25.705 | 47.680 | 12.227 | 1.00 | 33.50 |
| 317 | C | ALA | A | 377 | 25.502 | 46.056 | 10.325 | 1.00 | 41.22 |
| 318 | O | ALA | A | 377 | 25.685 | 44.888 | 10.648 | 1.00 | 41.04 |
| 319 | N | SER | A | 378 | 24.592 | 46.421 | 9.423 | 1.00 | 40.92 |
| 320 | CA | SER | A | 378 | 23.719 | 45.451 | 8.773 | 1.00 | 42.71 |
| 321 | CB | SER | A | 378 | 22.491 | 46.139 | 8.185 | 1.00 | 44.29 |
| 322 | OG | SER | A | 378 | 22.842 | 46.867 | 7.020 | 1.00 | 42.78 |
| 323 | C | SER | A | 378 | 24.420 | 44.706 | 7.658 | 1.00 | 43.58 |
| 324 | O | SER | A | 378 | 23.851 | 43.791 | 7.078 | 1.00 | 44.22 |
| 325 | N | GLY | A | 379 | 25.646 | 45.117 | 7.349 | 1.00 | 43.82 |
| 326 | CA | GLY | A | 379 | 26.409 | 44.463 | 6.304 | 1.00 | 45.23 |
| 327 | C | GLY | A | 379 | 26.060 | 44.917 | 4.901 | 1.00 | 48.43 |
| 328 | O | GLY | A | 379 | 26.747 | 44.562 | 3.943 | 1.00 | 47.67 |
| 329 | N | LYS | A | 380 | 24.995 | 45.699 | 4.763 | 1.00 | 50.04 |
| 330 | CA | LYS | A | 380 | 24.603 | 46.177 | 3.447 | 1.00 | 52.22 |
| 331 | CB | LYS | A | 380 | 23.238 | 46.868 | 3.509 | 1.00 | 54.82 |
| 332 | CG | LYS | A | 380 | 22.096 | 45.879 | 3.646 | 1.00 | 63.15 |
| 333 | CD | LYS | A | 380 | 20.734 | 46.528 | 3.466 | 1.00 | 70.67 |
| 334 | CE | LYS | A | 380 | 19.640 | 45.467 | 3.455 | 1.00 | 75.50 |
| 335 | NZ | LYS | A | 380 | 18.275 | 46.051 | 3.354 | 1.00 | 80.58 |
| 336 | C | LYS | A | 380 | 25.655 | 47.107 | 2.851 | 1.00 | 52.26 |
| 337 | O | LYS | A | 380 | 26.495 | 47.656 | 3.565 | 1.00 | 49.96 |
| 338 | N | PRO | A | 381 | 25.626 | 47.285 | 1.522 | 1.00 | 53.55 |
| 339 | CD | PRO | A | 381 | 24.694 | 46.657 | 0.571 | 1.00 | 54.43 |
| 340 | CA | PRO | A | 381 | 26.584 | 48.147 | 0.818 | 1.00 | 54.97 |
| 341 | CB | PRO | A | 381 | 26.212 | 47.948 | −0.655 | 1.00 | 57.06 |
| 342 | CG | PRO | A | 381 | 25.519 | 46.611 | −0.673 | 1.00 | 59.10 |
| 343 | C | PRO | A | 381 | 26.532 | 49.626 | 1.207 | 1.00 | 54.14 |
| 344 | O | PRO | A | 381 | 25.457 | 50.178 | 1.467 | 1.00 | 55.04 |
| 345 | N | VAL | A | 382 | 27.701 | 50.256 | 1.251 | 1.00 | 52.78 |
| 346 | CA | VAL | A | 382 | 27.801 | 51.680 | 1.546 | 1.00 | 53.97 |
| 347 | CB | VAL | A | 382 | 28.891 | 51.997 | 2.611 | 1.00 | 54.50 |
| 348 | CG1 | VAL | A | 382 | 28.595 | 51.252 | 3.896 | 1.00 | 56.74 |
| 349 | CG2 | VAL | A | 382 | 30.281 | 51.645 | 2.078 | 1.00 | 46.72 |
| 350 | C | VAL | A | 382 | 28.202 | 52.338 | 0.226 | 1.00 | 54.10 |
| 351 | O | VAL | A | 382 | 28.910 | 51.735 | −0.583 | 1.00 | 53.38 |
| 352 | N | ASN | A | 383 | 27.752 | 53.566 | 0.011 | 1.00 | 53.55 |
| 353 | CA | ASN | A | 383 | 28.073 | 54.289 | −1.210 | 1.00 | 54.35 |
| 354 | CB | ASN | A | 383 | 27.135 | 55.485 | −1.370 | 1.00 | 60.43 |
| 355 | CG | ASN | A | 383 | 25.677 | 55.087 | −1.371 | 1.00 | 67.43 |
| 356 | OD1 | ASN | A | 383 | 24.806 | 55.890 | −1.037 | 1.00 | 75.26 |
| 357 | ND2 | ASN | A | 383 | 25.399 | 53.845 | −1.756 | 1.00 | 72.99 |
| 358 | C | ASN | A | 383 | 29.520 | 54.779 | −1.218 | 1.00 | 52.64 |
| 359 | O | ASN | A | 383 | 30.292 | 54.518 | −0.293 | 1.00 | 50.26 |
| 360 | N | HIS | A | 384 | 29.879 | 55.493 | −2.276 | 1.00 | 51.74 |
| 361 | CA | HIS | A | 384 | 31.225 | 56.038 | −2.415 | 1.00 | 51.28 |
| 362 | CB | HIS | A | 384 | 31.499 | 56.380 | −3.879 | 1.00 | 59.06 |
| 363 | CG | HIS | A | 384 | 31.657 | 55.179 | −4.761 | 1.00 | 65.42 |
| 364 | CD2 | HIS | A | 384 | 30.912 | 54.727 | −5.796 | 1.00 | 72.26 |
| 365 | ND1 | HIS | A | 384 | 32.694 | 54.284 | −4.615 | 1.00 | 70.87 |
| 366 | CE1 | HIS | A | 384 | 32.581 | 53.330 | −5.523 | 1.00 | 76.58 |
| 367 | NE2 | HIS | A | 384 | 31.507 | 53.576 | −6.253 | 1.00 | 77.95 |
| 368 | C | HIS | A | 384 | 31.362 | 57.294 | −1.555 | 1.00 | 47.99 |
| 369 | O | HIS | A | 384 | 30.478 | 58.145 | −1.544 | 1.00 | 45.37 |
| 370 | N | SER | A | 385 | 32.473 | 57.413 | −0.844 | 1.00 | 45.57 |
| 371 | CA | SER | A | 385 | 32.674 | 58.564 | 0.015 | 1.00 | 45.69 |
| 372 | CB | SER | A | 385 | 33.134 | 58.097 | 1.405 | 1.00 | 41.19 |
| 373 | OG | SER | A | 385 | 34.299 | 57.285 | 1.338 | 1.00 | 41.34 |
| 374 | C | SER | A | 385 | 33.654 | 59.598 | −0.560 | 1.00 | 44.94 |
| 375 | O | SER | A | 385 | 34.413 | 59.313 | −1.482 | 1.00 | 41.64 |
| 376 | N | THR | A | 386 | 33.603 | 60.805 | −0.007 | 1.00 | 45.73 |
| 377 | CA | THR | A | 386 | 34.467 | 61.898 | −0.421 | 1.00 | 46.08 |
| 378 | CB | THR | A | 386 | 33.670 | 63.191 | −0.637 | 1.00 | 47.42 |
| 379 | OG1 | THR | A | 386 | 32.775 | 63.024 | −1.744 | 1.00 | 50.45 |
| 380 | CC2 | THR | A | 386 | 34.615 | 64.347 | −0.925 | 1.00 | 51.73 |
| 381 | C | THR | A | 386 | 35.487 | 62.132 | 0.681 | 1.00 | 46.45 |
| 382 | O | THR | A | 386 | 35.129 | 62.241 | 1.853 | 1.00 | 47.06 |
| 383 | N | ARG | A | 387 | 36.754 | 62.206 | 0.292 | 1.00 | 46.33 |
| 384 | CA | ARG | A | 387 | 37.849 | 62.410 | 1.228 | 1.00 | 46.50 |
| 385 | CB | ARG | A | 387 | 38.874 | 61.290 | 1.030 | 1.00 | 48.08 |
| 386 | CC | ARG | A | 387 | 40.086 | 61.366 | 1.933 | 1.00 | 54.89 |
| 387 | CD | AEG | A | 387 | 41.208 | 60.481 | 1.405 | 1.00 | 59.67 |
| 388 | NE | ARG | A | 387 | 42.438 | 60.668 | 2.165 | 1.00 | 63.12 |
| 389 | CZ | ARG | A | 387 | 42.775 | 59.950 | 3.230 | 1.00 | 65.65 |
| 390 | NH1 | ARG | A | 387 | 41.971 | 58.982 | 3.651 | 1.00 | 67.29 |
| 391 | NH2 | ARG | A | 387 | 43.899 | 60.215 | 3.889 | 1.00 | 63.40 |
| 392 | C | ARG | A | 387 | 38.517 | 63.786 | 1.046 | 1.00 | 46.30 |
| 393 | O | ARG | A | 387 | 38.733 | 64.239 | −0.079 | 1.00 | 44.30 |
| 394 | N | LYS | A | 388 | 38.834 | 64.441 | 2.162 | 1.00 | 45.70 |
| 395 | CA | LYS | A | 388 | 39.489 | 65.749 | 2.140 | 1.00 | 44.68 |
| 396 | CB | LYS | A | 388 | 38.479 | 66.852 | 2.466 | 1.00 | 46.84 |
| 397 | CG | LYS | A | 388 | 37.337 | 66.931 | 1.476 | 1.00 | 56.94 |
| 398 | CD | LYS | A | 388 | 36.150 | 67.679 | 2.054 | 1.00 | 58.62 |
| 399 | CE | LYS | A | 388 | 34.883 | 67.307 | 1.309 | 1.00 | 61.92 |
| 400 | NZ | LYS | A | 388 | 33.676 | 67.856 | 1.977 | 1.00 | 70.07 |
| 401 | C | LYS | A | 388 | 40.646 | 65.798 | 3.143 | 1.00 | 44.52 |
| 402 | O | LYS | A | 388 | 40.502 | 65.379 | 4.294 | 1.00 | 40.60 |
| 403 | N | GLU | A | 389 | 41.793 | 66.304 | 2.689 | 1.00 | 47.26 |
| 404 | CA | GLU | A | 389 | 43.001 | 66.424 | 3.518 | 1.00 | 50.72 |
| 405 | CB | GLU | A | 389 | 44.114 | 65.531 | 2.965 | 1.00 | 54.88 |
| 406 | CG | GLU | A | 389 | 43.848 | 64.041 | 3.039 | 1.00 | 67.37 |
| 407 | CD | GLU | A | 389 | 44.979 | 63.225 | 2.436 | 1.00 | 75.83 |
| 408 | OE1 | GLU | A | 389 | 46.116 | 63.742 | 2.364 | 1.00 | 79.33 |
| 409 | OE2 | GLU | A | 389 | 44.738 | 62.062 | 2.044 | 1.00 | 80.70 |
| 410 | C | GLU | A | 389 | 43.510 | 67.871 | 3.551 | 1.00 | 51.11 |
| 411 | O | GLU | A | 389 | 43.644 | 68.510 | 2.504 | 1.00 | 48.28 |
| 412 | N | ALA | A | 390 | 43.814 | 68.382 | 4.741 | 1.00 | 52.07 |
| 413 | CA | ALA | A | 390 | 44.297 | 69.755 | 4.868 | 1.00 | 56.84 |
| 414 | CB | ALA | A | 390 | 43.120 | 70.712 | 4.972 | 1.00 | 53.86 |
| 415 | C | ALA | A | 390 | 45.229 | 69.957 | 6.058 | 1.00 | 60.96 |
| 416 | O | ALA | A | 390 | 45.085 | 69.315 | 7.098 | 1.00 | 61.14 |
| 417 | N | ALA | A | 391 | 46.179 | 70.873 | 5.896 | 1.00 | 65.61 |
| 418 | CA | ALA | A | 391 | 47.151 | 71.187 | 6.938 | 1.00 | 68.46 |
| 419 | CB | ALA | A | 391 | 48.398 | 70.336 | 6.761 | 1.00 | 71.73 |
| 420 | C | ALA | A | 391 | 47.518 | 72.661 | 6.866 | 1.00 | 70.37 |
| 421 | O | ALA | A | 391 | 47.076 | 73.461 | 7.688 | 1.00 | 72.17 |
| 422 | N | LEU | A | 397 | 47.315 | 68.210 | 11.080 | 1.00 | 60.70 |
| 423 | CA | LEU | A | 397 | 46.686 | 67.651 | 9.890 | 1.00 | 61.36 |
| 424 | CD | LEU | A | 397 | 47.489 | 66.458 | 9.375 | 1.00 | 64.28 |
| 425 | CG | LEU | A | 397 | 46.854 | 65.736 | 8.180 | 1.00 | 65.19 |
| 426 | CD1 | LEU | A | 397 | 46.802 | 66.670 | 6.981 | 1.00 | 65.40 |
| 427 | CD2 | LEU | A | 397 | 47.652 | 64.485 | 7.844 | 1.00 | 68.57 |
| 428 | C | LEU | A | 397 | 45.241 | 67.211 | 10.119 | 1.00 | 61.24 |
| 429 | O | LEU | A | 397 | 44.934 | 66.485 | 11.064 | 1.00 | 62.11 |
| 430 | N | THR | A | 398 | 44.360 | 67.654 | 9.233 | 1.00 | 59.83 |
| 431 | CA | THR | A | 398 | 42.950 | 67.309 | 9.305 | 1.00 | 58.04 |
| 432 | CD | THR | A | 398 | 42.061 | 68.572 | 9.263 | 1.00 | 58.72 |
| 433 | OG1 | THR | A | 398 | 42.171 | 69.278 | 10.506 | 1.00 | 64.28 |
| 434 | CG2 | THR | A | 398 | 40.608 | 68.197 | 9.023 | 1.00 | 61.55 |
| 435 | C | THR | A | 398 | 42.573 | 66.401 | 8.135 | 1.00 | 55.72 |
| 436 | O | THR | A | 398 | 43.021 | 66.595 | 7.000 | 1.00 | 55.02 |
| 437 | N | VAL | A | 399 | 41.752 | 65.401 | 8.422 | 1.00 | 52.38 |
| 438 | CA | VAL | A | 399 | 41.297 | 64.473 | 7.402 | 1.00 | 49.13 |
| 439 | CB | VAL | A | 399 | 42.143 | 63.179 | 7.391 | 1.00 | 51.57 |
| 440 | CG1 | VAL | A | 399 | 41.594 | 62.212 | 6.363 | 1.00 | 52.00 |
| 441 | CG2 | VAL | A | 399 | 43.590 | 63.500 | 7.068 | 1.00 | 55.81 |
| 442 | C | VAL | A | 399 | 39.847 | 64.093 | 7.643 | 1.00 | 46.27 |
| 443 | O | VAL | A | 399 | 39.478 | 63.649 | 8.732 | 1.00 | 43.17 |
| 444 | N | THR | A | 400 | 39.015 | 64.289 | 6.630 | 1.00 | 44.56 |
| 445 | CA | THR | A | 400 | 37.619 | 63.915 | 6.758 | 1.00 | 43.39 |
| 446 | CB | THR | A | 400 | 36.669 | 65.140 | 6.829 | 1.00 | 41.43 |
| 447 | OG1 | THR | A | 400 | 36.628 | 65.794 | 5.557 | 1.00 | 48.65 |
| 448 | CG2 | THR | A | 400 | 37.126 | 66.120 | 7.889 | 1.00 | 38.93 |
| 449 | C | THR | A | 400 | 37.174 | 63.059 | 5.584 | 1.00 | 40.85 |
| 450 | O | THR | A | 400 | 37.751 | 63.094 | 4.494 | 1.00 | 35.85 |
| 451 | N | SER | A | 401 | 36.146 | 62.267 | 5.847 | 1.00 | 39.13 |
| 452 | CA | SER | A | 401 | 35.542 | 61.417 | 4.847 | 1.00 | 37.93 |
| 453 | CB | SER | A | 401 | 35.885 | 59.944 | 5.079 | 1.00 | 40.15 |
| 454 | OG | SER | A | 401 | 35.290 | 59.142 | 4.069 | 1.00 | 44.51 |
| 455 | C | SER | A | 401 | 34.054 | 61.622 | 5.044 | 1.00 | 36.16 |
| 456 | O | SER | A | 401 | 33.552 | 61.461 | 6.153 | 1.00 | 36.19 |
| 457 | N | THR | A | 402 | 33.362 | 61.989 | 3.975 | 1.00 | 32.94 |
| 458 | CA | THR | A | 402 | 31.931 | 62.204 | 4.039 | 1.00 | 32.35 |
| 459 | CB | THR | A | 402 | 31.578 | 63.578 | 3.483 | 1.00 | 33.13 |
| 460 | OG1 | THR | A | 402 | 32.279 | 64.559 | 4.246 | 1.00 | 34.46 |
| 461 | CG2 | THR | A | 402 | 30.086 | 63.845 | 3.577 | 1.00 | 33.71 |
| 462 | C | THR | A | 402 | 31.222 | 61.117 | 3.243 | 1.00 | 31.66 |
| 463 | O | THR | A | 402 | 31.512 | 60.886 | 2.064 | 1.00 | 26.99 |
| 464 | N | LEU | A | 403 | 30.282 | 60.459 | 3.913 | 1.00 | 32.16 |
| 465 | CA | LEU | A | 403 | 29.526 | 59.370 | 3.319 | 1.00 | 32.46 |
| 466 | CB | LEU | A | 403 | 29.648 | 58.123 | 4.205 | 1.00 | 34.12 |
| 467 | CG | LEU | A | 403 | 28.920 | 56.838 | 3.794 | 1.00 | 34.78 |
| 468 | CD1 | LEU | A | 403 | 29.678 | 56.196 | 2.644 | 1.00 | 35.87 |
| 469 | CD2 | LEU | A | 403 | 28.856 | 55.873 | 4.968 | 1.00 | 31.42 |
| 470 | C | LEU | A | 403 | 28.052 | 59.690 | 3.099 | 1.00 | 33.39 |
| 471 | O | LEU | A | 403 | 27.321 | 60.005 | 4.040 | 1.00 | 30.14 |
| 472 | N | PRO | A | 404 | 27.604 | 59.633 | 1.835 | 1.00 | 34.13 |
| 473 | CD | PRO | A | 404 | 28.391 | 59.413 | 0.605 | 1.00 | 34.29 |
| 474 | CA | PRO | A | 404 | 26.203 | 59.899 | 1.513 | 1.00 | 34.91 |
| 475 | CB | PRO | A | 404 | 26.161 | 59.777 | −0.008 | 1.00 | 33.92 |
| 476 | CG | PRO | A | 404 | 27.554 | 60.121 | −0.426 | 1.00 | 33.77 |
| 477 | C | PRO | A | 404 | 25.427 | 58.773 | 2.191 | 1.00 | 36.43 |
| 478 | O | PRO | A | 404 | 25.824 | 57.610 | 2.140 | 1.00 | 30.87 |
| 479 | N | VAL | A | 405 | 24.326 | 59.120 | 2.835 | 1.00 | 37.52 |
| 480 | CA | VAL | A | 405 | 23.532 | 58.129 | 3.531 | 1.00 | 41.37 |
| 481 | CB | VAL | A | 405 | 23.522 | 58.462 | 5.053 | 1.00 | 44.39 |
| 482 | CG1 | VAL | A | 405 | 22.100 | 58.566 | 5.587 | 1.00 | 47.81 |
| 483 | CG2 | VAL | A | 405 | 24.312 | 57.420 | 5.799 | 1.00 | 43.63 |
| 484 | C | VAL | A | 405 | 22.108 | 58.056 | 2.980 | 1.00 | 42.93 |
| 485 | O | VAL | A | 405 | 21.558 | 59.047 | 2.511 | 1.00 | 42.10 |
| 486 | N | GLY | A | 406 | 21.518 | 56.868 | 3.019 | 1.00 | 45.32 |
| 487 | CA | GLY | A | 406 | 20.154 | 56.731 | 2.547 | 1.00 | 46.40 |
| 488 | C | GLY | A | 406 | 19.227 | 57.392 | 3.552 | 1.00 | 46.70 |
| 489 | O | GLY | A | 406 | 19.421 | 57.255 | 4.766 | 1.00 | 41.89 |
| 490 | N | THR | A | 407 | 18.227 | 58.114 | 3.048 | 1.00 | 46.37 |
| 491 | CA | THR | A | 407 | 17.259 | 58.809 | 3.892 | 1.00 | 49.05 |
| 492 | CB | THR | A | 407 | 16.252 | 59.617 | 3.038 | 1.00 | 51.01 |
| 493 | OG1 | THR | A | 407 | 16.929 | 60.700 | 2.391 | 1.00 | 55.76 |
| 494 | CG2 | THR | A | 407 | 15.147 | 60.177 | 3.905 | 1.00 | 54.66 |
| 495 | C | THR | A | 407 | 16.468 | 57.854 | 4.775 | 1.00 | 49.24 |
| 496 | O | THR | A | 407 | 16.446 | 58.003 | 5.995 | 1.00 | 48.91 |
| 497 | N | ALA | A | 408 | 15.823 | 56.874 | 4.147 | 1.00 | 49.71 |
| 498 | CA | ALA | A | 408 | 15.008 | 55.907 | 4.860 | 1.00 | 50.13 |
| 499 | CB | ALA | A | 408 | 14.233 | 55.038 | 3.876 | 1.00 | 53.74 |
| 500 | C | ALA | A | 408 | 15.823 | 55.031 | 5.788 | 1.00 | 51.25 |
| 501 | O | ALA | A | 408 | 15.393 | 54.748 | 6.909 | 1.00 | 50.28 |
| 502 | N | ASP | A | 409 | 16.995 | 54.600 | 5.330 | 1.00 | 50.74 |
| 503 | CA | ASP | A | 409 | 17.852 | 53.748 | 6.152 | 1.00 | 52.13 |
| 504 | CB | ASP | A | 409 | 19.102 | 53.335 | 5.370 | 1.00 | 58.27 |
| 505 | CG | ASP | A | 409 | 18.770 | 52.535 | 4.123 | 1.00 | 69.38 |
| 506 | OD1 | ASP | A | 409 | 18.105 | 51.479 | 4.252 | 1.00 | 75.94 |
| 507 | OD2 | ASP | A | 409 | 19.169 | 52.959 | 3.016 | 1.00 | 72.00 |
| 508 | C | ASP | A | 409 | 18.259 | 54.449 | 7.445 | 1.00 | 49.96 |
| 509 | O | ASP | A | 409 | 18.317 | 53.831 | 8.512 | 1.00 | 49.48 |
| 510 | N | TRP | A | 410 | 18.542 | 55.742 | 7.345 | 1.00 | 46.74 |
| 511 | CA | TRP | A | 410 | 18.931 | 56.494 | 8.517 | 1.00 | 44.75 |
| 512 | CB | TRP | A | 410 | 19.487 | 57.868 | 8.143 | 1.00 | 39.75 |
| 513 | CG | TRP | A | 410 | 19.913 | 58.601 | 9.362 | 1.00 | 37.35 |
| 514 | CD2 | TRP | A | 410 | 21.131 | 58.409 | 10.083 | 1.00 | 31.03 |
| 515 | CE2 | TRP | A | 410 | 21.049 | 59.188 | 11.254 | 1.00 | 29.68 |
| 516 | CE3 | TRP | A | 410 | 22.286 | 57.637 | 9.857 | 1.00 | 32.46 |
| 517 | CD1 | TRP | A | 410 | 19.166 | 59.481 | 10.099 | 1.00 | 32.61 |
| 518 | NE1 | TRP | A | 410 | 19.842 | 59.833 | 11.232 | 1.00 | 33.45 |
| 519 | CZ2 | TRP | A | 410 | 22.078 | 59.230 | 12.200 | 1.00 | 29.11 |
| 520 | CZ3 | TRP | A | 410 | 23.310 | 57.680 | 10.798 | 1.00 | 34.12 |
| 521 | CH2 | TRP | A | 410 | 23.195 | 58.472 | 11.958 | 1.00 | 35.80 |
| 522 | C | TRP | A | 410 | 17.743 | 56.666 | 9.451 | 1.00 | 43.08 |
| 523 | O | TRP | A | 410 | 17.867 | 56.463 | 10.655 | 1.00 | 38.95 |
| 524 | N | ILE | A | 411 | 16.593 | 57.037 | 8.894 | 1.00 | 46.39 |
| 525 | CA | ILE | A | 411 | 15.389 | 57.228 | 9.696 | 1.00 | 51.22 |
| 526 | CB | ILE | A | 411 | 14.218 | 57.752 | 8.841 | 1.00 | 53.02 |
| 527 | CG2 | ILE | A | 411 | 12.972 | 57.895 | 9.699 | 1.00 | 55.27 |
| 528 | CG1 | ILE | A | 411 | 14.590 | 59.112 | 8.240 | 1.00 | 56.63 |
| 529 | CD1 | ILE | A | 411 | 13.498 | 59.742 | 7.367 | 1.00 | 61.16 |
| 530 | C | ILE | A | 411 | 14.968 | 55.931 | 10.371 | 1.00 | 52.33 |
| 531 | O | ILE | A | 411 | 14.404 | 55.955 | 11.462 | 1.00 | 53.32 |
| 532 | N | GLU | A | 412 | 15.267 | 54.800 | 9.731 | 1.00 | 54.18 |
| 533 | CA | GLU | A | 412 | 14.907 | 53.501 | 10.279 | 1.00 | 53.56 |
| 534 | CB | GLU | A | 412 | 14.692 | 52.490 | 9.158 | 1.00 | 58.95 |
| 535 | CG | GUI | A | 412 | 13.298 | 52.566 | 8.572 | 1.00 | 73.61 |
| 536 | CD | GLU | A | 412 | 13.058 | 51.538 | 7.488 | 1.00 | 83.01 |
| 537 | OE1 | GLU | A | 412 | 13.415 | 50.356 | 7.689 | 1.00 | 89.18 |
| 538 | OE2 | GLU | A | 412 | 12.498 | 51.909 | 6.435 | 1.00 | 89.24 |
| 539 | C | GLU | A | 412 | 15.874 | 52.935 | 11.307 | 1.00 | 51.42 |
| 540 | O | GLU | A | 412 | 15.615 | 51.875 | 11.873 | 1.00 | 50.68 |
| 541 | N | GLY | A | 413 | 16.983 | 53.633 | 11.549 | 1.00 | 48.27 |
| 542 | CA | GLY | A | 413 | 17.929 | 53.172 | 12.549 | 1.00 | 46.15 |
| 543 | C | GLY | A | 413 | 19.223 | 52.489 | 12.133 | 1.00 | 44.58 |
| 544 | O | GLY | A | 413 | 19.925 | 51.963 | 12.994 | 1.00 | 42.91 |
| 545 | N | GLU | A | 414 | 19.556 | 52.482 | 10.844 | 1.00 | 44.06 |
| 546 | CA | GLU | A | 414 | 20.801 | 51.851 | 10.415 | 1.00 | 42.21 |
| 547 | CB | GLU | A | 414 | 21.077 | 52.133 | 8.928 | 1.00 | 42.27 |
| 548 | CG | GLU | A | 414 | 22.441 | 51.637 | 8.402 | 1.00 | 44.30 |
| 549 | CD | GLU | A | 414 | 22.564 | 50.113 | 8.361 | 1.00 | 52.18 |
| 550 | OE1 | GLU | A | 414 | 23.038 | 49.517 | 9.355 | 1.00 | 51.39 |
| 551 | OE2 | GLU | A | 414 | 22.175 | 49.510 | 7.336 | 1.00 | 49.53 |
| 552 | C | GLU | A | 414 | 21.910 | 52.453 | 11.261 | 1.00 | 41.40 |
| 553 | O | GLU | A | 414 | 21.819 | 53.605 | 11.687 | 1.00 | 38.41 |
| 554 | N | THR | A | 415 | 22.944 | 51.664 | 11.527 | 1.00 | 41.90 |
| 555 | CA | THR | A | 415 | 24.078 | 52.146 | 12.294 | 1.00 | 43.26 |
| 556 | CB | THR | A | 415 | 24.259 | 51.319 | 13.594 | 1.00 | 46.09 |
| 557 | OG1 | THR | A | 415 | 25.604 | 51.445 | 14.068 | 1.00 | 50.78 |
| 558 | CG2 | THR | A | 415 | 23.910 | 49.884 | 13.366 | 1.00 | 56.55 |
| 559 | C | THR | A | 415 | 25.330 | 52.101 | 11.410 | 1.00 | 41.87 |
| 560 | O | THR | A | 415 | 25.637 | 51.067 | 10.815 | 1.00 | 43.61 |
| 561 | N | TYR | A | 416 | 26.029 | 53.229 | 11.294 | 1.00 | 39.27 |
| 562 | CA | TYR | A | 416 | 27.222 | 53.291 | 10.450 | 1.00 | 37.37 |
| 563 | CB | TYR | A | 416 | 27.172 | 54.498 | 9.519 | 1.00 | 33.61 |
| 564 | CG | TYR | A | 416 | 25.967 | 54.536 | 8.622 | 1.00 | 27.57 |
| 565 | CD1 | TYR | A | 416 | 24.706 | 54.847 | 9.126 | 1.00 | 26.31 |
| 566 | CE1 | TYR | A | 416 | 23.591 | 54.848 | 8.300 | 1.00 | 35.08 |
| 567 | CD2 | TYR | A | 416 | 26.084 | 54.229 | 7.275 | 1.00 | 28.68 |
| 568 | CE2 | TYR | A | 416 | 24.985 | 54.221 | 6.449 | 1.00 | 30.56 |
| 569 | CZ | TYR | A | 416 | 23.743 | 54.530 | 6.960 | 1.00 | 35.62 |
| 570 | OH | TYR | A | 416 | 22.656 | 54.512 | 6.116 | 1.00 | 44.94 |
| 571 | C | TYR | A | 416 | 28.494 | 53.344 | 11.267 | 1.00 | 38.31 |
| 572 | O | TYR | A | 416 | 28.508 | 53.856 | 12.391 | 1.00 | 37.29 |
| 573 | N | GLN | A | 417 | 29.569 | 52.819 | 10.684 | 1.00 | 39.74 |
| 574 | CA | GLN | A | 417 | 30.849 | 52.777 | 11.364 | 1.00 | 40.75 |
| 575 | CB | GLN | A | 417 | 31.143 | 51.346 | 11.836 | 1.00 | 41.60 |
| 576 | CG | GLN | A | 417 | 32.360 | 51.247 | 12.751 | 1.00 | 53.57 |
| 577 | CD | GLN | A | 417 | 32.603 | 49.838 | 13.271 | 1.00 | 65.04 |
| 578 | OE1 | GLN | A | 417 | 33.476 | 49.119 | 12.775 | 1.00 | 69.74 |
| 579 | NE2 | GLN | A | 417 | 31.823 | 49.435 | 14.269 | 1.00 | 66.91 |
| 580 | C | GLN | A | 417 | 32.022 | 53.280 | 10.535 | 1.00 | 39.48 |
| 581 | O | GLN | A | 417 | 32.149 | 52.981 | 9.346 | 1.00 | 40.03 |
| 582 | N | CYS | A | 418 | 32.882 | 54.043 | 11.188 | 1.00 | 39.47 |
| 583 | CA | CYS | A | 418 | 34.075 | 54.568 | 10.559 | 1.00 | 41.39 |
| 584 | C | CYS | A | 418 | 35.246 | 53.868 | 11.232 | 1.00 | 41.00 |
| 585 | O | CYS | A | 418 | 35.395 | 53.939 | 12.452 | 1.00 | 41.94 |
| 586 | CB | CYS | A | 418 | 34.187 | 56.087 | 10.781 | 1.00 | 39.12 |
| 587 | SG | CYS | A | 418 | 35.688 | 56.818 | 10.050 | 1.00 | 53.87 |
| 588 | N | ALA | A | 419 | 36.059 | 53.171 | 10.446 | 1.00 | 42.75 |
| 589 | CA | ALA | A | 419 | 37.238 | 52.498 | 10.982 | 1.00 | 45.21 |
| 590 | CB | ALA | A | 419 | 37.357 | 51.073 | 10.412 | 1.00 | 45.00 |
| 591 | C | ALA | A | 419 | 38.462 | 53.335 | 10.602 | 1.00 | 46.28 |
| 592 | O | ALA | A | 419 | 38.826 | 53.428 | 9.429 | 1.00 | 44.60 |
| 593 | N | VAL | A | 420 | 39.088 | 53.961 | 11.592 | 1.00 | 49.65 |
| 594 | CA | VAL | A | 420 | 40.252 | 54.789 | 11.322 | 1.00 | 54.91 |
| 595 | CB | VAL | A | 420 | 40.295 | 56.016 | 12.245 | 1.00 | 52.57 |
| 596 | CG1 | VAL | A | 420 | 41.515 | 56.858 | 11.911 | 1.00 | 54.41 |
| 597 | CG2 | VAL | A | 420 | 39.032 | 56.844 | 12.083 | 1.00 | 52.89 |
| 598 | C | VAL | A | 420 | 41.564 | 54.028 | 11.477 | 1.00 | 59.75 |
| 599 | O | VAL | A | 420 | 41.877 | 53.517 | 12.556 | 1.00 | 56.32 |
| 600 | N | THR | A | 421 | 42.325 | 53.960 | 10.387 | 1.00 | 65.72 |
| 601 | CA | THR | A | 421 | 43.615 | 53.278 | 10.383 | 1.00 | 72.95 |
| 602 | CB | THR | A | 421 | 43.733 | 52.297 | 9.197 | 1.00 | 73.73 |
| 603 | OG1 | THR | A | 421 | 42.702 | 51.306 | 9.282 | 1.00 | 78.26 |
| 604 | CG2 | THR | A | 421 | 45.082 | 51.605 | 9.217 | 1.00 | 74.84 |
| 605 | C | THR | A | 421 | 44.741 | 54.298 | 10.269 | 1.00 | 76.77 |
| 606 | O | THR | A | 421 | 45.141 | 54.663 | 9.164 | 1.00 | 76.76 |
| 607 | N | ALA | A | 422 | 45.248 | 54.757 | 11.410 | 1.00 | 81.68 |
| 608 | CA | ALA | A | 422 | 46.330 | 55.738 | 11.427 | 1.00 | 86.81 |
| 609 | CB | ALA | A | 422 | 46.358 | 56.466 | 12.768 | 1.00 | 86.67 |
| 610 | C | ALA | A | 422 | 47.670 | 55.060 | 11.178 | 1.00 | 90.55 |
| 611 | O | ALA | A | 422 | 47.843 | 53.876 | 11.477 | 1.00 | 91.00 |
| 612 | N | PRO | A | 423 | 48.641 | 55.807 | 10.625 | 1.00 | 93.77 |
| 613 | CD | PRO | A | 423 | 48.615 | 57.260 | 10.385 | 1.00 | 94.85 |
| 614 | CA | PRO | A | 423 | 49.970 | 55.257 | 10.341 | 1.00 | 95.83 |
| 615 | CB | PRO | A | 423 | 50.731 | 56.459 | 9.791 | 1.00 | 96.70 |
| 616 | CG | PRO | A | 423 | 50.074 | 57.619 | 10.487 | 1.00 | 97.10 |
| 617 | C | PRO | A | 423 | 50.612 | 54.674 | 11.596 | 1.00 | 97.16 |
| 618 | O | PRO | A | 423 | 50.850 | 53.472 | 11.674 | 1.00 | 97.34 |
| 619 | N | ALA | A | 424 | 50.893 | 55.524 | 12.578 | 1.00 | 98.50 |
| 620 | CA | ALA | A | 424 | 51.486 | 55.063 | 13.829 | 1.00 | 99.73 |
| 621 | CB | ALA | A | 424 | 51.995 | 56.252 | 14.626 | 1.00 | 99.11 |
| 622 | C | ALA | A | 424 | 50.412 | 54.318 | 14.625 | 1.00 | 100.69 |
| 623 | O | ALA | A | 424 | 49.503 | 53.725 | 14.046 | 1.00 | 101.64 |
| 624 | N | LEU | A | 425 | 50.521 | 54.355 | 15.950 | 1.00 | 100.65 |
| 625 | CA | LEU | A | 425 | 49.550 | 53.705 | 16.828 | 1.00 | 99.91 |
| 626 | CB | LEU | A | 425 | 48.182 | 54.372 | 16.671 | 1.00 | 100.27 |
| 627 | CG | LEU | A | 425 | 48.126 | 55.833 | 17.125 | 1.00 | 102.88 |
| 628 | CD1 | LEU | A | 425 | 46.735 | 56.386 | 16.889 | 1.00 | 102.68 |
| 629 | CD2 | LEU | A | 425 | 48.498 | 55.932 | 18.603 | 1.00 | 105.00 |
| 630 | C | LEU | A | 425 | 49.427 | 52.200 | 16.586 | 1.00 | 98.82 |
| 631 | O | LEU | A | 425 | 49.985 | 51.676 | 15.629 | 1.00 | 98.87 |
| 632 | N | PRO | A | 426 | 48.712 | 51.485 | 17.475 | 1.00 | 97.69 |
| 633 | CD | PRO | A | 426 | 48.418 | 51.939 | 18.849 | 1.00 | 98.02 |
| 634 | CA | PRO | A | 426 | 48.513 | 50.034 | 17.361 | 1.00 | 95.83 |
| 635 | CB | PRO | A | 426 | 48.673 | 49.570 | 18.797 | 1.00 | 96.48 |
| 636 | CG | PRO | A | 426 | 47.965 | 50.659 | 19.545 | 1.00 | 96.97 |
| 637 | C | PRO | A | 426 | 47.145 | 49.651 | 16.791 | 1.00 | 94.21 |
| 638 | O | PRO | A | 426 | 47.006 | 49.360 | 15.602 | 1.00 | 94.03 |
| 639 | N | ARG | A | 427 | 46.140 | 49.637 | 17.661 | 1.00 | 92.33 |
| 640 | CA | ARG | A | 427 | 44.778 | 49.297 | 17.269 | 1.00 | 90.72 |
| 641 | CB | ARG | A | 427 | 43.928 | 49.017 | 18.510 | 1.00 | 92.36 |
| 642 | CG | ARG | A | 427 | 44.349 | 47.816 | 19.342 | 1.00 | 96.36 |
| 643 | CD | ARG | A | 427 | 43.525 | 46.585 | 18.998 | 1.00 | 99.46 |
| 644 | NE | ARG | A | 427 | 43.247 | 45.778 | 20.184 | 1.00 | 101.30 |
| 645 | CZ | ARG | A | 427 | 42.540 | 46.203 | 21.228 | 1.00 | 102.28 |
| 646 | NE1 | ARG | A | 427 | 42.037 | 47.430 | 21.236 | 1.00 | 102.89 |
| 647 | NH2 | ARG | A | 427 | 42.334 | 45.400 | 22.264 | 1.00 | 102.96 |
| 648 | C | ARG | A | 427 | 44.152 | 50.458 | 16.505 | 1.00 | 88.67 |
| 649 | O | ARG | A | 427 | 44.292 | 51.614 | 16.902 | 1.00 | 88.90 |
| 650 | N | ALA | A | 428 | 43.462 | 50.153 | 15.413 | 1.00 | 85.79 |
| 651 | CA | ALA | A | 428 | 42.807 | 51.188 | 14.631 | 1.00 | 82.45 |
| 652 | CB | ALA | A | 428 | 42.310 | 50.615 | 13.311 | 1.00 | 82.49 |
| 653 | C | ALA | A | 428 | 41.637 | 51.707 | 15.456 | 1.00 | 79.74 |
| 654 | O | ALA | A | 428 | 41.032 | 50.955 | 16.221 | 1.00 | 79.87 |
| 655 | N | LEU | A | 429 | 41.330 | 52.994 | 15.315 | 1.00 | 76.91 |
| 656 | CA | LEU | A | 429 | 40.223 | 53.604 | 16.049 | 1.00 | 72.60 |
| 657 | CB | LEU | A | 429 | 40.405 | 55.125 | 16.126 | 1.00 | 73.30 |
| 658 | CG | LEU | A | 429 | 41.684 | 55.685 | 16.757 | 1.00 | 75.14 |
| 659 | CD1 | LEU | A | 429 | 41.706 | 57.197 | 16.600 | 1.00 | 70.88 |
| 660 | CD2 | LEU | A | 429 | 41.756 | 55.296 | 18.227 | 1.00 | 79.00 |
| 661 | C | LEU | A | 429 | 38.921 | 53.290 | 15.323 | 1.00 | 69.58 |
| 662 | O | LEU | A | 429 | 38.894 | 53.209 | 14.094 | 1.00 | 67.68 |
| 663 | N | MET | A | 430 | 37.845 | 53.116 | 16.085 | 1.00 | 66.58 |
| 664 | CA | MET | A | 430 | 36.543 | 52.815 | 15.504 | 1.00 | 63.72 |
| 665 | CB | MET | A | 430 | 36.203 | 51.340 | 15.704 | 1.00 | 66.69 |
| 666 | CG | MET | A | 430 | 37.237 | 50.395 | 15.125 | 1.00 | 76.81 |
| 667 | SD | MET | A | 430 | 36.764 | 48.668 | 15.345 | 1.00 | 89.36 |
| 668 | CE | MET | A | 430 | 36.990 | 48.471 | 17.110 | 1.00 | 87.45 |
| 669 | C | MET | A | 430 | 35.450 | 53.674 | 16.119 | 1.00 | 59.15 |
| 670 | O | MET | A | 430 | 35.371 | 53.822 | 17.340 | 1.00 | 59.09 |
| 671 | N | ARG | A | 431 | 34.607 | 54.245 | 15.267 | 1.00 | 55.24 |
| 672 | CA | ARG | A | 431 | 33.523 | 55.091 | 15.746 | 1.00 | 51.07 |
| 673 | CB | ARG | A | 431 | 33.863 | 56.564 | 15.516 | 1.00 | 51.17 |
| 674 | CG | ARG | A | 431 | 35.291 | 56.939 | 15.897 | 1.00 | 55.59 |
| 675 | CD | ARG | A | 431 | 35.320 | 58.159 | 16.805 | 1.00 | 67.55 |
| 676 | NE | ARG | A | 431 | 35.147 | 57.816 | 18.216 | 1.00 | 74.62 |
| 677 | CZ | ARG | A | 431 | 36.128 | 57.380 | 19.001 | 1.00 | 76.90 |
| 678 | NH1 | ARG | A | 431 | 37.353 | 57.236 | 18.514 | 1.00 | 77.02 |
| 679 | NH2 | ARG | A | 431 | 35.888 | 57.087 | 20.274 | 1.00 | 79.64 |
| 680 | C | ARG | A | 431 | 32.244 | 54.732 | 15.013 | 1.00 | 47.90 |
| 681 | O | ARG | A | 431 | 32.277 | 54.386 | 13.832 | 1.00 | 46.26 |
| 682 | N | SER | A | 432 | 31.120 | 54.812 | 15.715 | 1.00 | 45.54 |
| 683 | CA | SER | A | 432 | 29.832 | 54.495 | 15.112 | 1.00 | 45.94 |
| 684 | CB | SER | A | 432 | 29.356 | 53.116 | 15.584 | 1.00 | 47.12 |
| 685 | OG | SER | A | 432 | 29.331 | 53.040 | 16.994 | 1.00 | 50.92 |
| 686 | C | SER | A | 432 | 28.764 | 55.537 | 15.412 | 1.00 | 43.27 |
| 687 | O | SER | A | 432 | 28.862 | 56.272 | 16.391 | 1.00 | 42.70 |
| 688 | N | THR | A | 433 | 27.736 | 55.588 | 14.569 | 1.00 | 40.97 |
| 689 | CA | THR | A | 433 | 26.663 | 56.552 | 14.758 | 1.00 | 38.90 |
| 690 | CB | THR | A | 433 | 26.967 | 57.875 | 14.027 | 1.00 | 39.50 |
| 691 | OG1 | THR | A | 433 | 25.911 | 58.808 | 14.280 | 1.00 | 38.91 |
| 692 | CG2 | THR | A | 433 | 27.093 | 57.646 | 12.516 | 1.00 | 36.51 |
| 693 | C | THR | A | 433 | 25.329 | 56.017 | 14.262 | 1.00 | 39.62 |
| 694 | O | THR | A | 433 | 25.283 | 55.188 | 13.356 | 1.00 | 39.87 |
| 695 | N | THR | A | 434 | 24.249 | 56.495 | 14.876 | 1.00 | 39.10 |
| 696 | CA | THR | A | 434 | 22.889 | 56.101 | 14.531 | 1.00 | 39.67 |
| 697 | CB | THR | A | 434 | 22.480 | 54.812 | 15.282 | 1.00 | 43.40 |
| 698 | OG1 | THR | A | 434 | 22.307 | 55.104 | 16.675 | 1.00 | 53.59 |
| 699 | CG2 | THR | A | 434 | 23.559 | 53.770 | 15.171 | 1.00 | 52.29 |
| 700 | C | THR | A | 434 | 21.939 | 57.220 | 14.969 | 1.00 | 38.41 |
| 701 | O | THR | A | 434 | 22.325 | 58.110 | 15.726 | 1.00 | 38.84 |
| 702 | N | ALA | A | 435 | 20.701 | 57.179 | 14.506 | 1.00 | 37.73 |
| 703 | CA | ALA | A | 435 | 19.747 | 58.190 | 14.911 | 1.00 | 44.38 |
| 704 | CB | ALA | A | 435 | 18.426 | 57.968 | 14.213 | 1.00 | 40.72 |
| 705 | C | ALA | A | 435 | 19.577 | 58.033 | 16.421 | 1.00 | 47.78 |
| 706 | O | ALA | A | 435 | 19.611 | 56.919 | 16.937 | 1.00 | 46.98 |
| 707 | N | THR | A | 436 | 19.413 | 59.146 | 17.129 | 1.00 | 52.31 |
| 708 | CA | THR | A | 436 | 19.217 | 59.094 | 18.574 | 1.00 | 55.83 |
| 709 | CB | THR | A | 436 | 19.492 | 60.469 | 19.240 | 1.00 | 59.83 |
| 710 | OG1 | THR | A | 436 | 20.873 | 60.816 | 19.072 | 1.00 | 62.64 |
| 711 | CG2 | THR | A | 436 | 19.159 | 60.425 | 20.732 | 1.00 | 60.56 |
| 712 | C | THR | A | 436 | 17.766 | 58.707 | 18.840 | 1.00 | 56.91 |
| 713 | O | THR | A | 436 | 16.852 | 59.242 | 18.212 | 1.00 | 55.51 |
| 714 | N | SER | A | 437 | 17.557 | 57.759 | 19.748 | 1.00 | 58.87 |
| 715 | CA | SER | A | 437 | 16.202 | 57.344 | 20.095 | 1.00 | 61.80 |
| 716 | CB | SER | A | 437 | 16.155 | 55.854 | 20.474 | 1.00 | 63.69 |
| 717 | OG | SER | A | 437 | 16.984 | 55.571 | 21.589 | 1.00 | 67.24 |
| 718 | C | SER | A | 437 | 15.753 | 58.203 | 21.272 | 1.00 | 61.35 |
| 719 | O | SER | A | 437 | 16.477 | 59.111 | 21.690 | 1.00 | 61.92 |
| 720 | N | GLY | A | 438 | 14.567 | 57.930 | 21.803 | 1.00 | 60.43 |
| 721 | CA | GLY | A | 438 | 14.087 | 58.719 | 22.923 | 1.00 | 59.27 |
| 722 | C | GLY | A | 438 | 12.999 | 59.691 | 22.510 | 1.00 | 58.25 |
| 723 | O | GLY | A | 438 | 12.739 | 59.847 | 21.321 | 1.00 | 58.13 |
| 724 | N | PRO | A | 439 | 12.347 | 60.363 | 23.472 | 1.00 | 58.84 |
| 725 | CD | PRO | A | 439 | 12.555 | 60.215 | 24.923 | 1.00 | 60.09 |
| 726 | CA | PRO | A | 439 | 11.275 | 61.323 | 23.203 | 1.00 | 57.04 |
| 727 | CB | PRO | A | 439 | 10.908 | 61.832 | 24.597 | 1.00 | 57.59 |
| 728 | CG | PRO | A | 439 | 11.224 | 60.667 | 25.472 | 1.00 | 59.40 |
| 729 | C | PRO | A | 439 | 11.691 | 62.453 | 22.272 | 1.00 | 54.66 |
| 730 | O | PRO | A | 439 | 12.877 | 62.776 | 22.155 | 1.00 | 56.08 |
| 731 | N | ARG | A | 440 | 10.703 | 63.052 | 21.618 | 1.00 | 51.95 |
| 732 | CA | ARG | A | 440 | 10.942 | 64.149 | 20.695 | 1.00 | 49.50 |
| 733 | CB | ARG | A | 440 | 10.471 | 63.771 | 19.283 | 1.00 | 51.89 |
| 734 | CG | ARG | A | 440 | 10.921 | 62.400 | 18.791 | 1.00 | 59.84 |
| 735 | CD | ARG | A | 440 | 12.412 | 62.334 | 18.465 | 1.00 | 66.22 |
| 736 | NE | ARG | A | 440 | 12.845 | 60.943 | 18.313 | 1.00 | 74.61 |
| 737 | CZ | ARG | A | 440 | 14.047 | 60.555 | 17.896 | 1.00 | 76.10 |
| 738 | NH1 | ARG | A | 440 | 14.967 | 61.444 | 17.573 | 1.00 | 77.20 |
| 739 | NH2 | ARG | A | 440 | 14.328 | 59.262 | 17.805 | 1.00 | 84.96 |
| 740 | C | ARG | A | 440 | 10.145 | 65.351 | 21.190 | 1.00 | 46.80 |
| 741 | O | ARG | A | 440 | 9.058 | 65.194 | 21.746 | 1.00 | 46.09 |
| 742 | N | ALA | A | 441 | 10.696 | 66.547 | 21.001 | 1.00 | 42.67 |
| 743 | CA | ALA | A | 441 | 10.024 | 67.779 | 21.402 | 1.00 | 39.13 |
| 744 | CB | ALA | A | 441 | 10.384 | 68.142 | 22.837 | 1.00 | 32.25 |
| 745 | C | ALA | A | 441 | 10.452 | 68.888 | 20.454 | 1.00 | 39.00 |
| 746 | O | ALA | A | 441 | 11.639 | 69.045 | 20.175 | 1.00 | 38.16 |
| 747 | N | ALA | A | 442 | 9.482 | 69.651 | 19.964 | 1.00 | 37.20 |
| 748 | CA | ALA | A | 442 | 9.736 | 70.739 | 19.037 | 1.00 | 37.26 |
| 749 | CB | ALA | A | 442 | 8.413 | 71.288 | 18.526 | 1.00 | 37.47 |
| 750 | C | ALA | A | 442 | 10.568 | 71.882 | 19.617 | 1.00 | 39.85 |
| 751 | O | ALA | A | 442 | 10.612 | 72.090 | 20.825 | 1.00 | 41.57 |
| 752 | N | PRO | A | 443 | 11.246 | 72.640 | 18.743 | 1.00 | 39.83 |
| 753 | CD | PRO | A | 443 | 11.478 | 72.297 | 17.334 | 1.00 | 40.96 |
| 754 | CA | PRO | A | 443 | 12.080 | 73.775 | 19.131 | 1.00 | 38.29 |
| 755 | CB | PRO | A | 443 | 13.058 | 73.922 | 17.956 | 1.00 | 36.98 |
| 756 | CG | PRO | A | 443 | 12.907 | 72.675 | 17.168 | 1.00 | 36.14 |
| 757 | C | PRO | A | 443 | 11.220 | 75.037 | 19.272 | 1.00 | 40.19 |
| 758 | O | PRO | A | 443 | 10.204 | 75.194 | 18.588 | 1.00 | 40.51 |
| 759 | N | ALA | A | 444 | 11.636 | 75.927 | 20.163 | 1.00 | 39.07 |
| 760 | CA | ALA | A | 444 | 10.942 | 77.188 | 20.376 | 1.00 | 36.58 |
| 761 | CD | ALA | A | 444 | 10.605 | 77.372 | 21.860 | 1.00 | 40.22 |
| 762 | C | ALA | A | 444 | 11.962 | 78.227 | 19.919 | 1.00 | 34.33 |
| 763 | O | ALA | A | 444 | 13.109 | 78.242 | 20.398 | 1.00 | 30.65 |
| 764 | N | VAL | A | 445 | 11.543 | 79.081 | 18.991 | 1.00 | 28.37 |
| 765 | CA | VAL | A | 445 | 12.428 | 80.080 | 18.436 | 1.00 | 26.39 |
| 766 | GB | VAL | A | 445 | 12.448 | 79.948 | 16.902 | 1.00 | 26.69 |
| 767 | CG1 | VAL | A | 445 | 13.395 | 80.980 | 16.310 | 1.00 | 23.97 |
| 768 | CG2 | VAL | A | 445 | 12.853 | 78.524 | 16.514 | 1.00 | 22.56 |
| 769 | C | VAL | A | 445 | 12.103 | 81.532 | 18.809 | 1.00 | 27.26 |
| 770 | O | VAL | A | 445 | 10.956 | 81.954 | 18.733 | 1.00 | 24.12 |
| 771 | N | TYR | A | 446 | 13.127 | 82.281 | 19.207 | 1.00 | 23.45 |
| 772 | CA | TYR | A | 446 | 12.955 | 83.681 | 19.559 | 1.00 | 25.54 |
| 773 | CB | TYR | A | 446 | 12.868 | 83.878 | 21.085 | 1.00 | 24.97 |
| 774 | CC | TYR | A | 446 | 12.704 | 85.336 | 21.440 | 1.00 | 24.95 |
| 775 | CD1 | TYR | A | 446 | 11.639 | 86.083 | 20.918 | 1.00 | 37.39 |
| 776 | CE1 | TYR | A | 446 | 11.506 | 87.454 | 21.193 | 1.00 | 34.09 |
| 777 | CD2 | TYR | A | 446 | 13.627 | 85.987 | 22.249 | 1.00 | 28.20 |
| 778 | CE2 | TYR | A | 446 | 13.508 | 87.355 | 22.533 | 1.00 | 31.02 |
| 779 | CZ | TYR | A | 446 | 12.440 | 88.081 | 21.998 | 1.00 | 34.30 |
| 780 | OH | TYR | A | 446 | 12.306 | 89.426 | 22.254 | 1.00 | 35.42 |
| 781 | C | TYR | A | 446 | 14.135 | 84.470 | 19.012 | 1.00 | 23.87 |
| 782 | O | TYR | A | 446 | 15.275 | 84.279 | 19.437 | 1.00 | 28.38 |
| 783 | N | ALA | A | 447 | 13.858 | 85.346 | 18.058 | 1.00 | 26.40 |
| 784 | CA | ALA | A | 447 | 14.893 | 86.155 | 17.429 | 1.00 | 27.30 |
| 785 | CB | ALA | A | 447 | 14.766 | 86.065 | 15.905 | 1.00 | 24.55 |
| 786 | C | ALA | A | 447 | 14.760 | 87.598 | 17.898 | 1.00 | 30.87 |
| 787 | O | ALA | A | 447 | 13.655 | 88.085 | 18.121 | 1.00 | 32.42 |
| 788 | N | PHE | A | 448 | 15.880 | 88.290 | 18.055 | 1.00 | 31.33 |
| 789 | CA | PHE | A | 448 | 15.801 | 89.661 | 18.518 | 1.00 | 34.25 |
| 790 | CB | PHE | A | 448 | 15.616 | 89.662 | 20.040 | 1.00 | 40.48 |
| 791 | CG | PHE | A | 448 | 16.795 | 89.103 | 20.786 | 1.00 | 39.26 |
| 792 | CD1 | PHE | A | 448 | 17.837 | 89.937 | 21.177 | 1.00 | 39.27 |
| 793 | CD2 | PHE | A | 448 | 16.896 | 87.738 | 21.041 | 1.00 | 36.97 |
| 794 | CE1 | PHE | A | 448 | 18.967 | 89.420 | 21.814 | 1.00 | 43.41 |
| 795 | CE2 | PHE | A | 448 | 18.022 | 87.211 | 21.678 | 1.00 | 36.78 |
| 796 | CZ | PHE | A | 448 | 19.059 | 88.054 | 22.064 | 1.00 | 37.14 |
| 797 | C | PHE | A | 448 | 17.021 | 90.487 | 18.148 | 1.00 | 34.75 |
| 798 | O | PHE | A | 448 | 18.046 | 89.954 | 17.731 | 1.00 | 34.97 |
| 799 | N | ALA | A | 449 | 16.892 | 91.799 | 18.302 | 1.00 | 37.02 |
| 800 | CA | ALA | A | 449 | 17.977 | 92.723 | 18.021 | 1.00 | 38.65 |
| 801 | CB | ALA | A | 449 | 17.478 | 93.869 | 17.202 | 1.00 | 32.34 |
| 802 | C | ALA | A | 449 | 18.550 | 93.250 | 19.333 | 1.00 | 42.92 |
| 803 | O | ALA | A | 449 | 17.826 | 93.435 | 20.306 | 1.00 | 42.07 |
| 804 | N | THR | A | 450 | 19.857 | 93.489 | 19.341 | 1.00 | 47.71 |
| 805 | CA | THR | A | 450 | 20.552 | 94.014 | 20.503 | 1.00 | 52.16 |
| 806 | CB | THR | A | 450 | 22.023 | 93.560 | 20.501 | 1.00 | 53.90 |
| 807 | OG1 | THR | A | 450 | 22.078 | 92.132 | 20.415 | 1.00 | 61.82 |
| 808 | CG2 | THR | A | 450 | 22.723 | 94.006 | 21.773 | 1.00 | 62.58 |
| 809 | C | THR | A | 450 | 20.503 | 95.547 | 20.493 | 1.00 | 54.69 |
| 810 | O | THR | A | 450 | 20.617 | 96.179 | 19.441 | 1.00 | 51.28 |
| 811 | N | PRO | A | 451 | 20.320 | 96.161 | 21.669 | 1.00 | 57.24 |
| 812 | CD | PRO | A | 451 | 19.982 | 95.539 | 22.963 | 1.00 | 60.66 |
| 813 | CA | PRO | A | 451 | 20.261 | 97.623 | 21.762 | 1.00 | 61.04 |
| 814 | CB | PRO | A | 451 | 20.029 | 97.864 | 23.254 | 1.00 | 61.38 |
| 815 | CG | PRO | A | 451 | 19.240 | 96.654 | 23.667 | 1.00 | 63.63 |
| 816 | C | PRO | A | 451 | 21.537 | 98.299 | 21.253 | 1.00 | 63.11 |
| 817 | O | PRO | A | 451 | 22.651 | 97.918 | 21.616 | 1.00 | 61.66 |
| 818 | N | GLU | A | 452 | 21.370 | 99.297 | 20.394 | 1.00 | 66.04 |
| 819 | CA | GLU | A | 452 | 22.517 | 100.025 | 19.863 | 1.00 | 69.40 |
| 820 | CB | GLU | A | 452 | 22.646 | 99.800 | 18.347 | 1.00 | 67.34 |
| 821 | CG | GLU | A | 452 | 24.095 | 99.689 | 17.823 | 1.00 | 60.86 |
| 822 | CD | GLU | A | 452 | 24.785 | 98.375 | 18.224 | 1.00 | 59.08 |
| 823 | OE1 | GLU | A | 452 | 24.090 | 97.345 | 18.325 | 1.00 | 62.62 |
| 824 | OE2 | GLU | A | 452 | 26.022 | 98.351 | 18.424 | 1.00 | 45.82 |
| 825 | C | GLU | A | 452 | 22.322 | 101.510 | 20.170 | 1.00 | 71.98 |
| 826 | O | GLU | A | 452 | 21.705 | 102.240 | 19.395 | 1.00 | 70.35 |
| 827 | N | ALA | A | 453 | 22.842 | 101.941 | 21.317 | 1.00 | 75.33 |
| 828 | CA | ALA | A | 453 | 22.733 | 103.334 | 21.753 | 1.00 | 78.80 |
| 829 | CB | ALA | A | 453 | 23.482 | 103.525 | 23.071 | 1.00 | 80.74 |
| 830 | C | ALA | A | 453 | 23.260 | 104.318 | 20.706 | 1.00 | 80.53 |
| 831 | O | ALA | A | 453 | 24.167 | 104.001 | 19.933 | 1.00 | 82.95 |
| 832 | N | LYS | A | 459 | 29.802 | 96.915 | 16.453 | 1.00 | 51.87 |
| 833 | CA | LYS | A | 459 | 28.953 | 96.350 | 15.407 | 1.00 | 52.84 |
| 834 | CB | LYS | A | 459 | 29.613 | 95.121 | 14.783 | 1.00 | 55.20 |
| 835 | CG | LYS | A | 459 | 30.879 | 95.429 | 14.011 | 1.00 | 63.98 |
| 836 | CD | LYS | A | 459 | 31.199 | 94.329 | 13.012 | 1.00 | 72.67 |
| 837 | CE | LYS | A | 459 | 30.116 | 94.234 | 11.943 | 1.00 | 77.63 |
| 838 | NZ | LYS | A | 459 | 30.478 | 93.298 | 10.841 | 1.00 | 83.78 |
| 839 | C | LYS | A | 459 | 27.567 | 95.962 | 15.905 | 1.00 | 50.86 |
| 840 | O | LYS | A | 459 | 27.426 | 95.389 | 16.982 | 1.00 | 49.65 |
| 841 | N | ARG | A | 460 | 26.555 | 96.268 | 15.099 | 1.00 | 48.30 |
| 842 | CA | ARG | A | 460 | 25.167 | 95.960 | 15.426 | 1.00 | 47.61 |
| 843 | CB | ARG | A | 460 | 24.250 | 96.735 | 14.480 | 1.00 | 51.65 |
| 844 | CG | ARG | A | 460 | 24.571 | 98.229 | 14.509 | 1.00 | 59.85 |
| 845 | CD | ARG | A | 460 | 23.816 | 99.030 | 13.465 | 1.00 | 65.41 |
| 846 | NE | ARG | A | 460 | 24.200 | 100.442 | 13.497 | 1.00 | 70.36 |
| 847 | CZ | ARG | A | 460 | 25.426 | 100.898 | 13.252 | 1.00 | 73.06 |
| 848 | NH1 | ARG | A | 460 | 26.412 | 100.060 | 12.950 | 1.00 | 75.61 |
| 849 | NH2 | ARG | A | 460 | 25.671 | 102.199 | 13.309 | 1.00 | 76.52 |
| 850 | C | ARG | A | 460 | 24.936 | 94.446 | 15.341 | 1.00 | 44.39 |
| 851 | O | ARG | A | 460 | 25.542 | 93.761 | 14.511 | 1.00 | 41.11 |
| 852 | N | THR | A | 461 | 24.060 | 93.928 | 16.195 | 1.00 | 40.18 |
| 853 | CA | THR | A | 461 | 23.832 | 92.489 | 16.250 | 1.00 | 40.02 |
| 854 | CB | THR | A | 461 | 24.615 | 91.867 | 17.441 | 1.00 | 41.84 |
| 855 | OG1 | THR | A | 461 | 25.999 | 92.217 | 17.350 | 1.00 | 52.98 |
| 856 | CG2 | THR | A | 461 | 24.498 | 90.362 | 17.434 | 1.00 | 48.91 |
| 857 | C | THR | A | 461 | 22.393 | 92.026 | 16.401 | 1.00 | 37.19 |
| 858 | O | THR | A | 461 | 21.591 | 92.641 | 17.118 | 1.00 | 37.94 |
| 859 | N | LEU | A | 462 | 22.088 | 90.919 | 15.725 | 1.00 | 33.00 |
| 860 | CA | LEU | A | 462 | 20.791 | 90.265 | 15.811 | 1.00 | 29.46 |
| 861 | CB | LEU | A | 462 | 20.166 | 90.050 | 14.433 | 1.00 | 30.35 |
| 862 | CG | LEU | A | 462 | 19.922 | 91.318 | 13.602 | 1.00 | 33.09 |
| 863 | CD1 | LEU | A | 462 | 19.188 | 90.961 | 12.315 | 1.00 | 32.32 |
| 864 | CD2 | LEU | A | 462 | 19.101 | 92.307 | 14.401 | 1.00 | 25.52 |
| 865 | C | LEU | A | 462 | 21.161 | 88.928 | 16.438 | 1.00 | 29.44 |
| 866 | O | LEU | A | 462 | 22.261 | 88.416 | 16.206 | 1.00 | 24.62 |
| 867 | N | ALA | A | 463 | 20.257 | 88.363 | 17.232 | 1.00 | 27.23 |
| 868 | CA | ALA | A | 463 | 20.550 | 87.114 | 17.908 | 1.00 | 27.78 |
| 869 | CB | ALA | A | 463 | 21.112 | 87.401 | 19.310 | 1.00 | 26.61 |
| 870 | C | ALA | A | 463 | 19.299 | 86.268 | 17.996 | 1.00 | 28.37 |
| 871 | O | ALA | A | 463 | 18.185 | 86.776 | 17.867 | 1.00 | 29.09 |
| 872 | N | CYS | A | 464 | 19.485 | 84.977 | 18.230 | 1.00 | 22.40 |
| 873 | CA | CYS | A | 464 | 18.354 | 84.066 | 18.279 | 1.00 | 26.72 |
| 874 | C | CYS | A | 464 | 18.562 | 82.978 | 19.314 | 1.00 | 24.16 |
| 875 | O | CYS | A | 464 | 19.648 | 82.401 | 19.409 | 1.00 | 23.01 |
| 876 | CB | CYS | A | 464 | 18.190 | 83.428 | 16.900 | 1.00 | 24.58 |
| 877 | SG | CYS | A | 464 | 16.806 | 82.273 | 16.677 | 1.00 | 40.31 |
| 878 | N | LEU | A | 465 | 17.504 | 82.692 | 20.068 | 1.00 | 22.68 |
| 879 | CA | LEU | A | 465 | 17.548 | 81.650 | 21.087 | 1.00 | 24.31 |
| 880 | CB | LEU | A | 465 | 17.127 | 82.210 | 22.460 | 1.00 | 26.81 |
| 881 | CG | LEU | A | 465 | 16.759 | 81.181 | 23.546 | 1.00 | 21.10 |
| 882 | CD1 | LEU | A | 465 | 17.941 | 80.290 | 23.849 | 1.00 | 21.73 |
| 883 | CD2 | LEU | A | 465 | 16.338 | 81.900 | 24.807 | 1.00 | 30.90 |
| 884 | C | LEU | A | 465 | 16.587 | 80.557 | 20.647 | 1.00 | 23.79 |
| 885 | O | LEU | A | 465 | 15.417 | 80.830 | 20.359 | 1.00 | 22.87 |
| 886 | N | ILE | A | 466 | 17.079 | 79.324 | 20.590 | 1.00 | 23.16 |
| 887 | CA | ILE | A | 466 | 16.251 | 78.198 | 20.174 | 1.00 | 23.50 |
| 888 | CB | ILE | A | 466 | 16.762 | 77.582 | 18.849 | 1.00 | 23.66 |
| 889 | CG2 | ILE | A | 466 | 15.800 | 76.460 | 18.395 | 1.00 | 29.47 |
| 890 | CG1 | ILE | A | 466 | 16.861 | 78.687 | 17.780 | 1.00 | 19.89 |
| 891 | CD1 | ILE | A | 466 | 17.371 | 78.202 | 16.450 | 1.00 | 36.91 |
| 892 | C | ILE | A | 466 | 16.326 | 77.212 | 21.316 | 1.00 | 22.24 |
| 893 | O | ILE | A | 466 | 17.411 | 76.789 | 21.715 | 1.00 | 24.74 |
| 894 | N | GLN | A | 467 | 15.167 | 76.811 | 21.830 | 1.00 | 23.91 |
| 895 | CA | GLN | A | 467 | 15.182 | 75.958 | 23.005 | 1.00 | 31.01 |
| 896 | CB | GLN | A | 467 | 15.224 | 76.853 | 24.254 | 1.00 | 30.11 |
| 897 | CG | GLN | A | 467 | 13.993 | 77.731 | 24.446 | 1.00 | 26.37 |
| 898 | CD | GLN | A | 467 | 14.114 | 78.680 | 25.636 | 1.00 | 29.83 |
| 899 | OE1 | GLN | A | 467 | 14.890 | 78.464 | 26.579 | 1.00 | 28.59 |
| 900 | NE2 | GLN | A | 467 | 13.318 | 79.731 | 25.605 | 1.00 | 31.49 |
| 901 | C | GLN | A | 467 | 14.054 | 74.959 | 23.148 | 1.00 | 33.72 |
| 902 | O | GLN | A | 467 | 13.131 | 74.909 | 22.332 | 1.00 | 31.53 |
| 903 | N | ASN | A | 468 | 14.179 | 74.158 | 24.205 | 1.00 | 38.27 |
| 904 | CA | ASN | A | 468 | 13.215 | 73.135 | 24.574 | 1.00 | 41.08 |
| 905 | CB | ASN | A | 468 | 11.856 | 73.761 | 24.855 | 1.00 | 43.70 |
| 906 | CG | ASN | A | 468 | 11.949 | 74.935 | 25.797 | 1.00 | 50.48 |
| 907 | OD1 | ASN | A | 468 | 12.641 | 74.878 | 26.819 | 1.00 | 59.45 |
| 908 | ND2 | ASN | A | 468 | 11.243 | 76.006 | 25.472 | 1.00 | 56.28 |
| 909 | C | ASN | A | 468 | 13.052 | 72.040 | 23.546 | 1.00 | 41.04 |
| 910 | O | ASN | A | 468 | 11.986 | 71.445 | 23.440 | 1.00 | 38.53 |
| 911 | N | PHE | A | 469 | 14.101 | 71.757 | 22.791 | 1.00 | 38.66 |
| 912 | CA | PHE | A | 469 | 13.996 | 70.706 | 21.797 | 1.00 | 38.07 |
| 913 | CB | PHE | A | 469 | 14.419 | 71.240 | 20.427 | 1.00 | 29.35 |
| 914 | CG | PHE | A | 469 | 15.888 | 71.536 | 20.309 | 1.00 | 28.48 |
| 915 | CD1 | PHE | A | 469 | 16.785 | 70.541 | 19.987 | 1.00 | 23.56 |
| 916 | CD2 | PHE | A | 469 | 16.369 | 72.823 | 20.521 | 1.00 | 24.16 |
| 917 | CE1 | PHE | A | 469 | 18.137 | 70.816 | 19.872 | 1.00 | 27.41 |
| 918 | CE2 | PHE | A | 469 | 17.733 | 73.107 | 20.409 | 1.00 | 32.69 |
| 919 | CZ | PHE | A | 469 | 18.616 | 72.094 | 20.083 | 1.00 | 24.40 |
| 920 | C | PHE | A | 469 | 14.815 | 69.475 | 22.161 | 1.00 | 38.11 |
| 921 | O | PHE | A | 469 | 15.848 | 69.574 | 22.813 | 1.00 | 39.11 |
| 922 | N | MET | A | 470 | 14.333 | 68.313 | 21.741 | 1.00 | 40.05 |
| 923 | CA | MET | A | 470 | 15.001 | 67.031 | 21.970 | 1.00 | 41.39 |
| 924 | CB | MET | A | 470 | 14.618 | 66.417 | 23.321 | 1.00 | 46.13 |
| 925 | CG | MET | A | 470 | 13.159 | 66.043 | 23.530 | 1.00 | 60.40 |
| 926 | SD | MET | A | 470 | 12.863 | 65.634 | 25.280 | 1.00 | 65.64 |
| 927 | CE | MET | A | 470 | 13.541 | 63.941 | 25.373 | 1.00 | 71.36 |
| 928 | C | MET | A | 470 | 14.505 | 66.183 | 20.820 | 1.00 | 38.04 |
| 929 | O | MET | A | 470 | 13.327 | 66.224 | 20.482 | 1.00 | 36.87 |
| 930 | N | PRO | A | 471 | 15.399 | 65.416 | 20.175 | 1.00 | 37.84 |
| 931 | CD | PRO | A | 471 | 14.946 | 64.687 | 18.983 | 1.00 | 38.40 |
| 932 | CA | PRO | A | 471 | 16.853 | 65.240 | 20.358 | 1.00 | 37.04 |
| 933 | CB | PRO | A | 471 | 17.200 | 64.115 | 19.368 | 1.00 | 38.82 |
| 934 | CG | PRO | A | 471 | 15.852 | 63.524 | 19.004 | 1.00 | 40.26 |
| 935 | C | PRO | A | 471 | 17.685 | 66.508 | 20.104 | 1.00 | 35.06 |
| 936 | O | PRO | A | 471 | 17.157 | 67.524 | 19.668 | 1.00 | 33.27 |
| 937 | N | GLU | A | 472 | 18.989 | 66.420 | 20.340 | 1.00 | 35.63 |
| 938 | CA | GLU | A | 472 | 19.895 | 67.567 | 20.202 | 1.00 | 38.83 |
| 939 | CB | GLU | A | 472 | 21.169 | 67.308 | 20.991 | 1.00 | 43.92 |
| 940 | CG | GLU | A | 472 | 22.084 | 66.301 | 20.308 | 1.00 | 54.98 |
| 941 | CD | GLU | A | 472 | 23.312 | 65.943 | 21.137 | 1.00 | 65.38 |
| 942 | OE1 | GLU | A | 472 | 23.431 | 66.428 | 22.279 | 1.00 | 73.96 |
| 943 | OE2 | GLU | A | 472 | 24.158 | 65.167 | 20.650 | 1.00 | 71.17 |
| 944 | C | GLU | A | 472 | 20.308 | 68.032 | 18.806 | 1.00 | 36.65 |
| 945 | O | GLU | A | 472 | 20.799 | 69.147 | 18.649 | 1.00 | 32.41 |
| 946 | N | ASP | A | 473 | 20.118 | 67.198 | 17.789 | 1.00 | 35.48 |
| 947 | CA | ASP | A | 473 | 20.508 | 67.596 | 16.432 | 1.00 | 34.00 |
| 948 | CB | ASP | A | 473 | 20.534 | 66.375 | 15.506 | 1.00 | 39.24 |
| 949 | CG | ASP | A | 473 | 21.607 | 65.361 | 15.907 | 1.00 | 48.86 |
| 950 | OD1 | ASP | A | 473 | 22.724 | 65.800 | 16.283 | 1.00 | 42.86 |
| 951 | OD2 | ASP | A | 473 | 21.330 | 64.139 | 15.835 | 1.00 | 46.87 |
| 952 | C | ASP | A | 473 | 19.607 | 68.683 | 15.844 | 1.00 | 34.33 |
| 953 | O | ASP | A | 473 | 18.380 | 68.543 | 15.785 | 1.00 | 34.18 |
| 954 | N | ILE | A | 474 | 20.216 | 69.776 | 15.409 | 1.00 | 27.93 |
| 955 | CA | ILE | A | 474 | 19.435 | 70.862 | 14.846 | 1.00 | 28.40 |
| 956 | CB | ILE | A | 474 | 18.874 | 71.752 | 15.971 | 1.00 | 25.16 |
| 957 | CG2 | ILE | A | 474 | 19.998 | 72.564 | 16.611 | 1.00 | 26.53 |
| 958 | CG1 | ILE | A | 474 | 17.772 | 72.665 | 15.436 | 1.00 | 21.56 |
| 959 | CD1 | ILE | A | 474 | 17.009 | 73.381 | 16.593 | 1.00 | 23.93 |
| 960 | C | ILE | A | 474 | 20.274 | 71.710 | 13.910 | 1.00 | 26.83 |
| 961 | O | ILE | A | 474 | 21.473 | 71.827 | 14.086 | 1.00 | 25.94 |
| 962 | N | SER | A | 475 | 19.625 | 72.275 | 12.898 | 1.00 | 24.54 |
| 963 | CA | SER | A | 475 | 20.264 | 73.154 | 11.933 | 1.00 | 23.71 |
| 964 | CB | SER | A | 475 | 20.014 | 72.668 | 10.510 | 1.00 | 22.00 |
| 965 | OG | SER | A | 475 | 20.555 | 71.379 | 10.341 | 1.00 | 30.00 |
| 966 | C | SER | A | 475 | 19.633 | 74.529 | 12.117 | 1.00 | 24.55 |
| 967 | O | SER | A | 475 | 18.410 | 74.669 | 12.135 | 1.00 | 23.24 |
| 968 | N | VAL | A | 476 | 20.476 | 75.542 | 12.253 | 1.00 | 24.48 |
| 969 | CA | VAL | A | 476 | 20.038 | 76.908 | 12.446 | 1.00 | 26.25 |
| 970 | CB | VAL | A | 476 | 20.672 | 77.503 | 13.721 | 1.00 | 24.14 |
| 971 | CG1 | VAL | A | 476 | 20.260 | 78.965 | 13.873 | 1.00 | 23.36 |
| 972 | CG2 | VAL | A | 476 | 20.243 | 76.693 | 14.937 | 1.00 | 28.16 |
| 973 | C | VAL | A | 476 | 20.489 | 77.723 | 11.253 | 1.00 | 28.80 |
| 974 | O | VAL | A | 476 | 21.607 | 77.555 | 10.761 | 1.00 | 32.13 |
| 975 | N | GLN | A | 477 | 19.629 | 78.607 | 10.774 | 1.00 | 28.79 |
| 976 | CA | GLN | A | 477 | 20.011 | 79.424 | 9.642 | 1.00 | 30.02 |
| 977 | CB | GLN | A | 477 | 19.746 | 78.687 | 8.326 | 1.00 | 31.30 |
| 978 | CG | GLN | A | 477 | 18.330 | 78.240 | 8.107 | 1.00 | 46.28 |
| 979 | CD | GLN | A | 477 | 18.232 | 77.101 | 7.093 | 1.00 | 49.95 |
| 980 | OE1 | GLN | A | 477 | 17.145 | 76.758 | 6.635 | 1.00 | 51.94 |
| 981 | NE2 | GLN | A | 477 | 19.373 | 76.503 | 6.751 | 1.00 | 51.51 |
| 982 | C | GLN | A | 477 | 19.287 | 80.744 | 9.669 | 1.00 | 30.41 |
| 983 | O | GLN | A | 477 | 18.185 | 80.843 | 10.198 | 1.00 | 30.03 |
| 984 | N | TRP | A | 478 | 19.941 | 81.759 | 9.124 | 1.00 | 29.86 |
| 985 | CA | TRP | A | 478 | 19.390 | 83.095 | 9.058 | 1.00 | 29.12 |
| 986 | CB | TRP | A | 478 | 20.435 | 84.101 | 9.521 | 1.00 | 31.11 |
| 987 | CG | TRP | A | 478 | 20.664 | 84.073 | 10.988 | 1.00 | 36.98 |
| 988 | CD2 | TRP | A | 478 | 19.995 | 84.874 | 11.968 | 1.00 | 34.77 |
| 989 | CE2 | TRP | A | 478 | 20.516 | 84.508 | 13.230 | 1.00 | 37.75 |
| 990 | CE3 | TRP | A | 478 | 18.998 | 85.858 | 11.906 | 1.00 | 31.61 |
| 991 | CD1 | TRP | A | 478 | 21.540 | 83.280 | 11.668 | 1.00 | 35.86 |
| 992 | NE1 | TRP | A | 478 | 21.458 | 83.535 | 13.014 | 1.00 | 35.58 |
| 993 | CZ2 | TRP | A | 478 | 20.079 | 85.106 | 14.432 | 1.00 | 26.96 |
| 994 | CZ3 | TRP | A | 478 | 18.561 | 86.452 | 13.095 | 1.00 | 35.86 |
| 995 | CH2 | TRP | A | 478 | 19.103 | 86.069 | 14.343 | 1.00 | 31.66 |
| 996 | C | TRP | A | 478 | 18.952 | 83.425 | 7.632 | 1.00 | 29.01 |
| 997 | O | TRP | A | 478 | 19.594 | 83.020 | 6.661 | 1.00 | 24.19 |
| 998 | N | LEU | A | 479 | 17.858 | 84.164 | 7.518 | 1.00 | 31.15 |
| 999 | CA | LEU | A | 479 | 17.325 | 84.558 | 6.220 | 1.00 | 36.67 |
| 1000 | CB | LEU | A | 479 | 16.063 | 83.763 | 5.902 | 1.00 | 41.07 |
| 1001 | CG | LEU | A | 479 | 16.016 | 82.272 | 6.233 | 1.00 | 48.71 |
| 1002 | CD1 | LEU | A | 479 | 14.739 | 81.711 | 5.660 | 1.00 | 57.57 |
| 1003 | CD2 | LEU | A | 479 | 17.204 | 81.546 | 5.645 | 1.00 | 56.00 |
| 1004 | C | LEU | A | 479 | 16.976 | 86.044 | 6.180 | 1.00 | 38.99 |
| 1005 | O | LEU | A | 479 | 16.602 | 86.635 | 7.191 | 1.00 | 37.96 |
| 1006 | N | HIS | A | 480 | 17.100 | 86.637 | 5.004 | 1.00 | 42.00 |
| 1007 | CA | HIS | A | 480 | 16.766 | 88.039 | 4.816 | 1.00 | 48.67 |
| 1008 | CB | HIS | A | 480 | 17.952 | 88.816 | 4.229 | 1.00 | 50.22 |
| 1009 | CG | HIS | A | 480 | 17.706 | 90.291 | 4.086 | 1.00 | 53.99 |
| 1010 | CD2 | HIS | A | 480 | 18.559 | 91.344 | 4.107 | 1.00 | 56.80 |
| 1011 | ND1 | HIS | A | 480 | 16.453 | 90.821 | 3.861 | 1.00 | 54.56 |
| 1012 | CE1 | HIS | A | 480 | 16.543 | 92.135 | 3.752 | 1.00 | 60.76 |
| 1013 | NE2 | HIS | A | 480 | 17.811 | 92.478 | 3.897 | 1.00 | 59.92 |
| 1014 | C | HIS | A | 480 | 15.628 | 87.975 | 3.817 | 1.00 | 52.66 |
| 1015 | O | HIS | A | 480 | 15.823 | 88.174 | 2.618 | 1.00 | 51.85 |
| 1016 | N | ASN | A | 481 | 14.439 | 87.655 | 4.320 | 1.00 | 58.88 |
| 1017 | CA | ASN | A | 481 | 13.261 | 87.538 | 3.472 | 1.00 | 63.74 |
| 1018 | CB | ASN | A | 481 | 13.012 | 88.837 | 2.693 | 1.00 | 69.70 |
| 1019 | CG | ASN | A | 481 | 12.389 | 89.926 | 3.539 | 1.00 | 76.04 |
| 1020 | OD1 | ASN | A | 481 | 12.354 | 91.087 | 3.138 | 1.00 | 80.67 |
| 1021 | ND2 | ASN | A | 481 | 11.881 | 89.558 | 4.708 | 1.00 | 82.88 |
| 1022 | C | ASN | A | 481 | 13.466 | 86.418 | 2.467 | 1.00 | 63.93 |
| 1023 | O | ASN | A | 481 | 14.198 | 86.592 | 1.496 | 1.00 | 67.09 |
| 1024 | N | GLU | A | 482 | 12.819 | 85.278 | 2.701 | 1.00 | 62.43 |
| 1025 | CA | GLU | A | 482 | 12.885 | 84.123 | 1.801 | 1.00 | 60.15 |
| 1026 | CB | GLU | A | 482 | 11.977 | 84.375 | 0.581 | 1.00 | 65.85 |
| 1027 | CG | GLU | A | 482 | 11.579 | 85.846 | 0.393 | 1.00 | 78.14 |
| 1028 | CD | GLU | A | 482 | 11.654 | 86.318 | −1.044 | 1.00 | 84.88 |
| 1029 | OE1 | GLU | A | 482 | 12.616 | 85.938 | −1.746 | 1.00 | 90.19 |
| 1030 | OE2 | GLU | A | 482 | 10.759 | 87.082 | −1.466 | 1.00 | 87.19 |
| 1031 | C | GLU | A | 482 | 14.269 | 83.651 | 1.314 | 1.00 | 54.93 |
| 1032 | O | GLU | A | 482 | 14.406 | 82.516 | 0.853 | 1.00 | 57.04 |
| 1033 | N | VAL | A | 483 | 15.295 | 84.489 | 1.414 | 1.00 | 49.03 |
| 1034 | CA | VAL | A | 483 | 16.613 | 84.089 | 0.937 | 1.00 | 45.46 |
| 1035 | CB | VAL | A | 483 | 17.209 | 85.150 | −0.026 | 1.00 | 43.71 |
| 1036 | CG1 | VAL | A | 483 | 17.320 | 86.501 | 0.673 | 1.00 | 50.19 |
| 1037 | CG2 | VAL | A | 483 | 18.567 | 84.687 | −0.533 | 1.00 | 43.76 |
| 1038 | C | VAL | A | 483 | 17.584 | 83.842 | 2.078 | 1.00 | 43.31 |
| 1039 | O | VAL | A | 483 | 17.741 | 84.676 | 2.963 | 1.00 | 41.21 |
| 1040 | N | GLN | A | 484 | 18.238 | 82.687 | 2.039 | 1.00 | 42.10 |
| 1041 | CA | GLN | A | 484 | 19.189 | 82.297 | 3.064 | 1.00 | 41.31 |
| 1042 | CB | GLN | A | 484 | 19.349 | 80.778 | 3.053 | 1.00 | 46.93 |
| 1043 | CG | GLN | A | 484 | 20.420 | 80.262 | 3.993 | 1.00 | 54.66 |
| 1044 | CD | GLN | A | 484 | 20.428 | 78.749 | 4.101 | 1.00 | 61.50 |
| 1045 | OE1 | GLN | A | 484 | 21.402 | 78.158 | 4.568 | 1.00 | 65.59 |
| 1046 | NE2 | GLN | A | 484 | 19.333 | 78.114 | 3.684 | 1.00 | 62.36 |
| 1047 | C | GLN | A | 484 | 20.564 | 82.958 | 2.959 | 1.00 | 40.69 |
| 1048 | O | GLN | A | 484 | 21.124 | 83.097 | 1.868 | 1.00 | 39.56 |
| 1049 | N | LEU | A | 485 | 21.098 | 83.358 | 4.111 | 1.00 | 34.74 |
| 1050 | CA | LEU | A | 485 | 22.408 | 83.998 | 4.191 | 1.00 | 36.73 |
| 1051 | CB | LEU | A | 485 | 22.511 | 84.879 | 5.445 | 1.00 | 36.78 |
| 1052 | CG | LEU | A | 485 | 21.501 | 86.011 | 5.643 | 1.00 | 38.16 |
| 1053 | CD1 | LEU | A | 485 | 21.769 | 86.724 | 6.966 | 1.00 | 39.22 |
| 1054 | CD2 | LEU | A | 485 | 21.607 | 86.969 | 4.479 | 1.00 | 41.68 |
| 1055 | C | LEU | A | 485 | 23.485 | 82.923 | 4.266 | 1.00 | 36.82 |
| 1056 | O | LEU | A | 485 | 23.212 | 81.795 | 4.660 | 1.00 | 34.53 |
| 1057 | N | PRO | A | 486 | 24.725 | 83.258 | 3.883 | 1.00 | 40.02 |
| 1058 | CD | PRO | A | 486 | 25.251 | 84.563 | 3.448 | 1.00 | 39.74 |
| 1059 | CA | PRO | A | 486 | 25.791 | 82.249 | 3.949 | 1.00 | 45.08 |
| 1060 | CB | PRO | A | 486 | 27.018 | 82.994 | 3.422 | 1.00 | 44.97 |
| 1061 | CG | PRO | A | 486 | 26.457 | 84.162 | 2.661 | 1.00 | 41.94 |
| 1062 | C | PRO | A | 486 | 25.971 | 81.862 | 5.413 | 1.00 | 49.91 |
| 1063 | O | PRO | A | 486 | 25.830 | 82.711 | 6.291 | 1.00 | 49.23 |
| 1064 | N | ASP | A | 487 | 26.291 | 80.599 | 5.677 | 1.00 | 54.02 |
| 1065 | CA | ASP | A | 487 | 26.486 | 80.131 | 7.046 | 1.00 | 58.50 |
| 1066 | CB | ASP | A | 487 | 26.851 | 78.647 | 7.039 | 1.00 | 68.77 |
| 1067 | CG | ASP | A | 487 | 26.662 | 77.993 | 8.392 | 1.00 | 79.77 |
| 1068 | OD1 | ASP | A | 487 | 27.164 | 78.545 | 9.397 | 1.00 | 87.08 |
| 1069 | OD2 | ASP | A | 487 | 26.016 | 76.921 | 8.448 | 1.00 | 87.04 |
| 1070 | C | ASP | A | 487 | 27.594 | 80.928 | 7.746 | 1.00 | 57.99 |
| 1071 | O | ASP | A | 487 | 27.445 | 81.352 | 8.898 | 1.00 | 58.25 |
| 1072 | N | ALA | A | 488 | 28.695 | 81.135 | 7.029 | 1.00 | 55.74 |
| 1073 | CA | ALA | A | 488 | 29.852 | 81.861 | 7.546 | 1.00 | 55.27 |
| 1074 | CB | ALA | A | 488 | 30.833 | 82.122 | 6.418 | 1.00 | 55.02 |
| 1075 | C | ALA | A | 488 | 29.511 | 83.177 | 8.239 | 1.00 | 54.82 |
| 1076 | O | ALA | A | 488 | 30.326 | 83.726 | 8.983 | 1.00 | 55.09 |
| 1077 | N | ARG | A | 489 | 28.307 | 83.682 | 8.004 | 1.00 | 52.93 |
| 1078 | CA | ARG | A | 489 | 27.899 | 84.944 | 8.599 | 1.00 | 48.90 |
| 1079 | CB | ARG | A | 489 | 26.799 | 85.571 | 7.749 | 1.00 | 47.45 |
| 1080 | CG | ARG | A | 489 | 27.336 | 86.343 | 6.563 | 1.00 | 49.40 |
| 1081 | CD | ARG | A | 489 | 27.263 | 87.828 | 6.844 | 1.00 | 52.76 |
| 1082 | NE | ARG | A | 489 | 26.103 | 88.426 | 6.190 | 1.00 | 60.57 |
| 1083 | CZ | ARG | A | 489 | 25.513 | 89.559 | 6.560 | 1.00 | 60.40 |
| 1084 | NH1 | ARG | A | 489 | 25.959 | 90.247 | 7.604 | 1.00 | 61.65 |
| 1085 | NH2 | ARG | A | 489 | 24.480 | 90.013 | 5.866 | 1.00 | 62.45 |
| 1086 | C | ARG | A | 489 | 27.456 | 84.894 | 10.058 | 1.00 | 45.96 |
| 1087 | O | ARG | A | 489 | 27.501 | 85.912 | 10.746 | 1.00 | 45.86 |
| 1088 | N | HIS | A | 490 | 27.032 | 83.733 | 10.541 | 1.00 | 41.69 |
| 1089 | CA | HIS | A | 490 | 26.580 | 83.655 | 11.928 | 1.00 | 38.41 |
| 1090 | CB | HIS | A | 490 | 25.087 | 83.286 | 11.997 | 1.00 | 39.34 |
| 1091 | CG | HIS | A | 490 | 24.775 | 81.897 | 11.530 | 1.00 | 38.03 |
| 1092 | CD2 | HIS | A | 490 | 24.784 | 80.715 | 12.189 | 1.00 | 41.49 |
| 1093 | ND1 | HIS | A | 490 | 24.443 | 81.606 | 10.224 | 1.00 | 44.03 |
| 1094 | CE1 | HIS | A | 490 | 24.263 | 80.302 | 10.099 | 1.00 | 42.68 |
| 1095 | NE2 | HIS | A | 490 | 24.465 | 79.740 | 11.277 | 1.00 | 46.75 |
| 1096 | C | HIS | A | 490 | 27.382 | 82.679 | 12.778 | 1.00 | 37.56 |
| 1097 | O | HIS | A | 490 | 28.117 | 81.852 | 12.260 | 1.00 | 38.74 |
| 1098 | N | SER | A | 491 | 27.241 | 82.796 | 14.094 | 1.00 | 36.46 |
| 1099 | CA | SER | A | 491 | 27.926 | 81.906 | 15.021 | 1.00 | 35.63 |
| 1100 | CB | SER | A | 491 | 28.940 | 82.698 | 15.845 | 1.00 | 34.76 |
| 1101 | OG | SER | A | 491 | 29.650 | 81.849 | 16.726 | 1.00 | 51.24 |
| 1102 | C | SER | A | 491 | 26.888 | 81.245 | 15.935 | 1.00 | 32.46 |
| 1103 | O | SER | A | 491 | 26.122 | 81.928 | 16.618 | 1.00 | 34.26 |
| 1104 | N | THR | A | 492 | 26.868 | 79.919 | 15.946 | 1.00 | 30.35 |
| 1105 | CA | THR | A | 492 | 25.922 | 79.147 | 16.750 | 1.00 | 32.31 |
| 1106 | CB | THR | A | 492 | 25.104 | 78.179 | 15.837 | 1.00 | 33.20 |
| 1107 | OG1 | THR | A | 492 | 24.459 | 78.933 | 14.806 | 1.00 | 38.93 |
| 1108 | CG2 | THR | A | 492 | 24.046 | 77.415 | 16.622 | 1.00 | 30.28 |
| 1109 | C | THR | A | 492 | 26.639 | 78.328 | 17.835 | 1.00 | 32.83 |
| 1110 | O | THR | A | 492 | 27.638 | 77.670 | 17.565 | 1.00 | 34.30 |
| 1111 | N | THR | A | 493 | 26.118 | 78.360 | 19.058 | 1.00 | 32.82 |
| 1112 | CA | THR | A | 493 | 26.719 | 77.610 | 20.161 | 1.00 | 32.09 |
| 1113 | CB | THR | A | 493 | 26.254 | 78.151 | 21.530 | 1.00 | 27.96 |
| 1114 | OG1 | THR | A | 493 | 24.825 | 78.059 | 21.630 | 1.00 | 29.42 |
| 1115 | CG2 | THR | A | 493 | 26.680 | 79.600 | 21.694 | 1.00 | 21.13 |
| 1116 | C | THR | A | 493 | 26.336 | 76.131 | 20.067 | 1.00 | 34.06 |
| 1117 | O | THR | A | 493 | 25.436 | 75.770 | 19.317 | 1.00 | 31.49 |
| 1118 | N | CLN | A | 494 | 27.030 | 75.282 | 20.816 | 1.00 | 35.84 |
| 1119 | CA | GLN | A | 494 | 26.740 | 73.848 | 20.824 | 1.00 | 40.51 |
| 1120 | CB | CLN | A | 494 | 27.929 | 73.056 | 21.382 | 1.00 | 46.41 |
| 1121 | CG | GLN | A | 494 | 29.169 | 73.078 | 20.504 | 1.00 | 63.89 |
| 1122 | CD | GLN | A | 494 | 28.890 | 72.592 | 19.093 | 1.00 | 72.04 |
| 1123 | OE1 | CLN | A | 494 | 28.272 | 71.547 | 18.894 | 1.00 | 77.06 |
| 1124 | NE2 | CLN | A | 494 | 29.355 | 73.350 | 18.104 | 1.00 | 81.58 |
| 1125 | C | GLN | A | 494 | 25.512 | 73.596 | 21.691 | 1.00 | 39.51 |
| 1126 | O | CLN | A | 494 | 25.304 | 74.270 | 22.698 | 1.00 | 38.74 |
| 1127 | N | PRO | A | 495 | 24.676 | 72.623 | 21.305 | 1.00 | 38.63 |
| 1128 | CD | PRO | A | 495 | 24.687 | 71.849 | 20.051 | 1.00 | 37.82 |
| 1129 | CA | PRO | A | 495 | 23.480 | 72.330 | 22.100 | 1.00 | 39.10 |
| 1130 | CB | PRO | A | 495 | 22.889 | 71.124 | 21.391 | 1.00 | 37.94 |
| 1131 | CG | PRO | A | 495 | 23.255 | 71.388 | 19.954 | 1.00 | 43.34 |
| 1132 | C | PRO | A | 495 | 23.854 | 72.031 | 23.549 | 1.00 | 41.43 |
| 1133 | O | PRO | A | 495 | 24.838 | 71.346 | 23.808 | 1.00 | 42.40 |
| 1134 | N | ARG | A | 496 | 23.086 | 72.567 | 24.487 | 1.00 | 41.33 |
| 1135 | CA | ARG | A | 496 | 23.343 | 72.343 | 25.906 | 1.00 | 46.41 |
| 1136 | CB | ARG | A | 496 | 23.808 | 73.635 | 26.574 | 1.00 | 44.11 |
| 1137 | CG | ARG | A | 496 | 25.263 | 73.943 | 26.332 | 1.00 | 51.48 |
| 1138 | CD | ARG | A | 496 | 25.633 | 75.331 | 26.813 | 1.00 | 56.73 |
| 1139 | NE | ARG | A | 496 | 27.038 | 75.393 | 27.197 | 1.00 | 63.41 |
| 1140 | CZ | ARG | A | 496 | 27.507 | 74.984 | 28.373 | 1.00 | 59.61 |
| 1141 | NH1 | ARG | A | 496 | 26.683 | 74.489 | 29.286 | 1.00 | 60.71 |
| 1142 | NH2 | ARG | A | 496 | 28.804 | 75.066 | 28.634 | 1.00 | 67.27 |
| 1143 | C | ARG | A | 496 | 22.090 | 71.835 | 26.597 | 1.00 | 49.11 |
| 1144 | O | ARG | A | 496 | 20.989 | 72.303 | 26.318 | 1.00 | 48.52 |
| 1145 | N | LYS | A | 497 | 22.255 | 70.870 | 27.492 | 1.00 | 53.35 |
| 1146 | CA | LYS | A | 497 | 21.116 | 70.322 | 28.208 | 1.00 | 60.05 |
| 1147 | CB | LYS | A | 497 | 21.527 | 69.101 | 29.034 | 1.00 | 65.19 |
| 1148 | CG | LYS | A | 497 | 21.910 | 67.887 | 28.212 | 1.00 | 75.27 |
| 1149 | CD | LYS | A | 497 | 22.255 | 66.705 | 29.103 | 1.00 | 80.74 |
| 1150 | CE | LYS | A | 497 | 22.534 | 65.470 | 28.269 | 1.00 | 86.89 |
| 1151 | NZ | LYS | A | 497 | 22.854 | 64.278 | 29.103 | 1.00 | 91.84 |
| 1152 | C | LYS | A | 497 | 20.546 | 71.376 | 29.131 | 1.00 | 62.05 |
| 1153 | O | LYS | A | 497 | 21.184 | 72.391 | 29.403 | 1.00 | 61.50 |
| 1154 | N | THR | A | 498 | 19.335 | 71.135 | 29.604 | 1.00 | 66.47 |
| 1155 | CA | THR | A | 498 | 18.690 | 72.060 | 30.515 | 1.00 | 71.47 |
| 1156 | CB | THR | A | 498 | 17.417 | 72.654 | 29.893 | 1.00 | 73.54 |
| 1157 | OG1 | THR | A | 498 | 17.118 | 71.981 | 28.666 | 1.00 | 77.08 |
| 1158 | CG2 | THR | A | 498 | 17.619 | 74.120 | 29.609 | 1.00 | 75.50 |
| 1159 | C | THR | A | 498 | 18.339 | 71.319 | 31.794 | 1.00 | 72.65 |
| 1160 | O | THR | A | 498 | 18.850 | 70.223 | 32.037 | 1.00 | 72.18 |
| 1161 | N | ALA | A | 499 | 17.477 | 71.918 | 32.610 | 1.00 | 75.02 |
| 1162 | CA | ALA | A | 499 | 17.062 | 71.303 | 33.867 | 1.00 | 77.01 |
| 1163 | CB | ALA | A | 499 | 16.508 | 72.370 | 34.814 | 1.00 | 76.94 |
| 1164 | C | ALA | A | 499 | 16.009 | 70.224 | 33.607 | 1.00 | 77.96 |
| 1165 | O | ALA | A | 499 | 15.034 | 70.099 | 34.350 | 1.00 | 79.10 |
| 1166 | N | CLY | A | 500 | 16.214 | 69.443 | 32.552 | 1.00 | 78.37 |
| 1167 | CA | CLY | A | 500 | 15.273 | 68.390 | 32.217 | 1.00 | 78.64 |
| 1168 | C | CLY | A | 500 | 14.392 | 68.745 | 31.032 | 1.00 | 77.71 |
| 1169 | O | CLY | A | 500 | 13.741 | 67.878 | 30.447 | 1.00 | 78.53 |
| 1170 | N | SER | A | 501 | 14.390 | 70.021 | 30.661 | 1.00 | 76.72 |
| 1171 | CA | SER | A | 501 | 13.567 | 70.501 | 29.555 | 1.00 | 74.10 |
| 1172 | CB | SER | A | 501 | 13.017 | 71.893 | 29.899 | 1.00 | 79.09 |
| 1173 | OG | SER | A | 501 | 14.053 | 72.787 | 30.276 | 1.00 | 82.51 |
| 1174 | C | SER | A | 501 | 14.235 | 70.545 | 28.176 | 1.00 | 70.47 |
| 1175 | O | SER | A | 501 | 14.188 | 71.575 | 27.499 | 1.00 | 71.67 |
| 1176 | N | GLY | A | 502 | 14.849 | 69.435 | 27.761 | 1.00 | 64.36 |
| 1177 | CA | GLY | A | 502 | 15.484 | 69.369 | 26.447 | 1.00 | 55.25 |
| 1178 | C | GLY | A | 502 | 16.799 | 70.111 | 26.211 | 1.00 | 48.43 |
| 1179 | O | GLY | A | 502 | 17.669 | 70.155 | 27.081 | 1.00 | 47.45 |
| 1180 | N | PHE | A | 503 | 16.952 | 70.678 | 25.015 | 1.00 | 41.96 |
| 1181 | CA | PHE | A | 503 | 18.175 | 71.411 | 24.665 | 1.00 | 34.38 |
| 1182 | CB | PHE | A | 503 | 18.975 | 70.675 | 23.582 | 1.00 | 34.97 |
| 1183 | CG | PHE | A | 503 | 19.432 | 69.304 | 23.979 | 1.00 | 28.11 |
| 1184 | CD1 | PHE | A | 503 | 18.548 | 68.232 | 23.974 | 1.00 | 36.44 |
| 1185 | CD2 | PHE | A | 503 | 20.749 | 69.082 | 24.348 | 1.00 | 30.75 |
| 1186 | CE1 | PHE | A | 503 | 18.971 | 66.960 | 24.338 | 1.00 | 37.43 |
| 1187 | CE2 | PHE | A | 503 | 21.179 | 67.812 | 24.716 | 1.00 | 40.11 |
| 1188 | CZ | PHE | A | 503 | 20.289 | 66.750 | 24.709 | 1.00 | 37.06 |
| 1189 | C | PHE | A | 503 | 17.922 | 72.825 | 24.167 | 1.00 | 28.77 |
| 1190 | O | PHE | A | 503 | 16.816 | 73.176 | 23.756 | 1.00 | 28.23 |
| 1191 | N | PHE | A | 504 | 18.963 | 73.637 | 24.213 | 1.00 | 24.91 |
| 1192 | CA | PHE | A | 504 | 18.887 | 75.006 | 23.743 | 1.00 | 25.79 |
| 1193 | CB | PHE | A | 504 | 18.644 | 76.006 | 24.886 | 1.00 | 22.02 |
| 1194 | CG | PHE | A | 504 | 19.834 | 76.257 | 25.774 | 1.00 | 30.92 |
| 1195 | CD1 | PHE | A | 504 | 20.722 | 77.300 | 25.499 | 1.00 | 32.77 |
| 1196 | CD2 | PHE | A | 504 | 20.037 | 75.487 | 26.919 | 1.00 | 34.76 |
| 1197 | CE1 | PHE | A | 504 | 21.796 | 77.570 | 26.349 | 1.00 | 29.72 |
| 1198 | CE2 | PHE | A | 504 | 21.107 | 75.748 | 27.774 | 1.00 | 33.59 |
| 1199 | CZ | PHE | A | 504 | 21.987 | 76.799 | 27.480 | 1.00 | 34.51 |
| 1200 | C | PHE | A | 504 | 20.175 | 75.328 | 23.027 | 1.00 | 28.90 |
| 1201 | O | PHE | A | 504 | 21.222 | 74.751 | 23.317 | 1.00 | 26.02 |
| 1202 | N | VAL | A | 505 | 20.071 | 76.252 | 22.080 | 1.00 | 25.50 |
| 1203 | CA | VAL | A | 505 | 21.196 | 76.681 | 21.296 | 1.00 | 25.53 |
| 1204 | CB | VAL | A | 505 | 21.193 | 75.891 | 19.955 | 1.00 | 29.70 |
| 1205 | CG1 | VAL | A | 505 | 20.184 | 76.476 | 19.002 | 1.00 | 25.34 |
| 1206 | CG2 | VAL | A | 505 | 22.563 | 75.837 | 19.365 | 1.00 | 35.22 |
| 1207 | C | VAL | A | 505 | 21.015 | 78.196 | 21.092 | 1.00 | 26.24 |
| 1208 | O | VAL | A | 505 | 19.895 | 78.707 | 21.096 | 1.00 | 22.25 |
| 1209 | N | PHE | A | 506 | 22.125 | 78.913 | 20.950 | 1.00 | 28.62 |
| 1210 | CA | PHE | A | 506 | 22.093 | 80.361 | 20.754 | 1.00 | 25.84 |
| 1211 | CB | PHE | A | 506 | 22.769 | 81.061 | 21.935 | 1.00 | 29.19 |
| 1212 | CG | PHE | A | 506 | 22.789 | 82.547 | 21.827 | 1.00 | 35.82 |
| 1213 | CD1 | PHE | A | 506 | 21.630 | 83.286 | 22.030 | 1.00 | 42.42 |
| 1214 | CD2 | PHE | A | 506 | 23.961 | 83.213 | 21.500 | 1.00 | 42.16 |
| 1215 | CE1 | PHE | A | 506 | 21.644 | 84.671 | 21.910 | 1.00 | 44.85 |
| 1216 | CE2 | PHE | A | 506 | 23.982 | 84.591 | 21.376 | 1.00 | 41.56 |
| 1217 | CZ | PHE | A | 506 | 22.825 | 85.322 | 21.580 | 1.00 | 45.90 |
| 1218 | C | PHE | A | 506 | 22.844 | 80.719 | 19.473 | 1.00 | 23.92 |
| 1219 | O | PHE | A | 506 | 23.924 | 80.195 | 19.225 | 1.00 | 27.54 |
| 1220 | N | SER | A | 507 | 22.286 | 81.618 | 18.677 | 1.00 | 25.96 |
| 1221 | CA | SER | A | 507 | 22.925 | 82.041 | 17.432 | 1.00 | 25.75 |
| 1222 | CB | SER | A | 507 | 22.144 | 81.481 | 16.235 | 1.00 | 24.23 |
| 1223 | OG | SER | A | 507 | 22.674 | 81.957 | 15.012 | 1.00 | 33.35 |
| 1224 | C | SER | A | 507 | 23.027 | 83.574 | 17.364 | 1.00 | 25.49 |
| 1225 | O | SER | A | 507 | 22.070 | 84.284 | 17.648 | 1.00 | 25.61 |
| 1226 | N | ARG | A | 508 | 24.203 | 84.063 | 16.994 | 1.00 | 26.99 |
| 1227 | CA | ARG | A | 508 | 24.475 | 85.497 | 16.907 | 1.00 | 25.78 |
| 1228 | CB | AEG | A | 508 | 25.652 | 85.821 | 17.825 | 1.00 | 24.46 |
| 1229 | CG | ARG | A | 508 | 26.177 | 87.245 | 17.753 | 1.00 | 28.19 |
| 1230 | CD | ARG | A | 508 | 27.409 | 87.349 | 18.638 | 1.00 | 27.68 |
| 1231 | NE | ARG | A | 508 | 27.865 | 88.723 | 18.804 | 1.00 | 38.82 |
| 1232 | CZ | ARG | A | 508 | 28.641 | 89.355 | 17.933 | 1.00 | 40.66 |
| 1233 | NE1 | ARG | A | 508 | 29.046 | 88.726 | 16.840 | 1.00 | 39.61 |
| 1234 | NH2 | ARG | A | 508 | 29.007 | 90.613 | 18.151 | 1.00 | 40.23 |
| 1235 | C | ARG | A | 508 | 24.816 | 85.910 | 15.471 | 1.00 | 24.52 |
| 1236 | O | ARG | A | 508 | 25.594 | 85.234 | 14.810 | 1.00 | 24.88 |
| 1237 | N | LEU | A | 509 | 24.246 | 87.013 | 14.992 | 1.00 | 26.27 |
| 1238 | CA | LEU | A | 509 | 24.512 | 87.490 | 13.622 | 1.00 | 31.33 |
| 1239 | CB | LEU | A | 509 | 23.303 | 87.206 | 12.721 | 1.00 | 32.56 |
| 1240 | CG | LEU | A | 509 | 23.311 | 87.774 | 11.292 | 1.00 | 33.39 |
| 1241 | CD1 | LEU | A | 509 | 24.350 | 87.049 | 10.434 | 1.00 | 27.13 |
| 1242 | CD2 | LEU | A | 509 | 21.921 | 87.614 | 10.691 | 1.00 | 27.35 |
| 1243 | C | LEU | A | 509 | 24.830 | 88.988 | 13.550 | 1.00 | 33.20 |
| 1244 | O | LEU | A | 509 | 23.946 | 89.823 | 13.721 | 1.00 | 35.36 |
| 1245 | N | GLU | A | 510 | 26.091 | 89.330 | 13.286 | 1.00 | 33.43 |
| 1246 | CA | GLU | A | 510 | 26.479 | 90.735 | 13.182 | 1.00 | 37.45 |
| 1247 | CB | GLU | A | 510 | 28.002 | 90.880 | 13.305 | 1.00 | 38.70 |
| 1248 | CG | GLU | A | 510 | 28.548 | 90.352 | 14.629 | 1.00 | 53.61 |
| 1249 | CD | GLU | A | 510 | 30.009 | 90.722 | 14.897 | 1.00 | 62.03 |
| 1250 | OE1 | GLU | A | 510 | 30.877 | 90.439 | 14.042 | 1.00 | 65.31 |
| 1251 | OE2 | GLU | A | 510 | 30.288 | 91.290 | 15.976 | 1.00 | 63.62 |
| 1252 | C | GLU | A | 510 | 26.007 | 91.285 | 11.841 | 1.00 | 37.95 |
| 1253 | O | GLU | A | 510 | 26.187 | 90.635 | 10.808 | 1.00 | 35.84 |
| 1254 | N | VAL | A | 511 | 25.391 | 92.466 | 11.845 | 1.00 | 37.36 |
| 1255 | CA | VAL | A | 511 | 24.903 | 93.048 | 10.596 | 1.00 | 39.69 |
| 1256 | CB | VAL | A | 511 | 23.366 | 93.012 | 10.513 | 1.00 | 29.12 |
| 1257 | CG1 | VAL | A | 511 | 22.875 | 91.590 | 10.689 | 1.00 | 28.52 |
| 1258 | CG2 | VAL | A | 511 | 22.764 | 93.933 | 11.554 | 1.00 | 31.67 |
| 1259 | C | VAL | A | 511 | 25.357 | 94.490 | 10.392 | 1.00 | 45.51 |
| 1260 | O | VAL | A | 511 | 25.776 | 95.156 | 11.344 | 1.00 | 46.84 |
| 1261 | N | THR | A | 512 | 25.269 | 94.964 | 9.150 | 1.00 | 47.32 |
| 1262 | CA | THR | A | 512 | 25.673 | 96.326 | 8.813 | 1.00 | 51.20 |
| 1263 | CE | THR | A | 512 | 25.922 | 96.471 | 7.304 | 1.00 | 49.91 |
| 1264 | OG1 | THR | A | 512 | 24.723 | 96.142 | 6.592 | 1.00 | 46.57 |
| 1265 | CG2 | THR | A | 512 | 27.046 | 95.544 | 6.858 | 1.00 | 49.02 |
| 1266 | C | THR | A | 512 | 24.585 | 97.312 | 9.213 | 1.00 | 54.77 |
| 1267 | O | THR | A | 512 | 23.467 | 96.909 | 9.520 | 1.00 | 54.38 |
| 1268 | N | ARG | A | 513 | 24.916 | 98.602 | 9.206 | 1.00 | 56.48 |
| 1269 | CA | ARG | A | 513 | 23.955 | 99.642 | 9.565 | 1.00 | 56.58 |
| 1270 | CB | ARG | A | 513 | 24.609 | 101.026 | 9.503 | 1.00 | 61.58 |
| 1271 | CG | ARG | A | 513 | 24.030 | 102.030 | 10.489 | 1.00 | 67.62 |
| 1272 | CD | ARG | A | 513 | 22.566 | 102.306 | 10.221 | 1.00 | 75.58 |
| 1273 | NE | ARG | A | 513 | 21.857 | 102.737 | 11.422 | 1.00 | 83.06 |
| 1274 | CZ | ARG | A | 513 | 20.572 | 103.081 | 11.448 | 1.00 | 86.37 |
| 1275 | NH1 | ARG | A | 513 | 19.856 | 103.049 | 10.336 | 1.00 | 88.41 |
| 1276 | NH2 | ARG | A | 513 | 19.998 | 103.446 | 12.585 | 1.00 | 87.87 |
| 1277 | C | ARG | A | 513 | 22.783 | 99.603 | 8.600 | 1.00 | 55.15 |
| 1278 | O | ARG | A | 513 | 21.639 | 99.856 | 8.983 | 1.00 | 55.55 |
| 1279 | N | ALA | A | 514 | 23.076 | 99.281 | 7.345 | 1.00 | 55.02 |
| 1280 | CA | ALA | A | 514 | 22.055 | 99.214 | 6.308 | 1.00 | 53.96 |
| 1281 | CB | ALA | A | 514 | 22.715 | 98.972 | 4.961 | 1.00 | 53.56 |
| 1282 | C | ALA | A | 514 | 21.015 | 98.128 | 6.595 | 1.00 | 54.12 |
| 1283 | O | ALA | A | 514 | 19.819 | 98.413 | 6.681 | 1.00 | 54.41 |
| 1284 | N | GLU | A | 515 | 21.464 | 96.884 | 6.736 | 1.00 | 54.63 |
| 1285 | CA | GLU | A | 515 | 20.535 | 95.798 | 7.011 | 1.00 | 54.64 |
| 1286 | CB | GLU | A | 515 | 21.225 | 94.434 | 6.862 | 1.00 | 58.15 |
| 1287 | CG | GLU | A | 515 | 22.668 | 94.392 | 7.331 | 1.00 | 64.48 |
| 1288 | CD | GLU | A | 515 | 23.379 | 93.107 | 6.926 | 1.00 | 65.38 |
| 1289 | OE1 | GLU | A | 515 | 23.183 | 92.643 | 5.784 | 1.00 | 66.84 |
| 1290 | OE2 | GLU | A | 515 | 24.149 | 92.563 | 7.741 | 1.00 | 68.35 |
| 1291 | C | GLU | A | 515 | 19.929 | 95.956 | 8.395 | 1.00 | 54.36 |
| 1292 | O | GLU | A | 515 | 18.897 | 95.360 | 8.694 | 1.00 | 51.06 |
| 1293 | N | TRP | A | 516 | 20.563 | 96.769 | 9.237 | 1.00 | 55.85 |
| 1294 | CA | TRP | A | 516 | 20.043 | 97.015 | 10.576 | 1.00 | 57.10 |
| 1295 | CB | TRP | A | 516 | 21.062 | 97.755 | 11.447 | 1.00 | 58.50 |
| 1296 | CG | TRP | A | 516 | 20.726 | 97.650 | 12.906 | 1.00 | 67.12 |
| 1297 | CD2 | TRP | A | 516 | 20.128 | 98.660 | 13.726 | 1.00 | 68.04 |
| 1298 | CE2 | TRP | A | 516 | 19.915 | 98.091 | 14.999 | 1.00 | 71.07 |
| 1299 | CE3 | TRP | A | 516 | 19.751 | 99.992 | 13.507 | 1.00 | 74.06 |
| 1300 | CD1 | TRP | A | 516 | 20.851 | 96.541 | 13.697 | 1.00 | 67.58 |
| 1301 | NE1 | TRP | A | 516 | 20.364 | 96.797 | 14.954 | 1.00 | 68.75 |
| 1302 | CZ2 | TRP | A | 516 | 19.337 | 98.807 | 16.052 | 1.00 | 75.59 |
| 1303 | CZ3 | TRP | A | 516 | 19.175 | 100.705 | 14.558 | 1.00 | 76.50 |
| 1304 | CH2 | TRP | A | 516 | 18.975 | 100.109 | 15.813 | 1.00 | 76.13 |
| 1305 | C | TRP | A | 516 | 18.772 | 97.854 | 10.452 | 1.00 | 57.68 |
| 1306 | O | TRP | A | 516 | 17.909 | 97.825 | 11.327 | 1.00 | 57.71 |
| 1307 | N | GLU | A | 517 | 18.665 | 98.604 | 9.357 | 1.00 | 58.53 |
| 1308 | CA | GLU | A | 517 | 17.487 | 99.427 | 9.092 | 1.00 | 60.83 |
| 1309 | CE | GLU | A | 517 | 17.758 | 100.381 | 7.921 | 1.00 | 65.68 |
| 1310 | CG | GLU | A | 517 | 18.237 | 101.768 | 8.325 | 1.00 | 77.69 |
| 1311 | CD | GLU | A | 517 | 17.213 | 102.522 | 9.168 | 1.00 | 86.61 |
| 1312 | OE1 | GLU | A | 517 | 16.066 | 102.036 | 9.301 | 1.00 | 92.24 |
| 1313 | OE2 | GLU | A | 517 | 17.550 | 103.605 | 9.696 | 1.00 | 89.43 |
| 1314 | C | GLU | A | 517 | 16.277 | 98.547 | 8.755 | 1.00 | 58.89 |
| 1315 | O | GLU | A | 517 | 15.135 | 98.871 | 9.088 | 1.00 | 56.98 |
| 1316 | N | ALA | A | 518 | 16.537 | 97.430 | 8.084 | 1.00 | 58.33 |
| 1317 | CA | ALA | A | 518 | 15.479 | 96.503 | 7.702 | 1.00 | 54.78 |
| 1318 | CB | ALA | A | 518 | 15.595 | 96.178 | 6.220 | 1.00 | 55.11 |
| 1319 | C | ALA | A | 518 | 15.597 | 95.227 | 8.540 | 1.00 | 53.56 |
| 1320 | O | ALA | A | 518 | 15.375 | 94.120 | 8.045 | 1.00 | 52.54 |
| 1321 | N | LYS | A | 519 | 15.919 | 95.381 | 9.819 | 1.00 | 51.73 |
| 1322 | CA | LYS | A | 519 | 16.092 | 94.217 | 10.671 | 1.00 | 52.62 |
| 1323 | CB | LYS | A | 519 | 16.719 | 94.617 | 12.004 | 1.00 | 52.68 |
| 1324 | CG | LYS | A | 519 | 15.808 | 95.347 | 12.954 | 1.00 | 53.71 |
| 1325 | CD | LYS | A | 519 | 16.631 | 95.904 | 14.103 | 1.00 | 61.43 |
| 1326 | CE | LYS | A | 519 | 15.781 | 96.701 | 15.064 | 1.00 | 65.64 |
| 1327 | NE | LYS | A | 519 | 16.642 | 97.460 | 16.002 | 1.00 | 74.93 |
| 1328 | C | LYS | A | 519 | 14.814 | 93.440 | 10.915 | 1.00 | 52.55 |
| 1329 | O | LYS | A | 519 | 14.857 | 92.287 | 11.339 | 1.00 | 49.84 |
| 1330 | N | ASP | A | 520 | 13.680 | 94.067 | 10.638 | 1.00 | 54.27 |
| 1331 | CA | ASP | A | 520 | 12.381 | 93.424 | 10.820 | 1.00 | 55.54 |
| 1332 | CB | ASP | A | 520 | 11.263 | 94.454 | 10.669 | 1.00 | 65.40 |
| 1333 | CG | ASP | A | 520 | 11.743 | 95.878 | 10.904 | 1.00 | 75.80 |
| 1334 | OD1 | ASP | A | 520 | 12.217 | 96.174 | 12.024 | 1.00 | 83.22 |
| 1335 | OD2 | ASP | A | 520 | 11.652 | 96.700 | 9.964 | 1.00 | 77.87 |
| 1336 | C | ASP | A | 520 | 12.212 | 92.345 | 9.754 | 1.00 | 53.46 |
| 1337 | O | ASP | A | 520 | 11.319 | 91.500 | 9.843 | 1.00 | 51.37 |
| 1338 | N | GLU | A | 521 | 13.079 | 92.383 | 8.747 | 1.00 | 50.72 |
| 1339 | CA | GLU | A | 521 | 13.016 | 91.422 | 7.658 | 1.00 | 50.66 |
| 1340 | CE | GLU | A | 521 | 13.427 | 92.093 | 6.342 | 1.00 | 54.68 |
| 1341 | CO | GLU | A | 521 | 12.408 | 93.090 | 5.799 | 1.00 | 61.75 |
| 1342 | CD | GLU | A | 521 | 12.961 | 93.937 | 4.670 | 1.00 | 68.36 |
| 1343 | OE1 | GLU | A | 521 | 13.463 | 93.368 | 3.678 | 1.00 | 70.14 |
| 1344 | OE2 | GLU | A | 521 | 12.892 | 95.181 | 4.773 | 1.00 | 75.93 |
| 1345 | C | GLU | A | 521 | 13.869 | 90.182 | 7.885 | 1.00 | 46.52 |
| 1346 | O | GLU | A | 521 | 13.743 | 89.208 | 7.148 | 1.00 | 47.21 |
| 1347 | N | PHE | A | 522 | 14.734 | 90.214 | 8.893 | 1.00 | 42.97 |
| 1348 | CA | PHE | A | 522 | 15.589 | 89.069 | 9.181 | 1.00 | 38.46 |
| 1349 | CB | PHE | A | 522 | 16.848 | 89.509 | 9.915 | 1.00 | 37.28 |
| 1350 | CG | PHE | A | 522 | 17.846 | 90.180 | 9.024 | 1.00 | 42.52 |
| 1351 | CD1 | PHE | A | 522 | 17.675 | 91.510 | 8.646 | 1.00 | 44.12 |
| 1352 | CD2 | PHE | A | 522 | 18.929 | 89.467 | 8.508 | 1.00 | 40.14 |
| 1353 | CE1 | PHE | A | 522 | 18.564 | 92.126 | 7.762 | 1.00 | 48.02 |
| 1354 | CE2 | PHE | A | 522 | 19.825 | 90.072 | 7.623 | 1.00 | 42.77 |
| 1355 | CZ | PHE | A | 522 | 19.640 | 91.408 | 7.248 | 1.00 | 41.32 |
| 1356 | C | PHE | A | 522 | 14.852 | 88.006 | 9.970 | 1.00 | 36.02 |
| 1357 | O | PHE | A | 522 | 14.126 | 88.308 | 10.918 | 1.00 | 37.87 |
| 1358 | N | ILE | A | 523 | 15.042 | 86.758 | 9.561 | 1.00 | 32.13 |
| 1359 | CA | ILE | A | 523 | 14.376 | 85.626 | 10.187 | 1.00 | 31.74 |
| 1360 | CB | ILE | A | 523 | 13.392 | 84.966 | 9.192 | 1.00 | 36.58 |
| 1361 | CG2 | ILE | A | 523 | 12.711 | 83.751 | 9.838 | 1.00 | 38.36 |
| 1362 | CG1 | ILE | A | 523 | 12.367 | 86.000 | 8.714 | 1.00 | 39.38 |
| 1363 | CD1 | ILE | A | 523 | 11.517 | 85.514 | 7.564 | 1.00 | 40.35 |
| 1364 | C | ILE | A | 523 | 15.356 | 84.559 | 10.670 | 1.00 | 30.51 |
| 1365 | O | ILE | A | 523 | 16.369 | 84.294 | 10.034 | 1.00 | 29.34 |
| 1366 | N | CYS | A | 524 | 15.046 | 83.949 | 11.802 | 1.00 | 28.73 |
| 1367 | CA | CYS | A | 524 | 15.879 | 82.898 | 12.351 | 1.00 | 27.60 |
| 1368 | C | CYS | A | 524 | 15.081 | 81.635 | 12.136 | 1.00 | 27.54 |
| 1369 | O | CYS | A | 524 | 13.933 | 81.567 | 12.555 | 1.00 | 28.72 |
| 1370 | CB | CYS | A | 524 | 16.096 | 83.123 | 13.849 | 1.00 | 26.75 |
| 1371 | SG | CYS | A | 524 | 16.999 | 81.777 | 14.689 | 1.00 | 36.31 |
| 1372 | N | ARG | A | 525 | 15.675 | 80.636 | 11.497 | 1.00 | 28.42 |
| 1373 | CA | ARG | A | 525 | 14.966 | 79.400 | 11.226 | 1.00 | 28.35 |
| 1374 | CB | ARG | A | 525 | 14.780 | 79.229 | 9.720 | 1.00 | 34.16 |
| 1375 | CG | ARG | A | 525 | 14.120 | 77.912 | 9.331 | 1.00 | 39.75 |
| 1376 | CD | ARG | A | 525 | 13.508 | 77.996 | 7.937 | 1.00 | 48.43 |
| 1377 | NE | ARG | A | 525 | 14.483 | 77.825 | 6.874 | 1.00 | 52.97 |
| 1378 | CZ | ARG | A | 525 | 14.242 | 78.108 | 5.598 | 1.00 | 58.72 |
| 1379 | NH1 | ARG | A | 525 | 13.054 | 78.580 | 5.233 | 1.00 | 55.56 |
| 1380 | NH2 | ARG | A | 525 | 15.190 | 77.930 | 4.686 | 1.00 | 55.90 |
| 1381 | C | ARG | A | 525 | 15.673 | 78.184 | 11.783 | 1.00 | 28.14 |
| 1382 | O | ARG | A | 525 | 16.889 | 78.055 | 11.691 | 1.00 | 28.95 |
| 1383 | N | ALA | A | 526 | 14.892 | 77.283 | 12.357 | 1.00 | 25.82 |
| 1384 | CA | ALA | A | 526 | 15.440 | 76.082 | 12.930 | 1.00 | 25.90 |
| 1385 | CB | ALA | A | 526 | 15.056 | 75.986 | 14.409 | 1.00 | 28.82 |
| 1386 | C | ALA | A | 526 | 14.871 | 74.903 | 12.169 | 1.00 | 27.38 |
| 1387 | O | ALA | A | 526 | 13.690 | 74.899 | 11.847 | 1.00 | 24.32 |
| 1388 | N | VAL | A | 527 | 15.718 | 73.924 | 11.855 | 1.00 | 24.37 |
| 1389 | CA | VAL | A | 527 | 15.266 | 72.727 | 11.177 | 1.00 | 24.12 |
| 1390 | CB | VAL | A | 527 | 16.054 | 72.434 | 9.899 | 1.00 | 24.30 |
| 1391 | CG1 | VAL | A | 527 | 15.637 | 71.090 | 9.338 | 1.00 | 26.10 |
| 1392 | CG2 | VAL | A | 527 | 15.796 | 73.517 | 8.876 | 1.00 | 30.97 |
| 1393 | C | VAL | A | 527 | 15.523 | 71.646 | 12.198 | 1.00 | 26.78 |
| 1394 | O | VAL | A | 527 | 16.631 | 71.522 | 12.725 | 1.00 | 25.10 |
| 1395 | N | HIS | A | 528 | 14.497 | 70.857 | 12.470 | 1.00 | 26.18 |
| 1396 | CA | HIS | A | 528 | 14.585 | 69.829 | 13.484 | 1.00 | 29.17 |
| 1397 | CB | HIS | A | 528 | 14.413 | 70.490 | 14.879 | 1.00 | 28.69 |
| 1398 | CC | HIS | A | 528 | 14.409 | 69.522 | 16.028 | 1.00 | 30.83 |
| 1399 | CD2 | HIS | A | 528 | 13.456 | 68.661 | 16.461 | 1.00 | 30.29 |
| 1400 | ND1 | HIS | A | 528 | 15.504 | 69.318 | 16.842 | 1.00 | 30.55 |
| 1401 | CE1 | HIS | A | 528 | 15.228 | 68.367 | 17.719 | 1.00 | 28.76 |
| 1402 | NE2 | HIS | A | 528 | 13.992 | 67.950 | 17.507 | 1.00 | 23.82 |
| 1403 | C | HIS | A | 528 | 13.498 | 68.782 | 13.230 | 1.00 | 31.62 |
| 1404 | O | HIS | A | 528 | 12.387 | 69.096 | 12.788 | 1.00 | 30.62 |
| 1405 | N | GLU | A | 529 | 13.834 | 67.539 | 13.543 | 1.00 | 34.89 |
| 1406 | CA | GLU | A | 529 | 12.949 | 66.400 | 13.347 | 1.00 | 41.27 |
| 1407 | CB | GLU | A | 529 | 13.640 | 65.129 | 13.843 | 1.00 | 46.90 |
| 1408 | CG | GLU | A | 529 | 12.721 | 63.908 | 13.872 | 1.00 | 61.52 |
| 1409 | CD | GLU | A | 529 | 13.184 | 62.836 | 14.852 | 1.00 | 68.52 |
| 1410 | OE1 | GLU | A | 529 | 12.433 | 61.860 | 15.064 | 1.00 | 68.05 |
| 1411 | OE2 | GLU | A | 529 | 14.294 | 62.969 | 15.413 | 1.00 | 73.22 |
| 1412 | C | GLU | A | 529 | 11.565 | 66.476 | 13.996 | 1.00 | 42.79 |
| 1413 | O | GLU | A | 529 | 10.578 | 66.069 | 13.381 | 1.00 | 42.08 |
| 1414 | N | ALA | A | 530 | 11.502 | 66.978 | 15.232 | 1.00 | 43.28 |
| 1415 | CA | ALA | A | 530 | 10.250 | 67.061 | 15.987 | 1.00 | 44.89 |
| 1416 | CB | ALA | A | 530 | 10.546 | 67.183 | 17.472 | 1.00 | 42.67 |
| 1417 | C | ALA | A | 530 | 9.263 | 68.142 | 15.582 | 1.00 | 47.12 |
| 1418 | O | ALA | A | 530 | 8.126 | 68.137 | 16.048 | 1.00 | 47.05 |
| 1419 | N | ALA | A | 531 | 9.684 | 69.071 | 14.732 | 1.00 | 50.02 |
| 1420 | CA | ALA | A | 531 | 8.792 | 70.130 | 14.280 | 1.00 | 54.71 |
| 1421 | CB | ALA | A | 531 | 9.602 | 71.233 | 13.621 | 1.00 | 52.38 |
| 1422 | C | ALA | A | 531 | 7.787 | 69.519 | 13.292 | 1.00 | 59.71 |
| 1423 | O | ALA | A | 531 | 7.784 | 69.835 | 12.102 | 1.00 | 57.91 |
| 1424 | N | SER | A | 532 | 6.936 | 68.642 | 13.825 | 1.00 | 66.39 |
| 1425 | CA | SER | A | 532 | 5.911 | 67.899 | 13.078 | 1.00 | 71.61 |
| 1426 | CB | SER | A | 532 | 4.570 | 67.949 | 13.828 | 1.00 | 75.15 |
| 1427 | OG | SER | A | 532 | 4.190 | 69.274 | 14.168 | 1.00 | 81.74 |
| 1428 | C | SER | A | 532 | 5.681 | 68.198 | 11.595 | 1.00 | 73.08 |
| 1429 | O | SER | A | 532 | 5.855 | 67.307 | 10.757 | 1.00 | 75.98 |
| 1430 | N | PRO | A | 533 | 5.279 | 69.433 | 11.236 | 1.00 | 72.21 |
| 1431 | CD | PRO | A | 533 | 4.769 | 70.589 | 11.998 | 1.00 | 72.93 |
| 1432 | CA | PRO | A | 533 | 5.076 | 69.644 | 9.801 | 1.00 | 69.88 |
| 1433 | CB | PRO | A | 533 | 3.913 | 70.617 | 9.775 | 1.00 | 69.60 |
| 1434 | CG | PRO | A | 533 | 4.278 | 71.537 | 10.895 | 1.00 | 71.96 |
| 1435 | C | PRO | A | 533 | 6.317 | 70.212 | 9.113 | 1.00 | 67.12 |
| 1436 | O | PRO | A | 533 | 6.820 | 71.269 | 9.500 | 1.00 | 67.77 |
| 1437 | N | SER | A | 534 | 6.802 | 69.499 | 8.103 | 1.00 | 62.96 |
| 1438 | CA | SER | A | 534 | 7.967 | 69.917 | 7.331 | 1.00 | 56.20 |
| 1439 | CB | SER | A | 534 | 7.609 | 71.137 | 6.479 | 1.00 | 57.74 |
| 1440 | OG | SER | A | 534 | 6.985 | 72.137 | 7.262 | 1.00 | 67.85 |
| 1441 | C | SER | A | 534 | 9.258 | 70.194 | 8.111 | 1.00 | 49.03 |
| 1442 | O | SER | A | 534 | 10.176 | 70.811 | 7.576 | 1.00 | 47.21 |
| 1443 | N | OLN | A | 535 | 9.322 | 69.742 | 9.363 | 1.00 | 42.33 |
| 1444 | CA | OLN | A | 535 | 10.504 | 69.896 | 10.204 | 1.00 | 38.93 |
| 1445 | CB | OLN | A | 535 | 11.588 | 68.887 | 9.784 | 1.00 | 32.30 |
| 1446 | CG | OLN | A | 535 | 11.212 | 67.419 | 9.972 | 1.00 | 35.25 |
| 1447 | CD | GLN | A | 535 | 10.074 | 66.981 | 9.076 | 1.00 | 41.01 |
| 1448 | OE1 | OLN | A | 535 | 10.112 | 67.172 | 7.861 | 1.00 | 43.29 |
| 1449 | NE2 | GLN | A | 535 | 9.055 | 66.380 | 9.669 | 1.00 | 47.79 |
| 1450 | C | OLN | A | 535 | 11.115 | 71.290 | 10.209 | 1.00 | 37.85 |
| 1451 | O | OLN | A | 535 | 12.335 | 71.432 | 10.281 | 1.00 | 36.76 |
| 1452 | N | THR | A | 536 | 10.280 | 72.317 | 10.147 | 1.00 | 37.28 |
| 1453 | CA | THR | A | 536 | 10.795 | 73.675 | 10.145 | 1.00 | 39.00 |
| 1454 | CB | THR | A | 536 | 10.752 | 74.281 | 8.734 | 1.00 | 41.53 |
| 1455 | OG1 | THR | A | 536 | 11.256 | 73.333 | 7.786 | 1.00 | 46.75 |
| 1456 | CG2 | THR | A | 536 | 11.613 | 75.524 | 8.676 | 1.00 | 39.44 |
| 1457 | C | THR | A | 536 | 10.014 | 74.584 | 11.072 | 1.00 | 38.42 |
| 1458 | O | THR | A | 536 | 8.806 | 74.452 | 11.205 | 1.00 | 43.26 |
| 1459 | N | VAL | A | 537 | 10.723 | 75.495 | 11.726 | 1.00 | 37.42 |
| 1460 | CA | VAL | A | 537 | 10.138 | 76.469 | 12.632 | 1.00 | 32.52 |
| 1461 | CB | VAL | A | 537 | 10.134 | 75.981 | 14.110 | 1.00 | 36.29 |
| 1462 | CG1 | VAL | A | 537 | 9.452 | 77.020 | 14.986 | 1.00 | 35.71 |
| 1463 | C02 | VAL | A | 537 | 9.395 | 74.669 | 14.241 | 1.00 | 43.42 |
| 1464 | C | VAL | A | 537 | 11.006 | 77.724 | 12.538 | 1.00 | 31.41 |
| 1465 | O | VAL | A | 537 | 12.231 | 77.654 | 12.665 | 1.00 | 31.67 |
| 1466 | N | OLN | A | 538 | 10.381 | 78.874 | 12.313 | 1.00 | 27.91 |
| 1467 | CA | GLN | A | 538 | 11.148 | 80.104 | 12.183 | 1.00 | 29.41 |
| 1468 | CB | GLN | A | 538 | 11.464 | 80.369 | 10.708 | 1.00 | 29.17 |
| 1469 | CG | GLN | A | 538 | 10.237 | 80.635 | 9.849 | 1.00 | 29.81 |
| 1470 | CD | GLN | A | 538 | 10.617 | 80.890 | 8.409 | 1.00 | 29.58 |
| 1471 | OE1 | GLN | A | 538 | 11.424 | 80.157 | 7.833 | 1.00 | 37.40 |
| 1472 | NE2 | GLN | A | 538 | 10.041 | 81.924 | 7.815 | 1.00 | 32.13 |
| 1473 | C | GLN | A | 538 | 10.407 | 81.296 | 12.744 | 1.00 | 31.40 |
| 1474 | O | GLN | A | 538 | 9.189 | 81.286 | 12.844 | 1.00 | 33.62 |
| 1475 | N | ARG | A | 539 | 11.151 | 82.333 | 13.098 | 1.00 | 31.64 |
| 1476 | CA | ARG | A | 539 | 10.537 | 83.534 | 13.619 | 1.00 | 34.86 |
| 1477 | CB | ARG | A | 539 | 10.480 | 83.480 | 15.145 | 1.00 | 41.64 |
| 1478 | CG | ARG | A | 539 | 9.522 | 84.477 | 15.762 | 1.00 | 48.95 |
| 1479 | CD | ARG | A | 539 | 8.755 | 83.804 | 16.884 | 1.00 | 59.63 |
| 1480 | NE | ARG | A | 539 | 8.205 | 82.533 | 16.420 | 1.00 | 74.02 |
| 1481 | CZ | ARG | A | 539 | 7.506 | 81.691 | 17.173 | 1.00 | 82.05 |
| 1482 | NH1 | ARG | A | 539 | 7.259 | 81.977 | 18.447 | 1.00 | 87.03 |
| 1483 | NH2 | ARG | A | 539 | 7.053 | 80.560 | 16.646 | 1.00 | 85.40 |
| 1484 | C | ARG | A | 539 | 11.311 | 84.760 | 13.178 | 1.00 | 34.74 |
| 1485 | O | ARG | A | 539 | 12.541 | 84.767 | 13.190 | 1.00 | 37.29 |
| 1486 | N | ALA | A | 540 | 10.580 | 85.794 | 12.772 | 1.00 | 34.85 |
| 1487 | CA | ALA | A | 540 | 11.192 | 87.039 | 12.344 | 1.00 | 34.24 |
| 1488 | CB | ALA | A | 540 | 10.175 | 87.891 | 11.628 | 1.00 | 35.02 |
| 1489 | C | ALA | A | 540 | 11.700 | 87.754 | 13.587 | 1.00 | 35.51 |
| 1490 | O | ALA | A | 540 | 11.205 | 87.518 | 14.695 | 1.00 | 30.77 |
| 1491 | N | VAL | A | 541 | 12.704 | 88.604 | 13.408 | 1.00 | 36.19 |
| 1492 | CA | VAL | A | 541 | 13.274 | 89.349 | 14.520 | 1.00 | 42.85 |
| 1493 | CB | VAL | A | 541 | 14.428 | 90.265 | 14.038 | 1.00 | 43.71 |
| 1494 | CG1 | VAL | A | 541 | 14.744 | 91.316 | 15.084 | 1.00 | 48.75 |
| 1495 | CG2 | VAL | A | 541 | 15.665 | 89.433 | 13.749 | 1.00 | 43.59 |
| 1496 | C | VAL | A | 541 | 12.181 | 90.194 | 15.178 | 1.00 | 48.00 |
| 1497 | O | VAL | A | 541 | 11.587 | 91.063 | 14.536 | 1.00 | 44.96 |
| 1498 | N | SER | A | 542 | 11.906 | 89.927 | 16.451 | 1.00 | 53.41 |
| 1499 | CA | SER | A | 542 | 10.883 | 90.686 | 17.152 | 1.00 | 61.94 |
| 1500 | CB | SER | A | 542 | 10.532 | 90.036 | 18.494 | 1.00 | 62.96 |
| 1501 | OG | SER | A | 542 | 11.596 | 90.138 | 19.421 | 1.00 | 69.91 |
| 1502 | C | SER | A | 542 | 11.437 | 92.083 | 17.372 | 1.00 | 65.98 |
| 1503 | O | SER | A | 542 | 12.628 | 92.253 | 17.634 | 1.00 | 67.12 |
| 1504 | N | VAL | A | 543 | 10.571 | 93.080 | 17.249 | 1.00 | 70.21 |
| 1505 | CA | VAL | A | 543 | 10.975 | 94.466 | 17.421 | 1.00 | 74.63 |
| 1506 | CB | VAL | A | 543 | 11.300 | 95.121 | 16.058 | 1.00 | 78.23 |
| 1507 | CG1 | VAL | A | 543 | 11.713 | 96.574 | 16.260 | 1.00 | 79.65 |
| 1508 | CC2 | VAL | A | 543 | 12.409 | 94.344 | 15.359 | 1.00 | 79.80 |
| 1509 | C | VAL | A | 543 | 9.871 | 95.259 | 18.111 | 1.00 | 75.67 |
| 1510 | O | VAL | A | 543 | 8.974 | 95.753 | 17.396 | 1.00 | 77.58 |
| 1511 | OXT | VAL | A | 543 | 9.907 | 95.360 | 19.356 | 1.00 | 74.95 |
| 1512 | C1 | NAG | A | 2 | 39.866 | 71.246 | 15.298 | 1.00 | 97.60 |
| 1513 | O1 | NAG | A | 2 | 39.913 | 72.355 | 16.125 | 1.00 | 99.81 |
| 1514 | C2 | NAG | A | 2 | 38.452 | 71.095 | 14.687 | 1.00 | 96.75 |
| 1515 | N2 | NAG | A | 2 | 38.075 | 72.305 | 13.989 | 1.00 | 98.55 |
| 1516 | C7 | NAG | A | 2 | 38.040 | 72.336 | 12.660 | 1.00 | 101.24 |
| 1517 | O7 | NAG | A | 2 | 38.381 | 71.38S | 11.956 | 1.00 | 100.19 |
| 1518 | C8 | NAG | A | 2 | 37.567 | 73.623 | 12.006 | 1.00 | 100.76 |
| 1519 | C3 | NAG | A | 2 | 37.394 | 70.819 | 1S.772 | 1.00 | 96.21 |
| 1520 | O3 | NAG | A | 2 | 36.138 | 70.S2S | 1S.162 | 1.00 | 95.30 |
| 1521 | C4 | NAG | A | 2 | 37.840 | 69.634 | 16.642 | 1.00 | 94.75 |
| 1522 | O4 | NAG | A | 2 | 36.924 | 69.444 | 17.72S | 1.00 | 91.92 |
| 1523 | C5 | NAG | A | 2 | 39.273 | 69.896 | 17.179 | 1.00 | 95.52 |
| 1524 | O5 | NAG | A | 2 | 40.204 | 70.093 | 16.083 | 1.00 | 96.34 |
| 1525 | C6 | NAG | A | 2 | 39.775 | 68.727 | 17.999 | 1.00 | 96.13 |
| 1526 | O6 | NAG | A | 2 | 39.723 | 67.507 | 17.266 | 1.00 | 99.34 |
| 1527 | OH2 | TIP | A | 1 | 24.621 | 77.330 | 12.183 | 1.00 | 50.26 |
| 1528 | OH2 | TIP | A | 2 | 17.289 | 54.802 | 2.062 | 1.00 | 49.71 |
| 1529 | OH2 | TIP | A | 3 | 10.654 | 89.058 | −3.198 | 1.00 | 62.73 |
| 1530 | OH2 | TIP | A | 4 | 22.344 | 80.875 | 7.156 | 1.00 | 44.66 |
| 1531 | OE2 | TIP | A | 5 | 27.537 | 65.331 | 9.249 | 1.00 | 53.56 |
| 1532 | OH2 | TIP | A | 6 | 26.192 | 96.403 | 20.749 | 1.00 | 44.98 |
| 1533 | OH2 | TIP | A | 7 | 8.855 | 79.202 | 17.894 | 1.00 | 44.08 |
| 1534 | OH2 | TIP | A | 8 | 16.591 | 66.982 | 14.521 | 1.00 | 40.39 |
| 1535 | OH2 | TIP | A | 9 | 24.434 | 65.928 | 6.674 | 1.00 | 40.49 |
| 1536 | OH2 | TIP | A | 10 | 27.385 | 88.484 | 9.939 | 1.00 | 46.89 |
| 1537 | OH2 | TIP | A | 11 | 28.304 | 87.357 | 13.183 | 1.00 | 50.51 |
| 1538 | OH2 | TIP | A | 12 | 28.907 | 78.522 | 14.088 | 1.00 | 50.17 |
| 1539 | OH2 | TIP | A | 13 | 47.570 | 70.510 | 13.386 | 1.00 | 54.70 |
| 1540 | OH2 | TIP | A | 14 | 23.814 | 76.382 | 23.745 | 1.00 | 44.57 |
| 1541 | OH2 | TIP | A | 15 | 22.971 | 61.676 | 0.664 | 1.00 | 50.94 |
| 1542 | OH2 | TIP | A | 16 | 19.616 | 104.062 | 18.276 | 1.00 | 56.21 |
| 1543 | OH2 | TIP | A | 17 | 30.294 | 75.010 | 30.820 | 1.00 | 59.52 |
| 1544 | OH2 | TIP | A | 18 | 10.090 | 82.420 | 4.828 | 1.00 | 55.58 |
| 1545 | OH2 | TIP | A | 19 | 45.243 | 73.089 | 9.124 | 1.00 | 66.56 |
| 1546 | OH2 | TIP | A | 20 | 46.743 | 70.603 | 11.012 | 1.00 | 63.77 |
| 1547 | OH2 | TIP | A | 21 | 29.058 | 92.561 | 8.613 | 1.00 | 62.34 |
| 1548 | OH2 | TIP | A | 22 | 26.556 | 59.226 | 16.825 | 1.00 | 54.44 |
| 1549 | OH2 | TIP | A | 23 | 26.041 | 54.669 | 2.219 | 1.00 | 52.42 |
| 1550 | OH2 | TIP | A | 24 | 34.966 | 68.214 | 16.774 | 1.00 | 60.14 |
| 1551 | OH2 | TIP | A | 25 | 20.048 | 55.636 | 12.052 | 1.00 | 48.11 |
| 1552 | OH2 | TIP | A | 26 | 28.830 | 86.081 | 15.665 | 1.00 | 45.10 |
| 1553 | OH2 | TIP | A | 27 | 34.158 | 69.085 | 18.975 | 1.00 | 59.32 |
| 1554 | OH2 | TIP | A | 28 | 28.875 | 47.094 | 5.425 | 1.00 | 53.61 |
| 1555 | OH2 | TIP | A | 29 | 26.558 | 67.506 | 3.036 | 1.00 | 60.13 |
| 1556 | OH2 | TIP | A | 30 | 38.399 | 52.730 | 18.974 | 1.00 | 56.34 |
| 1557 | OH2 | TIP | A | 31 | 23.500 | 72.144 | 10.139 | 1.00 | 58.14 |
| 1558 | OH2 | TIP | A | 32 | 34.771 | 64.807 | 3.542 | 1.00 | 50.57 |
| 1559 | OH2 | TIP | A | 33 | 24.268 | 95.724 | 3.944 | 1.00 | 56.48 |
| 1560 | OH2 | TIP | A | 34 | 27.690 | 78.824 | 11.826 | 1.00 | 60.64 |
| 1561 | OH2 | TIP | A | 35 | 49.321 | 73.518 | 9.173 | 1.00 | 56.75 |
| 1562 | OH2 | TIP | A | 36 | 20.391 | 63.685 | 13.114 | 1.00 | 52.49 |
| 1563 | OH2 | TIP | A | 37 | 25.216 | 70.518 | 28.590 | 1.00 | 56.17 |
| 1564 | OH2 | TIP | A | 38 | 42.408 | 48.356 | 24.205 | 1.00 | 57.49 |
| 1565 | OH2 | TIP | A | 39 | 29.180 | 99.267 | 16.299 | 1.00 | 59.27 |
| 1566 | OH2 | TIP | A | 40 | 14.998 | 75.915 | 2.509 | 1.00 | 55.17 |
| 1567 | OH2 | TIP | A | 41 | 25.941 | 69.212 | 22.056 | 1.00 | 55.25 |
| 1568 | OH2 | TIP | A | 42 | 11.307 | 86.566 | 17.528 | 1.00 | 50.93 |
| 1569 | OH2 | TIP | A | 43 | 26.577 | 64.924 | 15.413 | 1.00 | 58.05 |
| 1570 | OH2 | TIP | A | 44 | 9.305 | 75.672 | 24.091 | 1.00 | 63.22 |
| 1571 | OH2 | TIP | A | 45 | 31.263 | 91.102 | 9.512 | 1.00 | 59.76 |
| 1572 | OH2 | TIP | A | 46 | 29.116 | 71.553 | 15.523 | 1.00 | 61.09 |
| 1573 | OH2 | TIP | A | 47 | 20.286 | 50.431 | 1.295 | 1.00 | 58.75 |
| 1574 | OH2 | TIP | A | 48 | 39.170 | 57.553 | 3.300 | 1.00 | 59.73 |
| 1575 | OH2 | TIP | A | 49 | 8.842 | 71.660 | 23.296 | 1.00 | 53.77 |
| 1576 | OH2 | TIP | A | 50 | 12.559 | 48.527 | 9.562 | 1.00 | 59.31 |
| 1577 | OH2 | TIP | A | 51 | 28.206 | 76.115 | 14.977 | 1.00 | 56.85 |
| 1578 | OH2 | TIP | A | 52 | 8.825 | 77.060 | 27.183 | 1.00 | 60.05 |
| 1579 | OH2 | TIP | A | 53 | 32.649 | 60.706 | 17.690 | 1.00 | 57.10 |
| 1580 | OH2 | TIP | A | 54 | 49.662 | 60.238 | 13.997 | 1.00 | 60.54 |
| 1581 | OH2 | TIP | A | 55 | 30.363 | 57.461 | 17.811 | 1.00 | 62.47 |
| 1582 | OH2 | TIP | A | 56 | 24.541 | 106.634 | 19.860 | 1.00 | 64.33 |
| 1583 | OH2 | TIP | A | 57 | 11.412 | 70.168 | 26.131 | 1.00 | 52.42 |
| 1584 | OH2 | TIP | A | 58 | 10.025 | 88.314 | 3.596 | 1.00 | 60.20 |
| 1585 | OH2 | TIP | A | 59 | 22.043 | 63.628 | 26.199 | 1.00 | 59.26 |
| 1586 | OH2 | TIP | A | 60 | 46.414 | 76.131 | 6.439 | 1.00 | 62.09 |
| 1587 | OH2 | TIP | A | 61 | 22.767 | 54.070 | 3.523 | 1.00 | 65.16 |
| 1588 | OH2 | TIP | A | 62 | 14.610 | 78.959 | 1.837 | 1.00 | 59.85 |
| 1589 | OH2 | TIP | A | 63 | 18.102 | 59.201 | 0.758 | 1.00 | 56.88 |
| 1590 | OH2 | TIP | A | 64 | 29.616 | 80.265 | 4.265 | 1.00 | 53.06 |
| 1591 | OH2 | TIP | A | 65 | 18.383 | 97.407 | 19.064 | 1.00 | 60.26 |
| 1592 | OH2 | TIP | A | 66 | 16.855 | 77.568 | 2.254 | 1.00 | 59.03 |
| 1593 | OH2 | TIP | A | 67 | 32.757 | 64.887 | 12.306 | 1.00 | 54.95 |
| 1594 | OH2 | TIP | A | 68 | 27.226 | 97.417 | 12.532 | 1.00 | 57.65 |
| 1595 | OH2 | TIP | A | 69 | 16.812 | 67.479 | 30.053 | 1.00 | 59.41 |
| 1596 | OH2 | TIP | A | 70 | 12.053 | 64.929 | 3.775 | 1.00 | 62.27 |
| 1597 | OH2 | TIP | A | 71 | 36.626 | 74.187 | 14.978 | 1.00 | 55.86 |
| 1598 | OH2 | TIP | A | 72 | 33.116 | 54.343 | 0.777 | 1.00 | 59.39 |
| 1599 | OH2 | TIP | A | 73 | 21.701 | 75.116 | 6.786 | 1.00 | 59.25 |
| 1600 | OH2 | TIP | A | 74 | 33.920 | 50.039 | 9.248 | 1.00 | 53.17 |
| 1601 | OH2 | TIP | A | 75 | 10.687 | 52.377 | 4.811 | 1.00 | 65.55 |
| 1602 | OH2 | TIP | A | 76 | 41.791 | 50.133 | 21.910 | 1.00 | 62.05 |
| 1603 | OH2 | TIP | A | 77 | 38.603 | 74.281 | 16.682 | 1.00 | 57.87 |
| 1604 | OH2 | TIP | A | 78 | 11.460 | 76.303 | 28.686 | 1.00 | 58.31 |
| 1605 | OH2 | TIP | A | 79 | 22.506 | 95.718 | 17.568 | 1.00 | 53.97 |
| 1606 | OH2 | TIP | A | 80 | 20.912 | 49.812 | 13.914 | 1.00 | 54.45 |
| 1607 | OH2 | TIP | A | 81 | 21.594 | 91.198 | 3.704 | 1.00 | 60.50 |
| 1608 | OH2 | TIP | A | 82 | 5.956 | 84.199 | 15.573 | 1.00 | 54.25 |
| 1609 | OH2 | TIP | A | 83 | 6.295 | 66.853 | 17.967 | 1.00 | 58.03 |
| 1610 | OH2 | TIP | A | 84 | 13.965 | 68.899 | −2.314 | 1.00 | 61.54 |
| 1611 | OH2 | TIP | A | 85 | 32.379 | 52.416 | −8.311 | 1.00 | 71.84 |
| 1612 | OH2 | TIP | A | 86 | 48.425 | 50.638 | 14.271 | 1.00 | 59.92 |
| 1613 | OH2 | TIP | A | 87 | 10.680 | 67.805 | 5.635 | 1.00 | 55.82 |
| 1614 | OH2 | TIP | A | 88 | 36.880 | 58.553 | 1.670 | 1.00 | 57.31 |
| 1615 | OH2 | TIP | A | 89 | 16.870 | 52.214 | 1.693 | 1.00 | 58.45 |
| 1616 | OH2 | TIP | A | 90 | 25.408 | 92.634 | 4.035 | 1.00 | 58.03 |
| 1617 | OH2 | TIP | A | 91 | 13.095 | 80.378 | 22.328 | 1.00 | 50.36 |
| 1618 | OH2 | TIP | A | 92 | 29.763 | 48.629 | 0.935 | 1.00 | 57.19 |
| 1619 | OH2 | TIP | A | 93 | 48.144 | 52.717 | 5.484 | 1.00 | 62.27 |
| 1620 | OH2 | TIP | A | 94 | 32.716 | 83.953 | 9.514 | 1.00 | 61.49 |
| 1621 | OH2 | TIP | A | 95 | 50.245 | 51.781 | 10.104 | 1.00 | 66.72 |
| 1622 | OH2 | TIP | A | 96 | 19.486 | 103.622 | 14.976 | 1.00 | 62.93 |
| 1623 | OH2 | TIP | A | 97 | 16.771 | 101.927 | 12.421 | 1.00 | 59.08 |
| 1624 | OH2 | TIP | A | 98 | 53.390 | 55.312 | 11.504 | 1.00 | 66.54 |
| 1625 | OH2 | TIP | A | 99 | 50.837 | 51.731 | 13.291 | 1.00 | 64.16 |
| 1626 | OH2 | TIP | A | 100 | 23.981 | 65.804 | −2.209 | 1.00 | 59.09 |
| 1627 | OH2 | TIP | A | 101 | 23.552 | 78.063 | 7.649 | 1.00 | 59.99 |
| 1628 | OH2 | TIP | A | 102 | 2.205 | 69.469 | 16.152 | 1.00 | 63.48 |
| 1629 | OH2 | TIP | A | 103 | 28.886 | 49.557 | 15.035 | 1.00 | 60.94 |
| 1630 | OH2 | TIP | A | 104 | 16.467 | 73.762 | 27.029 | 1.00 | 57.13 |
| 1631 | OH2 | TIP | A | 105 | 12.719 | 56.573 | 17.218 | 1.00 | 67.01 |
| 1632 | OH2 | TIP | A | 106 | 35.645 | 52.940 | 2.917 | 1.00 | 61.12 |
| 1633 | OH2 | TIP | A | 107 | 21.697 | 47.608 | 11.251 | 1.00 | 62.98 |
| 1634 | OH2 | TIP | A | 108 | 29.875 | 69.867 | 19.761 | 1.00 | 63.30 |
| 1635 | OH2 | TIP | A | 109 | 7.022 | 65.025 | 20.275 | 1.00 | 57.78 |
| 1636 | OH2 | TIP | A | 110 | 23.672 | 57.957 | 18.156 | 1.00 | 65.34 |
| 1637 | OH2 | TIP | A | 111 | 18.442 | 100.288 | 19.662 | 1.00 | 56.09 |
| 1638 | OH2 | TIP | A | 112 | 17.274 | 55.759 | 23.904 | 1.00 | 62.75 |
| 1639 | OH2 | TIP | A | 113 | 12.468 | 99.002 | 11.775 | 1.00 | 58.88 |
| 1640 | OH2 | TIP | A | 114 | 4.947 | 78.508 | 15.926 | 1.00 | 63.28 |
| 1641 | OH2 | TIP | A | 115 | 51.851 | 63.576 | 7.665 | 1.00 | 65.61 |
| 1642 | OH2 | TIP | A | 116 | 28.686 | 55.061 | 19.178 | 1.00 | 60.98 |
| 1643 | OH2 | TIP | A | 117 | 13.344 | 58.062 | 13.444 | 1.00 | 60.98 |
| 1644 | OH2 | TIP | A | 118 | 31.348 | 100.434 | 17.891 | 1.00 | 58.25 |
| 1645 | OH2 | TIP | A | 119 | 33.355 | 67.383 | 15.181 | 1.00 | 55.39 |
| 1646 | OH2 | TIP | A | 120 | 50.364 | 73.348 | 11.901 | 1.00 | 56.80 |
| 1647 | OH2 | TIP | A | 121 | 48.002 | 72.930 | 13.297 | 1.00 | 61.15 |
| 1648 | OH2 | TIP | A | 122 | 8.619 | 87.814 | −3.671 | 1.00 | 55.82 |
| 1649 | OH2 | TIP | A | 123 | 28.580 | 65.220 | 7.059 | 1.00 | 45.14 |
| 1650 | OH2 | TIP | A | 124 | 17.490 | 63.562 | 13.375 | 1.00 | 55.49 |
| 1651 | OH2 | TIP | A | 125 | 50.105 | 70.842 | 12.277 | 1.00 | 66.22 |
| 1652 | OH2 | TIP | A | 126 | 28.516 | 67.863 | 5.456 | 1.00 | 57.00 |
| 1653 | OH2 | TIP | A | 127 | 7.299 | 75.901 | 18.432 | 1.00 | 57.60 |
| 1654 | OH2 | TIP | A | 128 | 16.230 | 56.580 | 0.876 | 1.00 | 56.82 |
| 1655 | OH2 | TIP | A | 129 | 29.715 | 52.741 | 21.104 | 1.00 | 62.05 |
| 1656 | OH2 | TIP | A | 130 | 46.391 | 75.590 | 10.503 | 1.00 | 66.31 |
| 1657 | OH2 | TIP | A | 131 | 8.623 | 74.686 | 21.869 | 1.00 | 57.71 |
| 1658 | OH2 | TIP | A | 132 | 25.877 | 65.684 | −0.527 | 1.00 | 61.64 |
| 1659 | OH2 | TIP | A | 133 | 48.195 | 61.492 | 2.382 | 1.00 | 61.06 |
| 1660 | OH2 | TIP | A | 134 | 26.143 | 95.737 | 23.267 | 1.00 | 61.78 |
| 1661 | OH2 | TIP | A | 135 | 19.283 | 66.683 | 28.340 | 1.00 | 57.35 |
| 1662 | OH2 | TIP | A | 136 | 23.744 | 74.738 | 11.921 | 1.00 | 54.03 |
| 1663 | OH2 | TIP | A | 137 | 34.653 | 51.896 | −6.794 | 1.00 | 64.00 |
| 1664 | OH2 | TIP | A | 138 | 23.762 | 64.741 | 24.873 | 1.00 | 65.85 |
| 1665 | OH2 | TIP | A | 139 | 9.472 | 67.784 | 26.691 | 1.00 | 60.91 |
| 1666 | OH2 | TIP | A | 140 | 31.126 | 79.895 | 13.795 | 1.00 | 61.62 |
| 1667 | OH2 | TIP | A | 141 | 51.302 | 75.595 | 10.454 | 1.00 | 62.13 |
| 1668 | OH2 | TIP | A | 142 | 25.624 | 94.976 | 18.923 | 1.00 | 58.14 |
| 1669 | OH2 | TIP | A | 143 | 46.215 | 76.102 | 4.024 | 1.00 | 61.05 |
| 1670 | OH2 | TIP | A | 144 | 14.705 | 65.280 | 31.014 | 1.00 | 55.65 |
| 1671 | OH2 | TIP | A | 145 | 39.069 | 49.876 | 20.322 | 1.00 | 64.64 |
| 1672 | OH2 | TIP | A | 146 | 10.862 | 54.941 | 15.417 | 1.00 | 61.54 |
| 1673 | OH2 | TIP | A | 147 | 20.183 | 101.944 | 22.268 | 1.00 | 59.07 |
| 1674 | OH2 | TIP | A | 148 | 29.707 | 89.335 | 8.858 | 1.00 | 60.79 |
| 1675 | OH2 | TIP | A | 149 | 10.193 | 73.823 | 5.510 | 1.00 | 57.59 |
| 1676 | OH2 | TIP | A | 150 | 29.352 | 51.313 | −6.490 | 1.00 | 63.92 |
| 1677 | OH2 | TIP | A | 151 | 25.999 | 72.109 | 30.307 | 1.00 | 62.67 |
| 1678 | OH2 | TIP | A | 152 | 10.844 | 86.445 | −4.018 | 1.00 | 68.07 |
| 1679 | OH2 | TIP | A | 153 | 30.550 | 70.355 | 12.969 | 1.00 | 61.87 |
| 1680 | OH2 | TIP | A | 154 | 28.953 | 49.265 | 12.349 | 1.00 | 66.95 |
| 1681 | OH2 | TIP | A | 155 | 10.816 | 50.012 | 8.484 | 1.00 | 59.56 |
| 1682 | OH2 | TIP | A | 156 | 27.343 | 69.536 | 30.284 | 1.00 | 59.20 |
| 1683 | OH2 | TIP | A | 157 | 48.276 | 50.311 | 10.430 | 1.00 | 67.18 |
| 1684 | OH2 | TIP | A | 158 | 9.916 | 67.356 | 2.963 | 1.00 | 59.37 |
| 1685 | OH2 | TIP | A | 159 | 24.834 | 107.006 | 22.307 | 1.00 | 65.79 |
| 1686 | OH2 | TIP | A | 160 | 15.746 | 59.001 | 13.607 | 1.00 | 58.89 |
| 1687 | OH2 | TIP | A | 161 | 31.698 | 74.365 | 32.777 | 1.00 | 59.68 |
| 1688 | OH2 | TIP | A | 162 | 21.890 | 56.335 | −1.064 | 1.00 | 63.91 |
| 1689 | OH2 | TIP | A | 163 | 14.286 | 93.107 | 19.563 | 1.00 | 58.95 |
| 1690 | OH2 | TIP | A | 164 | 23.710 | 75.161 | 5.470 | 1.00 | 64.19 |
| 1691 | OH2 | TIP | A | 165 | 24.206 | 72.021 | 7.712 | 1.00 | 55.30 |
| 1692 | OH2 | TIP | A | 166 | 20.559 | 81.972 | −0.663 | 1.00 | 55.04 |
| 1693 | OH2 | TIP | A | 167 | 28.070 | 68.574 | 17.772 | 1.00 | 61.56 |
| 1694 | OH2 | TIP | A | 168 | 57.914 | 63.409 | 6.737 | 1.00 | 63.55 |
| 1695 | OH2 | TIP | A | 169 | 18.340 | 57.211 | 25.770 | 1.00 | 62.46 |
| 1696 | OH2 | TIP | A | 170 | 26.782 | 106.710 | 18.895 | 1.00 | 61.28 |
| 1697 | OH2 | TIP | A | 171 | 28.254 | 75.284 | 31.745 | 1.00 | 59.15 |
| 1698 | OH2 | TIP | A | 172 | 46.877 | 48.708 | 12.388 | 1.00 | 66.69 |
| 1699 | OH2 | TIP | A | 173 | 15.777 | 67.027 | −3.922 | 1.00 | 60.64 |
| 1700 | OH2 | TIP | A | 174 | 32.197 | 49.900 | 17.143 | 1.00 | 64.56 |
| 1701 | OH2 | TIP | A | 175 | 23.440 | 103.469 | 17.348 | 1.00 | 59.77 |
| 1702 | OH2 | TIP | A | 176 | 30.137 | 56.948 | 21.224 | 1.00 | 61.95 |
| 1703 | OH2 | TIP | A | 177 | 26.468 | 91.670 | 1.313 | 1.00 | 63.68 |
| 1704 | OH2 | TIP | A | 178 | 25.828 | 56.552 | 19.074 | 1.00 | 62.71 |
| 1705 | OH2 | TIP | A | 179 | 34.582 | 54.727 | 20.637 | 1.00 | 64.40 |
| 1706 | OH2 | TIP | A | 180 | 17.987 | 105.822 | 18.202 | 1.00 | 64.94 |
| 1707 | OH2 | TIP | A | 181 | 6.122 | 68.884 | 20.390 | 1.00 | 60.70 |
| 1708 | OH2 | TIP | A | 182 | 8.806 | 49.867 | 4.420 | 1.00 | 62.42 |
| 1709 | OH2 | TIP | A | 183 | 27.312 | 72.638 | 16.534 | 1.00 | 65.26 |
| 1710 | OH2 | TIP | A | 184 | 31.069 | 55.528 | 19.225 | 1.00 | 58.49 |
| 1711 | OH2 | TIP | A | 185 | 25.301 | 101.534 | 21.383 | 1.00 | 66.38 |
| 1712 | OH2 | TIP | A | 186 | 22.607 | 53.815 | 1.063 | 1.00 | 66.62 |
| 1713 | OH2 | TIP | A | 187 | 16.147 | 98.913 | 21.300 | 1.00 | 63.72 |
| 1714 | OH2 | TIP | A | 188 | 17.776 | 102.185 | 18.290 | 1.00 | 62.49 |
| 1715 | OH2 | TIP | A | 189 | 31.779 | 47.168 | 4.361 | 1.00 | 63.23 |
| 1716 | OH2 | TIP | A | 190 | 16.083 | 101.996 | 14.898 | 1.00 | 65.15 |
| 1717 | OH2 | TIP | A | 191 | 36.208 | 68.938 | 20.199 | 1.00 | 63.27 |
| 1718 | OH2 | TIP | A | 192 | 36.586 | 54.580 | 0.794 | 1.00 | 61.81 |
| 1719 | OH2 | TIP | A | 193 | 32.810 | 70.773 | 20.047 | 1.00 | 59.78 |
| 1720 | OH2 | TIP | A | 194 | 7.956 | 66.621 | 5.340 | 1.00 | 60.91 |
| 1721 | OH2 | TIP | A | 195 | 16.254 | 80.162 | 0.421 | 1.00 | 61.51 |
| 1722 | CB | VAL | B | 336 | 45.054 | 59.383 | 30.496 | 1.00 | 63.58 |
| 1723 | CG1 | VAL | B | 336 | 45.744 | 60.010 | 31.695 | 1.00 | 64.78 |
| 1724 | CG2 | VAL | B | 336 | 44.712 | 57.923 | 30.765 | 1.00 | 68.51 |
| 1725 | C | VAL | B | 336 | 44.164 | 61.555 | 29.666 | 1.00 | 61.62 |
| 1726 | O | VAL | B | 336 | 45.201 | 61.720 | 29.026 | 1.00 | 64.02 |
| 1727 | N | VAL | B | 336 | 42.976 | 59.439 | 29.128 | 1.00 | 59.58 |
| 1728 | CA | VAL | B | 336 | 43.773 | 60.162 | 30.157 | 1.00 | 61.95 |
| 1729 | N | SER | B | 337 | 43.338 | 62.554 | 29.963 | 1.00 | 59.54 |
| 1730 | CA | SER | B | 337 | 43.618 | 63.921 | 29.536 | 1.00 | 57.94 |
| 1731 | CB | SER | B | 337 | 42.531 | 64.396 | 28.575 | 1.00 | 56.70 |
| 1732 | OG | SER | B | 337 | 41.253 | 64.019 | 29.047 | 1.00 | 65.28 |
| 1733 | C | SER | B | 337 | 43.767 | 64.903 | 30.700 | 1.00 | 56.67 |
| 1734 | O | SER | B | 337 | 43.366 | 64.613 | 31.832 | 1.00 | 56.61 |
| 1735 | N | ALA | B | 338 | 44.355 | 66.061 | 30.413 | 1.00 | 52.92 |
| 1736 | CA | ALA | B | 338 | 44.589 | 67.079 | 31.429 | 1.00 | 51.01 |
| 1737 | CB | ALA | B | 338 | 46.038 | 67.024 | 31.884 | 1.00 | 50.96 |
| 1738 | C | ALA | B | 338 | 44.252 | 68.480 | 30.934 | 1.00 | 50.95 |
| 1739 | O | ALA | B | 338 | 44.396 | 68.784 | 29.748 | 1.00 | 53.16 |
| 1740 | N | TYR | B | 339 | 43.795 | 69.328 | 31.850 | 1.00 | 47.68 |
| 1741 | CA | TYR | B | 339 | 43.431 | 70.697 | 31.508 | 1.00 | 48.08 |
| 1742 | CB | TYR | B | 339 | 41.925 | 70.790 | 31.203 | 1.00 | 50.34 |
| 1743 | CG | TYR | B | 339 | 41.382 | 69.708 | 30.283 | 1.00 | 58.74 |
| 1744 | CD1 | TYR | B | 339 | 41.179 | 68.404 | 30.745 | 1.00 | 60.22 |
| 1745 | CE1 | TYR | B | 339 | 40.705 | 67.401 | 29.895 | 1.00 | 64.78 |
| 1746 | CD2 | TYR | B | 339 | 41.094 | 69.984 | 28.947 | 1.00 | 60.49 |
| 1747 | CE2 | TYR | B | 339 | 40.620 | 68.992 | 28.088 | 1.00 | 63.00 |
| 1748 | CZ | TYR | B | 339 | 40.428 | 67.700 | 28.567 | 1.00 | 67.92 |
| 1749 | OH | TYR | B | 339 | 39.974 | 66.709 | 27.718 | 1.00 | 68.47 |
| 1750 | C | TYR | B | 339 | 43.785 | 71.646 | 32.663 | 1.00 | 45.49 |
| 1751 | O | TYR | B | 339 | 43.556 | 71.338 | 33.833 | 1.00 | 43.52 |
| 1752 | N | LEU | B | 340 | 44.362 | 72.792 | 32.325 | 1.00 | 44.01 |
| 1753 | CA | LEU | B | 340 | 44.728 | 73.784 | 33.324 | 1.00 | 42.54 |
| 1754 | CB | LEU | B | 340 | 46.237 | 74.050 | 33.284 | 1.00 | 39.07 |
| 1755 | CG | LEU | B | 340 | 46.850 | 74.948 | 34.370 | 1.00 | 39.58 |
| 1756 | CD1 | LEU | B | 340 | 46.526 | 74.402 | 35.751 | 1.00 | 28.76 |
| 1757 | CD2 | LEU | B | 340 | 48.364 | 75.029 | 34.177 | 1.00 | 35.57 |
| 1758 | C | LEU | B | 340 | 43.955 | 75.043 | 32.966 | 1.00 | 42.53 |
| 1759 | O | LEU | B | 340 | 43.962 | 75.462 | 31.814 | 1.00 | 43.25 |
| 1760 | N | SER | B | 341 | 43.276 | 75.643 | 33.940 | 1.00 | 42.42 |
| 1761 | CA | SER | B | 341 | 42.497 | 76.848 | 33.675 | 1.00 | 40.32 |
| 1762 | CB | SER | B | 341 | 41.014 | 76.603 | 33.995 | 1.00 | 41.30 |
| 1763 | OG | SER | B | 341 | 40.842 | 76.184 | 35.336 | 1.00 | 45.86 |
| 1764 | C | SER | B | 341 | 42.994 | 78.037 | 34.466 | 1.00 | 38.67 |
| 1765 | O | SER | B | 341 | 43.552 | 77.893 | 35.541 | 1.00 | 42.94 |
| 1766 | N | ARG | B | 342 | 42.777 | 79.222 | 33.923 | 1.00 | 37.60 |
| 1767 | CA | ARG | B | 342 | 43.186 | 80.445 | 34.580 | 1.00 | 35.91 |
| 1768 | CB | ARG | B | 342 | 43.241 | 81.574 | 33.548 | 1.00 | 36.67 |
| 1769 | OG | ARG | B | 342 | 44.285 | 81.346 | 32.454 | 1.00 | 39.89 |
| 1770 | CD | ARG | B | 342 | 44.278 | 82.459 | 31.421 | 1.00 | 42.31 |
| 1771 | NE | ARG | B | 342 | 43.066 | 82.415 | 30.610 | 1.00 | 45.87 |
| 1772 | CZ | ARG | B | 342 | 42.870 | 81.595 | 29.580 | 1.00 | 52.30 |
| 1773 | NE1 | ARG | B | 342 | 43.810 | 80.739 | 29.205 | 1.00 | 56.74 |
| 1774 | NH2 | ARG | B | 342 | 41.715 | 81.619 | 28.932 | 1.00 | 58.22 |
| 1775 | C | ARG | B | 342 | 42.191 | 80.768 | 35.702 | 1.00 | 34.19 |
| 1776 | O | ARG | B | 342 | 41.150 | 80.124 | 35.822 | 1.00 | 34.44 |
| 1777 | N | PRO | B | 343 | 42.499 | 81.763 | 36.545 | 1.00 | 31.22 |
| 1778 | CD | PRO | B | 343 | 43.729 | 82.572 | 36.606 | 1.00 | 34.08 |
| 1779 | CA | PRO | B | 343 | 41.576 | 82.114 | 37.634 | 1.00 | 33.86 |
| 1780 | CB | PRO | B | 343 | 42.356 | 83.163 | 38.437 | 1.00 | 32.10 |
| 1781 | CG | PRO | B | 343 | 43.811 | 82.902 | 38.078 | 1.00 | 37.07 |
| 1782 | C | PRO | B | 343 | 40.277 | 82.703 | 37.085 | 1.00 | 32.54 |
| 1783 | O | PRO | B | 343 | 40.281 | 83.330 | 36.028 | 1.00 | 35.02 |
| 1784 | N | SER | B | 344 | 39.168 | 82.510 | 37.789 | 1.00 | 33.04 |
| 1785 | CA | SER | B | 344 | 37.913 | 83.085 | 37.322 | 1.00 | 34.74 |
| 1786 | CB | SER | B | 344 | 36.703 | 82.428 | 38.005 | 1.00 | 35.18 |
| 1787 | OG | SER | B | 344 | 36.507 | 82.937 | 39.308 | 1.00 | 35.70 |
| 1788 | C | SER | B | 344 | 37.972 | 84.562 | 37.685 | 1.00 | 35.17 |
| 1789 | O | SER | B | 344 | 38.468 | 84.931 | 38.751 | 1.00 | 32.53 |
| 1790 | N | PRO | B | 345 | 37.506 | 85.438 | 36.787 | 1.00 | 35.98 |
| 1791 | CD | PRO | B | 345 | 37.180 | 85.223 | 35.364 | 1.00 | 36.93 |
| 1792 | CA | PRO | B | 345 | 37.544 | 86.869 | 37.105 | 1.00 | 34.79 |
| 1793 | CB | PRO | B | 345 | 36.870 | 87.503 | 35.888 | 1.00 | 30.45 |
| 1794 | CG | PRO | B | 345 | 37.379 | 86.612 | 34.769 | 1.00 | 30.91 |
| 1795 | C | PRO | B | 345 | 36.861 | 87.215 | 38.433 | 1.00 | 34.13 |
| 1796 | O | PRO | B | 345 | 37.283 | 88.129 | 39.137 | 1.00 | 34.14 |
| 1797 | N | PHE | B | 346 | 35.817 | 86.476 | 38.791 | 1.00 | 31.57 |
| 1798 | CA | PHE | B | 346 | 35.134 | 86.752 | 40.041 | 1.00 | 33.19 |
| 1799 | CB | PHE | B | 346 | 33.876 | 85.889 | 40.165 | 1.00 | 37.49 |
| 1800 | CG | PHE | B | 346 | 33.206 | 85.984 | 41.505 | 1.00 | 38.65 |
| 1801 | CD1 | PHE | B | 346 | 32.621 | 87.168 | 41.922 | 1.00 | 42.62 |
| 1802 | CD2 | PHE | B | 346 | 33.159 | 84.881 | 42.353 | 1.00 | 43.21 |
| 1803 | CE1 | PHE | B | 346 | 31.992 | 87.256 | 43.167 | 1.00 | 41.72 |
| 1804 | CE2 | PHE | B | 346 | 32.531 | 84.957 | 43.604 | 1.00 | 49.00 |
| 1805 | CZ | PHE | B | 346 | 31.946 | 86.148 | 44.009 | 1.00 | 42.82 |
| 1806 | C | PHE | B | 346 | 36.070 | 86.501 | 41.233 | 1.00 | 33.37 |
| 1807 | O | PHE | B | 346 | 36.143 | 87.316 | 42.152 | 1.00 | 33.52 |
| 1808 | N | ASP | B | 347 | 36.790 | 85.384 | 41.216 | 1.00 | 32.94 |
| 1809 | CA | ASP | B | 347 | 37.709 | 85.067 | 42.308 | 1.00 | 33.61 |
| 1810 | CB | ASP | B | 347 | 38.242 | 83.638 | 42.173 | 1.00 | 29.68 |
| 1811 | CG | ASP | B | 347 | 37.222 | 82.589 | 42.559 | 1.00 | 35.85 |
| 1812 | OD1 | ASP | B | 347 | 37.516 | 81.386 | 42.382 | 1.00 | 42.71 |
| 1813 | OD2 | ASP | B | 347 | 36.128 | 82.954 | 43.044 | 1.00 | 50.58 |
| 1814 | C | ASP | B | 347 | 38.882 | 86.036 | 42.338 | 1.00 | 33.62 |
| 1815 | O | ASP | B | 347 | 39.381 | 86.399 | 43.401 | 1.00 | 30.10 |
| 1816 | N | LEU | B | 348 | 39.312 | 86.463 | 41.160 | 1.00 | 34.23 |
| 1817 | CA | LEU | B | 348 | 40.437 | 87.374 | 41.046 | 1.00 | 38.75 |
| 1818 | CB | LEU | B | 348 | 40.964 | 87.340 | 39.604 | 1.00 | 38.06 |
| 1819 | CG | LEU | B | 348 | 42.165 | 88.237 | 39.289 | 1.00 | 43.84 |
| 1820 | CD1 | LEU | B | 348 | 43.357 | 87.861 | 40.190 | 1.00 | 39.77 |
| 1821 | CD2 | LEU | B | 348 | 42.536 | 88.090 | 37.809 | 1.00 | 43.35 |
| 1822 | C | LEU | B | 348 | 40.161 | 88.837 | 41.455 | 1.00 | 39.57 |
| 1823 | O | LEU | B | 348 | 40.979 | 89.454 | 42.141 | 1.00 | 38.51 |
| 1824 | N | PHE | B | 349 | 39.017 | 89.386 | 41.043 | 1.00 | 40.41 |
| 1825 | CA | PHE | B | 349 | 38.699 | 90.791 | 41.338 | 1.00 | 42.87 |
| 1826 | CB | PHE | B | 349 | 38.236 | 91.489 | 40.060 | 1.00 | 39.59 |
| 1827 | CG | PHE | B | 349 | 39.217 | 91.392 | 38.937 | 1.00 | 37.95 |
| 1828 | CD1 | PHE | B | 349 | 38.951 | 90.596 | 37.832 | 1.00 | 36.40 |
| 1829 | CD2 | PHE | B | 349 | 40.419 | 92.083 | 38.995 | 1.00 | 40.49 |
| 1830 | CE1 | PHE | B | 349 | 39.874 | 90.484 | 36.795 | 1.00 | 39.63 |
| 1831 | CE2 | PHE | B | 349 | 41.353 | 91.981 | 37.965 | 1.00 | 38.12 |
| 1832 | CZ | PHE | B | 349 | 41.080 | 91.182 | 36.863 | 1.00 | 41.86 |
| 1833 | C | PHE | B | 349 | 37.704 | 91.128 | 42.446 | 1.00 | 44.67 |
| 1834 | O | PHE | B | 349 | 37.827 | 92.167 | 43.099 | 1.00 | 47.25 |
| 1835 | N | ILE | B | 350 | 36.705 | 90.286 | 42.650 | 1.00 | 46.06 |
| 1836 | CA | ILE | B | 350 | 35.729 | 90.573 | 43.683 | 1.00 | 50.35 |
| 1837 | CB | ILE | B | 350 | 34.330 | 90.052 | 43.287 | 1.00 | 50.30 |
| 1838 | CG2 | ILE | B | 350 | 33.284 | 90.603 | 44.236 | 1.00 | 51.91 |
| 1839 | CG1 | ILE | B | 350 | 34.007 | 90.454 | 41.839 | 1.00 | 51.78 |
| 1840 | CD1 | ILE | B | 350 | 34.234 | 91.915 | 41.520 | 1.00 | 45.73 |
| 1841 | C | ILE | B | 350 | 36.179 | 89.906 | 44.973 | 1.00 | 51.95 |
| 1842 | O | ILE | B | 350 | 36.454 | 90.572 | 45.970 | 1.00 | 52.71 |
| 1843 | N | ARG | B | 351 | 36.281 | 88.584 | 44.927 | 1.00 | 53.39 |
| 1844 | CA | ARG | B | 351 | 36.691 | 87.787 | 46.069 | 1.00 | 54.67 |
| 1845 | CB | ARG | B | 351 | 36.541 | 86.310 | 45.707 | 1.00 | 59.14 |
| 1846 | CG | ARG | B | 351 | 36.063 | 85.414 | 46.833 | 1.00 | 64.87 |
| 1847 | CD | ARG | B | 351 | 35.127 | 84.346 | 46.292 | 1.00 | 66.51 |
| 1848 | NE | ARG | B | 351 | 35.034 | 83.195 | 47.180 | 1.00 | 73.42 |
| 1849 | CZ | ARG | B | 351 | 36.027 | 82.337 | 47.383 | 1.00 | 78.26 |
| 1850 | NH1 | ARG | B | 351 | 37.183 | 82.506 | 46.756 | 1.00 | 81.30 |
| 1851 | NH2 | ARG | B | 351 | 35.867 | 81.312 | 48.211 | 1.00 | 79.63 |
| 1852 | C | ARG | B | 351 | 38.132 | 88.092 | 46.484 | 1.00 | 53.92 |
| 1853 | O | ARG | B | 351 | 38.456 | 88.090 | 47.667 | 1.00 | 53.66 |
| 1854 | N | LYS | B | 352 | 38.988 | 88.352 | 45.502 | 1.00 | 53.41 |
| 1855 | CA | LYS | B | 352 | 40.400 | 88.658 | 45.740 | 1.00 | 53.02 |
| 1856 | CB | LYS | B | 352 | 40.547 | 89.821 | 46.725 | 1.00 | 54.25 |
| 1857 | CG | LYS | B | 352 | 39.848 | 91.094 | 46.286 | 1.00 | 64.61 |
| 1858 | CD | LYS | B | 352 | 40.158 | 92.234 | 47.246 | 1.00 | 71.92 |
| 1859 | CE | LYS | B | 352 | 39.190 | 93.398 | 47.086 | 1.00 | 76.75 |
| 1860 | NZ | LYS | B | 352 | 37.807 | 93.027 | 47.505 | 1.00 | 82.42 |
| 1861 | C | LYS | B | 352 | 41.217 | 87.470 | 46.253 | 1.00 | 51.14 |
| 1862 | O | LYS | B | 352 | 42.183 | 87.655 | 46.991 | 1.00 | 51.55 |
| 1863 | N | SER | B | 353 | 40.828 | 86.259 | 45.863 | 1.00 | 47.69 |
| 1864 | CA | SER | B | 353 | 41.533 | 85.048 | 46.268 | 1.00 | 45.50 |
| 1865 | CB | SER | B | 353 | 40.882 | 84.446 | 47.518 | 1.00 | 50.28 |
| 1866 | OG | SER | B | 353 | 39.542 | 84.062 | 47.271 | 1.00 | 63.45 |
| 1867 | C | SER | B | 353 | 41.465 | 84.072 | 45.094 | 1.00 | 40.39 |
| 1868 | O | SER | B | 353 | 40.710 | 83.102 | 45.105 | 1.00 | 40.20 |
| 1869 | N | PRO | B | 354 | 42.271 | 84.332 | 44.055 | 1.00 | 36.74 |
| 1870 | CD | PRO | B | 354 | 43.262 | 85.412 | 44.061 | 1.00 | 36.52 |
| 1871 | CA | PRO | B | 354 | 42.376 | 83.557 | 42.817 | 1.00 | 34.20 |
| 1872 | CB | PRO | B | 354 | 43.283 | 84.418 | 41.937 | 1.00 | 31.70 |
| 1873 | CG | PRO | B | 354 | 43.340 | 85.730 | 42.628 | 1.00 | 40.26 |
| 1874 | C | PRO | B | 354 | 42.964 | 82.167 | 42.979 | 1.00 | 32.13 |
| 1875 | O | PRO | B | 354 | 43.829 | 81.945 | 43.817 | 1.00 | 28.54 |
| 1876 | N | THR | B | 355 | 42.489 | 81.241 | 42.158 | 1.00 | 30.96 |
| 1877 | CA | THR | B | 355 | 42.999 | 79.882 | 42.164 | 1.00 | 31.86 |
| 1878 | CB | THR | B | 355 | 42.099 | 78.908 | 42.979 | 1.00 | 32.67 |
| 1879 | OG1 | THR | B | 355 | 40.823 | 78.794 | 42.348 | 1.00 | 34.86 |
| 1880 | CG2 | THR | B | 355 | 41.894 | 79.406 | 44.401 | 1.00 | 34.90 |
| 1881 | C | THR | B | 355 | 43.052 | 79.379 | 40.726 | 1.00 | 33.38 |
| 1882 | O | THR | B | 355 | 42.361 | 79.897 | 39.840 | 1.00 | 32.61 |
| 1883 | N | ILE | B | 356 | 43.909 | 78.403 | 40.482 | 1.00 | 32.83 |
| 1884 | CA | ILE | B | 356 | 43.959 | 77.807 | 39.165 | 1.00 | 33.21 |
| 1885 | CB | ILE | B | 356 | 45.297 | 78.062 | 38.434 | 1.00 | 34.88 |
| 1886 | CG2 | ILE | B | 356 | 45.458 | 79.562 | 38.190 | 1.00 | 34.84 |
| 1887 | CG1 | ILE | B | 356 | 46.465 | 77.502 | 39.240 | 1.00 | 37.26 |
| 1888 | CD1 | ILE | B | 356 | 47.843 | 77.791 | 38.611 | 1.00 | 38.06 |
| 1889 | C | ILE | B | 356 | 43.745 | 76.335 | 39.461 | 1.00 | 32.48 |
| 1890 | O | ILE | B | 356 | 44.037 | 75.861 | 40.564 | 1.00 | 30.30 |
| 1891 | N | THR | B | 357 | 43.236 | 75.609 | 38.478 | 1.00 | 33.59 |
| 1892 | CA | THR | B | 357 | 42.929 | 74.211 | 38.694 | 1.00 | 32.83 |
| 1893 | CB | THR | B | 357 | 41.384 | 74.025 | 38.857 | 1.00 | 37.46 |
| 1894 | OG1 | THR | B | 357 | 40.955 | 74.615 | 40.091 | 1.00 | 35.11 |
| 1895 | CG2 | THR | B | 357 | 41.001 | 72.553 | 38.842 | 1.00 | 40.13 |
| 1896 | C | THR | B | 357 | 43.420 | 73.326 | 37.570 | 1.00 | 34.22 |
| 1897 | O | THR | B | 357 | 43.222 | 73.609 | 36.384 | 1.00 | 33.99 |
| 1898 | N | CYS | B | 358 | 44.045 | 72.231 | 37.966 | 1.00 | 34.04 |
| 1899 | CA | CYS | B | 358 | 44.563 | 71.256 | 37.032 | 1.00 | 36.90 |
| 1900 | C | CYS | B | 358 | 43.573 | 70.104 | 37.101 | 1.00 | 36.26 |
| 1901 | O | CYS | B | 358 | 43.412 | 69.497 | 38.156 | 1.00 | 35.68 |
| 1902 | CB | CYS | B | 358 | 45.930 | 70.791 | 37.507 | 1.00 | 39.83 |
| 1903 | SG | CYS | B | 358 | 46.881 | 69.819 | 36.302 | 1.00 | 52.73 |
| 1904 | N | LEU | B | 359 | 42.908 | 69.820 | 35.988 | 1.00 | 38.57 |
| 1905 | CA | LEU | B | 359 | 41.921 | 68.751 | 35.922 | 1.00 | 42.35 |
| 1906 | CB | LEU | B | 359 | 40.615 | 69.273 | 35.304 | 1.00 | 42.11 |
| 1907 | CG | LEU | B | 359 | 39.572 | 68.227 | 34.881 | 1.00 | 48.42 |
| 1908 | CD1 | LEU | B | 359 | 39.146 | 67.379 | 36.073 | 1.00 | 49.46 |
| 1909 | CD2 | LEU | B | 359 | 38.370 | 68.934 | 34.269 | 1.00 | 48.09 |
| 1910 | C | LEU | B | 359 | 42.437 | 67.589 | 35.095 | 1.00 | 45.39 |
| 1911 | O | LEU | B | 359 | 42.794 | 67.766 | 33.935 | 1.00 | 48.73 |
| 1912 | N | VAL | B | 360 | 42.479 | 66.405 | 35.693 | 1.00 | 47.72 |
| 1913 | CA | VAL | B | 360 | 42.931 | 65.205 | 34.996 | 1.00 | 51.87 |
| 1914 | CB | VAL | B | 360 | 44.075 | 64.514 | 35.765 | 1.00 | 52.99 |
| 1915 | CG1 | VAL | B | 360 | 44.490 | 63.235 | 35.051 | 1.00 | 51.29 |
| 1916 | CG2 | VAL | B | 360 | 45.252 | 65.456 | 35.890 | 1.00 | 52.42 |
| 1917 | C | VAL | B | 360 | 41.767 | 64.221 | 34.874 | 1.00 | 55.21 |
| 1918 | O | VAL | B | 360 | 41.063 | 63.966 | 35.853 | 1.00 | 56.01 |
| 1919 | N | VAL | B | 361 | 41.558 | 63.680 | 33.677 | 1.00 | 57.47 |
| 1920 | CA | VAL | B | 361 | 40.479 | 62.718 | 33.448 | 1.00 | 61.75 |
| 1921 | CB | VAL | B | 361 | 39.447 | 63.248 | 32.425 | 1.00 | 58.91 |
| 1922 | CG1 | VAL | B | 361 | 38.368 | 62.211 | 32.187 | 1.00 | 56.98 |
| 1923 | CG2 | VAL | B | 361 | 38.826 | 64.530 | 32.934 | 1.00 | 54.49 |
| 1924 | C | VAL | B | 361 | 41.045 | 61.403 | 32.923 | 1.00 | 66.78 |
| 1925 | O | VAL | B | 361 | 41.836 | 61.392 | 31.981 | 1.00 | 67.30 |
| 1926 | N | ASP | B | 362 | 40.641 | 60.299 | 33.544 | 1.00 | 72.35 |
| 1927 | CA | ASP | B | 362 | 41.100 | 58.969 | 33.145 | 1.00 | 79.16 |
| 1928 | CB | ASP | B | 362 | 41.775 | 58.266 | 34.331 | 1.00 | 83.66 |
| 1929 | CG | ASP | B | 362 | 42.511 | 56.997 | 33.924 | 1.00 | 87.40 |
| 1930 | OD1 | ASP | B | 362 | 42.317 | 56.529 | 32.780 | 1.00 | 91.40 |
| 1931 | OD2 | ASP | B | 362 | 43.282 | 56.462 | 34.752 | 1.00 | 87.33 |
| 1932 | C | ASP | B | 362 | 39.896 | 58.156 | 32.676 | 1.00 | 82.84 |
| 1933 | O | ASP | B | 362 | 39.113 | 57.663 | 33.493 | 1.00 | 84.14 |
| 1934 | N | ALA | B | 363 | 39.751 | 58.022 | 31.360 | 1.00 | 85.70 |
| 1935 | CA | ALA | B | 363 | 38.636 | 57.281 | 30.775 | 1.00 | 89.05 |
| 1936 | CB | ALA | B | 363 | 38.830 | 57.154 | 29.270 | 1.00 | 90.18 |
| 1937 | C | ALA | B | 363 | 38.458 | 55.899 | 31.401 | 1.00 | 91.80 |
| 1938 | O | ALA | B | 363 | 37.354 | 55.357 | 31.409 | 1.00 | 92.23 |
| 1939 | N | ALA | B | 364 | 39.544 | 55.337 | 31.926 | 1.00 | 94.23 |
| 1940 | CA | ALA | B | 364 | 39.515 | 54.022 | 32.564 | 1.00 | 97.42 |
| 1941 | CB | ALA | B | 364 | 39.441 | 52.932 | 31.506 | 1.00 | 96.05 |
| 1942 | C | ALA | B | 364 | 40.772 | 53.845 | 33.413 | 1.00 | 100.00 |
| 1943 | O | ALA | B | 364 | 41.885 | 53.833 | 32.887 | 1.00 | 101.29 |
| 1944 | N | PRO | B | 365 | 40.612 | 53.715 | 34.741 | 1.00 | 101.76 |
| 1945 | CD | PRO | B | 365 | 39.442 | 54.193 | 35.503 | 1.00 | 101.62 |
| 1946 | CA | PRO | B | 365 | 41.764 | 53.547 | 35.632 | 1.00 | 103.14 |
| 1947 | CB | PRO | B | 365 | 41.486 | 54.578 | 36.706 | 1.00 | 103.27 |
| 1948 | CG | PRO | B | 365 | 40.006 | 54.372 | 36.917 | 1.00 | 103.12 |
| 1949 | C | PRO | B | 365 | 41.919 | 52.144 | 36.228 | 1.00 | 104.03 |
| 1950 | O | PRO | B | 365 | 42.228 | 51.181 | 35.521 | 1.00 | 104.47 |
| 1951 | N | ALA | B | 366 | 41.716 | 52.064 | 37.543 | 1.00 | 104.37 |
| 1952 | CA | ALA | B | 366 | 41.807 | 50.824 | 38.307 | 1.00 | 104.87 |
| 1953 | CB | ALA | B | 366 | 40.895 | 49.761 | 37.687 | 1.00 | 105.36 |
| 1954 | C | ALA | B | 366 | 43.230 | 50.281 | 38.435 | 1.00 | 104.90 |
| 1955 | O | ALA | B | 366 | 43.492 | 49.139 | 38.055 | 1.00 | 105.14 |
| 1956 | N | LYS | B | 367 | 44.148 | 51.086 | 38.971 | 1.00 | 104.05 |
| 1957 | CA | LYS | B | 367 | 45.523 | 50.618 | 39.128 | 1.00 | 103.07 |
| 1958 | CB | LYS | B | 367 | 46.061 | 50.117 | 37.785 | 1.00 | 103.33 |
| 1959 | CG | LYS | B | 367 | 46.194 | 51.189 | 36.717 | 1.00 | 102.12 |
| 1960 | CD | LYS | B | 367 | 46.962 | 50.649 | 35.528 | 1.00 | 103.99 |
| 1961 | CE | LYS | B | 367 | 47.229 | 51.724 | 34.496 | 1.00 | 103.83 |
| 1962 | NZ | LYS | B | 367 | 48.056 | 51.183 | 33.386 | 1.00 | 105.88 |
| 1963 | C | LYS | B | 367 | 46.554 | 51.576 | 39.727 | 1.00 | 102.12 |
| 1964 | O | LYS | B | 367 | 47.653 | 51.712 | 39.185 | 1.00 | 103.05 |
| 1965 | N | GLY | B | 368 | 46.228 | 52.235 | 40.835 | 1.00 | 99.91 |
| 1966 | CA | OLY | B | 368 | 47.212 | 53.120 | 41.436 | 1.00 | 96.70 |
| 1967 | C | GLY | B | 368 | 46.800 | 54.521 | 41.845 | 1.00 | 94.03 |
| 1968 | O | GLY | B | 368 | 45.651 | 54.771 | 42.209 | 1.00 | 94.76 |
| 1969 | N | ALA | B | 369 | 47.761 | 55.438 | 41.787 | 1.00 | 90.44 |
| 1970 | CA | ALA | B | 369 | 47.534 | 56.828 | 42.165 | 1.00 | 86.18 |
| 1971 | CB | ALA | B | 369 | 48.292 | 57.140 | 43.455 | 1.00 | 86.97 |
| 1972 | C | ALA | B | 369 | 47.961 | 57.792 | 41.062 | 1.00 | 82.79 |
| 1973 | O | ALA | B | 369 | 48.773 | 57.451 | 40.203 | 1.00 | 83.46 |
| 1974 | N | VAL | B | 370 | 47.407 | 59.000 | 41.099 | 1.00 | 78.82 |
| 1975 | CA | VAL | B | 370 | 47.719 | 60.033 | 40.115 | 1.00 | 73.44 |
| 1976 | CB | VAL | B | 370 | 46.468 | 60.439 | 39.323 | 1.00 | 71.37 |
| 1977 | CG1 | VAL | B | 370 | 46.837 | 61.455 | 38.260 | 1.00 | 68.72 |
| 1978 | CG2 | VAL | B | 370 | 45.831 | 59.212 | 38.696 | 1.00 | 66.32 |
| 1979 | C | VAL | B | 370 | 48.254 | 61.253 | 40.848 | 1.00 | 71.19 |
| 1980 | O | VAL | B | 370 | 47.581 | 61.797 | 41.717 | 1.00 | 70.77 |
| 1981 | N | ASN | B | 371 | 49.462 | 61.687 | 40.495 | 1.00 | 69.11 |
| 1982 | CA | ASN | B | 371 | 50.069 | 62.831 | 41.164 | 1.00 | 67.35 |
| 1983 | CB | ASN | B | 371 | 51.462 | 62.455 | 41.670 | 1.00 | 68.95 |
| 1984 | CG | ASN | B | 371 | 51.432 | 61.270 | 42.602 | 1.00 | 73.37 |
| 1985 | OD1 | ASN | B | 371 | 50.772 | 61.301 | 43.641 | 1.00 | 76.52 |
| 1986 | ND2 | ASN | B | 371 | 52.146 | 60.213 | 42.237 | 1.00 | 76.06 |
| 1987 | C | ASN | B | 371 | 50.159 | 64.109 | 40.334 | 1.00 | 65.35 |
| 1988 | O | ASN | B | 371 | 50.454 | 64.084 | 39.136 | 1.00 | 63.63 |
| 1989 | N | LEU | B | 372 | 49.892 | 65.227 | 41.000 | 1.00 | 63.51 |
| 1990 | CA | LEU | B | 372 | 49.949 | 66.540 | 40.379 | 1.00 | 60.50 |
| 1991 | CB | LEU | B | 372 | 48.573 | 67.214 | 40.395 | 1.00 | 60.09 |
| 1992 | CG | LEU | B | 372 | 47.430 | 66.461 | 39.714 | 1.00 | 61.37 |
| 1993 | CD1 | LEU | B | 372 | 46.196 | 67.335 | 39.688 | 1.00 | 63.12 |
| 1994 | CD2 | LEU | B | 372 | 47.829 | 66.083 | 38.309 | 1.00 | 62.53 |
| 1995 | C | LEU | B | 372 | 50.942 | 67.380 | 41.164 | 1.00 | 57.54 |
| 1996 | O | LEU | B | 372 | 50.824 | 67.531 | 42.380 | 1.00 | 58.06 |
| 1997 | N | THR | B | 373 | 51.926 | 67.919 | 40.458 | 1.00 | 54.64 |
| 1998 | CA | THR | B | 373 | 52.952 | 68.745 | 41.074 | 1.00 | 52.59 |
| 1999 | CB | THR | B | 373 | 54.346 | 68.175 | 40.786 | 1.00 | 54.37 |
| 2000 | OG1 | THR | B | 373 | 54.383 | 66.801 | 41.191 | 1.00 | 60.15 |
| 2001 | CG2 | THR | B | 373 | 55.409 | 68.955 | 41.545 | 1.00 | 57.66 |
| 2002 | C | THR | B | 373 | 52.878 | 70.156 | 40.509 | 1.00 | 48.12 |
| 2003 | O | THR | B | 373 | 52.803 | 70.337 | 39.297 | 1.00 | 46.78 |
| 2004 | N | TRP | B | 374 | 52.895 | 71.150 | 41.391 | 1.00 | 45.42 |
| 2005 | CA | TRP | B | 374 | 52.833 | 72.543 | 40.971 | 1.00 | 44.33 |
| 2006 | CB | TRP | B | 374 | 51.838 | 73.340 | 41.823 | 1.00 | 40.42 |
| 2007 | CG | TRP | B | 374 | 50.417 | 72.936 | 41.658 | 1.00 | 41.22 |
| 2008 | CD2 | TRP | B | 374 | 49.510 | 73.385 | 40.643 | 1.00 | 35.68 |
| 2009 | CE2 | TRP | B | 374 | 48.271 | 72.759 | 40.881 | 1.00 | 31.67 |
| 2010 | CE3 | TRP | B | 374 | 49.628 | 74.258 | 39.555 | 1.00 | 35.08 |
| 2011 | CD1 | TRP | B | 374 | 49.712 | 72.077 | 42.448 | 1.00 | 38.77 |
| 2012 | NE1 | TRP | B | 374 | 48.420 | 71.966 | 41.988 | 1.00 | 37.27 |
| 2013 | CZ2 | TRP | B | 374 | 47.153 | 72.979 | 40.073 | 1.00 | 32.53 |
| 2014 | CZ3 | TRP | B | 374 | 48.516 | 74.479 | 38.748 | 1.00 | 36.03 |
| 2015 | CH2 | TRP | B | 374 | 47.296 | 73.840 | 39.013 | 1.00 | 33.34 |
| 2016 | C | TRP | B | 374 | 54.195 | 73.215 | 41.078 | 1.00 | 44.84 |
| 2017 | O | TRP | B | 374 | 55.019 | 72.842 | 41.914 | 1.00 | 43.54 |
| 2018 | N | SER | B | 375 | 54.419 | 74.205 | 40.218 | 1.00 | 44.41 |
| 2019 | CA | SER | B | 375 | 55.660 | 74.967 | 40.213 | 1.00 | 45.63 |
| 2020 | CB | SER | B | 375 | 56.800 | 74.168 | 39.565 | 1.00 | 47.59 |
| 2021 | OG | SER | B | 375 | 56.662 | 74.088 | 38.157 | 1.00 | 49.42 |
| 2022 | C | SER | B | 375 | 55.488 | 76.292 | 39.482 | 1.00 | 45.23 |
| 2023 | O | SER | B | 375 | 54.609 | 76.438 | 38.629 | 1.00 | 46.27 |
| 2024 | N | ARG | B | 376 | 56.323 | 77.259 | 39.843 | 1.00 | 42.63 |
| 2025 | CA | ARG | B | 376 | 56.293 | 78.567 | 39.218 | 1.00 | 41.96 |
| 2026 | CB | ARG | B | 376 | 56.337 | 79.676 | 40.266 | 1.00 | 43.72 |
| 2027 | CG | ARG | B | 376 | 55.142 | 79.745 | 41.199 | 1.00 | 40.62 |
| 2028 | CD | ARG | B | 376 | 54.907 | 81.195 | 41.580 | 1.00 | 41.72 |
| 2029 | NE | ARG | B | 376 | 55.132 | 81.457 | 42.990 | 1.00 | 52.53 |
| 2030 | CZ | ARG | B | 376 | 55.295 | 82.671 | 43.501 | 1.00 | 52.81 |
| 2031 | NH1 | ARG | B | 376 | 55.265 | 83.741 | 42.714 | 1.00 | 51.46 |
| 2032 | NH2 | ARG | B | 376 | 55.472 | 82.819 | 44.807 | 1.00 | 56.61 |
| 2033 | C | ARG | B | 376 | 57.518 | 78.680 | 38.325 | 1.00 | 39.96 |
| 2034 | O | ARG | B | 376 | 58.629 | 78.382 | 38.745 | 1.00 | 40.84 |
| 2035 | N | ALA | B | 377 | 57.315 | 79.097 | 37.086 | 1.00 | 41.64 |
| 2036 | CA | ALA | B | 377 | 58.423 | 79.250 | 36.154 | 1.00 | 41.34 |
| 2037 | CB | ALA | B | 377 | 57.920 | 79.895 | 34.873 | 1.00 | 33.50 |
| 2038 | C | ALA | B | 377 | 59.544 | 80.098 | 36.775 | 1.00 | 41.22 |
| 2039 | O | ALA | B | 377 | 60.712 | 79.915 | 36.452 | 1.00 | 41.04 |
| 2040 | N | SER | B | 378 | 59.179 | 81.008 | 37.677 | 1.00 | 40.92 |
| 2041 | CA | SER | B | 378 | 60.149 | 81.881 | 38.327 | 1.00 | 42.71 |
| 2042 | CB | SER | B | 378 | 59.461 | 83.109 | 38.915 | 1.00 | 44.29 |
| 2043 | OG | SER | B | 378 | 58.733 | 82.758 | 40.080 | 1.00 | 42.78 |
| 2044 | C | SER | B | 378 | 60.894 | 81.180 | 39.442 | 1.00 | 43.58 |
| 2045 | O | SER | B | 378 | 61.809 | 81.749 | 40.022 | 1.00 | 44.22 |
| 2046 | N | GLY | B | 379 | 60.483 | 79.954 | 39.751 | 1.00 | 43.82 |
| 2047 | CA | GLY | B | 379 | 61.137 | 79.191 | 40.796 | 1.00 | 45.23 |
| 2048 | C | GLY | B | 379 | 60.683 | 79.540 | 42.199 | 1.00 | 48.43 |
| 2049 | O | GLY | B | 379 | 61.038 | 78.853 | 43.157 | 1.00 | 47.67 |
| 2050 | N | LYS | B | 380 | 59.901 | 80.605 | 42.337 | 1.00 | 50.04 |
| 2051 | CA | LYS | B | 380 | 59.423 | 80.997 | 43.653 | 1.00 | 52.22 |
| 2052 | CB | LYS | B | 380 | 58.732 | 82.362 | 43.591 | 1.00 | 54.82 |
| 2053 | CG | LYS | B | 380 | 59.721 | 83.504 | 43.454 | 1.00 | 63.15 |
| 2054 | CD | LYS | B | 380 | 59.072 | 84.866 | 43.634 | 1.00 | 70.67 |
| 2055 | CE | LYS | B | 380 | 60.133 | 85.960 | 43.645 | 1.00 | 75.50 |
| 2056 | NZ | LYS | B | 380 | 59.549 | 87.325 | 43.746 | 1.00 | 80.58 |
| 2057 | C | LYS | B | 380 | 58.493 | 79.945 | 44.249 | 1.00 | 52.26 |
| 2058 | O | LYS | B | 380 | 57.944 | 79.105 | 43.535 | 1.00 | 49.96 |
| 2059 | N | PRO | B | 381 | 58.315 | 79.974 | 45.578 | 1.00 | 53.55 |
| 2060 | CD | PRO | B | 381 | 58.943 | 80.906 | 46.529 | 1.00 | 54.43 |
| 2061 | CA | PRO | B | 381 | 57.453 | 79.016 | 46.282 | 1.00 | 54.97 |
| 2062 | CB | PRO | B | 381 | 57.652 | 79.388 | 47.755 | 1.00 | 57.06 |
| 2063 | CG | PRO | B | 381 | 58.989 | 80.081 | 47.773 | 1.00 | 59.10 |
| 2064 | C | PRO | B | 381 | 55.974 | 79.068 | 45.893 | 1.00 | 54.14 |
| 2065 | O | PRO | B | 381 | 55.422 | 80.143 | 45.633 | 1.00 | 55.04 |
| 2066 | N | VAL | B | 382 | 55.344 | 77.899 | 45.849 | 1.00 | 52.78 |
| 2067 | CA | VAL | B | 382 | 53.920 | 77.799 | 45.554 | 1.00 | 53.97 |
| 2068 | CB | VAL | B | 382 | 53.603 | 76.709 | 44.489 | 1.00 | 54.50 |
| 2069 | CG1 | VAL | B | 382 | 54.348 | 77.005 | 43.204 | 1.00 | 56.74 |
| 2070 | CG2 | VAL | B | 382 | 53.955 | 75.319 | 45.022 | 1.00 | 46.72 |
| 2071 | C | VAL | B | 382 | 53.262 | 77.398 | 46.874 | 1.00 | 54.10 |
| 2072 | O | VAL | B | 382 | 53.865 | 76.690 | 47.683 | 1.00 | 53.38 |
| 2073 | N | ASN | B | 383 | 52.034 | 77.848 | 47.089 | 1.00 | 53.55 |
| 2074 | CA | ASN | B | 383 | 51.311 | 77.527 | 48.310 | 1.00 | 54.35 |
| 2075 | CB | ASN | B | 383 | 50.115 | 78.465 | 48.470 | 1.00 | 60.43 |
| 2076 | CG | ASN | B | 383 | 50.513 | 79.923 | 48.471 | 1.00 | 67.43 |
| 2077 | OD1 | ASN | B | 383 | 49.710 | 80.794 | 48.137 | 1.00 | 75.26 |
| 2078 | ND2 | ASN | B | 383 | 51.755 | 80.201 | 48.856 | 1.00 | 72.99 |
| 2079 | C | ASN | B | 383 | 50.821 | 76.080 | 48.318 | 1.00 | 52.64 |
| 2080 | O | ASN | B | 383 | 51.082 | 75.308 | 47.393 | 1.00 | 50.26 |
| 2081 | N | HIS | B | 384 | 50.107 | 75.721 | 49.376 | 1.00 | 51.74 |
| 2082 | CA | HIS | B | 384 | 49.562 | 74.375 | 49.515 | 1.00 | 51.28 |
| 2083 | CB | HIS | B | 384 | 49.220 | 74.101 | 50.979 | 1.00 | 59.06 |
| 2084 | CG | HIS | B | 384 | 50.421 | 73.943 | 51.861 | 1.00 | 65.42 |
| 2085 | CD2 | HIS | B | 384 | 50.873 | 74.688 | 52.896 | 1.00 | 72.26 |
| 2086 | ND1 | HIS | B | 384 | 51.316 | 72.906 | 51.715 | 1.00 | 70.87 |
| 2087 | CE1 | HIS | B | 384 | 52.270 | 73.019 | 52.623 | 1.00 | 76.58 |
| 2088 | NE2 | HIS | B | 384 | 52.024 | 74.093 | 53.353 | 1.00 | 77.95 |
| 2089 | C | HIS | B | 384 | 48.306 | 74.238 | 48.655 | 1.00 | 47.99 |
| 2090 | O | HIS | B | 384 | 47.455 | 75.122 | 48.644 | 1.00 | 45.37 |
| 2091 | N | SER | B | 385 | 48.187 | 73.127 | 47.944 | 1.00 | 45.57 |
| 2092 | CA | SER | B | 385 | 47.036 | 72.926 | 47.085 | 1.00 | 45.69 |
| 2093 | CB | SER | B | 385 | 47.503 | 72.466 | 45.695 | 1.00 | 41.19 |
| 2094 | OG | SER | B | 385 | 48.315 | 71.301 | 45.762 | 1.00 | 41.34 |
| 2095 | C | SER | B | 385 | 46.002 | 71.946 | 47.660 | 1.00 | 44.94 |
| 2096 | O | SER | B | 385 | 46.287 | 71.187 | 48.582 | 1.00 | 41.64 |
| 2097 | N | THR | B | 386 | 44.795 | 71.997 | 47.107 | 1.00 | 45.73 |
| 2098 | CA | THR | B | 386 | 43.702 | 71.133 | 47.521 | 1.00 | 46.08 |
| 2099 | CB | THR | B | 386 | 42.409 | 71.930 | 47.737 | 1.00 | 47.42 |
| 2100 | OG1 | THR | B | 386 | 42.576 | 72.825 | 48.844 | 1.00 | 50.45 |
| 2101 | CG2 | THR | B | 386 | 41.253 | 70.985 | 48.025 | 1.00 | 51.73 |
| 2102 | C | THR | B | 386 | 43.468 | 70.113 | 46.419 | 1.00 | 46.45 |
| 2103 | O | THR | B | 386 | 43.359 | 70.471 | 45.247 | 1.00 | 47.06 |
| 2104 | N | ARG | B | 387 | 43.394 | 68.846 | 46.808 | 1.00 | 46.33 |
| 2105 | CA | ARG | B | 387 | 43.190 | 67.751 | 45.872 | 1.00 | 46.50 |
| 2106 | CB | ARG | B | 387 | 44.310 | 66.726 | 46.070 | 1.00 | 48.08 |
| 2107 | CG | ARG | B | 387 | 44.234 | 65.514 | 45.167 | 1.00 | 54.89 |
| 2108 | CD | ARG | B | 387 | 45.119 | 64.392 | 45.695 | 1.00 | 59.67 |
| 2109 | NE | ARG | B | 387 | 44.932 | 63.162 | 44.935 | 1.00 | 63.12 |
| 2110 | CZ | ARG | B | 387 | 45.650 | 62.825 | 43.870 | 1.00 | 65.65 |
| 2111 | NH1 | ARG | B | 387 | 46.618 | 63.629 | 43.449 | 1.00 | 67.29 |
| 2112 | NH2 | ARG | B | 387 | 45.385 | 61.701 | 43.211 | 1.00 | 63.40 |
| 2113 | C | ARG | B | 387 | 41.814 | 67.083 | 46.054 | 1.00 | 46.30 |
| 2114 | O | ARG | B | 387 | 41.361 | 66.867 | 47.179 | 1.00 | 44.30 |
| 2115 | N | LYS | B | 388 | 41.159 | 66.766 | 44.938 | 1.00 | 45.70 |
| 2116 | CA | LYS | B | 388 | 39.851 | 66.111 | 44.960 | 1.00 | 44.68 |
| 2117 | CB | LYS | B | 388 | 38.748 | 67.121 | 44.634 | 1.00 | 46.84 |
| 2118 | CG | LYS | B | 388 | 38.669 | 68.263 | 45.624 | 1.00 | 56.94 |
| 2119 | CD | LYS | B | 388 | 37.921 | 69.450 | 45.046 | 1.00 | 58.62 |
| 2120 | CE | LYS | B | 388 | 38.293 | 70.717 | 45.791 | 1.00 | 61.92 |
| 2121 | NZ | LYS | B | 388 | 37.744 | 71.924 | 45.123 | 1.00 | 70.07 |
| 2122 | C | LYS | B | 388 | 39.802 | 64.954 | 43.957 | 1.00 | 44.52 |
| 2123 | O | LYS | B | 388 | 40.221 | 65.098 | 42.806 | 1.00 | 40.60 |
| 2124 | N | GLU | B | 389 | 39.296 | 63.807 | 44.411 | 1.00 | 47.26 |
| 2125 | CA | GLU | B | 389 | 39.176 | 62.599 | 43.582 | 1.00 | 50.72 |
| 2126 | CB | GLU | B | 389 | 40.069 | 61.486 | 44.135 | 1.00 | 54.88 |
| 2127 | CG | GLU | B | 389 | 41.559 | 61.752 | 44.061 | 1.00 | 67.37 |
| 2128 | CD | GLU | B | 389 | 42.375 | 60.621 | 44.664 | 1.00 | 75.83 |
| 2129 | OE1 | GLU | B | 389 | 41.858 | 59.484 | 44.736 | 1.00 | 79.33 |
| 2130 | OE2 | GLU | B | 389 | 43.538 | 60.862 | 45.056 | 1.00 | 80.70 |
| 2131 | C | GLU | B | 389 | 37.729 | 62.090 | 43.549 | 1.00 | 51.11 |
| 2132 | O | GLU | B | 389 | 37.090 | 61.956 | 44.596 | 1.00 | 48.28 |
| 2133 | N | ALA | B | 390 | 37.218 | 61.786 | 42.359 | 1.00 | 52.07 |
| 2134 | CA | ALA | B | 390 | 35.845 | 61.303 | 42.232 | 1.00 | 56.84 |
| 2135 | CB | ALA | B | 390 | 34.888 | 62.480 | 42.128 | 1.00 | 53.86 |
| 2136 | C | ALA | B | 390 | 35.643 | 60.371 | 41.042 | 1.00 | 60.96 |
| 2137 | O | ALA | B | 390 | 36.285 | 60.515 | 40.002 | 1.00 | 61.14 |
| 2138 | N | ALA | B | 391 | 34.727 | 59.421 | 41.204 | 1.00 | 65.61 |
| 2139 | CA | ALA | B | 391 | 34.413 | 58.449 | 40.162 | 1.00 | 68.46 |
| 2140 | CB | ALA | B | 391 | 35.264 | 57.202 | 40.339 | 1.00 | 71.73 |
| 2141 | C | ALA | B | 391 | 32.939 | 58.082 | 40.234 | 1.00 | 70.37 |
| 2142 | O | ALA | B | 391 | 32.139 | 58.524 | 39.412 | 1.00 | 72.17 |
| 2143 | N | LEU | B | 397 | 37.390 | 58.285 | 36.020 | 1.00 | 60.70 |
| 2144 | CA | LEU | B | 397 | 37.949 | 58.914 | 37.210 | 1.00 | 61.36 |
| 2145 | CB | LEU | B | 397 | 39.142 | 58.111 | 37.725 | 1.00 | 64.28 |
| 2146 | CG | LEU | B | 397 | 39.864 | 58.746 | 38.920 | 1.00 | 65.19 |
| 2147 | CD1 | LEU | B | 397 | 38.930 | 58.798 | 40.119 | 1.00 | 65.40 |
| 2148 | CD2 | LEU | B | 397 | 41.115 | 57.948 | 39.256 | 1.00 | 68.57 |
| 2149 | C | LEU | B | 397 | 38.389 | 60.359 | 36.981 | 1.00 | 61.24 |
| 2150 | O | LEU | B | 397 | 39.115 | 60.666 | 36.036 | 1.00 | 62.11 |
| 2151 | N | THR | B | 398 | 37.946 | 61.240 | 37.867 | 1.00 | 59.83 |
| 2152 | CA | THR | B | 398 | 38.291 | 62.650 | 37.795 | 1.00 | 58.04 |
| 2153 | CB | THR | B | 398 | 37.028 | 63.539 | 37.837 | 1.00 | 58.72 |
| 2154 | OG1 | THR | B | 398 | 36.322 | 63.429 | 36.594 | 1.00 | 64.28 |
| 2155 | CG2 | THR | B | 398 | 37.403 | 64.992 | 38.077 | 1.00 | 61.55 |
| 2156 | C | THR | B | 398 | 39.199 | 63.027 | 38.965 | 1.00 | 55.72 |
| 2157 | O | THR | B | 398 | 39.005 | 62.579 | 40.100 | 1.00 | 55.02 |
| 2158 | N | VAL | B | 399 | 40.199 | 63.848 | 38.678 | 1.00 | 52.38 |
| 2159 | CA | VAL | B | 399 | 41.127 | 64.303 | 39.698 | 1.00 | 49.13 |
| 2160 | CB | VAL | B | 399 | 42.421 | 63.457 | 39.709 | 1.00 | 51.57 |
| 2161 | CG1 | VAL | B | 399 | 43.388 | 64.006 | 40.737 | 1.00 | 52.00 |
| 2162 | CG2 | VAL | B | 399 | 42.100 | 62.010 | 40.032 | 1.00 | 55.81 |
| 2163 | C | VAL | B | 399 | 41.507 | 65.753 | 39.457 | 1.00 | 46.27 |
| 2164 | O | VAL | B | 399 | 41.951 | 66.122 | 38.368 | 1.00 | 43.17 |
| 2165 | N | THR | B | 400 | 41.311 | 66.585 | 40.470 | 1.00 | 44.56 |
| 2166 | CA | THR | B | 400 | 41.685 | 67.981 | 40.342 | 1.00 | 43.39 |
| 2167 | CB | THR | B | 400 | 40.460 | 68.931 | 40.271 | 1.00 | 41.43 |
| 2168 | OG1 | THR | B | 400 | 39.806 | 68.972 | 41.543 | 1.00 | 48.65 |
| 2169 | CG2 | THR | B | 400 | 39.480 | 68.474 | 39.211 | 1.00 | 38.93 |
| 2170 | C | THR | B | 400 | 42.541 | 68.426 | 41.516 | 1.00 | 40.85 |
| 2171 | O | THR | B | 400 | 42.506 | 67.849 | 42.606 | 1.00 | 35.85 |
| 2172 | N | SER | B | 401 | 43.333 | 69.454 | 41.253 | 1.00 | 39.13 |
| 2173 | CA | SER | B | 401 | 44.183 | 70.058 | 42.253 | 1.00 | 37.93 |
| 2174 | CB | SER | B | 401 | 45.656 | 69.715 | 42.021 | 1.00 | 40.15 |
| 2175 | OG | SER | B | 401 | 46.458 | 70.310 | 43.031 | 1.00 | 44.51 |
| 2176 | C | SER | B | 401 | 43.978 | 71.546 | 42.056 | 1.00 | 36.16 |
| 2177 | O | SER | B | 401 | 44.139 | 72.048 | 40.947 | 1.00 | 36.19 |
| 2178 | N | THR | B | 402 | 43.611 | 72.238 | 43.125 | 1.00 | 32.94 |
| 2179 | CA | THR | B | 402 | 43.396 | 73.669 | 43.061 | 1.00 | 32.35 |
| 2180 | CB | THR | B | 402 | 42.022 | 74.022 | 43.617 | 1.00 | 33.13 |
| 2181 | OG1 | THR | B | 402 | 41.041 | 73.321 | 42.854 | 1.00 | 34.46 |
| 2182 | CG2 | THR | B | 402 | 41.755 | 75.514 | 43.523 | 1.00 | 33.71 |
| 2183 | C | THR | B | 402 | 44.483 | 74.378 | 43.857 | 1.00 | 31.66 |
| 2184 | O | THR | B | 402 | 44.714 | 74.088 | 45.036 | 1.00 | 26.99 |
| 2185 | N | LEU | B | 403 | 45.141 | 75.318 | 43.187 | 1.00 | 32.16 |
| 2186 | CA | LEU | B | 403 | 46.230 | 76.074 | 43.781 | 1.00 | 32.46 |
| 2187 | CB | LEU | B | 403 | 47.477 | 75.952 | 42.895 | 1.00 | 34.12 |
| 2188 | CG | LEU | B | 403 | 48.762 | 76.680 | 43.306 | 1.00 | 34.78 |
| 2189 | CD1 | LEU | B | 403 | 49.404 | 75.922 | 44.456 | 1.00 | 35.87 |
| 2190 | CD2 | LEU | B | 403 | 49.727 | 76.744 | 42.132 | 1.00 | 31.42 |
| 2191 | C | LEU | B | 403 | 45.910 | 77.548 | 44.001 | 1.00 | 33.39 |
| 2192 | O | LEU | B | 403 | 45.595 | 78.279 | 43.060 | 1.00 | 30.14 |
| 2193 | N | PRO | B | 404 | 45.967 | 77.996 | 45.265 | 1.00 | 34.13 |
| 2194 | CD | PRO | B | 404 | 46.187 | 77.209 | 46.495 | 1.00 | 34.29 |
| 2195 | CA | PRO | B | 404 | 45.701 | 79.397 | 45.587 | 1.00 | 34.91 |
| 2196 | CB | PRO | B | 404 | 45.823 | 79.439 | 47.108 | 1.00 | 33.92 |
| 2197 | CG | PRO | B | 404 | 45.479 | 78.046 | 47.526 | 1.00 | 33.77 |
| 2198 | C | PRO | B | 404 | 46.827 | 80.173 | 44.909 | 1.00 | 36.43 |
| 2199 | O | PRO | B | 404 | 47.990 | 79.776 | 44.960 | 1.00 | 30.87 |
| 2200 | N | VAL | B | 405 | 46.480 | 81.274 | 44.265 | 1.00 | 37.52 |
| 2201 | CA | VAL | B | 405 | 47.471 | 82.068 | 43.569 | 1.00 | 41.37 |
| 2202 | CB | VAL | B | 405 | 47.138 | 82.078 | 42.047 | 1.00 | 44.39 |
| 2203 | CG1 | VAL | B | 405 | 47.034 | 83.500 | 41.513 | 1.00 | 47.81 |
| 2204 | CG2 | VAL | B | 405 | 48.180 | 81.288 | 41.301 | 1.00 | 43.63 |
| 2205 | C | VAL | B | 405 | 47.544 | 83.492 | 44.120 | 1.00 | 42.93 |
| 2206 | O | VAL | B | 405 | 46.553 | 84.042 | 44.589 | 1.00 | 42.10 |
| 2207 | N | GLY | B | 406 | 48.732 | 84.082 | 44.081 | 1.00 | 45.32 |
| 2208 | CA | GLY | B | 406 | 48.869 | 85.446 | 44.553 | 1.00 | 46.40 |
| 2209 | C | GLY | B | 406 | 48.208 | 86.373 | 43.548 | 1.00 | 46.70 |
| 2210 | O | GLY | B | 406 | 48.345 | 86.179 | 42.334 | 1.00 | 41.89 |
| 2211 | N | THR | B | 407 | 47.486 | 87.373 | 44.052 | 1.00 | 46.37 |
| 2212 | CA | THR | B | 407 | 46.791 | 88.341 | 43.208 | 1.00 | 49.05 |
| 2213 | CB | THR | B | 407 | 45.983 | 89.348 | 44.062 | 1.00 | 51.01 |
| 2214 | OG1 | THR | B | 407 | 44.900 | 88.671 | 44.709 | 1.00 | 55.76 |
| 2215 | CG2 | THR | B | 407 | 45.423 | 90.453 | 43.195 | 1.00 | 54.66 |
| 2216 | C | THR | B | 407 | 47.746 | 89.132 | 42.325 | 1.00 | 49.24 |
| 2217 | O | THR | B | 407 | 47.597 | 89.154 | 41.105 | 1.00 | 48.91 |
| 2218 | N | ALA | B | 408 | 48.726 | 89.777 | 42.953 | 1.00 | 49.71 |
| 2219 | CA | ALA | B | 408 | 49.693 | 90.592 | 42.240 | 1.00 | 50.13 |
| 2220 | CB | ALA | B | 408 | 50.562 | 91.367 | 43.224 | 1.00 | 53.74 |
| 2221 | C | ALA | B | 408 | 50.569 | 89.777 | 41.312 | 1.00 | 51.25 |
| 2222 | O | ALA | B | 408 | 50.852 | 90.207 | 40.191 | 1.00 | 50.28 |
| 2223 | N | ASP | B | 409 | 51.000 | 88.605 | 41.770 | 1.00 | 50.74 |
| 2224 | CA | ASP | B | 409 | 51.852 | 87.748 | 40.948 | 1.00 | 52.13 |
| 2225 | CB | ASP | B | 409 | 52.265 | 86.498 | 41.730 | 1.00 | 58.27 |
| 2226 | CG | ASP | B | 409 | 53.065 | 86.830 | 42.977 | 1.00 | 69.38 |
| 2227 | OD1 | ASP | B | 409 | 54.121 | 87.495 | 42.848 | 1.00 | 75.94 |
| 2228 | OD2 | ASP | B | 409 | 52.641 | 86.431 | 44.084 | 1.00 | 72.00 |
| 2229 | C | ASP | B | 409 | 51.151 | 87.341 | 39.655 | 1.00 | 49.96 |
| 2230 | O | ASP | B | 409 | 51.769 | 87.283 | 38.588 | 1.00 | 49.48 |
| 2231 | N | TRP | B | 410 | 49.858 | 87.058 | 39.755 | 1.00 | 46.74 |
| 2232 | CA | TRP | B | 410 | 49.106 | 86.669 | 38.583 | 1.00 | 44.75 |
| 2233 | CB | TRP | B | 410 | 47.732 | 86.113 | 38.957 | 1.00 | 39.75 |
| 2234 | CG | TRP | B | 410 | 46.999 | 85.687 | 37.738 | 1.00 | 37.35 |
| 2235 | CD2 | TRP | B | 410 | 47.191 | 84.469 | 37.017 | 1.00 | 31.03 |
| 2236 | CE2 | TRP | B | 410 | 46.412 | 84.551 | 35.846 | 1.00 | 29.68 |
| 2237 | CE3 | TRP | B | 410 | 47.963 | 83.314 | 37.243 | 1.00 | 32.46 |
| 2238 | CD1 | TRP | B | 410 | 46.119 | 86.434 | 37.001 | 1.00 | 32.61 |
| 2239 | NE1 | TRP | B | 410 | 45.767 | 85.758 | 35.868 | 1.00 | 33.45 |
| 2240 | CZ2 | TRP | B | 410 | 46.370 | 83.522 | 34.900 | 1.00 | 29.11 |
| 2241 | CZ3 | TRP | B | 410 | 47.920 | 82.290 | 36.302 | 1.00 | 34.12 |
| 2242 | CH2 | TRP | B | 410 | 47.128 | 82.405 | 35.142 | 1.00 | 35.80 |
| 2243 | C | TRP | B | 410 | 48.934 | 87.857 | 37.649 | 1.00 | 43.08 |
| 2244 | O | TRP | B | 410 | 49.137 | 87.733 | 36.445 | 1.00 | 38.95 |
| 2245 | N | ILE | B | 411 | 48.563 | 89.007 | 38.206 | 1.00 | 46.39 |
| 2246 | CA | ILE | B | 411 | 48.372 | 90.211 | 37.404 | 1.00 | 51.22 |
| 2247 | CB | ILE | B | 411 | 47.848 | 91.382 | 38.259 | 1.00 | 53.02 |
| 2248 | CG2 | ILE | B | 411 | 47.705 | 92.628 | 37.401 | 1.00 | 55.27 |
| 2249 | CG1 | ILE | B | 411 | 46.488 | 91.010 | 38.860 | 1.00 | 56.63 |
| 2250 | CD1 | ILE | B | 411 | 45.858 | 92.102 | 39.733 | 1.00 | 61.16 |
| 2251 | C | ILE | B | 411 | 49.669 | 90.632 | 36.729 | 1.00 | 52.33 |
| 2252 | O | ILE | B | 411 | 49.645 | 91.196 | 35.638 | 1.00 | 53.32 |
| 2253 | N | GLU | B | 412 | 50.800 | 90.333 | 37.369 | 1.00 | 54.18 |
| 2254 | CA | GLU | B | 412 | 52.099 | 90.693 | 36.821 | 1.00 | 53.56 |
| 2255 | CB | GLU | B | 412 | 53.110 | 90.908 | 37.942 | 1.00 | 58.95 |
| 2256 | CG | GLU | B | 412 | 53.034 | 92.302 | 38.528 | 1.00 | 73.61 |
| 2257 | CD | GLU | B | 412 | 54.062 | 92.542 | 39.612 | 1.00 | 83.01 |
| 2258 | OE1 | GLU | B | 412 | 55.244 | 92.185 | 39.411 | 1.00 | 89.18 |
| 2259 | OE2 | GLU | B | 412 | 53.691 | 93.102 | 40.665 | 1.00 | 89.24 |
| 2260 | C | GLU | B | 412 | 52.665 | 89.726 | 35.793 | 1.00 | 51.42 |
| 2261 | O | GLU | B | 412 | 53.725 | 89.985 | 35.227 | 1.00 | 50.68 |
| 2262 | N | GLY | B | 413 | 51.967 | 88.617 | 35.551 | 1.00 | 48.27 |
| 2263 | CA | GLY | B | 413 | 52.428 | 87.671 | 34.551 | 1.00 | 46.15 |
| 2264 | C | GLY | B | 413 | 53.111 | 86.377 | 34.967 | 1.00 | 44.58 |
| 2265 | O | GLY | B | 413 | 53.637 | 85.675 | 34.106 | 1.00 | 42.91 |
| 2266 | N | GLU | B | 414 | 53.118 | 86.044 | 36.256 | 1.00 | 44.06 |
| 2267 | CA | GLU | B | 414 | 53.749 | 84.799 | 36.685 | 1.00 | 42.21 |
| 2268 | CB | GLU | B | 414 | 53.467 | 84.523 | 38.172 | 1.00 | 42.27 |
| 2269 | CG | GLU | B | 414 | 53.963 | 83.159 | 38.698 | 1.00 | 44.30 |
| 2270 | CD | GLU | B | 414 | 55.487 | 83.036 | 38.739 | 1.00 | 52.18 |
| 2271 | OE1 | GLU | B | 414 | 56.083 | 82.562 | 37.745 | 1.00 | 51.39 |
| 2272 | OE2 | GLU | B | 414 | 56.090 | 83.425 | 39.764 | 1.00 | 49.53 |
| 2273 | C | GLU | B | 414 | 53.147 | 83.690 | 35.839 | 1.00 | 41.40 |
| 2274 | O | GLU | B | 414 | 51.995 | 83.781 | 35.413 | 1.00 | 38.41 |
| 2275 | N | THR | B | 415 | 53.936 | 82.656 | 35.573 | 1.00 | 41.90 |
| 2276 | CA | THR | B | 415 | 53.454 | 81.522 | 34.806 | 1.00 | 43.26 |
| 2277 | CB | THR | B | 415 | 54.281 | 81.341 | 33.506 | 1.00 | 46.09 |
| 2278 | OG1 | THR | B | 415 | 54.155 | 79.996 | 33.032 | 1.00 | 50.78 |
| 2279 | CG2 | THR | B | 415 | 55.716 | 81.690 | 33.734 | 1.00 | 56.55 |
| 2280 | C | THR | B | 415 | 53.499 | 80.270 | 35.690 | 1.00 | 41.87 |
| 2281 | O | THR | B | 415 | 54.533 | 79.963 | 36.285 | 1.00 | 43.61 |
| 2282 | N | TYR | B | 416 | 52.371 | 79.571 | 35.806 | 1.00 | 39.27 |
| 2283 | CA | TYR | B | 416 | 52.309 | 78.378 | 36.650 | 1.00 | 37.37 |
| 2284 | CB | TYR | B | 416 | 51.102 | 78.428 | 37.581 | 1.00 | 33.61 |
| 2285 | CG | TYR | B | 416 | 51.064 | 79.633 | 38.478 | 1.00 | 27.57 |
| 2286 | CD1 | TYR | B | 416 | 50.753 | 80.894 | 37.974 | 1.00 | 26.31 |
| 2287 | CE1 | TYR | B | 416 | 50.752 | 82.009 | 38.800 | 1.00 | 35.08 |
| 2288 | CD2 | TYR | B | 416 | 51.371 | 79.516 | 39.825 | 1.00 | 28.68 |
| 2289 | CE2 | TYR | B | 416 | 51.379 | 80.615 | 40.651 | 1.00 | 30.56 |
| 2290 | CZ | TYR | B | 416 | 51.070 | 81.857 | 40.140 | 1.00 | 35.62 |
| 2291 | OH | TYR | B | 416 | 51.088 | 82.944 | 40.984 | 1.00 | 44.94 |
| 2292 | C | TYR | B | 416 | 52.256 | 77.106 | 35.833 | 1.00 | 38.31 |
| 2293 | O | TYR | B | 416 | 51.744 | 77.092 | 34.709 | 1.00 | 37.29 |
| 2294 | N | GLN | B | 417 | 52.781 | 76.031 | 36.416 | 1.00 | 39.74 |
| 2295 | CA | GLN | B | 417 | 52.823 | 74.751 | 35.736 | 1.00 | 40.75 |
| 2296 | CB | GLN | B | 417 | 54.254 | 74.457 | 35.264 | 1.00 | 41.60 |
| 2297 | CG | GLN | B | 417 | 54.353 | 73.240 | 34.349 | 1.00 | 53.57 |
| 2298 | CD | GLN | B | 417 | 55.762 | 72.997 | 33.829 | 1.00 | 65.04 |
| 2299 | OE1 | GLN | B | 417 | 56.481 | 72.124 | 34.325 | 1.00 | 69.74 |
| 2300 | NE2 | GLN | B | 417 | 56.165 | 73.777 | 32.831 | 1.00 | 66.91 |
| 2301 | C | GLN | B | 417 | 52.320 | 73.578 | 36.565 | 1.00 | 39.48 |
| 2302 | O | GLN | B | 417 | 52.619 | 73.451 | 37.754 | 1.00 | 40.03 |
| 2303 | N | CYS | B | 418 | 51.557 | 72.718 | 35.912 | 1.00 | 39.47 |
| 2304 | CA | CYS | B | 418 | 51.032 | 71.525 | 36.541 | 1.00 | 41.39 |
| 2305 | C | CYS | B | 418 | 51.732 | 70.354 | 35.868 | 1.00 | 41.00 |
| 2306 | O | CYS | B | 418 | 51.661 | 70.205 | 34.648 | 1.00 | 41.94 |
| 2307 | CB | CYS | B | 418 | 49.513 | 71.413 | 36.319 | 1.00 | 39.12 |
| 2308 | SG | CYS | B | 418 | 48.782 | 69.912 | 37.050 | 1.00 | 53.87 |
| 2309 | N | ALA | B | 419 | 52.429 | 69.541 | 36.654 | 1.00 | 42.75 |
| 2310 | CA | ALA | B | 419 | 53.102 | 68.362 | 36.118 | 1.00 | 45.21 |
| 2311 | CB | ALA | B | 419 | 54.527 | 68.243 | 36.688 | 1.00 | 45.00 |
| 2312 | C | ALA | B | 419 | 52.265 | 67.138 | 36.498 | 1.00 | 46.28 |
| 2313 | O | ALA | B | 419 | 52.172 | 66.774 | 37.671 | 1.00 | 44.60 |
| 2314 | N | VAL | B | 420 | 51.639 | 66.512 | 35.508 | 1.00 | 49.65 |
| 2315 | CA | VAL | B | 420 | 50.811 | 65.348 | 35.778 | 1.00 | 54.91 |
| 2316 | CB | VAL | B | 420 | 49.584 | 65.305 | 34.855 | 1.00 | 52.57 |
| 2317 | CG1 | VAL | B | 420 | 48.742 | 64.085 | 35.189 | 1.00 | 54.41 |
| 2318 | CG2 | VAL | B | 420 | 48.756 | 66.568 | 35.017 | 1.00 | 52.89 |
| 2319 | C | VAL | B | 420 | 51.572 | 64.036 | 35.623 | 1.00 | 59.75 |
| 2320 | O | VAL | B | 420 | 52.083 | 63.723 | 34.544 | 1.00 | 56.32 |
| 2321 | N | THR | B | 421 | 51.640 | 63.275 | 36.713 | 1.00 | 65.72 |
| 2322 | CA | THR | B | 421 | 52.322 | 61.985 | 36.717 | 1.00 | 72.95 |
| 2323 | CB | THR | B | 421 | 53.303 | 61.867 | 37.903 | 1.00 | 73.73 |
| 2324 | OG1 | THR | B | 421 | 54.294 | 62.898 | 37.818 | 1.00 | 78.26 |
| 2325 | CG2 | THR | B | 421 | 53.995 | 60.518 | 37.883 | 1.00 | 74.84 |
| 2326 | C | THR | B | 421 | 51.302 | 60.859 | 36.831 | 1.00 | 76.77 |
| 2327 | O | THR | B | 421 | 50.937 | 60.459 | 37.936 | 1.00 | 76.76 |
| 2328 | N | ALA | B | 422 | 50.843 | 60.352 | 35.690 | 1.00 | 81.68 |
| 2329 | CA | ALA | B | 422 | 49.862 | 59.270 | 35.673 | 1.00 | 86.81 |
| 2330 | CB | ALA | B | 422 | 49.134 | 59.242 | 34.332 | 1.00 | 86.67 |
| 2331 | C | ALA | B | 422 | 50.540 | 57.930 | 35.922 | 1.00 | 90.55 |
| 2332 | O | ALA | B | 422 | 51.724 | 57.757 | 35.623 | 1.00 | 91.00 |
| 2333 | N | PRO | B | 423 | 49.793 | 56.959 | 36.475 | 1.00 | 93.77 |
| 2334 | CD | PRO | B | 423 | 48.340 | 56.985 | 36.715 | 1.00 | 94.85 |
| 2335 | CA | PRO | B | 423 | 50.343 | 55.630 | 36.759 | 1.00 | 95.83 |
| 2336 | CB | PRO | B | 423 | 49.141 | 54.869 | 37.309 | 1.00 | 96.70 |
| 2337 | CG | PRO | B | 423 | 47.981 | 55.526 | 36.613 | 1.00 | 97.10 |
| 2338 | C | PRO | B | 423 | 50.926 | 54.988 | 35.504 | 1.00 | 97.16 |
| 2339 | O | PRO | B | 423 | 52.128 | 54.750 | 35.426 | 1.00 | 97.34 |
| 2340 | N | ALA | B | 424 | 50.076 | 54.707 | 34.522 | 1.00 | 98.50 |
| 2341 | CA | ALA | B | 424 | 50.537 | 54.114 | 33.271 | 1.00 | 99.73 |
| 2342 | CB | ALA | B | 424 | 49.348 | 53.605 | 32.474 | 1.00 | 99.11 |
| 2343 | C | ALA | B | 424 | 51.282 | 55.188 | 32.475 | 1.00 | 100.69 |
| 2344 | O | ALA | B | 424 | 51.875 | 56.097 | 33.054 | 1.00 | 101.64 |
| 2345 | N | LEU | B | 425 | 51.245 | 55.079 | 31.150 | 1.00 | 100.65 |
| 2346 | CA | LEU | B | 425 | 51.895 | 56.050 | 30.272 | 1.00 | 99.91 |
| 2347 | CB | LEU | B | 425 | 51.228 | 57.418 | 30.429 | 1.00 | 100.27 |
| 2348 | CG | LEU | B | 425 | 49.767 | 57.474 | 29.975 | 1.00 | 102.88 |
| 2349 | CD1 | LEU | B | 425 | 49.214 | 58.865 | 30.211 | 1.00 | 102.68 |
| 2350 | CD2 | LEU | B | 425 | 49.668 | 57.102 | 28.497 | 1.00 | 105.00 |
| 2351 | C | LEU | B | 425 | 53.400 | 56.173 | 30.514 | 1.00 | 98.82 |
| 2352 | O | LEU | B | 425 | 53.924 | 55.615 | 31.471 | 1.00 | 98.87 |
| 2353 | N | PRO | B | 426 | 54.115 | 56.888 | 29.625 | 1.00 | 97.69 |
| 2354 | CD | PRO | B | 426 | 53.661 | 57.182 | 28.251 | 1.00 | 98.02 |
| 2355 | CA | PRO | B | 426 | 55.566 | 57.087 | 29.739 | 1.00 | 95.83 |
| 2356 | CB | PRO | B | 426 | 56.030 | 56.927 | 28.303 | 1.00 | 96.48 |
| 2357 | CG | PRO | B | 426 | 54.941 | 57.635 | 27.555 | 1.00 | 96.97 |
| 2358 | C | PRO | B | 426 | 55.949 | 58.455 | 30.309 | 1.00 | 94.21 |
| 2359 | O | PRO | B | 426 | 56.240 | 58.594 | 31.498 | 1.00 | 94.03 |
| 2360 | N | ARG | B | 427 | 55.963 | 59.460 | 29.439 | 1.00 | 92.33 |
| 2361 | CA | ARG | B | 427 | 56.303 | 60.822 | 29.831 | 1.00 | 90.72 |
| 2362 | CB | ARG | B | 427 | 56.583 | 61.672 | 28.590 | 1.00 | 92.36 |
| 2363 | CG | ARG | B | 427 | 57.784 | 61.251 | 27.758 | 1.00 | 96.36 |
| 2364 | CD | ARG | B | 427 | 59.015 | 62.075 | 28.102 | 1.00 | 99.46 |
| 2365 | NE | ARG | B | 427 | 59.822 | 62.353 | 26.916 | 1.00 | 101.30 |
| 2366 | CZ | ARG | B | 427 | 59.397 | 63.060 | 25.872 | 1.00 | 102.28 |
| 2367 | NH1 | ARG | B | 427 | 58.170 | 63.563 | 25.864 | 1.00 | 102.89 |
| 2368 | NH2 | ARG | B | 427 | 60.200 | 63.266 | 24.836 | 1.00 | 102.96 |
| 2369 | C | AEG | B | 427 | 55.142 | 61.448 | 30.595 | 1.00 | 88.67 |
| 2370 | O | ARG | B | 427 | 53.986 | 61.308 | 30.198 | 1.00 | 88.90 |
| 2371 | N | ALA | B | 428 | 55.447 | 62.138 | 31.687 | 1.00 | 85.79 |
| 2372 | CA | ALA | B | 428 | 54.412 | 62.793 | 32.469 | 1.00 | 82.45 |
| 2373 | CB | ALA | B | 428 | 54.985 | 63.290 | 33.789 | 1.00 | 82.49 |
| 2374 | C | ALA | B | 428 | 53.893 | 63.963 | 31.644 | 1.00 | 79.74 |
| 2375 | O | ALA | B | 428 | 54.645 | 64.568 | 30.879 | 1.00 | 79.87 |
| 2376 | N | LEU | B | 429 | 52.606 | 64.270 | 31.785 | 1.00 | 76.91 |
| 2377 | CA | LEU | B | 429 | 51.996 | 65.377 | 31.051 | 1.00 | 72.60 |
| 2378 | CB | LEU | B | 429 | 50.475 | 65.195 | 30.974 | 1.00 | 73.30 |
| 2379 | CG | LEU | B | 429 | 49.915 | 63.916 | 30.343 | 1.00 | 75.14 |
| 2380 | CD1 | LEU | B | 429 | 48.403 | 63.894 | 30.500 | 1.00 | 70.88 |
| 2381 | CD2 | LEU | B | 429 | 50.304 | 63.844 | 28.873 | 1.00 | 79.00 |
| 2382 | C | LEU | B | 429 | 52.310 | 66.679 | 31.777 | 1.00 | 69.58 |
| 2383 | O | LEU | B | 429 | 52.391 | 66.706 | 33.006 | 1.00 | 67.68 |
| 2384 | N | MET | B | 430 | 52.484 | 67.755 | 31.015 | 1.00 | 66.58 |
| 2385 | CA | MET | B | 430 | 52.785 | 69.057 | 31.596 | 1.00 | 63.72 |
| 2386 | CB | MET | B | 430 | 54.260 | 69.397 | 31.396 | 1.00 | 66.69 |
| 2387 | CG | MET | B | 430 | 55.205 | 68.363 | 31.975 | 1.00 | 76.81 |
| 2388 | SD | MET | B | 430 | 56.932 | 68.836 | 31.755 | 1.00 | 89.36 |
| 2389 | CE | MET | B | 430 | 57.129 | 68.610 | 29.990 | 1.00 | 87.45 |
| 2390 | C | MET | B | 430 | 51.926 | 70.150 | 30.981 | 1.00 | 59.15 |
| 2391 | O | MET | B | 430 | 51.778 | 70.229 | 29.760 | 1.00 | 59.09 |
| 2392 | N | ARG | B | 431 | 51.355 | 70.993 | 31.833 | 1.00 | 55.24 |
| 2393 | CA | AEG | B | 431 | 50.509 | 72.077 | 31.354 | 1.00 | 51.07 |
| 2394 | CB | ARG | B | 431 | 49.036 | 71.737 | 31.584 | 1.00 | 51.17 |
| 2395 | CG | ARG | B | 431 | 48.661 | 70.309 | 31.203 | 1.00 | 55.59 |
| 2396 | CD | ARG | B | 431 | 47.441 | 70.280 | 30.295 | 1.00 | 67.55 |
| 2397 | NE | ARG | B | 431 | 47.784 | 70.453 | 28.884 | 1.00 | 74.62 |
| 2398 | CZ | ARG | B | 431 | 48.220 | 69.472 | 28.099 | 1.00 | 76.90 |
| 2399 | NH1 | ARG | B | 431 | 48.364 | 68.247 | 28.586 | 1.00 | 77.02 |
| 2400 | NH2 | ARG | B | 431 | 48.513 | 69.712 | 26.826 | 1.00 | 79.64 |
| 2401 | C | ARG | B | 431 | 50.868 | 73.356 | 32.087 | 1.00 | 47.90 |
| 2402 | O | ARG | B | 431 | 51.214 | 73.323 | 33.268 | 1.00 | 46.26 |
| 2403 | N | SER | B | 432 | 50.788 | 74.480 | 31.385 | 1.00 | 45.54 |
| 2404 | CA | SER | B | 432 | 51.105 | 75.768 | 31.988 | 1.00 | 45.94 |
| 2405 | CB | SER | B | 432 | 52.484 | 76.244 | 31.516 | 1.00 | 47.12 |
| 2406 | OC | SER | B | 432 | 52.560 | 76.269 | 30.106 | 1.00 | 50.92 |
| 2407 | C | SER | B | 432 | 50.063 | 76.836 | 31.688 | 1.00 | 43.27 |
| 2408 | O | SER | B | 432 | 49.328 | 76.738 | 30.709 | 1.00 | 42.70 |
| 2409 | N | THR | B | 433 | 50.012 | 77.864 | 32.531 | 1.00 | 40.97 |
| 2410 | CA | THR | B | 433 | 49.048 | 78.937 | 32.342 | 1.00 | 38.90 |
| 2411 | CB | THR | B | 433 | 47.725 | 78.633 | 33.073 | 1.00 | 39.50 |
| 2412 | OG1 | THR | B | 433 | 46.792 | 79.689 | 32.820 | 1.00 | 38.91 |
| 2413 | CG2 | THR | B | 433 | 47.954 | 78.507 | 34.584 | 1.00 | 36.51 |
| 2414 | C | THR | B | 433 | 49.583 | 80.271 | 32.838 | 1.00 | 39.62 |
| 2415 | O | THR | B | 433 | 50.412 | 80.317 | 33.744 | 1.00 | 39.87 |
| 2416 | N | THR | B | 434 | 49.105 | 81.351 | 32.224 | 1.00 | 39.10 |
| 2417 | CA | THR | B | 434 | 49.499 | 82.711 | 32.569 | 1.00 | 39.67 |
| 2418 | CB | THR | B | 434 | 50.788 | 83.120 | 31.818 | 1.00 | 43.40 |
| 2419 | OG1 | THR | B | 434 | 50.496 | 83.293 | 30.425 | 1.00 | 53.59 |
| 2420 | CG2 | THR | B | 434 | 51.830 | 82.041 | 31.929 | 1.00 | 52.29 |
| 2421 | C | THR | B | 434 | 48.380 | 83.661 | 32.131 | 1.00 | 38.41 |
| 2422 | O | THR | B | 434 | 47.490 | 83.275 | 31.374 | 1.00 | 38.84 |
| 2423 | N | ALA | B | 435 | 48.421 | 84.899 | 32.594 | 1.00 | 37.73 |
| 2424 | CA | ALA | B | 435 | 47.410 | 85.853 | 32.189 | 1.00 | 44.38 |
| 2425 | CB | ALA | B | 435 | 47.632 | 87.174 | 32.887 | 1.00 | 40.72 |
| 2426 | C | ALA | B | 435 | 47.567 | 86.023 | 30.679 | 1.00 | 47.78 |
| 2427 | O | ALA | B | 435 | 48.681 | 85.989 | 30.163 | 1.00 | 46.98 |
| 2428 | N | TNR | B | 436 | 46.454 | 86.187 | 29.971 | 1.00 | 52.31 |
| 2429 | CA | THR | B | 436 | 46.506 | 86.383 | 28.526 | 1.00 | 55.83 |
| 2430 | CB | THR | B | 436 | 45.131 | 86.108 | 27.860 | 1.00 | 59.83 |
| 2431 | OG1 | THR | B | 436 | 44.784 | 84.727 | 28.028 | 1.00 | 62.64 |
| 2432 | CG2 | THR | B | 436 | 45.175 | 86.441 | 26.368 | 1.00 | 60.56 |
| 2433 | C | THR | B | 436 | 46.893 | 87.834 | 28.260 | 1.00 | 56.91 |
| 2434 | O | THR | B | 436 | 46.358 | 88.748 | 28.888 | 1.00 | 55.51 |
| 2435 | N | SER | B | 437 | 47.841 | 88.043 | 27.352 | 1.00 | 58.87 |
| 2436 | CA | SER | B | 437 | 48.256 | 89.398 | 27.005 | 1.00 | 61.80 |
| 2437 | CB | SER | B | 437 | 49.746 | 89.445 | 26.626 | 1.00 | 63.69 |
| 2438 | OG | SER | B | 437 | 50.029 | 88.616 | 25.511 | 1.00 | 67.24 |
| 2439 | C | SER | B | 437 | 47.397 | 89.847 | 25.828 | 1.00 | 61.35 |
| 2440 | O | SER | B | 437 | 46.489 | 89.123 | 25.410 | 1.00 | 61.92 |
| 2441 | N | GLY | B | 438 | 47.670 | 91.033 | 25.297 | 1.00 | 60.43 |
| 2442 | CA | GLY | B | 438 | 46.881 | 91.513 | 24.177 | 1.00 | 59.27 |
| 2443 | C | GLY | B | 438 | 45.909 | 92.601 | 24.590 | 1.00 | 58.25 |
| 2444 | O | GLY | B | 438 | 45.753 | 92.861 | 25.779 | 1.00 | 58.13 |
| 2445 | N | PRO | B | 439 | 45.237 | 93.253 | 23.628 | 1.00 | 58.84 |
| 2446 | CD | PRO | B | 439 | 45.385 | 93.045 | 22.177 | 1.00 | 60.09 |
| 2447 | CA | PRO | B | 439 | 44.277 | 94.325 | 23.897 | 1.00 | 57.04 |
| 2448 | CB | PRO | B | 439 | 43.768 | 94.692 | 22.503 | 1.00 | 57.59 |
| 2449 | CG | PRO | B | 439 | 44.933 | 94.376 | 21.628 | 1.00 | 59.40 |
| 2450 | C | PRO | B | 439 | 43.147 | 93.909 | 24.828 | 1.00 | 54.66 |
| 2451 | O | PRO | B | 439 | 42.824 | 92.723 | 24.945 | 1.00 | 56.08 |
| 2452 | N | ARG | B | 440 | 42.548 | 94.897 | 25.482 | 1.00 | 51.95 |
| 2453 | CA | ARG | B | 440 | 41.451 | 94.658 | 26.405 | 1.00 | 49.50 |
| 2454 | CB | ARG | B | 440 | 41.829 | 95.129 | 27.817 | 1.00 | 51.89 |
| 2455 | CG | ARG | B | 440 | 43.200 | 94.679 | 28.309 | 1.00 | 59.84 |
| 2456 | CD | ARG | B | 440 | 43.266 | 93.188 | 28.635 | 1.00 | 66.22 |
| 2457 | NE | ARG | B | 440 | 44.657 | 92.755 | 28.787 | 1.00 | 74.61 |
| 2458 | CZ | ARG | B | 440 | 45.045 | 91.553 | 29.204 | 1.00 | 76.10 |
| 2459 | NH1 | ARG | B | 440 | 44.156 | 90.633 | 29.527 | 1.00 | 77.20 |
| 2460 | NH2 | ARG | B | 440 | 46.338 | 91.272 | 29.295 | 1.00 | 84.96 |
| 2461 | C | ARG | B | 440 | 40.249 | 95.455 | 25.910 | 1.00 | 46.80 |
| 2462 | O | ARG | B | 440 | 40.406 | 96.542 | 25.354 | 1.00 | 46.09 |
| 2463 | N | ALA | B | 441 | 39.053 | 94.904 | 26.099 | 1.00 | 42.67 |
| 2464 | CA | ALA | B | 441 | 37.821 | 95.576 | 25.698 | 1.00 | 39.13 |
| 2465 | CB | ALA | B | 441 | 37.458 | 95.216 | 24.263 | 1.00 | 32.25 |
| 2466 | C | ALA | B | 441 | 36.712 | 95.142 | 26.646 | 1.00 | 39.00 |
| 2467 | O | ALA | B | 441 | 36.555 | 93.961 | 26.925 | 1.00 | 38.16 |
| 2468 | N | ALA | B | 442 | 35.949 | 96.118 | 27.136 | 1.00 | 37.20 |
| 2469 | CA | ALA | B | 442 | 34.861 | 95.864 | 28.063 | 1.00 | 37.26 |
| 2470 | CB | ALA | B | 442 | 34.312 | 97.187 | 28.574 | 1.00 | 37.47 |
| 2471 | C | ALA | B | 442 | 33.718 | 95.032 | 27.483 | 1.00 | 39.85 |
| 2472 | O | ALA | B | 442 | 33.510 | 94.988 | 26.275 | 1.00 | 41.57 |
| 2473 | N | PRO | B | 443 | 32.960 | 94.354 | 28.357 | 1.00 | 39.83 |
| 2474 | CD | PRO | B | 443 | 33.303 | 94.122 | 29.766 | 1.00 | 40.96 |
| 2475 | CA | PRO | B | 443 | 31.825 | 93.520 | 27.969 | 1.00 | 38.29 |
| 2476 | CB | PRO | B | 443 | 31.678 | 92.542 | 29.144 | 1.00 | 36.98 |
| 2477 | CG | PRO | B | 443 | 32.925 | 92.693 | 29.932 | 1.00 | 36.14 |
| 2478 | C | PRO | B | 443 | 30.563 | 94.380 | 27.828 | 1.00 | 40.19 |
| 2479 | O | PRO | B | 443 | 30.406 | 95.396 | 28.512 | 1.00 | 40.51 |
| 2480 | N | ALA | B | 444 | 29.673 | 93.964 | 26.937 | 1.00 | 39.07 |
| 2481 | CA | ALA | B | 444 | 28.412 | 94.658 | 26.724 | 1.00 | 36.58 |
| 2482 | CB | ALA | B | 444 | 28.228 | 94.995 | 25.240 | 1.00 | 40.22 |
| 2483 | C | ALA | B | 444 | 27.373 | 93.638 | 27.181 | 1.00 | 34.33 |
| 2484 | O | ALA | B | 444 | 27.358 | 92.491 | 26.702 | 1.00 | 30.65 |
| 2485 | N | VAL | B | 445 | 26.519 | 94.057 | 28.109 | 1.00 | 28.37 |
| 2486 | CA | VAL | B | 445 | 25.520 | 93.172 | 28.664 | 1.00 | 26.39 |
| 2487 | CB | VAL | B | 445 | 25.652 | 93.152 | 30.198 | 1.00 | 26.69 |
| 2488 | CG1 | VAL | B | 445 | 24.620 | 92.205 | 30.790 | 1.00 | 23.97 |
| 2489 | CG2 | VAL | B | 445 | 27.076 | 92.747 | 30.586 | 1.00 | 22.56 |
| 2490 | C | VAL | B | 445 | 24.068 | 93.497 | 28.291 | 1.00 | 27.26 |
| 2491 | O | VAL | B | 445 | 23.646 | 94.644 | 28.367 | 1.00 | 24.12 |
| 2492 | N | TYR | B | 446 | 23.319 | 92.473 | 27.893 | 1.00 | 23.45 |
| 2493 | CA | TYR | B | 446 | 21.919 | 92.645 | 27.541 | 1.00 | 25.54 |
| 2494 | CB | TYR | B | 446 | 21.722 | 92.732 | 26.015 | 1.00 | 24.97 |
| 2495 | CG | TYR | B | 446 | 20.264 | 92.896 | 25.660 | 1.00 | 24.95 |
| 2496 | CD1 | TYR | B | 446 | 19.517 | 93.961 | 26.182 | 1.00 | 37.39 |
| 2497 | CE1 | TYR | B | 446 | 18.146 | 94.094 | 25.907 | 1.00 | 34.09 |
| 2498 | CD2 | TYR | B | 446 | 19.613 | 91.973 | 24.851 | 1.00 | 28.20 |
| 2499 | CE2 | TYR | B | 446 | 18.245 | 92.092 | 24.567 | 1.00 | 31.02 |
| 2500 | CZ | TYR | B | 446 | 17.519 | 93.160 | 25.102 | 1.00 | 34.30 |
| 2501 | OH | TYR | B | 446 | 16.174 | 93.294 | 24.846 | 1.00 | 35.42 |
| 2502 | C | TYR | B | 446 | 21.130 | 91.465 | 28.088 | 1.00 | 23.87 |
| 2503 | O | TYR | B | 446 | 21.321 | 90.325 | 27.663 | 1.00 | 28.38 |
| 2504 | N | ALA | B | 447 | 20.254 | 91.742 | 29.042 | 1.00 | 26.40 |
| 2505 | CA | ALA | B | 447 | 19.445 | 90.707 | 29.671 | 1.00 | 27.30 |
| 2506 | CB | ALA | B | 447 | 19.535 | 90.834 | 31.195 | 1.00 | 24.55 |
| 2507 | C | ALA | B | 447 | 18.002 | 90.840 | 29.202 | 1.00 | 30.87 |
| 2508 | O | ALA | B | 447 | 17.515 | 91.945 | 28.979 | 1.00 | 32.42 |
| 2509 | N | PHE | B | 448 | 17.310 | 89.720 | 29.045 | 1.00 | 31.33 |
| 2510 | CA | PHE | B | 448 | 15.939 | 89.799 | 28.582 | 1.00 | 34.25 |
| 2511 | CB | PHE | B | 448 | 15.938 | 89.984 | 27.060 | 1.00 | 40.48 |
| 2512 | CG | PHE | B | 448 | 16.497 | 88.805 | 26.314 | 1.00 | 39.26 |
| 2513 | CD1 | PHE | B | 448 | 15.663 | 87.763 | 25.923 | 1.00 | 39.27 |
| 2514 | CD2 | PHE | B | 448 | 17.862 | 88.704 | 26.059 | 1.00 | 36.97 |
| 2515 | CE1 | PHE | B | 448 | 16.180 | 86.633 | 25.286 | 1.00 | 43.41 |
| 2516 | CE2 | PHE | B | 448 | 18.389 | 87.578 | 25.422 | 1.00 | 36.78 |
| 2517 | CZ | PHE | B | 448 | 17.546 | 86.541 | 25.036 | 1.00 | 37.14 |
| 2518 | C | PHE | B | 448 | 15.113 | 88.579 | 28.952 | 1.00 | 34.75 |
| 2519 | O | PHE | B | 448 | 15.646 | 87.554 | 29.369 | 1.00 | 34.97 |
| 2520 | N | ALA | B | 449 | 13.801 | 88.708 | 28.798 | 1.00 | 37.02 |
| 2521 | CA | ALA | B | 449 | 12.877 | 87.623 | 29.079 | 1.00 | 38.65 |
| 2522 | CB | ALA | B | 449 | 11.731 | 88.122 | 29.898 | 1.00 | 32.34 |
| 2523 | C | ALA | B | 449 | 12.350 | 87.050 | 27.767 | 1.00 | 42.92 |
| 2524 | O | ALA | B | 449 | 12.165 | 87.774 | 26.794 | 1.00 | 42.07 |
| 2525 | N | THR | B | 450 | 12.111 | 85.743 | 27.759 | 1.00 | 47.71 |
| 2526 | CA | THR | B | 450 | 11.586 | 85.048 | 26.597 | 1.00 | 52.16 |
| 2527 | CB | THR | B | 450 | 12.040 | 83.577 | 26.599 | 1.00 | 53.90 |
| 2528 | OG1 | THR | B | 450 | 13.468 | 83.522 | 26.685 | 1.00 | 61.82 |
| 2529 | CG2 | THR | B | 450 | 11.594 | 82.877 | 25.327 | 1.00 | 62.58 |
| 2530 | C | THR | B | 450 | 10.053 | 85.097 | 26.607 | 1.00 | 54.69 |
| 2531 | O | THR | B | 450 | 9.421 | 84.983 | 27.659 | 1.00 | 51.28 |
| 2532 | N | PRO | B | 451 | 9.439 | 85.280 | 25.431 | 1.00 | 57.24 |
| 2533 | CD | PRO | B | 451 | 10.061 | 85.618 | 24.137 | 1.00 | 60.66 |
| 2534 | CA | PRO | B | 451 | 7.977 | 85.339 | 25.338 | 1.00 | 61.04 |
| 2535 | CB | PRO | B | 451 | 7.736 | 85.571 | 23.846 | 1.00 | 61.38 |
| 2536 | CG | PRO | B | 451 | 8.946 | 86.360 | 23.433 | 1.00 | 63.63 |
| 2537 | C | PRO | B | 451 | 7.301 | 84.063 | 25.847 | 1.00 | 63.11 |
| 2538 | O | PRO | B | 451 | 7.682 | 82.949 | 25.484 | 1.00 | 61.66 |
| 2539 | N | GLU | B | 452 | 6.303 | 84.230 | 26.706 | 1.00 | 66.04 |
| 2540 | CA | GLU | B | 452 | 5.575 | 83.083 | 27.237 | 1.00 | 69.40 |
| 2541 | CB | GLU | B | 452 | 5.800 | 82.954 | 28.753 | 1.00 | 67.34 |
| 2542 | CG | GLU | B | 452 | 5.911 | 81.505 | 29.277 | 1.00 | 60.86 |
| 2543 | CD | GLU | B | 452 | 7.225 | 80.815 | 28.876 | 1.00 | 59.08 |
| 2544 | OE1 | GLU | B | 452 | 8.255 | 81.510 | 28.775 | 1.00 | 62.62 |
| 2545 | OE2 | GLU | B | 452 | 7.249 | 79.578 | 28.676 | 1.00 | 45.82 |
| 2546 | C | GLU | B | 452 | 4.090 | 83.278 | 26.930 | 1.00 | 71.98 |
| 2547 | O | GLU | B | 452 | 3.360 | 83.895 | 27.705 | 1.00 | 70.35 |
| 2548 | N | ALA | B | 453 | 3.659 | 82.758 | 25.783 | 1.00 | 75.33 |
| 2549 | CA | ALA | B | 453 | 2.266 | 82.867 | 25.347 | 1.00 | 78.80 |
| 2550 | CB | ALA | B | 453 | 2.075 | 82.118 | 24.029 | 1.00 | 80.74 |
| 2551 | C | ALA | B | 453 | 1.282 | 82.340 | 26.394 | 1.00 | 80.53 |
| 2552 | O | ALA | B | 453 | 1.599 | 81.433 | 27.167 | 1.00 | 82.95 |
| 2553 | N | LYS | B | 459 | 8.685 | 75.798 | 30.647 | 1.00 | 51.87 |
| 2554 | CA | LYS | B | 459 | 9.250 | 76.647 | 31.693 | 1.00 | 52.84 |
| 2555 | CB | LYS | B | 459 | 10.479 | 75.987 | 32.317 | 1.00 | 55.20 |
| 2556 | CG | LYS | B | 459 | 10.171 | 74.721 | 33.089 | 1.00 | 63.98 |
| 2557 | CD | LYS | B | 459 | 11.271 | 74.401 | 34.088 | 1.00 | 72.67 |
| 2558 | CE | LYS | B | 459 | 11.366 | 75.484 | 35.157 | 1.00 | 77.63 |
| 2559 | NZ | LYS | B | 459 | 12.302 | 75.122 | 36.259 | 1.00 | 83.78 |
| 2560 | C | LYS | B | 459 | 9.638 | 78.033 | 31.195 | 1.00 | 50.86 |
| 2561 | O | LYS | B | 459 | 10.211 | 78.174 | 30.118 | 1.00 | 49.65 |
| 2562 | N | ARG | B | 460 | 9.332 | 79.045 | 32.001 | 1.00 | 48.30 |
| 2563 | CA | ARG | B | 460 | 9.640 | 80.433 | 31.674 | 1.00 | 47.61 |
| 2564 | CB | ARG | B | 460 | 8.865 | 81.350 | 32.620 | 1.00 | 51.65 |
| 2565 | CG | ARG | B | 460 | 7.371 | 81.029 | 32.591 | 1.00 | 59.85 |
| 2566 | CD | ARG | B | 460 | 6.570 | 81.784 | 33.635 | 1.00 | 65.41 |
| 2567 | NE | ARG | B | 460 | 5.158 | 81.400 | 33.603 | 1.00 | 70.36 |
| 2568 | CZ | ARG | B | 460 | 4.702 | 80.174 | 33.848 | 1.00 | 73.06 |
| 2569 | NH1 | ARG | B | 460 | 5.540 | 79.188 | 34.150 | 1.00 | 75.61 |
| 2570 | NH2 | ARG | B | 460 | 3.401 | 79.929 | 33.791 | 1.00 | 76.52 |
| 2571 | C | ARG | B | 460 | 11.154 | 80.664 | 31.759 | 1.00 | 44.39 |
| 2572 | O | ARG | B | 460 | 11.839 | 80.058 | 32.589 | 1.00 | 41.11 |
| 2573 | N | THR | B | 461 | 11.672 | 81.540 | 30.905 | 1.00 | 40.18 |
| 2574 | CA | THR | B | 461 | 13.111 | 81.768 | 30.850 | 1.00 | 40.02 |
| 2575 | CB | THR | B | 461 | 13.733 | 80.985 | 29.659 | 1.00 | 41.84 |
| 2576 | OG1 | THR | B | 461 | 13.383 | 79.601 | 29.750 | 1.00 | 52.98 |
| 2577 | CG2 | THR | B | 461 | 15.238 | 81.102 | 29.666 | 1.00 | 48.91 |
| 2578 | C | THR | B | 461 | 13.574 | 83.207 | 30.699 | 1.00 | 37.19 |
| 2579 | O | THR | B | 461 | 12.959 | 84.009 | 29.982 | 1.00 | 37.94 |
| 2580 | N | LEU | B | 462 | 14.681 | 83.512 | 31.375 | 1.00 | 33.00 |
| 2581 | CA | LEU | B | 462 | 15.335 | 84.809 | 31.289 | 1.00 | 29.46 |
| 2582 | CB | LEU | B | 462 | 15.550 | 85.434 | 32.667 | 1.00 | 30.35 |
| 2583 | CG | LEU | B | 462 | 14.282 | 85.678 | 33.498 | 1.00 | 33.09 |
| 2584 | CD1 | LEU | B | 462 | 14.639 | 86.412 | 34.785 | 1.00 | 32.32 |
| 2585 | CD2 | LEU | B | 462 | 13.293 | 86.499 | 32.699 | 1.00 | 25.52 |
| 2586 | C | LEU | B | 462 | 16.672 | 84.439 | 30.662 | 1.00 | 29.44 |
| 2587 | O | LEU | B | 462 | 17.184 | 83.339 | 30.894 | 1.00 | 24.62 |
| 2588 | N | ALA | B | 463 | 17.237 | 85.343 | 29.868 | 1.00 | 27.23 |
| 2589 | CA | ALA | B | 463 | 18.486 | 85.050 | 29.192 | 1.00 | 27.78 |
| 2590 | CB | ALA | B | 463 | 18.199 | 84.488 | 27.790 | 1.00 | 26.61 |
| 2591 | C | ALA | B | 463 | 19.332 | 86.301 | 29.104 | 1.00 | 28.37 |
| 2592 | O | ALA | B | 463 | 18.824 | 87.415 | 29.233 | 1.00 | 29.09 |
| 2593 | N | CYS | B | 464 | 20.623 | 86.115 | 28.870 | 1.00 | 22.40 |
| 2594 | CA | CYS | B | 464 | 21.534 | 87.246 | 28.821 | 1.00 | 26.72 |
| 2595 | C | CYS | B | 464 | 22.622 | 87.038 | 27.786 | 1.00 | 24.16 |
| 2596 | O | CYS | B | 464 | 23.199 | 85.952 | 27.691 | 1.00 | 23.01 |
| 2597 | CB | CYS | B | 464 | 22.172 | 87.410 | 30.200 | 1.00 | 24.58 |
| 2598 | SG | CYS | B | 464 | 23.327 | 88.794 | 30.423 | 1.00 | 40.31 |
| 2599 | N | LEU | B | 465 | 22.908 | 88.096 | 27.032 | 1.00 | 22.68 |
| 2600 | CA | LEU | B | 465 | 23.950 | 88.052 | 26.013 | 1.00 | 24.31 |
| 2601 | CB | LEU | B | 465 | 23.390 | 88.473 | 24.640 | 1.00 | 26.81 |
| 2602 | CG | LEU | B | 465 | 24.419 | 88.841 | 23.554 | 1.00 | 21.10 |
| 2603 | CD1 | LEU | B | 465 | 25.310 | 87.659 | 23.251 | 1.00 | 21.73 |
| 2604 | CD2 | LEU | B | 465 | 23.700 | 89.262 | 22.293 | 1.00 | 30.90 |
| 2605 | C | LEU | B | 465 | 25.043 | 89.013 | 26.453 | 1.00 | 23.79 |
| 2606 | O | LEU | B | 465 | 24.770 | 90.183 | 26.741 | 1.00 | 22.87 |
| 2607 | N | ILE | B | 466 | 26.276 | 88.521 | 26.510 | 1.00 | 23.16 |
| 2608 | CA | ILE | B | 466 | 27.402 | 89.349 | 26.926 | 1.00 | 23.50 |
| 2609 | CB | ILE | B | 466 | 28.018 | 88.838 | 28.251 | 1.00 | 23.66 |
| 2610 | CG2 | ILE | B | 466 | 29.140 | 89.800 | 28.705 | 1.00 | 29.47 |
| 2611 | CG1 | ILE | B | 466 | 26.913 | 88.739 | 29.320 | 1.00 | 19.89 |
| 2612 | CG1 | ILE | B | 466 | 27.398 | 88.229 | 30.650 | 1.00 | 36.91 |
| 2613 | C | ILE | B | 466 | 28.388 | 89.274 | 25.784 | 1.00 | 22.24 |
| 2614 | O | ILE | B | 466 | 28.811 | 88.189 | 25.385 | 1.00 | 24.74 |
| 2615 | N | GLN | B | 467 | 28.789 | 90.433 | 25.270 | 1.00 | 23.91 |
| 2616 | CA | GLN | B | 467 | 29.642 | 90.418 | 24.095 | 1.00 | 31.01 |
| 2617 | CB | GLN | B | 467 | 28.747 | 90.376 | 22.846 | 1.00 | 30.11 |
| 2618 | CG | GLN | B | 467 | 27.869 | 91.607 | 22.654 | 1.00 | 26.37 |
| 2619 | CD | GLN | B | 467 | 26.920 | 91.486 | 21.464 | 1.00 | 29.83 |
| 2620 | OE1 | GLN | B | 467 | 27.136 | 90.710 | 20.521 | 1.00 | 28.59 |
| 2621 | NE2 | GLN | B | 467 | 25.869 | 92.282 | 21.495 | 1.00 | 31.49 |
| 2622 | C | GLN | B | 467 | 30.641 | 91.546 | 23.952 | 1.00 | 33.72 |
| 2623 | O | GLN | B | 467 | 30.691 | 92.469 | 24.768 | 1.00 | 31.53 |
| 2624 | N | ASN | B | 468 | 31.442 | 91.421 | 22.895 | 1.00 | 38.27 |
| 2625 | CA | ASN | B | 468 | 32.465 | 92.385 | 22.526 | 1.00 | 41.08 |
| 2626 | CB | ASN | B | 468 | 31.839 | 93.744 | 22.245 | 1.00 | 43.70 |
| 2627 | CG | ASN | B | 468 | 30.665 | 93.651 | 21.303 | 1.00 | 50.48 |
| 2628 | OD1 | ASN | B | 468 | 30.722 | 92.959 | 20.281 | 1.00 | 59.45 |
| 2629 | ND2 | ASN | B | 468 | 29.594 | 94.357 | 21.628 | 1.00 | 56.28 |
| 2630 | C | ASN | B | 468 | 33.560 | 92.548 | 23.554 | 1.00 | 41.04 |
| 2631 | O | ASN | B | 468 | 34.155 | 93.614 | 23.660 | 1.00 | 38.53 |
| 2632 | N | PHE | B | 469 | 33.843 | 91.499 | 24.309 | 1.00 | 38.66 |
| 2633 | CA | PHE | B | 469 | 34.894 | 91.604 | 25.303 | 1.00 | 38.07 |
| 2634 | CB | PHE | B | 469 | 34.360 | 91.181 | 26.673 | 1.00 | 29.35 |
| 2635 | CG | PHE | B | 469 | 34.064 | 89.712 | 26.791 | 1.00 | 28.48 |
| 2636 | CG1 | PHE | B | 469 | 35.059 | 88.815 | 27.113 | 1.00 | 23.56 |
| 2637 | CD2 | PHE | B | 469 | 32.777 | 89.231 | 26.579 | 1.00 | 24.16 |
| 2638 | CE1 | PHE | B | 469 | 34.784 | 87.463 | 27.228 | 1.00 | 27.41 |
| 2639 | CE2 | PHE | B | 469 | 32.493 | 87.867 | 26.691 | 1.00 | 32.69 |
| 2640 | CZ | PHE | B | 469 | 33.506 | 86.984 | 27.017 | 1.00 | 24.40 |
| 2641 | C | PHE | B | 469 | 36.125 | 90.785 | 24.939 | 1.00 | 38.11 |
| 2642 | O | PHE | B | 469 | 36.026 | 89.752 | 24.287 | 1.00 | 39.11 |
| 2643 | N | MET | B | 470 | 37.287 | 91.267 | 25.359 | 1.00 | 40.05 |
| 2644 | CA | MET | B | 470 | 38.569 | 90.599 | 25.130 | 1.00 | 41.39 |
| 2645 | CB | MET | B | 470 | 39.183 | 90.982 | 23.779 | 1.00 | 46.13 |
| 2646 | CG | MET | B | 470 | 39.557 | 92.441 | 23.570 | 1.00 | 60.40 |
| 2647 | SD | MET | B | 470 | 39.966 | 92.737 | 21.820 | 1.00 | 65.64 |
| 2648 | CE | MET | B | 470 | 41.659 | 92.059 | 21.727 | 1.00 | 71.36 |
| 2649 | C | MET | B | 470 | 39.417 | 91.095 | 26.280 | 1.00 | 38.04 |
| 2650 | O | MET | B | 470 | 39.376 | 92.273 | 26.618 | 1.00 | 36.87 |
| 2651 | N | PRO | B | 471 | 40.184 | 90.201 | 26.925 | 1.00 | 37.84 |
| 2652 | CD | PRO | B | 471 | 40.913 | 90.654 | 28.117 | 1.00 | 38.40 |
| 2653 | CA | PRO | B | 471 | 40.360 | 88.747 | 26.742 | 1.00 | 37.04 |
| 2654 | CB | PRO | B | 471 | 41.485 | 88.400 | 27.732 | 1.00 | 38.82 |
| 2655 | CG | PRO | B | 471 | 42.076 | 89.748 | 28.096 | 1.00 | 40.26 |
| 2656 | C | PRO | B | 471 | 39.092 | 87.915 | 26.996 | 1.00 | 35.06 |
| 2657 | O | PRO | B | 471 | 38.076 | 88.443 | 27.432 | 1.00 | 33.27 |
| 2658 | N | GLU | B | 472 | 39.180 | 86.611 | 26.760 | 1.00 | 35.63 |
| 2659 | CA | GLU | B | 472 | 38.033 | 85.705 | 26.898 | 1.00 | 38.83 |
| 2660 | CB | GLU | B | 472 | 38.292 | 84.431 | 26.109 | 1.00 | 43.92 |
| 2661 | CG | GLU | B | 472 | 39.299 | 83.516 | 26.792 | 1.00 | 54.98 |
| 2662 | CD | GLU | B | 472 | 39.657 | 82.288 | 25.963 | 1.00 | 65.38 |
| 2663 | OE1 | GLU | B | 472 | 39.172 | 82.169 | 24.821 | 1.00 | 73.96 |
| 2664 | OE2 | GLU | B | 472 | 40.433 | 81.442 | 26.450 | 1.00 | 71.17 |
| 2665 | C | GLU | B | 472 | 37.568 | 85.292 | 28.294 | 1.00 | 36.65 |
| 2666 | O | GLU | B | 472 | 36.453 | 84.801 | 28.451 | 1.00 | 32.41 |
| 2667 | N | ASP | B | 473 | 38.402 | 85.482 | 29.311 | 1.00 | 35.48 |
| 2668 | CA | ASP | B | 473 | 38.004 | 85.092 | 30.668 | 1.00 | 34.00 |
| 2669 | CB | ASP | B | 473 | 39.225 | 85.066 | 31.594 | 1.00 | 39.24 |
| 2670 | CG | ASP | B | 473 | 40.239 | 83.993 | 31.193 | 1.00 | 48.86 |
| 2671 | OD1 | ASP | B | 473 | 39.800 | 82.876 | 30.817 | 1.00 | 42.86 |
| 2672 | OD2 | ASP | B | 473 | 41.461 | 84.270 | 31.265 | 1.00 | 46.87 |
| 2673 | C | ASP | B | 473 | 36.917 | 85.993 | 31.256 | 1.00 | 34.33 |
| 2674 | O | ASP | B | 473 | 37.057 | 87.220 | 31.315 | 1.00 | 34.18 |
| 2675 | N | TLE | B | 474 | 35.824 | 85.384 | 31.691 | 1.00 | 27.93 |
| 2676 | CA | ILE | B | 474 | 34.738 | 86.165 | 32.254 | 1.00 | 28.40 |
| 2677 | CB | ILE | B | 474 | 33.848 | 86.726 | 31.129 | 1.00 | 25.16 |
| 2678 | CG2 | ILE | B | 474 | 33.036 | 85.602 | 30.489 | 1.00 | 26.53 |
| 2679 | CG1 | ILE | B | 474 | 32.935 | 87.828 | 31.664 | 1.00 | 21.56 |
| 2680 | CD1 | ILE | B | 474 | 32.219 | 88.591 | 30.507 | 1.00 | 23.93 |
| 2681 | C | ILE | B | 474 | 33.890 | 85.326 | 33.190 | 1.00 | 26.83 |
| 2682 | O | ILE | B | 474 | 33.773 | 84.127 | 33.014 | 1.00 | 25.94 |
| 2683 | N | SER | B | 475 | 33.325 | 85.975 | 34.202 | 1.00 | 24.54 |
| 2684 | CA | SER | B | 475 | 32.446 | 85.336 | 35.167 | 1.00 | 23.71 |
| 2685 | CB | SER | B | 475 | 32.932 | 85.586 | 36.590 | 1.00 | 22.00 |
| 2686 | OG | SER | B | 475 | 34.221 | 85.045 | 36.759 | 1.00 | 30.00 |
| 2687 | C | SER | B | 475 | 31.071 | 85.967 | 34.983 | 1.00 | 24.55 |
| 2688 | O | SER | B | 475 | 30.931 | 87.190 | 34.965 | 1.00 | 23.24 |
| 2689 | N | VAL | B | 476 | 30.058 | 85.124 | 34.847 | 1.00 | 24.48 |
| 2690 | CA | VAL | B | 476 | 28.692 | 85.562 | 34.654 | 1.00 | 26.25 |
| 2691 | CB | VAL | B | 476 | 28.097 | 84.928 | 33.379 | 1.00 | 24.14 |
| 2692 | CG1 | VAL | B | 476 | 26.635 | 85.340 | 33.227 | 1.00 | 23.36 |
| 2693 | CG2 | VAL | B | 476 | 28.907 | 85.357 | 32.163 | 1.00 | 28.16 |
| 2694 | C | VAL | B | 476 | 27.877 | 85.111 | 35.847 | 1.00 | 28.80 |
| 2695 | O | VAL | B | 476 | 28.045 | 83.993 | 36.339 | 1.00 | 32.13 |
| 2696 | N | GLN | B | 477 | 26.993 | 85.971 | 36.326 | 1.00 | 28.79 |
| 2697 | CA | GLN | B | 477 | 26.176 | 85.589 | 37.458 | 1.00 | 30.02 |
| 2698 | CB | GLN | B | 477 | 26.913 | 85.854 | 38.774 | 1.00 | 31.30 |
| 2699 | CG | GLN | B | 477 | 27.360 | 87.270 | 38.993 | 1.00 | 46.28 |
| 2700 | CD | GLN | B | 477 | 28.499 | 87.368 | 40.007 | 1.00 | 49.95 |
| 2701 | OE1 | GLN | B | 477 | 28.842 | 88.455 | 40.465 | 1.00 | 51.94 |
| 2702 | NE2 | GLN | B | 477 | 29.097 | 86.227 | 40.349 | 1.00 | 51.51 |
| 2703 | C | GLN | B | 477 | 24.856 | 86.313 | 37.431 | 1.00 | 30.41 |
| 2704 | O | GLN | B | 477 | 24.757 | 87.415 | 36.902 | 1.00 | 30.03 |
| 2705 | N | TRP | B | 478 | 23.841 | 85.659 | 37.976 | 1.00 | 29.86 |
| 2706 | CA | TRP | B | 478 | 22.505 | 86.210 | 38.042 | 1.00 | 29.12 |
| 2707 | CB | TRP | B | 478 | 21.499 | 85.165 | 37.579 | 1.00 | 31.11 |
| 2708 | CG | TRP | B | 478 | 21.527 | 84.936 | 36.112 | 1.00 | 36.98 |
| 2709 | CD2 | TRP | B | 478 | 20.726 | 85.605 | 35.132 | 1.00 | 34.77 |
| 2710 | CE2 | TRP | B | 478 | 21.092 | 85.084 | 33.870 | 1.00 | 37.75 |
| 2711 | CE3 | TRP | B | 478 | 19.742 | 86.602 | 35.194 | 1.00 | 31.61 |
| 2712 | CD1 | TRP | B | 478 | 22.320 | 84.060 | 35.432 | 1.00 | 35.86 |
| 2713 | NE1 | TRP | B | 478 | 22.065 | 84.142 | 34.086 | 1.00 | 35.58 |
| 2714 | CZ2 | TRP | B | 478 | 20.494 | 85.521 | 32.668 | 1.00 | 26.96 |
| 2715 | CZ3 | TRP | B | 478 | 19.148 | 87.039 | 34.005 | 1.00 | 35.86 |
| 2716 | CH2 | TRP | B | 478 | 19.531 | 86.497 | 32.757 | 1.00 | 31.66 |
| 2717 | C | TRP | B | 478 | 22.175 | 86.648 | 39.468 | 1.00 | 29.01 |
| 2718 | O | TRP | B | 478 | 22.580 | 86.006 | 40.439 | 1.00 | 24.19 |
| 2719 | N | LEU | B | 479 | 21.436 | 87.742 | 39.582 | 1.00 | 31.15 |
| 2720 | CA | LEU | B | 479 | 21.042 | 88.275 | 40.880 | 1.00 | 36.67 |
| 2721 | CB | LEU | B | 479 | 21.837 | 89.537 | 41.198 | 1.00 | 41.07 |
| 2722 | CG | LEU | B | 479 | 23.328 | 89.584 | 40.867 | 1.00 | 48.71 |
| 2723 | CD1 | LEU | B | 479 | 23.889 | 90.861 | 41.440 | 1.00 | 57.57 |
| 2724 | CD2 | LEU | B | 479 | 24.054 | 88.396 | 41.455 | 1.00 | 56.00 |
| 2725 | C | LEU | B | 479 | 19.556 | 88.624 | 40.920 | 1.00 | 38.99 |
| 2726 | O | LEU | B | 479 | 18.965 | 88.998 | 39.909 | 1.00 | 37.96 |
| 2727 | N | HIS | B | 480 | 18.963 | 88.500 | 42.096 | 1.00 | 42.00 |
| 2728 | CA | HIS | B | 480 | 17.561 | 88.834 | 42.284 | 1.00 | 48.67 |
| 2729 | CB | HIS | B | 480 | 16.784 | 87.648 | 42.871 | 1.00 | 50.22 |
| 2730 | CG | HIS | B | 480 | 15.309 | 87.894 | 43.014 | 1.00 | 53.99 |
| 2731 | CD2 | HIS | B | 480 | 14.256 | 87.041 | 42.993 | 1.00 | 56.80 |
| 2732 | ND1 | HIS | B | 480 | 14.779 | 89.147 | 43.239 | 1.00 | 54.56 |
| 2733 | CE1 | HIS | B | 480 | 13.465 | 89.057 | 43.348 | 1.00 | 60.76 |
| 2734 | NE2 | HIS | B | 480 | 13.122 | 87.789 | 43.203 | 1.00 | 59.92 |
| 2735 | C | HIS | B | 480 | 17.625 | 89.972 | 43.283 | 1.00 | 52.66 |
| 2736 | O | HIS | B | 480 | 17.426 | 89.777 | 44.482 | 1.00 | 51.85 |
| 2737 | N | ASN | B | 481 | 17.945 | 91.161 | 42.780 | 1.00 | 58.88 |
| 2738 | CA | ASN | B | 481 | 18.062 | 92.339 | 43.628 | 1.00 | 63.74 |
| 2739 | CB | ASN | B | 481 | 16.763 | 92.588 | 44.407 | 1.00 | 69.70 |
| 2740 | CG | ASN | B | 481 | 15.674 | 93.211 | 43.561 | 1.00 | 76.04 |
| 2741 | OD1 | ASN | B | 481 | 14.513 | 93.246 | 43.962 | 1.00 | 80.67 |
| 2742 | ND2 | ASN | B | 481 | 16.042 | 93.719 | 42.392 | 1.00 | 82.88 |
| 2743 | C | ASN | B | 481 | 19.182 | 92.134 | 44.633 | 1.00 | 63.93 |
| 2744 | O | ASN | B | 481 | 19.008 | 91.402 | 45.604 | 1.00 | 67.09 |
| 2745 | N | GLU | B | 482 | 20.322 | 92.781 | 44.399 | 1.00 | 62.43 |
| 2746 | CA | GLU | B | 482 | 21.477 | 92.715 | 45.299 | 1.00 | 60.15 |
| 2747 | CB | GLU | B | 482 | 21.225 | 93.623 | 46.519 | 1.00 | 65.85 |
| 2748 | CG | GLU | B | 482 | 19.754 | 94.021 | 46.707 | 1.00 | 78.14 |
| 2749 | CD | GLU | B | 482 | 19.282 | 93.946 | 48.144 | 1.00 | 84.88 |
| 2750 | OE1 | GLU | B | 482 | 19.662 | 92.984 | 48.846 | 1.00 | 90.19 |
| 2751 | OE2 | GLU | B | 482 | 18.518 | 94.841 | 48.566 | 1.00 | 87.19 |
| 2752 | C | GLU | B | 482 | 21.949 | 91.331 | 45.786 | 1.00 | 54.93 |
| 2753 | O | GLU | B | 482 | 23.084 | 91.194 | 46.247 | 1.00 | 57.04 |
| 2754 | N | VAL | B | 483 | 21.111 | 90.305 | 45.686 | 1.00 | 49.03 |
| 2755 | CA | VAL | B | 483 | 21.511 | 88.987 | 46.163 | 1.00 | 45.46 |
| 2756 | CB | VAL | B | 483 | 20.450 | 88.391 | 47.126 | 1.00 | 43.71 |
| 2757 | CG1 | VAL | B | 483 | 19.099 | 88.280 | 46.427 | 1.00 | 50.19 |
| 2758 | CG2 | VAL | B | 483 | 20.913 | 87.033 | 47.633 | 1.00 | 43.76 |
| 2759 | C | VAL | B | 483 | 21.758 | 88.016 | 45.022 | 1.00 | 43.31 |
| 2760 | O | VAL | B | 483 | 20.924 | 87.859 | 44.137 | 1.00 | 41.21 |
| 2761 | N | CLN | B | 484 | 22.913 | 87.362 | 45.061 | 1.00 | 42.10 |
| 2762 | CA | GLN | B | 484 | 23.303 | 86.411 | 44.036 | 1.00 | 41.31 |
| 2763 | CB | CLN | B | 484 | 24.822 | 86.251 | 44.047 | 1.00 | 46.93 |
| 2764 | CG | GLN | B | 484 | 25.338 | 85.180 | 43.107 | 1.00 | 54.66 |
| 2765 | CD | GLN | B | 484 | 26.851 | 85.172 | 42.999 | 1.00 | 61.50 |
| 2766 | OE1 | GLN | B | 484 | 27.442 | 84.198 | 42.532 | 1.00 | 65.59 |
| 2767 | NE2 | GLN | B | 484 | 27.486 | 86.267 | 43.416 | 1.00 | 62.36 |
| 2768 | C | CLN | B | 484 | 22.642 | 85.036 | 44.141 | 1.00 | 40.69 |
| 2769 | O | GLN | B | 484 | 22.503 | 84.476 | 45.232 | 1.00 | 39.56 |
| 2770 | N | LEU | B | 485 | 22.242 | 84.502 | 42.989 | 1.00 | 34.74 |
| 2771 | CA | LEU | B | 485 | 21.602 | 83.192 | 42.909 | 1.00 | 36.73 |
| 2772 | CB | LEU | B | 485 | 20.721 | 83.089 | 41.655 | 1.00 | 36.78 |
| 2773 | CG | LEU | B | 485 | 19.589 | 84.099 | 41.457 | 1.00 | 38.16 |
| 2774 | CD1 | LEU | B | 485 | 18.876 | 83.831 | 40.134 | 1.00 | 39.22 |
| 2775 | CD2 | LEU | B | 485 | 18.631 | 83.993 | 42.621 | 1.00 | 41.68 |
| 2776 | C | LEU | B | 485 | 22.677 | 82.115 | 42.834 | 1.00 | 36.82 |
| 2777 | O | LEU | B | 485 | 23.805 | 82.388 | 42.440 | 1.00 | 34.53 |
| 2778 | N | PRO | B | 486 | 22.342 | 80.875 | 43.217 | 1.00 | 40.02 |
| 2779 | CD | PRO | B | 486 | 21.037 | 80.349 | 43.652 | 1.00 | 39.74 |
| 2780 | CA | PRO | B | 486 | 23.351 | 79.809 | 43.151 | 1.00 | 45.08 |
| 2781 | CB | PRO | B | 486 | 22.606 | 78.582 | 43.678 | 1.00 | 44.97 |
| 2782 | CG | PRO | B | 486 | 21.438 | 79.143 | 44.439 | 1.00 | 41.94 |
| 2783 | C | PRO | B | 486 | 23.738 | 79.629 | 41.687 | 1.00 | 49.91 |
| 2784 | O | PRO | B | 486 | 22.889 | 79.770 | 40.809 | 1.00 | 49.23 |
| 2785 | N | ASP | B | 487 | 25.001 | 79.309 | 41.423 | 1.00 | 54.02 |
| 2786 | CA | ASP | B | 487 | 25.469 | 79.114 | 40.054 | 1.00 | 58.50 |
| 2787 | CB | ASP | B | 487 | 26.953 | 78.749 | 40.061 | 1.00 | 68.77 |
| 2788 | CG | ASP | B | 487 | 27.607 | 78.938 | 38.708 | 1.00 | 79.77 |
| 2789 | OD1 | ASP | B | 487 | 27.055 | 78.436 | 37.703 | 1.00 | 87.08 |
| 2790 | OD2 | ASP | B | 487 | 28.679 | 79.584 | 38.652 | 1.00 | 87.04 |
| 2791 | C | ASP | B | 487 | 24.672 | 78.006 | 39.354 | 1.00 | 57.99 |
| 2792 | O | ASP | B | 487 | 24.248 | 78.155 | 38.202 | 1.00 | 58.25 |
| 2793 | N | ALA | B | 488 | 24.465 | 76.905 | 40.071 | 1.00 | 55.74 |
| 2794 | CA | ALA | B | 488 | 23.739 | 75.748 | 39.554 | 1.00 | 55.27 |
| 2795 | CB | ALA | B | 488 | 23.478 | 74.767 | 40.682 | 1.00 | 55.02 |
| 2796 | C | ALA | B | 488 | 22.423 | 76.089 | 38.861 | 1.00 | 54.82 |
| 2797 | O | ALA | B | 488 | 21.874 | 75.274 | 38.117 | 1.00 | 55.09 |
| 2798 | N | ARG | B | 489 | 21.918 | 77.293 | 39.096 | 1.00 | 52.93 |
| 2799 | CA | ARG | B | 489 | 20.656 | 77.701 | 38.501 | 1.00 | 48.90 |
| 2800 | CB | ARG | B | 489 | 20.029 | 78.801 | 39.351 | 1.00 | 47.45 |
| 2801 | CG | ARG | B | 489 | 19.257 | 78.264 | 40.537 | 1.00 | 49.40 |
| 2802 | CD | ARG | B | 489 | 17.772 | 78.337 | 40.256 | 1.00 | 52.76 |
| 2803 | NE | ARG | B | 489 | 17.174 | 79.497 | 40.910 | 1.00 | 60.57 |
| 2804 | CZ | ARG | B | 489 | 16.041 | 80.087 | 40.540 | 1.00 | 60.40 |
| 2805 | NH1 | ARG | B | 489 | 15.353 | 79.641 | 39.496 | 1.00 | 61.65 |
| 2806 | NH2 | ARG | B | 489 | 15.587 | 81.120 | 41.234 | 1.00 | 62.45 |
| 2807 | C | ARG | B | 489 | 20.706 | 78.144 | 37.042 | 1.00 | 45.96 |
| 2808 | O | ARG | B | 489 | 19.688 | 78.099 | 36.354 | 1.00 | 45.86 |
| 2809 | N | HIS | B | 490 | 21.867 | 78.568 | 36.559 | 1.00 | 41.69 |
| 2810 | CA | HIS | B | 490 | 21.945 | 79.020 | 35.172 | 1.00 | 38.41 |
| 2811 | CB | HIS | B | 490 | 22.314 | 80.513 | 35.103 | 1.00 | 39.34 |
| 2812 | CG | HIS | B | 490 | 23.703 | 80.825 | 35.570 | 1.00 | 38.03 |
| 2813 | CD2 | HIS | B | 490 | 24.885 | 80.816 | 34.911 | 1.00 | 41.49 |
| 2814 | ND1 | HIS | B | 490 | 23.994 | 81.157 | 36.876 | 1.00 | 44.03 |
| 2815 | CE1 | HIS | B | 490 | 25.298 | 81.337 | 37.001 | 1.00 | 42.68 |
| 2816 | NE2 | HIS | B | 490 | 25.860 | 81.135 | 35.823 | 1.00 | 46.75 |
| 2817 | C | HIS | B | 490 | 22.921 | 78.218 | 34.322 | 1.00 | 37.56 |
| 2818 | O | HIS | B | 490 | 23.748 | 77.483 | 34.840 | 1.00 | 38.74 |
| 2819 | N | SER | B | 491 | 22.804 | 78.359 | 33.006 | 1.00 | 36.46 |
| 2820 | CA | SER | B | 491 | 23.694 | 77.674 | 32.079 | 1.00 | 35.63 |
| 2821 | CB | SER | B | 491 | 22.902 | 76.660 | 31.255 | 1.00 | 34.76 |
| 2822 | OG | SER | B | 491 | 23.751 | 75.950 | 30.374 | 1.00 | 51.24 |
| 2823 | C | SER | B | 491 | 24.355 | 78.712 | 31.165 | 1.00 | 32.46 |
| 2824 | O | SER | B | 491 | 23.672 | 79.478 | 30.482 | 1.00 | 34.26 |
| 2825 | N | THR | B | 492 | 25.681 | 78.732 | 31.154 | 1.00 | 30.35 |
| 2826 | CA | THR | B | 492 | 26.453 | 79.678 | 30.350 | 1.00 | 32.31 |
| 2827 | CB | THR | B | 492 | 27.421 | 80.496 | 31.263 | 1.00 | 33.20 |
| 2828 | OG1 | THR | B | 492 | 26.667 | 81.141 | 32.294 | 1.00 | 38.93 |
| 2829 | CG2 | THR | B | 492 | 28.185 | 81.554 | 30.478 | 1.00 | 30.28 |
| 2830 | C | THR | B | 492 | 27.272 | 78.961 | 29.265 | 1.00 | 32.83 |
| 2831 | O | THR | B | 492 | 27.930 | 77.962 | 29.535 | 1.00 | 34.30 |
| 2832 | N | THR | B | 493 | 27.240 | 79.482 | 28.042 | 1.00 | 32.82 |
| 2833 | CA | THR | B | 493 | 27.990 | 78.881 | 26.939 | 1.00 | 32.09 |
| 2834 | CB | THR | B | 493 | 27.449 | 79.346 | 25.570 | 1.00 | 27.96 |
| 2835 | OG1 | THR | B | 493 | 27.541 | 80.775 | 25.470 | 1.00 | 29.42 |
| 2836 | CG2 | THR | B | 493 | 26.000 | 78.920 | 25.406 | 1.00 | 21.13 |
| 2837 | C | THR | B | 493 | 29.469 | 79.264 | 27.033 | 1.00 | 34.06 |
| 2838 | O | THR | B | 493 | 29.830 | 80.164 | 27.783 | 1.00 | 31.49 |
| 2839 | N | GLN | B | 494 | 30.318 | 78.570 | 26.284 | 1.00 | 35.84 |
| 2840 | CA | GLN | B | 494 | 31.752 | 78.860 | 26.276 | 1.00 | 40.51 |
| 2841 | CB | GLN | B | 494 | 32.544 | 77.671 | 25.718 | 1.00 | 46.41 |
| 2842 | CG | GLN | B | 494 | 32.522 | 76.431 | 26.596 | 1.00 | 63.89 |
| 2843 | CD | GLN | B | 494 | 33.008 | 76.710 | 28.007 | 1.00 | 72.04 |
| 2844 | OE1 | GLN | B | 494 | 34.053 | 77.328 | 28.206 | 1.00 | 77.06 |
| 2845 | NE2 | GLN | B | 494 | 32.250 | 76.245 | 28.996 | 1.00 | 81.58 |
| 2846 | C | GLN | B | 494 | 32.004 | 80.088 | 25.409 | 1.00 | 39.51 |
| 2847 | O | GLN | B | 494 | 31.330 | 80.296 | 24.402 | 1.00 | 38.74 |
| 2848 | N | PRO | B | 495 | 32.977 | 80.924 | 25.795 | 1.00 | 38.63 |
| 2849 | CD | PRO | B | 495 | 33.751 | 80.913 | 27.049 | 1.00 | 37.82 |
| 2850 | CA | PRO | B | 495 | 33.270 | 82.120 | 25.000 | 1.00 | 39.10 |
| 2851 | CB | PRO | B | 495 | 34.476 | 82.711 | 25.709 | 1.00 | 37.94 |
| 2852 | CG | PRO | B | 495 | 34.212 | 82.345 | 27.146 | 1.00 | 43.34 |
| 2853 | C | PRO | B | 495 | 33.569 | 81.746 | 23.551 | 1.00 | 41.43 |
| 2854 | O | PRO | B | 495 | 34.254 | 80.762 | 23.292 | 1.00 | 42.40 |
| 2855 | N | ARG | B | 496 | 33.033 | 82.514 | 22.613 | 1.00 | 41.33 |
| 2856 | CA | ARG | B | 496 | 33.257 | 82.257 | 21.194 | 1.00 | 46.41 |
| 2857 | CB | ARG | B | 496 | 31.965 | 81.792 | 20.526 | 1.00 | 44.11 |
| 2858 | CG | ARG | B | 496 | 31.657 | 80.337 | 20.768 | 1.00 | 51.48 |
| 2859 | CD | ARG | B | 496 | 30.269 | 79.967 | 20.287 | 1.00 | 56.73 |
| 2860 | NE | ARG | B | 496 | 30.207 | 78.562 | 19.903 | 1.00 | 63.41 |
| 2861 | CZ | ARG | B | 496 | 30.616 | 78.093 | 18.727 | 1.00 | 59.61 |
| 2862 | NH1 | ARG | B | 496 | 31.111 | 78.917 | 17.814 | 1.00 | 60.71 |
| 2863 | NH2 | ARG | B | 496 | 30.534 | 76.796 | 18.466 | 1.00 | 67.27 |
| 2864 | C | ARG | B | 496 | 33.765 | 83.510 | 20.503 | 1.00 | 49.11 |
| 2865 | O | ARG | B | 496 | 33.297 | 84.611 | 20.782 | 1.00 | 48.52 |
| 2866 | N | LYS | B | 497 | 34.730 | 83.345 | 19.608 | 1.00 | 53.35 |
| 2867 | CA | LYS | B | 497 | 35.278 | 84.484 | 18.892 | 1.00 | 60.05 |
| 2868 | CB | LYS | B | 497 | 36.499 | 84.073 | 18.066 | 1.00 | 65.19 |
| 2869 | CG | LYS | B | 497 | 37.713 | 83.690 | 18.888 | 1.00 | 75.27 |
| 2870 | CD | LYS | B | 497 | 38.895 | 83.345 | 17.997 | 1.00 | 80.74 |
| 2871 | CE | LYS | B | 497 | 40.130 | 83.066 | 18.831 | 1.00 | 86.89 |
| 2872 | NZ | LYS | B | 497 | 41.322 | 82.746 | 17.997 | 1.00 | 91.84 |
| 2873 | C | LYS | B | 497 | 34.224 | 85.054 | 17.969 | 1.00 | 62.05 |
| 2874 | O | LYS | B | 497 | 33.209 | 84.416 | 17.697 | 1.00 | 61.50 |
| 2875 | N | THR | B | 498 | 34.465 | 86.265 | 17.496 | 1.00 | 66.47 |
| 2876 | CA | THR | B | 498 | 33.540 | 86.910 | 16.585 | 1.00 | 71.47 |
| 2877 | CB | THR | B | 498 | 32.946 | 88.183 | 17.207 | 1.00 | 73.54 |
| 2878 | OG1 | THR | B | 498 | 33.619 | 88.482 | 18.434 | 1.00 | 77.08 |
| 2879 | CG2 | THR | B | 498 | 31.480 | 87.981 | 17.491 | 1.00 | 75.50 |
| 2880 | C | THR | B | 498 | 34.281 | 87.261 | 15.306 | 1.00 | 72.65 |
| 2881 | O | THR | B | 498 | 35.377 | 86.750 | 15.063 | 1.00 | 72.18 |
| 2882 | N | ALA | B | 499 | 33.682 | 88.123 | 14.490 | 1.00 | 75.02 |
| 2883 | CA | ALA | B | 499 | 34.297 | 88.538 | 13.233 | 1.00 | 77.01 |
| 2884 | CB | ALA | B | 499 | 33.230 | 89.092 | 12.286 | 1.00 | 76.94 |
| 2885 | C | ALA | B | 499 | 35.376 | 89.591 | 13.493 | 1.00 | 77.96 |
| 2886 | O | ALA | B | 499 | 35.501 | 90.566 | 12.750 | 1.00 | 79.10 |
| 2887 | N | GLY | B | 500 | 36.157 | 89.386 | 14.548 | 1.00 | 78.37 |
| 2888 | CA | GLY | B | 500 | 37.210 | 90.327 | 14.883 | 1.00 | 78.64 |
| 2889 | C | GLY | B | 500 | 36.855 | 91.208 | 16.068 | 1.00 | 77.71 |
| 2890 | O | GLY | B | 500 | 37.722 | 91.859 | 16.653 | 1.00 | 78.53 |
| 2891 | N | SER | B | 501 | 35.579 | 91.210 | 16.439 | 1.00 | 76.72 |
| 2892 | CA | SER | B | 501 | 35.099 | 92.033 | 17.545 | 1.00 | 74.10 |
| 2893 | CB | SER | B | 501 | 33.707 | 92.583 | 17.201 | 1.00 | 79.09 |
| 2894 | OG | SER | B | 501 | 32.813 | 91.547 | 16.824 | 1.00 | 82.51 |
| 2895 | C | SER | B | 501 | 35.055 | 91.365 | 18.924 | 1.00 | 70.47 |
| 2896 | O | SER | B | 501 | 34.025 | 91.412 | 19.601 | 1.00 | 71.67 |
| 2897 | N | GLY | B | 502 | 36.165 | 90.751 | 19.339 | 1.00 | 64.36 |
| 2898 | CA | GLY | B | 502 | 36.231 | 90.116 | 20.653 | 1.00 | 55.25 |
| 2899 | C | GLY | B | 502 | 35.489 | 88.801 | 20.889 | 1.00 | 48.43 |
| 2900 | O | GLY | B | 502 | 35.445 | 87.931 | 20.019 | 1.00 | 47.45 |
| 2901 | N | PHE | B | 503 | 34.922 | 88.648 | 22.085 | 1.00 | 41.96 |
| 2902 | CA | PHE | B | 503 | 34.189 | 87.425 | 22.435 | 1.00 | 34.38 |
| 2903 | CB | PHE | B | 503 | 34.925 | 86.625 | 23.518 | 1.00 | 34.97 |
| 2904 | CG | PHE | B | 503 | 36.296 | 86.168 | 23.121 | 1.00 | 28.11 |
| 2905 | CD1 | PHE | B | 503 | 37.368 | 87.052 | 23.126 | 1.00 | 36.44 |
| 2906 | CD2 | PHE | B | 503 | 36.518 | 84.851 | 22.752 | 1.00 | 30.75 |
| 2907 | CE1 | PHE | B | 503 | 38.640 | 86.629 | 22.762 | 1.00 | 37.43 |
| 2908 | CE2 | PHE | B | 503 | 37.788 | 84.421 | 22.384 | 1.00 | 40.11 |
| 2909 | CZ | PHE | B | 503 | 38.850 | 85.311 | 22.391 | 1.00 | 37.06 |
| 2910 | C | PHE | B | 503 | 32.775 | 87.678 | 22.933 | 1.00 | 28.77 |
| 2911 | O | PHE | B | 503 | 32.424 | 88.784 | 23.344 | 1.00 | 28.23 |
| 2912 | N | PHE | B | 504 | 31.963 | 86.637 | 22.887 | 1.00 | 24.91 |
| 2913 | CA | PHE | B | 504 | 30.594 | 86.713 | 23.357 | 1.00 | 25.79 |
| 2914 | CB | PHE | B | 504 | 29.594 | 86.956 | 22.214 | 1.00 | 22.02 |
| 2915 | CG | PHE | B | 504 | 29.343 | 85.766 | 21.326 | 1.00 | 30.92 |
| 2916 | CD1 | PHE | B | 504 | 28.300 | 84.878 | 21.601 | 1.00 | 32.77 |
| 2917 | CD2 | PHE | B | 504 | 30.113 | 85.563 | 20.181 | 1.00 | 34.76 |
| 2918 | CE1 | PHE | B | 504 | 28.030 | 83.804 | 20.751 | 1.00 | 29.72 |
| 2919 | CE2 | PHE | B | 504 | 29.852 | 84.493 | 19.326 | 1.00 | 33.59 |
| 2920 | CZ | PHE | B | 504 | 28.801 | 83.613 | 19.620 | 1.00 | 34.51 |
| 2921 | C | PHE | B | 504 | 30.272 | 85.425 | 24.073 | 1.00 | 28.90 |
| 2922 | O | PHE | B | 504 | 30.849 | 84.378 | 23.783 | 1.00 | 26.02 |
| 2923 | N | VAL | B | 505 | 29.348 | 85.529 | 25.020 | 1.00 | 25.50 |
| 2924 | CA | VAL | B | 505 | 28.919 | 84.404 | 25.804 | 1.00 | 25.53 |
| 2925 | CB | VAL | B | 505 | 29.709 | 84.407 | 27.145 | 1.00 | 29.70 |
| 2926 | CG1 | VAL | B | 505 | 29.124 | 85.416 | 28.098 | 1.00 | 25.34 |
| 2927 | CG2 | VAL | B | 505 | 29.763 | 83.037 | 27.735 | 1.00 | 35.22 |
| 2928 | C | VAL | B | 505 | 27.404 | 84.585 | 26.008 | 1.00 | 26.24 |
| 2929 | O | VAL | B | 505 | 26.893 | 85.705 | 26.004 | 1.00 | 22.25 |
| 2930 | N | PHE | B | 506 | 26.687 | 83.475 | 26.150 | 1.00 | 28.62 |
| 2931 | CA | PHE | B | 506 | 25.239 | 83.507 | 26.346 | 1.00 | 25.84 |
| 2932 | CB | PHE | B | 506 | 24.539 | 82.831 | 25.165 | 1.00 | 29.19 |
| 2933 | CG | PHE | B | 506 | 23.053 | 82.811 | 25.273 | 1.00 | 35.82 |
| 2934 | CD1 | PHE | B | 506 | 22.314 | 83.970 | 25.070 | 1.00 | 42.42 |
| 2935 | CD2 | PHE | B | 506 | 22.387 | 81.639 | 25.600 | 1.00 | 42.16 |
| 2936 | CE1 | PHE | B | 506 | 20.929 | 83.956 | 25.190 | 1.00 | 44.85 |
| 2937 | CE2 | PHE | B | 506 | 21.009 | 81.618 | 25.724 | 1.00 | 41.56 |
| 2938 | CZ | PHE | B | 506 | 20.278 | 82.775 | 25.520 | 1.00 | 45.90 |
| 2939 | C | PHE | B | 506 | 24.881 | 82.756 | 27.627 | 1.00 | 23.92 |
| 2940 | O | PHE | B | 506 | 25.405 | 81.676 | 27.875 | 1.00 | 27.54 |
| 2941 | N | SER | B | 507 | 23.982 | 83.314 | 28.423 | 1.00 | 25.96 |
| 2942 | CA | SER | B | 507 | 23.559 | 82.675 | 29.668 | 1.00 | 25.75 |
| 2943 | CB | SER | B | 507 | 24.119 | 83.456 | 30.865 | 1.00 | 24.23 |
| 2944 | OG | SER | B | 507 | 23.643 | 82.926 | 32.088 | 1.00 | 33.35 |
| 2945 | C | SER | B | 507 | 22.026 | 82.573 | 29.736 | 1.00 | 25.49 |
| 2946 | O | SER | B | 507 | 21.316 | 83.530 | 29.452 | 1.00 | 25.61 |
| 2947 | N | ARG | B | 508 | 21.537 | 81.397 | 30.106 | 1.00 | 26.99 |
| 2948 | CA | ARG | B | 508 | 20.103 | 81.125 | 30.193 | 1.00 | 25.78 |
| 2949 | CE | ARG | B | 508 | 19.779 | 79.948 | 29.275 | 1.00 | 24.46 |
| 2950 | CG | ARG | B | 508 | 18.355 | 79.423 | 29.347 | 1.00 | 28.19 |
| 2951 | CD | ARG | B | 508 | 18.251 | 78.191 | 28.462 | 1.00 | 27.68 |
| 2952 | NE | ARG | B | 508 | 16.877 | 77.735 | 28.296 | 1.00 | 38.82 |
| 2953 | CZ | ARG | B | 508 | 16.245 | 76.959 | 29.167 | 1.00 | 40.66 |
| 2954 | NH1 | ARG | B | 508 | 16.874 | 76.554 | 30.260 | 1.00 | 39.61 |
| 2955 | NH2 | ARG | B | 508 | 14.987 | 76.593 | 28.949 | 1.00 | 40.23 |
| 2956 | C | ARG | B | 508 | 19.690 | 80.784 | 31.629 | 1.00 | 24.52 |
| 2957 | O | ARG | B | 508 | 20.366 | 80.006 | 32.290 | 1.00 | 24.88 |
| 2958 | N | LEU | B | 509 | 18.587 | 81.354 | 32.108 | 1.00 | 26.27 |
| 2959 | CA | LEU | B | 509 | 18.110 | 81.088 | 33.478 | 1.00 | 31.33 |
| 2960 | CB | LEU | B | 509 | 18.394 | 82.297 | 34.379 | 1.00 | 32.56 |
| 2961 | CG | LEU | B | 509 | 17.826 | 82.289 | 35.808 | 1.00 | 33.39 |
| 2962 | CD1 | LEU | B | 509 | 18.551 | 81.250 | 36.666 | 1.00 | 27.13 |
| 2963 | CD2 | LEU | B | 509 | 17.986 | 83.679 | 36.409 | 1.00 | 27.35 |
| 2964 | C | LEU | B | 509 | 16.612 | 80.770 | 33.550 | 1.00 | 33.20 |
| 2965 | O | LEU | B | 509 | 15.777 | 81.654 | 33.379 | 1.00 | 35.36 |
| 2966 | N | GLU | B | 510 | 16.270 | 79.509 | 33.814 | 1.00 | 33.43 |
| 2967 | CA | CLD | B | 510 | 14.865 | 79.121 | 33.918 | 1.00 | 37.45 |
| 2968 | CE | CLD | B | 510 | 14.720 | 77.598 | 33.795 | 1.00 | 38.70 |
| 2969 | CG | GLU | B | 510 | 15.248 | 77.052 | 32.471 | 1.00 | 53.61 |
| 2970 | CD | GLU | B | 510 | 14.878 | 75.591 | 32.203 | 1.00 | 62.03 |
| 2971 | OE1 | CLD | B | 510 | 15.161 | 74.723 | 33.058 | 1.00 | 65.31 |
| 2972 | OE2 | CLD | B | 510 | 14.310 | 75.312 | 31.124 | 1.00 | 63.62 |
| 2973 | C | CLD | B | 510 | 14.315 | 79.593 | 35.259 | 1.00 | 37.95 |
| 2974 | O | GLU | B | 510 | 14.965 | 79.413 | 36.292 | 1.00 | 35.84 |
| 2975 | N | VAL | B | 511 | 13.134 | 80.209 | 35.255 | 1.00 | 37.36 |
| 2976 | CA | VAL | B | 511 | 12.552 | 80.697 | 36.504 | 1.00 | 39.69 |
| 2977 | CB | VAL | B | 511 | 12.588 | 82.234 | 36.587 | 1.00 | 29.12 |
| 2978 | CG1 | VAL | B | 511 | 14.010 | 82.725 | 36.411 | 1.00 | 28.52 |
| 2979 | CG2 | VAL | B | 511 | 11.667 | 82.836 | 35.546 | 1.00 | 31.67 |
| 2980 | C | VAL | B | 511 | 11.110 | 80.243 | 36.708 | 1.00 | 45.51 |
| 2981 | O | VAL | B | 511 | 10.444 | 79.824 | 35.756 | 1.00 | 46.84 |
| 2982 | N | THR | B | 512 | 10.636 | 80.331 | 37.950 | 1.00 | 47.32 |
| 2983 | CA | THR | B | 512 | 9.274 | 79.927 | 38.287 | 1.00 | 51.20 |
| 2984 | CE | THR | B | 512 | 9.129 | 79.678 | 39.796 | 1.00 | 49.91 |
| 2985 | OG1 | THR | B | 512 | 9.458 | 80.877 | 40.508 | 1.00 | 46.57 |
| 2986 | CG2 | THR | B | 512 | 10.056 | 78.554 | 40.242 | 1.00 | 49.02 |
| 2987 | C | THR | B | 512 | 8.288 | 81.015 | 37.887 | 1.00 | 54.77 |
| 2988 | O | THR | B | 512 | 8.691 | 82.133 | 37.580 | 1.00 | 54.38 |
| 2989 | N | ARG | B | 513 | 6.998 | 80.684 | 37.894 | 1.00 | 56.48 |
| 2990 | CA | ARG | B | 513 | 5.958 | 81.645 | 37.535 | 1.00 | 56.58 |
| 2991 | CE | ARG | B | 513 | 4.574 | 80.991 | 37.597 | 1.00 | 61.58 |
| 2992 | CG | ARG | B | 513 | 3.570 | 81.570 | 36.611 | 1.00 | 67.62 |
| 2993 | CD | ARG | B | 513 | 3.294 | 83.034 | 36.879 | 1.00 | 75.58 |
| 2994 | NE | ARG | B | 513 | 2.863 | 83.743 | 35.678 | 1.00 | 83.06 |
| 2995 | CZ | ARG | B | 513 | 2.519 | 85.028 | 35.652 | 1.00 | 86.37 |
| 2996 | NH1 | ARG | B | 513 | 2.551 | 85.744 | 36.764 | 1.00 | 88.41 |
| 2997 | NH2 | ARG | B | 513 | 2.154 | 85.602 | 34.515 | 1.00 | 87.87 |
| 2998 | C | ARG | B | 513 | 5.997 | 82.817 | 38.500 | 1.00 | 55.15 |
| 2999 | O | ARG | B | 513 | 5.744 | 83.961 | 38.117 | 1.00 | 55.55 |
| 3000 | N | ALA | B | 514 | 6.319 | 82.524 | 39.755 | 1.00 | 55.02 |
| 3001 | CA | ALA | B | 514 | 6.386 | 83.545 | 40.792 | 1.00 | 53.96 |
| 3002 | CB | ALA | B | 514 | 6.628 | 82.885 | 42.139 | 1.00 | 53.56 |
| 3003 | C | ALA | B | 514 | 7.472 | 84.585 | 40.505 | 1.00 | 54.12 |
| 3004 | O | ALA | B | 514 | 7.187 | 85.781 | 40.419 | 1.00 | 54.41 |
| 3005 | N | GLU | B | 515 | 8.716 | 84.136 | 40.364 | 1.00 | 54.63 |
| 3006 | CA | GLU | B | 515 | 9.802 | 85.065 | 40.089 | 1.00 | 54.64 |
| 3007 | CB | GLU | B | 515 | 11.166 | 84.375 | 40.238 | 1.00 | 58.15 |
| 3008 | CG | GLU | B | 515 | 11.208 | 82.932 | 39.769 | 1.00 | 64.48 |
| 3009 | CD | GLU | B | 515 | 12.493 | 82.221 | 40.174 | 1.00 | 65.38 |
| 3010 | OE1 | GLU | B | 515 | 12.957 | 82.417 | 41.316 | 1.00 | 66.84 |
| 3011 | OE2 | GLU | B | 515 | 13.037 | 81.451 | 39.359 | 1.00 | 68.35 |
| 3012 | C | GLU | B | 515 | 9.644 | 85.671 | 38.705 | 1.00 | 54.36 |
| 3013 | O | GLU | B | 515 | 10.240 | 86.703 | 38.406 | 1.00 | 51.06 |
| 3014 | N | TRP | B | 516 | 8.831 | 85.037 | 37.863 | 1.00 | 55.85 |
| 3015 | CA | TRP | B | 516 | 8.585 | 85.557 | 36.524 | 1.00 | 57.10 |
| 3016 | CB | TRP | B | 516 | 7.845 | 84.538 | 35.653 | 1.00 | 58.50 |
| 3017 | CG | TRP | B | 516 | 7.950 | 84.874 | 34.194 | 1.00 | 67.12 |
| 3018 | CD2 | TRP | B | 516 | 6.940 | 85.472 | 33.374 | 1.00 | 68.04 |
| 3019 | CE2 | TRP | B | 516 | 7.509 | 85.685 | 32.101 | 1.00 | 71.07 |
| 3020 | CE3 | TRP | B | 516 | 5.608 | 85.849 | 33.593 | 1.00 | 74.06 |
| 3021 | CG1 | TRP | B | 516 | 9.059 | 84.749 | 33.403 | 1.00 | 67.58 |
| 3022 | NE1 | TRP | B | 516 | 8.803 | 85.236 | 32.146 | 1.00 | 68.75 |
| 3023 | CZ2 | TRP | B | 516 | 6.793 | 86.263 | 31.048 | 1.00 | 75.59 |
| 3024 | CZ3 | TRP | B | 516 | 4.895 | 86.425 | 32.542 | 1.00 | 76.50 |
| 3025 | CH2 | TRP | B | 516 | 5.491 | 86.625 | 31.287 | 1.00 | 76.13 |
| 3026 | C | TRP | B | 516 | 7.746 | 86.828 | 36.648 | 1.00 | 57.68 |
| 3027 | O | TRP | B | 516 | 7.775 | 87.691 | 35.773 | 1.00 | 57.71 |
| 3028 | N | GLU | B | 517 | 6.996 | 86.935 | 37.743 | 1.00 | 58.53 |
| 3029 | CA | GLU | B | 517 | 6.173 | 88.113 | 38.008 | 1.00 | 60.83 |
| 3030 | CB | GLU | B | 517 | 5.219 | 87.842 | 39.179 | 1.00 | 65.68 |
| 3031 | CG | GLU | B | 517 | 3.832 | 87.363 | 38.775 | 1.00 | 77.69 |
| 3032 | CD | GLU | B | 517 | 3.078 | 88.387 | 37.932 | 1.00 | 86.61 |
| 3033 | OE1 | GLU | B | 517 | 3.564 | 89.534 | 37.799 | 1.00 | 92.24 |
| 3034 | OE2 | GLU | B | 517 | 1.995 | 88.050 | 37.404 | 1.00 | 89.43 |
| 3035 | C | GLU | B | 517 | 7.053 | 89.323 | 38.345 | 1.00 | 58.89 |
| 3036 | O | GLU | B | 517 | 6.729 | 90.465 | 38.012 | 1.00 | 56.98 |
| 3037 | N | ALA | B | 518 | 8.170 | 89.063 | 39.016 | 1.00 | 58.33 |
| 3038 | CA | ALA | B | 518 | 9.097 | 90.121 | 39.398 | 1.00 | 54.78 |
| 3039 | CB | ALA | B | 518 | 9.422 | 90.005 | 40.880 | 1.00 | 55.11 |
| 3040 | C | ALA | B | 518 | 10.373 | 90.003 | 38.560 | 1.00 | 53.56 |
| 3041 | O | ALA | B | 518 | 11.480 | 90.225 | 39.055 | 1.00 | 52.54 |
| 3042 | N | LYS | B | 519 | 10.219 | 89.681 | 37.281 | 1.00 | 51.73 |
| 3043 | CA | LYS | B | 519 | 11.383 | 89.508 | 36.429 | 1.00 | 52.62 |
| 3044 | CB | LYS | B | 519 | 10.983 | 88.881 | 35.096 | 1.00 | 52.68 |
| 3045 | CG | LYS | B | 519 | 10.253 | 89.792 | 34.146 | 1.00 | 53.71 |
| 3046 | CD | LYS | B | 519 | 9.696 | 88.969 | 32.997 | 1.00 | 61.43 |
| 3047 | CE | LYS | B | 519 | 8.899 | 89.819 | 32.036 | 1.00 | 65.64 |
| 3048 | NZ | LYS | B | 519 | 8.140 | 88.958 | 31.098 | 1.00 | 74.93 |
| 3049 | C | LYS | B | 519 | 12.160 | 90.786 | 36.185 | 1.00 | 52.55 |
| 3050 | O | LYS | B | 519 | 13.313 | 90.743 | 35.761 | 1.00 | 49.84 |
| 3051 | N | ASP | B | 520 | 11.533 | 91.920 | 36.462 | 1.00 | 54.27 |
| 3052 | CA | ASP | B | 520 | 12.176 | 93.219 | 36.280 | 1.00 | 55.54 |
| 3053 | CB | ASP | B | 520 | 11.146 | 94.337 | 36.431 | 1.00 | 65.40 |
| 3054 | CG | ASP | B | 520 | 9.722 | 93.857 | 36.196 | 1.00 | 75.80 |
| 3055 | OD1 | ASP | B | 520 | 9.426 | 93.383 | 35.076 | 1.00 | 83.22 |
| 3056 | OD2 | ASP | B | 520 | 8.900 | 93.948 | 37.136 | 1.00 | 77.87 |
| 3057 | C | ASP | B | 520 | 13.255 | 93.388 | 37.346 | 1.00 | 53.46 |
| 3058 | O | ASP | B | 520 | 14.100 | 94.281 | 37.257 | 1.00 | 51.37 |
| 3059 | N | GLU | B | 521 | 13.217 | 92.521 | 38.353 | 1.00 | 50.72 |
| 3060 | CA | GLU | B | 521 | 14.178 | 92.584 | 39.442 | 1.00 | 50.66 |
| 3061 | CB | GLU | B | 521 | 13.507 | 92.173 | 40.758 | 1.00 | 54.68 |
| 3062 | CG | GLU | B | 521 | 12.510 | 93.192 | 41.301 | 1.00 | 61.75 |
| 3063 | CD | GLU | B | 521 | 11.663 | 92.639 | 42.430 | 1.00 | 68.36 |
| 3064 | OE1 | GLU | B | 521 | 12.232 | 92.137 | 43.422 | 1.00 | 70.14 |
| 3065 | OE2 | GLU | B | 521 | 10.419 | 92.708 | 42.327 | 1.00 | 75.93 |
| 3066 | C | GLU | B | 521 | 15.418 | 91.731 | 39.215 | 1.00 | 46.52 |
| 3067 | O | GLU | B | 521 | 16.392 | 91.857 | 39.952 | 1.00 | 47.21 |
| 3068 | N | PHE | B | 522 | 15.386 | 90.866 | 38.207 | 1.00 | 42.97 |
| 3069 | CA | PHE | B | 522 | 16.531 | 90.011 | 37.919 | 1.00 | 38.46 |
| 3070 | CB | PHE | B | 522 | 16.091 | 88.752 | 37.185 | 1.00 | 37.28 |
| 3071 | CG | PHE | B | 522 | 15.420 | 87.754 | 38.076 | 1.00 | 42.52 |
| 3072 | CD1 | PHE | B | 522 | 14.090 | 87.925 | 38.454 | 1.00 | 44.12 |
| 3073 | CD2 | PHE | B | 522 | 16.133 | 86.671 | 38.592 | 1.00 | 40.14 |
| 3074 | CE1 | PHE | B | 522 | 13.474 | 87.036 | 39.338 | 1.00 | 48.02 |
| 3075 | CE2 | PHE | B | 522 | 15.528 | 85.775 | 39.477 | 1.00 | 42.77 |
| 3076 | CZ | PHE | B | 522 | 14.192 | 85.960 | 39.852 | 1.00 | 41.32 |
| 3077 | C | PHE | B | 522 | 17.594 | 90.748 | 37.130 | 1.00 | 36.02 |
| 3078 | O | PHE | B | 522 | 17.292 | 91.474 | 36.182 | 1.00 | 37.87 |
| 3079 | N | ILE | B | 523 | 18.842 | 90.558 | 37.539 | 1.00 | 32.13 |
| 3080 | CA | ILE | B | 523 | 19.974 | 91.224 | 36.913 | 1.00 | 31.74 |
| 3081 | CB | ILE | B | 523 | 20.634 | 92.208 | 37.908 | 1.00 | 36.58 |
| 3082 | CG2 | ILE | B | 523 | 21.849 | 92.889 | 37.262 | 1.00 | 38.36 |
| 3083 | CG1 | ILE | B | 523 | 19.600 | 93.233 | 38.386 | 1.00 | 39.38 |
| 3084 | CD1 | ILE | B | 523 | 20.086 | 94.083 | 39.536 | 1.00 | 40.35 |
| 3085 | C | ILE | B | 523 | 21.041 | 90.244 | 36.430 | 1.00 | 30.51 |
| 3086 | O | ILE | B | 523 | 21.306 | 89.231 | 37.066 | 1.00 | 29.34 |
| 3087 | N | CYS | B | 524 | 21.651 | 90.554 | 35.298 | 1.00 | 28.73 |
| 3088 | CA | CYS | B | 524 | 22.702 | 89.721 | 34.749 | 1.00 | 27.60 |
| 3089 | C | CYS | B | 524 | 23.965 | 90.519 | 34.964 | 1.00 | 27.54 |
| 3090 | O | CYS | B | 524 | 24.033 | 91.667 | 34.545 | 1.00 | 28.72 |
| 3091 | CB | CYS | B | 524 | 22.477 | 89.504 | 33.251 | 1.00 | 26.75 |
| 3092 | SG | CYS | B | 524 | 23.823 | 88.601 | 32.411 | 1.00 | 36.31 |
| 3093 | N | ARG | B | 525 | 24.964 | 89.925 | 35.603 | 1.00 | 28.42 |
| 3094 | CA | ARG | B | 525 | 26.200 | 90.634 | 35.874 | 1.00 | 28.35 |
| 3095 | CB | ARG | B | 525 | 26.371 | 90.820 | 37.380 | 1.00 | 34.16 |
| 3096 | CG | ARG | B | 525 | 27.688 | 91.480 | 37.769 | 1.00 | 39.75 |
| 3097 | CD | ARG | B | 525 | 27.604 | 92.092 | 39.163 | 1.00 | 48.43 |
| 3098 | NE | ARG | B | 525 | 27.775 | 91.117 | 40.226 | 1.00 | 52.97 |
| 3099 | CZ | ARG | B | 525 | 27.492 | 91.358 | 41.502 | 1.00 | 58.72 |
| 3100 | NE1 | ARG | B | 525 | 27.020 | 92.546 | 41.867 | 1.00 | 55.56 |
| 3101 | NE2 | ARG | B | 525 | 27.670 | 90.410 | 42.414 | 1.00 | 55.90 |
| 3102 | C | ARG | B | 525 | 27.416 | 89.927 | 35.317 | 1.00 | 28.14 |
| 3103 | O | ARG | B | 525 | 27.545 | 88.711 | 35.409 | 1.00 | 28.95 |
| 3104 | N | ALA | B | 526 | 28.317 | 90.708 | 34.743 | 1.00 | 25.82 |
| 3105 | CA | ALA | B | 526 | 29.518 | 90.160 | 34.170 | 1.00 | 25.90 |
| 3106 | CB | ALA | B | 526 | 29.614 | 90.544 | 32.691 | 1.00 | 28.82 |
| 3107 | C | ALA | B | 526 | 30.697 | 90.729 | 34.931 | 1.00 | 27.38 |
| 3108 | O | ALA | B | 526 | 30.701 | 91.910 | 35.253 | 1.00 | 24.32 |
| 3109 | N | VAL | B | 527 | 31.676 | 89.882 | 35.245 | 1.00 | 24.37 |
| 3110 | CA | VAL | B | 527 | 32.873 | 90.334 | 35.923 | 1.00 | 24.12 |
| 3111 | CB | VAL | B | 527 | 33.166 | 89.546 | 37.201 | 1.00 | 24.30 |
| 3112 | CG1 | VAL | B | 527 | 34.510 | 89.963 | 37.762 | 1.00 | 26.10 |
| 3113 | CG2 | VAL | B | 527 | 32.083 | 89.804 | 38.224 | 1.00 | 30.97 |
| 3114 | C | VAL | B | 527 | 33.954 | 90.077 | 34.902 | 1.00 | 26.78 |
| 3115 | O | VAL | B | 527 | 34.078 | 88.969 | 34.375 | 1.00 | 25.10 |
| 3116 | N | HIS | B | 528 | 34.743 | 91.103 | 34.630 | 1.00 | 26.18 |
| 3117 | CA | HIS | B | 528 | 35.771 | 91.015 | 33.616 | 1.00 | 29.17 |
| 3118 | CB | HIS | B | 528 | 35.110 | 91.187 | 32.221 | 1.00 | 28.69 |
| 3119 | CG | HIS | B | 528 | 36.078 | 91.191 | 31.072 | 1.00 | 30.83 |
| 3120 | CD2 | HIS | B | 528 | 36.939 | 92.144 | 30.639 | 1.00 | 30.29 |
| 3121 | ND1 | HIS | B | 528 | 36.282 | 90.096 | 30.258 | 1.00 | 30.55 |
| 3122 | CE1 | HIS | B | 528 | 37.233 | 90.372 | 29.381 | 1.00 | 28.76 |
| 3123 | NE2 | HIS | B | 528 | 37.650 | 91.608 | 29.593 | 1.00 | 23.82 |
| 3124 | C | HIS | B | 528 | 36.818 | 92.102 | 33.870 | 1.00 | 31.62 |
| 3125 | O | HIS | B | 528 | 36.504 | 93.213 | 34.312 | 1.00 | 30.62 |
| 3126 | N | GLU | B | 529 | 38.061 | 91.766 | 33.557 | 1.00 | 34.89 |
| 3127 | CA | GLU | B | 529 | 39.200 | 92.651 | 33.753 | 1.00 | 41.27 |
| 3128 | CB | GLU | B | 529 | 40.471 | 91.960 | 33.257 | 1.00 | 46.90 |
| 3129 | CG | GLU | B | 529 | 41.692 | 92.879 | 33.228 | 1.00 | 61.52 |
| 3130 | CD | GLU | B | 529 | 42.764 | 92.416 | 32.248 | 1.00 | 68.52 |
| 3131 | OE1 | GLU | B | 529 | 43.740 | 93.167 | 32.036 | 1.00 | 68.05 |
| 3132 | OE2 | GLU | B | 529 | 42.631 | 91.306 | 31.687 | 1.00 | 73.22 |
| 3133 | C | GLU | B | 529 | 39.124 | 94.035 | 33.104 | 1.00 | 42.79 |
| 3134 | O | GLU | B | 529 | 39.531 | 95.022 | 33.719 | 1.00 | 42.08 |
| 3135 | N | ALA | B | 530 | 38.622 | 94.098 | 31.868 | 1.00 | 43.28 |
| 3136 | CA | ALA | B | 530 | 38.539 | 95.350 | 31.113 | 1.00 | 44.89 |
| 3137 | CB | ALA | B | 530 | 38.417 | 95.054 | 29.628 | 1.00 | 42.67 |
| 3138 | C | ALA | B | 530 | 37.458 | 96.337 | 31.518 | 1.00 | 47.12 |
| 3139 | O | ALA | B | 530 | 37.463 | 97.474 | 31.052 | 1.00 | 47.05 |
| 3140 | N | ALA | B | 531 | 36.529 | 95.916 | 32.368 | 1.00 | 50.02 |
| 3141 | CA | ALA | B | 531 | 35.470 | 96.808 | 32.820 | 1.00 | 54.71 |
| 3142 | CB | ALA | B | 531 | 34.367 | 95.998 | 33.479 | 1.00 | 52.38 |
| 3143 | C | ALA | B | 531 | 36.081 | 97.813 | 33.808 | 1.00 | 59.71 |
| 3144 | O | ALA | B | 531 | 35.765 | 97.816 | 34.998 | 1.00 | 57.91 |
| 3145 | N | SER | B | 532 | 36.958 | 98.664 | 33.275 | 1.00 | 66.39 |
| 3146 | CA | SER | B | 532 | 37.701 | 99.689 | 34.022 | 1.00 | 71.61 |
| 3147 | CB | SER | B | 532 | 37.651 | 101.030 | 33.272 | 1.00 | 75.15 |
| 3148 | OG | SER | B | 532 | 36.326 | 101.410 | 32.932 | 1.00 | 81.74 |
| 3149 | C | SER | B | 532 | 37.402 | 99.919 | 35.505 | 1.00 | 73.08 |
| 3150 | O | SER | B | 532 | 38.293 | 99.745 | 36.343 | 1.00 | 75.98 |
| 3151 | N | PRO | B | 533 | 36.167 | 100.321 | 35.864 | 1.00 | 72.21 |
| 3152 | CD | PRO | B | 533 | 35.011 | 100.831 | 35.102 | 1.00 | 72.93 |
| 3153 | CA | PRO | B | 533 | 35.956 | 100.524 | 37.299 | 1.00 | 69.88 |
| 3154 | CB | PRO | B | 533 | 34.983 | 101.687 | 37.325 | 1.00 | 69.60 |
| 3155 | CG | PRO | B | 533 | 34.063 | 101.322 | 36.205 | 1.00 | 71.96 |
| 3156 | C | PRO | B | 533 | 35.388 | 99.283 | 37.987 | 1.00 | 67.12 |
| 3157 | O | PRO | B | 533 | 34.331 | 98.780 | 37.600 | 1.00 | 67.77 |
| 3158 | N | SER | B | 534 | 36.101 | 98.798 | 38.997 | 1.00 | 62.96 |
| 3159 | CA | SER | B | 534 | 35.683 | 97.633 | 39.769 | 1.00 | 56.20 |
| 3160 | CB | SER | B | 534 | 34.463 | 97.991 | 40.621 | 1.00 | 57.74 |
| 3161 | OG | SER | B | 534 | 33.463 | 98.615 | 39.838 | 1.00 | 67.85 |
| 3162 | C | SER | B | 534 | 35.406 | 96.342 | 38.989 | 1.00 | 49.03 |
| 3163 | O | SER | B | 534 | 34.789 | 95.424 | 39.524 | 1.00 | 47.21 |
| 3164 | N | CLN | B | 535 | 35.858 | 96.278 | 37.737 | 1.00 | 42.33 |
| 3165 | CA | GUN | B | 535 | 35.704 | 95.096 | 36.896 | 1.00 | 38.93 |
| 3166 | CB | GLN | B | 535 | 36.713 | 94.012 | 37.316 | 1.00 | 32.30 |
| 3167 | CG | GUN | B | 535 | 38.181 | 94.388 | 37.128 | 1.00 | 35.25 |
| 3168 | CD | GLN | B | 535 | 38.619 | 95.526 | 38.024 | 1.00 | 41.01 |
| 3169 | OE1 | GLN | B | 535 | 38.428 | 95.488 | 39.239 | 1.00 | 43.29 |
| 3170 | NE2 | GLN | B | 535 | 39.220 | 96.545 | 37.431 | 1.00 | 47.79 |
| 3171 | C | GLN | B | 535 | 34.310 | 94.485 | 36.891 | 1.00 | 37.85 |
| 3172 | O | GLN | B | 535 | 34.168 | 93.265 | 36.819 | 1.00 | 36.76 |
| 3173 | N | THR | B | 536 | 33.283 | 95.320 | 36.953 | 1.00 | 37.28 |
| 3174 | CA | THR | B | 536 | 31.925 | 94.805 | 36.955 | 1.00 | 39.00 |
| 3175 | CB | THR | B | 536 | 31.319 | 94.848 | 38.366 | 1.00 | 41.53 |
| 3176 | OG1 | THR | B | 536 | 32.267 | 94.344 | 39.314 | 1.00 | 46.75 |
| 3177 | CG2 | THR | B | 536 | 30.076 | 93.987 | 38.424 | 1.00 | 39.44 |
| 3178 | C | THR | B | 536 | 31.016 | 95.586 | 36.028 | 1.00 | 38.42 |
| 3179 | O | THR | B | 536 | 31.148 | 96.794 | 35.895 | 1.00 | 43.26 |
| 3180 | N | VAL | B | 537 | 30.105 | 94.877 | 35.374 | 1.00 | 37.42 |
| 3181 | CA | VAL | B | 537 | 29.131 | 95.462 | 34.468 | 1.00 | 32.52 |
| 3182 | CB | VAL | B | 537 | 29.619 | 95.466 | 32.990 | 1.00 | 36.29 |
| 3183 | CG1 | VAL | B | 537 | 28.580 | 96.148 | 32.114 | 1.00 | 35.71 |
| 3184 | CG2 | VAL | B | 537 | 30.931 | 96.205 | 32.859 | 1.00 | 43.42 |
| 3185 | C | VAL | B | 537 | 27.876 | 94.594 | 34.562 | 1.00 | 31.41 |
| 3186 | O | VAL | B | 537 | 27.946 | 93.369 | 34.435 | 1.00 | 31.67 |
| 3187 | N | GLN | B | 538 | 26.726 | 95.219 | 34.787 | 1.00 | 27.91 |
| 3188 | CA | GLN | B | 538 | 25.496 | 94.452 | 34.917 | 1.00 | 29.41 |
| 3189 | CB | GLN | B | 538 | 25.231 | 94.136 | 36.392 | 1.00 | 29.17 |
| 3190 | CG | GLN | B | 538 | 24.965 | 95.363 | 37.251 | 1.00 | 29.81 |
| 3191 | CD | GLN | B | 538 | 24.710 | 94.983 | 38.691 | 1.00 | 29.58 |
| 3192 | OE1 | GLN | B | 538 | 25.443 | 94.176 | 39.267 | 1.00 | 37.40 |
| 3193 | NE2 | GLN | B | 538 | 23.676 | 95.559 | 39.285 | 1.00 | 32.13 |
| 3194 | C | GLN | B | 538 | 24.304 | 95.193 | 34.356 | 1.00 | 31.40 |
| 3195 | O | CLN | B | 538 | 24.314 | 96.411 | 34.256 | 1.00 | 33.62 |
| 3196 | N | ARG | B | 539 | 23.267 | 94.449 | 34.002 | 1.00 | 31.64 |
| 3197 | CA | ARG | B | 539 | 22.066 | 95.063 | 33.481 | 1.00 | 34.86 |
| 3198 | CB | ARG | B | 539 | 22.120 | 95.120 | 31.955 | 1.00 | 41.64 |
| 3199 | CG | ARG | B | 539 | 21.123 | 96.078 | 31.338 | 1.00 | 48.95 |
| 3200 | CD | ARG | B | 539 | 21.796 | 96.845 | 30.216 | 1.00 | 59.63 |
| 3201 | NE | ARG | B | 539 | 23.067 | 97.395 | 30.680 | 1.00 | 74.02 |
| 3202 | CZ | ARG | B | 539 | 23.909 | 98.094 | 29.927 | 1.00 | 82.05 |
| 3203 | NH1 | ARG | B | 539 | 23.623 | 98.341 | 28.653 | 1.00 | 87.03 |
| 3204 | NH2 | ARG | B | 539 | 25.040 | 98.547 | 30.454 | 1.00 | 85.40 |
| 3205 | C | ARG | B | 539 | 20.840 | 94.289 | 33.922 | 1.00 | 34.74 |
| 3206 | O | ARG | B | 539 | 20.833 | 93.059 | 33.910 | 1.00 | 37.29 |
| 3207 | N | ALA | B | 540 | 19.806 | 95.020 | 34.328 | 1.00 | 34.85 |
| 3208 | CA | ALA | B | 540 | 18.561 | 94.408 | 34.756 | 1.00 | 34.24 |
| 3209 | CB | ALA | B | 540 | 17.709 | 95.425 | 35.472 | 1.00 | 35.02 |
| 3210 | C | ALA | B | 540 | 17.846 | 93.900 | 33.513 | 1.00 | 35.51 |
| 3211 | O | ALA | B | 540 | 18.082 | 94.395 | 32.405 | 1.00 | 30.77 |
| 3212 | N | VAL | B | 541 | 16.996 | 92.896 | 33.692 | 1.00 | 36.19 |
| 3213 | CA | VAL | B | 541 | 16.251 | 92.326 | 32.580 | 1.00 | 42.85 |
| 3214 | CB | VAL | B | 541 | 15.335 | 91.172 | 33.062 | 1.00 | 43.71 |
| 3215 | CG1 | VAL | B | 541 | 14.284 | 90.856 | 32.016 | 1.00 | 48.75 |
| 3216 | CG2 | VAL | B | 541 | 16.167 | 89.935 | 33.351 | 1.00 | 43.59 |
| 3217 | C | VAL | B | 541 | 15.406 | 93.419 | 31.922 | 1.00 | 48.00 |
| 3218 | O | VAL | B | 541 | 14.537 | 94.013 | 32.564 | 1.00 | 44.96 |
| 3219 | N | SER | B | 542 | 15.673 | 93.694 | 30.649 | 1.00 | 53.41 |
| 3220 | CA | SER | B | 542 | 14.914 | 94.717 | 29.948 | 1.00 | 61.94 |
| 3221 | CB | SER | B | 542 | 15.564 | 95.068 | 28.606 | 1.00 | 62.96 |
| 3222 | OG | SER | B | 542 | 15.462 | 94.004 | 27.679 | 1.00 | 69.91 |
| 3223 | C | SER | B | 542 | 13.517 | 94.163 | 29.728 | 1.00 | 65.98 |
| 3224 | O | SER | B | 542 | 13.347 | 92.972 | 29.466 | 1.00 | 67.12 |
| 3225 | N | VAL | B | 543 | 12.520 | 95.029 | 29.851 | 1.00 | 70.21 |
| 3226 | CA | VAL | B | 543 | 11.134 | 94.625 | 29.679 | 1.00 | 74.63 |
| 3227 | CB | VAL | B | 543 | 10.479 | 94.300 | 31.042 | 1.00 | 78.23 |
| 3228 | CG1 | VAL | B | 543 | 9.026 | 93.887 | 30.840 | 1.00 | 79.65 |
| 3229 | CG2 | VAL | B | 543 | 11.256 | 93.191 | 31.741 | 1.00 | 79.80 |
| 3230 | C | VAL | B | 543 | 10.341 | 95.729 | 28.989 | 1.00 | 75.67 |
| 3231 | O | VAL | B | 543 | 9.847 | 96.626 | 29.704 | 1.00 | 77.58 |
| 3232 | OXT | VAL | B | 543 | 10.240 | 95.693 | 27.744 | 1.00 | 74.95 |
| 3233 | C1 | NAG | B | 2 | 34.354 | 65.734 | 31.802 | 1.00 | 97.60 |
| 3234 | O1 | NAG | B | 2 | 33.245 | 65.687 | 30.975 | 1.00 | 99.81 |
| 3235 | C2 | NAG | B | 2 | 34.505 | 67.148 | 32.413 | 1.00 | 96.75 |
| 3236 | N2 | NAG | B | 2 | 33.295 | 67.525 | 33.111 | 1.00 | 98.55 |
| 3237 | C7 | NAG | B | 2 | 33.264 | 67.560 | 34.440 | 1.00 | 101.24 |
| 3238 | O7 | NAG | B | 2 | 34.215 | 67.219 | 35.144 | 1.00 | 100.19 |
| 3239 | C8 | NAG | B | 2 | 31.977 | 68.033 | 35.094 | 1.00 | 100.76 |
| 3240 | C3 | NAG | B | 2 | 34.781 | 68.206 | 31.328 | 1.00 | 96.21 |
| 3241 | O3 | NAG | B | 2 | 35.075 | 69.462 | 31.938 | 1.00 | 95.30 |
| 3242 | C4 | NAG | B | 2 | 35.966 | 67.760 | 30.458 | 1.00 | 94.75 |
| 3243 | O4 | NAG | B | 2 | 36.156 | 68.676 | 29.375 | 1.00 | 91.92 |
| 3244 | C5 | NAG | B | 2 | 3S.704 | 66.327 | 29.921 | 1.00 | 95.52 |
| 324S | O5 | NAG | B | 2 | 35.S07 | 6S.396 | 31.017 | 1.00 | 96.34 |
| 3246 | C6 | NAG | B | 2 | 36.873 | 6S.82S | 29.101 | 1.00 | 96.13 |
| 3247 | O6 | NAG | B | 2 | 38.093 | 65.877 | 29.834 | 1.00 | 99.34 |
| 3248 | OH2 | TIP | B | 1 | 28.270 | 80.979 | 34.917 | 1.00 | 50.26 |
| 3249 | OH2 | TIP | B | 2 | 50.798 | 88.311 | 45.038 | 1.00 | 49.71 |
| 3250 | OH2 | TIP | B | 3 | 16.542 | 94.946 | 50.298 | 1.00 | 62.73 |
| 3251 | OH2 | TIP | B | 4 | 24.725 | 83.256 | 39.944 | 1.00 | 44.66 |
| 3252 | OH2 | TIP | B | 5 | 40.269 | 78.063 | 37.8S1 | 1.00 | 53.56 |
| 3253 | OH2 | TIP | B | 6 | 9.197 | 79.408 | 26.351 | 1.00 | 44.98 |
| 3254 | OH2 | TIP | B | 7 | 26.398 | 96.745 | 29.206 | 1.00 | 44.08 |
| 3255 | OH2 | TIP | B | 8 | 38.618 | 89.009 | 32.579 | 1.00 | 40.39 |
| 3256 | OH2 | TIP | B | 9 | 39.672 | 81.166 | 40.426 | 1.00 | 40.49 |
| 3257 | OH2 | TIP | B | 10 | 17.116 | 78.215 | 37.161 | 1.00 | 46.89 |
| 3258 | OH2 | TIP | B | 11 | 18.243 | 77.296 | 33.917 | 1.00 | 50.51 |
| 3259 | OH2 | TIP | B | 12 | 27.078 | 76.693 | 33.012 | 1.00 | 50.17 |
| 3260 | OH2 | TIP | B | 13 | 35.090 | 58.030 | 33.714 | 1.00 | 54.70 |
| 3261 | OH2 | TIP | B | 14 | 29.218 | 81.786 | 23.355 | 1.00 | 44.57 |
| 3262 | OH2 | TIP | B | 15 | 43.924 | 82.629 | 46.436 | 1.00 | 50.94 |
| 3263 | OH2 | TIP | B | 16 | 1.538 | 85.984 | 28.824 | 1.00 | 56.21 |
| 3264 | OH2 | TIP | B | 17 | 30.S90 | 75.306 | 16.280 | 1.00 | 59.52 |
| 3265 | OH2 | TIP | B | 18 | 23.180 | 95.510 | 42.272 | 1.00 | 55.58 |
| 3266 | OH2 | TIP | B | 19 | 32.511 | 60.357 | 37.976 | 1.00 | 66.56 |
| 3267 | OH2 | TIP | B | 20 | 34.997 | 58.857 | 36.088 | 1.00 | 63.77 |
| 3268 | OH2 | TIP | B | 21 | 13.039 | 76.542 | 38.487 | 1.00 | 62.34 |
| 3269 | OH2 | TIP | B | 22 | 46.374 | 79.044 | 30.275 | 1.00 | 54.44 |
| 3270 | OH2 | TIP | B | 23 | 50.931 | 79.559 | 44.881 | 1.00 | 52.42 |
| 3271 | OH2 | TIP | B | 24 | 37.386 | 70.634 | 30.326 | 1.00 | 60.14 |
| 3272 | OH2 | TIP | B | 25 | 49.964 | 85.552 | 35.048 | 1.00 | 48.11 |
| 3273 | OH2 | TIP | B | 26 | 19.519 | 76.770 | 31.435 | 1.00 | 45.10 |
| 3274 | OH2 | TIP | B | 27 | 36.515 | 71.442 | 28.12S | 1.00 | 59.32 |
| 3275 | OH2 | TIP | B | 28 | S8.S06 | 76.725 | 41.675 | 1.00 | 53.61 |
| 3276 | OH2 | TIP | B | 29 | 38.094 | 79.042 | 44.064 | 1.00 | 60.13 |
| 3277 | OH2 | TIP | B | 30 | 52.870 | 67.201 | 28.126 | 1.00 | 56.34 |
| 3278 | OH2 | TIP | B | 31 | 33.4S6 | 82.100 | 36.961 | 1.00 | 58.14 |
| 3279 | OH2 | TIP | B | 32 | 40.793 | 70.829 | 43.558 | 1.00 | 50.57 |
| 3280 | OH2 | TIP | B | 33 | 9.876 | 81.332 | 43.156 | 1.00 | 56.48 |
| 3281 | OH2 | TIP | B | 34 | 26.776 | 77.910 | 3S.274 | 1.00 | 60.64 |
| 3282 | OH2 | TIP | B | 35 | 32.082 | S6.279 | 37.927 | 1.00 | 56.7S |
| 3283 | OH2 | TIP | B | 36 | 41.915 | 8S.209 | 33.986 | 1.00 | S2.49 |
| 3284 | OH2 | TIP | B | 37 | 35.082 | 80.384 | 18.510 | 1.00 | 56.17 |
| 3285 | OH2 | TIP | B | 38 | 57.244 | 63.192 | 22.89S | 1.00 | S7.49 |
| 3286 | OH2 | TIP | B | 39 | 6.333 | 76.420 | 30.801 | 1.00 | S9.27 |
| 3287 | OH2 | TIP | B | 40 | 29.685 | 90.602 | 44.591 | 1.00 | SS.17 |
| 3288 | OH2 | TIP | B | 41 | 36.388 | 79.659 | 25.044 | 1.00 | 55.25 |
| 3289 | OH2 | TIP | B | 42 | 19.034 | 94.293 | 29.572 | 1.00 | 50.93 |
| 3290 | OH2 | TIP | B | 43 | 40.676 | 79.023 | 31.687 | 1.00 | 58.05 |
| 3291 | OH2 | TIP | B | 44 | 29.928 | 96.295 | 23.009 | 1.00 | 63.22 |
| 3292 | OH2 | TIP | B | 45 | 14.498 | 74.337 | 37.588 | 1.00 | 59.76 |
| 3293 | OH2 | TIP | B | 46 | 34.047 | 76.484 | 31.577 | 1.00 | 61.09 |
| 3294 | OH2 | TIP | B | 47 | 55.169 | 85.314 | 45.805 | 1.00 | 58.75 |
| 3295 | OH2 | TIP | B | 48 | 48.047 | 66.430 | 43.800 | 1.00 | 59.73 |
| 3296 | OH2 | TIP | B | 49 | 33.940 | 96.758 | 23.804 | 1.00 | 53.77 |
| 3297 | OH2 | TIP | B | 50 | 57.073 | 93.041 | 37.538 | 1.00 | 59.31 |
| 3298 | OH2 | TIP | B | 51 | 29.485 | 77.394 | 32.123 | 1.00 | 56.85 |
| 3299 | OH2 | TIP | B | 52 | 28.540 | 96.775 | 19.917 | 1.00 | 60.05 |
| 3300 | OH2 | TIP | B | 53 | 44.894 | 72.951 | 29.410 | 1.00 | 57.10 |
| 3301 | OH2 | TIP | B | 54 | 45.362 | 55.938 | 33.103 | 1.00 | 60.54 |
| 3302 | OH2 | TIP | B | 55 | 48.139 | 75.237 | 29.289 | 1.00 | 62.47 |
| 3303 | OH2 | TIP | B | 56 | 1.034 | 81.059 | 27.240 | 1.00 | 64.33 |
| 3304 | OH2 | TIP | B | 57 | 35.432 | 94.188 | 20.969 | 1.00 | 52.42 |
| 3305 | OH2 | TIP | B | 58 | 17.286 | 95.575 | 43.504 | 1.00 | 60.20 |
| 3306 | OH2 | TIP | B | 59 | 41.972 | 83.557 | 20.901 | 1.00 | 59.26 |
| 3307 | OH2 | TIP | B | 60 | 29.469 | 59.186 | 40.661 | 1.00 | 62.09 |
| 3308 | OH2 | TIP | B | 61 | 51.530 | 82.833 | 43.577 | 1.00 | 65.16 |
| 3309 | OH2 | TIP | B | 62 | 26.641 | 90.990 | 45.263 | 1.00 | 59.85 |
| 3310 | OH2 | TIP | B | 63 | 46.399 | 87.498 | 46.342 | 1.00 | 56.88 |
| 3311 | OH2 | TIP | B | 64 | 25.335 | 75.984 | 42.835 | 1.00 | 53.06 |
| 3312 | OH2 | TIP | B | 65 | 8.193 | 87.217 | 28.036 | 1.00 | 60.26 |
| 3313 | OH2 | TIP | B | 66 | 28.032 | 88.745 | 44.846 | 1.00 | 59.03 |
| 3314 | OH2 | TIP | B | 67 | 40.713 | 72.843 | 34.794 | 1.00 | 54.95 |
| 3315 | OH2 | TIP | B | 68 | 8.183 | 78.374 | 34.568 | 1.00 | 57.65 |
| 3316 | OH2 | TIP | B | 69 | 38.121 | 88.788 | 17.047 | 1.00 | 59.41 |
| 3317 | OH2 | TIP | B | 70 | 40.671 | 93.547 | 43.325 | 1.00 | 62.27 |
| 3318 | OH2 | TIP | B | 71 | 31.413 | 68.974 | 32.122 | 1.00 | 55.86 |
| 3319 | OH2 | TIP | B | 72 | 51.257 | 72.484 | 46.323 | 1.00 | 59.39 |
| 3320 | OH2 | TIP | B | 73 | 30.484 | 83.899 | 40.314 | 1.00 | 59.25 |
| 3321 | OH2 | TIP | B | 74 | 55.561 | 71.680 | 37.852 | 1.00 | 53.17 |
| 3322 | OH2 | TIP | B | 75 | 53.223 | 94.913 | 42.289 | 1.00 | 65.55 |
| 3323 | OH2 | TIP | B | 76 | 55.467 | 63.809 | 25.190 | 1.00 | 62.05 |
| 3324 | OH2 | TIP | B | 77 | 31.319 | 66.997 | 30.418 | 1.00 | 57.87 |
| 3325 | OH2 | TIP | B | 78 | 29.297 | 94.140 | 18.414 | 1.00 | 58.31 |
| 3326 | OH2 | TIP | B | 79 | 9.882 | 83.094 | 29.532 | 1.00 | 53.97 |
| 3327 | OH2 | TIP | B | 80 | 55.788 | 84.688 | 33.186 | 1.00 | 54.45 |
| 3328 | OH2 | TIP | B | 81 | 14.402 | 84.006 | 43.396 | 1.00 | 60.50 |
| 3329 | OH2 | TIP | B | 82 | 21.401 | 99.644 | 31.527 | 1.00 | 54.25 |
| 3330 | OH2 | TIP | B | 83 | 38.747 | 99.305 | 29.133 | 1.00 | 58.03 |
| 3331 | OH2 | TIP | B | 84 | 36.701 | 91.635 | 49.414 | 1.00 | 61.54 |
| 3332 | OH2 | TIP | B | 85 | 53.184 | 73.221 | 55.411 | 1.00 | 71.84 |
| 3333 | OH2 | TIP | B | 86 | 54.962 | 57.175 | 32.829 | 1.00 | 59.92 |
| 3334 | OH2 | TIP | B | 87 | 37.795 | 94.920 | 41.465 | 1.00 | 55.82 |
| 3335 | OH2 | TIP | B | 88 | 47.047 | 68.720 | 45.430 | 1.00 | 57.31 |
| 3336 | OH2 | TIP | B | 89 | 53.386 | 88.730 | 45.407 | 1.00 | 58.45 |
| 3337 | OH2 | TIP | B | 90 | 12.966 | 80.192 | 43.065 | 1.00 | 58.03 |
| 3338 | OH2 | TIP | B | 91 | 25.222 | 92.505 | 24.772 | 1.00 | 50.36 |
| 3339 | OH2 | TIP | B | 92 | 56.971 | 75.837 | 46.165 | 1.00 | 57.19 |
| 3340 | OH2 | TIP | B | 93 | 52.883 | 57.456 | 41.616 | 1.00 | 62.27 |
| 3341 | OH2 | TIP | B | 94 | 21.647 | 72.884 | 37.586 | 1.00 | 61.49 |
| 3342 | OH2 | TIP | B | 95 | 53.819 | 55.355 | 36.996 | 1.00 | 66.72 |
| 3343 | OH2 | TIP | B | 96 | 1.978 | 86.114 | 32.124 | 1.00 | 62.93 |
| 3344 | OH2 | TIP | B | 97 | 3.673 | 88.829 | 34.679 | 1.00 | 59.08 |
| 3345 | OH2 | TIP | B | 98 | 50.288 | 52.210 | 35.596 | 1.00 | 66.54 |
| 3346 | OH2 | TIP | B | 99 | 53.869 | 54.763 | 33.809 | 1.00 | 64.16 |
| 3347 | OH2 | TIP | B | 100 | 39.796 | 81.619 | 49.309 | 1.00 | 59.09 |
| 3348 | OH2 | TIP | B | 101 | 27.537 | 82.048 | 39.451 | 1.00 | 59.99 |
| 3349 | OH2 | TIP | B | 102 | 36.131 | 103.395 | 30.948 | 1.00 | 63.48 |
| 3350 | OH2 | TIP | B | 103 | 56.043 | 76.714 | 32.065 | 1.00 | 60.94 |
| 3351 | OH2 | TIP | B | 104 | 31.838 | 89.133 | 20.071 | 1.00 | 57.13 |
| 3352 | OH2 | TIP | B | 105 | 49.027 | 92.881 | 29.882 | 1.00 | 67.01 |
| 3353 | OH2 | TIP | B | 106 | 52.660 | 69.955 | 44.183 | 1.00 | 61.12 |
| 3354 | OH2 | TIP | B | 107 | 57.992 | 83.903 | 35.849 | 1.00 | 62.98 |
| 3355 | OH2 | TIP | B | 108 | 35.733 | 75.725 | 27.339 | 1.00 | 63.30 |
| 3356 | OH2 | TIP | B | 109 | 40.575 | 98.578 | 26.825 | 1.00 | 57.78 |
| 3357 | OH2 | TIP | B | 110 | 47.643 | 81.928 | 28.944 | 1.00 | 65.34 |
| 3358 | OH2 | TIP | B | 111 | 5.312 | 87.158 | 27.438 | 1.00 | 56.09 |
| 3359 | OH2 | TIP | B | 112 | 49.841 | 88.326 | 23.196 | 1.00 | 62.75 |
| 3360 | OH2 | TIP | B | 113 | 6.598 | 93.132 | 35.325 | 1.00 | 58.88 |
| 3361 | OH2 | TIP | B | 114 | 27.092 | 100.653 | 31.174 | 1.00 | 63.28 |
| 3362 | OH2 | TIP | B | 115 | 42.024 | 53.749 | 39.435 | 1.00 | 65.61 |
| 3363 | OH2 | TIP | B | 116 | 50.539 | 76.914 | 27.922 | 1.00 | 60.98 |
| 3364 | OH2 | TIP | B | 117 | 47.538 | 92.256 | 33.656 | 1.00 | 60.98 |
| 3365 | OH2 | TIP | B | 118 | 5.166 | 74.252 | 29.209 | 1.00 | 58.25 |
| 3366 | OH2 | TIP | B | 119 | 38.217 | 72.245 | 31.919 | 1.00 | 55.39 |
| 3367 | OH2 | TIP | B | 120 | 32.252 | 55.236 | 35.199 | 1.00 | 56.80 |
| 3368 | OH2 | TIP | B | 121 | 32.670 | 57.598 | 33.803 | 1.00 | 61.15 |
| 3369 | OH2 | TIP | B | 122 | 17.786 | 96.981 | 50.771 | 1.00 | 55.82 |
| 3370 | OH2 | TIP | B | 123 | 40.380 | 77.020 | 40.041 | 1.00 | 45.14 |
| 3371 | OH2 | TIP | B | 124 | 42.038 | 88.110 | 33.725 | 1.00 | 55.49 |
| 3372 | OH2 | TIP | B | 125 | 34.758 | 55.495 | 34.823 | 1.00 | 66.22 |
| 3373 | OH2 | TIP | B | 126 | 37.737 | 77.084 | 41.644 | 1.00 | 57.00 |
| 3374 | OH2 | TIP | B | 127 | 29.699 | 98.301 | 28.668 | 1.00 | 57.60 |
| 3375 | OH2 | TIP | B | 128 | 49.020 | 89.370 | 46.224 | 1.00 | 56.82 |
| 3376 | OH2 | TIP | B | 129 | 52.859 | 75.885 | 25.996 | 1.00 | 62.05 |
| 3377 | OH2 | TIP | B | 130 | 30.010 | 59.209 | 36.597 | 1.00 | 66.31 |
| 3378 | OH2 | TIP | B | 131 | 30.914 | 96.977 | 25.231 | 1.00 | 57.71 |
| 3379 | OH2 | TIP | B | 132 | 39.916 | 79.723 | 47.627 | 1.00 | 61.64 |
| 3380 | OH2 | TIP | B | 133 | 44.108 | 57.405 | 44.718 | 1.00 | 61.06 |
| 3381 | OH2 | TIP | B | 134 | 9.863 | 79.457 | 23.833 | 1.00 | 61.78 |
| 3382 | OH2 | TIP | B | 135 | 38.917 | 86.317 | 18.760 | 1.00 | 57.35 |
| 3383 | OH2 | TIP | B | 136 | 30.862 | 81.856 | 35.179 | 1.00 | 54.03 |
| 3384 | OH2 | TIP | B | 137 | 53.704 | 70.947 | 53.894 | 1.00 | 64.00 |
| 3385 | OH2 | TIP | B | 138 | 40.859 | 81.838 | 22.227 | 1.00 | 65.85 |
| 3386 | OH2 | TIP | B | 139 | 37.816 | 96.128 | 20.409 | 1.00 | 60.91 |
| 3387 | OH2 | TIP | B | 140 | 25.705 | 74.474 | 33.305 | 1.00 | 61.62 |
| 3388 | OH2 | TIP | B | 141 | 30.005 | 54.298 | 36.646 | 1.00 | 62.13 |
| 3389 | OH2 | TIP | B | 142 | 10.624 | 79.976 | 28.177 | 1.00 | 58.14 |
| 3390 | OH2 | TIP | B | 143 | 29.498 | 59.385 | 43.076 | 1.00 | 61.05 |
| 3391 | OH2 | TIP | B | 144 | 40.320 | 90.895 | 16.086 | 1.00 | 55.65 |
| 3392 | OH2 | TIP | B | 145 | 55.724 | 66.531 | 26.778 | 1.00 | 64.64 |
| 3393 | OH2 | TIP | B | 146 | 50.659 | 94.738 | 31.683 | 1.00 | 61.54 |
| 3394 | OH2 | TIP | B | 147 | 3.656 | 85.417 | 24.832 | 1.00 | 59.07 |
| 3395 | OH2 | TIP | B | 148 | 16.265 | 75.893 | 38.242 | 1.00 | 60.79 |
| 3396 | OH2 | TIP | B | 149 | 31.777 | 95.407 | 41.590 | 1.00 | 57.59 |
| 3397 | OH2 | TIP | B | 150 | 54.287 | 76.248 | 53.590 | 1.00 | 63.92 |
| 3398 | OH2 | TIP | B | 151 | 33.491 | 79.601 | 16.793 | 1.00 | 62.67 |
| 3399 | OH2 | TIP | B | 152 | 19.155 | 94.756 | 51.118 | 1.00 | 68.07 |
| 3400 | OH2 | TIP | B | 153 | 35.245 | 75.050 | 34.131 | 1.00 | 61.87 |
| 3401 | OH2 | TIP | B | 154 | 56.335 | 76.647 | 34.751 | 1.00 | 66.95 |
| 3402 | OH2 | TIP | B | 155 | 55.588 | 94.784 | 38.616 | 1.00 | 59.56 |
| 3403 | OH2 | TIP | B | 156 | 36.064 | 78.257 | 16.816 | 1.00 | 59.20 |
| 3404 | OH2 | TIP | B | 157 | 55.289 | 57.324 | 36.670 | 1.00 | 67.18 |
| 3405 | OH2 | TIP | B | 158 | 38.244 | 95.684 | 44.137 | 1.00 | 59.37 |
| 3406 | OH2 | TIP | B | 159 | 1.406 | 80.766 | 24.793 | 1.00 | 65.79 |
| 3407 | OH2 | TIP | B | 160 | 46.599 | 89.854 | 33.493 | 1.00 | 58.89 |
| 3408 | OH2 | TIP | B | 161 | 31.235 | 73.902 | 14.323 | 1.00 | 59.68 |
| 3409 | OH2 | TIP | B | 162 | 49.265 | 83.710 | 48.164 | 1.00 | 63.91 |
| 3410 | OH2 | TIP | B | 163 | 12.493 | 91.314 | 27.537 | 1.00 | 58.95 |
| 3411 | OH2 | TIP | B | 164 | 30.439 | 81.890 | 41.630 | 1.00 | 64.19 |
| 3412 | OH2 | TIP | B | 165 | 33.579 | 81.394 | 39.388 | 1.00 | 55.30 |
| 3413 | OH2 | TIP | B | 166 | 23.628 | 85.041 | 47.763 | 1.00 | 55.04 |
| 3414 | OH2 | TIP | B | 167 | 37.026 | 77.530 | 29.328 | 1.00 | 61.56 |
| 3415 | OH2 | TIP | B | 168 | 42.191 | 47.686 | 40.363 | 1.00 | 63.55 |
| 3416 | OH2 | TIP | B | 169 | 48.389 | 87.260 | 21.330 | 1.00 | 62.46 |
| 3417 | OH2 | TIP | B | 170 | 1.110 | 78.818 | 28.205 | 1.00 | 61.28 |
| 3418 | OH2 | TIP | B | 171 | 30.316 | 77.346 | 15.355 | 1.00 | 59.15 |
| 3419 | OH2 | TIP | B | 172 | 56.892 | 58.723 | 34.712 | 1.00 | 66.69 |
| 3420 | OH2 | TIP | B | 173 | 38.573 | 89.823 | 51.022 | 1.00 | 60.64 |
| 3421 | OH2 | TIP | B | 174 | 55.700 | 73.403 | 29.957 | 1.00 | 64.56 |
| 3422 | OH2 | TIP | B | 175 | 2.131 | 82.160 | 29.752 | 1.00 | 59.77 |
| 3423 | OH2 | TIP | B | 176 | 48.652 | 75.463 | 25.876 | 1.00 | 61.95 |
| 3424 | OH2 | TIP | B | 177 | 13.930 | 79.132 | 45.787 | 1.00 | 63.68 |
| 3425 | OH2 | TIP | B | 178 | 49.048 | 79.772 | 28.026 | 1.00 | 62.71 |
| 3426 | OH2 | TIP | B | 179 | 50.873 | 71.018 | 26.463 | 1.00 | 64.40 |
| 3427 | OH2 | TIP | B | 180 | 0.222 | 87.613 | 28.898 | 1.00 | 64.94 |
| 3428 | OH2 | TIP | B | 181 | 36.716 | 99.478 | 26.710 | 1.00 | 60.70 |
| 3429 | OH2 | TIP | B | 182 | 55.733 | 96.794 | 42.680 | 1.00 | 62.42 |
| 3430 | OH2 | TIP | B | 183 | 32.962 | 78.288 | 30.566 | 1.00 | 65.26 |
| 3431 | OH2 | TIP | B | 184 | 50.072 | 74.531 | 27.875 | 1.00 | 58.49 |
| 3432 | OH2 | TIP | B | 185 | 4.066 | 80.299 | 25.717 | 1.00 | 66.38 |
| 3433 | OH2 | TIP | B | 186 | 51.785 | 82.993 | 46.037 | 1.00 | 66.62 |
| 3434 | OH2 | TIP | B | 187 | 6.687 | 89.453 | 25.800 | 1.00 | 63.72 |
| 3435 | OH2 | TIP | B | 188 | 3.415 | 87.824 | 28.810 | 1.00 | 62.49 |
| 3436 | OH2 | TIP | B | 189 | 58.432 | 73.821 | 42.739 | 1.00 | 63.23 |
| 3437 | OH2 | TIP | B | 190 | 3.604 | 89.517 | 32.202 | 1.00 | 65.15 |
| 3438 | OH2 | TIP | B | 191 | 36.662 | 69.392 | 26.901 | 1.00 | 63.27 |
| 3439 | OH2 | TIP | B | 192 | 51.020 | 69.014 | 46.306 | 1.00 | 61.81 |
| 3440 | OH2 | TIP | B | 193 | 34.827 | 72.790 | 27.053 | 1.00 | 59.78 |
| 3441 | OH2 | TIP | B | 194 | 38.979 | 97.644 | 41.760 | 1.00 | 60.91 |
| 3442 | OH2 | TIP | B | 195 | 25.438 | 89.346 | 46.679 | 1.00 | 61.51 |

Another embodiment of the present invention is a 3-D model of a Fc-Cε3/Cε4 region that substantially represents the atomic coordinates specified (i.e., listed) in Table 2.

TABLE 2
Atomic coordinates of 1FP5_dimer.pdb

| ATOM | ATOM | | | | | | | | |
|---|---|---|---|---|---|---|---|---|---|
| # | TYPE | RES | CHN | # | X | Y | Z | OCC | B |
| 1 | N | VAL | A | 336 | 46.157 | 62.618 | 17.991 | 1.00 | 58.93 |
| 2 | CA | VAL | A | 336 | 45.400 | 61.812 | 16.993 | 1.00 | 60.44 |
| 3 | C | VAL | A | 336 | 44.013 | 61.427 | 17.501 | 1.00 | 60.07 |
| 4 | O | VAL | A | 336 | 43.847 | 60.389 | 18.142 | 1.00 | 61.48 |
| 5 | CB | VAL | A | 336 | 46.155 | 60.521 | 16.647 | 1.00 | 60.81 |
| 6 | CG1 | VAL | A | 336 | 45.464 | 59.806 | 15.500 | 1.00 | 61.71 |
| 7 | CG2 | VAL | A | 336 | 47.590 | 60.845 | 16.302 | 1.00 | 64.73 |
| 8 | N | SER | A | 337 | 43.017 | 62.257 | 17.209 | 1.00 | 57.95 |
| 9 | CA | SER | A | 337 | 41.655 | 61.983 | 17.648 | 1.00 | 56.60 |
| 10 | C | SER | A | 337 | 40.683 | 61.842 | 16.476 | 1.00 | 55.17 |
| 11 | O | SER | A | 337 | 40.981 | 62.262 | 15.352 | 1.00 | 54.55 |
| 12 | CB | SER | A | 337 | 41.185 | 63.078 | 18.603 | 1.00 | 57.92 |
| 13 | OG | SER | A | 337 | 41.489 | 64.356 | 18.087 | 1.00 | 64.70 |
| 14 | N | ALA | A | 338 | 39.527 | 61.238 | 16.743 | 1.00 | 52.40 |
| 15 | CA | ALA | A | 338 | 38.522 | 61.010 | 15.711 | 1.00 | 50.69 |
| 16 | C | ALA | A | 338 | 37.109 | 61.300 | 16.192 | 1.00 | 50.25 |
| 17 | O | ALA | A | 338 | 36.772 | 61.062 | 17.354 | 1.00 | 51.17 |
| 18 | CB | ALA | A | 338 | 38.611 | 59.573 | 15.211 | 1.00 | 50.21 |
| 19 | N | TYR | A | 339 | 36.281 | 61.808 | 15.284 | 1.00 | 48.22 |
| 20 | CA | TYR | A | 339 | 34.899 | 62.139 | 15.605 | 1.00 | 46.82 |
| 21 | C | TYR | A | 339 | 33.990 | 61.766 | 14.431 | 1.00 | 44.51 |
| 22 | O | TYR | A | 339 | 34.372 | 61.889 | 13.268 | 1.00 | 42.27 |
| 23 | CB | TYR | A | 339 | 34.765 | 63.638 | 15.915 | 1.00 | 50.52 |
| 24 | CG | TYR | A | 339 | 35.869 | 64.198 | 16.793 | 1.00 | 58.40 |
| 25 | CD1 | TYR | A | 339 | 37.144 | 64.445 | 16.274 | 1.00 | 61.23 |
| 26 | CD2 | TYR | A | 339 | 35.648 | 64.456 | 18.151 | 1.00 | 60.99 |
| 27 | CE1 | TYR | A | 339 | 38.174 | 64.929 | 17.081 | 1.00 | 62.71 |
| 28 | CE2 | TYR | A | 339 | 36.674 | 64.943 | 18.969 | 1.00 | 63.53 |
| 29 | CZ | TYR | A | 339 | 37.933 | 65.176 | 18.427 | 1.00 | 64.33 |
| 30 | OH | TYR | A | 339 | 38.953 | 65.644 | 19.230 | 1.00 | 65.07 |
| 31 | N | LEU | A | 340 | 32.793 | 61.291 | 14.746 | 1.00 | 41.90 |
| 32 | CA | LEU | A | 340 | 31.831 | 60.905 | 13.728 | 1.00 | 41.06 |
| 33 | C | LEU | A | 340 | 30.571 | 61.671 | 14.078 | 1.00 | 41.10 |
| 34 | O | LEU | A | 340 | 30.136 | 61.647 | 15.224 | 1.00 | 43.45 |
| 35 | CB | LEU | A | 340 | 31.583 | 59.392 | 13.779 | 1.00 | 36.93 |
| 36 | CG | LEU | A | 340 | 30.689 | 58.770 | 12.701 | 1.00 | 35.91 |
| 37 | CD1 | LEU | A | 340 | 31.229 | 59.100 | 11.314 | 1.00 | 34.60 |
| 38 | CD2 | LEU | A | 340 | 30.621 | 57.264 | 12.906 | 1.00 | 35.51 |
| 39 | N | SER | A | 341 | 29.990 | 62.368 | 13.108 | 1.00 | 39.82 |
| 40 | CA | SER | A | 341 | 28.790 | 63.152 | 13.385 | 1.00 | 39.85 |
| 41 | C | SER | A | 341 | 27.598 | 62.645 | 12.614 | 1.00 | 37.88 |
| 42 | O | SER | A | 341 | 27.737 | 62.076 | 11.544 | 1.00 | 42.10 |
| 43 | CB | SER | A | 341 | 29.025 | 64.627 | 13.042 | 1.00 | 39.34 |
| 44 | OG | SER | A | 341 | 29.347 | 64.792 | 11.672 | 1.00 | 44.58 |
| 45 | N | ARG | A | 342 | 26.415 | 62.868 | 13.158 | 1.00 | 36.98 |
| 46 | CA | ARG | A | 342 | 25.187 | 62.437 | 12.517 | 1.00 | 35.20 |
| 47 | C | ARG | A | 342 | 24.853 | 63.417 | 11.393 | 1.00 | 33.18 |
| 48 | O | ARG | A | 342 | 25.508 | 64.443 | 11.252 | 1.00 | 33.16 |
| 49 | CB | ARC | A | 342 | 24.070 | 62.394 | 13.566 | 1.00 | 38.57 |
| 50 | CG | ARG | A | 342 | 24.321 | 61.381 | 14.689 | 1.00 | 39.10 |
| 51 | CD | ARG | A | 342 | 23.191 | 61.364 | 15.712 | 1.00 | 43.21 |
| 52 | NE | ARG | A | 342 | 23.231 | 62.544 | 16.570 | 1.00 | 45.40 |
| 53 | CZ | ARG | A | 342 | 24.086 | 62.714 | 17.573 | 1.00 | 48.22 |
| 54 | NE1 | ARG | A | 342 | 24.977 | 61.777 | 17.860 | 1.00 | 52.78 |
| 55 | NH2 | ARG | A | 342 | 24.059 | 63.831 | 18.286 | 1.00 | 54.34 |
| 56 | N | PRO | A | 343 | 23.843 | 63.112 | 10.570 | 1.00 | 33.03 |
| 57 | CA | PRO | A | 343 | 23.497 | 64.040 | 9.481 | 1.00 | 34.14 |
| 58 | C | PRO | A | 343 | 22.907 | 65.339 | 10.035 | 1.00 | 34.06 |
| 59 | O | PRO | A | 343 | 22.302 | 65.341 | 11.106 | 1.00 | 35.44 |
| 60 | CB | PRO | A | 343 | 22.448 | 63.266 | 8.667 | 1.00 | 33.07 |
| 61 | CG | PRO | A | 343 | 22.700 | 61.811 | 9.025 | 1.00 | 35.06 |
| 62 | CD | PRO | A | 343 | 23.029 | 61.885 | 10.499 | 1.00 | 33.27 |
| 63 | N | SER | A | 344 | 23.080 | 66.445 | 9.325 | 1.00 | 32.17 |
| 64 | CA | SER | A | 344 | 22.490 | 67.691 | 9.792 | 1.00 | 31.75 |
| 65 | C | SER | A | 344 | 21.014 | 67.617 | 9.414 | 1.00 | 32.07 |
| 66 | O | SER | A | 344 | 20.660 | 67.115 | 8.344 | 1.00 | 30.21 |
| 67 | CB | SER | A | 344 | 23.144 | 68.907 | 9.118 | 1.00 | 32.20 |
| 68 | OG | SER | A | 344 | 22.665 | 69.113 | 7.799 | 1.00 | 32.50 |
| 69 | N | PRO | A | 345 | 20.127 | 68.088 | 10.300 | 1.00 | 32.31 |
| 70 | CA | PRO | A | 345 | 18.701 | 68.039 | 9.975 | 1.00 | 30.84 |
| 71 | C | PRO | A | 345 | 18.381 | 68.710 | 8.629 | 1.00 | 29.64 |
| 72 | O | PRO | A | 345 | 17.506 | 68.256 | 7.891 | 1.00 | 30.14 |
| 73 | CB | PRO | A | 345 | 18.055 | 68.741 | 11.174 | 1.00 | 30.96 |
| 74 | CG | PRO | A | 345 | 18.941 | 68.301 | 12.307 | 1.00 | 33.18 |
| 75 | CD | PRO | A | 345 | 20.339 | 68.457 | 11.713 | 1.00 | 33.65 |
| 76 | N | PHE | A | 346 | 19.103 | 69.769 | 8.292 | 1.00 | 28.34 |
| 77 | CA | PHE | A | 346 | 18.844 | 70.447 | 7.034 | 1.00 | 31.46 |
| 78 | C | PHE | A | 346 | 19.085 | 69.522 | 5.831 | 1.00 | 32.46 |
| 79 | O | PHE | A | 346 | 18.269 | 69.465 | 4.907 | 1.00 | 32.59 |
| 80 | CB | PHE | A | 346 | 19.711 | 71.706 | 6.916 | 1.00 | 32.72 |
| 81 | CG | PHE | A | 346 | 19.613 | 72.382 | 5.579 | 1.00 | 36.34 |
| 82 | CD1 | PHE | A | 346 | 18.430 | 72.981 | 5.172 | 1.00 | 40.58 |
| 83 | CD2 | PHE | A | 346 | 20.702 | 72.411 | 4.722 | 1.00 | 36.76 |
| 84 | CE1 | PHE | A | 346 | 18.333 | 73.609 | 3.933 | 1.00 | 38.98 |
| 85 | CE2 | PHE | A | 346 | 20.615 | 73.037 | 3.482 | 1.00 | 40.15 |
| 86 | CZ | PHE | A | 346 | 19.425 | 73.637 | 3.086 | 1.00 | 38.60 |
| 87 | N | ASP | A | 347 | 20.203 | 68.797 | 5.850 | 1.00 | 32.92 |
| 88 | CA | ASP | A | 347 | 20.534 | 67.877 | 4.764 | 1.00 | 33.58 |
| 89 | C | ASP | A | 347 | 19.555 | 66.704 | 4.733 | 1.00 | 34.26 |
| 90 | O | ASP | A | 347 | 19.192 | 66.212 | 3.666 | 1.00 | 32.29 |
| 91 | CB | ASP | A | 347 | 21.967 | 67.349 | 4.921 | 1.00 | 28.72 |
| 92 | CG | ASP | A | 347 | 23.008 | 68.364 | 4.521 | 1.00 | 39.62 |
| 93 | OD1 | ASP | A | 347 | 24.225 | 68.085 | 4.671 | 1.00 | 42.08 |
| 94 | OD2 | ASP | A | 347 | 22.609 | 69.449 | 4.046 | 1.00 | 46.23 |
| 95 | N | LEU | A | 348 | 19.120 | 66.276 | 5.911 | 1.00 | 34.31 |
| 96 | CA | LEU | A | 348 | 18.197 | 65.159 | 6.036 | 1.00 | 37.70 |
| 97 | C | LEU | A | 348 | 16.742 | 65.441 | 5.629 | 1.00 | 38.98 |
| 98 | O | LEU | A | 348 | 16.137 | 64.651 | 4.902 | 1.00 | 38.11 |
| 99 | CB | LEU | A | 348 | 18.220 | 64.641 | 7.482 | 1.00 | 40.66 |
| 100 | CG | LEU | A | 348 | 17.333 | 63.441 | 7.840 | 1.00 | 41.99 |
| 101 | CO1 | LEU | A | 348 | 17.738 | 62.212 | 7.005 | 1.00 | 42.77 |
| 102 | CD2 | LEU | A | 348 | 17.476 | 63.131 | 9.341 | 1.00 | 41.00 |
| 103 | N | PHE | A | 349 | 16.183 | 66.560 | 6.083 | 1.00 | 39.68 |
| 104 | CA | PHE | A | 349 | 14.781 | 66.875 | 5.794 | 1.00 | 42.09 |
| 105 | C | PHE | A | 349 | 14.466 | 67.883 | 4.695 | 1.00 | 45.09 |
| 106 | O | PHE | A | 349 | 13.431 | 67.779 | 4.038 | 1.00 | 48.27 |
| 107 | CB | PHE | A | 349 | 14.074 | 67.328 | 7.071 | 1.00 | 37.36 |
| 108 | CG | PHE | A | 349 | 14.189 | 66.356 | 8.200 | 1.00 | 36.93 |
| 109 | CD1 | PHE | A | 349 | 15.021 | 66.624 | 9.282 | 1.00 | 38.16 |
| 110 | CD2 | PHE | A | 349 | 13.487 | 65.156 | 8.174 | 1.00 | 38.04 |
| 111 | CE1 | PHE | A | 349 | 15.155 | 65.715 | 10.319 | 1.00 | 38.11 |
| 112 | CE2 | PHE | A | 349 | 13.614 | 64.231 | 9.213 | 1.00 | 37.58 |
| 113 | CZ | PHE | A | 349 | 14.449 | 64.511 | 10.283 | 1.00 | 40.39 |
| 114 | N | ILE | A | 350 | 15.323 | 68.874 | 4.500 | 1.00 | 46.66 |
| 115 | CA | ILE | A | 350 | 15.052 | 69.854 | 3.461 | 1.00 | 48.94 |
| 116 | C | ILE | A | 350 | 15.695 | 69.400 | 2.150 | 1.00 | 51.15 |
| 117 | O | ILE | A | 350 | 15.007 | 69.095 | 1.179 | 1.00 | 50.51 |
| 118 | CB | ILE | A | 350 | 15.592 | 71.241 | 3.860 | 1.00 | 48.52 |
| 119 | CG1 | ILE | A | 350 | 15.172 | 71.562 | 5.299 | 1.00 | 49.25 |
| 120 | CG2 | ILE | A | 350 | 15.052 | 72.300 | 2.923 | 1.00 | 49.68 |
| 121 | CD1 | ILE | A | 350 | 13.689 | 71.358 | 5.567 | 1.00 | 46.80 |
| 122 | N | ARG | A | 351 | 17.021 | 69.335 | 2.155 | 1.00 | 52.64 |
| 123 | CA | ARG | A | 351 | 17.807 | 68.931 | 1.000 | 1.00 | 53.76 |
| 124 | C | ARG | A | 351 | 17.510 | 67.486 | 0.581 | 1.00 | 53.61 |
| 125 | O | ARG | A | 351 | 17.506 | 67.164 | 0.603 | 1.00 | 53.55 |
| 126 | CB | ARG | A | 351 | 19.287 | 69.095 | 1.341 | 1.00 | 56.64 |
| 127 | CG | ARG | A | 351 | 20.166 | 69.584 | 0.206 | 1.00 | 62.18 |
| 128 | CD | ARG | A | 351 | 21.225 | 70.527 | 0.752 | 1.00 | 66.96 |
| 129 | NE | ARG | A | 351 | 22.389 | 70.642 | 0.119 | 1.00 | 71.13 |
| 130 | CZ | ARG | A | 351 | 23.229 | 69.643 | 0.370 | 1.00 | 76.72 |
| 131 | NH1 | ARG | A | 351 | 23.027 | 68.455 | 0.184 | 1.00 | 78.73 |
| 132 | NH2 | ARG | A | 351 | 24.275 | 69.829 | 1.168 | 1.00 | 77.63 |
| 133 | N | LYS | A | 352 | 17.253 | 66.626 | 1.560 | 1.00 | 53.86 |
| 134 | CA | LYS | A | 352 | 16.954 | 65.215 | 1.312 | 1.00 | 54.12 |
| 135 | C | LYS | A | 352 | 18.151 | 64.428 | 0.772 | 1.00 | 52.02 |
| 136 | O | LYS | A | 352 | 17.996 | 63.522 | 0.046 | 1.00 | 51.77 |
| 137 | CB | LYS | A | 352 | 15.775 | 65.075 | 0.347 | 1.00 | 57.77 |
| 138 | CG | LYS | A | 352 | 14.476 | 65.689 | 0.846 | 1.00 | 62.15 |
| 139 | CD | LYS | A | 352 | 13.348 | 65.439 | 0.154 | 1.00 | 71.06 |
| 140 | CE | LYS | A | 352 | 12.098 | 66.249 | 0.171 | 1.00 | 74.25 |
| 141 | NZ | LYS | A | 352 | 12.293 | 67.707 | 0.085 | 1.00 | 78.35 |
| 142 | N | SER | A | 353 | 19.342 | 64.789 | 1.233 | 1.00 | 48.01 |
| 143 | CA | SER | A | 353 | 20.568 | 64.112 | 0.832 | 1.00 | 45.29 |
| 144 | C | SER | A | 353 | 21.544 | 64.154 | 2.016 | 1.00 | 40.67 |
| 145 | O | SER | A | 353 | 22.546 | 64.875 | 1.994 | 1.00 | 40.76 |
| 146 | CB | SER | A | 353 | 21.177 | 64.791 | 0.398 | 1.00 | 47.97 |
| 147 | CG | SER | A | 353 | 21.448 | 66.155 | 0.147 | 1.00 | 56.20 |
| 148 | N | PRO | A | 354 | 21.244 | 63.375 | 3.070 | 1.00 | 36.53 |
| 149 | CA | PRO | A | 354 | 22.004 | 63.239 | 4.319 | 1.00 | 32.68 |
| 150 | C | PRO | A | 354 | 23.399 | 62.660 | 4.139 | 1.00 | 31.88 |
| 151 | O | PRO | A | 354 | 23.615 | 61.787 | 3.302 | 1.00 | 29.76 |
| 152 | CB | PRO | A | 354 | 21.147 | 62.294 | 5.160 | 1.00 | 30.29 |
| 153 | CG | PRO | A | 354 | 19.799 | 62.340 | 4.513 | 1.00 | 41.68 |
| 154 | CD | PRO | A | 354 | 20.106 | 62.445 | 3.065 | 1.00 | 33.29 |
| 155 | N | THR | A | 355 | 24.342 | 63.154 | 4.932 | 1.00 | 30.03 |
| 156 | CA | THR | A | 355 | 25.704 | 62.644 | 4.902 | 1.00 | 29.70 |
| 157 | C | THR | A | 355 | 26.236 | 62.578 | 6.326 | 1.00 | 30.82 |
| 158 | O | THR | A | 355 | 25.773 | 63.298 | 7.213 | 1.00 | 30.67 |
| 159 | CB | THR | A | 355 | 26.669 | 63.558 | 4.136 | 1.00 | 32.14 |
| 160 | OC1 | THR | A | 355 | 26.806 | 64.785 | 4.860 | 1.00 | 29.97 |
| 161 | CG2 | THR | A | 355 | 26.175 | 63.837 | 2.707 | 1.00 | 31.76 |
| 162 | N | ILE | A | 356 | 27.195 | 61.693 | 6.558 | 1.00 | 30.53 |
| 163 | CA | ILE | A | 356 | 27.816 | 61.631 | 7.868 | 1.00 | 31.56 |
| 164 | C | ILE | A | 356 | 29.279 | 61.860 | 7.575 | 1.00 | 31.79 |
| 165 | O | ILE | A | 356 | 29.752 | 61.561 | 6.475 | 1.00 | 30.35 |
| 166 | CB | ILE | A | 356 | 27.584 | 60.285 | 8.583 | 1.00 | 30.05 |
| 167 | CG1 | ILE | A | 356 | 28.101 | 59.133 | 7.729 | 1.00 | 31.32 |
| 168 | CG2 | ILE | A | 356 | 26.092 | 60.131 | 8.890 | 1.00 | 31.27 |
| 169 | CD1 | ILE | A | 356 | 27.843 | 57.772 | 8.337 | 1.00 | 36.89 |
| 170 | N | THR | A | 357 | 29.995 | 62.402 | 8.550 | 1.00 | 33.31 |
| 171 | CA | THR | A | 357 | 31.395 | 62.719 | 8.349 | 1.00 | 33.68 |
| 172 | C | THR | A | 357 | 32.282 | 62.197 | 9.456 | 1.00 | 35.47 |
| 173 | O | THR | A | 357 | 31.993 | 62.372 | 10.641 | 1.00 | 36.65 |
| 174 | CB | THE | A | 357 | 31.589 | 64.265 | 8.245 | 1.00 | 33.81 |
| 175 | OG1 | THR | A | 357 | 30.917 | 64.756 | 7.077 | 1.00 | 33.71 |
| 176 | CG2 | THR | A | 357 | 33.065 | 64.632 | 8.179 | 1.00 | 29.63 |
| 177 | N | CYS | A | 358 | 33.372 | 61.566 | 9.052 | 1.00 | 35.41 |
| 178 | CA | CYS | A | 358 | 34.346 | 61.039 | 9.990 | 1.00 | 38.12 |
| 179 | C | CYS | A | 358 | 35.507 | 62.030 | 9.935 | 1.00 | 37.77 |
| 180 | O | CYS | A | 358 | 36.127 | 62.205 | 8.890 | 1.00 | 36.67 |
| 181 | CB | CYS | A | 358 | 34.811 | 59.655 | 9.532 | 1.00 | 40.37 |
| 182 | SG | CYS | A | 358 | 35.831 | 58.729 | 10.726 | 1.00 | 49.83 |
| 183 | N | LEU | A | 359 | 35.778 | 62.691 | 11.054 | 1.00 | 39.77 |
| 184 | CA | LEU | A | 359 | 36.853 | 63.668 | 11.127 | 1.00 | 42.86 |
| 185 | C | LEU | A | 359 | 38.007 | 63.168 | 11.977 | 1.00 | 46.01 |
| 186 | O | LEU | A | 359 | 37.831 | 62.852 | 13.152 | 1.00 | 47.43 |
| 187 | CB | LEU | A | 359 | 36.335 | 64.988 | 11.711 | 1.00 | 42.74 |
| 188 | CG | LEU | A | 359 | 37.392 | 65.996 | 12.196 | 1.00 | 45.03 |
| 189 | CD1 | LEU | A | 359 | 38.262 | 66.448 | 11.043 | 1.00 | 47.59 |
| 190 | CD2 | LEU | A | 359 | 36.711 | 67.197 | 12.838 | 1.00 | 47.51 |
| 191 | N | VAL | A | 360 | 39.189 | 63.107 | 11.379 | 1.00 | 48.26 |
| 192 | CA | VAL | A | 360 | 40.380 | 62.669 | 12.090 | 1.00 | 52.34 |
| 193 | C | VAL | A | 360 | 41.357 | 63.835 | 12.217 | 1.00 | 55.48 |
| 194 | O | VAL | A | 360 | 41.672 | 64.508 | 11.234 | 1.00 | 55.57 |
| 195 | CB | VAL | A | 360 | 41.073 | 61.500 | 11.353 | 1.00 | 53.36 |
| 196 | CG1 | VAL | A | 360 | 42.360 | 61.119 | 12.071 | 1.00 | 51.62 |
| 197 | CG2 | VAL | A | 360 | 40.133 | 60.301 | 11.284 | 1.00 | 51.77 |
| 198 | N | VAL | A | 361 | 41.827 | 64.080 | 13.435 | 1.00 | 58.92 |
| 199 | CA | VAL | A | 361 | 42.766 | 65.167 | 13.684 | 1.00 | 63.13 |
| 200 | C | VAL | A | 361 | 44.094 | 64.607 | 14.174 | 1.00 | 67.77 |
| 201 | O | VAL | A | 361 | 44.134 | 63.862 | 15.154 | 1.00 | 68.38 |
| 202 | CB | VAL | A | 361 | 42.223 | 66.158 | 14.747 | 1.00 | 60.50 |
| 203 | CG1 | VAL | A | 361 | 43.245 | 67.242 | 15.014 | 1.00 | 56.92 |
| 204 | CG2 | VAL | A | 361 | 40.929 | 66.786 | 14.270 | 1.00 | 56.10 |
| 205 | N | ASP | A | 362 | 45.174 | 64.965 | 13.485 | 1.00 | 72.90 |
| 206 | CA | ASP | A | 362 | 46.515 | 64.502 | 13.841 | 1.00 | 78.30 |
| 207 | C | ASP | A | 362 | 47.356 | 65.696 | 14.284 | 1.00 | 81.68 |
| 208 | O | ASP | A | 362 | 47.891 | 66.432 | 13.452 | 1.00 | 81.72 |
| 209 | CB | ASP | A | 362 | 47.185 | 63.829 | 12.638 | 1.00 | 80.22 |
| 210 | CG | ASP | A | 362 | 48.444 | 63.070 | 13.020 | 1.00 | 84.67 |
| 211 | OD1 | ASP | A | 362 | 49.122 | 63.487 | 13.983 | 1.00 | 87.54 |
| 212 | OD2 | ASP | A | 362 | 48.763 | 62.063 | 12.351 | 1.00 | 87.19 |
| 213 | N | LEU | A | 363 | 47.474 | 65.877 | 15.597 | 1.00 | 85.84 |
| 214 | CA | LEU | A | 363 | 48.234 | 66.986 | 16.165 | 1.00 | 89.95 |
| 215 | C | LEU | A | 363 | 49.677 | 67.038 | 15.672 | 1.00 | 92.20 |
| 216 | O | LEU | A | 363 | 50.214 | 68.117 | 15.432 | 1.00 | 92.55 |
| 217 | CB | LEU | A | 363 | 48.210 | 66.906 | 17.691 | 1.00 | 91.66 |
| 218 | CG | LEU | A | 363 | 46.823 | 66.881 | 18.342 | 1.00 | 92.93 |
| 219 | CD1 | LEU | A | 363 | 46.976 | 66.787 | 19.854 | 1.00 | 95.41 |
| 220 | CD2 | LEU | A | 363 | 46.049 | 68.129 | 17.963 | 1.00 | 92.73 |
| 221 | N | ALA | A | 364 | 50.304 | 65.874 | 15.527 | 1.00 | 94.76 |
| 222 | CA | ALA | A | 364 | 51.684 | 65.799 | 15.049 | 1.00 | 97.66 |
| 223 | C | ALA | A | 364 | 51.769 | 64.206 | 13.890 | 1.00 | 99.72 |
| 224 | O | ALA | A | 364 | 51.604 | 63.602 | 14.079 | 1.00 | 100.44 |
| 225 | CB | ALA | A | 364 | 52.604 | 65.365 | 16.179 | 1.00 | 97.99 |
| 226 | N | PRO | A | 365 | 52.023 | 65.303 | 12.671 | 1.00 | 101.15 |
| 227 | CA | PRO | A | 365 | 52.116 | 64.427 | 11.498 | 1.00 | 102.25 |
| 228 | C | PRO | A | 365 | 53.437 | 63.705 | 11.245 | 1.00 | 103.43 |
| 229 | O | PRO | A | 365 | 54.497 | 64.326 | 11.166 | 1.00 | 104.19 |
| 230 | CB | PRO | A | 365 | 51.771 | 65.363 | 10.339 | 1.00 | 101.91 |
| 231 | CG | PRO | A | 365 | 50.983 | 66.479 | 11.002 | 1.00 | 102.27 |
| 232 | CD | PRO | A | 365 | 51.767 | 66.690 | 12.254 | 1.00 | 101.40 |
| 233 | N | SER | A | 366 | 53.344 | 62.385 | 11.121 | 1.00 | 104.18 |
| 234 | CA | SER | A | 366 | 54.468 | 61.512 | 10.801 | 1.00 | 104.43 |
| 235 | C | SER | A | 366 | 53.924 | 61.027 | 9.463 | 1.00 | 104.05 |
| 236 | O | SER | A | 366 | 52.736 | 60.712 | 9.373 | 1.00 | 104.36 |
| 237 | CB | SER | A | 366 | 54.545 | 60.351 | 11.788 | 1.00 | 105.50 |
| 238 | OG | SER | A | 366 | 53.377 | 59.560 | 11.687 | 1.00 | 106.70 |
| 239 | N | LYS | A | 367 | 54.739 | 60.955 | 8.418 | 1.00 | 103.19 |
| 240 | CA | LYS | A | 367 | 54.138 | 60.559 | 7.156 | 1.00 | 102.08 |
| 241 | C | LYS | A | 367 | 53.970 | 59.109 | 6.733 | 1.00 | 100.74 |
| 242 | O | LYS | A | 367 | 54.917 | 58.366 | 6.460 | 1.00 | 101.05 |
| 243 | CB | LYS | A | 367 | 54.732 | 61.364 | 5.998 | 1.00 | 103.30 |
| 244 | CO | LYS | A | 367 | 56.216 | 61.257 | 5.755 | 1.00 | 105.00 |
| 245 | CD | LYS | A | 367 | 56.554 | 62.226 | 4.633 | 1.00 | 104.80 |
| 246 | CE | LYS | A | 367 | 57.990 | 62.133 | 4.170 | 1.00 | 104.70 |
| 247 | NZ | LYS | A | 367 | 58.214 | 63.076 | 3.034 | 1.00 | 105.18 |
| 248 | N | GLY | A | 368 | 52.691 | 58.761 | 6.678 | 1.00 | 98.70 |
| 249 | CA | GLY | A | 368 | 52.192 | 57.464 | 6.272 | 1.00 | 95.62 |
| 250 | C | GLY | A | 368 | 50.799 | 57.900 | 5.856 | 1.00 | 93.12 |
| 251 | O | GLY | A | 368 | 50.457 | 59.062 | 6.065 | 1.00 | 93.17 |
| 252 | N | THR | A | 369 | 49.983 | 57.031 | 5.277 | 1.00 | 90.15 |
| 253 | CA | THR | A | 369 | 48.652 | 57.475 | 4.880 | 1.00 | 86.41 |
| 254 | C | THR | A | 369 | 47.623 | 57.180 | 5.964 | 1.00 | 83.04 |
| 255 | O | THR | A | 369 | 47.746 | 56.198 | 6.699 | 1.00 | 82.54 |
| 256 | CB | THR | A | 369 | 48.203 | 56.808 | 3.559 | 1.00 | 87.73 |
| 257 | OG1 | THR | A | 369 | 48.178 | 55.383 | 3.721 | 1.00 | 88.60 |
| 258 | CG2 | THR | A | 369 | 49.157 | 57.177 | 2.429 | 1.00 | 86.64 |
| 259 | N | VAL | A | 370 | 46.620 | 58.048 | 6.073 | 1.00 | 79.07 |
| 260 | CA | VAL | A | 370 | 45.559 | 57.871 | 7.058 | 1.00 | 74.46 |
| 261 | C | VAL | A | 370 | 44.339 | 57.325 | 6.332 | 1.00 | 72.21 |
| 262 | O | VAL | A | 370 | 43.762 | 57.998 | 5.482 | 1.00 | 71.35 |
| 263 | CB | VAL | A | 370 | 45.180 | 59.199 | 7.732 | 1.00 | 73.33 |
| 264 | CG1 | VAL | A | 370 | 44.254 | 58.933 | 8.904 | 1.00 | 70.92 |
| 265 | CG2 | VAL | A | 370 | 46.427 | 59.926 | 8.185 | 1.00 | 69.77 |
| 266 | N | ASN | A | 371 | 43.947 | 56.102 | 6.674 | 1.00 | 70.13 |
| 267 | CA | ASN | A | 371 | 42.809 | 55.463 | 6.027 | 1.00 | 68.15 |
| 268 | C | ASN | A | 371 | 41.521 | 55.443 | 6.838 | 1.00 | 65.65 |
| 269 | O | ASN | A | 371 | 41.533 | 55.248 | 8.054 | 1.00 | 64.64 |
| 270 | CB | ASN | A | 371 | 43.179 | 54.036 | 5.631 | 1.00 | 70.12 |
| 271 | CG | ASN | A | 371 | 44.270 | 53.995 | 4.592 | 1.00 | 73.56 |
| 272 | OD1 | ASN | A | 371 | 44.081 | 54.447 | 3.462 | 1.00 | 75.10 |
| 273 | ND2 | ASN | A | 371 | 45.427 | 53.463 | 4.969 | 1.00 | 74.76 |
| 274 | N | LEU | A | 372 | 40.408 | 55.651 | 6.141 | 1.00 | 62.66 |
| 275 | CA | LEU | A | 372 | 39.088 | 55.641 | 6.754 | 1.00 | 59.53 |
| 276 | C | LEU | A | 372 | 38.226 | 54.673 | 5.963 | 1.00 | 56.68 |
| 277 | O | LEU | A | 372 | 38.071 | 54.812 | 4.753 | 1.00 | 56.70 |
| 278 | CB | LEU | A | 372 | 38.459 | 57.040 | 6.728 | 1.00 | 59.44 |
| 279 | CG | LEU | A | 372 | 39.217 | 58.172 | 7.422 | 1.00 | 60.55 |
| 280 | CD1 | LEU | A | 372 | 38.323 | 59.386 | 7.521 | 1.00 | 61.28 |
| 281 | CD2 | LEU | A | 372 | 39.644 | 57.739 | 8.807 | 1.00 | 62.83 |
| 282 | N | THR | A | 373 | 37.669 | 53.689 | 6.654 | 1.00 | 53.76 |
| 283 | CA | THR | A | 373 | 36.834 | 52.685 | 6.015 | 1.00 | 51.64 |
| 284 | C | THR | A | 373 | 35.420 | 52.750 | 6.588 | 1.00 | 48.20 |
| 285 | O | THR | A | 373 | 35.245 | 52.799 | 7.804 | 1.00 | 46.65 |
| 286 | CB | THR | A | 373 | 37.399 | 51.275 | 6.268 | 1.00 | 53.15 |
| 287 | OG1 | THR | A | 373 | 38.811 | 51.276 | 6.027 | 1.00 | 57.77 |
| 288 | CG2 | THR | A | 373 | 36.729 | 50.261 | 5.353 | 1.00 | 53.75 |
| 289 | N | TRP | A | 374 | 34.419 | 52.749 | 5.715 | 1.00 | 45.34 |
| 290 | CA | TRP | A | 374 | 33.025 | 52.801 | 6.158 | 1.00 | 44.94 |
| 291 | C | TRP | A | 374 | 32.341 | 51.439 | 6.059 | 1.00 | 43.68 |
| 292 | O | TRP | A | 374 | 32.636 | 50.655 | 5.162 | 1.00 | 42.67 |
| 293 | CD | TRP | A | 374 | 32.209 | 53.795 | 5.316 | 1.00 | 41.63 |
| 294 | CG | TRP | A | 374 | 32.623 | 55.229 | 5.443 | 1.00 | 41.50 |
| 295 | CD1 | TRP | A | 374 | 33.476 | 55.912 | 4.623 | 1.00 | 39.67 |
| 296 | CD2 | TRP | A | 374 | 32.205 | 56.159 | 6.452 | 1.00 | 35.69 |
| 297 | NE1 | TRP | A | 374 | 33.612 | 57.208 | 5.059 | 1.00 | 35.06 |
| 298 | CE2 | TRP | A | 374 | 32.844 | 57.386 | 6.178 | 1.00 | 34.53 |
| 299 | CE3 | TRP | A | 374 | 31.353 | 56.072 | 7.558 | 1.00 | 32.37 |
| 300 | CZ2 | TRP | A | 374 | 32.657 | 58.521 | 6.971 | 1.00 | 35.43 |
| 301 | CZ3 | TRP | A | 374 | 31.166 | 57.199 | 8.349 | 1.00 | 36.11 |
| 302 | CH2 | TRP | A | 374 | 31.818 | 58.410 | 8.050 | 1.00 | 37.51 |
| 303 | N | SER | A | 375 | 31.421 | 51.170 | 6.981 | 1.00 | 43.53 |
| 304 | CA | SER | A | 375 | 30.666 | 49.922 | 6.960 | 1.00 | 43.57 |
| 305 | C | SER | A | 375 | 29.330 | 50.089 | 7.662 | 1.00 | 42.59 |
| 306 | O | SER | A | 375 | 29.189 | 50.931 | 8.550 | 1.00 | 43.68 |
| 307 | CD | SER | A | 375 | 31.447 | 48.780 | 7.626 | 1.00 | 45.03 |
| 308 | OG | SER | A | 375 | 31.482 | 48.909 | 9.034 | 1.00 | 45.66 |
| 309 | N | ARG | A | 376 | 28.349 | 49.301 | 7.234 | 1.00 | 40.79 |
| 310 | CA | ARG | A | 376 | 27.031 | 49.321 | 7.834 | 1.00 | 40.12 |
| 311 | C | ARG | A | 376 | 26.911 | 48.102 | 8.749 | 1.00 | 40.59 |
| 312 | O | ARG | A | 376 | 27.207 | 46.982 | 8.348 | 1.00 | 40.90 |
| 313 | CB | ARG | A | 376 | 25.947 | 49.255 | 6.766 | 1.00 | 40.03 |
| 314 | CG | ARG | A | 376 | 25.855 | 50.468 | 5.863 | 1.00 | 43.20 |
| 315 | CD | ARG | A | 376 | 24.402 | 50.717 | 5.501 | 1.00 | 41.05 |
| 316 | NE | ARG | A | 376 | 24.120 | 50.429 | 4.108 | 1.00 | 50.71 |
| 317 | CZ | ARG | A | 376 | 22.895 | 50.300 | 3.612 | 1.00 | 53.97 |
| 318 | NH1 | ARG | A | 376 | 21.842 | 50.428 | 4.407 | 1.00 | 51.88 |
| 319 | NH2 | ARG | A | 376 | 22.726 | 50.058 | 2.318 | 1.00 | 55.55 |
| 320 | N | ALA | A | 377 | 26.471 | 48.319 | 9.978 | 1.00 | 40.20 |
| 321 | CA | ALA | A | 377 | 26.327 | 47.218 | 10.919 | 1.00 | 40.25 |
| 322 | C | ALA | A | 377 | 25.470 | 46.100 | 10.314 | 1.00 | 40.38 |
| 323 | O | ALA | A | 377 | 25.621 | 44.943 | 10.678 | 1.00 | 41.16 |
| 324 | CD | ALA | A | 377 | 25.697 | 47.721 | 12.222 | 1.00 | 33.12 |
| 325 | N | SER | A | 378 | 24.585 | 46.456 | 9.386 | 1.00 | 40.21 |
| 326 | CA | SER | A | 378 | 23.697 | 45.491 | 8.746 | 1.00 | 41.95 |
| 327 | C | SER | A | 378 | 24.412 | 44.694 | 7.664 | 1.00 | 44.92 |
| 328 | O | SER | A | 378 | 23.856 | 43.734 | 7.134 | 1.00 | 44.28 |
| 329 | CD | SER | A | 378 | 22.504 | 46.199 | 8.108 | 1.00 | 40.91 |
| 330 | OG | SER | A | 378 | 22.907 | 46.863 | 6.920 | 1.00 | 40.09 |
| 331 | N | GLY | A | 379 | 25.633 | 45.107 | 7.332 | 1.00 | 46.93 |
| 332 | CA | GLY | A | 379 | 26.405 | 44.419 | 6.313 | 1.00 | 49.77 |
| 333 | C | GLY | A | 379 | 26.088 | 44.884 | 4.904 | 1.00 | 50.60 |
| 334 | O | GLY | A | 379 | 26.817 | 44.576 | 3.958 | 1.00 | 52.95 |
| 335 | N | LYS | A | 380 | 24.995 | 45.626 | 4.755 | 1.00 | 53.72 |
| 336 | CA | LYS | A | 380 | 24.612 | 46.122 | 3.443 | 1.00 | 54.13 |
| 337 | C | LYS | A | 380 | 25.689 | 47.036 | 2.845 | 1.00 | 48.16 |
| 338 | O | LYS | A | 380 | 26.592 | 47.513 | 3.544 | 1.00 | 45.65 |
| 339 | CD | LYS | A | 380 | 23.262 | 46.848 | 3.517 | 1.00 | 62.44 |
| 340 | CG | LYS | A | 380 | 22.078 | 45.908 | 3.710 | 1.00 | 67.72 |
| 341 | CD | LYS | A | 380 | 20.748 | 46.625 | 3.550 | 1.00 | 69.32 |
| 342 | CE | LYS | A | 380 | 19.587 | 45.644 | 3.619 | 1.00 | 73.14 |
| 343 | NZ | LYS | A | 380 | 18.267 | 46.320 | 3.467 | 1.00 | 77.37 |
| 344 | N | PRO | A | 381 | 25.614 | 47.277 | 1.529 | 1.00 | 56.07 |
| 345 | CA | PRO | A | 381 | 26.586 | 48.128 | 0.835 | 1.00 | 56.46 |
| 346 | C | PRO | A | 381 | 26.535 | 49.607 | 1.225 | 1.00 | 54.32 |
| 347 | O | PRO | A | 381 | 25.467 | 50.154 | 1.511 | 1.00 | 56.76 |
| 348 | CB | PRO | A | 381 | 26.225 | 47.936 | 0.640 | 1.00 | 60.70 |
| 349 | CG | PRO | A | 381 | 25.481 | 46.622 | 0.667 | 1.00 | 62.01 |
| 350 | CD | PRO | A | 381 | 24.655 | 46.695 | 0.574 | 1.00 | 59.24 |
| 351 | N | VAL | A | 382 | 27.701 | 50.244 | 1.234 | 1.00 | 55.74 |
| 352 | CA | VAL | A | 382 | 27.795 | 51.669 | 1.531 | 1.00 | 55.58 |
| 353 | C | VAL | A | 382 | 28.192 | 52.353 | 0.220 | 1.00 | 55.56 |
| 354 | O | VAL | A | 382 | 28.866 | 51.752 | 0.620 | 1.00 | 55.22 |
| 355 | CE | VAL | A | 382 | 28.874 | 51.980 | 2.607 | 1.00 | 55.39 |
| 356 | CG1 | VAL | A | 382 | 28.592 | 51.196 | 3.878 | 1.00 | 55.94 |
| 357 | CG2 | VAL | A | 382 | 30.261 | 51.664 | 2.075 | 1.00 | 53.06 |
| 358 | N | ASN | A | 383 | 27.773 | 53.600 | 0.045 | 1.00 | 54.38 |
| 359 | CA | ASN | A | 383 | 28.090 | 54.354 | 1.164 | 1.00 | 54.63 |
| 360 | C | ASN | A | 383 | 29.532 | 54.843 | 1.190 | 1.00 | 53.85 |
| 361 | O | ASN | A | 383 | 30.303 | 54.630 | 0.254 | 1.00 | 52.58 |
| 362 | CB | ASN | A | 383 | 27.171 | 55.566 | 1.282 | 1.00 | 59.07 |
| 363 | CG | ASN | A | 383 | 25.717 | 55.186 | 1.356 | 1.00 | 64.60 |
| 364 | OD1 | ASN | A | 383 | 24.836 | 56.024 | 1.169 | 1.00 | 70.14 |
| 365 | ND2 | ASN | A | 383 | 25.450 | 53.915 | 1.638 | 1.00 | 71.07 |
| 366 | N | HIS | A | 384 | 29.890 | 55.506 | 2.281 | 1.00 | 53.46 |
| 367 | CA | HIS | A | 384 | 31.229 | 56.057 | 2.426 | 1.00 | 52.22 |
| 368 | C | HIS | A | 384 | 31.326 | 57.302 | 1.540 | 1.00 | 49.10 |
| 369 | O | HIS | A | 384 | 30.400 | 58.105 | 1.485 | 1.00 | 45.53 |
| 370 | CE | HIS | A | 384 | 31.489 | 56.417 | 3.893 | 1.00 | 59.11 |
| 371 | CG | HIS | A | 384 | 31.676 | 55.223 | 4.778 | 1.00 | 62.16 |
| 372 | ND1 | HIS | A | 384 | 32.738 | 54.357 | 4.639 | 1.00 | 69.15 |
| 373 | CD2 | HIS | A | 384 | 30.931 | 54.745 | 5.803 | 1.00 | 70.28 |
| 374 | CE1 | HIS | A | 384 | 32.640 | 53.394 | 5.539 | 1.00 | 75.01 |
| 375 | NE2 | HIS | A | 384 | 31.553 | 53.605 | 6.258 | 1.00 | 74.58 |
| 376 | N | SER | A | 385 | 32.448 | 57.454 | 0.849 | 1.00 | 45.07 |
| 377 | CA | SER | A | 385 | 32.641 | 58.583 | 0.040 | 1.00 | 44.61 |
| 378 | C | SER | A | 385 | 33.617 | 59.627 | 0.504 | 1.00 | 44.58 |
| 379 | O | SER | A | 385 | 34.380 | 59.356 | 1.421 | 1.00 | 44.21 |
| 380 | CB | SER | A | 385 | 33.113 | 58.076 | 1.409 | 1.00 | 44.61 |
| 381 | OG | SER | A | 385 | 34.293 | 57.290 | 1.311 | 1.00 | 41.13 |
| 382 | N | THR | A | 386 | 33.575 | 60.827 | 0.065 | 1.00 | 44.32 |
| 383 | CA | THR | A | 386 | 34.450 | 61.916 | 0.346 | 1.00 | 44.35 |
| 384 | C | THR | A | 386 | 35.487 | 62.156 | 0.737 | 1.00 | 44.09 |
| 385 | O | THR | A | 386 | 35.149 | 62.326 | 1.911 | 1.00 | 44.06 |
| 386 | CE | THR | A | 386 | 33.664 | 63.226 | 0.580 | 1.00 | 45.20 |
| 387 | OG1 | THR | A | 386 | 32.744 | 63.045 | 1.662 | 1.00 | 51.89 |
| 388 | CG2 | THR | A | 386 | 34.616 | 64.368 | 0.936 | 1.00 | 47.20 |
| 389 | N | ARG | A | 387 | 36.749 | 62.176 | 0.326 | 1.00 | 44.39 |
| 390 | CA | ARG | A | 387 | 37.870 | 62.378 | 1.233 | 1.00 | 46.68 |
| 391 | C | ARG | A | 387 | 38.520 | 63.763 | 1.070 | 1.00 | 47.05 |
| 392 | O | ARG | A | 387 | 38.750 | 64.228 | 0.049 | 1.00 | 46.65 |
| 393 | CE | ARG | A | 387 | 38.906 | 61.284 | 0.979 | 1.00 | 48.13 |
| 394 | CG | ARG | A | 387 | 40.112 | 61.330 | 1.890 | 1.00 | 52.77 |
| 395 | CD | ARG | A | 387 | 41.247 | 60.494 | 1.320 | 1.00 | 56.60 |
| 396 | NE | ARG | A | 387 | 42.459 | 60.622 | 2.121 | 1.00 | 60.65 |
| 397 | CZ | ARG | A | 387 | 42.705 | 59.917 | 3.218 | 1.00 | 61.97 |
| 398 | NH1 | ARG | A | 387 | 41.821 | 59.022 | 3.636 | 1.00 | 64.45 |
| 399 | NH2 | ARG | A | 387 | 43.819 | 60.131 | 3.911 | 1.00 | 60.54 |
| 400 | N | LYS | A | 388 | 38.820 | 64.415 | 2.190 | 1.00 | 46.93 |
| 401 | CA | LYS | A | 388 | 39.457 | 65.730 | 2.167 | 1.00 | 47.51 |
| 402 | C | LYS | A | 388 | 40.630 | 65.777 | 3.143 | 1.00 | 48.33 |
| 403 | O | LYS | A | 388 | 40.493 | 65.401 | 4.306 | 1.00 | 46.08 |
| 404 | CB | LYS | A | 388 | 38.452 | 66.823 | 2.537 | 1.00 | 46.64 |
| 405 | CG | LYS | A | 388 | 37.274 | 66.909 | 1.603 | 1.00 | 52.75 |
| 406 | CD | LYS | A | 388 | 36.154 | 67.739 | 2.195 | 1.00 | 58.62 |
| 407 | CE | LYS | A | 388 | 34.863 | 67.538 | 1.418 | 1.00 | 62.49 |
| 408 | NZ | LYS | A | 388 | 33.719 | 68.204 | 2.101 | 1.00 | 67.33 |
| 409 | N | GLU | A | 389 | 41.780 | 66.236 | 2.651 | 1.00 | 50.07 |
| 410 | CA | GLU | A | 389 | 42.998 | 66.357 | 3.454 | 1.00 | 51.86 |
| 411 | C | GLU | A | 389 | 43.474 | 67.807 | 3.462 | 1.00 | 52.81 |
| 412 | O | GLU | A | 389 | 43.683 | 68.408 | 2.405 | 1.00 | 49.21 |
| 413 | CB | GLU | A | 389 | 44.106 | 65.474 | 2.877 | 1.00 | 54.67 |
| 414 | CG | GLU | A | 389 | 43.890 | 63.984 | 3.056 | 1.00 | 65.65 |
| 415 | CD | GLU | A | 389 | 44.992 | 63.159 | 2.412 | 1.00 | 72.66 |
| 416 | OE1 | GLU | A | 389 | 46.182 | 63.449 | 2.671 | 1.00 | 75.92 |
| 417 | OE2 | GLU | A | 389 | 44.669 | 62.220 | 1.650 | 1.00 | 75.78 |
| 418 | N | GLU | A | 390 | 43.652 | 68.371 | 4.651 | 1.00 | 56.06 |
| 419 | CA | GLU | A | 390 | 44.100 | 69.751 | 4.754 | 1.00 | 61.17 |
| 420 | C | GLU | A | 390 | 45.146 | 69.971 | 5.835 | 1.00 | 64.70 |
| 421 | O | GLU | A | 390 | 45.002 | 69.528 | 6.977 | 1.00 | 64.01 |
| 422 | CB | GLU | A | 390 | 42.908 | 70.673 | 4.993 | 1.00 | 64.35 |
| 423 | CG | GLU | A | 390 | 43.283 | 72.121 | 5.233 | 1.00 | 74.80 |
| 424 | CD | GLU | A | 390 | 42.091 | 73.052 | 5.121 | 1.00 | 85.97 |
| 425 | OE1 | GLU | A | 390 | 41.053 | 72.778 | 5.764 | 1.00 | 92.60 |
| 426 | OE2 | GLU | A | 390 | 42.197 | 74.059 | 4.388 | 1.00 | 92.18 |
| 427 | N | LYS | A | 391 | 46.204 | 70.679 | 5.461 | 1.00 | 68.18 |
| 428 | CA | LYS | A | 391 | 47.288 | 70.960 | 6.388 | 1.00 | 71.58 |
| 429 | C | LYS | A | 391 | 47.298 | 72.385 | 6.920 | 1.00 | 76.11 |
| 430 | O | LYS | A | 391 | 46.927 | 73.335 | 6.231 | 1.00 | 75.89 |
| 431 | CB | LYS | A | 391 | 48.627 | 70.654 | 5.728 | 1.00 | 67.28 |
| 432 | N | GLN | A | 392 | 47.724 | 72.508 | 8.168 | 1.00 | 81.85 |
| 433 | CA | GLN | A | 392 | 47.854 | 73.786 | 8.846 | 1.00 | 88.38 |
| 434 | C | GLN | A | 392 | 49.118 | 73.574 | 9.657 | 1.00 | 92.30 |
| 435 | O | GLN | A | 392 | 49.100 | 72.848 | 10.648 | 1.00 | 93.25 |
| 436 | CB | GLN | A | 392 | 46.662 | 74.045 | 9.777 | 1.00 | 85.87 |
| 437 | CG | GLN | A | 392 | 45.752 | 75.178 | 9.318 | 1.00 | 90.76 |
| 438 | CD | GLN | A | 392 | 44.727 | 75.573 | 10.369 | 1.00 | 93.39 |
| 439 | OE1 | GLN | A | 392 | 45.076 | 75.913 | 11.502 | 1.00 | 92.93 |
| 440 | NE2 | GLN | A | 392 | 43.453 | 75.538 | 9.993 | 1.00 | 93.97 |
| 441 | N | ARG | A | 393 | 50.221 | 74.174 | 9.220 | 1.00 | 96.24 |
| 442 | CA | ARG | A | 393 | 51.492 | 74.011 | 9.916 | 1.00 | 100.53 |
| 443 | C | ARG | A | 393 | 51.412 | 74.350 | 11.403 | 1.00 | 102.30 |
| 444 | O | ARG | A | 393 | 52.422 | 74.327 | 12.107 | 1.00 | 101.15 |
| 445 | CB | ARG | A | 393 | 52.579 | 74.853 | 9.245 | 1.00 | 106.99 |
| 446 | CG | ARG | A | 393 | 52.404 | 76.349 | 9.396 | 1.00 | 109.77 |
| 447 | CD | ARG | A | 393 | 53.493 | 77.079 | 8.636 | 1.00 | 115.73 |
| 448 | NE | ARG | A | 393 | 53.515 | 78.505 | 8.935 | 1.00 | 119.73 |
| 449 | CZ | ARG | A | 393 | 54.333 | 79.373 | 8.350 | 1.00 | 121.60 |
| 450 | NH1 | ARG | A | 393 | 55.194 | 78.955 | 7.430 | 1.00 | 121.59 |
| 451 | NH2 | ARG | A | 393 | 54.292 | 80.655 | 8.684 | 1.00 | 121.49 |
| 452 | N | ASN | A | 394 | 50.209 | 74.658 | 11.876 | 1.00 | 103.98 |
| 453 | CA | ASN | A | 394 | 50.005 | 74.970 | 13.281 | 1.00 | 106.60 |
| 454 | C | ASN | A | 394 | 50.275 | 73.682 | 14.051 | 1.00 | 106.89 |
| 455 | O | ASN | A | 394 | 51.084 | 73.650 | 14.978 | 1.00 | 108.55 |
| 456 | CB | ASN | A | 394 | 48.564 | 75.427 | 13.520 | 1.00 | 108.97 |
| 457 | CG | ASN | A | 394 | 48.326 | 75.891 | 14.947 | 1.00 | 112.83 |
| 458 | OD1 | ASN | A | 394 | 47.190 | 76.141 | 15.348 | 1.00 | 114.53 |
| 459 | ND2 | ASN | A | 394 | 49.402 | 76.016 | 15.719 | 1.00 | 114.40 |
| 460 | N | GLY | A | 395 | 49.593 | 72.616 | 13.648 | 1.00 | 105.53 |
| 461 | CA | GLY | A | 395 | 49.771 | 71.331 | 14.298 | 1.00 | 101.15 |
| 462 | C | GLY | A | 395 | 48.585 | 70.423 | 14.050 | 1.00 | 97.82 |
| 463 | O | GLY | A | 395 | 48.125 | 69.721 | 14.952 | 1.00 | 99.41 |
| 464 | N | THR | A | 396 | 48.084 | 70.423 | 12.822 | 1.00 | 94.50 |
| 465 | CA | THR | A | 396 | 46.937 | 69.589 | 12.522 | 1.00 | 89.02 |
| 466 | C | THR | A | 396 | 46.763 | 69.174 | 11.071 | 1.00 | 83.93 |
| 467 | O | THR | A | 396 | 46.477 | 70.000 | 10.204 | 1.00 | 83.27 |
| 468 | CB | THR | A | 396 | 45.625 | 70.278 | 12.970 | 1.00 | 92.03 |
| 469 | OG1 | THR | A | 396 | 45.648 | 70.484 | 14.388 | 1.00 | 94.17 |
| 470 | CG2 | THR | A | 396 | 44.414 | 69.421 | 12.596 | 1.00 | 95.48 |
| 471 | N | LEU | A | 397 | 46.942 | 67.882 | 10.821 | 1.00 | 79.69 |
| 472 | CA | LEU | A | 397 | 46.729 | 67.320 | 9.500 | 1.00 | 74.75 |
| 473 | C | LEU | A | 397 | 45.295 | 66.827 | 9.604 | 1.00 | 71.50 |
| 474 | O | LEU | A | 397 | 45.052 | 65.720 | 10.081 | 1.00 | 74.81 |
| 475 | CB | LEU | A | 397 | 47.640 | 66.120 | 9.240 | 1.00 | 75.72 |
| 476 | CG | LEU | A | 397 | 47.188 | 65.250 | 8.053 | 1.00 | 70.67 |
| 477 | CD1 | LEU | A | 397 | 47.668 | 65.858 | 6.738 | 1.00 | 69.05 |
| 478 | CD2 | LEU | A | 397 | 47.723 | 63.832 | 8.213 | 1.00 | 72.96 |
| 479 | N | THR | A | 398 | 44.348 | 67.659 | 9.195 | 1.00 | 67.61 |
| 480 | CA | THR | A | 398 | 42.945 | 67.286 | 9.252 | 1.00 | 61.30 |
| 481 | C | THR | A | 398 | 42.559 | 66.393 | 8.076 | 1.00 | 56.61 |
| 482 | O | THR | A | 398 | 42.960 | 66.626 | 6.934 | 1.00 | 54.95 |
| 483 | CB | THR | A | 398 | 42.044 | 68.530 | 9.248 | 1.00 | 61.75 |
| 484 | OG1 | THR | A | 398 | 42.225 | 69.255 | 10.471 | 1.00 | 65.40 |
| 485 | CG2 | THR | A | 398 | 40.584 | 68.129 | 9.108 | 1.00 | 63.39 |
| 486 | N | VAL | A | 399 | 41.783 | 65.359 | 8.371 | 1.00 | 51.84 |
| 487 | CA | VAL | A | 399 | 41.321 | 64.437 | 7.349 | 1.00 | 48.05 |
| 488 | C | VAL | A | 399 | 39.870 | 64.085 | 7.610 | 1.00 | 44.81 |
| 489 | O | VAL | A | 399 | 39.502 | 63.689 | 8.719 | 1.00 | 44.41 |
| 490 | CB | VAL | A | 399 | 42.132 | 63.134 | 7.349 | 1.00 | 49.35 |
| 491 | CG1 | VAL | A | 399 | 41.590 | 62.191 | 6.295 | 1.00 | 49.43 |
| 492 | CG2 | VAL | A | 399 | 43.594 | 63.432 | 7.090 | 1.00 | 53.10 |
| 493 | N | THR | A | 400 | 39.036 | 64.246 | 6.596 | 1.00 | 42.44 |
| 494 | CA | THR | A | 400 | 37.635 | 63.909 | 6.757 | 1.00 | 40.44 |
| 495 | C | THR | A | 400 | 37.151 | 63.068 | 5.602 | 1.00 | 38.07 |
| 496 | O | THR | A | 400 | 37.692 | 63.117 | 4.498 | 1.00 | 36.13 |
| 497 | CB | THR | A | 400 | 36.731 | 65.152 | 6.834 | 1.00 | 39.64 |
| 498 | OG1 | THR | A | 400 | 36.638 | 65.755 | 5.539 | 1.00 | 43.72 |
| 499 | CG2 | THR | A | 400 | 37.278 | 66.158 | 7.822 | 1.00 | 38.32 |
| 500 | N | SER | A | 401 | 36.140 | 62.265 | 5.884 | 1.00 | 36.90 |
| 501 | CA | SER | A | 401 | 35.531 | 61.432 | 4.876 | 1.00 | 35.46 |
| 502 | C | SER | A | 401 | 34.043 | 61.612 | 5.084 | 1.00 | 33.95 |
| 503 | O | SER | A | 401 | 33.538 | 61.447 | 6.192 | 1.00 | 34.80 |
| 504 | CB | SER | A | 401 | 35.906 | 59.962 | 5.060 | 1.00 | 36.91 |
| 505 | OG | SER | A | 401 | 35.299 | 59.183 | 4.040 | 1.00 | 40.84 |
| 506 | N | THR | A | 402 | 33.352 | 61.965 | 4.014 | 1.00 | 32.28 |
| 507 | CA | THR | A | 402 | 31.928 | 62.178 | 4.068 | 1.00 | 31.85 |
| 508 | C | THR | A | 402 | 31.245 | 61.092 | 3.264 | 1.00 | 31.76 |
| 509 | O | THR | A | 402 | 31.554 | 60.863 | 2.090 | 1.00 | 30.55 |
| 510 | CB | THR | A | 402 | 31.570 | 63.549 | 3.505 | 1.00 | 31.33 |
| 511 | OG1 | THR | A | 402 | 32.284 | 64.542 | 4.238 | 1.00 | 32.83 |
| 512 | CG2 | THR | A | 402 | 30.078 | 63.818 | 3.632 | 1.00 | 32.70 |
| 513 | N | LEU | A | 403 | 30.299 | 60.433 | 3.912 | 1.00 | 30.72 |
| 514 | CA | LEU | A | 403 | 29.582 | 59.343 | 3.300 | 1.00 | 30.33 |
| 515 | C | LEU | A | 403 | 28.115 | 59.673 | 3.097 | 1.00 | 29.66 |
| 516 | O | LEU | A | 403 | 27.415 | 59.992 | 4.052 | 1.00 | 28.06 |
| 517 | CB | LEU | A | 403 | 29.698 | 58.098 | 4.187 | 1.00 | 28.34 |
| 518 | CG | LEU | A | 403 | 28.968 | 56.831 | 3.719 | 1.00 | 32.10 |
| 519 | CG1 | LEU | A | 403 | 29.777 | 56.175 | 2.597 | 1.00 | 31.94 |
| 520 | CD2 | LEU | A | 403 | 28.810 | 55.844 | 4.876 | 1.00 | 30.42 |
| 521 | N | PRO | A | 404 | 27.638 | 59.617 | 1.841 | 1.00 | 31.48 |
| 522 | CA | PRO | A | 404 | 26.233 | 59.896 | 1.534 | 1.00 | 33.20 |
| 523 | C | PRO | A | 404 | 25.451 | 58.759 | 2.194 | 1.00 | 34.02 |
| 524 | O | PRO | A | 404 | 25.845 | 57.601 | 2.104 | 1.00 | 32.25 |
| 525 | CB | PRO | A | 404 | 26.183 | 59.812 | 0.010 | 1.00 | 33.28 |
| 526 | CG | PRO | A | 404 | 27.590 | 60.165 | 0.400 | 1.00 | 31.82 |
| 527 | CD | PRO | A | 404 | 28.413 | 59.414 | 0.603 | 1.00 | 31.26 |
| 528 | N | VAL | A | 405 | 24.362 | 59.097 | 2.865 | 1.00 | 34.94 |
| 529 | CA | VAL | A | 405 | 23.556 | 58.117 | 3.564 | 1.00 | 38.63 |
| 530 | C | VAL | A | 405 | 22.128 | 58.059 | 3.032 | 1.00 | 40.02 |
| 531 | O | VAL | A | 405 | 21.573 | 59.063 | 2.598 | 1.00 | 40.31 |
| 532 | CB | VAL | A | 405 | 23.548 | 58.457 | 5.082 | 1.00 | 42.09 |
| 533 | CG1 | VAL | A | 405 | 22.128 | 58.490 | 5.632 | 1.00 | 43.95 |
| 534 | CG2 | VAL | A | 405 | 24.405 | 57.456 | 5.827 | 1.00 | 41.76 |
| 535 | N | GLY | A | 406 | 21.536 | 56.872 | 3.061 | 1.00 | 42.35 |
| 536 | CA | GLY | A | 406 | 20.168 | 56.741 | 2.597 | 1.00 | 43.35 |
| 537 | C | GLY | A | 406 | 19.246 | 57.422 | 3.587 | 1.00 | 43.87 |
| 538 | O | GLY | A | 406 | 19.446 | 57.314 | 4.797 | 1.00 | 42.68 |
| 539 | N | THR | A | 407 | 18.248 | 58.134 | 3.077 | 1.00 | 44.40 |
| 540 | CA | THR | A | 407 | 17.294 | 58.840 | 3.923 | 1.00 | 47.37 |
| 541 | C | THR | A | 407 | 16.473 | 57.887 | 4.778 | 1.00 | 48.27 |
| 542 | O | THR | A | 407 | 16.392 | 58.051 | 5.993 | 1.00 | 48.77 |
| 543 | CE | THR | A | 407 | 16.312 | 59.678 | 3.078 | 1.00 | 48.44 |
| 544 | OG1 | THR | A | 407 | 17.001 | 60.791 | 2.504 | 1.00 | 50.00 |
| 545 | CG2 | THR | A | 407 | 15.164 | 60.187 | 3.938 | 1.00 | 53.23 |
| 546 | N | ARG | A | 408 | 15.854 | 56.903 | 4.130 | 1.00 | 48.10 |
| 547 | CA | ARG | A | 408 | 15.021 | 55.935 | 4.821 | 1.00 | 48.54 |
| 548 | C | ARG | A | 408 | 15.827 | 55.060 | 5.765 | 1.00 | 48.48 |
| 549 | O | ARG | A | 408 | 15.418 | 54.831 | 6.903 | 1.00 | 47.34 |
| 550 | CE | ARG | A | 408 | 14.275 | 55.070 | 3.810 | 1.00 | 50.16 |
| 551 | N | ASP | A | 409 | 16.966 | 54.567 | 5.294 | 1.00 | 48.72 |
| 552 | CA | ASP | A | 409 | 17.813 | 53.714 | 6.121 | 1.00 | 50.88 |
| 553 | C | ASP | A | 409 | 18.179 | 54.413 | 7.426 | 1.00 | 49.92 |
| 554 | O | ASP | A | 409 | 18.132 | 53.811 | 8.502 | 1.00 | 49.76 |
| 555 | CE | ASP | A | 409 | 19.088 | 53.340 | 5.362 | 1.00 | 56.18 |
| 556 | CG | ASP | A | 409 | 18.799 | 52.587 | 4.074 | 1.00 | 64.80 |
| 557 | OD1 | ASP | A | 409 | 18.118 | 51.538 | 4.144 | 1.00 | 71.41 |
| 558 | OD2 | ASP | A | 409 | 19.247 | 53.039 | 2.995 | 1.00 | 68.29 |
| 559 | N | TRP | A | 410 | 18.533 | 55.692 | 7.327 | 1.00 | 48.36 |
| 560 | CA | TRP | A | 410 | 18.914 | 56.458 | 8.503 | 1.00 | 46.32 |
| 561 | C | TRP | A | 410 | 17.736 | 56.647 | 9.447 | 1.00 | 46.21 |
| 562 | O | TRP | A | 410 | 17.869 | 56.489 | 10.660 | 1.00 | 44.64 |
| 563 | CE | TRP | A | 410 | 19.480 | 57.826 | 8.108 | 1.00 | 42.82 |
| 564 | CG | TRP | A | 410 | 19.890 | 58.599 | 9.304 | 1.00 | 39.31 |
| 565 | CD1 | TRP | A | 410 | 19.133 | 59.496 | 9.997 | 1.00 | 37.29 |
| 566 | CD2 | TRP | A | 410 | 21.093 | 58.419 | 10.057 | 1.00 | 35.49 |
| 567 | NE1 | TRP | A | 410 | 19.781 | 59.874 | 11.143 | 1.00 | 39.24 |
| 568 | CE2 | TRP | A | 410 | 20.987 | 59.228 | 11.205 | 1.00 | 32.76 |
| 569 | CE3 | TRP | A | 410 | 22.249 | 57.644 | 9.872 | 1.00 | 32.07 |
| 570 | CZ2 | TRP | A | 410 | 21.994 | 59.291 | 12.176 | 1.00 | 29.59 |
| 571 | CZ3 | TRP | A | 410 | 23.248 | 57.704 | 10.831 | 1.00 | 33.68 |
| 572 | CH2 | TRP | A | 410 | 23.111 | 58.527 | 11.975 | 1.00 | 30.56 |
| 573 | N | ILE | A | 411 | 16.584 | 56.989 | 8.888 | 1.00 | 48.11 |
| 574 | CA | ILE | A | 411 | 15.391 | 57.192 | 9.699 | 1.00 | 50.90 |
| 575 | C | ILE | A | 411 | 14.951 | 55.888 | 10.358 | 1.00 | 52.46 |
| 576 | O | ILE | A | 411 | 14.371 | 55.902 | 11.442 | 1.00 | 53.34 |
| 577 | CB | ILE | A | 411 | 14.234 | 57.755 | 8.854 | 1.00 | 51.58 |
| 578 | CG1 | ILE | A | 411 | 14.613 | 59.144 | 8.330 | 1.00 | 52.81 |
| 579 | CG2 | ILE | A | 411 | 12.966 | 57.828 | 9.685 | 1.00 | 51.95 |
| 580 | CD1 | ILE | A | 411 | 13.566 | 59.777 | 7.431 | 1.00 | 59.38 |
| 581 | N | GLU | A | 412 | 15.252 | 54.763 | 9.715 | 1.00 | 53.21 |
| 582 | CA | GLU | A | 412 | 14.872 | 53.463 | 10.248 | 1.00 | 53.12 |
| 583 | C | GLU | A | 412 | 15.853 | 52.919 | 11.269 | 1.00 | 51.24 |
| 584 | O | GLU | A | 412 | 15.610 | 51.864 | 11.848 | 1.00 | 51.40 |
| 585 | CB | GLU | A | 412 | 14.691 | 52.448 | 9.119 | 1.00 | 59.71 |
| 586 | CG | GLU | A | 412 | 13.362 | 52.578 | 8.383 | 1.00 | 70.52 |
| 587 | CD | GLU | A | 412 | 13.201 | 51.540 | 7.290 | 1.00 | 78.58 |
| 588 | OE1 | GLU | A | 412 | 13.402 | 50.338 | 7.573 | 1.00 | 83.44 |
| 589 | OE2 | GLU | A | 412 | 12.868 | 51.924 | 6.150 | 1.00 | 82.10 |
| 590 | N | GLY | A | 413 | 16.966 | 53.618 | 11.477 | 1.00 | 48.47 |
| 591 | CA | GLY | A | 413 | 17.923 | 53.173 | 12.474 | 1.00 | 44.62 |
| 592 | C | GLY | A | 413 | 19.213 | 52.484 | 12.068 | 1.00 | 44.15 |
| 593 | O | GLY | A | 413 | 19.882 | 51.913 | 12.927 | 1.00 | 43.50 |
| 594 | N | GLU | A | 414 | 19.588 | 52.515 | 10.793 | 1.00 | 42.78 |
| 595 | CA | GLU | A | 414 | 20.843 | 51.878 | 10.406 | 1.00 | 40.78 |
| 596 | C | GLU | A | 414 | 21.948 | 52.495 | 11.249 | 1.00 | 39.97 |
| 597 | O | GLU | A | 414 | 21.842 | 53.645 | 11.690 | 1.00 | 37.43 |
| 598 | CB | GLU | A | 414 | 21.155 | 52.111 | 8.918 | 1.00 | 41.96 |
| 599 | CG | GLU | A | 414 | 22.519 | 51.566 | 8.441 | 1.00 | 44.09 |
| 600 | CD | GLU | A | 414 | 22.582 | 50.036 | 8.360 | 1.00 | 49.01 |
| 601 | OE1 | GLU | A | 414 | 23.051 | 49.387 | 9.325 | 1.00 | 46.84 |
| 602 | OE2 | GLU | A | 414 | 22.152 | 49.479 | 7.327 | 1.00 | 49.89 |
| 603 | N | THR | A | 415 | 23.001 | 51.721 | 11.485 | 1.00 | 39.56 |
| 604 | CA | THR | A | 415 | 24.136 | 52.197 | 12.253 | 1.00 | 40.76 |
| 605 | C | THR | A | 415 | 25.369 | 52.113 | 11.362 | 1.00 | 40.07 |
| 606 | O | THR | A | 415 | 25.640 | 51.067 | 10.777 | 1.00 | 42.10 |
| 607 | CB | THR | A | 415 | 24.327 | 51.362 | 13.544 | 1.00 | 44.69 |
| 608 | OG1 | THR | A | 415 | 25.707 | 51.378 | 13.929 | 1.00 | 51.98 |
| 609 | CG2 | THR | A | 415 | 23.856 | 49.948 | 13.342 | 1.00 | 50.00 |
| 610 | N | TYR | A | 416 | 26.100 | 53.220 | 11.240 | 1.00 | 37.57 |
| 611 | CA | TYR | A | 416 | 27.282 | 53.273 | 10.386 | 1.00 | 34.50 |
| 612 | C | TYR | A | 416 | 28.549 | 53.335 | 11.201 | 1.00 | 36.77 |
| 613 | O | TYR | A | 416 | 28.562 | 53.894 | 12.300 | 1.00 | 35.94 |
| 614 | CB | TYR | A | 416 | 27.221 | 54.484 | 9.466 | 1.00 | 34.15 |
| 615 | CG | TYR | A | 416 | 26.003 | 54.515 | 8.586 | 1.00 | 30.65 |
| 616 | CD1 | TYR | A | 416 | 24.744 | 54.823 | 9.108 | 1.00 | 32.53 |
| 617 | CD2 | TYR | A | 416 | 26.101 | 54.211 | 7.235 | 1.00 | 31.72 |
| 618 | CE1 | TYR | A | 416 | 23.616 | 54.828 | 8.297 | 1.00 | 34.27 |
| 619 | CE2 | TYR | A | 416 | 24.981 | 54.210 | 6.420 | 1.00 | 32.11 |
| 620 | CZ | TYR | A | 416 | 23.744 | 54.520 | 6.956 | 1.00 | 33.86 |
| 621 | OH | TYR | A | 416 | 22.636 | 54.518 | 6.139 | 1.00 | 41.22 |
| 622 | N | GLN | A | 417 | 29.624 | 52.771 | 10.659 | 1.00 | 37.58 |
| 623 | CA | GLN | A | 417 | 30.878 | 52.752 | 11.386 | 1.00 | 39.43 |
| 624 | C | GLN | A | 417 | 32.062 | 53.259 | 10.582 | 1.00 | 39.40 |
| 625 | O | GLN | A | 417 | 32.227 | 52.936 | 9.406 | 1.00 | 39.63 |
| 626 | CB | GLN | A | 417 | 31.179 | 51.331 | 11.889 | 1.00 | 41.37 |
| 627 | CG | GLN | A | 417 | 32.386 | 51.250 | 12.822 | 1.00 | 51.81 |
| 628 | CD | GLN | A | 417 | 32.744 | 49.827 | 13.214 | 1.00 | 61.69 |
| 629 | OE1 | GLN | A | 417 | 33.229 | 49.045 | 12.390 | 1.00 | 67.15 |
| 630 | NE2 | GLN | A | 417 | 32.504 | 49.481 | 14.479 | 1.00 | 63.64 |
| 631 | N | CYS | A | 418 | 32.888 | 54.057 | 11.236 | 1.00 | 39.29 |
| 632 | CA | CYS | A | 418 | 34.083 | 54.581 | 10.611 | 1.00 | 41.80 |
| 633 | C | CYS | A | 418 | 35.250 | 53.880 | 11.282 | 1.00 | 42.50 |
| 634 | O | CYS | A | 418 | 35.409 | 53.954 | 12.500 | 1.00 | 43.33 |
| 635 | CB | CYS | A | 418 | 34.208 | 56.099 | 10.825 | 1.00 | 41.95 |
| 636 | SG | CYS | A | 418 | 35.696 | 56.805 | 10.043 | 1.00 | 54.15 |
| 637 | N | ARG | A | 419 | 36.049 | 53.182 | 10.491 | 1.00 | 43.98 |
| 638 | CA | ARG | A | 419 | 37.225 | 52.497 | 11.007 | 1.00 | 47.51 |
| 639 | C | ARG | A | 419 | 38.428 | 53.323 | 10.558 | 1.00 | 48.11 |
| 640 | O | ARG | A | 419 | 38.741 | 53.381 | 9.370 | 1.00 | 47.85 |
| 641 | CB | ARG | A | 419 | 37.289 | 51.068 | 10.452 | 1.00 | 49.08 |
| 642 | CG | ARG | A | 419 | 38.642 | 50.397 | 10.586 | 1.00 | 54.11 |
| 643 | CD | ARG | A | 419 | 38.554 | 48.892 | 10.304 | 1.00 | 64.71 |
| 644 | NE | ARG | A | 419 | 39.868 | 48.275 | 10.137 | 1.00 | 64.09 |
| 645 | CZ | ARG | A | 419 | 40.493 | 48.163 | 8.968 | 1.00 | 70.31 |
| 646 | NH1 | ARG | A | 419 | 39.919 | 48.619 | 7.861 | 1.00 | 68.32 |
| 647 | NH2 | ARG | A | 419 | 41.699 | 47.608 | 8.905 | 1.00 | 71.53 |
| 648 | N | VAL | A | 420 | 39.076 | 53.990 | 11.508 | 1.00 | 51.04 |
| 649 | CA | VAL | A | 420 | 40.231 | 54.824 | 11.200 | 1.00 | 56.22 |
| 650 | C | VAL | A | 420 | 41.532 | 54.039 | 11.354 | 1.00 | 61.25 |
| 651 | O | VAL | A | 420 | 41.780 | 53.422 | 12.393 | 1.00 | 60.11 |
| 652 | CB | VAL | A | 420 | 40.295 | 56.064 | 12.115 | 1.00 | 54.44 |
| 653 | CG1 | VAL | A | 420 | 41.494 | 56.922 | 11.729 | 1.00 | 52.69 |
| 654 | CG2 | VAL | A | 420 | 39.003 | 56.868 | 12.005 | 1.00 | 55.45 |
| 655 | N | THR | A | 421 | 42.359 | 54.071 | 10.314 | 1.00 | 66.23 |
| 656 | CA | THR | A | 421 | 43.631 | 53.356 | 10.326 | 1.00 | 73.18 |
| 657 | C | THR | A | 421 | 44.834 | 54.266 | 10.130 | 1.00 | 76.87 |
| 658 | O | THR | A | 421 | 45.157 | 54.632 | 9.000 | 1.00 | 77.93 |
| 659 | CB | THR | A | 421 | 43.671 | 52.283 | 9.225 | 1.00 | 73.75 |
| 660 | OG1 | THR | A | 421 | 42.628 | 51.328 | 9.451 | 1.00 | 77.71 |
| 661 | CG2 | THR | A | 421 | 45.009 | 51.567 | 9.227 | 1.00 | 75.35 |
| 662 | N | HIS | A | 422 | 45.499 | 54.624 | 11.226 | 1.00 | 81.02 |
| 663 | CA | HIS | A | 422 | 46.678 | 55.483 | 11.151 | 1.00 | 86.07 |
| 664 | C | HIS | A | 422 | 47.914 | 54.596 | 11.278 | 1.00 | 89.67 |
| 665 | O | HIS | A | 422 | 47.947 | 53.680 | 12.098 | 1.00 | 90.00 |
| 666 | CB | HIS | A | 422 | 46.660 | 56.529 | 12.269 | 1.00 | 87.43 |
| 667 | CG | HIS | A | 422 | 47.548 | 57.708 | 12.008 | 1.00 | 90.73 |
| 668 | ND1 | HIS | A | 422 | 47.397 | 58.524 | 10.909 | 1.00 | 93.83 |
| 669 | CD2 | HIS | A | 422 | 48.600 | 58.204 | 12.704 | 1.00 | 91.89 |
| 670 | CE1 | HIS | A | 422 | 48.318 | 59.473 | 10.936 | 1.00 | 94.70 |
| 671 | NE2 | HIS | A | 422 | 49.060 | 59.300 | 12.015 | 1.00 | 93.25 |
| 672 | N | PRO | A | 423 | 48.954 | 54.871 | 10.474 | 1.00 | 92.64 |
| 673 | CA | PRO | A | 423 | 50.209 | 54.111 | 10.460 | 1.00 | 95.19 |
| 674 | C | PRO | A | 423 | 50.838 | 53.628 | 11.775 | 1.00 | 97.06 |
| 675 | O | PRO | A | 423 | 51.064 | 52.431 | 11.945 | 1.00 | 97.57 |
| 676 | CB | PRO | A | 423 | 51.155 | 55.024 | 9.667 | 1.00 | 95.56 |
| 677 | CG | PRO | A | 423 | 50.566 | 56.391 | 9.853 | 1.00 | 93.94 |
| 678 | CD | PRO | A | 423 | 49.105 | 56.112 | 9.696 | 1.00 | 93.12 |
| 679 | N | HIS | A | 424 | 51.105 | 54.542 | 12.701 | 1.00 | 98.71 |
| 680 | CA | HIS | A | 424 | 51.769 | 54.190 | 13.957 | 1.00 | 100.16 |
| 681 | C | HIS | A | 424 | 51.021 | 53.496 | 15.095 | 1.00 | 99.67 |
| 682 | O | HIS | A | 424 | 51.609 | 52.690 | 15.816 | 1.00 | 99.61 |
| 683 | CB | HIS | A | 424 | 52.451 | 55.435 | 14.522 | 1.00 | 102.42 |
| 684 | CG | HIS | A | 424 | 53.475 | 56.022 | 13.605 | 1.00 | 105.47 |
| 685 | ND1 | HIS | A | 424 | 53.372 | 57.298 | 13.092 | 1.00 | 107.10 |
| 686 | CD2 | HIS | A | 424 | 54.639 | 55.521 | 13.133 | 1.00 | 97.46 |
| 687 | CE1 | HIS | A | 424 | 54.430 | 57.556 | 12.345 | 1.00 | 106.81 |
| 688 | NE2 | HIS | A | 424 | 55.215 | 56.494 | 12.353 | 1.00 | 106.61 |
| 689 | N | LEU | A | 425 | 49.742 | 53.798 | 15.267 | 1.00 | 99.19 |
| 690 | CA | LEU | A | 425 | 48.972 | 53.216 | 16.363 | 1.00 | 99.01 |
| 691 | C | LEU | A | 425 | 48.692 | 51.714 | 16.284 | 1.00 | 98.36 |
| 692 | O | LEU | A | 425 | 48.479 | 51.160 | 15.204 | 1.00 | 98.39 |
| 693 | CB | LEU | A | 425 | 47.671 | 53.994 | 16.507 | 1.00 | 100.66 |
| 694 | CG | LEU | A | 425 | 47.969 | 55.495 | 16.467 | 1.00 | 101.95 |
| 695 | CD1 | LEU | A | 425 | 46.683 | 56.285 | 16.569 | 1.00 | 102.59 |
| 696 | CD2 | LEU | A | 425 | 48.923 | 55.856 | 17.598 | 1.00 | 103.85 |
| 697 | N | PRO | A | 426 | 48.685 | 51.038 | 17.448 | 1.00 | 97.57 |
| 698 | CA | PRO | A | 426 | 48.435 | 49.596 | 17.540 | 1.00 | 96.82 |
| 699 | C | PRO | A | 426 | 47.075 | 49.186 | 16.992 | 1.00 | 95.53 |
| 700 | O | PRO | A | 426 | 46.990 | 48.560 | 15.937 | 1.00 | 95.77 |
| 701 | CB | PRO | A | 426 | 48.570 | 49.318 | 19.037 | 1.00 | 97.13 |
| 702 | CG | PRO | A | 426 | 48.106 | 50.605 | 19.660 | 1.00 | 97.88 |
| 703 | CG | PRO | A | 426 | 48.802 | 51.631 | 18.793 | 1.00 | 97.70 |
| 704 | N | ARG | A | 427 | 46.015 | 49.536 | 17.713 | 1.00 | 94.16 |
| 705 | CA | ARG | A | 427 | 44.669 | 49.198 | 17.274 | 1.00 | 92.52 |
| 706 | C | ARG | A | 427 | 44.091 | 50.315 | 16.408 | 1.00 | 89.85 |
| 707 | O | ARG | A | 427 | 44.487 | 51.477 | 16.517 | 1.00 | 89.26 |
| 708 | CB | ARG | A | 427 | 43.749 | 48.948 | 18.478 | 1.00 | 97.34 |
| 709 | CG | ARG | A | 427 | 42.381 | 48.398 | 18.077 | 1.00 | 99.32 |
| 710 | CD | ARG | A | 427 | 41.428 | 48.193 | 19.253 | 1.00 | 105.99 |
| 711 | NE | ARG | A | 427 | 40.197 | 47.532 | 18.815 | 1.00 | 111.10 |
| 712 | CZ | ARG | A | 427 | 39.140 | 47.298 | 19.588 | 1.00 | 111.66 |
| 713 | NH1 | ARG | A | 427 | 39.143 | 47.673 | 20.861 | 1.00 | 111.70 |
| 714 | NH2 | ARG | A | 427 | 38.078 | 46.679 | 19.087 | 1.00 | 110.09 |
| 715 | N | ALA | A | 428 | 43.158 | 49.949 | 15.538 | 1.00 | 86.56 |
| 716 | CA | ALA | A | 428 | 42.513 | 50.910 | 14.661 | 1.00 | 82.70 |
| 717 | C | ALA | A | 428 | 41.389 | 51.602 | 15.423 | 1.00 | 80.03 |
| 718 | O | ALA | A | 428 | 40.711 | 50.981 | 16.239 | 1.00 | 80.29 |
| 719 | CB | ALA | A | 428 | 41.953 | 50.200 | 13.437 | 1.00 | 83.85 |
| 720 | N | LEU | A | 429 | 41.203 | 52.892 | 15.164 | 1.00 | 76.76 |
| 721 | CA | LEU | A | 429 | 40.144 | 53.651 | 15.815 | 1.00 | 72.72 |
| 722 | C | LEU | A | 429 | 38.820 | 53.289 | 15.164 | 1.00 | 69.67 |
| 723 | O | LEU | A | 429 | 38.742 | 53.128 | 13.947 | 1.00 | 68.64 |
| 724 | CB | LEU | A | 429 | 40.392 | 55.156 | 15.671 | 1.00 | 73.60 |
| 725 | CG | LEU | A | 429 | 41.169 | 55.847 | 16.793 | 1.00 | 73.35 |
| 726 | CD1 | LEU | A | 429 | 41.498 | 57.280 | 16.404 | 1.00 | 72.09 |
| 727 | CD2 | LEU | A | 429 | 40.333 | 55.813 | 18.065 | 1.00 | 76.40 |
| 728 | N | MET | A | 430 | 37.779 | 53.153 | 15.976 | 1.00 | 66.83 |
| 729 | CA | MET | A | 430 | 36.468 | 52.814 | 15.449 | 1.00 | 64.81 |
| 730 | C | MET | A | 430 | 35.384 | 53.678 | 16.074 | 1.00 | 61.41 |
| 731 | O | MET | A | 430 | 35.274 | 53.776 | 17.299 | 1.00 | 60.84 |
| 732 | CB | MET | A | 430 | 36.173 | 51.335 | 15.691 | 1.00 | 68.22 |
| 733 | CG | MET | A | 430 | 37.247 | 50.416 | 15.133 | 1.00 | 74.39 |
| 734 | SD | MET | A | 430 | 36.886 | 48.679 | 15.387 | 1.00 | 83.18 |
| 735 | CE | MET | A | 430 | 36.836 | 48.607 | 17.194 | 1.00 | 84.12 |
| 736 | N | ARG | A | 431 | 34.590 | 54.312 | 15.218 | 1.00 | 57.17 |
| 737 | CA | ARG | A | 431 | 33.506 | 55.172 | 15.674 | 1.00 | 54.04 |
| 738 | C | ARG | A | 431 | 32.234 | 54.752 | 14.963 | 1.00 | 50.58 |
| 739 | O | ARG | A | 431 | 32.270 | 54.367 | 13.796 | 1.00 | 50.46 |
| 740 | CB | ARG | A | 431 | 33.825 | 56.641 | 15.364 | 1.00 | 55.16 |
| 741 | CG | ARG | A | 431 | 35.036 | 57.187 | 16.109 | 1.00 | 59.24 |
| 742 | CD | ARG | A | 431 | 34.802 | 57.172 | 17.611 | 1.00 | 63.93 |
| 743 | NE | ARG | A | 431 | 35.983 | 57.586 | 18.363 | 1.00 | 70.30 |
| 744 | CZ | ARG | A | 431 | 36.070 | 57.557 | 19.689 | 1.00 | 76.24 |
| 745 | NH1 | ARG | A | 431 | 35.041 | 57.131 | 20.417 | 1.00 | 77.83 |
| 746 | NH2 | ARG | A | 431 | 37.185 | 57.952 | 20.289 | 1.00 | 77.12 |
| 747 | N | SER | A | 432 | 31.112 | 54.812 | 15.669 | 1.00 | 46.92 |
| 748 | CA | SER | A | 432 | 29.835 | 54.437 | 15.084 | 1.00 | 45.15 |
| 749 | C | SER | A | 432 | 28.784 | 55.482 | 15.398 | 1.00 | 42.49 |
| 750 | O | SER | A | 432 | 28.901 | 56.218 | 16.379 | 1.00 | 42.76 |
| 751 | CB | SER | A | 432 | 29.394 | 53.068 | 15.610 | 1.00 | 44.31 |
| 752 | OC | SER | A | 432 | 29.316 | 53.084 | 17.018 | 1.00 | 49.96 |
| 753 | N | THR | A | 433 | 27.749 | 55.540 | 14.570 | 1.00 | 40.28 |
| 754 | CA | THR | A | 433 | 26.691 | 56.522 | 14.763 | 1.00 | 39.71 |
| 755 | C | THR | A | 433 | 25.356 | 55.996 | 14.257 | 1.00 | 39.49 |
| 756 | O | THR | A | 433 | 25.315 | 55.186 | 13.334 | 1.00 | 40.22 |
| 757 | CB | THR | A | 433 | 27.023 | 57.839 | 14.022 | 1.00 | 38.07 |
| 758 | OG1 | THR | A | 433 | 26.006 | 58.807 | 14.295 | 1.00 | 39.56 |
| 759 | CG2 | THR | A | 433 | 27.088 | 57.609 | 12.518 | 1.00 | 36.99 |
| 760 | N | THR | A | 434 | 24.272 | 56.461 | 14.874 | 1.00 | 39.18 |
| 761 | CA | THR | A | 434 | 22.913 | 56.068 | 14.512 | 1.00 | 40.70 |
| 762 | C | THR | A | 434 | 21.965 | 57.186 | 14.934 | 1.00 | 40.23 |
| 763 | O | THR | A | 434 | 22.364 | 58.091 | 15.659 | 1.00 | 41.18 |
| 764 | CB | THR | A | 434 | 22.484 | 54.784 | 15.256 | 1.00 | 43.01 |
| 765 | OG1 | THR | A | 434 | 22.362 | 55.052 | 16.661 | 1.00 | 47.85 |
| 766 | CG2 | THR | A | 434 | 23.524 | 53.726 | 15.105 | 1.00 | 48.67 |
| 767 | N | LYS | A | 435 | 20.714 | 57.125 | 14.496 | 1.00 | 40.97 |
| 768 | CA | LYS | A | 435 | 19.767 | 58.150 | 14.889 | 1.00 | 45.73 |
| 769 | C | LYS | A | 435 | 19.601 | 58.021 | 16.398 | 1.00 | 48.93 |
| 770 | O | LYS | A | 435 | 19.710 | 56.930 | 16.942 | 1.00 | 48.49 |
| 771 | CE | LYS | A | 435 | 18.434 | 57.937 | 14.194 | 1.00 | 45.94 |
| 772 | N | THR | A | 436 | 19.365 | 59.133 | 17.082 | 1.00 | 52.01 |
| 773 | CA | THR | A | 436 | 19.171 | 59.080 | 18.526 | 1.00 | 54.88 |
| 774 | C | THR | A | 436 | 17.722 | 58.684 | 18.774 | 1.00 | 55.70 |
| 775 | O | THR | A | 436 | 16.823 | 59.146 | 18.077 | 1.00 | 55.21 |
| 776 | CB | THR | A | 436 | 19.438 | 60.454 | 19.191 | 1.00 | 56.24 |
| 777 | OG1 | THR | A | 436 | 20.821 | 60.802 | 19.038 | 1.00 | 60.56 |
| 778 | CG2 | THR | A | 436 | 19.090 | 60.412 | 20.677 | 1.00 | 57.04 |
| 779 | N | SER | A | 437 | 17.501 | 57.810 | 19.748 | 1.00 | 57.93 |
| 780 | CA | SER | A | 437 | 16.149 | 57.380 | 20.080 | 1.00 | 60.42 |
| 781 | C | SER | A | 437 | 15.683 | 58.239 | 21.249 | 1.00 | 60.49 |
| 782 | O | SER | A | 437 | 16.357 | 59.203 | 21.623 | 1.00 | 60.21 |
| 783 | CB | SER | A | 437 | 16.133 | 55.894 | 20.470 | 1.00 | 61.57 |
| 784 | OG | SER | A | 437 | 16.957 | 55.651 | 21.600 | 1.00 | 65.51 |
| 785 | N | GLY | A | 438 | 14.533 | 57.895 | 21.821 | 1.00 | 60.09 |
| 786 | CA | GLY | A | 438 | 14.022 | 58.666 | 22.939 | 1.00 | 58.42 |
| 787 | C | GLY | A | 438 | 12.937 | 59.630 | 22.511 | 1.00 | 57.80 |
| 788 | O | GLY | A | 438 | 12.648 | 59.747 | 21.322 | 1.00 | 56.51 |
| 789 | N | PRO | A | 439 | 12.309 | 60.333 | 23.467 | 1.00 | 57.81 |
| 790 | CA | PRO | A | 439 | 11.242 | 61.295 | 23.185 | 1.00 | 56.19 |
| 791 | C | PRO | A | 439 | 11.692 | 62.422 | 22.263 | 1.00 | 53.91 |
| 792 | O | PRO | A | 439 | 12.886 | 62.709 | 22.147 | 1.00 | 53.91 |
| 793 | CE | PRO | A | 439 | 10.865 | 61.806 | 24.576 | 1.00 | 57.79 |
| 794 | CG | PRO | A | 439 | 11.147 | 60.633 | 25.448 | 1.00 | 58.60 |
| 795 | CD | PRO | A | 439 | 12.485 | 60.173 | 24.921 | 1.00 | 60.65 |
| 796 | N | ARG | A | 440 | 10.722 | 63.055 | 21.612 | 1.00 | 51.09 |
| 797 | CA | ARG | A | 440 | 10.990 | 64.163 | 20.702 | 1.00 | 48.90 |
| 798 | C | ARG | A | 440 | 10.220 | 65.365 | 21.231 | 1.00 | 45.83 |
| 799 | O | ARG | A | 440 | 9.188 | 65.202 | 21.881 | 1.00 | 45.88 |
| 800 | CE | ARG | A | 440 | 10.501 | 63.836 | 19.289 | 1.00 | 49.95 |
| 801 | CG | ARG | A | 440 | 10.919 | 62.473 | 18.771 | 1.00 | 59.03 |
| 802 | CD | ARG | A | 440 | 12.406 | 62.373 | 18.448 | 1.00 | 65.31 |
| 803 | NE | ARG | A | 440 | 12.814 | 60.973 | 18.342 | 1.00 | 72.31 |
| 804 | CZ | ARG | A | 440 | 13.986 | 60.553 | 17.878 | 1.00 | 74.99 |
| 804 | CZ | ARG | A | 440 | 13.986 | 60.553 | 17.878 | 1.00 | 74.99 |
| 805 | NH1 | ARG | A | 440 | 14.894 | 61.419 | 17.460 | 1.00 | 77.22 |
| 806 | NH2 | ARG | A | 440 | 14.252 | 59.255 | 17.837 | 1.00 | 78.59 |
| 807 | N | ALA | A | 441 | 10.727 | 66.564 | 20.957 | 1.00 | 42.09 |
| 808 | CA | ALA | A | 441 | 10.079 | 67.800 | 21.387 | 1.00 | 39.26 |
| 809 | C | ALA | A | 441 | 10.476 | 68.894 | 20.413 | 1.00 | 38.22 |
| 810 | O | ALA | A | 441 | 11.649 | 69.034 | 20.076 | 1.00 | 38.95 |
| 811 | CE | ALA | A | 441 | 10.511 | 68.170 | 22.798 | 1.00 | 36.56 |
| 812 | N | ALA | A | 442 | 9.496 | 69.666 | 19.966 | 1.00 | 37.22 |
| 813 | CA | ALA | A | 442 | 9.739 | 70.729 | 19.011 | 1.00 | 37.60 |
| 814 | C | ALA | A | 442 | 10.550 | 71.879 | 19.590 | 1.00 | 38.65 |
| 815 | O | ALA | A | 442 | 10.553 | 72.103 | 20.798 | 1.00 | 40.22 |
| 816 | CB | ALA | A | 442 | 8.415 | 71.248 | 18.481 | 1.00 | 37.17 |
| 817 | N | PRO | A | 443 | 11.260 | 72.619 | 18.722 | 1.00 | 38.04 |
| 818 | CA | PRO | A | 443 | 12.089 | 73.765 | 19.104 | 1.00 | 36.79 |
| 819 | C | PRO | A | 443 | 11.224 | 75.019 | 19.243 | 1.00 | 38.14 |
| 820 | O | PRO | A | 443 | 10.236 | 75.174 | 18.521 | 1.00 | 37.39 |
| 821 | CB | PRO | A | 443 | 13.050 | 73.923 | 17.921 | 1.00 | 36.30 |
| 822 | CG | PRO | A | 443 | 12.946 | 72.632 | 17.159 | 1.00 | 36.35 |
| 823 | CD | PRO | A | 443 | 11.512 | 72.249 | 17.320 | 1.00 | 37.85 |
| 824 | N | GLU | A | 444 | 11.594 | 75.894 | 20.173 | 1.00 | 36.28 |
| 825 | CA | GLU | A | 444 | 10.901 | 77.163 | 20.371 | 1.00 | 35.57 |
| 826 | C | GLU | A | 444 | 11.941 | 78.171 | 19.888 | 1.00 | 33.87 |
| 827 | O | GLU | A | 444 | 13.110 | 78.105 | 20.291 | 1.00 | 33.43 |
| 828 | CB | GLU | A | 444 | 10.580 | 77.383 | 21.851 | 1.00 | 38.68 |
| 829 | N | VAL | A | 445 | 11.520 | 79.097 | 19.033 | 1.00 | 30.18 |
| 830 | CA | VAL | A | 445 | 12.432 | 80.070 | 18.450 | 1.00 | 28.24 |
| 831 | C | VAL | A | 445 | 12.136 | 81.523 | 18.811 | 1.00 | 28.44 |
| 832 | O | VAL | A | 445 | 11.000 | 81.975 | 18.721 | 1.00 | 26.78 |
| 833 | CB | VAL | A | 445 | 12.417 | 79.937 | 16.905 | 1.00 | 28.10 |
| 834 | CG1 | VAL | A | 445 | 13.370 | 80.933 | 16.274 | 1.00 | 25.56 |
| 835 | CG2 | VAL | A | 445 | 12.771 | 78.521 | 16.511 | 1.00 | 26.30 |
| 836 | N | TYR | A | 446 | 13.175 | 82.251 | 19.203 | 1.00 | 27.87 |
| 837 | CA | TYR | A | 446 | 13.019 | 83.651 | 19.551 | 1.00 | 27.91 |
| 838 | C | TYR | A | 446 | 14.193 | 84.460 | 19.024 | 1.00 | 27.06 |
| 839 | O | TYR | A | 446 | 15.326 | 84.307 | 19.480 | 1.00 | 27.96 |
| 840 | CB | TYR | A | 446 | 12.898 | 83.836 | 21.074 | 1.00 | 26.45 |
| 841 | CG | TYR | A | 446 | 12.749 | 85.289 | 21.447 | 1.00 | 26.59 |
| 842 | CD1 | TYR | A | 446 | 11.714 | 86.057 | 20.905 | 1.00 | 28.48 |
| 843 | CD2 | TYR | A | 446 | 13.659 | 85.914 | 22.305 | 1.00 | 29.02 |
| 844 | CE1 | TYR | A | 446 | 11.589 | 87.417 | 21.204 | 1.00 | 33.24 |
| 845 | CE2 | TYR | A | 446 | 13.543 | 87.274 | 22.616 | 1.00 | 31.33 |
| 846 | CZ | TYR | A | 446 | 12.506 | 88.015 | 22.059 | 1.00 | 36.01 |
| 847 | OH | TYR | A | 446 | 12.387 | 89.352 | 22.338 | 1.00 | 36.54 |
| 848 | N | ALA | A | 447 | 13.914 | 85.326 | 18.060 | 1.00 | 28.07 |
| 849 | CA | ALA | A | 447 | 14.949 | 86.150 | 17.439 | 1.00 | 27.67 |
| 850 | C | ALA | A | 447 | 14.815 | 87.579 | 17.932 | 1.00 | 29.19 |
| 851 | O | ALA | A | 447 | 13.702 | 88.036 | 18.173 | 1.00 | 31.10 |
| 852 | CB | ALA | A | 447 | 14.802 | 86.098 | 15.931 | 1.00 | 23.97 |
| 853 | N | PHE | A | 448 | 15.929 | 88.293 | 18.079 | 1.00 | 29.56 |
| 854 | CA | PHE | A | 448 | 15.849 | 89.657 | 18.578 | 1.00 | 32.50 |
| 855 | C | PHE | A | 448 | 17.075 | 90.483 | 18.246 | 1.00 | 33.67 |
| 856 | O | PHE | A | 448 | 18.112 | 89.950 | 17.872 | 1.00 | 35.26 |
| 857 | CB | PHE | A | 448 | 15.659 | 89.630 | 20.097 | 1.00 | 35.29 |
| 858 | CG | PHE | A | 448 | 16.849 | 89.077 | 20.838 | 1.00 | 37.04 |
| 859 | CD1 | PHE | A | 448 | 17.912 | 89.909 | 21.195 | 1.00 | 36.66 |
| 860 | CD2 | PHE | A | 448 | 16.934 | 87.714 | 21.127 | 1.00 | 34.95 |
| 861 | CE1 | PHE | A | 448 | 19.049 | 89.382 | 21.824 | 1.00 | 40.91 |
| 862 | CE2 | PHE | A | 448 | 18.066 | 87.178 | 21.753 | 1.00 | 35.99 |
| 863 | CZ | PHE | A | 448 | 19.123 | 88.014 | 22.103 | 1.00 | 35.15 |
| 864 | N | ALA | A | 449 | 16.952 | 91.796 | 18.396 | 1.00 | 35.81 |
| 865 | CA | ALA | A | 449 | 18.066 | 92.693 | 18.131 | 1.00 | 37.63 |
| 866 | C | ALA | A | 449 | 18.617 | 93.253 | 19.438 | 1.00 | 40.96 |
| 867 | O | ALA | A | 449 | 17.885 | 93.441 | 20.403 | 1.00 | 40.14 |
| 868 | CB | ALA | A | 449 | 17.623 | 93.831 | 17.230 | 1.00 | 33.60 |
| 869 | N | THR | A | 450 | 19.918 | 93.509 | 19.448 | 1.00 | 45.40 |
| 870 | CA | THR | A | 450 | 20.606 | 94.071 | 20.595 | 1.00 | 50.82 |
| 871 | C | THR | A | 450 | 20.515 | 95.597 | 20.498 | 1.00 | 53.78 |
| 872 | O | THR | A | 450 | 20.673 | 96.169 | 19.417 | 1.00 | 54.14 |
| 873 | CB | THR | A | 450 | 22.090 | 93.665 | 20.587 | 1.00 | 51.45 |
| 874 | OG1 | THR | A | 450 | 22.192 | 92.242 | 20.450 | 1.00 | 54.12 |
| 875 | CG2 | THR | A | 450 | 22.769 | 94.093 | 21.877 | 1.00 | 55.58 |
| 876 | N | PRO | A | 451 | 20.260 | 96.275 | 21.626 | 1.00 | 56.74 |
| 877 | CA | PRO | A | 451 | 20.157 | 97.737 | 21.623 | 1.00 | 59.05 |
| 878 | C | PRO | A | 451 | 21.427 | 98.391 | 21.080 | 1.00 | 61.00 |
| 879 | O | PRO | A | 451 | 22.535 | 98.080 | 21.521 | 1.00 | 59.71 |
| 880 | CB | PRO | A | 451 | 19.933 | 98.073 | 23.098 | 1.00 | 60.52 |
| 881 | CC | PRO | A | 451 | 19.262 | 96.851 | 23.640 | 1.00 | 61.14 |
| 882 | CD | PRO | A | 451 | 20.049 | 95.739 | 22.981 | 1.00 | 59.39 |
| 883 | N | GLU | A | 452 | 21.272 | 99.285 | 20.112 | 1.00 | 64.30 |
| 884 | CA | GLU | A | 452 | 22.428 | 99.975 | 19.558 | 1.00 | 69.17 |
| 885 | C | GLU | A | 452 | 22.252 | 101.463 | 19.842 | 1.00 | 72.61 |
| 886 | O | GLU | A | 452 | 21.667 | 102.206 | 19.057 | 1.00 | 73.59 |
| 887 | CB | GLU | A | 452 | 22.560 | 99.712 | 18.054 | 1.00 | 65.66 |
| 888 | CG | GLU | A | 452 | 24.015 | 99.566 | 17.577 | 1.00 | 61.46 |
| 889 | CD | GLU | A | 452 | 24.717 | 98.317 | 18.133 | 1.00 | 60.52 |
| 890 | OE1 | GLU | A | 452 | 24.053 | 97.264 | 18.251 | 1.00 | 63.47 |
| 891 | OE2 | GLU | A | 452 | 25.934 | 98.373 | 18.433 | 1.00 | 53.34 |
| 892 | N | TRP | A | 453 | 22.763 | 101.869 | 20.997 | 1.00 | 76.75 |
| 893 | CA | TRP | A | 453 | 22.693 | 103.242 | 21.481 | 1.00 | 81.00 |
| 894 | C | TRP | A | 453 | 23.886 | 104.027 | 20.913 | 1.00 | 82.30 |
| 895 | O | TRP | A | 453 | 25.038 | 103.710 | 21.215 | 1.00 | 82.08 |
| 896 | CB | TRP | A | 453 | 22.737 | 103.191 | 23.010 | 1.00 | 85.65 |
| 897 | CG | TRP | A | 453 | 22.182 | 104.367 | 23.734 | 1.00 | 89.76 |
| 898 | CD1 | TRP | A | 453 | 20.939 | 104.921 | 23.588 | 1.00 | 92.34 |
| 899 | CD2 | TRP | A | 453 | 22.819 | 105.083 | 24.795 | 1.00 | 94.15 |
| 900 | NE1 | TRP | A | 453 | 20.764 | 105.934 | 24.502 | 1.00 | 94.99 |
| 901 | CE2 | TRP | A | 453 | 21.904 | 106.055 | 25.255 | 1.00 | 95.56 |
| 902 | CE3 | TRP | A | 453 | 24.079 | 104.995 | 25.407 | 1.00 | 95.36 |
| 903 | CZ2 | TRP | A | 453 | 22.208 | 106.935 | 26.300 | 1.00 | 97.14 |
| 904 | CZ3 | TRP | A | 453 | 24.380 | 105.869 | 26.445 | 1.00 | 95.61 |
| 905 | CH2 | TRP | A | 453 | 23.447 | 106.826 | 26.880 | 1.00 | 97.12 |
| 906 | N | PRO | A | 454 | 23.619 | 105.058 | 20.080 | 1.00 | 84.10 |
| 907 | CA | PRO | A | 454 | 24.606 | 105.931 | 19.422 | 1.00 | 87.24 |
| 908 | C | PRO | A | 454 | 25.892 | 106.316 | 20.162 | 1.00 | 90.52 |
| 909 | O | PRO | A | 454 | 26.088 | 105.978 | 21.333 | 1.00 | 89.75 |
| 910 | CB | PRO | A | 454 | 23.775 | 107.150 | 18.988 | 1.00 | 84.91 |
| 911 | CG | PRO | A | 454 | 22.475 | 107.017 | 19.740 | 1.00 | 83.62 |
| 912 | CD | PRO | A | 454 | 22.259 | 105.537 | 19.793 | 1.00 | 84.60 |
| 913 | N | GLY | A | 455 | 26.751 | 107.055 | 19.464 | 1.00 | 91.72 |
| 914 | CA | GLY | A | 455 | 28.048 | 107.413 | 20.012 | 1.00 | 94.13 |
| 915 | C | GLY | A | 455 | 28.730 | 106.132 | 19.601 | 1.00 | 96.28 |
| 916 | O | GLY | A | 455 | 28.613 | 105.125 | 20.301 | 1.00 | 97.74 |
| 917 | N | SER | A | 456 | 29.442 | 106.142 | 18.478 | 1.00 | 99.11 |
| 918 | CA | SER | A | 456 | 29.974 | 104.869 | 18.023 | 1.00 | 96.15 |
| 919 | C | SER | A | 456 | 31.407 | 104.465 | 17.676 | 1.00 | 91.50 |
| 920 | O | SER | A | 456 | 32.292 | 105.284 | 17.333 | 1.00 | 88.52 |
| 921 | CB | SER | A | 456 | 29.092 | 104.378 | 16.879 | 1.00 | 95.05 |
| 922 | OG | SER | A | 456 | 28.910 | 105.375 | 15.884 | 1.00 | 110.76 |
| 923 | N | ARG | A | 457 | 31.541 | 103.136 | 17.752 | 1.00 | 87.73 |
| 924 | CA | ARG | A | 457 | 32.664 | 102.251 | 17.520 | 1.00 | 87.81 |
| 925 | C | ARG | A | 457 | 31.678 | 101.103 | 17.647 | 1.00 | 86.30 |
| 926 | O | ARG | A | 457 | 31.026 | 100.998 | 18.697 | 1.00 | 90.57 |
| 927 | CB | ARG | A | 457 | 33.634 | 102.261 | 18.675 | 1.00 | 94.67 |
| 928 | N | ASP | A | 458 | 31.512 | 100.259 | 16.627 | 1.00 | 87.32 |
| 929 | CA | ASP | A | 458 | 30.441 | 99.286 | 16.780 | 1.00 | 80.43 |
| 930 | C | ASP | A | 458 | 30.422 | 97.963 | 16.080 | 1.00 | 72.83 |
| 931 | O | ASP | A | 458 | 31.410 | 97.437 | 15.599 | 1.00 | 73.96 |
| 932 | CB | ASP | A | 458 | 29.150 | 99.985 | 16.385 | 1.00 | 83.19 |
| 933 | CG | ASP | A | 458 | 29.102 | 100.300 | 14.890 | 1.00 | 85.09 |
| 934 | OD1 | ASP | A | 458 | 30.065 | 100.906 | 14.378 | 1.00 | 91.25 |
| 935 | OD2 | ASP | A | 458 | 28.108 | 99.939 | 14.217 | 1.00 | 90.84 |
| 936 | N | LYS | A | 459 | 29.196 | 97.451 | 16.076 | 1.00 | 66.05 |
| 937 | CA | LYS | A | 459 | 28.782 | 96.211 | 15.437 | 1.00 | 61.75 |
| 938 | C | LYS | A | 459 | 27.368 | 95.983 | 15.886 | 1.00 | 56.14 |
| 939 | O | LYS | A | 459 | 27.144 | 95.648 | 17.046 | 1.00 | 56.54 |
| 940 | CB | LYS | A | 459 | 29.641 | 95.031 | 15.889 | 1.00 | 68.18 |
| 941 | CG | LYS | A | 459 | 30.801 | 94.804 | 14.969 | 1.00 | 71.85 |
| 942 | CD | LYS | A | 459 | 30.406 | 94.863 | 13.499 | 1.00 | 73.79 |
| 943 | CE | LYS | A | 459 | 31.655 | 94.839 | 12.626 | 1.00 | 76.14 |
| 944 | NZ | LYS | A | 459 | 31.341 | 94.675 | 11.169 | 1.00 | 79.35 |
| 945 | N | ARG | A | 460 | 26.406 | 96.194 | 15.001 | 1.00 | 52.32 |
| 946 | CA | ARG | A | 460 | 25.035 | 95.951 | 15.377 | 1.00 | 47.59 |
| 947 | C | ARG | A | 460 | 24.860 | 94.436 | 15.290 | 1.00 | 43.34 |
| 948 | O | ARG | A | 460 | 25.477 | 93.761 | 14.462 | 1.00 | 40.40 |
| 949 | CB | ARG | A | 460 | 24.104 | 96.726 | 14.456 | 1.00 | 49.27 |
| 950 | CG | ARG | A | 460 | 24.275 | 98.216 | 14.671 | 1.00 | 56.23 |
| 951 | CD | ARG | A | 460 | 23.659 | 99.006 | 13.532 | 1.00 | 63.20 |
| 952 | NE | ARG | A | 460 | 23.997 | 100.426 | 13.596 | 1.00 | 68.02 |
| 953 | CZ | ARG | A | 460 | 25.145 | 100.955 | 13.180 | 1.00 | 70.46 |
| 954 | NH1 | ARG | A | 460 | 26.094 | 100.185 | 12.656 | 1.00 | 72.23 |
| 955 | NH2 | ARG | A | 460 | 25.336 | 102.264 | 13.285 | 1.00 | 73.53 |
| 956 | N | THR | A | 461 | 24.062 | 93.910 | 16.198 | 1.00 | 39.19 |
| 957 | CA | THR | A | 461 | 23.879 | 92.474 | 16.278 | 1.00 | 36.81 |
| 958 | C | THR | A | 461 | 22.447 | 92.004 | 16.401 | 1.00 | 34.03 |
| 959 | O | THR | A | 461 | 21.631 | 92.607 | 17.110 | 1.00 | 33.96 |
| 960 | CB | THR | A | 461 | 24.603 | 91.890 | 17.512 | 1.00 | 39.13 |
| 961 | OG1 | THR | A | 461 | 25.957 | 92.357 | 17.557 | 1.00 | 46.04 |
| 962 | CG2 | THR | A | 461 | 24.630 | 90.380 | 17.421 | 1.00 | 45.48 |
| 963 | N | LEU | A | 462 | 22.157 | 90.904 | 15.714 | 1.00 | 29.84 |
| 964 | CA | LEU | A | 462 | 20.853 | 90.267 | 15.788 | 1.00 | 28.03 |
| 965 | C | LEU | A | 462 | 21.205 | 88.926 | 16.410 | 1.00 | 27.33 |
| 966 | O | LEU | A | 462 | 22.307 | 88.414 | 16.207 | 1.00 | 24.47 |
| 967 | CB | LEU | A | 462 | 20.223 | 90.074 | 14.403 | 1.00 | 25.97 |
| 968 | CG | LEU | A | 462 | 19.888 | 91.362 | 13.635 | 1.00 | 33.39 |
| 969 | CO1 | LEU | A | 462 | 19.172 | 91.007 | 12.319 | 1.00 | 32.36 |
| 970 | CD2 | LEU | A | 462 | 19.014 | 92.263 | 14.483 | 1.00 | 30.72 |
| 971 | N | ALA | A | 463 | 20.286 | 88.359 | 17.178 | 1.00 | 25.83 |
| 972 | CA | ALA | A | 463 | 20.577 | 87.111 | 17.836 | 1.00 | 25.65 |
| 973 | C | ALA | A | 463 | 19.330 | 86.279 | 17.957 | 1.00 | 26.32 |
| 974 | O | ALA | A | 463 | 18.214 | 86.790 | 17.846 | 1.00 | 27.53 |
| 975 | CB | ALA | A | 463 | 21.179 | 87.381 | 19.212 | 1.00 | 28.15 |
| 976 | N | CYS | A | 464 | 19.518 | 84.992 | 18.195 | 1.00 | 21.23 |
| 977 | CA | CYS | A | 464 | 18.388 | 84.093 | 18.276 | 1.00 | 24.77 |
| 978 | C | CYS | A | 464 | 18.599 | 83.016 | 19.314 | 1.00 | 22.14 |
| 979 | O | CYS | A | 464 | 19.683 | 82.431 | 19.402 | 1.00 | 24.66 |
| 980 | CB | CYS | A | 464 | 18.193 | 83.429 | 16.914 | 1.00 | 22.04 |
| 981 | SG | CYS | A | 464 | 16.790 | 82.298 | 16.720 | 1.00 | 34.06 |
| 982 | N | LEU | A | 465 | 17.548 | 82.741 | 20.075 | 1.00 | 22.44 |
| 983 | CA | LEU | A | 465 | 17.598 | 81.697 | 21.089 | 1.00 | 23.11 |
| 984 | C | LEU | A | 465 | 16.644 | 80.615 | 20.641 | 1.00 | 22.43 |
| 985 | O | LEU | A | 465 | 15.483 | 80.898 | 20.352 | 1.00 | 23.73 |
| 986 | CB | LEU | A | 465 | 17.155 | 82.230 | 22.461 | 1.00 | 22.99 |
| 987 | CG | LEU | A | 465 | 16.810 | 81.177 | 23.538 | 1.00 | 19.04 |
| 988 | CD1 | LEU | A | 465 | 18.035 | 80.331 | 23.866 | 1.00 | 21.72 |
| 989 | CD2 | LEU | A | 465 | 16.347 | 81.884 | 24.818 | 1.00 | 28.23 |
| 990 | N | ILE | A | 466 | 17.125 | 79.380 | 20.581 | 1.00 | 22.56 |
| 991 | CA | ILE | A | 466 | 16.277 | 78.268 | 20.169 | 1.00 | 22.11 |
| 992 | C | ILE | A | 466 | 16.302 | 77.256 | 21.297 | 1.00 | 24.25 |
| 993 | O | ILE | A | 466 | 17.375 | 76.815 | 21.706 | 1.00 | 24.93 |
| 994 | CB | ILE | A | 466 | 16.781 | 77.654 | 18.861 | 1.00 | 22.15 |
| 995 | CG1 | ILE | A | 466 | 16.849 | 78.750 | 17.786 | 1.00 | 23.07 |
| 996 | CG2 | ILE | A | 466 | 15.822 | 76.525 | 18.416 | 1.00 | 25.24 |
| 997 | CD1 | ILE | A | 466 | 17.509 | 78.318 | 16.496 | 1.00 | 33.12 |
| 998 | N | GLN | A | 467 | 15.132 | 76.863 | 21.793 | 1.00 | 24.63 |
| 999 | CA | GLN | A | 467 | 15.120 | 75.970 | 22.951 | 1.00 | 28.64 |
| 1000 | C | GLN | A | 467 | 13.982 | 74.963 | 23.072 | 1.00 | 31.08 |
| 1001 | O | GLN | A | 467 | 13.071 | 74.915 | 22.235 | 1.00 | 31.00 |
| 1002 | CB | GLN | A | 467 | 15.172 | 76.830 | 24.228 | 1.00 | 25.58 |
| 1003 | CG | GLN | A | 467 | 13.943 | 77.727 | 24.404 | 1.00 | 25.53 |
| 1004 | CD | GLN | A | 467 | 14.044 | 78.693 | 25.599 | 1.00 | 30.72 |
| 1005 | OE1 | GLN | A | 467 | 14.790 | 78.456 | 26.549 | 1.00 | 29.76 |
| 1006 | NE2 | GLN | A | 467 | 13.273 | 79.779 | 25.550 | 1.00 | 32.18 |
| 1007 | N | ASN | A | 468 | 14.083 | 74.147 | 24.123 | 1.00 | 33.71 |
| 1008 | CA | ASN | A | 468 | 13.114 | 73.106 | 24.459 | 1.00 | 36.96 |
| 1009 | C | ASN | A | 468 | 12.993 | 71.999 | 23.423 | 1.00 | 38.20 |
| 1010 | O | ASN | A | 468 | 11.931 | 71.392 | 23.288 | 1.00 | 38.39 |
| 1011 | CB | ASN | A | 468 | 11.731 | 73.719 | 24.676 | 1.00 | 41.06 |
| 1012 | CG | ASN | A | 468 | 11.748 | 74.837 | 25.686 | 1.00 | 46.41 |
| 1013 | OD1 | ASN | A | 468 | 12.372 | 74.721 | 26.741 | 1.00 | 50.82 |
| 1014 | ND2 | ASN | A | 468 | 11.052 | 75.928 | 25.376 | 1.00 | 53.31 |
| 1015 | N | PHE | A | 469 | 14.065 | 71.732 | 22.685 | 1.00 | 35.74 |
| 1016 | CA | PHE | A | 469 | 14.003 | 70.690 | 21.677 | 1.00 | 33.87 |
| 1017 | C | PHE | A | 469 | 14.804 | 69.453 | 22.056 | 1.00 | 33.63 |
| 1018 | O | PHE | A | 469 | 15.727 | 69.513 | 22.860 | 1.00 | 34.78 |
| 1019 | CB | PHE | A | 469 | 14.495 | 71.228 | 20.322 | 1.00 | 29.12 |
| 1020 | CG | PHE | A | 469 | 15.926 | 71.691 | 20.334 | 1.00 | 26.27 |
| 1021 | CO1 | PHE | A | 469 | 16.959 | 70.792 | 20.133 | 1.00 | 22.45 |
| 1022 | CD2 | PHE | A | 469 | 16.238 | 73.034 | 20.539 | 1.00 | 22.06 |
| 1023 | CE1 | PHE | A | 469 | 18.285 | 71.214 | 20.126 | 1.00 | 23.84 |
| 1024 | CE2 | PHE | A | 469 | 17.564 | 73.465 | 20.536 | 1.00 | 28.02 |
| 1025 | CZ | PHE | A | 469 | 18.593 | 72.549 | 20.327 | 1.00 | 23.33 |
| 1026 | N | MET | A | 470 | 14.431 | 68.327 | 21.466 | 1.00 | 35.64 |
| 1027 | CA | MET | A | 470 | 15.120 | 67.065 | 21.687 | 1.00 | 37.62 |
| 1028 | C | MET | A | 470 | 14.684 | 66.104 | 20.596 | 1.00 | 36.36 |
| 1029 | O | MET | A | 470 | 13.529 | 66.122 | 20.177 | 1.00 | 36.33 |
| 1030 | CB | MET | A | 470 | 14.804 | 66.495 | 23.076 | 1.00 | 42.61 |
| 1031 | CG | MET | A | 470 | 13.354 | 66.179 | 23.350 | 1.00 | 50.60 |
| 1032 | SD | MET | A | 470 | 13.141 | 65.744 | 25.113 | 1.00 | 66.08 |
| 1033 | CE | MET | A | 470 | 13.780 | 64.056 | 25.134 | 1.00 | 66.35 |
| 1034 | N | PRO | A | 471 | 15.613 | 65.283 | 20.079 | 1.00 | 34.63 |
| 1035 | CA | PRO | A | 471 | 17.039 | 65.169 | 20.418 | 1.00 | 32.61 |
| 1036 | C | PRO | A | 471 | 17.818 | 66.453 | 20.143 | 1.00 | 31.92 |
| 1037 | O | PRO | A | 471 | 17.244 | 67.436 | 19.685 | 1.00 | 29.59 |
| 1038 | CB | PRO | A | 471 | 17.520 | 64.015 | 19.538 | 1.00 | 33.95 |
| 1039 | CG | PRO | A | 471 | 16.277 | 63.227 | 19.295 | 1.00 | 37.90 |
| 1040 | CD | PRO | A | 471 | 15.246 | 64.291 | 19.058 | 1.00 | 34.78 |
| 1041 | N | GLU | A | 472 | 19.128 | 66.413 | 20.386 | 1.00 | 32.28 |
| 1042 | CA | GLU | A | 472 | 20.001 | 67.580 | 20.234 | 1.00 | 36.49 |
| 1043 | C | GLU | A | 472 | 20.380 | 68.043 | 18.830 | 1.00 | 35.52 |
| 1044 | O | GLU | A | 472 | 20.870 | 69.162 | 18.683 | 1.00 | 34.87 |
| 1045 | CB | GLU | A | 472 | 21.302 | 67.377 | 21.015 | 1.00 | 41.97 |
| 1046 | CG | GLU | A | 472 | 22.213 | 66.306 | 20.423 | 1.00 | 54.78 |
| 1047 | CD | GLU | A | 472 | 23.527 | 66.144 | 21.184 | 1.00 | 62.41 |
| 1048 | OE1 | GLU | A | 472 | 23.603 | 66.579 | 22.356 | 1.00 | 67.26 |
| 1O49 | OE2 | GLU | A | 472 | 24.481 | 65.566 | 20.613 | 1.00 | 64.96 |
| 1050 | N | ASP | A | 473 | 20.161 | 67.221 | 17.805 | 1.00 | 34.38 |
| 1051 | CA | ASP | A | 473 | 20.543 | 67.629 | 16.452 | 1.00 | 31.83 |
| 1052 | C | ASP | A | 473 | 19.602 | 68.686 | 15.886 | 1.00 | 31.24 |
| 1053 | O | ASP | A | 473 | 18.380 | 68.528 | 15.901 | 1.00 | 31.80 |
| 1054 | CB | ASP | A | 473 | 20.621 | 66.420 | 15.516 | 1.00 | 34.73 |
| 1055 | CG | ASP | A | 473 | 21.746 | 65.453 | 15.904 | 1.00 | 40.83 |
| 1056 | OD1 | ASP | A | 473 | 22.841 | 65.924 | 16.296 | 1.00 | 44.18 |
| 1057 | OD2 | ASP | A | 473 | 21.539 | 64.222 | 15.810 | 1.00 | 40.17 |
| 1058 | N | ILE | A | 474 | 20.184 | 69.774 | 15.398 | 1.00 | 26.96 |
| 1059 | CA | ILE | A | 474 | 19.393 | 70.856 | 14.854 | 1.00 | 25.39 |
| 1060 | C | ILE | A | 474 | 20.225 | 71.715 | 13.892 | 1.00 | 24.74 |
| 1061 | O | ILE | A | 474 | 21.436 | 71.824 | 14.031 | 1.00 | 25.20 |
| 1062 | CB | ILE | A | 474 | 18.842 | 71.738 | 16.008 | 1.00 | 23.81 |
| 1063 | CG1 | ILE | A | 474 | 17.748 | 72.675 | 15.502 | 1.00 | 22.23 |
| 1064 | CG2 | ILE | A | 474 | 19.974 | 72.538 | 16.647 | 1.00 | 27.10 |
| 1065 | CO1 | ILE | A | 474 | 17.064 | 73.456 | 16.650 | 1.00 | 25.30 |
| 1066 | N | SER | A | 475 | 19.570 | 72.288 | 12.891 | 1.00 | 23.27 |
| 1067 | CA | SER | A | 475 | 20.252 | 73.170 | 11.952 | 1.00 | 22.17 |
| 1068 | C | SER | A | 475 | 19.609 | 74.534 | 12.148 | 1.00 | 21.71 |
| 1069 | O | SER | A | 475 | 18.393 | 74.649 | 12.224 | 1.00 | 20.84 |
| 1070 | CB | SER | A | 475 | 20.049 | 72.721 | 10.507 | 1.00 | 20.43 |
| 1071 | OG | SER | A | 475 | 20.589 | 71.436 | 10.294 | 1.00 | 30.00 |
| 1072 | N | VAL | A | 476 | 20.442 | 75.558 | 12.213 | 1.00 | 21.76 |
| 1073 | CA | VAL | A | 476 | 19.980 | 76.906 | 12.425 | 1.00 | 21.72 |
| 1074 | C | VAL | A | 476 | 20.435 | 77.725 | 11.249 | 1.00 | 23.56 |
| 1075 | O | VAL | A | 476 | 21.555 | 77.564 | 10.780 | 1.00 | 25.95 |
| 1076 | CB | VAL | A | 476 | 20.606 | 77.513 | 13.715 | 1.00 | 21.56 |
| 1077 | CO1 | VAL | A | 476 | 20.157 | 78.975 | 13.878 | 1.00 | 18.70 |
| 1078 | CG2 | VAL | A | 476 | 20.197 | 76.685 | 14.934 | 1.00 | 20.99 |
| 1079 | N | GLN | A | 477 | 19.574 | 78.606 | 10.764 | 1.00 | 25.16 |
| 1080 | CA | GLN | A | 477 | 19.965 | 79.428 | 9.640 | 1.00 | 26.65 |
| 1081 | C | GLN | A | 477 | 19.263 | 80.767 | 9.662 | 1.00 | 26.62 |
| 1082 | O | GLN | A | 477 | 18.160 | 80.891 | 10.175 | 1.00 | 26.40 |
| 1083 | CB | GLN | A | 477 | 19.701 | 78.683 | 8.325 | 1.00 | 31.51 |
| 1084 | CG | GLN | A | 477 | 18.289 | 78.252 | 8.089 | 1.00 | 44.27 |
| 1085 | CD | GLN | A | 477 | 18.188 | 77.095 | 7.084 | 1.00 | 47.76 |
| 1086 | DE1 | GLN | A | 477 | 17.104 | 76.786 | 6.598 | 1.00 | 50.57 |
| 1087 | NE2 | GLN | A | 477 | 19.317 | 76.449 | 6.787 | 1.00 | 47.32 |
| 1088 | N | TRP | A | 478 | 19.941 | 81.780 | 9.146 | 1.00 | 27.41 |
| 1089 | CA | TRP | A | 478 | 19.375 | 83.112 | 9.074 | 1.00 | 28.36 |
| 1090 | C | TRP | A | 478 | 18.944 | 83.408 | 7.635 | 1.00 | 30.10 |
| 1091 | O | TRP | A | 478 | 19.567 | 82.940 | 6.672 | 1.00 | 25.31 |
| 1092 | CB | TRP | A | 478 | 20.404 | 84.139 | 9.533 | 1.00 | 28.76 |
| 1093 | CG | TRP | A | 478 | 20.649 | 84.098 | 11.013 | 1.00 | 34.11 |
| 1094 | CD1 | TRP | A | 478 | 21.522 | 83.287 | 11.685 | 1.00 | 31.25 |
| 1095 | CD2 | TRP | A | 478 | 19.980 | 84.878 | 12.006 | 1.00 | 31.01 |
| 1096 | NE1 | TRP | A | 478 | 21.436 | 83.515 | 13.031 | 1.00 | 27.71 |
| 1097 | CE2 | TRP | A | 478 | 20.496 | 84.487 | 13.259 | 1.00 | 31.95 |
| 1098 | CE3 | TRP | A | 478 | 18.993 | 85.871 | 11.960 | 1.00 | 34.16 |
| 1099 | CZ2 | TRP | A | 478 | 20.055 | 85.058 | 14.463 | 1.00 | 25.26 |
| 1100 | CZ3 | TRP | A | 478 | 18.556 | 86.439 | 13.153 | 1.00 | 31.64 |
| 1101 | CH2 | TRP | A | 478 | 19.089 | 86.029 | 14.387 | 1.00 | 28.89 |
| 1102 | N | LEU | A | 479 | 17.877 | 84.182 | 7.498 | 1.00 | 32.48 |
| 1103 | CA | LEU | A | 479 | 17.355 | 84.551 | 6.187 | 1.00 | 37.36 |
| 1104 | C | LEU | A | 479 | 16.943 | 86.016 | 6.140 | 1.00 | 40.42 |
| 1105 | O | LEU | A | 479 | 16.448 | 86.560 | 7.123 | 1.00 | 40.14 |
| 1106 | CE | LEU | A | 479 | 16.122 | 83.722 | 5.848 | 1.00 | 39.83 |
| 1107 | CG | LEU | A | 479 | 16.044 | 82.254 | 6.258 | 1.00 | 44.57 |
| 1108 | CD1 | LEU | A | 479 | 14.713 | 81.710 | 5.810 | 1.00 | 52.98 |
| 1109 | CD2 | LEU | A | 479 | 17.161 | 81.470 | 5.634 | 1.00 | 50.71 |
| 1110 | N | HIS | A | 480 | 17.152 | 86.653 | 4.998 | 1.00 | 43.05 |
| 1111 | CA | HIS | A | 480 | 16.723 | 88.028 | 4.824 | 1.00 | 49.20 |
| 1112 | C | HIS | A | 480 | 15.464 | 87.854 | 3.984 | 1.00 | 52.73 |
| 1113 | O | HIS | A | 480 | 15.509 | 87.895 | 2.753 | 1.00 | 52.90 |
| 1114 | CB | HIS | A | 480 | 17.756 | 88.846 | 4.057 | 1.00 | 52.25 |
| 1115 | CD | HIS | A | 480 | 17.330 | 90.259 | 3.814 | 1.00 | 57.22 |
| 1116 | ND1 | HIS | A | 480 | 17.525 | 91.263 | 4.736 | 1.00 | 61.37 |
| 1117 | CD2 | HIS | A | 480 | 16.658 | 90.822 | 2.781 | 1.00 | 59.16 |
| 1118 | CE1 | HIS | A | 480 | 16.993 | 92.384 | 4.285 | 1.00 | 59.88 |
| 1119 | NE2 | HIS | A | 480 | 16.460 | 92.144 | 3.100 | 1.00 | 66.11 |
| 1120 | N | ASN | A | 481 | 14.347 | 87.629 | 4.670 | 1.00 | 56.65 |
| 1121 | CA | ASN | A | 481 | 13.057 | 87.394 | 4.030 | 1.00 | 59.93 |
| 1122 | C | ASN | A | 481 | 13.009 | 85.940 | 3.581 | 1.00 | 59.65 |
| 1123 | O | ASN | A | 481 | 13.031 | 85.029 | 4.405 | 1.00 | 61.04 |
| 1124 | CB | ASN | A | 481 | 12.849 | 88.312 | 2.818 | 1.00 | 64.65 |
| 1125 | CG | ASN | A | 481 | 12.578 | 89.748 | 3.210 | 1.00 | 72.38 |
| 1126 | OD1 | ASN | A | 481 | 11.636 | 90.037 | 3.953 | 1.00 | 74.96 |
| 1127 | ND2 | ASN | A | 481 | 13.401 | 90.662 | 2.707 | 1.00 | 75.60 |
| 1128 | N | GLU | A | 482 | 12.975 | 85.728 | 2.273 | 1.00 | 58.97 |
| 1129 | CA | GLU | A | 482 | 12.903 | 84.387 | 1.704 | 1.00 | 58.55 |
| 1130 | C | GLU | A | 482 | 14.254 | 83.785 | 1.318 | 1.00 | 55.30 |
| 1131 | O | GLU | A | 482 | 14.347 | 82.587 | 1.056 | 1.00 | 57.27 |
| 1132 | CE | GLU | A | 482 | 11.993 | 84.424 | 0.473 | 1.00 | 65.42 |
| 1133 | CD | GLU | A | 482 | 11.787 | 85.840 | 0.072 | 1.00 | 73.94 |
| 1134 | CD | GLU | A | 482 | 11.127 | 85.865 | 1.432 | 1.00 | 80.29 |
| 1135 | OE1 | GLU | A | 482 | 11.770 | 85.432 | 2.413 | 1.00 | 86.31 |
| 1136 | OE2 | GLU | A | 482 | 9.967 | 86.316 | 1.520 | 1.00 | 82.73 |
| 1137 | N | VAL | A | 483 | 15.299 | 84.603 | 1.286 | 1.00 | 50.25 |
| 1138 | CA | VAL | A | 483 | 16.612 | 84.113 | 0.889 | 1.00 | 45.78 |
| 1139 | C | VAL | A | 483 | 17.575 | 83.867 | 2.048 | 1.00 | 43.04 |
| 1140 | O | VAL | A | 483 | 17.766 | 84.720 | 2.902 | 1.00 | 41.34 |
| 1141 | CE | VAL | A | 483 | 17.261 | 85.073 | 0.133 | 1.00 | 43.76 |
| 1142 | CG1 | VAL | A | 483 | 17.317 | 86.471 | 0.432 | 1.00 | 46.25 |
| 1143 | CG2 | VAL | A | 483 | 18.654 | 84.589 | 0.494 | 1.00 | 41.37 |
| 1144 | N | GLN | A | 484 | 18.188 | 82.688 | 2.050 | 1.00 | 42.30 |
| 1145 | CA | GLN | A | 484 | 19.126 | 82.300 | 3.095 | 1.00 | 43.21 |
| 1146 | C | GLN | A | 484 | 20.509 | 82.942 | 2.959 | 1.00 | 42.48 |
| 1147 | O | GLN | A | 484 | 21.055 | 83.062 | 1.856 | 1.00 | 41.64 |
| 1148 | CE | GLN | A | 484 | 19.269 | 80.774 | 3.129 | 1.00 | 45.96 |
| 1149 | CG | GLN | A | 484 | 20.331 | 80.276 | 4.101 | 1.00 | 54.43 |
| 1150 | CD | GLN | A | 484 | 20.427 | 78.760 | 4.159 | 1.00 | 59.02 |
| 1151 | OE1 | GLN | A | 484 | 21.400 | 78.209 | 4.677 | 1.00 | 59.71 |
| 1152 | NE2 | GLN | A | 484 | 19.410 | 78.078 | 3.636 | 1.00 | 61.46 |
| 1153 | N | LEU | A | 485 | 21.061 | 83.361 | 4.094 | 1.00 | 38.99 |
| 1154 | CA | LEU | A | 485 | 22.382 | 83.982 | 4.147 | 1.00 | 39.17 |
| 1155 | C | LEU | A | 485 | 23.458 | 82.900 | 4.247 | 1.00 | 40.35 |
| 1156 | O | LEU | A | 485 | 23.183 | 81.778 | 4.670 | 1.00 | 37.39 |
| 1157 | CE | LEU | A | 485 | 22.493 | 84.895 | 5.375 | 1.00 | 37.16 |
| 1158 | CD | LEU | A | 485 | 21.460 | 86.021 | 5.498 | 1.00 | 39.66 |
| 1159 | CD1 | LEU | A | 485 | 21.670 | 86.764 | 6.807 | 1.00 | 39.30 |
| 1160 | CD2 | LEU | A | 485 | 21.580 | 86.956 | 4.305 | 1.00 | 40.56 |
| 1161 | N | PRO | A | 486 | 24.700 | 83.227 | 3.858 | 1.00 | 42.55 |
| 1162 | CA | PRO | A | 486 | 25.772 | 82.234 | 3.942 | 1.00 | 46.30 |
| 1163 | C | PRO | A | 486 | 25.940 | 81.850 | 5.412 | 1.00 | 49.32 |
| 1164 | O | PRO | A | 486 | 25.748 | 82.684 | 6.294 | 1.00 | 48.81 |
| 1165 | CB | PRO | A | 486 | 26.991 | 82.994 | 3.425 | 1.00 | 45.64 |
| 1166 | CG | PRO | A | 486 | 26.413 | 84.068 | 2.572 | 1.00 | 45.29 |
| 1167 | CD | PRO | A | 486 | 25.206 | 84.505 | 3.331 | 1.00 | 40.65 |
| 1168 | N | ASP | A | 487 | 26.310 | 80.603 | 5.671 | 1.00 | 53.39 |
| 1169 | CA | ASP | A | 487 | 26.507 | 80.135 | 7.038 | 1.00 | 57.75 |
| 1170 | C | ASP | A | 487 | 27.603 | 80.940 | 7.750 | 1.00 | 57.25 |
| 1171 | O | ASP | A | 487 | 27.424 | 81.395 | 8.887 | 1.00 | 57.75 |
| 1172 | CB | ASP | A | 487 | 26.892 | 78.656 | 7.023 | 1.00 | 67.82 |
| 1173 | CG | ASP | A | 487 | 26.821 | 78.023 | 8.397 | 1.00 | 76.17 |
| 1174 | OD1 | ASP | A | 487 | 27.291 | 78.655 | 9.366 | 1.00 | 84.77 |
| 1175 | OD2 | ASP | A | 487 | 26.303 | 76.889 | 8.508 | 1.00 | 86.94 |
| 1176 | N | ALA | A | 488 | 28.727 | 81.122 | 7.065 | 1.00 | 54.64 |
| 1177 | CA | ALA | A | 488 | 29.871 | 81.848 | 7.607 | 1.00 | 53.45 |
| 1178 | C | ALA | A | 488 | 29.531 | 83.179 | 8.263 | 1.00 | 52.74 |
| 1179 | O | ALA | A | 488 | 30.359 | 83.757 | 8.966 | 1.00 | 53.06 |
| 1180 | CB | ALA | A | 488 | 30.892 | 82.070 | 6.515 | 1.00 | 53.75 |
| 1181 | N | ARG | A | 489 | 28.313 | 83.662 | 8.044 | 1.00 | 50.96 |
| 1182 | CA | ARG | A | 489 | 27.896 | 84.940 | 8.609 | 1.00 | 47.84 |
| 1183 | C | ARG | A | 489 | 27.436 | 84.895 | 10.064 | 1.00 | 45.10 |
| 1184 | O | ARG | A | 489 | 27.442 | 85.919 | 10.737 | 1.00 | 43.37 |
| 1185 | CB | ARG | A | 489 | 26.792 | 85.554 | 7.746 | 1.00 | 48.99 |
| 1186 | CG | ARG | A | 489 | 27.306 | 86.368 | 6.576 | 1.00 | 50.01 |
| 1187 | CD | ARG | A | 489 | 27.173 | 87.839 | 6.873 | 1.00 | 50.36 |
| 1188 | NE | ARG | A | 489 | 26.019 | 88.413 | 6.195 | 1.00 | 56.65 |
| 1189 | CZ | ARG | A | 489 | 25.434 | 89.552 | 6.543 | 1.00 | 58.23 |
| 1190 | NH1 | ARG | A | 489 | 25.886 | 90.248 | 7.578 | 1.00 | 61.07 |
| 1191 | NH2 | ARG | A | 489 | 24.407 | 90.006 | 5.840 | 1.00 | 59.63 |
| 1192 | N | HIS | A | 490 | 27.027 | 83.728 | 10.549 | 1.00 | 42.41 |
| 1193 | CA | HIS | A | 490 | 26.576 | 83.635 | 11.934 | 1.00 | 41.63 |
| 1194 | C | HIS | A | 490 | 27.390 | 82.648 | 12.766 | 1.00 | 39.71 |
| 1195 | O | HIS | A | 490 | 28.129 | 81.832 | 12.235 | 1.00 | 41.30 |
| 1196 | CB | HIS | A | 490 | 25.092 | 83.252 | 12.000 | 1.00 | 42.40 |
| 1197 | CC | HIS | A | 490 | 24.793 | 81.880 | 11.486 | 1.00 | 43.27 |
| 1198 | ND1 | HIS | A | 490 | 24.423 | 81.634 | 10.181 | 1.00 | 42.27 |
| 1199 | CD2 | HIS | A | 490 | 24.821 | 80.674 | 12.101 | 1.00 | 44.41 |
| 1200 | CE1 | HIS | A | 490 | 24.236 | 80.338 | 10.014 | 1.00 | 39.88 |
| 1201 | NE2 | HIS | A | 490 | 24.470 | 79.732 | 11.166 | 1.00 | 45.37 |
| 1202 | N | SER | A | 491 | 27.245 | 82.742 | 14.080 | 1.00 | 37.20 |
| 1203 | CA | SER | A | 491 | 27.946 | 81.866 | 15.005 | 1.00 | 36.36 |
| 1204 | C | SER | A | 491 | 26.915 | 81.199 | 15.927 | 1.00 | 33.06 |
| 1205 | O | SER | A | 491 | 26.172 | 81.879 | 16.639 | 1.00 | 33.77 |
| 1206 | CB | SER | A | 491 | 28.949 | 82.690 | 15.812 | 1.00 | 35.42 |
| 1207 | OC | SER | A | 491 | 29.547 | 81.906 | 16.818 | 1.00 | 43.65 |
| 1208 | N | THR | A | 492 | 26.878 | 79.872 | 15.912 | 1.00 | 29.90 |
| 1209 | CA | THR | A | 492 | 25.927 | 79.105 | 16.717 | 1.00 | 29.91 |
| 1210 | C | THR | A | 492 | 26.623 | 78.278 | 17.813 | 1.00 | 29.20 |
| 1211 | O | THR | A | 492 | 27.609 | 77.604 | 17.548 | 1.00 | 30.85 |
| 1212 | CE | THR | A | 492 | 25.094 | 78.153 | 15.802 | 1.00 | 29.85 |
| 1213 | OC1 | THR | A | 492 | 24.403 | 78.930 | 14.819 | 1.00 | 35.86 |
| 1214 | CC2 | THR | A | 492 | 24.059 | 77.366 | 16.602 | 1.00 | 28.05 |
| 1215 | N | THR | A | 493 | 26.095 | 78.319 | 19.035 | 1.00 | 29.49 |
| 1216 | CA | THR | A | 493 | 26.687 | 77.568 | 20.143 | 1.00 | 30.19 |
| 1217 | C | THR | A | 493 | 26.305 | 76.099 | 20.044 | 1.00 | 32.39 |
| 1218 | O | THR | A | 493 | 25.390 | 75.737 | 19.314 | 1.00 | 30.70 |
| 1219 | CB | THR | A | 493 | 26.222 | 78.098 | 21.532 | 1.00 | 27.82 |
| 1220 | OC1 | THR | A | 493 | 24.794 | 78.008 | 21.640 | 1.00 | 30.48 |
| 1221 | CC2 | THR | A | 493 | 26.647 | 79.533 | 21.728 | 1.00 | 22.03 |
| 1222 | N | GLN | A | 494 | 27.014 | 75.253 | 20.778 | 1.00 | 35.09 |
| 1223 | CA | GLN | A | 494 | 26.721 | 73.821 | 20.770 | 1.00 | 38.19 |
| 1224 | C | GLN | A | 494 | 25.509 | 73.568 | 21.651 | 1.00 | 36.48 |
| 1225 | O | GLN | A | 494 | 25.330 | 74.240 | 22.668 | 1.00 | 35.75 |
| 1226 | CB | GLN | A | 494 | 27.904 | 73.019 | 21.325 | 1.00 | 43.09 |
| 1227 | CG | GLN | A | 494 | 29.167 | 73.098 | 20.490 | 1.00 | 58.35 |
| 1228 | CD | GLN | A | 494 | 28.934 | 72.671 | 19.058 | 1.00 | 65.11 |
| 1229 | OE1 | GLN | A | 494 | 28.391 | 71.597 | 18.797 | 1.00 | 71.67 |
| 1230 | NE2 | GLN | A | 494 | 29.345 | 73.512 | 18.118 | 1.00 | 71.87 |
| 1231 | N | PRO | A | 495 | 24.661 | 72.598 | 21.272 | 1.00 | 36.89 |
| 1232 | CA | PRO | A | 495 | 23.470 | 72.278 | 22.063 | 1.00 | 38.37 |
| 1233 | C | PRO | A | 495 | 23.863 | 71.991 | 23.513 | 1.00 | 40.80 |
| 1234 | O | PRO | A | 495 | 24.837 | 71.286 | 23.773 | 1.00 | 40.76 |
| 1235 | CB | PRO | A | 495 | 22.920 | 71.043 | 21.371 | 1.00 | 37.83 |
| 1236 | CD | PRO | A | 495 | 23.300 | 71.285 | 19.926 | 1.00 | 40.41 |
| 1237 | CD | PRO | A | 495 | 24.716 | 71.791 | 20.039 | 1.00 | 35.81 |
| 1238 | N | ARG | A | 496 | 23.120 | 72.560 | 24.450 | 1.00 | 42.05 |
| 1239 | CA | ARG | A | 496 | 23.392 | 72.345 | 25.860 | 1.00 | 47.69 |
| 1240 | C | ARG | A | 496 | 22.146 | 71.828 | 26.558 | 1.00 | 51.66 |
| 1241 | O | ARG | A | 496 | 21.042 | 72.317 | 26.316 | 1.00 | 51.95 |
| 1242 | CB | ARG | A | 496 | 23.849 | 73.646 | 26.516 | 1.00 | 45.81 |
| 1243 | CG | ARG | A | 496 | 25.283 | 74.001 | 26.208 | 1.00 | 51.53 |
| 1244 | CD | ARG | A | 496 | 25.653 | 75.383 | 26.709 | 1.00 | 53.72 |
| 1245 | NE | ARG | A | 496 | 27.046 | 75.425 | 27.137 | 1.00 | 57.50 |
| 1246 | CZ | ARG | A | 496 | 27.478 | 74.992 | 28.317 | 1.00 | 58.59 |
| 1247 | NH1 | ARG | A | 496 | 26.626 | 74.489 | 29.199 | 1.00 | 57.22 |
| 1248 | NE2 | ARG | A | 496 | 28.768 | 75.058 | 28.612 | 1.00 | 65.31 |
| 1249 | N | LYS | A | 497 | 22.319 | 70.827 | 27.413 | 1.00 | 56.98 |
| 1250 | CA | LYS | A | 497 | 21.192 | 70.265 | 28.147 | 1.00 | 61.91 |
| 1251 | C | LYS | A | 497 | 20.733 | 71.299 | 29.145 | 1.00 | 63.97 |
| 1252 | O | LYS | A | 497 | 21.405 | 72.300 | 29.371 | 1.00 | 64.61 |
| 1253 | CD | LYS | A | 497 | 21.597 | 69.013 | 28.928 | 1.00 | 66.35 |
| 1254 | CG | LYS | A | 497 | 21.819 | 67.752 | 28.113 | 1.00 | 74.14 |
| 1255 | CD | LYS | A | 497 | 22.241 | 66.607 | 29.034 | 1.00 | 80.69 |
| 1256 | CE | LYS | A | 497 | 22.408 | 65.296 | 28.286 | 1.00 | 84.48 |
| 1257 | NZ | LYS | A | 497 | 22.861 | 64.215 | 29.211 | 1.00 | 88.26 |
| 1258 | N | THR | A | 498 | 19.583 | 71.045 | 29.748 | 1.00 | 67.67 |
| 1259 | CA | THR | A | 498 | 19.028 | 71.931 | 30.758 | 1.00 | 71.26 |
| 1260 | C | THR | A | 498 | 18.316 | 71.050 | 31.760 | 1.00 | 73.04 |
| 1261 | O | THR | A | 498 | 18.610 | 69.857 | 31.864 | 1.00 | 72.83 |
| 1262 | CB | THR | A | 498 | 18.015 | 72.921 | 30.155 | 1.00 | 71.80 |
| 1263 | OG1 | THR | A | 498 | 17.045 | 72.208 | 29.379 | 1.00 | 71.58 |
| 1264 | CG2 | THR | A | 498 | 18.722 | 73.930 | 29.281 | 1.00 | 73.61 |
| 1265 | N | LYS | A | 499 | 17.380 | 71.638 | 32.495 | 1.00 | 75.57 |
| 1266 | CA | LYS | A | 499 | 16.607 | 70.895 | 33.480 | 1.00 | 77.62 |
| 1267 | C | LYS | A | 499 | 15.458 | 70.173 | 32.779 | 1.00 | 77.92 |
| 1268 | O | LYS | A | 499 | 14.504 | 70.809 | 32.332 | 1.00 | 79.19 |
| 1269 | CD | LYS | A | 499 | 16.042 | 71.848 | 34.533 | 1.00 | 80.22 |
| 1270 | CG | LYS | A | 499 | 17.069 | 72.428 | 35.496 | 1.00 | 81.04 |
| 1271 | CD | LYS | A | 499 | 17.507 | 71.397 | 36.529 | 1.00 | 83.26 |
| 1272 | CE | LYS | A | 499 | 18.368 | 72.031 | 37.613 | 1.00 | 84.81 |
| 1273 | NZ | LYS | A | 499 | 18.736 | 71.055 | 38.675 | 1.00 | 86.04 |
| 1274 | N | GLY | A | 500 | 15.566 | 68.852 | 32.667 | 1.00 | 76.91 |
| 1275 | CA | GLY | A | 500 | 14.523 | 68.056 | 32.039 | 1.00 | 75.83 |
| 1276 | C | GLY | A | 500 | 14.040 | 68.459 | 30.655 | 1.00 | 75.14 |
| 1277 | O | GLY | A | 500 | 14.024 | 67.629 | 29.745 | 1.00 | 75.52 |
| 1278 | N | SER | A | 501 | 13.642 | 69.718 | 30.495 | 1.00 | 73.08 |
| 1279 | CA | SER | A | 501 | 13.132 | 70.230 | 29.225 | 1.00 | 71.65 |
| 1280 | C | SER | A | 501 | 13.764 | 69.592 | 27.994 | 1.00 | 68.64 |
| 1281 | O | SER | A | 501 | 13.085 | 68.910 | 27.223 | 1.00 | 71.13 |
| 1282 | CB | SER | A | 501 | 13.321 | 71.746 | 29.147 | 1.00 | 74.43 |
| 1283 | OG | SER | A | 501 | 14.667 | 72.076 | 28.859 | 1.00 | 80.92 |
| 1284 | N | GLY | A | 502 | 15.060 | 69.814 | 27.810 | 1.00 | 63.14 |
| 1285 | CA | GLY | A | 502 | 15.741 | 69.256 | 26.657 | 1.00 | 54.62 |
| 1286 | C | GLY | A | 502 | 16.982 | 70.048 | 26.297 | 1.00 | 48.04 |
| 1287 | O | GLY | A | 502 | 17.917 | 70.131 | 27.094 | 1.00 | 48.08 |
| 1288 | N | PHE | A | 503 | 17.001 | 70.642 | 25.106 | 1.00 | 40.50 |
| 1289 | CA | PHE | A | 503 | 18.173 | 71.411 | 24.692 | 1.00 | 33.81 |
| 1290 | C | PHE | A | 503 | 17.908 | 72.827 | 24.212 | 1.00 | 28.39 |
| 1291 | O | PHE | A | 503 | 16.789 | 73.192 | 23.856 | 1.00 | 26.35 |
| 1292 | CB | PHE | A | 503 | 18.938 | 70.681 | 23.583 | 1.00 | 35.09 |
| 1293 | CC | PHE | A | 503 | 19.452 | 69.337 | 23.981 | 1.00 | 34.16 |
| 1294 | CD1 | PHE | A | 503 | 18.630 | 68.219 | 23.926 | 1.00 | 35.05 |
| 1295 | CD2 | PHE | A | 503 | 20.755 | 69.189 | 24.430 | 1.00 | 35.90 |
| 1296 | CE1 | PHE | A | 503 | 19.099 | 66.967 | 24.317 | 1.00 | 36.00 |
| 1297 | CE2 | PHE | A | 503 | 21.238 | 67.944 | 24.826 | 1.00 | 40.04 |
| 1298 | CZ | PHE | A | 503 | 20.408 | 66.829 | 24.771 | 1.00 | 38.82 |
| 1299 | N | PHE | A | 504 | 18.967 | 73.625 | 24.216 | 1.00 | 26.16 |
| 1300 | CA | PHE | A | 504 | 18.891 | 74.988 | 23.731 | 1.00 | 25.00 |
| 1301 | C | PHE | A | 504 | 20.182 | 75.314 | 23.004 | 1.00 | 26.57 |
| 1302 | O | PHE | A | 504 | 21.221 | 74.708 | 23.267 | 1.00 | 26.73 |
| 1303 | CB | PHE | A | 504 | 18.640 | 75.984 | 24.858 | 1.00 | 22.46 |
| 1304 | CG | PHE | A | 504 | 19.834 | 76.270 | 25.718 | 1.00 | 27.70 |
| 1305 | CU | PHE | A | 504 | 20.674 | 77.340 | 25.433 | 1.00 | 28.94 |
| 1306 | CD2 | PHE | A | 504 | 20.083 | 75.514 | 26.857 | 1.00 | 31.34 |
| 1307 | CE1 | PHE | A | 504 | 21.743 | 77.662 | 26.273 | 1.00 | 30.08 |
| 1308 | CE2 | PHE | A | 504 | 21.151 | 75.828 | 27.704 | 1.00 | 32.65 |
| 1309 | CZ | PHE | A | 504 | 21.979 | 76.909 | 27.403 | 1.00 | 32.80 |
| 1310 | N | VAL | A | 505 | 20.091 | 76.270 | 22.085 | 1.00 | 23.68 |
| 1311 | CA | VAL | A | 505 | 21.211 | 76.705 | 21.286 | 1.00 | 24.67 |
| 1312 | C | VAL | A | 505 | 21.041 | 78.215 | 21.072 | 1.00 | 23.86 |
| 1313 | O | VAL | A | 505 | 19.928 | 78.725 | 21.049 | 1.00 | 21.24 |
| 1314 | CB | VAL | A | 505 | 21.208 | 75.926 | 19.939 | 1.00 | 27.95 |
| 1315 | CG1 | VAL | A | 505 | 20.134 | 76.464 | 19.020 | 1.00 | 25.61 |
| 1316 | CG2 | VAL | A | 505 | 22.558 | 75.957 | 19.314 | 1.00 | 33.51 |
| 1317 | N | PHE | A | 506 | 22.146 | 78.936 | 20.959 | 1.00 | 26.20 |
| 1318 | CA | PHE | A | 506 | 22.086 | 80.382 | 20.754 | 1.00 | 26.10 |
| 1319 | C | PHE | A | 506 | 22.846 | 80.745 | 19.479 | 1.00 | 24.83 |
| 1320 | O | PHE | A | 506 | 23.928 | 80.231 | 19.241 | 1.00 | 25.52 |
| 1321 | CB | PHE | A | 506 | 22.725 | 81.104 | 21.939 | 1.00 | 28.18 |
| 1322 | CG | PHE | A | 506 | 22.758 | 82.593 | 21.797 | 1.00 | 35.33 |
| 1323 | CD1 | PHE | A | 506 | 21.620 | 83.355 | 22.020 | 1.00 | 42.56 |
| 1324 | CD2 | PHE | A | 506 | 23.937 | 83.241 | 21.443 | 1.00 | 40.04 |
| 1325 | CE1 | PHE | A | 506 | 21.660 | 84.746 | 21.892 | 1.00 | 44.70 |
| 1326 | CR2 | PHE | A | 506 | 23.987 | 84.623 | 21.311 | 1.00 | 40.54 |
| 1327 | CZ | PHE | A | 506 | 22.851 | 85.377 | 21.536 | 1.00 | 42.12 |
| 1328 | N | SER | A | 507 | 22.290 | 81.639 | 18.673 | 1.00 | 24.69 |
| 1329 | CA | SER | A | 507 | 22.941 | 82.050 | 17.426 | 1.00 | 24.73 |
| 1330 | C | SER | A | 507 | 23.065 | 83.578 | 17.355 | 1.00 | 26.40 |
| 1331 | O | SER | A | 507 | 22.113 | 84.292 | 17.609 | 1.00 | 25.99 |
| 1332 | CB | SER | A | 507 | 22.151 | 81.521 | 16.221 | 1.00 | 22.19 |
| 1333 | OG | SER | A | 507 | 22.740 | 81.947 | 15.009 | 1.00 | 30.30 |
| 1334 | N | ARG | A | 508 | 24.255 | 84.062 | 17.009 | 1.00 | 26.98 |
| 1335 | CA | ARG | A | 508 | 24.545 | 85.496 | 16.913 | 1.00 | 25.95 |
| 1336 | C | ARG | A | 508 | 24.851 | 85.911 | 15.463 | 1.00 | 24.97 |
| 1337 | O | ARG | A | 508 | 25.621 | 85.250 | 14.785 | 1.00 | 24.35 |
| 1338 | CB | ARG | A | 508 | 25.755 | 85.801 | 17.790 | 1.00 | 21.97 |
| 1339 | CC | ARG | A | 508 | 26.249 | 87.235 | 17.756 | 1.00 | 25.32 |
| 1340 | CD | ARG | A | 508 | 27.474 | 87.372 | 18.656 | 1.00 | 25.67 |
| 1341 | NE | ARG | A | 508 | 27.898 | 88.758 | 18.788 | 1.00 | 36.00 |
| 1342 | CZ | ARG | A | 508 | 28.757 | 89.357 | 17.972 | 1.00 | 36.06 |
| 1343 | NH1 | ARG | A | 508 | 29.293 | 88.682 | 16.963 | 1.00 | 34.19 |
| 1344 | NH2 | ARG | A | 508 | 29.062 | 90.633 | 18.157 | 1.00 | 34.70 |
| 1345 | N | LEU | A | 509 | 24.269 | 87.017 | 15.001 | 1.00 | 26.44 |
| 1346 | CA | LEU | A | 509 | 24.492 | 87.479 | 13.621 | 1.00 | 28.45 |
| 1347 | C | LEU | A | 509 | 24.794 | 88.973 | 13.540 | 1.00 | 28.87 |
| 1348 | O | LEU | A | 509 | 23.893 | 89.797 | 13.699 | 1.00 | 30.99 |
| 1349 | CB | LEU | A | 509 | 23.259 | 87.177 | 12.758 | 1.00 | 27.28 |
| 1350 | CG | LEU | A | 509 | 23.270 | 87.733 | 11.321 | 1.00 | 31.90 |
| 1351 | CD1 | LEU | A | 509 | 24.284 | 86.985 | 10.453 | 1.00 | 29.48 |
| 1352 | CD2 | LEU | A | 509 | 21.886 | 87.599 | 10.732 | 1.00 | 28.89 |
| 1353 | N | GLU | A | 510 | 26.054 | 89.323 | 13.288 | 1.00 | 31.92 |
| 1354 | CA | GLU | A | 510 | 26.448 | 90.728 | 13.189 | 1.00 | 34.67 |
| 1355 | C | GLU | A | 510 | 26.005 | 91.290 | 11.845 | 1.00 | 36.35 |
| 1356 | O | GLU | A | 510 | 26.199 | 90.652 | 10.812 | 1.00 | 34.66 |
| 1357 | CB | GLU | A | 510 | 27.970 | 90.875 | 13.343 | 1.00 | 41.07 |
| 1358 | CG | GLU | A | 510 | 28.510 | 90.383 | 14.696 | 1.00 | 54.35 |
| 1359 | CD | GLU | A | 510 | 29.997 | 90.675 | 14.911 | 1.00 | 58.86 |
| 1360 | OE1 | GLU | A | 510 | 30.811 | 90.364 | 14.013 | 1.00 | 61.22 |
| 1361 | OE2 | GLU | A | 510 | 30.349 | 91.207 | 15.989 | 1.00 | 61.34 |
| 1362 | N | VAL | A | 511 | 25.406 | 92.479 | 11.852 | 1.00 | 36.50 |
| 1363 | CA | VAL | A | 511 | 24.935 | 93.069 | 10.606 | 1.00 | 38.05 |
| 1364 | C | VAL | A | 511 | 25.370 | 94.516 | 10.408 | 1.00 | 42.41 |
| 1365 | O | VAL | A | 511 | 25.744 | 95.205 | 11.368 | 1.00 | 41.92 |
| 1366 | CB | VAL | A | 511 | 23.403 | 93.012 | 10.508 | 1.00 | 31.84 |
| 1367 | CD1 | VAL | A | 511 | 22.927 | 91.585 | 10.669 | 1.00 | 32.42 |
| 1368 | CG2 | VAL | A | 511 | 22.787 | 93.913 | 11.553 | 1.00 | 26.87 |
| 1369 | N | THR | A | 512 | 25.310 | 94.967 | 9.154 | 1.00 | 43.58 |
| 1370 | CA | THR | A | 512 | 25.697 | 96.331 | 8.808 | 1.00 | 48.37 |
| 1371 | C | THR | A | 512 | 24.602 | 97.306 | 9.201 | 1.00 | 50.53 |
| 1372 | O | THR | A | 512 | 23.487 | 96.894 | 9.516 | 1.00 | 51.77 |
| 1373 | CB | THR | A | 512 | 25.956 | 96.475 | 7.303 | 1.00 | 48.57 |
| 1374 | OG1 | THR | A | 512 | 24.766 | 96.140 | 6.581 | 1.00 | 46.96 |
| 1375 | CG2 | THR | A | 512 | 27.085 | 95.554 | 6.866 | 1.00 | 45.82 |
| 1376 | N | ARG | A | 513 | 24.922 | 98.598 | 9.188 | 1.00 | 53.29 |
| 1377 | CA | ARG | A | 513 | 23.952 | 99.629 | 9.544 | 1.00 | 55.54 |
| 1378 | C | ARG | A | 513 | 22.781 | 99.557 | 8.577 | 1.00 | 55.38 |
| 1379 | O | ARG | A | 513 | 21.623 | 99.741 | 8.962 | 1.00 | 55.22 |
| 1380 | CB | ARG | A | 513 | 24.594 | 101.018 | 9.464 | 1.00 | 59.23 |
| 1381 | CG | ARG | A | 513 | 23.999 | 102.037 | 10.428 | 1.00 | 65.98 |
| 1382 | CD | ARG | A | 513 | 22.519 | 102.274 | 10.184 | 1.00 | 74.12 |
| 1383 | NE | ARG | A | 513 | 21.858 | 102.827 | 11.365 | 1.00 | 80.97 |
| 1384 | CZ | ARG | A | 513 | 20.581 | 103.197 | 11.413 | 1.00 | 83.64 |
| 1385 | NH1 | ARG | A | 513 | 19.809 | 103.084 | 10.340 | 1.00 | 86.02 |
| 1386 | NN2 | ARG | A | 513 | 20.072 | 103.673 | 12.543 | 1.00 | 85.44 |
| 1387 | N | ALA | A | 514 | 23.099 | 99.285 | 7.317 | 1.00 | 55.03 |
| 1388 | CA | ALA | A | 514 | 22.096 | 99.186 | 6.268 | 1.00 | 55.46 |
| 1389 | C | ALA | A | 514 | 21.061 | 98.104 | 6.571 | 1.00 | 56.10 |
| 1390 | O | ALA | A | 514 | 19.866 | 98.390 | 6.674 | 1.00 | 56.61 |
| 1391 | CB | ALA | A | 514 | 22.775 | 98.908 | 4.927 | 1.00 | 54.05 |
| 1392 | N | GLU | A | 515 | 21.509 | 96.859 | 6.712 | 1.00 | 55.46 |
| 1393 | CA | GLU | A | 515 | 20.575 | 95.781 | 6.995 | 1.00 | 55.34 |
| 1394 | C | GLU | A | 515 | 19.949 | 95.951 | 8.371 | 1.00 | 54.83 |
| 1395 | O | GLU | A | 515 | 18.877 | 95.410 | 8.644 | 1.00 | 52.33 |
| 1396 | CB | GLU | A | 515 | 21.265 | 94.413 | 6.868 | 1.00 | 57.76 |
| 1397 | CG | GLU | A | 515 | 22.716 | 94.379 | 7.312 | 1.00 | 62.58 |
| 1398 | CD | GLU | A | 515 | 23.416 | 93.082 | 6.937 | 1.00 | 63.27 |
| 1399 | OE1 | GLU | A | 515 | 23.168 | 92.565 | 5.829 | 1.00 | 64.89 |
| 1400 | OE2 | GLU | A | 515 | 24.228 | 92.586 | 7.741 | 1.00 | 66.59 |
| 1401 | N | TRP | A | 516 | 20.608 | 96.716 | 9.234 | 1.00 | 55.79 |
| 1402 | CA | TRP | A | 516 | 20.072 | 96.956 | 10.566 | 1.00 | 57.81 |
| 1403 | C | TRP | A | 516 | 18.818 | 97.822 | 10.464 | 1.00 | 58.51 |
| 1404 | O | TRP | A | 516 | 17.988 | 97.822 | 11.374 | 1.00 | 57.71 |
| 1405 | CB | TRP | A | 516 | 21.101 | 97.649 | 11.466 | 1.00 | 61.48 |
| 1406 | CG | TRP | A | 516 | 20.738 | 97.544 | 12.913 | 1.00 | 66.54 |
| 1407 | CD1 | TRP | A | 516 | 20.823 | 96.428 | 13.697 | 1.00 | 67.26 |
| 1408 | CD2 | TRP | A | 516 | 20.140 | 98.561 | 13.725 | 1.00 | 69.56 |
| 1409 | NE1 | TRP | A | 516 | 20.309 | 96.685 | 14.946 | 1.00 | 71.76 |
| 1410 | CE2 | TRP | A | 516 | 19.882 | 97.987 | 14.990 | 1.00 | 70.88 |
| 1411 | CE3 | TRP | A | 516 | 19.793 | 99.900 | 13.507 | 1.00 | 70.94 |
| 1412 | CZ2 | TRP | A | 516 | 19.292 | 98.706 | 16.034 | 1.00 | 73.50 |
| 1413 | CZ3 | TRP | A | 516 | 19.204 | 100.618 | 14.548 | 1.00 | 74.37 |
| 1414 | CH2 | TRP | A | 516 | 18.961 | 100.016 | 15.796 | 1.00 | 74.71 |
| 1415 | N | GLU | A | 517 | 18.682 | 98.553 | 9.356 | 1.00 | 59.66 |
| 1416 | CA | GLU | A | 517 | 17.515 | 99.409 | 9.128 | 1.00 | 61.72 |
| 1417 | C | GLU | A | 517 | 16.301 | 98.565 | 8.746 | 1.00 | 59.87 |
| 1418 | O | GLU | A | 517 | 15.159 | 98.994 | 8.904 | 1.00 | 58.60 |
| 1419 | CB | GLU | A | 517 | 17.782 | 100.418 | 8.003 | 1.00 | 68.82 |
| 1420 | CD | GLU | A | 517 | 18.988 | 101.316 | 8.221 | 1.00 | 80.60 |
| 1421 | CD | GLU | A | 517 | 19.148 | 102.358 | 7.124 | 1.00 | 87.42 |
| 1422 | GE1 | GLU | A | 517 | 18.472 | 102.231 | 6.079 | 1.00 | 94.80 |
| 1423 | OE2 | GLU | A | 517 | 19.955 | 103.299 | 7.300 | 1.00 | 95.35 |
| 1424 | N | GLN | A | 518 | 16.555 | 97.366 | 8.233 | 1.00 | 57.92 |
| 1425 | CA | GLN | A | 518 | 15.480 | 96.468 | 7.836 | 1.00 | 55.74 |
| 1426 | C | GLN | A | 518 | 15.543 | 95.184 | 8.664 | 1.00 | 54.32 |
| 1427 | O | GLN | A | 518 | 15.207 | 94.102 | 8.176 | 1.00 | 52.85 |
| 1428 | CB | GLN | A | 518 | 15.600 | 96.146 | 6.355 | 1.00 | 54.10 |
| 1429 | N | LYS | A | 519 | 15.954 | 95.313 | 9.924 | 1.00 | 52.69 |
| 1430 | CA | LYS | A | 519 | 16.082 | 94.152 | 10.791 | 1.00 | 52.35 |
| 1431 | C | LYS | A | 519 | 14.781 | 93.394 | 10.941 | 1.00 | 51.69 |
| 1432 | O | LYS | A | 519 | 14.778 | 92.203 | 11.246 | 1.00 | 49.61 |
| 1433 | CB | LYS | A | 519 | 16.617 | 94.561 | 12.163 | 1.00 | 54.31 |
| 1434 | CG | LYS | A | 519 | 15.688 | 95.395 | 13.012 | 1.00 | 55.80 |
| 1435 | CD | LYS | A | 519 | 16.438 | 95.877 | 14.241 | 1.00 | 60.32 |
| 1436 | CE | LYS | A | 519 | 15.598 | 96.806 | 15.089 | 1.00 | 64.05 |
| 1437 | NZ | LYS | A | 519 | 16.409 | 97.377 | 16.202 | 1.00 | 70.92 |
| 1438 | N | ASP | A | 520 | 13.676 | 94.089 | 10.712 | 1.00 | 52.66 |
| 1439 | CA | ASP | A | 520 | 12.349 | 93.495 | 10.812 | 1.00 | 54.22 |
| 1440 | C | ASP | A | 520 | 12.204 | 92.391 | 9.764 | 1.00 | 53.07 |
| 1441 | O | ASP | A | 520 | 11.336 | 91.525 | 9.871 | 1.00 | 52.75 |
| 1442 | CB | ASP | A | 520 | 11.289 | 94.568 | 10.563 | 1.00 | 61.14 |
| 1443 | CD | ASP | A | 520 | 11.821 | 95.980 | 10.792 | 1.00 | 71.19 |
| 1444 | OD1 | ASP | A | 520 | 12.193 | 96.300 | 11.945 | 1.00 | 76.53 |
| 1445 | OD2 | ASP | A | 520 | 11.871 | 96.766 | 9.817 | 1.00 | 72.86 |
| 1446 | N | GLU | A | 521 | 13.067 | 92.423 | 8.757 | 1.00 | 51.01 |
| 1447 | CA | GLU | A | 521 | 13.018 | 91.443 | 7.684 | 1.00 | 50.49 |
| 1448 | C | GLU | A | 521 | 13.886 | 90.206 | 7.899 | 1.00 | 47.73 |
| 1449 | O | GLU | A | 521 | 13.762 | 89.228 | 7.165 | 1.00 | 48.58 |
| 1450 | CB | GLU | A | 521 | 13.400 | 92.107 | 6.362 | 1.00 | 53.54 |
| 1451 | CG | GLU | A | 521 | 12.423 | 93.173 | 5.901 | 1.00 | 60.11 |
| 1452 | CD | GLU | A | 521 | 12.901 | 93.895 | 4.658 | 1.00 | 65.41 |
| 1453 | GE1 | GLU | A | 521 | 13.103 | 93.239 | 3.614 | 1.00 | 67.98 |
| 1454 | OE2 | GLU | A | 521 | 13.079 | 95.129 | 4.726 | 1.00 | 73.58 |
| 1455 | N | PHE | A | 522 | 14.772 | 90.241 | 8.887 | 1.00 | 42.66 |
| 1456 | CA | PHE | A | 522 | 15.615 | 89.081 | 9.137 | 1.00 | 38.90 |
| 1457 | C | PHE | A | 522 | 14.847 | 88.017 | 9.892 | 1.00 | 35.93 |
| 1458 | O | PHE | A | 522 | 14.021 | 88.324 | 10.755 | 1.00 | 38.85 |
| 1459 | CB | PHE | A | 522 | 16.884 | 89.478 | 9.886 | 1.00 | 36.56 |
| 1460 | CG | PHE | A | 522 | 17.890 | 90.181 | 9.019 | 1.00 | 39.04 |
| 1461 | CD1 | PHE | A | 522 | 17.673 | 91.494 | 8.605 | 1.00 | 42.28 |
| 1462 | CD2 | PHE | A | 522 | 19.026 | 89.513 | 8.561 | 1.00 | 36.78 |
| 1463 | CE1 | PHE | A | 522 | 18.571 | 92.132 | 7.738 | 1.00 | 44.30 |
| 1464 | CE2 | PHE | A | 522 | 19.927 | 90.139 | 7.695 | 1.00 | 40.92 |
| 1465 | CZ | PHE | A | 522 | 19.699 | 91.454 | 7.283 | 1.00 | 39.51 |
| 1466 | N | ILE | A | 523 | 15.108 | 86.763 | 9.545 | 1.00 | 31.27 |
| 1467 | CA | ILE | A | 523 | 14.427 | 85.641 | 10.156 | 1.00 | 29.65 |
| 1468 | C | ILE | A | 523 | 15.389 | 84.551 | 10.617 | 1.00 | 29.13 |
| 1469 | O | ILE | A | 523 | 16.364 | 84.240 | 9.936 | 1.00 | 27.36 |
| 1470 | CB | ILE | A | 523 | 13.420 | 85.033 | 9.163 | 1.00 | 34.21 |
| 1471 | CG1 | ILE | A | 523 | 12.349 | 86.072 | 8.818 | 1.00 | 37.38 |
| 1472 | CC2 | ILE | A | 523 | 12.804 | 83.773 | 9.736 | 1.00 | 34.94 |
| 1473 | CD1 | ILE | A | 523 | 11.373 | 85.619 | 7.746 | 1.00 | 38.56 |
| 1474 | N | CYS | A | 524 | 15.102 | 83.990 | 11.789 | 1.00 | 26.34 |
| 1475 | CA | CYS | A | 524 | 15.901 | 82.914 | 12.361 | 1.00 | 27.10 |
| 1476 | C | CYS | A | 524 | 15.100 | 81.654 | 12.138 | 1.00 | 26.67 |
| 1477 | O | CYS | A | 524 | 13.945 | 81.590 | 12.531 | 1.00 | 27.58 |
| 1478 | CB | CYS | A | 524 | 16.103 | 83.127 | 13.874 | 1.00 | 24.27 |
| 1479 | SG | CYS | A | 524 | 16.974 | 81.777 | 14.737 | 1.00 | 37.60 |
| 1480 | N | ARG | A | 525 | 15.714 | 80.649 | 11.528 | 1.00 | 26.37 |
| 1481 | CA | ARG | A | 525 | 15.024 | 79.406 | 11.244 | 1.00 | 26.35 |
| 1482 | C | ARG | A | 525 | 15.713 | 78.181 | 11.796 | 1.00 | 26.72 |
| 1483 | O | ARG | A | 525 | 16.929 | 78.039 | 11.700 | 1.00 | 29.04 |
| 1484 | CB | ARG | A | 525 | 14.852 | 79.224 | 9.739 | 1.00 | 29.81 |
| 1485 | CG | ARG | A | 525 | 14.207 | 77.887 | 9.372 | 1.00 | 35.40 |
| 1486 | CD | ARG | A | 525 | 13.542 | 77.948 | 8.001 | 1.00 | 46.51 |
| 1487 | NE | ARG | A | 525 | 14.487 | 77.804 | 6.907 | 1.00 | 50.77 |
| 1488 | CZ | ARG | A | 525 | 14.221 | 78.139 | 5.649 | 1.00 | 55.09 |
| 1489 | NH1 | ARG | A | 525 | 13.035 | 78.649 | 5.337 | 1.00 | 54.58 |
| 1490 | NH2 | ARG | A | 525 | 15.136 | 77.950 | 4.703 | 1.00 | 56.77 |
| 1491 | N | ALA | A | 526 | 14.920 | 77.289 | 12.369 | 1.00 | 25.38 |
| 1492 | CA | ALA | A | 526 | 15.458 | 76.072 | 12.920 | 1.00 | 25.38 |
| 1493 | C | ALA | A | 526 | 14.872 | 74.879 | 12.175 | 1.00 | 25.99 |
| 1494 | O | ALA | A | 526 | 13.667 | 74.849 | 11.890 | 1.00 | 25.50 |
| 1495 | CB | ALA | A | 526 | 15.116 | 75.971 | 14.404 | 1.00 | 25.98 |
| 1496 | N | VAL | A | 527 | 15.728 | 73.916 | 11.841 | 1.00 | 23.84 |
| 1497 | CA | VAL | A | 527 | 15.267 | 72.705 | 11.198 | 1.00 | 25.15 |
| 1498 | C | VAL | A | 527 | 15.522 | 71.628 | 12.220 | 1.00 | 26.30 |
| 1499 | O | VAL | A | 527 | 16.642 | 71.481 | 12.706 | 1.00 | 26.20 |
| 1500 | CB | VAL | A | 527 | 16.043 | 72.365 | 9.923 | 1.00 | 24.43 |
| 1501 | CG1 | VAL | A | 527 | 15.610 | 71.012 | 9.439 | 1.00 | 26.95 |
| 1502 | CG2 | VAL | A | 527 | 15.781 | 73.409 | 8.849 | 1.00 | 28.13 |
| 1503 | N | HIS | A | 528 | 14.485 | 70.862 | 12.531 | 1.00 | 26.24 |
| 1504 | CA | HIS | A | 528 | 14.582 | 69.828 | 13.536 | 1.00 | 29.14 |
| 1505 | C | HIS | A | 528 | 13.498 | 68.778 | 13.295 | 1.00 | 31.86 |
| 1506 | O | HIS | A | 528 | 12.384 | 69.091 | 12.861 | 1.00 | 31.05 |
| 1507 | CB | HIS | A | 528 | 14.428 | 70.479 | 14.921 | 1.00 | 29.03 |
| 1508 | CC | HIS | A | 528 | 14.518 | 69.523 | 16.065 | 1.00 | 29.34 |
| 1509 | ND1 | HIS | A | 528 | 13.491 | 68.666 | 16.404 | 1.00 | 33.33 |
| 1510 | CD2 | HIS | A | 528 | 15.510 | 69.283 | 16.956 | 1.00 | 28.93 |
| 1511 | CE1 | HIS | A | 528 | 13.845 | 67.945 | 17.449 | 1.00 | 26.07 |
| 1512 | NE2 | HIS | A | 528 | 15.070 | 68.298 | 17.805 | 1.00 | 32.19 |
| 1513 | N | GLU | A | 529 | 13.843 | 67.536 | 13.599 | 1.00 | 34.08 |
| 1514 | CA | GLU | A | 529 | 12.964 | 66.393 | 13.424 | 1.00 | 39.76 |
| 1515 | C | GLU | A | 529 | 11.563 | 66.490 | 14.034 | 1.00 | 41.50 |
| 1516 | O | GLU | A | 529 | 10.580 | 66.190 | 13.360 | 1.00 | 41.48 |
| 1517 | CB | GLU | A | 529 | 13.641 | 65.138 | 13.978 | 1.00 | 43.03 |
| 1518 | CG | GLU | A | 529 | 12.702 | 63.935 | 14.014 | 1.00 | 56.19 |
| 1519 | CD | GLU | A | 529 | 13.239 | 62.773 | 14.826 | 1.00 | 62.66 |
| 1520 | OE1 | GLU | A | 529 | 12.489 | 61.788 | 15.007 | 1.00 | 63.52 |
| 1521 | OE2 | GLU | A | 529 | 14.402 | 62.842 | 15.281 | 1.00 | 65.43 |
| 1522 | N | ALA | A | 530 | 11.479 | 66.897 | 15.300 | 1.00 | 42.51 |
| 1523 | CA | ALA | A | 530 | 10.201 | 66.978 | 16.006 | 1.00 | 46.03 |
| 1524 | C | ALA | A | 530 | 9.269 | 68.105 | 15.596 | 1.00 | 49.29 |
| 1525 | O | ALA | A | 530 | 8.124 | 68.153 | 16.044 | 1.00 | 50.87 |
| 1526 | CB | ALA | A | 530 | 10.443 | 67.043 | 17.513 | 1.00 | 39.71 |
| 1527 | N | ALA | A | 531 | 9.751 | 69.005 | 14.748 | 1.00 | 52.89 |
| 1528 | CA | ALA | A | 531 | 8.949 | 70.133 | 14.302 | 1.00 | 56.14 |
| 1529 | C | ALA | A | 531 | 8.030 | 69.799 | 13.124 | 1.00 | 59.66 |
| 1530 | O | ALA | A | 531 | 8.258 | 70.243 | 12.004 | 1.00 | 61.00 |
| 1531 | CB | ALA | A | 531 | 9.864 | 71.295 | 13.938 | 1.00 | 52.83 |
| 1532 | N | SER | A | 532 | 6.992 | 69.012 | 13.370 | 1.00 | 63.07 |
| 1533 | CA | SER | A | 532 | 6.056 | 68.673 | 12.305 | 1.00 | 66.03 |
| 1534 | C | SER | A | 532 | 5.006 | 69.778 | 12.250 | 1.00 | 66.08 |
| 1535 | O | SER | A | 532 | 4.569 | 70.283 | 13.286 | 1.00 | 65.35 |
| 1536 | CB | SER | A | 532 | 5.384 | 67.330 | 12.593 | 1.00 | 68.98 |
| 1537 | OG | SER | A | 532 | 4.526 | 67.416 | 13.716 | 1.00 | 74.64 |
| 1538 | N | PRO | A | 533 | 4.581 | 70.173 | 11.043 | 1.00 | 66.51 |
| 1539 | CA | PRO | A | 533 | 4.931 | 69.713 | 9.696 | 1.00 | 65.99 |
| 1540 | C | PRO | A | 533 | 6.251 | 70.226 | 9.117 | 1.00 | 63.48 |
| 1541 | O | PRO | A | 533 | 6.760 | 71.273 | 9.518 | 1.00 | 64.33 |
| 1542 | CD | PRO | A | 533 | 3.757 | 70.203 | 8.873 | 1.00 | 69.06 |
| 1543 | CG | PRO | A | 533 | 3.539 | 71.556 | 9.471 | 1.00 | 71.08 |
| 1544 | CD | PRO | A | 533 | 3.570 | 71.242 | 10.963 | 1.00 | 70.23 |
| 1545 | N | SER | A | 534 | 6.783 | 69.466 | 8.165 | 1.00 | 59.21 |
| 1546 | CA | SER | A | 534 | 8.009 | 69.804 | 7.452 | 1.00 | 55.01 |
| 1547 | C | SER | A | 534 | 9.274 | 70.148 | 8.248 | 1.00 | 50.27 |
| 1548 | O | SER | A | 534 | 10.130 | 70.875 | 7.753 | 1.00 | 48.58 |
| 1549 | CD | SER | A | 534 | 7.706 | 70.934 | 6.475 | 1.00 | 58.20 |
| 1550 | OG | SER | A | 534 | 7.074 | 72.013 | 7.138 | 1.00 | 63.36 |
| 1551 | N | GLN | A | 535 | 9.387 | 69.616 | 9.461 | 1.00 | 44.89 |
| 1552 | CA | GLN | A | 535 | 10.543 | 69.829 | 10.330 | 1.00 | 42.88 |
| 1553 | C | GLN | A | 535 | 11.194 | 71.205 | 10.277 | 1.00 | 41.55 |
| 1554 | O | GLN | A | 535 | 12.419 | 71.321 | 10.294 | 1.00 | 40.80 |
| 1555 | CB | GLN | A | 535 | 11.633 | 68.769 | 10.080 | 1.00 | 37.08 |
| 1556 | CG | GLN | A | 535 | 11.302 | 67.731 | 9.056 | 1.00 | 47.40 |
| 1557 | CD | GLN | A | 535 | 10.101 | 66.916 | 9.431 | 1.00 | 44.36 |
| 1558 | OE1 | GLN | A | 535 | 9.193 | 66.738 | 8.625 | 1.00 | 47.59 |
| 1559 | NE2 | GLN | A | 535 | 10.079 | 66.415 | 10.664 | 1.00 | 54.70 |
| 1560 | N | THR | A | 536 | 10.387 | 72.252 | 10.226 | 1.00 | 39.97 |
| 1561 | CA | THR | A | 536 | 10.950 | 73.588 | 10.207 | 1.00 | 40.62 |
| 1562 | C | THR | A | 536 | 10.113 | 74.575 | 11.023 | 1.00 | 37.57 |
| 1563 | O | THR | A | 536 | 8.893 | 74.558 | 10.975 | 1.00 | 40.85 |
| 1564 | CB | THR | A | 536 | 11.137 | 74.092 | 8.747 | 1.00 | 42.38 |
| 1565 | OG1 | THR | A | 536 | 11.458 | 75.488 | 8.763 | 1.00 | 44.59 |
| 1566 | CG2 | THR | A | 536 | 9.881 | 73.870 | 7.932 | 1.00 | 49.65 |
| 1567 | N | VAL | A | 537 | 10.788 | 75.410 | 11.801 | 1.00 | 35.29 |
| 1568 | CA | VAL | A | 537 | 10.147 | 76.418 | 12.632 | 1.00 | 31.25 |
| 1569 | C | VAL | A | 537 | 10.996 | 77.678 | 12.537 | 1.00 | 29.01 |
| 1570 | O | VAL | A | 537 | 12.218 | 77.615 | 12.699 | 1.00 | 30.11 |
| 1571 | CB | VAL | A | 537 | 10.106 | 75.996 | 14.117 | 1.00 | 34.80 |
| 1572 | CG1 | VAL | A | 537 | 9.452 | 77.084 | 14.939 | 1.00 | 35.52 |
| 1573 | CG2 | VAL | A | 537 | 9.341 | 74.712 | 14.278 | 1.00 | 37.19 |
| 1574 | N | GLN | A | 538 | 10.365 | 78.820 | 12.292 | 1.00 | 26.73 |
| 1575 | CA | GLN | A | 538 | 11.107 | 80.067 | 12.167 | 1.00 | 26.74 |
| 1576 | C | GLN | A | 538 | 10.376 | 81.271 | 12.732 | 1.00 | 27.53 |
| 1577 | O | CLN | A | 538 | 9.169 | 81.249 | 12.902 | 1.00 | 29.89 |
| 1578 | CB | GLN | A | 538 | 11.424 | 80.335 | 10.700 | 1.00 | 28.48 |
| 1579 | CG | GLN | A | 538 | 10.208 | 80.624 | 9.834 | 1.00 | 25.88 |
| 1580 | CD | GLN | A | 538 | 10.595 | 80.853 | 8.378 | 1.00 | 28.51 |
| 1581 | OE1 | CLN | A | 538 | 11.390 | 80.102 | 7.810 | 1.00 | 31.66 |
| 1582 | NE2 | GLN | A | 538 | 10.035 | 81.885 | 7.772 | 1.00 | 27.39 |
| 1583 | N | ARG | A | 539 | 11.113 | 82.334 | 13.013 | 1.00 | 28.05 |
| 1584 | CA | ARG | A | 539 | 10.493 | 83.530 | 13.535 | 1.00 | 30.72 |
| 1585 | C | ARG | A | 539 | 11.288 | 84.742 | 13.114 | 1.00 | 31.33 |
| 1586 | O | ARG | A | 539 | 12.517 | 84.703 | 13.077 | 1.00 | 32.42 |
| 1587 | CB | ARG | A | 539 | 10.420 | 83.463 | 15.061 | 1.00 | 36.30 |
| 1588 | CG | ARG | A | 539 | 9.488 | 84.476 | 15.686 | 1.00 | 45.60 |
| 1589 | CD | ARG | A | 539 | 8.741 | 83.829 | 16.830 | 1.00 | 57.97 |
| 1590 | NE | ARG | A | 539 | 8.118 | 82.591 | 16.371 | 1.00 | 69.46 |
| 1591 | CZ | ARG | A | 539 | 7.453 | 81.745 | 17.150 | 1.00 | 78.18 |
| 1592 | NH1 | ARG | A | 539 | 7.316 | 81.996 | 18.447 | 1.00 | 83.01 |
| 1593 | NH2 | ARG | A | 539 | 6.927 | 80.641 | 16.630 | 1.00 | 81.89 |
| 1594 | N | ALA | A | 540 | 10.578 | 85.818 | 12.799 | 1.00 | 31.92 |
| 1595 | CA | ALA | A | 540 | 11.207 | 87.054 | 12.383 | 1.00 | 34.50 |
| 1596 | C | ALA | A | 540 | 11.691 | 87.774 | 13.634 | 1.00 | 36.68 |
| 1597 | O | ALA | A | 540 | 11.190 | 87.532 | 14.739 | 1.00 | 34.62 |
| 1598 | CB | ALA | A | 540 | 10.209 | 87.907 | 11.621 | 1.00 | 33.91 |
| 1599 | N | VAL | A | 541 | 12.694 | 88.626 | 13.460 | 1.00 | 39.73 |
| 1600 | CA | VAL | A | 541 | 13.254 | 89.386 | 14.572 | 1.00 | 44.47 |
| 1601 | C | VAL | A | 541 | 12.164 | 90.249 | 15.218 | 1.00 | 46.91 |
| 1602 | O | VAL | A | 541 | 11.595 | 91.124 | 14.573 | 1.00 | 44.53 |
| 1603 | CB | VAL | A | 541 | 14.415 | 90.295 | 14.090 | 1.00 | 42.49 |
| 1604 | CC1 | VAL | A | 541 | 14.715 | 91.358 | 15.130 | 1.00 | 47.96 |
| 1605 | CC2 | VAL | A | 541 | 15.655 | 89.460 | 13.831 | 1.00 | 42.43 |
| 1606 | N | SER | A | 542 | 11.869 | 89.997 | 16.489 | 1.00 | 52.25 |
| 1607 | CA | SER | A | 542 | 10.840 | 90.771 | 17.174 | 1.00 | 60.05 |
| 1608 | C | SER | A | 542 | 11.374 | 92.174 | 17.421 | 1.00 | 64.25 |
| 1609 | O | SER | A | 542 | 12.550 | 92.353 | 17.743 | 1.00 | 65.22 |
| 1610 | CB | SER | A | 542 | 10.443 | 90.110 | 18.500 | 1.00 | 60.43 |
| 1611 | DC | SER | A | 542 | 11.530 | 90.076 | 19.404 | 1.00 | 65.30 |
| 1612 | N | VAL | A | 543 | 10.510 | 93.168 | 17.256 | 1.00 | 68.19 |
| 1613 | CA | VAL | A | 543 | 10.906 | 94.557 | 17.444 | 1.00 | 72.45 |
| 1614 | C | VAL | A | 543 | 9.828 | 95.323 | 18.202 | 1.00 | 74.32 |
| 1615 | O | VAL | A | 543 | 8.970 | 95.979 | 17.603 | 1.00 | 75.99 |
| 1616 | CB | VAL | A | 543 | 11.148 | 95.247 | 16.084 | 1.00 | 74.41 |
| 1617 | CC1 | VAL | A | 543 | 11.530 | 96.705 | 16.299 | 1.00 | 76.37 |
| 1618 | CC2 | VAL | A | 543 | 12.233 | 94.514 | 15.318 | 1.00 | 75.78 |
| 1619 | N | VAL | B | 336 | 42.982 | 59.443 | 29.109 | 1.00 | 58.93 |
| 1620 | CA | VAL | B | 336 | 43.788 | 60.200 | 30.107 | 1.00 | 60.44 |
| 1621 | C | VAL | B | 336 | 44.173 | 61.587 | 29.599 | 1.00 | 60.07 |
| 1622 | O | VAL | B | 336 | 45.211 | 61.753 | 28.958 | 1.00 | 61.48 |
| 1623 | CB | VAL | B | 336 | 45.079 | 59.445 | 30.453 | 1.00 | 60.81 |
| 1624 | CC1 | VAL | B | 336 | 45.794 | 60.136 | 31.600 | 1.00 | 61.71 |
| 1625 | CC2 | VAL | B | 336 | 44.755 | 58.010 | 30.798 | 1.00 | 64.73 |
| 1626 | N | SER | B | 337 | 43.343 | 62.583 | 29.891 | 1.00 | 57.95 |
| 1627 | CA | SER | B | 337 | 43.617 | 63.945 | 29.452 | 1.00 | 56.60 |
| 1628 | C | SER | B | 337 | 43.758 | 64.917 | 30.624 | 1.00 | 55.17 |
| 1629 | O | SER | B | 337 | 43.338 | 64.619 | 31.748 | 1.00 | 54.55 |
| 1630 | CB | SER | B | 337 | 42.522 | 64.415 | 28.497 | 1.00 | 57.92 |
| 1631 | DC | SER | B | 337 | 41.244 | 64.111 | 29.013 | 1.00 | 64.70 |
| 1632 | N | ALA | B | 338 | 44.362 | 66.073 | 30.357 | 1.00 | 52.40 |
| 1633 | CA | ALA | B | 338 | 44.590 | 67.078 | 31.389 | 1.00 | 50.69 |
| 1634 | C | ALA | B | 338 | 44.300 | 68.491 | 30.908 | 1.00 | 50.25 |
| 1635 | O | ALA | B | 338 | 44.538 | 68.828 | 29.746 | 1.00 | 51.17 |
| 1636 | CB | ALA | B | 338 | 46.027 | 66.989 | 31.889 | 1.00 | 50.21 |
| 1637 | N | TYR | B | 339 | 43.792 | 69.319 | 31.816 | 1.00 | 48.22 |
| 1638 | CA | TYR | B | 339 | 43.461 | 70.701 | 31.495 | 1.00 | 46.82 |
| 1639 | C | TYR | B | 339 | 43.834 | 71.610 | 32.669 | 1.00 | 44.51 |
| 1640 | O | TYR | B | 339 | 43.711 | 71.228 | 33.832 | 1.00 | 42.27 |
| 1641 | CB | TYR | B | 339 | 41.962 | 70.835 | 31.185 | 1.00 | 50.52 |
| 1642 | CG | TYR | B | 339 | 41.402 | 69.731 | 30.307 | 1.00 | 58.40 |
| 1643 | CD1 | TYR | B | 339 | 41.155 | 68.456 | 30.826 | 1.00 | 61.23 |
| 1644 | CD2 | TYR | B | 339 | 41.144 | 69.952 | 28.949 | 1.00 | 60.99 |
| 1645 | CE1 | TYR | B | 339 | 40.671 | 67.426 | 30.019 | 1.00 | 62.71 |
| 1646 | CE2 | TYR | B | 339 | 40.657 | 68.926 | 28.131 | 1.00 | 63.53 |
| 1647 | CZ | TYR | B | 339 | 40.424 | 67.667 | 28.673 | 1.00 | 64.33 |
| 1648 | OH | TYR | B | 339 | 39.956 | 66.647 | 27.870 | 1.00 | 65.07 |
| 1649 | N | LEU | B | 340 | 44.309 | 72.807 | 32.354 | 1.00 | 41.90 |
| 1650 | CA | LEU | B | 340 | 44.695 | 73.769 | 33.372 | 1.00 | 41.06 |
| 1651 | C | LEU | B | 340 | 43.929 | 75.029 | 33.022 | 1.00 | 41.10 |
| 1652 | O | LEU | B | 340 | 43.953 | 75.464 | 31.876 | 1.00 | 43.45 |
| 1653 | CB | LEU | B | 340 | 46.208 | 74.017 | 33.321 | 1.00 | 36.93 |
| 1654 | CG | LEU | B | 340 | 46.830 | 74.911 | 34.399 | 1.00 | 35.91 |
| 1655 | CD1 | LEU | B | 340 | 46.500 | 74.371 | 35.786 | 1.00 | 34.60 |
| 1656 | CD2 | LEU | B | 340 | 48.336 | 74.979 | 34.194 | 1.00 | 35.51 |
| 1657 | N | SER | B | 341 | 43.232 | 75.610 | 33.992 | 1.00 | 39.82 |
| 1658 | CA | SER | B | 341 | 42.448 | 76.810 | 33.715 | 1.00 | 39.85 |
| 1659 | C | SER | B | 341 | 42.955 | 78.002 | 34.486 | 1.00 | 37.88 |
| 1660 | O | SER | B | 341 | 43.524 | 77.863 | 35.556 | 1.00 | 42.10 |
| 1661 | CB | SER | B | 341 | 40.973 | 76.575 | 34.058 | 1.00 | 39.34 |
| 1662 | OG | SER | B | 341 | 40.808 | 76.253 | 35.428 | 1.00 | 44.58 |
| 1663 | N | ARG | B | 342 | 42.732 | 79.185 | 33.942 | 1.00 | 36.98 |
| 1664 | CA | ARG | B | 342 | 43.163 | 80.413 | 34.583 | 1.00 | 35.20 |
| 1665 | C | ARG | B | 342 | 42.183 | 80.747 | 35.707 | 1.00 | 33.18 |
| 1666 | O | ARG | B | 342 | 41.157 | 80.092 | 35.848 | 1.00 | 33.16 |
| 1667 | CB | ARG | B | 342 | 43.206 | 81.530 | 33.534 | 1.00 | 38.57 |
| 1668 | CC | ARG | B | 342 | 44.219 | 81.279 | 32.411 | 1.00 | 39.10 |
| 1669 | CD | ARG | B | 342 | 44.236 | 82.409 | 31.388 | 1.00 | 43.21 |
| 1670 | NE | ARG | B | 342 | 43.056 | 82.369 | 30.530 | 1.00 | 45.40 |
| 1671 | CZ | ARG | B | 342 | 42.886 | 81.514 | 29.527 | 1.00 | 48.22 |
| 1672 | NH1 | ARG | B | 342 | 43.823 | 80.623 | 29.240 | 1.00 | 52.78 |
| 1673 | NH2 | ARG | B | 342 | 41.769 | 81.541 | 28.814 | 1.00 | 54.34 |
| 1674 | N | PRO | B | 343 | 42.488 | 81.757 | 36.530 | 1.00 | 33.03 |
| 1675 | CA | PRO | B | 343 | 41.560 | 82.103 | 37.619 | 1.00 | 34.14 |
| 1676 | C | PRO | B | 343 | 40.261 | 82.693 | 37.065 | 1.00 | 34.06 |
| 1677 | O | PRO | B | 343 | 40.259 | 83.298 | 35.994 | 1.00 | 35.44 |
| 1678 | CB | PRO | B | 343 | 42.334 | 83.152 | 38.433 | 1.00 | 33.07 |
| 1679 | CC | PRO | B | 343 | 43.789 | 82.900 | 38.075 | 1.00 | 35.06 |
| 1680 | CD | PRO | B | 343 | 43.715 | 82.571 | 36.601 | 1.00 | 33.27 |
| 1681 | N | SER | B | 344 | 39.155 | 82.520 | 37.775 | 1.00 | 32.17 |
| 1682 | CA | SER | B | 344 | 37.909 | 83.110 | 37.308 | 1.00 | 31.75 |
| 1683 | C | SER | B | 344 | 37.983 | 84.586 | 37.686 | 1.00 | 32.07 |
| 1684 | O | SER | B | 344 | 38.485 | 84.940 | 38.756 | 1.00 | 30.21 |
| 1685 | CB | SER | B | 344 | 36.693 | 82.456 | 37.982 | 1.00 | 32.20 |
| 1686 | OC | SER | B | 344 | 36.487 | 82.935 | 39.301 | 1.00 | 32.50 |
| 1687 | N | PRO | B | 345 | 37.512 | 85.473 | 36.800 | 1.00 | 32.31 |
| 1688 | CA | PRO | B | 345 | 37.561 | 86.899 | 37.125 | 1.00 | 30.84 |
| 1689 | C | PRO | B | 345 | 36.890 | 87.219 | 38.471 | 1.00 | 29.64 |
| 1690 | O | PRO | B | 345 | 37.344 | 88.094 | 39.209 | 1.00 | 30.14 |
| 1691 | CB | PRO | B | 345 | 36.859 | 87.545 | 35.926 | 1.00 | 30.96 |
| 1692 | CC | PRO | B | 345 | 37.299 | 86.659 | 34.793 | 1.00 | 33.18 |
| 1693 | CD | PRO | B | 345 | 37.143 | 85.261 | 35.387 | 1.00 | 33.65 |
| 1694 | N | PHE | B | 346 | 35.831 | 86.497 | 38.808 | 1.00 | 28.34 |
| 1695 | CA | PHE | B | 346 | 35.153 | 86.756 | 40.066 | 1.00 | 31.46 |
| 1696 | C | PHE | B | 346 | 36.078 | 86.515 | 41.269 | 1.00 | 32.46 |
| 1697 | O | PHE | B | 346 | 36.135 | 87.331 | 42.193 | 1.00 | 32.59 |
| 1698 | CB | PHE | B | 346 | 33.894 | 85.889 | 40.184 | 1.00 | 32.72 |
| 1699 | CC | PHE | B | 346 | 33.218 | 85.987 | 41.521 | 1.00 | 36.34 |
| 1700 | CD1 | PHE | B | 346 | 32.619 | 87.170 | 41.928 | 1.00 | 40.58 |
| 1701 | CD2 | PHE | B | 346 | 33.189 | 84.898 | 42.378 | 1.00 | 36.76 |
| 1702 | CE1 | PHE | B | 346 | 31.991 | 87.267 | 43.167 | 1.00 | 38.98 |
| 1703 | CR2 | PHE | B | 346 | 32.563 | 84.985 | 43.618 | 1.00 | 40.15 |
| 1704 | CZ | PHE | B | 346 | 31.963 | 86.175 | 44.014 | 1.00 | 38.60 |
| 1705 | N | ASP | B | 347 | 36.803 | 85.397 | 41.250 | 1.00 | 32.92 |
| 1706 | CA | ASP | B | 347 | 37.723 | 85.066 | 42.336 | 1.00 | 33.58 |
| 1707 | C | ASP | B | 347 | 38.896 | 86.045 | 42.367 | 1.00 | 34.26 |
| 1708 | O | ASP | B | 347 | 39.388 | 86.408 | 43.434 | 1.00 | 32.29 |
| 1709 | CB | ASP | B | 347 | 38.251 | 83.633 | 42.179 | 1.00 | 28.72 |
| 1710 | CG | ASP | B | 347 | 37.236 | 82.592 | 42.579 | 1.00 | 39.62 |
| 1711 | OD1 | ASP | B | 347 | 37.515 | 81.375 | 42.429 | 1.00 | 42.08 |
| 1712 | OD2 | ASP | B | 347 | 36.151 | 82.991 | 43.054 | 1.00 | 46.23 |
| 1713 | N | LEU | B | 348 | 39.324 | 86.480 | 41.189 | 1.00 | 34.31 |
| 1714 | CA | LEU | B | 348 | 40.441 | 87.403 | 41.064 | 1.00 | 37.70 |
| 1715 | C | LEU | B | 348 | 40.159 | 88.858 | 41.471 | 1.00 | 38.98 |
| 1716 | O | LEU | B | 348 | 40.949 | 89.463 | 42.198 | 1.00 | 38.11 |
| 1717 | CB | LEU | B | 348 | 40.959 | 87.380 | 39.618 | 1.00 | 40.66 |
| 1718 | CG | LEU | B | 348 | 42.159 | 88.267 | 39.260 | 1.00 | 41.99 |
| 1719 | CD1 | LEU | B | 348 | 43.388 | 87.862 | 40.095 | 1.00 | 42.77 |
| 1720 | CD2 | LEU | B | 348 | 42.469 | 88.124 | 37.759 | 1.00 | 41.00 |
| 1721 | N | PHE | B | 349 | 39.040 | 89.417 | 41.017 | 1.00 | 39.68 |
| 1722 | CA | PHE | B | 349 | 38.725 | 90.819 | 41.306 | 1.00 | 42.09 |
| 1723 | C | PHE | B | 349 | 37.717 | 91.134 | 42.405 | 1.00 | 45.09 |
| 1724 | O | PHE | B | 349 | 37.821 | 92.169 | 43.062 | 1.00 | 48.27 |
| 1725 | CB | PHE | B | 349 | 38.272 | 91.526 | 40.029 | 1.00 | 37.36 |
| 1726 | CC | PHE | B | 349 | 39.244 | 91.411 | 38.900 | 1.00 | 36.93 |
| 1727 | CD1 | PHE | B | 349 | 38.976 | 90.579 | 37.818 | 1.00 | 38.16 |
| 1728 | CD2 | PHE | B | 349 | 40.444 | 92.113 | 38.926 | 1.00 | 38.04 |
| 1729 | CE1 | PHE | B | 349 | 39.885 | 90.445 | 36.781 | 1.00 | 38.11 |
| 1730 | CE2 | PHE | B | 349 | 41.369 | 91.986 | 37.887 | 1.00 | 37.58 |
| 1731 | CZ | PHE | B | 349 | 41.089 | 91.151 | 36.817 | 1.00 | 40.39 |
| 1732 | N | ILE | B | 350 | 36.726 | 90.277 | 42.600 | 1.00 | 46.66 |
| 1733 | CA | ILE | B | 350 | 35.746 | 90.548 | 43.639 | 1.00 | 48.94 |
| 1734 | C | ILE | B | 350 | 36.200 | 89.905 | 44.950 | 1.00 | 51.15 |
| 1735 | O | ILE | B | 350 | 36.505 | 90.593 | 45.921 | 1.00 | 50.51 |
| 1736 | CB | ILE | B | 350 | 34.359 | 90.008 | 43.240 | 1.00 | 48.52 |
| 1737 | CG1 | ILE | B | 350 | 34.038 | 90.428 | 41.801 | 1.00 | 49.25 |
| 1738 | CG2 | ILE | B | 350 | 33.300 | 90.548 | 44.177 | 1.00 | 49.68 |
| 1739 | CD1 | ILE | B | 350 | 34.242 | 91.911 | 41.533 | 1.00 | 46.80 |
| 1740 | N | ARG | B | 351 | 36.265 | 88.579 | 44.945 | 1.00 | 52.64 |
| 1741 | CA | ARG | B | 351 | 36.669 | 87.793 | 46.100 | 1.00 | 53.76 |
| 1742 | C | ARG | B | 351 | 38.114 | 88.090 | 46.519 | 1.00 | 53.61 |
| 1743 | O | ARG | B | 351 | 38.436 | 88.094 | 47.703 | 1.00 | 53.55 |
| 1744 | CB | ARG | B | 351 | 36.505 | 86.313 | 45.759 | 1.00 | 56.64 |
| 1745 | CG | ARG | B | 351 | 36.016 | 85.434 | 46.894 | 1.00 | 62.18 |
| 1746 | CD | ARG | B | 351 | 35.073 | 84.375 | 46.348 | 1.00 | 66.96 |
| 1747 | NE | ARG | B | 351 | 34.958 | 83.211 | 47.219 | 1.00 | 71.13 |
| 1748 | CZ | ARG | B | 351 | 35.957 | 82.371 | 47.470 | 1.00 | 76.72 |
| 1749 | NH1 | ARG | B | 351 | 37.145 | 82.573 | 46.916 | 1.00 | 78.73 |
| 1750 | NE2 | ARG | B | 351 | 35.771 | 81.325 | 48.268 | 1.00 | 77.63 |
| 1751 | N | LYS | B | 352 | 38.974 | 88.347 | 45.540 | 1.00 | 53.86 |
| 1752 | CA | LYS | B | 352 | 40.385 | 88.646 | 45.788 | 1.00 | 54.12 |
| 1753 | C | LYS | B | 352 | 41.172 | 87.449 | 46.328 | 1.00 | 52.02 |
| 1754 | O | LYS | B | 352 | 42.078 | 87.604 | 47.146 | 1.00 | 51.77 |
| 1755 | CB | LYS | B | 352 | 40.525 | 89.825 | 46.753 | 1.00 | 57.77 |
| 1756 | CG | LYS | B | 352 | 39.911 | 91.124 | 46.254 | 1.00 | 62.15 |
| 1757 | CD | LYS | B | 352 | 40.161 | 92.252 | 47.254 | 1.00 | 71.06 |
| 1758 | CE | LYS | B | 352 | 39.351 | 93.502 | 46.929 | 1.00 | 74.25 |
| 1759 | NZ | LYS | B | 352 | 37.893 | 93.307 | 47.185 | 1.00 | 78.35 |
| 1760 | N | SER | B | 353 | 40.811 | 86.258 | 45.867 | 1.00 | 48.01 |
| 1761 | CA | SER | B | 353 | 41.488 | 85.032 | 46.268 | 1.00 | 45.29 |
| 1762 | C | SER | B | 353 | 41.446 | 84.056 | 45.084 | 1.00 | 40.67 |
| 1763 | O | SER | B | 353 | 40.725 | 83.054 | 45.106 | 1.00 | 40.76 |
| 1764 | CB | SER | B | 353 | 40.809 | 84.423 | 47.498 | 1.00 | 47.97 |
| 1765 | OG | SER | B | 353 | 39.445 | 84.152 | 47.247 | 1.00 | 56.20 |
| 1766 | N | PRO | B | 354 | 42.225 | 84.356 | 44.030 | 1.00 | 36.53 |
| 1767 | CA | PRO | B | 354 | 42.361 | 83.596 | 42.781 | 1.00 | 32.68 |
| 1768 | C | PRO | B | 354 | 42.940 | 82.201 | 42.961 | 1.00 | 31.88 |
| 1769 | O | PRO | B | 354 | 43.813 | 81.985 | 43.798 | 1.00 | 29.76 |
| 1770 | CB | PRO | B | 354 | 43.306 | 84.453 | 41.940 | 1.00 | 30.29 |
| 1771 | CG | PRO | B | 354 | 43.260 | 85.801 | 42.587 | 1.00 | 41.68 |
| 1772 | CD | PRO | B | 354 | 43.155 | 85.494 | 44.035 | 1.00 | 33.29 |
| 1773 | N | THR | B | 355 | 42.446 | 81.258 | 42.168 | 1.00 | 30.03 |
| 1774 | CA | THR | B | 355 | 42.956 | 79.896 | 42.198 | 1.00 | 29.70 |
| 1775 | C | THR | B | 355 | 43.022 | 79.364 | 40.774 | 1.00 | 30.82 |
| 1776 | O | THR | B | 355 | 42.302 | 79.827 | 39.887 | 1.00 | 30.67 |
| 1777 | CB | TNR | B | 355 | 42.042 | 78.931 | 42.964 | 1.00 | 32.14 |
| 1778 | OG1 | THR | B | 355 | 40.815 | 78.794 | 42.240 | 1.00 | 29.97 |
| 1779 | CG2 | THR | B | 355 | 41.763 | 79.425 | 44.393 | 1.00 | 31.76 |
| 1780 | N | ILE | B | 356 | 43.907 | 78.405 | 40.542 | 1.00 | 30.53 |
| 1781 | CA | ILE | B | 356 | 43.969 | 77.784 | 39.232 | 1.00 | 31.56 |
| 1782 | C | ILE | B | 356 | 43.740 | 76.321 | 39.525 | 1.00 | 31.79 |
| 1783 | O | ILE | B | 356 | 44.039 | 75.848 | 40.625 | 1.00 | 30.35 |
| 1784 | CB | ILE | B | 356 | 45.315 | 78.016 | 38.517 | 1.00 | 30.05 |
| 1785 | CG1 | ILE | B | 356 | 46.467 | 77.499 | 39.371 | 1.00 | 31.32 |
| 1786 | CG2 | ILE | B | 356 | 45.469 | 79.508 | 38.210 | 1.00 | 31.27 |
| 1787 | CD1 | ILE | B | 356 | 47.828 | 77.757 | 38.763 | 1.00 | 36.89 |
| 1788 | N | THR | B | 357 | 43.198 | 75.605 | 38.550 | 1.00 | 33.31 |
| 1789 | CA | THR | B | 357 | 42.881 | 74.205 | 38.751 | 1.00 | 33.68 |
| 1790 | C | THR | B | 357 | 43.403 | 73.318 | 37.644 | 1.00 | 35.47 |
| 1791 | O | THR | B | 357 | 43.228 | 73.607 | 36.459 | 1.00 | 36.65 |
| 1792 | CB | THR | B | 357 | 41.335 | 74.011 | 38.855 | 1.00 | 33.81 |
| 1793 | OG1 | THR | B | 357 | 40.844 | 74.683 | 40.023 | 1.00 | 33.71 |
| 1794 | CG2 | THR | B | 357 | 40.968 | 72.535 | 38.921 | 1.00 | 29.63 |
| 1795 | N | CYS | B | 358 | 44.034 | 72.228 | 38.048 | 1.00 | 35.41 |
| 1796 | CA | CYS | B | 358 | 44.561 | 71.254 | 37.110 | 1.00 | 38.12 |
| 1797 | C | CYS | B | 358 | 43.570 | 70.093 | 37.165 | 1.00 | 37.77 |
| 1798 | O | CYS | B | 358 | 43.395 | 69.473 | 38.210 | 1.00 | 36.67 |
| 1799 | CB | CYS | B | 358 | 45.945 | 70.789 | 37.568 | 1.00 | 40.37 |
| 1800 | SG | CYS | B | 358 | 46.871 | 69.769 | 36.374 | 1.00 | 49.83 |
| 1801 | N | LEU | B | 359 | 42.909 | 69.822 | 36.046 | 1.00 | 39.77 |
| 1802 | CA | LEU | B | 359 | 41.932 | 68.747 | 35.973 | 1.00 | 42.86 |
| 1803 | C | LEU | B | 359 | 42.432 | 67.593 | 35.123 | 1.00 | 46.01 |
| 1804 | O | LEU | B | 359 | 42.748 | 67.769 | 33.948 | 1.00 | 47.43 |
| 1805 | CB | LEU | B | 359 | 40.612 | 69.265 | 35.389 | 1.00 | 42.74 |
| 1806 | CC | LEU | B | 359 | 39.604 | 68.208 | 34.904 | 1.00 | 45.03 |
| 1807 | CD1 | LEU | B | 359 | 39.152 | 67.338 | 36.057 | 1.00 | 47.59 |
| 1808 | CD2 | LEU | B | 359 | 38.403 | 68.889 | 34.262 | 1.00 | 47.51 |
| 1809 | N | VAL | B | 360 | 42.493 | 66.411 | 35.721 | 1.00 | 48.26 |
| 1810 | CA | VAL | B | 360 | 42.931 | 65.220 | 35.010 | 1.00 | 52.34 |
| 1811 | C | VAL | B | 360 | 41.765 | 64.243 | 34.883 | 1.00 | 55.48 |
| 1812 | O | VAL | B | 360 | 41.092 | 63.928 | 35.866 | 1.00 | 55.57 |
| 1813 | CB | VAL | B | 360 | 44.100 | 64.527 | 35.747 | 1.00 | 53.36 |
| 1814 | CG1 | VAL | B | 360 | 44.481 | 63.240 | 35.029 | 1.00 | 51.62 |
| 1815 | CG2 | VAL | B | 360 | 45.299 | 65.467 | 35.816 | 1.00 | 51.77 |
| 1816 | N | VAL | B | 361 | 41.520 | 63.773 | 33.665 | 1.00 | 58.92 |
| 1817 | CA | VAL | B | 361 | 40.433 | 62.834 | 33.416 | 1.00 | 63.13 |
| 1818 | C | VAL | B | 361 | 40.993 | 61.506 | 32.926 | 1.00 | 67.77 |
| 1819 | O | VAL | B | 361 | 41.738 | 61.466 | 31.946 | 1.00 | 68.38 |
| 1820 | CB | VAL | B | 361 | 39.442 | 63.377 | 32.353 | 1.00 | 60.50 |
| 1821 | CG1 | VAL | B | 361 | 38.358 | 62.355 | 32.086 | 1.00 | 56.92 |
| 1822 | CG2 | VAL | B | 361 | 38.814 | 64.671 | 32.830 | 1.00 | 56.10 |
| 1823 | N | ASP | B | 362 | 40.635 | 60.426 | 33.615 | 1.00 | 72.90 |
| 1824 | CA | ASP | B | 362 | 41.098 | 59.085 | 33.259 | 1.00 | 78.30 |
| 1825 | C | ASP | B | 362 | 39.904 | 58.244 | 32.816 | 1.00 | 81.68 |
| 1826 | O | ASP | B | 362 | 39.168 | 57.709 | 33.648 | 1.00 | 81.72 |
| 1827 | CB | ASP | B | 362 | 41.771 | 58.415 | 34.462 | 1.00 | 80.22 |
| 1828 | CG | ASP | B | 362 | 42.530 | 57.156 | 34.080 | 1.00 | 84.67 |
| 1829 | OD1 | ASP | B | 362 | 42.113 | 56.478 | 33.117 | 1.00 | 87.54 |
| 1830 | OD2 | ASP | B | 362 | 43.537 | 56.837 | 34.749 | 1.00 | 87.19 |
| 1831 | N | LEU | B | 363 | 39.723 | 58.126 | 31.503 | 1.00 | 85.84 |
| 1832 | CA | LEU | B | 363 | 38.614 | 57.366 | 30.935 | 1.00 | 89.95 |
| 1833 | C | LEU | B | 363 | 38.562 | 55.923 | 31.428 | 1.00 | 92.20 |
| 1834 | O | LEU | B | 363 | 37.483 | 55.386 | 31.668 | 1.00 | 92.55 |
| 1835 | CB | LEU | B | 363 | 38.694 | 57.390 | 29.409 | 1.00 | 91.66 |
| 1836 | CG | LEU | B | 363 | 38.719 | 58.777 | 28.758 | 1.00 | 92.93 |
| 1837 | CD1 | LEU | B | 363 | 38.813 | 58.624 | 27.246 | 1.00 | 95.41 |
| 1838 | CD2 | LEU | B | 363 | 37.471 | 59.551 | 29.137 | 1.00 | 92.73 |
| 1839 | N | ALA | B | 364 | 39.726 | 55.296 | 31.573 | 1.00 | 94.76 |
| 1840 | CA | ALA | B | 364 | 39.801 | 53.916 | 32.051 | 1.00 | 97.66 |
| 1841 | C | ALA | B | 364 | 40.794 | 53.831 | 33.210 | 1.00 | 99.72 |
| 1842 | O | ALA | B | 364 | 41.998 | 53.996 | 33.021 | 1.00 | 100.44 |
| 1843 | CB | ALA | B | 364 | 40.235 | 52.996 | 30.921 | 1.00 | 97.99 |
| 1844 | N | PRO | B | 365 | 40.297 | 53.577 | 34.429 | 1.00 | 101.15 |
| 1845 | CA | PRO | B | 365 | 41.173 | 53.484 | 35.602 | 1.00 | 102.25 |
| 1846 | C | PRO | B | 365 | 41.895 | 52.163 | 35.855 | 1.00 | 103.43 |
| 1847 | O | PRO | B | 365 | 41.274 | 51.103 | 35.934 | 1.00 | 104.19 |
| 1848 | CB | PRO | B | 365 | 40.237 | 53.829 | 36.761 | 1.00 | 101.91 |
| 1849 | CC | PRO | B | 365 | 39.121 | 54.617 | 36.098 | 1.00 | 102.27 |
| 1850 | CD | PRO | B | 365 | 38.910 | 53.833 | 34.846 | 1.00 | 101.40 |
| 1851 | N | SER | B | 366 | 43.215 | 52.256 | 35.979 | 1.00 | 104.18 |
| 1852 | CA | SER | B | 366 | 44.088 | 51.132 | 36.299 | 1.00 | 104.43 |
| 1853 | C | SER | B | 366 | 44.573 | 51.676 | 37.637 | 1.00 | 104.05 |
| 1854 | O | SER | B | 366 | 44.888 | 52.864 | 37.727 | 1.00 | 104.36 |
| 1855 | CB | SER | B | 366 | 45.249 | 51.055 | 35.312 | 1.00 | 105.50 |
| 1856 | OG | SER | B | 366 | 46.040 | 52.223 | 35.413 | 1.00 | 106.70 |
| 1857 | N | LYS | B | 367 | 44.645 | 50.861 | 38.682 | 1.00 | 103.19 |
| 1858 | CA | LYS | B | 367 | 45.041 | 51.462 | 39.944 | 1.00 | 102.08 |
| 1859 | C | LYS | B | 367 | 46.491 | 51.630 | 40.367 | 1.00 | 100.74 |
| 1860 | O | LYS | B | 367 | 47.234 | 50.683 | 40.640 | 1.00 | 101.05 |
| 1861 | CB | LYS | B | 367 | 44.236 | 50.868 | 41.102 | 1.00 | 103.30 |
| 1862 | CC | LYS | B | 367 | 44.343 | 49.384 | 41.345 | 1.00 | 105.00 |
| 1863 | CD | LYS | B | 367 | 43.374 | 49.046 | 42.467 | 1.00 | 104.80 |
| 1864 | CE | LYS | B | 367 | 43.467 | 47.610 | 42.930 | 1.00 | 104.70 |
| 1865 | NZ | LYS | B | 367 | 42.524 | 47.386 | 44.066 | 1.00 | 105.18 |
| 1866 | N | CLY | B | 368 | 46.839 | 52.909 | 40.422 | 1.00 | 98.70 |
| 1867 | CA | GLY | B | 368 | 48.136 | 53.408 | 40.828 | 1.00 | 95.62 |
| 1868 | C | GLY | B | 368 | 47.700 | 54.801 | 41.244 | 1.00 | 93.12 |
| 1869 | O | GLY | B | 368 | 46.538 | 55.143 | 41.035 | 1.00 | 93.17 |
| 1870 | N | THR | B | 369 | 48.569 | 55.617 | 41.823 | 1.00 | 90.15 |
| 1871 | CA | THR | B | 369 | 48.125 | 56.948 | 42.220 | 1.00 | 86.41 |
| 1872 | C | THR | B | 369 | 48.420 | 57.977 | 41.136 | 1.00 | 83.04 |
| 1873 | O | THR | B | 369 | 49.402 | 57.854 | 40.401 | 1.00 | 82.54 |
| 1874 | CB | THR | B | 369 | 48.792 | 57.397 | 43.541 | 1.00 | 87.73 |
| 1875 | OG1 | THR | B | 369 | 50.217 | 57.422 | 43.379 | 1.00 | 88.60 |
| 1876 | CG2 | THR | B | 369 | 48.423 | 56.443 | 44.671 | 1.00 | 86.64 |
| 1877 | N | VAL | B | 370 | 47.552 | 58.980 | 41.027 | 1.00 | 79.07 |
| 1878 | CA | VAL | B | 370 | 47.729 | 60.041 | 40.042 | 1.00 | 74.46 |
| 1879 | C | VAL | B | 370 | 48.275 | 61.261 | 40.768 | 1.00 | 72.21 |
| 1880 | O | VAL | B | 370 | 47.602 | 61.838 | 41.618 | 1.00 | 71.35 |
| 1881 | CB | VAL | B | 370 | 46.401 | 60.420 | 39.368 | 1.00 | 73.33 |
| 1882 | CD1 | VAL | B | 370 | 46.667 | 61.346 | 38.196 | 1.00 | 70.92 |
| 1883 | CG2 | VAL | B | 370 | 45.674 | 59.173 | 38.915 | 1.00 | 69.77 |
| 1884 | N | ASN | B | 371 | 49.498 | 61.653 | 40.426 | 1.00 | 70.13 |
| 1885 | CA | ASN | B | 371 | 50.137 | 62.791 | 41.073 | 1.00 | 68.15 |
| 1886 | C | ASN | B | 371 | 50.157 | 64.079 | 40.262 | 1.00 | 65.65 |
| 1887 | O | ASN | B | 371 | 50.352 | 64.067 | 39.046 | 1.00 | 64.64 |
| 1888 | CB | ASN | B | 371 | 51.564 | 62.421 | 41.469 | 1.00 | 70.12 |
| 1889 | CG | ASN | B | 371 | 51.605 | 61.330 | 42.508 | 1.00 | 73.56 |
| 1890 | OD1 | ASN | B | 371 | 51.153 | 61.519 | 43.638 | 1.00 | 75.10 |
| 1891 | ND2 | ASN | B | 371 | 52.137 | 60.173 | 42.131 | 1.00 | 74.76 |
| 1892 | N | LEU | B | 372 | 49.949 | 65.192 | 40.959 | 1.00 | 62.66 |
| 1893 | CA | LEU | B | 372 | 49.959 | 66.512 | 40.346 | 1.00 | 59.53 |
| 1894 | C | LEU | B | 372 | 50.927 | 67.374 | 41.137 | 1.00 | 56.68 |
| 1895 | O | LEU | B | 372 | 50.788 | 67.529 | 42.347 | 1.00 | 56.70 |
| 1896 | CB | LEU | B | 372 | 48.560 | 67.141 | 40.372 | 1.00 | 59.44 |
| 1897 | CG | LEU | B | 372 | 47.428 | 66.383 | 39.678 | 1.00 | 60.55 |
| 1898 | CU | LEU | B | 372 | 46.214 | 67.277 | 39.579 | 1.00 | 61.28 |
| 1899 | CD2 | LEU | B | 372 | 47.861 | 65.956 | 38.293 | 1.00 | 62.83 |
| 1900 | N | THR | B | 373 | 51.911 | 67.931 | 40.446 | 1.00 | 53.76 |
| 1901 | CA | THR | B | 373 | 52.915 | 68.766 | 41.085 | 1.00 | 51.64 |
| 1902 | C | THR | B | 373 | 52.850 | 70.180 | 40.512 | 1.00 | 48.20 |
| 1903 | O | THR | B | 373 | 52.801 | 70.355 | 39.296 | 1.00 | 46.65 |
| 1904 | CB | THR | B | 373 | 54.325 | 68.201 | 40.832 | 1.00 | 53.15 |
| 1905 | CG1 | THR | B | 373 | 54.324 | 66.789 | 41.073 | 1.00 | 57.77 |
| 1906 | CG2 | THR | B | 373 | 55.339 | 68.871 | 41.747 | 1.00 | 53.75 |
| 1907 | N | TRP | B | 374 | 52.851 | 71.181 | 41.385 | 1.00 | 45.34 |
| 1908 | CA | TRP | B | 374 | 52.799 | 72.575 | 40.942 | 1.00 | 44.94 |
| 1909 | C | TRP | B | 374 | 54.161 | 73.259 | 41.041 | 1.00 | 43.68 |
| 1910 | O | TRP | B | 374 | 54.945 | 72.964 | 41.938 | 1.00 | 42.67 |
| 1911 | CB | TRP | B | 374 | 51.805 | 73.391 | 41.784 | 1.00 | 41.63 |
| 1912 | CG | TRP | B | 374 | 50.371 | 72.977 | 41.657 | 1.00 | 41.50 |
| 1913 | CD1 | TRP | B | 374 | 49.688 | 72.124 | 42.477 | 1.00 | 39.67 |
| 1914 | CD2 | TRP | B | 374 | 49.441 | 73.395 | 40.648 | 1.00 | 35.69 |
| 1915 | NE1 | TRP | B | 374 | 48.392 | 71.988 | 42.041 | 1.00 | 35.06 |
| 1916 | CE2 | TRP | B | 374 | 48.214 | 72.756 | 40.922 | 1.00 | 34.53 |
| 1917 | CE3 | TRP | B | 374 | 49.528 | 74.247 | 39.542 | 1.00 | 32.37 |
| 1918 | CZ2 | TRP | B | 374 | 47.079 | 72.943 | 40.129 | 1.00 | 35.43 |
| 1919 | CZ3 | TRP | B | 374 | 48.401 | 74.434 | 38.751 | 1.00 | 36.11 |
| 1920 | CH2 | TRP | B | 374 | 47.190 | 73.782 | 39.050 | 1.00 | 37.51 |
| 1921 | N | SER | B | 375 | 54.430 | 74.179 | 40.119 | 1.00 | 43.53 |
| 1922 | CA | SER | B | 375 | 55.678 | 74.934 | 40.140 | 1.00 | 43.57 |
| 1923 | C | SER | B | 375 | 55.511 | 76.270 | 39.438 | 1.00 | 42.59 |
| 1924 | O | SER | B | 375 | 54.669 | 76.411 | 38.550 | 1.00 | 43.68 |
| 1925 | CB | SER | B | 375 | 56.820 | 74.153 | 39.474 | 1.00 | 45.03 |
| 1926 | OG | SER | B | 375 | 56.691 | 74.118 | 38.066 | 1.00 | 45.66 |
| 1927 | N | ARG | B | 376 | 56.299 | 77.251 | 39.866 | 1.00 | 40.79 |
| 1928 | CA | ARG | B | 376 | 56.279 | 78.569 | 39.266 | 1.00 | 40.12 |
| 1929 | C | ARG | B | 376 | 57.498 | 78.689 | 38.351 | 1.00 | 40.59 |
| 1930 | O | ARG | B | 376 | 58.618 | 78.393 | 38.752 | 1.00 | 40.90 |
| 1931 | CB | ARG | B | 376 | 56.345 | 79.653 | 40.334 | 1.00 | 40.03 |
| 1932 | CG | ARG | B | 376 | 55.132 | 79.745 | 41.237 | 1.00 | 43.20 |
| 1933 | CD | ARG | B | 376 | 54.883 | 81.198 | 41.599 | 1.00 | 41.05 |
| 1934 | NE | ARG | B | 376 | 55.171 | 81.480 | 42.992 | 1.00 | 50.71 |
| 1935 | CZ | ARG | B | 376 | 55.300 | 82.705 | 43.488 | 1.00 | 53.97 |
| 1936 | NE1 | ARG | B | 376 | 55.172 | 83.758 | 42.693 | 1.00 | 51.88 |
| 1937 | NH2 | ARG | B | 376 | 55.542 | 82.874 | 44.782 | 1.00 | 55.55 |
| 1938 | N | ALA | B | 377 | 57.281 | 79.129 | 37.122 | 1.00 | 40.20 |
| 1939 | CA | ALA | B | 377 | 58.382 | 79.273 | 36.181 | 1.00 | 40.25 |
| 1940 | C | ALA | B | 377 | 59.500 | 80.130 | 36.786 | 1.00 | 40.38 |
| 1941 | O | ALA | B | 377 | 60.657 | 79.979 | 36.422 | 1.00 | 41.16 |
| 1942 | CB | ALA | B | 377 | 57.879 | 79.903 | 34.878 | 1.00 | 33.12 |
| 1943 | N | SER | B | 378 | 59.144 | 81.015 | 37.714 | 1.00 | 40.21 |
| 1944 | CA | SER | B | 378 | 60.109 | 81.903 | 38.354 | 1.00 | 41.95 |
| 1945 | C | SER | B | 378 | 60.906 | 81.188 | 39.436 | 1.00 | 44.92 |
| 1946 | O | SER | B | 378 | 61.866 | 81.744 | 39.966 | 1.00 | 44.28 |
| 1947 | CB | SER | B | 378 | 59.401 | 83.096 | 38.992 | 1.00 | 40.91 |
| 1948 | OG | SER | B | 378 | 58.737 | 82.693 | 40.180 | 1.00 | 40.09 |
| 1949 | N | GLY | B | 379 | 60.493 | 79.967 | 39.768 | 1.00 | 46.93 |
| 1950 | CA | GLY | B | 379 | 61.181 | 79.195 | 40.787 | 1.00 | 49.77 |
| 1951 | C | GLY | B | 379 | 60.716 | 79.512 | 42.196 | 1.00 | 50.60 |
| 1952 | O | GLY | B | 379 | 61.024 | 78.783 | 43.142 | 1.00 | 52.95 |
| 1953 | N | LYS | B | 380 | 59.974 | 80.605 | 42.345 | 1.00 | 53.72 |
| 1954 | CA | LYS | B | 380 | 59.478 | 80.988 | 43.657 | 1.00 | 54.13 |
| 1955 | C | LYS | B | 380 | 58.564 | 79.911 | 44.255 | 1.00 | 48.16 |
| 1956 | O | LYS | B | 380 | 58.087 | 79.008 | 43.556 | 1.00 | 45.65 |
| 1957 | CB | LYS | B | 380 | 58.752 | 82.338 | 43.583 | 1.00 | 62.44 |
| 1958 | CG | LYS | B | 380 | 59.692 | 83.522 | 43.390 | 1.00 | 67.72 |
| 1959 | CD | LYS | B | 380 | 58.975 | 84.852 | 43.550 | 1.00 | 69.32 |
| 1960 | CE | LYS | B | 380 | 59.956 | 86.013 | 43.481 | 1.00 | 73.14 |
| 1961 | NZ | LYS | B | 380 | 59.280 | 87.333 | 43.633 | 1.00 | 77.37 |
| 1962 | N | PRO | B | 381 | 58.323 | 79.986 | 45.571 | 1.00 | 56.07 |
| 1963 | CA | PRO | B | 381 | 57.472 | 79.014 | 46.265 | 1.00 | 56.46 |
| 1964 | C | PRO | B | 381 | 55.993 | 79.065 | 45.875 | 1.00 | 54.32 |
| 1965 | O | PRO | B | 381 | 55.446 | 80.133 | 45.589 | 1.00 | 56.76 |
| 1966 | CB | PRO | B | 381 | 57.664 | 79.375 | 47.740 | 1.00 | 60.70 |
| 1967 | CG | PRO | B | 381 | 58.978 | 80.119 | 47.767 | 1.00 | 62.01 |
| 1968 | CD | PRO | B | 381 | 58.905 | 80.945 | 46.526 | 1.00 | 59.24 |
| 1969 | N | VAL | B | 382 | 55.356 | 77.899 | 45.866 | 1.00 | 55.74 |
| 1970 | CA | VAL | B | 382 | 53.931 | 77.805 | 45.569 | 1.00 | 55.58 |
| 1971 | C | VAL | B | 382 | 53.247 | 77.408 | 46.880 | 1.00 | 55.56 |
| 1972 | O | VAL | B | 382 | 53.848 | 76.734 | 47.720 | 1.00 | 55.22 |
| 1973 | CB | VAL | B | 382 | 53.620 | 76.726 | 44.493 | 1.00 | 55.39 |
| 1974 | CD1 | VAL | B | 382 | 54.404 | 77.008 | 43.222 | 1.00 | 55.94 |
| 1975 | CG2 | VAL | B | 382 | 53.936 | 75.339 | 45.025 | 1.00 | 53.06 |
| 1976 | N | ASN | B | 383 | 52.000 | 77.827 | 47.055 | 1.00 | 54.38 |
| 1977 | CA | ASN | B | 383 | 51.246 | 77.510 | 48.264 | 1.00 | 54.63 |
| 1978 | C | ASN | B | 383 | 50.757 | 76.068 | 48.290 | 1.00 | 53.85 |
| 1979 | O | ASN | B | 383 | 50.970 | 75.297 | 47.354 | 1.00 | 52.58 |
| 1980 | CB | ASN | B | 383 | 50.034 | 78.429 | 48.382 | 1.00 | 59.07 |
| 1981 | CG | ASN | B | 383 | 50.414 | 79.883 | 48.456 | 1.00 | 64.60 |
| 1982 | OD1 | ASN | B | 383 | 49.576 | 80.764 | 48.269 | 1.00 | 70.14 |
| 1983 | ND2 | ASN | B | 383 | 51.685 | 80.150 | 48.738 | 1.00 | 71.07 |
| 1984 | N | HIS | B | 384 | 50.094 | 75.710 | 49.381 | 1.00 | 53.46 |
| 1985 | CA | HIS | B | 384 | 49.543 | 74.371 | 49.526 | 1.00 | 52.22 |
| 1986 | C | HIS | B | 384 | 48.298 | 74.274 | 48.640 | 1.00 | 49.10 |
| 1987 | O | HIS | B | 384 | 47.495 | 75.200 | 48.585 | 1.00 | 45.53 |
| 1988 | CB | HIS | B | 384 | 49.183 | 74.111 | 50.993 | 1.00 | 59.11 |
| 1989 | CG | HIS | B | 384 | 50.377 | 73.924 | 51.878 | 1.00 | 62.16 |
| 1990 | ND1 | HIS | B | 384 | 51.243 | 72.862 | 51.739 | 1.00 | 69.15 |
| 1991 | CD2 | HIS | B | 384 | 50.855 | 74.669 | 52.903 | 1.00 | 70.28 |
| 1992 | CE1 | HIS | B | 384 | 52.206 | 72.960 | 52.639 | 1.00 | 75.01 |
| 1993 | NE2 | HIS | B | 384 | 51.995 | 74.047 | 53.358 | 1.00 | 74.58 |
| 1994 | N | SER | B | 385 | 48.146 | 73.152 | 47.949 | 1.00 | 45.07 |
| 1995 | CA | SER | B | 385 | 47.017 | 72.959 | 47.060 | 1.00 | 44.61 |
| 1996 | C | SER | B | 385 | 45.973 | 71.983 | 47.604 | 1.00 | 44.58 |
| 1997 | O | SER | B | 385 | 46.244 | 71.220 | 48.521 | 1.00 | 44.21 |
| 1998 | CB | SER | B | 385 | 47.524 | 72.487 | 45.691 | 1.00 | 44.61 |
| 1999 | DC | SER | B | 385 | 48.310 | 71.307 | 45.789 | 1.00 | 41.13 |
| 2000 | N | THR | B | 386 | 44.773 | 72.025 | 47.035 | 1.00 | 44.32 |
| 2001 | CA | THR | B | 386 | 43.684 | 71.150 | 47.446 | 1.00 | 44.35 |
| 2002 | C | THR | B | 386 | 43.444 | 70.113 | 46.363 | 1.00 | 44.09 |
| 2003 | O | THR | B | 386 | 43.274 | 70.451 | 45.189 | 1.00 | 44.06 |
| 2004 | CB | THR | B | 386 | 42.374 | 71.936 | 47.680 | 1.00 | 45.20 |
| 2005 | OG1 | THR | B | 386 | 42.555 | 72.856 | 48.762 | 1.00 | 51.89 |
| 2006 | CG2 | THR | B | 386 | 41.232 | 70.984 | 48.036 | 1.00 | 47.20 |
| 2007 | N | ARG | B | 387 | 43.424 | 68.851 | 46.774 | 1.00 | 44.39 |
| 2008 | CA | ARG | B | 387 | 43.222 | 67.730 | 45.867 | 1.00 | 46.68 |
| 2009 | C | ARG | B | 387 | 41.837 | 67.080 | 46.030 | 1.00 | 47.05 |
| 2010 | O | ARG | B | 387 | 41.372 | 66.850 | 47.149 | 1.00 | 46.65 |
| 2011 | CB | ARG | B | 387 | 44.316 | 66.694 | 46.121 | 1.00 | 48.13 |
| 2012 | CG | ARG | B | 387 | 44.270 | 65.488 | 45.210 | 1.00 | 52.77 |
| 2013 | CD | ARG | B | 387 | 45.106 | 64.353 | 45.780 | 1.00 | 56.60 |
| 2014 | NE | ARG | B | 387 | 44.978 | 63.141 | 44.979 | 1.00 | 60.65 |
| 2015 | CZ | ARG | B | 387 | 45.683 | 62.895 | 43.882 | 1.00 | 61.97 |
| 2016 | NH1 | ARG | B | 387 | 46.578 | 63.779 | 43.464 | 1.00 | 64.45 |
| 2017 | NH2 | ARG | B | 387 | 45.469 | 61.781 | 43.189 | 1.00 | 60.54 |
| 2018 | N | LYS | B | 388 | 41.185 | 66.780 | 44.910 | 1.00 | 46.93 |
| 2019 | CA | LYS | B | 388 | 39.870 | 66.143 | 44.933 | 1.00 | 47.51 |
| 2020 | C | LYS | B | 388 | 39.823 | 64.970 | 43.957 | 1.00 | 48.33 |
| 2021 | O | LYS | B | 388 | 40.199 | 65.107 | 42.794 | 1.00 | 46.08 |
| 2022 | CB | LYS | B | 388 | 38.777 | 67.148 | 44.563 | 1.00 | 46.64 |
| 2023 | CC | LYS | B | 388 | 38.691 | 68.326 | 45.497 | 1.00 | 52.75 |
| 2024 | CD | LYS | B | 388 | 37.861 | 69.446 | 44.905 | 1.00 | 58.62 |
| 2025 | CE | LYS | B | 388 | 38.062 | 70.737 | 45.682 | 1.00 | 62.49 |
| 2026 | NZ | LYS | B | 388 | 37.396 | 71.881 | 44.999 | 1.00 | 67.33 |
| 2027 | N | GLU | B | 389 | 39.364 | 63.820 | 44.449 | 1.00 | 50.07 |
| 2028 | CA | GLU | B | 389 | 39.243 | 62.602 | 43.646 | 1.00 | 51.86 |
| 2029 | C | GLU | B | 389 | 37.793 | 62.126 | 43.638 | 1.00 | 52.81 |
| 2030 | O | GLU | B | 389 | 37.192 | 61.917 | 44.695 | 1.00 | 49.21 |
| 2031 | CB | CLO | B | 389 | 40.126 | 61.494 | 44.223 | 1.00 | 54.67 |
| 2032 | CC | GLU | B | 389 | 41.616 | 61.710 | 44.044 | 1.00 | 65.65 |
| 2033 | CD | GLU | B | 389 | 42.441 | 60.608 | 44.688 | 1.00 | 72.66 |
| 2034 | GE1 | GLU | B | 389 | 42.151 | 59.418 | 44.429 | 1.00 | 75.92 |
| 2035 | OE2 | GLU | B | 389 | 43.380 | 60.931 | 45.450 | 1.00 | 75.78 |
| 2036 | N | GLU | B | 390 | 37.229 | 61.948 | 42.449 | 1.00 | 56.06 |
| 2037 | CA | GLU | B | 390 | 35.849 | 61.500 | 42.346 | 1.00 | 61.17 |
| 2038 | C | GLU | B | 390 | 35.629 | 60.454 | 41.265 | 1.00 | 64.70 |
| 2039 | O | GLU | B | 390 | 36.072 | 60.598 | 40.123 | 1.00 | 64.01 |
| 2040 | CB | GLU | B | 390 | 34.927 | 62.692 | 42.107 | 1.00 | 64.35 |
| 2041 | CC | GLU | B | 390 | 33.479 | 62.317 | 41.867 | 1.00 | 74.80 |
| 2042 | CD | GLU | B | 390 | 32.548 | 63.509 | 41.979 | 1.00 | 85.97 |
| 2043 | OE1 | GLU | B | 390 | 32.822 | 64.547 | 41.336 | 1.00 | 92.60 |
| 2044 | OE2 | GLU | B | 390 | 31.541 | 63.403 | 42.712 | 1.00 | 92.18 |
| 2045 | N | LYS | B | 391 | 34.921 | 59.396 | 41.639 | 1.00 | 68.18 |
| 2046 | CA | LYS | B | 391 | 34.640 | 58.312 | 40.712 | 1.00 | 71.58 |
| 2047 | C | LYS | B | 391 | 33.215 | 58.302 | 40.180 | 1.00 | 76.11 |
| 2048 | O | LYS | B | 391 | 32.265 | 58.673 | 40.869 | 1.00 | 75.89 |
| 2049 | CB | LYS | B | 391 | 34.946 | 56.973 | 41.372 | 1.00 | 67.28 |
| 2050 | N | GLN | B | 392 | 33.092 | 57.876 | 38.932 | 1.00 | 81.85 |
| 2051 | CA | GLN | B | 392 | 31.814 | 57.746 | 38.254 | 1.00 | 88.38 |
| 2052 | C | GLN | B | 392 | 32.026 | 56.482 | 37.443 | 1.00 | 92.30 |
| 2053 | O | GLN | B | 392 | 32.752 | 56.500 | 36.452 | 1.00 | 93.25 |
| 2054 | CB | GLN | B | 392 | 31.555 | 58.938 | 37.323 | 1.00 | 85.87 |
| 2055 | CG | CLN | B | 392 | 30.422 | 59.848 | 37.782 | 1.00 | 90.76 |
| 2056 | CD | GLN | B | 392 | 30.027 | 60.873 | 36.731 | 1.00 | 93.39 |
| 2057 | OE1 | GLN | B | 392 | 29.687 | 60.524 | 35.598 | 1.00 | 92.93 |
| 2058 | NE2 | GLN | B | 392 | 30.062 | 62.147 | 37.107 | 1.00 | 93.97 |
| 2059 | N | ARG | B | 393 | 31.426 | 55.379 | 37.880 | 1.00 | 96.24 |
| 2060 | CA | ARG | B | 393 | 31.589 | 54.108 | 37.184 | 1.00 | 100.53 |
| 2061 | C | ARG | B | 393 | 31.250 | 54.188 | 35.697 | 1.00 | 102.30 |
| 2062 | O | ARG | B | 393 | 31.273 | 53.178 | 34.993 | 1.00 | 101.15 |
| 2063 | CB | ARG | B | 393 | 30.747 | 53.021 | 37.855 | 1.00 | 106.99 |
| 2064 | CG | ARG | B | 393 | 29.251 | 53.196 | 37.704 | 1.00 | 109.77 |
| 2065 | CD | ARG | B | 393 | 28.521 | 52.107 | 38.464 | 1.00 | 115.73 |
| 2066 | NE | ARG | B | 393 | 27.095 | 52.085 | 38.165 | 1.00 | 119.73 |
| 2067 | CZ | ARG | B | 393 | 26.227 | 51.267 | 38.750 | 1.00 | 121.60 |
| 2068 | NH1 | ARG | B | 393 | 26.645 | 50.406 | 39.670 | 1.00 | 121.59 |
| 2069 | NH2 | ARG | B | 393 | 24.945 | 51.308 | 38.416 | 1.00 | 121.49 |
| 2070 | N | ASM | B | 394 | 30.942 | 55.391 | 35.224 | 1.00 | 103.98 |
| 2071 | CA | ASN | B | 394 | 30.630 | 55.595 | 33.819 | 1.00 | 106.60 |
| 2072 | C | ASN | B | 394 | 31.918 | 55.325 | 33.049 | 1.00 | 106.89 |
| 2073 | O | ASN | B | 394 | 31.950 | 54.516 | 32.122 | 1.00 | 108.55 |
| 2074 | CB | ASN | B | 394 | 30.173 | 57.036 | 33.580 | 1.00 | 108.97 |
| 2075 | CG | ASN | B | 394 | 29.709 | 57.274 | 32.153 | 1.00 | 112.83 |
| 2076 | OD1 | ASN | B | 394 | 29.459 | 58.410 | 31.752 | 1.00 | 114.53 |
| 2077 | ND2 | ASN | B | 394 | 29.584 | 56.198 | 31.381 | 1.00 | 114.40 |
| 2078 | N | CLY | B | 395 | 32.984 | 56.007 | 33.452 | 1.00 | 105.53 |
| 2079 | CA | CLY | B | 395 | 34.269 | 55.829 | 32.802 | 1.00 | 101.15 |
| 2080 | C | CLY | B | 395 | 35.177 | 57.015 | 33.050 | 1.00 | 97.82 |
| 2081 | O | CLY | B | 395 | 35.879 | 57.475 | 32.148 | 1.00 | 99.41 |
| 2082 | N | THR | B | 396 | 35.177 | 57.516 | 34.278 | 1.00 | 94.50 |
| 2083 | CA | ThR | B | 396 | 36.011 | 58.663 | 34.578 | 1.00 | 89.02 |
| 2084 | C | THR | B | 396 | 36.426 | 58.837 | 36.029 | 1.00 | 83.93 |
| 2085 | O | THR | B | 396 | 35.600 | 59.123 | 36.896 | 1.00 | 83.27 |
| 2086 | CB | THR | B | 396 | 35.322 | 59.975 | 34.130 | 1.00 | 92.03 |
| 2087 | OC1 | THR | B | 396 | 35.116 | 59.952 | 32.712 | 1.00 | 94.17 |
| 2088 | CG2 | THR | B | 396 | 36.179 | 61.186 | 34.504 | 1.00 | 95.48 |
| 2089 | N | LEU | B | 397 | 37.718 | 58.658 | 36.279 | 1.00 | 79.69 |
| 2090 | CA | LEU | B | 397 | 38.280 | 58.871 | 37.600 | 1.00 | 74.75 |
| 2091 | C | LEU | B | 397 | 38.773 | 60.305 | 37.496 | 1.00 | 71.50 |
| 2092 | O | LEU | B | 397 | 39.880 | 60.548 | 37.019 | 1.00 | 74.81 |
| 2093 | CB | LEU | B | 397 | 39.480 | 57.960 | 37.860 | 1.00 | 75.72 |
| 2094 | CG | LEU | B | 397 | 40.350 | 58.412 | 39.047 | 1.00 | 70.67 |
| 2095 | CD1 | LEU | B | 397 | 39.742 | 57.932 | 40.362 | 1.00 | 69.05 |
| 2096 | CD2 | LEU | B | 397 | 41.768 | 57.877 | 38.887 | 1.00 | 72.96 |
| 2097 | N | THR | B | 398 | 37.941 | 61.252 | 37.905 | 1.00 | 67.61 |
| 2098 | CA | THR | B | 398 | 38.314 | 62.655 | 37.848 | 1.00 | 61.30 |
| 2099 | C | THR | B | 398 | 39.207 | 63.041 | 39.024 | 1.00 | 56.61 |
| 2100 | O | THR | B | 398 | 38.974 | 62.640 | 40.166 | 1.00 | 54.95 |
| 2101 | CB | THR | B | 398 | 37.070 | 63.556 | 37.852 | 1.00 | 61.75 |
| 2102 | OC1 | THR | B | 398 | 36.345 | 63.375 | 36.629 | 1.00 | 65.40 |
| 2103 | CG2 | THR | B | 398 | 37.471 | 65.016 | 37.992 | 1.00 | 63.39 |
| 2104 | N | VAL | B | 399 | 40.241 | 63.817 | 38.729 | 1.00 | 51.84 |
| 2105 | CA | VAL | B | 399 | 41.163 | 64.279 | 39.751 | 1.00 | 48.05 |
| 2106 | C | VAL | B | 399 | 41.515 | 65.730 | 39.490 | 1.00 | 44.81 |
| 2107 | O | VAL | B | 399 | 41.911 | 66.098 | 38.381 | 1.00 | 44.41 |
| 2108 | CB | VAL | B | 399 | 42.466 | 63.468 | 39.751 | 1.00 | 49.35 |
| 2109 | CG1 | VAL | B | 399 | 43.409 | 64.010 | 40.805 | 1.00 | 49.43 |
| 2110 | CG2 | VAL | B | 399 | 42.168 | 62.006 | 40.010 | 1.00 | 53.10 |
| 2111 | N | THR | B | 400 | 41.354 | 66.564 | 40.504 | 1.00 | 42.44 |
| 2112 | CA | THR | B | 400 | 41.691 | 67.965 | 40.343 | 1.00 | 40.44 |
| 2113 | C | THR | B | 400 | 42.532 | 68.449 | 41.498 | 1.00 | 38.07 |
| 2114 | O | THR | B | 400 | 42.483 | 67.908 | 42.602 | 1.00 | 36.13 |
| 2115 | CB | THR | B | 400 | 40.448 | 68.869 | 40.266 | 1.00 | 39.64 |
| 2116 | OG1 | THR | B | 400 | 39.845 | 68.962 | 41.561 | 1.00 | 43.72 |
| 2117 | CG2 | THR | B | 400 | 39.442 | 68.322 | 39.278 | 1.00 | 38.32 |
| 2118 | N | SER | B | 401 | 43.335 | 69.460 | 41.216 | 1.00 | 36.90 |
| 2119 | CA | SER | B | 401 | 44.168 | 70.069 | 42.224 | 1.00 | 35.46 |
| 2120 | C | SER | B | 401 | 43.988 | 71.557 | 42.016 | 1.00 | 33.95 |
| 2121 | O | SER | B | 401 | 44.153 | 72.062 | 40.908 | 1.00 | 34.80 |
| 2122 | CB | SER | B | 401 | 45.638 | 69.694 | 42.040 | 1.00 | 36.91 |
| 2123 | OG | SER | B | 401 | 46.417 | 70.301 | 43.060 | 1.00 | 40.84 |
| 2124 | N | THR | B | 402 | 43.635 | 72.248 | 43.086 | 1.00 | 32.28 |
| 2125 | CA | THR | B | 402 | 43.422 | 73.672 | 43.032 | 1.00 | 31.85 |
| 2126 | C | THR | B | 402 | 44.508 | 74.355 | 43.836 | 1.00 | 31.76 |
| 2127 | O | THR | B | 402 | 44.737 | 74.046 | 45.010 | 1.00 | 30.55 |
| 2128 | CB | THR | B | 402 | 42.051 | 74.030 | 43.595 | 1.00 | 31.33 |
| 2129 | OG1 | THR | B | 402 | 41.058 | 73.316 | 42.862 | 1.00 | 32.83 |
| 2130 | CG2 | THR | B | 402 | 41.782 | 75.522 | 43.468 | 1.00 | 32.70 |
| 2131 | N | LEU | B | 403 | 45.167 | 75.301 | 43.188 | 1.00 | 30.72 |
| 2132 | CA | LEU | B | 403 | 46.257 | 76.018 | 43.800 | 1.00 | 30.33 |
| 2133 | C | LEU | B | 403 | 45.927 | 77.485 | 44.003 | 1.00 | 29.66 |
| 2134 | O | LEU | B | 403 | 45.608 | 78.185 | 43.048 | 1.00 | 28.06 |
| 2135 | CB | LEU | B | 403 | 47.502 | 75.902 | 42.913 | 1.00 | 28.34 |
| 2136 | CG | LEU | B | 403 | 48.769 | 76.632 | 43.381 | 1.00 | 32.10 |
| 2137 | CD1 | LEU | B | 403 | 49.425 | 75.823 | 44.503 | 1.00 | 31.94 |
| 2138 | CD2 | LEU | B | 403 | 49.756 | 76.790 | 42.224 | 1.00 | 30.42 |
| 2139 | N | PRO | B | 404 | 45.983 | 77.962 | 45.259 | 1.00 | 31.48 |
| 2140 | CA | PRO | B | 404 | 45.704 | 79.367 | 45.566 | 1.00 | 33.20 |
| 2141 | C | PRO | B | 404 | 46.841 | 80.149 | 44.906 | 1.00 | 34.02 |
| 2142 | O | PRO | B | 404 | 47.999 | 79.755 | 44.996 | 1.00 | 32.25 |
| 2143 | CB | PRO | B | 404 | 45.788 | 79.417 | 47.090 | 1.00 | 33.28 |
| 2144 | CG | PRO | B | 404 | 45.435 | 78.010 | 47.500 | 1.00 | 31.82 |
| 2145 | CD | PRO | B | 404 | 46.186 | 77.187 | 46.497 | 1.00 | 31.26 |
| 2146 | N | VAL | B | 405 | 46.503 | 81.238 | 44.235 | 1.00 | 34.94 |
| 2147 | CA | VAL | B | 405 | 47.483 | 82.044 | 43.536 | 1.00 | 38.63 |
| 2148 | C | VAL | B | 405 | 47.541 | 83.472 | 44.068 | 1.00 | 40.02 |
| 2149 | O | VAL | B | 405 | 46.537 | 84.027 | 44.502 | 1.00 | 40.31 |
| 2150 | CB | VAL | B | 405 | 47.143 | 82.052 | 42.018 | 1.00 | 42.09 |
| 2151 | CD1 | VAL | B | 405 | 47.110 | 83.472 | 41.468 | 1.00 | 43.95 |
| 2152 | CG2 | VAL | B | 405 | 48.144 | 81.195 | 41.273 | 1.00 | 41.76 |
| 2153 | N | GLY | B | 406 | 48.728 | 84.064 | 44.039 | 1.00 | 42.35 |
| 2154 | CA | GLY | B | 406 | 48.859 | 85.432 | 44.503 | 1.00 | 43.35 |
| 2155 | C | GLY | B | 406 | 48.178 | 86.354 | 43.513 | 1.00 | 43.87 |
| 2156 | O | GLY | B | 406 | 48.286 | 86.154 | 42.303 | 1.00 | 42.68 |
| 2157 | N | THR | B | 407 | 47.466 | 87.352 | 44.023 | 1.00 | 44.40 |
| 2158 | CA | THR | B | 407 | 46.760 | 88.306 | 43.177 | 1.00 | 47.37 |
| 2159 | C | THR | B | 407 | 47.713 | 89.127 | 42.322 | 1.00 | 48.27 |
| 2160 | O | TNR | B | 407 | 47.549 | 89.208 | 41.107 | 1.00 | 48.77 |
| 2161 | CB | THR | B | 407 | 45.922 | 89.288 | 44.022 | 1.00 | 48.44 |
| 2262 | OG1 | THR | B | 407 | 44.809 | 88.599 | 44.596 | 1.00 | 50.00 |
| 2163 | CG2 | THR | B | 407 | 45.413 | 90.436 | 43.162 | 1.00 | 53.23 |
| 2164 | N | ARG | B | 408 | 48.697 | 89.746 | 42.970 | 1.00 | 48.10 |
| 2165 | CA | ARG | B | 408 | 49.665 | 90.579 | 42.279 | 1.00 | 48.54 |
| 2166 | C | ARG | B | 408 | 50.540 | 89.773 | 41.335 | 1.00 | 48.48 |
| 2167 | O | ARG | B | 408 | 50.769 | 90.182 | 40.197 | 1.00 | 47.34 |
| 2168 | CB | ARG | B | 408 | 50.530 | 91.325 | 43.290 | 1.00 | 50.16 |
| 2169 | N | ASP | B | 409 | 51.033 | 88.634 | 41.806 | 1.00 | 48.72 |
| 2170 | CA | ASP | B | 409 | 51.886 | 87.787 | 40.979 | 1.00 | 50.88 |
| 2171 | C | ASP | B | 409 | 51.187 | 87.421 | 39.674 | 1.00 | 49.92 |
| 2172 | O | ASP | B | 409 | 51.789 | 87.468 | 38.598 | 1.00 | 49.76 |
| 2173 | CB | ASP | B | 409 | 52.260 | 86.512 | 41.738 | 1.00 | 56.18 |
| 2174 | CG | ASP | B | 409 | 53.013 | 86.801 | 43.026 | 1.00 | 64.80 |
| 2175 | OD1 | ASP | B | 409 | 54.062 | 87.482 | 42.956 | 1.00 | 71.41 |
| 2176 | OD2 | ASP | B | 409 | 52.561 | 86.353 | 44.105 | 1.00 | 68.29 |
| 2177 | N | TRP | B | 410 | 49.908 | 87.067 | 39.773 | 1.00 | 48.36 |
| 2178 | CA | TRP | B | 410 | 49.142 | 86.686 | 38.597 | 1.00 | 46.32 |
| 2179 | C | TRP | B | 410 | 48.953 | 87.864 | 37.653 | 1.00 | 46.21 |
| 2180 | O | TRP | B | 410 | 49.111 | 87.731 | 36.440 | 1.00 | 44.64 |
| 2181 | CB | TRP | B | 410 | 47.774 | 86.120 | 38.992 | 1.00 | 42.82 |
| 2182 | CG | TRP | B | 410 | 47.001 | 85.710 | 37.796 | 1.00 | 39.31 |
| 2183 | CD1 | TRP | B | 410 | 46.104 | 86.467 | 37.103 | 1.00 | 37.29 |
| 2184 | CD2 | TRP | B | 410 | 47.181 | 84.507 | 37.043 | 1.00 | 35.49 |
| 2185 | NE1 | TRP | B | 410 | 45.726 | 85.819 | 35.957 | 1.00 | 39.24 |
| 2186 | CE2 | TRP | B | 410 | 46.372 | 84.613 | 35.895 | 1.00 | 32.76 |
| 2187 | CE3 | TRP | B | 410 | 47.956 | 83.351 | 37.228 | 1.00 | 32.07 |
| 2188 | CZ2 | TRP | B | 410 | 46.309 | 83.606 | 34.924 | 1.00 | 29.59 |
| 2189 | CZ3 | TRP | B | 410 | 47.896 | 82.352 | 36.269 | 1.00 | 33.68 |
| 2190 | CH2 | TRP | B | 410 | 47.073 | 82.489 | 35.125 | 1.00 | 30.56 |
| 2191 | N | ILE | B | 411 | 48.611 | 89.016 | 38.212 | 1.00 | 48.11 |
| 2192 | CA | ILE | B | 411 | 48.408 | 90.209 | 37.401 | 1.00 | 50.90 |
| 2193 | C | ILE | B | 411 | 49.712 | 90.649 | 36.742 | 1.00 | 52.46 |
| 2194 | O | ILE | B | 411 | 49.698 | 91.229 | 35.658 | 1.00 | 53.34 |
| 2195 | CB | ILE | B | 411 | 47.845 | 91.366 | 38.246 | 1.00 | 51.58 |
| 2196 | CG1 | ILE | B | 411 | 46.456 | 90.987 | 38.770 | 1.00 | 52.81 |
| 2197 | CG2 | ILE | B | 411 | 47.772 | 92.634 | 37.415 | 1.00 | 51.95 |
| 2198 | CD1 | ILE | B | 411 | 45.823 | 92.034 | 39.669 | 1.00 | 59.38 |
| 2199 | N | GLU | B | 412 | 50.837 | 90.348 | 37.385 | 1.00 | 53.21 |
| 2200 | CA | GLU | B | 412 | 52.137 | 90.728 | 36.852 | 1.00 | 53.12 |
| 2201 | C | GLU | B | 412 | 52.681 | 89.747 | 35.831 | 1.00 | 51.24 |
| 2202 | O | GLU | B | 412 | 53.736 | 89.990 | 35.252 | 1.00 | 51.40 |
| 2203 | CB | GLU | B | 412 | 53.152 | 90.909 | 37.981 | 1.00 | 59.71 |
| 2204 | CG | GLU | B | 412 | 53.022 | 92.238 | 38.717 | 1.00 | 70.52 |
| 2205 | CD | GLU | B | 412 | 54.060 | 92.399 | 39.810 | 1.00 | 78.58 |
| 2206 | OE1 | GLU | B | 412 | 55.262 | 92.198 | 39.527 | 1.00 | 83.44 |
| 2207 | OE2 | GLU | B | 412 | 53.676 | 92.732 | 40.950 | 1.00 | 82.10 |
| 2208 | N | GLY | B | 413 | 51.982 | 88.634 | 35.623 | 1.00 | 48.47 |
| 2209 | CA | GLY | B | 413 | 52.427 | 87.677 | 34.626 | 1.00 | 44.62 |
| 2210 | C | GLY | B | 413 | 53.116 | 86.387 | 35.032 | 1.00 | 44.15 |
| 2211 | O | GLY | B | 413 | 53.687 | 85.718 | 34.173 | 1.00 | 43.50 |
| 2212 | N | GLU | B | 414 | 53.085 | 86.012 | 36.307 | 1.00 | 42.78 |
| 2213 | CA | GLU | B | 414 | 53.722 | 84.757 | 36.694 | 1.00 | 40.78 |
| 2214 | C | GLU | B | 414 | 53.105 | 83.652 | 35.851 | 1.00 | 39.97 |
| 2215 | O | GLU | B | 414 | 51.955 | 83.758 | 35.410 | 1.00 | 37.43 |
| 2216 | CB | GLU | B | 414 | S3.489 | 84.445 | 38.182 | 1.00 | 41.96 |
| 2217 | CG | GLU | B | 414 | S4.034 | 83.081 | 38.659 | 1.00 | 44.09 |
| 2218 | CD | GLU | B | 414 | 55.564 | 83.018 | 38.740 | 1.00 | 49.01 |
| 2219 | OE1 | GLU | B | 414 | 56.213 | 82.549 | 37.775 | 1.00 | 46.84 |
| 2220 | OE2 | GLU | B | 414 | 56.121 | 83.448 | 39.773 | 1.00 | 49.89 |
| 2221 | N | THR | B | 415 | 53.879 | 82.599 | 35.615 | 1.00 | 39.56 |
| 2222 | CA | THR | B | 415 | 53.403 | 81.464 | 34.847 | 1.00 | 40.76 |
| 2223 | C | THR | B | 415 | 53.487 | 80.231 | 35.738 | 1.00 | 40.07 |
| 2224 | O | THR | B | 415 | 54.533 | 79.960 | 36.323 | 1.00 | 42.10 |
| 2225 | CB | THR | B | 415 | 54.238 | 81.273 | 33.556 | 1.00 | 44.69 |
| 2226 | OG1 | THR | B | 415 | 54.222 | 79.893 | 33.171 | 1.00 | 51.98 |
| 2227 | CG2 | THR | B | 415 | 55.652 | 81.744 | 33.758 | 1.00 | 50.00 |
| 2228 | N | TYR | B | 416 | 52.380 | 79.500 | 35.860 | 1.00 | 37.57 |
| 2229 | CA | TYR | B | 416 | 52.327 | 78.318 | 36.714 | 1.00 | 34.50 |
| 2230 | C | TYR | B | 416 | 52.265 | 77.051 | 35.899 | 1.00 | 36.77 |
| 2231 | O | TYR | B | 416 | 51.706 | 77.038 | 34.800 | 1.00 | 35.94 |
| 2232 | CB | TYR | B | 416 | 51.116 | 78.379 | 37.634 | 1.00 | 34.15 |
| 2233 | CG | TYR | B | 416 | 51.085 | 79.597 | 38.514 | 1.00 | 30.65 |
| 2234 | CD1 | TYR | B | 416 | 50.777 | 80.856 | 37.992 | 1.00 | 32.53 |
| 2235 | CD2 | TYR | B | 416 | 51.389 | 79.499 | 39.865 | 1.00 | 31.72 |
| 2236 | CE1 | TYR | B | 416 | 50.772 | 81.984 | 38.803 | 1.00 | 34.27 |
| 2237 | CE2 | TYR | B | 416 | 51.390 | 80.619 | 40.680 | 1.00 | 32.11 |
| 2238 | CZ | TYR | B | 416 | 51.080 | 81.856 | 40.144 | 1.00 | 33.86 |
| 2239 | OH | TYR | B | 416 | 51.082 | 82.964 | 40.961 | 1.00 | 41.22 |
| 2240 | N | GLN | B | 417 | 52.829 | 75.976 | 36.441 | 1.00 | 37.58 |
| 2241 | CA | GLN | B | 417 | 52.848 | 74.722 | 35.714 | 1.00 | 39.43 |
| 2242 | C | GLN | B | 417 | 52.341 | 73.538 | 36.518 | 1.00 | 39.40 |
| 2243 | O | GLN | B | 417 | 52.664 | 73.373 | 37.694 | 1.00 | 39.63 |
| 2244 | CB | GLN | B | 417 | 54.269 | 74.421 | 35.211 | 1.00 | 41.37 |
| 2245 | CG | GLN | B | 417 | 54.350 | 73.214 | 34.278 | 1.00 | 51.81 |
| 2246 | CD | GLN | B | 417 | 55.773 | 72.856 | 33.886 | 1.00 | 61.69 |
| 2247 | CE1 | GLN | B | 417 | 56.555 | 72.371 | 34.710 | 1.00 | 67.15 |
| 2248 | NE2 | GLN | B | 417 | 56.119 | 73.096 | 32.621 | 1.00 | 63.64 |
| 2249 | N | CYS | B | 418 | 51.543 | 72.712 | 35.864 | 1.00 | 39.29 |
| 2250 | CA | CYS | B | 418 | 51.019 | 71.517 | 36.489 | 1.00 | 41.80 |
| 2251 | C | CYS | B | 418 | 51.720 | 70.350 | 35.818 | 1.00 | 42.50 |
| 2252 | O | CYS | B | 418 | 51.646 | 70.191 | 34.600 | 1.00 | 43.33 |
| 2253 | CB | CYS | B | 418 | 49.501 | 71.392 | 36.275 | 1.00 | 41.95 |
| 2254 | SC | CYS | B | 418 | 48.795 | 69.904 | 37.057 | 1.00 | 54.15 |
| 2255 | N | ARG | B | 419 | 52.418 | 69.551 | 36.609 | 1.00 | 43.98 |
| 2256 | CA | ARG | B | 419 | 53.103 | 68.375 | 36.093 | 1.00 | 47.51 |
| 2257 | C | ARG | B | 419 | 52.277 | 67.172 | 36.542 | 1.00 | 48.11 |
| 2258 | O | ARG | B | 419 | 52.219 | 66.859 | 37.730 | 1.00 | 47.85 |
| 2259 | CB | ARG | B | 419 | 54.532 | 68.311 | 36.648 | 1.00 | 49.08 |
| 2260 | CG | ARG | B | 419 | 55.203 | 66.958 | 36.514 | 1.00 | 54.11 |
| 2261 | CD | ARG | B | 419 | 56.708 | 67.046 | 36.796 | 1.00 | 64.71 |
| 2262 | NE | ARG | B | 419 | 57.325 | 65.732 | 36.963 | 1.00 | 64.09 |
| 2263 | CZ | ARG | B | 419 | 57.437 | 65.107 | 38.132 | 1.00 | 70.31 |
| 2264 | NE1 | ARG | B | 419 | 56.981 | 65.681 | 39.239 | 1.00 | 68.32 |
| 2265 | NH2 | ARG | B | 419 | 57.992 | 63.901 | 38.195 | 1.00 | 71.53 |
| 2266 | N | VAL | B | 420 | 51.610 | 66.524 | 35.592 | 1.00 | 51.04 |
| 2267 | CA | VAL | B | 420 | 50.776 | 65.369 | 35.900 | 1.00 | 56.22 |
| 2268 | C | VAL | B | 420 | 51.561 | 64.068 | 35.746 | 1.00 | 61.25 |
| 2269 | O | VAL | B | 420 | 52.178 | 63.820 | 34.707 | 1.00 | 60.11 |
| 2270 | CB | VAL | B | 420 | 49.536 | 65.305 | 34.985 | 1.00 | 54.44 |
| 2271 | CG1 | VAL | B | 420 | 48.678 | 64.106 | 35.371 | 1.00 | 52.69 |
| 2272 | CG2 | VAL | B | 420 | 48.732 | 66.597 | 35.095 | 1.00 | 55.45 |
| 2273 | N | THR | B | 421 | 51.529 | 63.241 | 36.786 | 1.00 | 66.23 |
| 2274 | CA | THR | B | 421 | 52.244 | 61.969 | 36.774 | 1.00 | 73.18 |
| 2275 | C | THR | B | 421 | 51.334 | 60.766 | 36.970 | 1.00 | 76.87 |
| 2276 | O | THR | B | 421 | 50.968 | 60.443 | 38.100 | 1.00 | 77.93 |
| 2277 | CB | THR | B | 421 | 53.317 | 61.929 | 37.875 | 1.00 | 73.75 |
| 2278 | OG1 | THR | B | 421 | 54.272 | 62.972 | 37.649 | 1.00 | 77.71 |
| 2279 | CG2 | THR | B | 421 | 54.033 | 60.591 | 37.873 | 1.00 | 75.35 |
| 2280 | N | HIS | B | 422 | 50.976 | 60.101 | 35.874 | 1.00 | 81.02 |
| 2281 | CA | HIS | B | 422 | 50.117 | 58.922 | 35.949 | 1.00 | 86.07 |
| 2282 | C | HIS | B | 422 | 51.004 | 57.686 | 35.822 | 1.00 | 89.67 |
| 2283 | O | HIS | B | 422 | 51.920 | 57.653 | 35.002 | 1.00 | 90.00 |
| 2284 | CB | HIS | B | 422 | 49.071 | 58.940 | 34.831 | 1.00 | 87.43 |
| 2285 | CG | HIS | B | 422 | 47.892 | 58.052 | 35.092 | 1.00 | 90.73 |
| 2286 | ND1 | HIS | B | 422 | 47.076 | 58.203 | 36.191 | 1.00 | 93.83 |
| 2287 | CD2 | HIS | B | 422 | 47.396 | 57.000 | 34.396 | 1.00 | 91.89 |
| 2288 | CE1 | HIS | B | 422 | 46.127 | 57.282 | 36.164 | 1.00 | 94.70 |
| 2289 | NE2 | HIS | B | 422 | 46.300 | 56.540 | 35.085 | 1.00 | 93.25 |
| 2290 | N | PRO | B | 423 | 50.729 | 56.646 | 36.626 | 1.00 | 92.64 |
| 2291 | CA | PRO | B | 423 | 51.489 | 55.391 | 36.640 | 1.00 | 95.19 |
| 2292 | C | PRO | B | 423 | 51.972 | 54.762 | 35.325 | 1.00 | 97.06 |
| 2293 | O | PRO | B | 423 | 53.169 | 54.536 | 35.155 | 1.00 | 97.57 |
| 2294 | CB | PRO | B | 423 | 50.576 | 54.445 | 37.433 | 1.00 | 95.56 |
| 2295 | CG | PRO | B | 423 | 49.209 | 55.034 | 37.247 | 1.00 | 93.94 |
| 2296 | CD | PRO | B | 423 | 49.488 | 56.495 | 37.404 | 1.00 | 93.12 |
| 2297 | N | HIS | B | 424 | 51.058 | 54.495 | 34.399 | 1.00 | 98.71 |
| 2298 | CA | HIS | B | 424 | 51.410 | 53.831 | 33.143 | 1.00 | 100.16 |
| 2299 | C | HIS | B | 424 | 52.104 | 54.579 | 32.005 | 1.00 | 99.67 |
| 2300 | O | HIS | B | 424 | 52.910 | 53.991 | 31.284 | 1.00 | 99.61 |
| 2301 | CB | HIS | B | 424 | 50.165 | 53.149 | 32.578 | 1.00 | 102.42 |
| 2302 | CG | HIS | B | 424 | 49.578 | 52.125 | 33.495 | 1.00 | 105.47 |
| 2303 | ND1 | HIS | B | 424 | 48.302 | 52.228 | 34.008 | 1.00 | 107.10 |
| 2304 | CD2 | HIS | B | 424 | 50.079 | 50.961 | 33.967 | 1.00 | 97.46 |
| 2305 | CE1 | HIS | B | 424 | 48.044 | 51.170 | 34.755 | 1.00 | 106.81 |
| 2306 | NE2 | HIS | B | 424 | 49.106 | 50.385 | 34.747 | 1.00 | 106.61 |
| 2307 | N | LEU | B | 425 | 51.802 | 55.858 | 31.833 | 1.00 | 99.19 |
| 2308 | CA | LEU | B | 425 | 52.384 | 56.628 | 30.737 | 1.00 | 99.01 |
| 2309 | C | LEU | B | 425 | 53.886 | 56.908 | 30.816 | 1.00 | 98.36 |
| 2310 | O | LEU | B | 425 | 54.440 | 57.121 | 31.896 | 1.00 | 98.39 |
| 2311 | CB | LEU | B | 425 | 51.606 | 57.929 | 30.593 | 1.00 | 100.66 |
| 2312 | CG | LEU | B | 425 | 50.105 | 57.631 | 30.633 | 1.00 | 101.95 |
| 2313 | CD1 | LEU | B | 425 | 49.315 | 58.917 | 30.531 | 1.00 | 102.59 |
| 2314 | CD2 | LEU | B | 425 | 49.744 | 56.677 | 29.502 | 1.00 | 103.85 |
| 2315 | N | PRO | B | 426 | 54.562 | 56.915 | 29.652 | 1.00 | 97.57 |
| 2316 | CA | PRO | B | 426 | 56.004 | 57.165 | 29.560 | 1.00 | 96.82 |
| 2317 | C | PRO | B | 426 | 56.414 | 58.525 | 30.108 | 1.00 | 95.53 |
| 2318 | O | PRO | B | 426 | 57.040 | 58.610 | 31.163 | 1.00 | 95.77 |
| 2319 | CB | PRO | B | 426 | 56.282 | 57.030 | 28.063 | 1.00 | 97.13 |
| 2320 | CG | PRO | B | 426 | 54.995 | 57.494 | 27.440 | 1.00 | 97.88 |
| 2321 | CD | PRO | B | 426 | 53.969 | 56.798 | 28.307 | 1.00 | 97.70 |
| 2322 | N | ARG | B | 427 | 56.064 | 59.585 | 29.387 | 1.00 | 94.16 |
| 2323 | CA | ARG | B | 427 | 56.402 | 60.931 | 29.826 | 1.00 | 92.52 |
| 2324 | C | ARG | B | 427 | 55.285 | 61.509 | 30.692 | 1.00 | 89.85 |
| 2325 | O | ARG | B | 427 | 54.123 | 61.113 | 30.583 | 1.00 | 89.26 |
| 2326 | CB | ARG | B | 427 | 56.652 | 61.851 | 28.622 | 1.00 | 97.34 |
| 2327 | CG | ARG | B | 427 | 57.202 | 63.219 | 29.023 | 1.00 | 99.32 |
| 2328 | CD | ARG | B | 427 | 57.407 | 64.172 | 27.847 | 1.00 | 105.99 |
| 2329 | NE | ARG | B | 427 | 58.068 | 65.403 | 28.285 | 1.00 | 111.10 |
| 2330 | CZ | ARG | B | 427 | 58.302 | 66.460 | 27.512 | 1.00 | 111.66 |
| 2331 | NH1 | ARG | B | 427 | 57.927 | 66.457 | 26.239 | 1.00 | 111.70 |
| 2332 | NH2 | ARG | B | 427 | 58.921 | 67.522 | 28.013 | 1.00 | 110.09 |
| 2333 | N | ALA | B | 428 | 55.651 | 62.442 | 31.562 | 1.00 | 86.56 |
| 2334 | CA | ALA | B | 428 | 54.690 | 63.087 | 32.439 | 1.00 | 82.70 |
| 2335 | C | ALA | B | 428 | 53.998 | 64.211 | 31.677 | 1.00 | 80.03 |
| 2336 | O | ALA | B | 428 | 54.619 | 64.889 | 30.861 | 1.00 | 80.29 |
| 2337 | CB | ALA | B | 428 | 55.400 | 63.647 | 33.663 | 1.00 | 83.85 |
| 2338 | N | LEU | B | 429 | 52.708 | 64.397 | 31.936 | 1.00 | 76.76 |
| 2339 | CA | LEU | B | 429 | 51.949 | 65.456 | 31.285 | 1.00 | 72.72 |
| 2340 | C | LEU | B | 429 | 52.311 | 66.780 | 31.936 | 1.00 | 69.67 |
| 2341 | O | LEU | B | 429 | 52.472 | 66.858 | 33.153 | 1.00 | 68.64 |
| 2342 | CB | LEU | B | 429 | 50.444 | 65.208 | 31.429 | 1.00 | 73.60 |
| 2343 | CG | LEU | B | 429 | 49.753 | 64.431 | 30.307 | 1.00 | 73.35 |
| 2344 | CD1 | LEU | B | 429 | 48.320 | 64.102 | 30.696 | 1.00 | 72.09 |
| 2345 | CD2 | LEU | B | 429 | 49.787 | 65.267 | 29.035 | 1.00 | 76.40 |
| 2346 | N | MET | B | 430 | 52.447 | 67.821 | 31.124 | 1.00 | 66.83 |
| 2347 | CA | MET | B | 430 | 52.786 | 69.132 | 31.651 | 1.00 | 64.81 |
| 2348 | C | MET | B | 430 | 51.922 | 70.216 | 31.026 | 1.00 | 61.41 |
| 2349 | O | MET | B | 430 | 51.824 | 70.326 | 29.801 | 1.00 | 60.84 |
| 2350 | CB | MET | B | 430 | 54.265 | 69.427 | 31.409 | 1.00 | 68.22 |
| 2351 | CG | MET | B | 430 | 55.184 | 68.353 | 31.967 | 1.00 | 74.39 |
| 2352 | SD | MET | B | 430 | 56.921 | 68.714 | 31.713 | 1.00 | 83.18 |
| 2353 | CE | MET | B | 430 | 56.993 | 68.764 | 29.906 | 1.00 | 84.12 |
| 2354 | N | ARG | B | 431 | 51.288 | 71.010 | 31.882 | 1.00 | 57.17 |
| 2355 | CA | ARG | B | 431 | 50.428 | 72.094 | 31.426 | 1.00 | 54.04 |
| 2356 | C | ARG | B | 431 | 50.848 | 73.366 | 32.137 | 1.00 | 50.58 |
| 2357 | O | ARG | B | 431 | 51.233 | 73.330 | 33.304 | 1.00 | 50.46 |
| 2358 | CB | ARG | B | 431 | 48.959 | 71.775 | 31.736 | 1.00 | 55.16 |
| 2359 | CG | ARG | B | 431 | 48.413 | 70.564 | 30.991 | 1.00 | 59.24 |
| 2360 | CD | ARG | B | 431 | 48.428 | 70.798 | 29.489 | 1.00 | 63.93 |
| 2361 | NE | ARG | B | 431 | 48.014 | 69.617 | 28.737 | 1.00 | 70.30 |
| 2362 | CZ | ARG | B | 431 | 48.043 | 69.530 | 27.411 | 1.00 | 76.24 |
| 2363 | NH1 | ARG | B | 431 | 48.469 | 70.559 | 26.683 | 1.00 | 77.83 |
| 2364 | NH2 | ARG | B | 431 | 47.648 | 68.415 | 26.811 | 1.00 | 77.12 |
| 2365 | N | SER | B | 432 | 50.788 | 74.488 | 31.431 | 1.00 | 46.92 |
| 2366 | CA | SER | B | 432 | 51.163 | 75.765 | 32.016 | 1.00 | 45.15 |
| 2367 | C | SER | B | 432 | 50.118 | 76.816 | 31.702 | 1.00 | 42.49 |
| 2368 | O | SER | B | 432 | 49.382 | 76.699 | 30.721 | 1.00 | 42.76 |
| 2369 | CB | SER | B | 432 | 52.532 | 76.206 | 31.490 | 1.00 | 44.31 |
| 2370 | OG | SER | B | 432 | 52.516 | 76.284 | 30.082 | 1.00 | 49.96 |
| 2371 | N | THR | B | 433 | 50.060 | 77.851 | 32.530 | 1.00 | 40.28 |
| 2372 | CA | THR | B | 433 | 49.078 | 78.909 | 32.337 | 1.00 | 39.71 |
| 2373 | C | THR | B | 433 | 49.604 | 80.244 | 32.843 | 1.00 | 39.49 |
| 2374 | O | THR | B | 433 | 50.414 | 80.285 | 33.766 | 1.00 | 40.22 |
| 2375 | CB | THR | B | 433 | 47.761 | 78.577 | 33.078 | 1.00 | 38.07 |
| 2376 | OG1 | THR | B | 433 | 46.793 | 79.594 | 32.805 | 1.00 | 39.56 |
| 2377 | CG2 | THR | B | 433 | 47.991 | 78.512 | 34.582 | 1.00 | 36.99 |
| 2378 | N | THR | B | 434 | 49.139 | 81.328 | 32.226 | 1.00 | 39.18 |
| 2379 | CA | THR | B | 434 | 49.532 | 82.687 | 32.588 | 1.00 | 40.70 |
| 2380 | C | THR | B | 434 | 48.414 | 83.635 | 32.166 | 1.00 | 40.23 |
| 2381 | O | THR | B | 434 | 47.509 | 83.236 | 31.441 | 1.00 | 41.18 |
| 2382 | CB | THR | B | 434 | 50.816 | 83.116 | 31.844 | 1.00 | 43.01 |
| 2383 | OG1 | THR | B | 434 | 50.548 | 83.238 | 30.439 | 1.00 | 47.85 |
| 2384 | CG2 | THR | B | 434 | 51.874 | 82.076 | 31.995 | 1.00 | 48.67 |
| 2385 | N | LYS | B | 435 | 48.475 | 84.886 | 32.604 | 1.00 | 40.97 |
| 2386 | CA | LYS | B | 435 | 47.450 | 85.833 | 32.211 | 1.00 | 45.73 |
| 2387 | C | LYS | B | 435 | 47.579 | 85.999 | 30.702 | 1.00 | 48.93 |
| 2388 | O | LYS | B | 435 | 48.670 | 85.890 | 30.158 | 1.00 | 48.49 |
| 2389 | CB | LYS | B | 435 | 47.663 | 87.166 | 32.906 | 1.00 | 45.94 |
| 2390 | N | THR | B | 436 | 46.467 | 86.235 | 30.018 | 1.00 | 52.01 |
| 2391 | CA | THR | B | 436 | 46.520 | 86.429 | 28.574 | 1.00 | 54.88 |
| 2392 | C | THR | B | 436 | 46.916 | 87.878 | 28.326 | 1.00 | 55.70 |
| 2393 | O | THR | B | 436 | 46.454 | 88.777 | 29.023 | 1.00 | 55.21 |
| 2394 | CB | THR | B | 436 | 45.146 | 86.162 | 27.909 | 1.00 | 56.24 |
| 2395 | OG1 | THR | B | 436 | 44.798 | 84.779 | 28.062 | 1.00 | 60.56 |
| 2396 | CG2 | THR | B | 436 | 45.188 | 86.510 | 26.423 | 1.00 | 57.04 |
| 2397 | N | SER | B | 437 | 47.790 | 88.099 | 27.352 | 1.00 | 57.93 |
| 2398 | CA | SER | B | 437 | 48.220 | 89.451 | 27.020 | 1.00 | 60.42 |
| 2399 | C | SER | B | 437 | 47.361 | 89.917 | 25.851 | 1.00 | 60.49 |
| 2400 | O | SER | B | 437 | 46.397 | 89.243 | 25.477 | 1.00 | 60.21 |
| 2401 | CB | SER | B | 437 | 49.706 | 89.467 | 26.630 | 1.00 | 61.57 |
| 2402 | OC | SER | B | 437 | 49.949 | 88.643 | 25.500 | 1.00 | 65.51 |
| 2403 | N | GLY | B | 438 | 47.705 | 91.067 | 25.279 | 1.00 | 60.09 |
| 2404 | CA | GLY | B | 438 | 46.934 | 91.578 | 24.161 | 1.00 | 58.42 |
| 2405 | C | GLY | B | 438 | 45.970 | 92.663 | 24.589 | 1.00 | 57.80 |
| 2406 | O | GLY | B | 438 | 45.853 | 92.952 | 25.778 | 1.00 | 56.51 |
| 2407 | N | PRO | B | 439 | 45.267 | 93.291 | 23.633 | 1.00 | 57.81 |
| 2408 | CA | PRO | B | 439 | 44.305 | 94.358 | 23.915 | 1.00 | 56.19 |
| 2409 | C | PRO | B | 439 | 43.178 | 93.908 | 24.837 | 1.00 | 53.91 |
| 2410 | O | PRO | B | 439 | 42.891 | 92.714 | 24.953 | 1.00 | 53.91 |
| 2411 | CB | PRO | B | 439 | 43.794 | 94.735 | 22.524 | 1.00 | 57.79 |
| 2412 | CG | PRO | B | 439 | 44.967 | 94.453 | 21.652 | 1.00 | 58.60 |
| 2413 | CD | PRO | B | 439 | 45.427 | 93.115 | 22.179 | 1.00 | 60.65 |
| 2414 | N | ARG | B | 440 | 42.545 | 94.878 | 25.488 | 1.00 | 51.09 |
| 2415 | CA | ARG | B | 440 | 41.437 | 94.610 | 26.398 | 1.00 | 48.90 |
| 2416 | C | ARG | B | 440 | 40.235 | 95.380 | 25.869 | 1.00 | 45.83 |
| 2417 | O | ARG | B | 440 | 40.398 | 96.412 | 25.219 | 1.00 | 45.88 |
| 2418 | CB | ARG | B | 440 | 41.764 | 95.099 | 27.811 | 1.00 | 49.95 |
| 2419 | CG | ARG | B | 440 | 43.127 | 94.681 | 28.329 | 1.00 | 59.03 |
| 2420 | CD | ARG | B | 440 | 43.227 | 93.194 | 28.652 | 1.00 | 65.31 |
| 2421 | NE | ARG | B | 440 | 44.627 | 92.786 | 28.758 | 1.00 | 72.31 |
| 2422 | CZ | ARG | B | 440 | 45.047 | 91.614 | 29.222 | 1.00 | 74.99 |
| 2423 | NH1 | ARG | B | 440 | 44.181 | 90.706 | 29.640 | 1.00 | 77.22 |
| 2424 | NH2 | ARG | B | 440 | 46.345 | 91.348 | 29.263 | 1.00 | 78.59 |
| 2425 | N | ALA | B | 441 | 39.036 | 94.873 | 26.143 | 1.00 | 42.09 |
| 2426 | CA | ALA | B | 441 | 37.800 | 95.521 | 25.713 | 1.00 | 39.26 |
| 2427 | C | ALA | B | 441 | 36.706 | 95.124 | 26.687 | 1.00 | 38.22 |
| 2428 | O | ALA | B | 441 | 36.566 | 93.951 | 27.024 | 1.00 | 38.95 |
| 2429 | CB | ALA | B | 441 | 37.430 | 95.089 | 24.302 | 1.00 | 36.56 |
| 2430 | N | ALA | B | 442 | 35.934 | 96.104 | 27.134 | 1.00 | 37.22 |
| 2431 | CA | ALA | B | 442 | 34.871 | 95.861 | 28.089 | 1.00 | 37.60 |
| 2432 | C | ALA | B | 442 | 33.721 | 95.050 | 27.510 | 1.00 | 38.65 |
| 2433 | O | ALA | B | 442 | 33.497 | 95.047 | 26.302 | 1.00 | 40.22 |
| 2434 | CB | ALA | B | 442 | 34.352 | 97.185 | 28.619 | 1.00 | 37.17 |
| 2435 | N | PRO | B | 443 | 32.981 | 94.340 | 28.378 | 1.00 | 38.04 |
| 2436 | CA | PRO | B | 443 | 31.835 | 93.511 | 27.996 | 1.00 | 36.79 |
| 2437 | C | PRO | B | 443 | 30.581 | 94.376 | 27.857 | 1.00 | 38.14 |
| 2438 | O | PRO | B | 443 | 30.426 | 95.364 | 28.579 | 1.00 | 37.39 |
| 2439 | CB | PRO | B | 443 | 31.677 | 92.550 | 29.179 | 1.00 | 36.30 |
| 2440 | CG | PRO | B | 443 | 32.968 | 92.654 | 29.941 | 1.00 | 36.35 |
| 2441 | CD | PRO | B | 443 | 33.351 | 94.088 | 29.780 | 1.00 | 37.85 |
| 2442 | N | GLU | B | 444 | 29.706 | 94.006 | 26.927 | 1.00 | 36.28 |
| 2443 | CA | GLU | B | 444 | 28.437 | 94.699 | 26.729 | 1.00 | 35.57 |
| 2444 | C | GLU | B | 444 | 27.429 | 93.659 | 27.212 | 1.00 | 33.87 |
| 2445 | O | GLU | B | 444 | 27.495 | 92.490 | 26.809 | 1.00 | 33.43 |
| 2446 | CB | GLU | B | 444 | 28.217 | 95.020 | 25.249 | 1.00 | 38.68 |
| 2447 | N | VAL | B | 445 | 26.503 | 94.080 | 28.067 | 1.00 | 30.18 |
| 2448 | CA | VAL | B | 445 | 25.530 | 93.168 | 28.650 | 1.00 | 28.24 |
| 2449 | C | VAL | B | 445 | 24.077 | 93.464 | 28.289 | 1.00 | 28.44 |
| 2450 | O | VAL | B | 445 | 23.625 | 94.600 | 28.379 | 1.00 | 26.78 |
| 2451 | CB | VAL | B | 445 | 25.663 | 93.183 | 30.195 | 1.00 | 28.10 |
| 2452 | CG1 | VAL | B | 445 | 24.667 | 92.230 | 30.826 | 1.00 | 25.56 |
| 2453 | CG2 | VAL | B | 445 | 27.079 | 92.829 | 30.589 | 1.00 | 26.30 |
| 2454 | N | TYR | B | 446 | 23.349 | 92.425 | 27.897 | 1.00 | 27.87 |
| 2455 | CA | TYR | B | 446 | 21.949 | 92.581 | 27.549 | 1.00 | 27.91 |
| 2456 | C | TYR | B | 446 | 21.140 | 91.407 | 28.076 | 1.00 | 27.06 |
| 2457 | O | TYR | B | 446 | 21.293 | 90.274 | 27.620 | 1.00 | 27.96 |
| 2458 | CB | TYR | B | 446 | 21.764 | 92.702 | 26.026 | 1.00 | 26.45 |
| 2459 | CG | TYR | B | 446 | 20.311 | 92.851 | 25.653 | 1.00 | 26.59 |
| 2460 | CD1 | TYR | B | 446 | 19.543 | 93.886 | 26.195 | 1.00 | 28.48 |
| 2461 | CD2 | TYR | B | 446 | 19.686 | 91.941 | 24.795 | 1.00 | 29.02 |
| 2462 | CE1 | TYR | B | 446 | 18.183 | 94.011 | 25.896 | 1.00 | 33.24 |
| 2463 | CE2 | TYR | B | 446 | 18.326 | 92.057 | 24.484 | 1.00 | 31.33 |
| 2464 | CZ | TYR | B | 446 | 17.585 | 93.094 | 25.041 | 1.00 | 36.01 |
| 2465 | OH | TYR | B | 446 | 16.248 | 93.213 | 24.762 | 1.00 | 36.54 |
| 2466 | N | ALA | B | 447 | 20.274 | 91.686 | 29.040 | 1.00 | 28.07 |
| 2467 | CA | ALA | B | 447 | 19.450 | 90.651 | 29.661 | 1.00 | 27.67 |
| 2468 | C | ALA | B | 447 | 18.021 | 90.785 | 29.168 | 1.00 | 29.19 |
| 2469 | O | ALA | B | 447 | 17.564 | 91.898 | 28.927 | 1.00 | 31.10 |
| 2470 | CB | ALA | B | 447 | 19.502 | 90.798 | 31.169 | 1.00 | 23.97 |
| 2471 | N | PHE | B | 448 | 17.307 | 89.671 | 29.021 | 1.00 | 29.56 |
| 2472 | CA | PHE | B | 448 | 15.943 | 89.751 | 28.522 | 1.00 | 32.50 |
| 2473 | C | PHE | B | 448 | 15.117 | 88.525 | 28.854 | 1.00 | 33.67 |
| 2474 | O | PHE | B | 448 | 15.650 | 87.488 | 29.228 | 1.00 | 35.26 |
| 2475 | CB | PHE | B | 448 | 15.970 | 89.941 | 27.003 | 1.00 | 35.29 |
| 2476 | CG | PHE | B | 448 | 16.523 | 88.751 | 26.262 | 1.00 | 37.04 |
| 2477 | CD1 | PHE | B | 448 | 15.691 | 87.688 | 25.905 | 1.00 | 36.66 |
| 2478 | CD2 | PHE | B | 448 | 17.886 | 88.666 | 25.973 | 1.00 | 34.95 |
| 2479 | CE1 | PHE | B | 448 | 16.218 | 86.551 | 25.276 | 1.00 | 40.91 |
| 2480 | CE2 | PHE | B | 448 | 18.422 | 87.534 | 25.347 | 1.00 | 35.99 |
| 2481 | CZ | PHE | B | 448 | 17.586 | 86.477 | 24.997 | 1.00 | 35.15 |
| 2482 | N | ALA | B | 449 | 13.804 | 88.648 | 28.704 | 1.00 | 35.81 |
| 2483 | CA | ALA | B | 449 | 12.907 | 87.534 | 28.969 | 1.00 | 37.63 |
| 2484 | C | ALA | B | 449 | 12.347 | 86.983 | 27.662 | 1.00 | 40.96 |
| 2485 | O | ALA | B | 449 | 12.159 | 87.715 | 26.697 | 1.00 | 40.14 |
| 2486 | CB | ALA | B | 449 | 11.769 | 87.977 | 29.870 | 1.00 | 33.60 |
| 2487 | N | THR | B | 450 | 12.091 | 85.682 | 27.652 | 1.00 | 45.40 |
| 2488 | CA | THR | B | 450 | 11.529 | 84.994 | 26.505 | 1.00 | 50.82 |
| 2489 | C | THR | B | 450 | 10.003 | 85.085 | 26.602 | 1.00 | 53.78 |
| 2490 | O | THR | B | 450 | 9.431 | 84.927 | 27.683 | 1.00 | 54.14 |
| 2491 | CB | THR | B | 450 | 11.935 | 83.510 | 26.513 | 1.00 | 51.45 |
| 2492 | OG1 | THR | B | 450 | 13.358 | 83.408 | 26.650 | 1.00 | 54.12 |
| 2493 | CG2 | THR | B | 450 | 11.507 | 82.831 | 25.223 | 1.00 | 55.58 |
| 2494 | N | PRO | B | 451 | 9.325 | 85.340 | 25.474 | 1.00 | 56.74 |
| 2495 | CA | PRO | B | 451 | 7.863 | 85.443 | 25.477 | 1.00 | 59.05 |
| 2496 | C | PRO | B | 451 | 7.209 | 84.173 | 26.020 | 1.00 | 61.00 |
| 2497 | O | PRO | B | 451 | 7.520 | 83.065 | 25.579 | 1.00 | 59.71 |
| 2498 | CB | PRO | B | 451 | 7.527 | 85.667 | 24.002 | 1.00 | 60.52 |
| 2499 | CG | PRO | B | 451 | 8.749 | 86.338 | 23.460 | 1.00 | 61.14 |
| 2500 | CD | PRO | B | 451 | 9.861 | 85.551 | 24.119 | 1.00 | 59.39 |
| 2501 | N | GLU | B | 452 | 6.315 | 84.328 | 26.988 | 1.00 | 64.30 |
| 2502 | CA | GLU | B | 452 | 5.625 | 83.172 | 27.542 | 1.00 | 69.17 |
| 2503 | C | GLU | B | 452 | 4.137 | 83.348 | 27.258 | 1.00 | 72.61 |
| 2504 | O | GLU | B | 452 | 3.394 | 83.933 | 28.043 | 1.00 | 73.59 |
| 2505 | CB | GLU | B | 452 | 5.888 | 83.040 | 29.046 | 1.00 | 65.66 |
| 2506 | CG | GLU | B | 452 | 6.034 | 81.585 | 29.523 | 1.00 | 61.46 |
| 2507 | CD | GLU | B | 452 | 7.283 | 80.883 | 28.967 | 1.00 | 60.52 |
| 2508 | OE1 | GLU | B | 452 | 8.336 | 81.547 | 28.849 | 1.00 | 63.47 |
| 2509 | OE2 | GLU | B | 452 | 7.227 | 79.666 | 28.667 | 1.00 | 53.34 |
| 2510 | N | TRP | B | 453 | 3.731 | 82.837 | 26.103 | 1.00 | 76.75 |
| 2511 | CA | TRP | B | 453 | 2.358 | 82.907 | 25.619 | 1.00 | 81.00 |
| 2512 | C | TRP | B | 453 | 1.573 | 81.714 | 26.187 | 1.00 | 82.30 |
| 2513 | O | TRP | B | 453 | 1.890 | 80.562 | 25.885 | 1.00 | 82.08 |
| 2514 | CB | TRP | B | 453 | 2.409 | 82.863 | 24.090 | 1.00 | 85.65 |
| 2515 | CD | TRP | B | 453 | 1.233 | 83.418 | 23.366 | 1.00 | 89.76 |
| 2516 | CD1 | TRP | B | 453 | 0.679 | 84.661 | 23.512 | 1.00 | 92.34 |
| 2517 | CD2 | TRP | B | 453 | 0.517 | 82.781 | 22.305 | 1.00 | 94.15 |
| 2518 | NE1 | TRP | B | 453 | 0.334 | 84.836 | 22.598 | 1.00 | 94.99 |
| 2519 | CE2 | TRP | B | 453 | 0.455 | 83.696 | 21.845 | 1.00 | 95.56 |
| 2520 | CE3 | TRP | B | 453 | 0.605 | 81.521 | 21.693 | 1.00 | 95.36 |
| 2521 | CZ2 | TRP | B | 453 | 1.335 | 83.392 | 20.800 | 1.00 | 97.14 |
| 2522 | CZ3 | TRP | B | 453 | 0.269 | 81.220 | 20.655 | 1.00 | 95.61 |
| 2523 | CH2 | TRP | B | 453 | 1.226 | 82.153 | 20.220 | 1.00 | 97.12 |
| 2524 | N | PRO | B | 454 | 0.542 | 81.981 | 27.020 | 1.00 | 84.10 |
| 2525 | CA | PRO | B | 454 | 0.331 | 80.994 | 27.678 | 1.00 | 87.24 |
| 2526 | C | PRO | B | 454 | 0.716 | 79.708 | 26.938 | 1.00 | 90.52 |
| 2527 | O | PRO | B | 454 | 0.378 | 79.512 | 25.767 | 1.00 | 89.75 |
| 2528 | CB | PRO | B | 454 | 1.550 | 81.825 | 28.112 | 1.00 | 84.91 |
| 2529 | CG | PRO | B | 454 | 1.417 | 83.125 | 27.360 | 1.00 | 83.62 |
| 2530 | CD | PRO | B | 454 | 0.063 | 83.341 | 27.307 | 1.00 | 84.60 |
| 2531 | N | GLY | B | 455 | 1.455 | 78.849 | 27.636 | 1.00 | 91.72 |
| 2532 | CA | GLY | B | 455 | 1.813 | 77.552 | 27.088 | 1.00 | 94.13 |
| 2533 | C | GLY | B | 455 | 0.532 | 76.870 | 27.499 | 1.00 | 96.28 |
| 2534 | O | CLY | B | 455 | 0.475 | 76.987 | 26.799 | 1.00 | 97.74 |
| 2535 | N | SER | B | 456 | 0.542 | 76.158 | 28.622 | 1.00 | 99.11 |
| 2536 | CA | SER | B | 456 | 0.731 | 75.626 | 29.077 | 1.00 | 96.15 |
| 2537 | C | SER | B | 456 | 1.135 | 74.193 | 29.424 | 1.00 | 91.50 |
| 2538 | O | SER | B | 456 | 0.316 | 73.308 | 29.767 | 1.00 | 88.52 |
| 2539 | CB | SER | B | 456 | 1.222 | 76.508 | 30.221 | 1.00 | 95.05 |
| 2540 | OG | SER | B | 456 | 0.225 | 76.690 | 31.216 | 1.00 | 110.76 |
| 2541 | N | ARG | B | 457 | 2.464 | 74.059 | 29.348 | 1.00 | 87.73 |
| 2542 | CA | ARG | B | 457 | 3.349 | 72.936 | 29.580 | 1.00 | 87.81 |
| 2543 | C | ARG | B | 457 | 4.497 | 73.922 | 29.453 | 1.00 | 86.30 |
| 2544 | O | ARG | B | 457 | 4.602 | 74.574 | 28.403 | 1.00 | 90.57 |
| 2545 | CB | ARG | B | 457 | 3.339 | 71.966 | 28.425 | 1.00 | 94.67 |
| 2546 | N | ASP | B | 458 | 5.341 | 74.088 | 30.473 | 1.00 | 87.32 |
| 2547 | CA | ASP | B | 458 | 6.314 | 75.159 | 30.320 | 1.00 | 80.43 |
| 2548 | C | ASP | B | 458 | 7.637 | 75.178 | 31.020 | 1.00 | 72.83 |
| 2549 | O | ASP | B | 458 | 8.163 | 74.190 | 31.501 | 1.00 | 73.96 |
| 2550 | CB | ASP | B | 458 | 5.615 | 76.450 | 30.715 | 1.00 | 83.19 |
| 2551 | CG | ASP | B | 458 | 5.300 | 76.498 | 32.210 | 1.00 | 85.09 |
| 2552 | OD1 | ASP | B | 458 | 4.694 | 75.535 | 32.722 | 1.00 | 91.25 |
| 2553 | OD2 | ASP | B | 458 | 5.661 | 77.492 | 32.883 | 1.00 | 90.84 |
| 2554 | N | LYS | B | 459 | 8.149 | 76.404 | 31.024 | 1.00 | 66.05 |
| 2555 | CA | LYS | B | 459 | 9.389 | 76.818 | 31.663 | 1.00 | 61.75 |
| 2556 | C | LYS | B | 459 | 9.617 | 78.232 | 31.214 | 1.00 | 56.14 |
| 2557 | O | LYS | B | 459 | 9.952 | 78.456 | 30.054 | 1.00 | 56.54 |
| 2558 | CB | LYS | B | 459 | 10.569 | 75.959 | 31.211 | 1.00 | 68.18 |
| 2559 | CG | LYS | B | 459 | 10.796 | 74.799 | 32.131 | 1.00 | 71.85 |
| 2560 | CD | LYS | B | 459 | 10.737 | 75.194 | 33.601 | 1.00 | 73.79 |
| 2561 | CE | LYS | B | 459 | 10.761 | 73.945 | 34.474 | 1.00 | 76.14 |
| 2562 | NZ | LYS | B | 459 | 10.925 | 74.259 | 35.931 | 1.00 | 79.35 |
| 2563 | N | ARG | B | 460 | 9.406 | 79.194 | 32.099 | 1.00 | 52.32 |
| 2564 | CA | ARG | B | 460 | 9.649 | 80.565 | 31.723 | 1.00 | 47.59 |
| 2565 | C | ARG | B | 460 | 11.164 | 80.740 | 31.810 | 1.00 | 43.34 |
| 2566 | O | ARG | B | 460 | 11.839 | 80.123 | 32.638 | 1.00 | 40.40 |
| 2567 | CB | ARG | B | 460 | 8.874 | 81.496 | 32.644 | 1.00 | 49.27 |
| 2568 | CG | ARG | B | 460 | 7.384 | 81.325 | 32.429 | 1.00 | 56.23 |
| 2569 | CD | ARG | B | 460 | 6.594 | 81.941 | 33.568 | 1.00 | 63.20 |
| 2570 | NE | ARG | B | 460 | 5.174 | 81.603 | 33.504 | 1.00 | 68.02 |
| 2571 | CZ | ARG | B | 460 | 4.645 | 80.455 | 33.920 | 1.00 | 70.46 |
| 2572 | NH1 | ARG | B | 460 | 5.415 | 79.506 | 34.444 | 1.00 | 72.23 |
| 2573 | NH2 | ARG | B | 460 | 3.336 | 80.264 | 33.815 | 1.00 | 73.53 |
| 2574 | N | THR | B | 461 | 11.690 | 81.538 | 30.902 | 1.00 | 39.19 |
| 2575 | CA | THR | B | 461 | 13.126 | 81.721 | 30.822 | 1.00 | 36.81 |
| 2576 | C | THR | B | 461 | 13.596 | 83.153 | 30.699 | 1.00 | 34.03 |
| 2577 | O | THR | B | 461 | 12.993 | 83.969 | 29.990 | 1.00 | 33.96 |
| 2578 | CB | THR | B | 461 | 13.710 | 80.997 | 29.588 | 1.00 | 39.13 |
| 2579 | OG1 | THR | B | 461 | 13.243 | 79.643 | 29.543 | 1.00 | 46.04 |
| 2580 | CG2 | THR | B | 461 | 15.220 | 80.970 | 29.679 | 1.00 | 45.48 |
| 2581 | N | LEU | B | 462 | 14.696 | 83.443 | 31.386 | 1.00 | 29.84 |
| 2582 | CA | LEU | B | 462 | 15.333 | 84.747 | 31.312 | 1.00 | 28.03 |
| 2583 | C | LEU | B | 462 | 16.674 | 84.395 | 30.690 | 1.00 | 27.33 |
| 2584 | O | LEU | B | 462 | 17.186 | 83.293 | 30.893 | 1.00 | 24.47 |
| 2585 | CB | LEU | B | 462 | 15.526 | 85.377 | 32.697 | 1.00 | 25.97 |
| 2586 | CG | LEU | B | 462 | 14.238 | 85.712 | 33.465 | 1.00 | 33.39 |
| 2587 | CD1 | LEU | B | 462 | 14.593 | 86.428 | 34.781 | 1.00 | 32.36 |
| 2588 | CD2 | LEU | B | 462 | 13.337 | 86.586 | 32.617 | 1.00 | 30.72 |
| 2589 | N | ALA | B | 463 | 17.241 | 85.314 | 29.922 | 1.00 | 25.83 |
| 2590 | CA | ALA | B | 463 | 18.489 | 85.023 | 29.264 | 1.00 | 25.65 |
| 2591 | C | ALA | B | 463 | 19.321 | 86.270 | 29.143 | 1.00 | 26.32 |
| 2592 | O | ALA | B | 463 | 18.810 | 87.386 | 29.254 | 1.00 | 27.53 |
| 2593 | CB | ALA | B | 463 | 18.219 | 84.421 | 27.888 | 1.00 | 28.15 |
| 2594 | N | CYS | B | 464 | 20.608 | 86.082 | 28.905 | 1.00 | 21.23 |
| 2595 | CA | CYS | B | 464 | 21.507 | 87.212 | 28.824 | 1.00 | 24.77 |
| 2596 | C | CYS | B | 464 | 22.584 | 87.001 | 27.786 | 1.00 | 22.14 |
| 2597 | O | CYS | B | 464 | 23.169 | 85.917 | 27.698 | 1.00 | 24.66 |
| 2598 | CB | CYS | B | 464 | 22.171 | 87.407 | 30.186 | 1.00 | 22.04 |
| 2599 | SG | CYS | B | 464 | 23.302 | 88.810 | 30.380 | 1.00 | 34.06 |
| 2600 | N | LEU | B | 465 | 22.859 | 88.052 | 27.025 | 1.00 | 22.44 |
| 2601 | CA | LEU | B | 465 | 23.903 | 88.002 | 26.011 | 1.00 | 23.11 |
| 2602 | C | LEU | B | 465 | 24.985 | 88.956 | 26.459 | 1.00 | 22.43 |
| 2603 | O | LEU | B | 465 | 24.702 | 90.117 | 26.748 | 1.00 | 23.73 |
| 2604 | CB | LEU | B | 465 | 23.370 | 88.445 | 24.639 | 1.00 | 22.99 |
| 2605 | CG | LEU | B | 465 | 24.423 | 88.790 | 23.562 | 1.00 | 19.04 |
| 2606 | CD1 | LEU | B | 465 | 25.269 | 87.565 | 23.234 | 1.00 | 21.72 |
| 2607 | CD2 | LEU | B | 465 | 23.716 | 89.253 | 22.282 | 1.00 | 28.23 |
| 2608 | N | ILE | B | 466 | 26.220 | 88.475 | 26.519 | 1.00 | 22.56 |
| 2609 | CA | ILE | B | 466 | 27.332 | 89.323 | 26.931 | 1.00 | 22.11 |
| 2610 | C | ILE | B | 466 | 28.344 | 89.298 | 25.803 | 1.00 | 24.25 |
| 2611 | O | ILE | B | 466 | 28.785 | 88.225 | 25.394 | 1.00 | 24.93 |
| 2612 | CB | ILE | B | 466 | 27.946 | 88.819 | 28.239 | 1.00 | 22.15 |
| 2613 | CG1 | ILE | B | 466 | 26.850 | 88.751 | 29.314 | 1.00 | 23.07 |
| 2614 | CG2 | ILE | B | 466 | 29.075 | 89.778 | 28.684 | 1.00 | 25.24 |
| 2615 | CD1 | ILE | B | 466 | 27.282 | 88.091 | 30.604 | 1.00 | 33.12 |
| 2616 | N | GLN | B | 467 | 28.737 | 90.468 | 25.307 | 1.00 | 24.63 |
| 2617 | CA | GLN | B | 467 | 29.630 | 90.480 | 24.149 | 1.00 | 28.64 |
| 2618 | C | GLN | B | 467 | 30.637 | 91.618 | 24.028 | 1.00 | 31.08 |
| 2619 | O | GLN | B | 467 | 30.685 | 92.529 | 24.865 | 1.00 | 31.00 |
| 2620 | CB | GLN | B | 467 | 28.770 | 90.428 | 22.872 | 1.00 | 25.58 |
| 2621 | CG | GLN | B | 467 | 27.873 | 91.657 | 22.696 | 1.00 | 25.53 |
| 2622 | CD | GLN | B | 467 | 26.907 | 91.556 | 21.501 | 1.00 | 30.72 |
| 2623 | OE1 | GLN | B | 467 | 27.144 | 90.810 | 20.551 | 1.00 | 29.76 |
| 2624 | NE2 | GLN | B | 467 | 25.821 | 92.327 | 21.550 | 1.00 | 32.18 |
| 2625 | N | ASN | B | 468 | 31.453 | 91.517 | 22.977 | 1.00 | 33.71 |
| 2626 | CA | ASN | B | 468 | 32.494 | 92.486 | 22.641 | 1.00 | 36.96 |
| 2627 | C | ASN | B | 468 | 33.601 | 92.607 | 23.677 | 1.00 | 38.20 |
| 2628 | O | ASN | B | 468 | 34.208 | 93.669 | 23.812 | 1.00 | 38.39 |
| 2629 | CB | ASN | B | 468 | 31.881 | 93.869 | 22.424 | 1.00 | 41.06 |
| 2630 | CG | ASN | B | 468 | 30.763 | 93.852 | 21.414 | 1.00 | 46.41 |
| 2631 | OD1 | ASN | B | 468 | 30.879 | 93.228 | 20.359 | 1.00 | 50.82 |
| 2632 | ND2 | ASN | B | 468 | 29.672 | 94.548 | 21.724 | 1.00 | 53.31 |
| 2633 | N | PHE | B | 469 | 33.868 | 91.535 | 24.415 | 1.00 | 35.74 |
| 2634 | CA | PHE | B | 469 | 34.910 | 91.597 | 25.423 | 1.00 | 33.87 |
| 2635 | C | PHE | B | 469 | 36.147 | 90.796 | 25.044 | 1.00 | 33.63 |
| 2636 | O | PHE | B | 469 | 36.087 | 89.873 | 24.240 | 1.00 | 34.78 |
| 2637 | CB | PHE | B | 469 | 34.372 | 91.105 | 26.778 | 1.00 | 29.12 |
| 2638 | CG | PHE | B | 469 | 33.909 | 89.674 | 26.766 | 1.00 | 26.27 |
| 2639 | CD1 | PHE | B | 469 | 34.808 | 88.641 | 26.967 | 1.00 | 22.45 |
| 2640 | CD2 | PHE | B | 469 | 32.566 | 89.362 | 26.561 | 1.00 | 22.06 |
| 2641 | CE1 | PHE | B | 469 | 34.386 | 87.315 | 26.974 | 1.00 | 23.84 |
| 2642 | CE2 | PHE | B | 469 | 32.135 | 88.036 | 26.564 | 1.00 | 28.02 |
| 2643 | CZ | PHE | B | 469 | 33.051 | 87.007 | 26.773 | 1.00 | 23.33 |
| 2644 | N | MET | B | 470 | 37.273 | 91.169 | 25.634 | 1.00 | 35.64 |
| 2645 | CA | MET | B | 470 | 38.535 | 90.480 | 25.413 | 1.00 | 37.62 |
| 2646 | C | MET | B | 470 | 39.496 | 90.916 | 26.504 | 1.00 | 36.36 |
| 2647 | O | MET | B | 470 | 39.478 | 92.071 | 26.923 | 1.00 | 36.33 |
| 2648 | CB | MET | B | 470 | 39.105 | 90.796 | 24.024 | 1.00 | 42.61 |
| 2649 | CG | MET | B | 470 | 39.421 | 92.246 | 23.750 | 1.00 | 50.60 |
| 2650 | SD | MET | B | 470 | 39.856 | 92.459 | 21.987 | 1.00 | 66.08 |
| 2651 | CE | MET | B | 470 | 41.544 | 91.820 | 21.966 | 1.00 | 66.35 |
| 2652 | N | PRO | B | 471 | 40.317 | 89.987 | 27.021 | 1.00 | 34.63 |
| 2653 | CA | PRO | B | 471 | 40.431 | 88.561 | 26.682 | 1.00 | 32.61 |
| 2654 | C | PRO | B | 471 | 39.147 | 87.782 | 26.957 | 1.00 | 31.92 |
| 2655 | O | PRO | B | 471 | 38.164 | 88.356 | 27.415 | 1.00 | 29.59 |
| 2656 | CB | PRO | B | 471 | 41.585 | 88.080 | 27.562 | 1.00 | 33.95 |
| 2657 | CG | PRO | B | 471 | 42.373 | 89.323 | 27.805 | 1.00 | 37.90 |
| 2658 | CD | PRO | B | 471 | 41.309 | 90.354 | 28.042 | 1.00 | 34.78 |
| 2659 | N | GLU | B | 472 | 39.187 | 86.472 | 26.714 | 1.00 | 32.28 |
| 2660 | CA | GLU | B | 472 | 38.020 | 85.599 | 26.866 | 1.00 | 36.49 |
| 2661 | C | GLU | B | 472 | 37.557 | 85.220 | 28.270 | 1.00 | 35.52 |
| 2662 | O | GLU | B | 472 | 36.438 | 84.730 | 28.417 | 1.00 | 34.87 |
| 2663 | CB | GLU | B | 472 | 38.223 | 84.298 | 26.085 | 1.00 | 41.97 |
| 2664 | CG | GLU | B | 472 | 39.294 | 83.387 | 26.677 | 1.00 | 54.78 |
| 2665 | CD | GLU | B | 472 | 39.456 | 82.073 | 25.916 | 1.00 | 62.41 |
| 2666 | OE1 | GLU | B | 472 | 39.021 | 81.997 | 24.744 | 1.00 | 67.26 |
| 2667 | OE2 | GLU | B | 472 | 40.034 | 81.119 | 26.487 | 1.00 | 64.96 |
| 2668 | N | ASP | B | 473 | 38.379 | 85.439 | 29.295 | 1.00 | 34.38 |
| 2669 | CA | ASP | B | 473 | 37.971 | 85.057 | 30.648 | 1.00 | 31.83 |
| 2670 | C | ASP | B | 473 | 36.914 | 85.998 | 31.214 | 1.00 | 31.24 |
| 2671 | O | ASP | B | 473 | 37.072 | 87.220 | 31.199 | 1.00 | 31.80 |
| 2672 | CB | ASP | B | 473 | 39.180 | 84.979 | 31.584 | 1.00 | 34.73 |
| 2673 | CG | ASP | B | 473 | 40.147 | 83.854 | 31.196 | 1.00 | 40.83 |
| 2674 | OD1 | ASP | B | 473 | 39.676 | 82.759 | 30.804 | 1.00 | 44.18 |
| 2675 | OD2 | ASP | B | 473 | 41.378 | 84.061 | 31.290 | 1.00 | 40.17 |
| 2676 | N | ILE | B | 474 | 35.826 | 85.416 | 31.702 | 1.00 | 26.96 |
| 2677 | CA | ILE | B | 474 | 34.744 | 86.207 | 32.246 | 1.00 | 25.39 |
| 2678 | C | ILE | B | 474 | 33.885 | 85.375 | 33.208 | 1.00 | 24.74 |
| 2679 | O | ILE | B | 474 | 33.776 | 84.164 | 33.069 | 1.00 | 25.20 |
| 2680 | CB | ILE | B | 474 | 33.862 | 86.758 | 31.092 | 1.00 | 23.81 |
| 2681 | CG1 | ILE | B | 474 | 32.925 | 87.852 | 31.598 | 1.00 | 22.23 |
| 2682 | CG2 | ILE | B | 474 | 33.062 | 85.626 | 30.453 | 1.00 | 27.10 |
| 2683 | CD1 | ILE | B | 474 | 32.144 | 88.536 | 30.450 | 1.00 | 25.30 |
| 2684 | N | SER | B | 475 | 33.312 | 86.030 | 34.209 | 1.00 | 23.27 |
| 2685 | CA | SER | B | 475 | 32.430 | 85.348 | 35.148 | 1.00 | 22.17 |
| 2686 | C | SER | B | 475 | 31.066 | 85.991 | 34.952 | 1.00 | 21.71 |
| 2687 | O | SER | B | 475 | 30.951 | 87.207 | 34.876 | 1.00 | 20.84 |
| 2688 | CB | SER | B | 475 | 32.879 | 85.551 | 36.593 | 1.00 | 20.43 |
| 2689 | 00 | SER | B | 475 | 34.164 | 85.011 | 36.806 | 1.00 | 30.00 |
| 2690 | N | VAL | B | 476 | 30.042 | 85.158 | 34.887 | 1.00 | 21.76 |
| 2691 | CA | VAL | B | 476 | 28.694 | 85.620 | 34.675 | 1.00 | 21.72 |
| 2692 | C | VAL | B | 476 | 27.875 | 85.165 | 35.851 | 1.00 | 23.56 |
| 2693 | O | VAL | B | 476 | 28.036 | 84.045 | 36.320 | 1.00 | 25.95 |
| 2694 | CB | VAL | B | 476 | 28.087 | 84.994 | 33.385 | 1.00 | 21.56 |
| 2695 | CG1 | VAL | B | 476 | 26.625 | 85.443 | 33.222 | 1.00 | 18.70 |
| 2696 | CG2 | VAL | B | 476 | 28.915 | 85.403 | 32.166 | 1.00 | 20.99 |
| 2697 | N | GLN | B | 477 | 26.994 | 86.026 | 36.336 | 1.00 | 25.16 |
| 2698 | CA | GLN | B | 477 | 26.172 | 85.635 | 37.460 | 1.00 | 26.65 |
| 2699 | C | GLN | B | 477 | 24.833 | 86.337 | 37.438 | 1.00 | 26.62 |
| 2700 | O | GLN | B | 477 | 24.709 | 87.440 | 36.925 | 1.00 | 26.40 |
| 2701 | CB | GLN | B | 477 | 26.917 | 85.899 | 38.775 | 1.00 | 31.51 |
| 2702 | CG | GLN | B | 477 | 27.348 | 87.311 | 39.011 | 1.00 | 44.27 |
| 2703 | CD | GLN | B | 477 | 28.505 | 87.412 | 40.016 | 1.00 | 47.76 |
| 2704 | OE1 | GLN | B | 477 | 28.814 | 88.496 | 40.502 | 1.00 | 50.57 |
| 2705 | NE2 | GLN | B | 477 | 29.151 | 86.283 | 40.313 | 1.00 | 47.32 |
| 2706 | N | TRP | B | 478 | 23.820 | 85.659 | 37.954 | 1.00 | 27.41 |
| 2707 | CA | TRP | B | 478 | 22.488 | 86.225 | 38.026 | 1.00 | 28.36 |
| 2708 | C | TRP | B | 478 | 22.192 | 86.656 | 39.465 | 1.00 | 30.10 |
| 2709 | O | TRP | B | 478 | 22.660 | 86.033 | 40.428 | 1.00 | 25.31 |
| 2710 | CB | TRP | B | 478 | 21.461 | 85.196 | 37.567 | 1.00 | 28.76 |
| 2711 | CG | TRP | B | 478 | 21.502 | 84.951 | 36.087 | 1.00 | 34.11 |
| 2712 | CD1 | TRP | B | 478 | 22.313 | 84.078 | 35.415 | 1.00 | 31.25 |
| 2713 | CD2 | TRP | B | 478 | 20.722 | 85.620 | 35.094 | 1.00 | 31.01 |
| 2714 | NE1 | TRP | B | 478 | 22.085 | 84.164 | 34.069 | 1.00 | 27.71 |
| 2715 | CE2 | TRP | B | 478 | 21.113 | 85.104 | 33.841 | 1.00 | 31.95 |
| 2716 | CE3 | TRP | B | 478 | 19.729 | 86.607 | 35.140 | 1.00 | 34.16 |
| 2717 | CZ2 | TRP | B | 478 | 20.542 | 85.545 | 32.637 | 1.00 | 25.26 |
| 2718 | CZ3 | TRP | B | 478 | 19.161 | 87.044 | 33.947 | 1.00 | 31.64 |
| 2719 | CH2 | TRP | B | 478 | 19.571 | 86.511 | 32.713 | 1.00 | 28.89 |
| 2720 | N | LEU | B | 479 | 21.418 | 87.723 | 39.602 | 1.00 | 32.48 |
| 2721 | CA | LEU | B | 479 | 21.049 | 88.245 | 40.913 | 1.00 | 37.36 |
| 2722 | C | LEU | B | 479 | 19.584 | 88.657 | 40.960 | 1.00 | 40.42 |
| 2723 | O | LEU | B | 479 | 19.040 | 89.152 | 39.977 | 1.00 | 40.14 |
| 2724 | CB | LEU | B | 479 | 21.878 | 89.478 | 41.252 | 1.00 | 39.83 |
| 2725 | CG | LEU | B | 479 | 23.346 | 89.556 | 40.842 | 1.00 | 44.57 |
| 2726 | CD1 | LEU | B | 479 | 23.890 | 90.887 | 41.290 | 1.00 | 52.98 |
| 2727 | CD2 | LEU | B | 479 | 24.130 | 88.439 | 41.466 | 1.00 | 50.71 |
| 2728 | N | HIS | B | 480 | 18.947 | 88.448 | 42.102 | 1.00 | 43.05 |
| 2729 | CA | HIS | B | 480 | 17.572 | 88.877 | 42.276 | 1.00 | 49.20 |
| 2730 | C | HIS | B | 480 | 17.746 | 90.136 | 43.116 | 1.00 | 52.73 |
| 2731 | O | HIS | B | 480 | 17.705 | 90.091 | 44.347 | 1.00 | 52.90 |
| 2732 | CB | HIS | B | 480 | 16.754 | 87.844 | 43.043 | 1.00 | 52.25 |
| 2733 | CG | HIS | B | 480 | 15.341 | 88.270 | 43.286 | 1.00 | 57.22 |
| 2734 | ND1 | HIS | B | 480 | 14.337 | 88.075 | 42.364 | 1.00 | 61.37 |
| 2735 | CD2 | HIS | B | 480 | 14.778 | 88.942 | 44.319 | 1.00 | 59.16 |
| 2736 | CE1 | HIS | B | 480 | 13.216 | 88.607 | 42.815 | 1.00 | 59.88 |
| 2737 | NE2 | HIS | B | 480 | 13.456 | 89.140 | 44.000 | 1.00 | 66.11 |
| 2738 | N | ASN | B | 481 | 17.971 | 91.253 | 42.430 | 1.00 | 56.65 |
| 2739 | CA | ASN | B | 481 | 18.206 | 92.543 | 43.070 | 1.00 | 59.93 |
| 2740 | C | ASN | B | 481 | 19.660 | 92.591 | 43.519 | 1.00 | 59.65 |
| 2741 | O | ASN | B | 481 | 20.571 | 92.569 | 42.695 | 1.00 | 61.04 |
| 2742 | CB | ASN | B | 481 | 17.288 | 92.751 | 44.282 | 1.00 | 64.65 |
| 2743 | CG | ASN | B | 481 | 15.852 | 93.022 | 43.890 | 1.00 | 72.38 |
| 2744 | OG1 | ASN | B | 481 | 15.563 | 93.964 | 43.147 | 1.00 | 74.96 |
| 2745 | ND2 | ASN | B | 481 | 14.938 | 92.199 | 44.393 | 1.00 | 75.60 |
| 2746 | N | GLU | B | 482 | 19.872 | 92.625 | 44.827 | 1.00 | 58.97 |
| 2747 | CA | GLU | B | 482 | 21.213 | 92.697 | 45.396 | 1.00 | 58.55 |
| 2748 | C | GLU | B | 482 | 21.815 | 91.346 | 45.782 | 1.00 | 55.30 |
| 2749 | O | GLU | B | 482 | 23.013 | 91.253 | 46.044 | 1.00 | 57.27 |
| 2750 | CB | GLU | B | 482 | 21.176 | 93.607 | 46.627 | 1.00 | 65.42 |
| 2751 | CG | GLU | B | 482 | 19.760 | 93.813 | 47.172 | 1.00 | 73.94 |
| 2752 | CD | GLU | B | 482 | 19.735 | 94.473 | 48.532 | 1.00 | 80.29 |
| 2753 | OE1 | GLU | B | 482 | 20.168 | 93.830 | 49.513 | 1.00 | 86.31 |
| 2754 | OE2 | GLU | B | 482 | 19.284 | 95.633 | 48.620 | 1.00 | 82.73 |
| 2755 | N | VAL | B | 483 | 20.997 | 90.301 | 45.814 | 1.00 | 50.25 |
| 2756 | CA | VAL | B | 483 | 21.487 | 88.988 | 46.211 | 1.00 | 45.78 |
| 2757 | C | VAL | B | 483 | 21.733 | 88.025 | 45.052 | 1.00 | 43.04 |
| 2758 | O | VAL | B | 483 | 20.880 | 87.834 | 44.198 | 1.00 | 41.34 |
| 2759 | CB | VAL | B | 483 | 20.527 | 88.339 | 47.233 | 1.00 | 43.76 |
| 2760 | CG1 | VAL | B | 483 | 19.129 | 88.283 | 46.668 | 1.00 | 46.25 |
| 2761 | CG2 | VAL | B | 483 | 21.011 | 86.946 | 47.594 | 1.00 | 41.37 |
| 2762 | N | GLN | B | 484 | 22.912 | 87.412 | 45.050 | 1.00 | 42.30 |
| 2763 | CA | GLN | B | 484 | 23.300 | 86.474 | 44.005 | 1.00 | 43.21 |
| 2764 | C | GLN | B | 484 | 22.658 | 85.091 | 44.141 | 1.00 | 42.48 |
| 2765 | O | GLN | B | 484 | 22.538 | 84.545 | 45.244 | 1.00 | 41.64 |
| 2766 | CB | GLN | B | 484 | 24.826 | 86.331 | 43.971 | 1.00 | 45.96 |
| 2767 | CG | GLN | B | 484 | 25.324 | 85.269 | 42.999 | 1.00 | 54.43 |
| 2768 | CD | GLN | B | 484 | 26.840 | 85.173 | 42.941 | 1.00 | 59.02 |
| 2769 | OE1 | GLN | B | 484 | 27.391 | 84.200 | 42.423 | 1.00 | 59.71 |
| 2770 | NE2 | GLN | B | 484 | 27.522 | 86.190 | 43.464 | 1.00 | 61.46 |
| 2771 | N | LEU | B | 485 | 22.239 | 84.539 | 43.006 | 1.00 | 38.99 |
| 2772 | CA | LEU | B | 485 | 21.618 | 83.218 | 42.953 | 1.00 | 39.17 |
| 2773 | C | LEU | B | 485 | 22.700 | 82.142 | 42.853 | 1.00 | 40.35 |
| 2774 | O | LEU | B | 485 | 23.822 | 82.417 | 42.430 | 1.00 | 37.39 |
| 2775 | CB | LEU | B | 485 | 20.705 | 83.107 | 41.725 | 1.00 | 37.16 |
| 2776 | CG | LEU | B | 485 | 19.579 | 84.140 | 41.602 | 1.00 | 39.66 |
| 2777 | CD1 | LEU | B | 485 | 18.836 | 83.930 | 40.293 | 1.00 | 39.30 |
| 2778 | CD2 | LEU | B | 485 | 18.644 | 84.020 | 42.795 | 1.00 | 40.56 |
| 2779 | N | PRO | B | 486 | 22.373 | 80.900 | 43.242 | 1.00 | 42.55 |
| 2780 | CA | PRO | B | 486 | 23.366 | 79.828 | 43.158 | 1.00 | 46.30 |
| 2781 | C | PRO | B | 486 | 23.750 | 79.660 | 41.688 | 1.00 | 49.32 |
| 2782 | O | PRO | B | 486 | 22.916 | 79.852 | 40.806 | 1.00 | 48.81 |
| 2783 | CB | PRO | B | 486 | 22.606 | 78.609 | 43.675 | 1.00 | 45.64 |
| 2784 | CG | PRO | B | 486 | 21.532 | 79.187 | 44.528 | 1.00 | 45.29 |
| 2785 | CD | PRO | B | 486 | 21.095 | 80.394 | 43.769 | 1.00 | 40.65 |
| 2786 | N | ASP | B | 487 | 24.997 | 79.290 | 41.429 | 1.00 | 53.39 |
| 2787 | CA | ASP | B | 487 | 25.465 | 79.093 | 40.062 | 1.00 | 57.75 |
| 2788 | C | ASP | B | 487 | 24.660 | 77.997 | 39.350 | 1.00 | 57.25 |
| 2789 | O | ASP | B | 487 | 24.205 | 78.176 | 38.213 | 1.00 | 57.75 |
| 2790 | CB | ASP | B | 487 | 26.944 | 78.708 | 40.077 | 1.00 | 67.82 |
| 2791 | CG | ASP | B | 487 | 27.577 | 78.779 | 38.703 | 1.00 | 76.17 |
| 2792 | OD1 | ASP | B | 487 | 26.945 | 78.309 | 37.734 | 1.00 | 84.77 |
| 2793 | OD2 | ASP | B | 487 | 28.711 | 79.297 | 38.592 | 1.00 | 86.94 |
| 2794 | N | ALA | B | 488 | 24.478 | 76.873 | 40.035 | 1.00 | 54.64 |
| 2795 | CA | ALA | B | 488 | 23.752 | 75.729 | 39.493 | 1.00 | 53.45 |
| 2796 | C | ALA | B | 488 | 22.421 | 76.069 | 38.837 | 1.00 | 52.74 |
| 2797 | O | ALA | B | 488 | 21.843 | 75.241 | 38.134 | 1.00 | 53.06 |
| 2798 | CB | ALA | B | 488 | 23.530 | 74.708 | 40.585 | 1.00 | 53.75 |
| 2799 | N | ARG | B | 489 | 21.938 | 77.287 | 39.056 | 1.00 | 50.96 |
| 2800 | CA | ARG | B | 489 | 20.660 | 77.704 | 38.491 | 1.00 | 47.84 |
| 2801 | C | ARG | B | 489 | 20.705 | 78.164 | 37.036 | 1.00 | 45.10 |
| 2802 | O | ARG | B | 489 | 19.681 | 78.158 | 36.363 | 1.00 | 43.37 |
| 2803 | CB | ARG | B | 489 | 20.046 | 78.808 | 39.354 | 1.00 | 48.99 |
| 2804 | CG | ARG | B | 489 | 19.232 | 78.294 | 40.524 | 1.00 | 50.01 |
| 2805 | CD | ARG | B | 489 | 17.761 | 78.427 | 40.227 | 1.00 | 50.36 |
| 2806 | NE | ARG | B | 489 | 17.187 | 79.581 | 40.905 | 1.00 | 56.65 |
| 2807 | CZ | ARG | B | 489 | 16.048 | 80.166 | 40.557 | 1.00 | 58.23 |
| 2808 | NH1 | ARG | B | 489 | 15.352 | 79.714 | 39.522 | 1.00 | 61.07 |
| 2809 | NH2 | ARO | B | 489 | 15.594 | 81.193 | 41.260 | 1.00 | 59.63 |
| 2810 | N | HIS | B | 490 | 21.872 | 78.573 | 36.551 | 1.00 | 42.41 |
| 2811 | CA | HIS | B | 490 | 21.965 | 79.024 | 35.166 | 1.00 | 41.63 |
| 2812 | C | HIS | B | 490 | 22.952 | 78.210 | 34.334 | 1.00 | 39.71 |
| 2813 | O | HIS | B | 490 | 23.768 | 77.471 | 34.865 | 1.00 | 41.30 |
| 2814 | CB | HIS | B | 490 | 22.348 | 80.508 | 35.100 | 1.00 | 42.40 |
| 2815 | CD | HIS | B | 490 | 23.720 | 80.807 | 35.614 | 1.00 | 43.27 |
| 2816 | ND1 | HIS | B | 490 | 23.966 | 81.177 | 36.919 | 1.00 | 42.27 |
| 2817 | CD2 | HIS | B | 490 | 24.926 | 80.779 | 34.999 | 1.00 | 44.41 |
| 2818 | CE1 | HIS | B | 490 | 25.262 | 81.364 | 37.086 | 1.00 | 39.88 |
| 2819 | NE2 | HIS | B | 490 | 25.868 | 81.130 | 35.934 | 1.00 | 45.37 |
| 2820 | N | SER | B | 491 | 22.858 | 78.355 | 33.020 | 1.00 | 37.20 |
| 2821 | CA | SER | B | 491 | 23.734 | 77.654 | 32.095 | 1.00 | 36.36 |
| 2822 | C | SER | B | 491 | 24.401 | 78.685 | 31.173 | 1.00 | 33.06 |
| 2823 | O | SER | B | 491 | 23.721 | 79.428 | 30.461 | 1.00 | 33.77 |
| 2824 | CB | SER | B | 491 | 22.910 | 76.651 | 31.288 | 1.00 | 35.42 |
| 2825 | OG | SER | B | 491 | 23.694 | 76.053 | 30.282 | 1.00 | 43.65 |
| 2826 | N | THR | B | 492 | 25.728 | 78.722 | 31.188 | 1.00 | 29.90 |
| 2827 | CA | THR | B | 492 | 26.495 | 79.673 | 30.383 | 1.00 | 29.91 |
| 2828 | C | THR | B | 492 | 27.322 | 78.977 | 29.287 | 1.00 | 29.20 |
| 2829 | O | THR | B | 492 | 27.996 | 77.991 | 29.552 | 1.00 | 30.85 |
| 2830 | CB | THR | B | 492 | 27.447 | 80.506 | 31.298 | 1.00 | 29.85 |
| 2831 | OG1 | THR | B | 492 | 26.670 | 81.197 | 32.281 | 1.00 | 35.86 |
| 2832 | CG2 | THR | B | 492 | 28.234 | 81.541 | 30.498 | 1.00 | 28.05 |
| 2833 | N | THR | B | 493 | 27.281 | 79.505 | 28.065 | 1.00 | 29.49 |
| 2834 | CA | THR | B | 493 | 28.032 | 78.913 | 26.957 | 1.00 | 30.19 |
| 2835 | C | THR | B | 493 | 29.501 | 79.295 | 27.056 | 1.00 | 32.39 |
| 2836 | O | THR | B | 493 | 29.863 | 80.210 | 27.786 | 1.00 | 30.70 |
| 2837 | CB | THR | B | 493 | 27.502 | 79.378 | 25.568 | 1.00 | 27.82 |
| 2838 | OG1 | THR | B | 493 | 27.592 | 80.806 | 25.460 | 1.00 | 30.48 |
| 2839 | CG2 | THR | B | 493 | 26.067 | 78.953 | 25.372 | 1.00 | 22.03 |
| 2840 | N | GLN | B | 494 | 30.347 | 78.586 | 26.322 | 1.00 | 35.09 |
| 2841 | CA | GLN | B | 494 | 31.779 | 78.879 | 26.330 | 1.00 | 38.19 |
| 2842 | C | GLN | B | 494 | 32.032 | 80.091 | 25.449 | 1.00 | 36.48 |
| 2843 | O | GLN | B | 494 | 31.360 | 80.270 | 24.432 | 1.00 | 35.75 |
| 2844 | CB | GLN | B | 494 | 32.581 | 77.696 | 25.775 | 1.00 | 43.09 |
| 2845 | CD | GLN | B | 494 | 32.502 | 76.433 | 26.610 | 1.00 | 58.35 |
| 2846 | CD | GLN | B | 494 | 32.929 | 76.666 | 28.042 | 1.00 | 65.11 |
| 2847 | OE1 | GLN | B | 494 | 34.003 | 77.209 | 28.303 | 1.00 | 71.67 |
| 2848 | NE2 | GLN | B | 494 | 32.088 | 76.255 | 28.982 | 1.00 | 71.87 |
| 2849 | N | PRO | B | 495 | 33.002 | 80.939 | 25.828 | 1.00 | 36.89 |
| 2850 | CA | PRO | B | 495 | 33.322 | 82.130 | 25.037 | 1.00 | 38.37 |
| 2851 | C | PRO | B | 495 | 33.609 | 81.737 | 23.587 | 1.00 | 40.80 |
| 2852 | O | PRO | B | 495 | 34.314 | 80.763 | 23.327 | 1.00 | 40.76 |
| 2853 | CB | PRO | B | 495 | 34.557 | 82.680 | 25.729 | 1.00 | 37.83 |
| 2854 | CG | PRO | B | 495 | 34.315 | 82.300 | 27.174 | 1.00 | 40.41 |
| 2855 | CD | PRO | B | 495 | 33.809 | 80.884 | 27.061 | 1.00 | 35.81 |
| 2856 | N | ARG | B | 496 | 33.040 | 82.480 | 22.650 | 1.00 | 42.05 |
| 2857 | CA | ARG | B | 496 | 33.255 | 82.208 | 21.240 | 1.00 | 47.69 |
| 2858 | C | ARG | B | 496 | 33.772 | 83.454 | 20.542 | 1.00 | 51.66 |
| 2859 | O | ARG | B | 496 | 33.283 | 84.558 | 20.784 | 1.00 | 51.95 |
| 2860 | CB | ARO | B | 496 | 31.954 | 81.751 | 20.584 | 1.00 | 45.81 |
| 2861 | CG | ARG | B | 496 | 31.599 | 80.317 | 20.892 | 1.00 | 51.53 |
| 2862 | CD | ARG | B | 496 | 30.217 | 79.947 | 20.391 | 1.00 | 53.72 |
| 2863 | NE | ARO | B | 496 | 30.175 | 78.554 | 19.963 | 1.00 | 57.50 |
| 2864 | CZ | ARG | B | 496 | 30.608 | 78.122 | 18.783 | 1.00 | 58.59 |
| 2865 | NH1 | ARG | B | 496 | 31.111 | 78.974 | 17.901 | 1.00 | 57.22 |
| 2866 | NH2 | ARG | B | 496 | 30.542 | 76.832 | 18.488 | 1.00 | 65.31 |
| 2867 | N | LYS | B | 497 | 34.773 | 83.281 | 19.687 | 1.00 | 56.98 |
| 2868 | CA | LYS | B | 497 | 35.335 | 84.408 | 18.953 | 1.00 | 61.91 |
| 2869 | C | LYS | B | 497 | 34.301 | 84.867 | 17.955 | 1.00 | 63.97 |
| 2870 | O | LYS | B | 497 | 33.300 | 84.195 | 17.729 | 1.00 | 64.61 |
| 2871 | CB | LYS | B | 497 | 36.587 | 84.003 | 18.172 | 1.00 | 66.35 |
| 2872 | CG | LYS | B | 497 | 37.848 | 83.781 | 18.987 | 1.00 | 74.14 |
| 2873 | CD | LYS | B | 497 | 38.993 | 83.359 | 18.066 | 1.00 | 80.69 |
| 2874 | CE | LYS | B | 497 | 40.304 | 83.192 | 18.814 | 1.00 | 84.48 |
| 2875 | NZ | LYS | B | 497 | 41.385 | 82.739 | 17.889 | 1.00 | 88.26 |
| 2876 | N | THR | B | 498 | 34.555 | 86.017 | 17.352 | 1.00 | 67.67 |
| 2877 | CA | THR | B | 498 | 33.669 | 86.572 | 16.342 | 1.00 | 71.26 |
| 2878 | C | THR | B | 498 | 34.550 | 87.284 | 15.340 | 1.00 | 73.04 |
| 2879 | O | THR | B | 498 | 35.743 | 86.990 | 15.236 | 1.00 | 72.83 |
| 2880 | CB | THR | B | 498 | 32.679 | 87.585 | 16.945 | 1.00 | 71.80 |
| 2881 | OG1 | THR | B | 498 | 33.392 | 88.555 | 17.721 | 1.00 | 71.58 |
| 2882 | CG | THR | B | 498 | 31.670 | 86.878 | 17.819 | 1.00 | 73.61 |
| 2883 | N | LYS | B | 499 | 33.962 | 88.220 | 14.605 | 1.00 | 75.57 |
| 2884 | CA | LYS | B | 499 | 34.705 | 88.993 | 13.620 | 1.00 | 77.62 |
| 2885 | C | LYS | B | 499 | 35.427 | 90.142 | 14.321 | 1.00 | 77.92 |
| 2886 | O | LYS | B | 499 | 34.791 | 91.096 | 14.768 | 1.00 | 79.19 |
| 2887 | CB | LYS | B | 499 | 33.752 | 89.558 | 12.567 | 1.00 | 80.22 |
| 2888 | CG | LYS | B | 499 | 33.172 | 88.531 | 11.604 | 1.00 | 81.04 |
| 2889 | CD | LYS | B | 499 | 34.203 | 88.093 | 10.571 | 1.00 | 83.26 |
| 2890 | CE | LYS | B | 499 | 33.569 | 87.232 | 9.487 | 1.00 | 84.81 |
| 2891 | NZ | LYS | B | 499 | 34.545 | 86.864 | 8.425 | 1.00 | 86.04 |
| 2892 | N | GLY | B | 500 | 36.748 | 90.034 | 14.433 | 1.00 | 76.91 |
| 2893 | CA | GLY | B | 500 | 37.544 | 91.077 | 15.061 | 1.00 | 75.83 |
| 2894 | C | GLY | B | 500 | 37.141 | 91.560 | 16.445 | 1.00 | 75.14 |
| 2895 | O | GLY | B | 500 | 37.971 | 91.576 | 17.355 | 1.00 | 75.52 |
| 2896 | N | SER | B | 501 | 35.882 | 91.958 | 16.605 | 1.00 | 73.08 |
| 2897 | CA | SER | B | 501 | 35.370 | 92.468 | 17.875 | 1.00 | 71.65 |
| 2898 | C | BER | B | 501 | 36.008 | 91.836 | 19.106 | 1.00 | 68.64 |
| 2899 | O | SER | B | 501 | 36.690 | 92.515 | 19.877 | 1.00 | 71.13 |
| 2900 | CB | SER | B | 501 | 33.854 | 92.279 | 17.953 | 1.00 | 74.43 |
| 2901 | OG | SER | B | 501 | 33.524 | 90.933 | 18.241 | 1.00 | 80.92 |
| 2902 | N | GLY | B | 502 | 35.786 | 90.540 | 19.290 | 1.00 | 63.14 |
| 2903 | CA | GLY | B | 502 | 36.344 | 89.859 | 20.443 | 1.00 | 54.62 |
| 2904 | C | GLY | B | 502 | 35.552 | 88.618 | 20.803 | 1.00 | 48.04 |
| 2905 | O | GLY | B | 502 | 35.469 | 87.683 | 20.006 | 1.00 | 48.08 |
| 2906 | N | PHE | B | 503 | 34.958 | 88.599 | 21.994 | 1.00 | 40.50 |
| 2907 | CA | PHE | B | 503 | 34.189 | 87.427 | 22.408 | 1.00 | 33.81 |
| 2908 | C | PHE | B | 503 | 32.773 | 87.692 | 22.888 | 1.00 | 28.39 |
| 2909 | O | PHE | B | 503 | 32.408 | 88.811 | 23.244 | 1.00 | 26.35 |
| 2910 | CB | PHE | B | 503 | 34.919 | 86.662 | 23.517 | 1.00 | 35.09 |
| 2911 | CG | PHE | B | 503 | 36.263 | 86.148 | 23.119 | 1.00 | 34.16 |
| 2912 | CD1 | PHE | B | 503 | 37.381 | 86.970 | 23.174 | 1.00 | 35.05 |
| 2913 | CD2 | PHE | B | 503 | 36.411 | 84.845 | 22.670 | 1.00 | 35.90 |
| 2914 | CE1 | PHE | B | 503 | 38.633 | 86.501 | 22.783 | 1.00 | 36.00 |
| 2915 | CE2 | PHE | B | 503 | 37.656 | 84.362 | 22.274 | 1.00 | 40.04 |
| 2916 | CZ | PHE | B | 503 | 38.771 | 85.192 | 22.329 | 1.00 | 38.82 |
| 2917 | N | PHE | B | 504 | 31.975 | 86.633 | 22.884 | 1.00 | 26.16 |
| 2918 | CA | PHE | B | 504 | 30.612 | 86.709 | 23.369 | 1.00 | 25.00 |
| 2919 | C | PHE | B | 504 | 30.286 | 85.418 | 24.096 | 1.00 | 26.57 |
| 2920 | O | PHE | B | 504 | 30.892 | 84.379 | 23.833 | 1.00 | 26.73 |
| 2921 | CB | PHE | B | 504 | 29.616 | 86.960 | 22.242 | 1.00 | 22.46 |
| 2922 | CG | PHE | B | 504 | 29.330 | 85.766 | 21.382 | 1.00 | 27.70 |
| 2923 | CD1 | PHE | B | 504 | 28.260 | 84.926 | 21.667 | 1.00 | 28.94 |
| 2924 | CD2 | PHE | B | 504 | 30.086 | 85.517 | 20.243 | 1.00 | 31.34 |
| 2925 | CE1 | PHE | B | 504 | 27.938 | 83.857 | 20.827 | 1.00 | 30.08 |
| 2926 | CE2 | PHE | B | 504 | 29.772 | 84.449 | 19.396 | 1.00 | 32.65 |
| 2927 | CZ | PHE | B | 504 | 28.691 | 83.621 | 19.697 | 1.00 | 32.80 |
| 2928 | N | VAL | B | 505 | 29.330 | 85.509 | 25.015 | 1.00 | 23.68 |
| 2929 | CA | VAL | B | 505 | 28.895 | 84.389 | 25.814 | 1.00 | 24.67 |
| 2930 | C | VAL | B | 505 | 27.385 | 84.559 | 26.028 | 1.00 | 23.86 |
| 2931 | O | VAL | B | 505 | 26.875 | 85.672 | 26.051 | 1.00 | 21.24 |
| 2932 | CB | VAL | B | 505 | 29.674 | 84.392 | 27.161 | 1.00 | 27.95 |
| 2933 | CG1 | VAL | B | 505 | 29.136 | 85.466 | 28.080 | 1.00 | 25.61 |
| 2934 | CG2 | VAL | B | 505 | 29.643 | 83.042 | 27.786 | 1.00 | 33.51 |
| 2935 | N | PHE | B | 506 | 26.664 | 83.454 | 26.141 | 1.00 | 26.20 |
| 2936 | CA | PHE | B | 506 | 25.218 | 83.514 | 26.346 | 1.00 | 26.10 |
| 2937 | C | PHE | B | 506 | 24.855 | 82.754 | 27.621 | 1.00 | 24.83 |
| 2938 | O | PHE | B | 506 | 25.369 | 81.672 | 27.859 | 1.00 | 25.52 |
| 2939 | CB | PHE | B | 506 | 24.496 | 82.875 | 25.161 | 1.00 | 28.18 |
| 2940 | CG | PHE | B | 506 | 23.007 | 82.842 | 25.303 | 1.00 | 35.33 |
| 2941 | CD1 | PHE | B | 506 | 22.245 | 83.980 | 25.080 | 1.00 | 42.56 |
| 2942 | CD2 | PHE | B | 506 | 22.359 | 81.663 | 25.657 | 1.00 | 40.04 |
| 2943 | CE1 | PHE | B | 506 | 20.854 | 83.940 | 25.208 | 1.00 | 44.70 |
| 2944 | CE2 | PHE | B | 506 | 20.977 | 81.613 | 25.789 | 1.00 | 40.54 |
| 2945 | CZ | PHE | B | 506 | 20.223 | 82.749 | 25.564 | 1.00 | 42.12 |
| 2946 | N | SER | B | 507 | 23.961 | 83.310 | 28.427 | 1.00 | 24.69 |
| 2947 | CA | SER | B | 507 | 23.550 | 82.659 | 29.674 | 1.00 | 24.73 |
| 2948 | C | SER | B | 507 | 22.022 | 82.535 | 29.745 | 1.00 | 26.40 |
| 2949 | O | SER | B | 507 | 21.308 | 83.487 | 29.491 | 1.00 | 25.99 |
| 2950 | CB | SER | B | 507 | 24.079 | 83.449 | 30.879 | 1.00 | 22.19 |
| 2951 | OC | SER | B | 507 | 23.653 | 82.860 | 32.091 | 1.00 | 30.30 |
| 2952 | N | ARG | B | 508 | 21.538 | 81.345 | 30.091 | 1.00 | 26.98 |
| 2953 | CA | ARG | B | 508 | 20.104 | 81.055 | 30.187 | 1.00 | 25.95 |
| 2954 | C | ARG | B | 508 | 19.689 | 80.749 | 31.637 | 1.00 | 24.97 |
| 2955 | O | ARG | B | 508 | 20.350 | 79.979 | 32.315 | 1.00 | 24.35 |
| 2956 | CB | ARG | B | 508 | 19.799 | 79.845 | 29.310 | 1.00 | 21.97 |
| 2957 | CG | ARG | B | 508 | 18.365 | 79.351 | 29.344 | 1.00 | 25.32 |
| 2958 | CD | ARG | B | 508 | 18.228 | 78.126 | 28.444 | 1.00 | 25.67 |
| 2959 | NE | ARG | B | 508 | 16.842 | 77.702 | 28.312 | 1.00 | 36.00 |
| 2960 | CZ | ARG | B | 508 | 16.243 | 76.843 | 29.128 | 1.00 | 36.06 |
| 2961 | NH1 | ARG | B | 508 | 16.918 | 76.307 | 30.137 | 1.00 | 34.19 |
| 2962 | NH2 | ARG | B | 508 | 14.967 | 76.538 | 28.943 | 1.00 | 34.70 |
| 2963 | N | LEU | B | 509 | 18.583 | 81.331 | 32.099 | 1.00 | 26.44 |
| 2964 | CA | LEU | B | 509 | 18.121 | 81.108 | 33.479 | 1.00 | 28.45 |
| 2965 | C | LEU | B | 509 | 16.627 | 80.806 | 33.560 | 1.00 | 28.87 |
| 2966 | O | LEU | B | 509 | 15.803 | 81.707 | 33.401 | 1.00 | 30.99 |
| 2967 | CB | LEU | B | 509 | 18.423 | 82.341 | 34.342 | 1.00 | 27.28 |
| 2968 | CG | LEU | B | 509 | 17.867 | 82.330 | 35.779 | 1.00 | 31.90 |
| 2969 | CD1 | LEU | B | 509 | 18.615 | 81.316 | 36.647 | 1.00 | 29.48 |
| 2970 | CD2 | LEU | B | 509 | 18.001 | 83.714 | 36.368 | 1.00 | 28.89 |
| 2971 | N | GLU | B | 510 | 16.277 | 79.546 | 33.812 | 1.00 | 31.92 |
| 2972 | CA | GLU | B | 510 | 14.872 | 79.152 | 33.911 | 1.00 | 34.67 |
| 2973 | C | GLU | B | 510 | 14.310 | 79.595 | 35.255 | 1.00 | 36.35 |
| 2974 | O | GLU | B | 510 | 14.948 | 79.401 | 36.288 | 1.00 | 34.66 |
| 2975 | CB | GLU | B | 510 | 14.725 | 77.630 | 33.757 | 1.00 | 41.07 |
| 2976 | CG | GLU | B | 510 | 15.217 | 77.090 | 32.404 | 1.00 | 54.35 |
| 2977 | CD | GLU | B | 510 | 14.925 | 75.603 | 32.189 | 1.00 | 58.86 |
| 2978 | OE1 | GLU | B | 510 | 15.236 | 74.789 | 33.087 | 1.00 | 61.22 |
| 2979 | OE2 | GLU | B | 510 | 14.393 | 75.251 | 31.111 | 1.00 | 61.34 |
| 2980 | N | VAL | B | 511 | 13.121 | 80.194 | 35.248 | 1.00 | 36.50 |
| 2981 | CA | VAL | B | 511 | 12.531 | 80.665 | 36.494 | 1.00 | 38.05 |
| 2982 | C | VAL | B | 511 | 11.084 | 80.230 | 36.692 | 1.00 | 42.41 |
| 2983 | O | VAL | B | 511 | 10.395 | 79.856 | 35.732 | 1.00 | 41.92 |
| 2984 | CB | VAL | B | 511 | 12.588 | 82.197 | 36.592 | 1.00 | 31.84 |
| 2985 | CG1 | VAL | B | 511 | 14.015 | 82.673 | 36.431 | 1.00 | 32.42 |
| 2986 | CG2 | VAL | B | 511 | 11.687 | 82.813 | 35.547 | 1.00 | 26.87 |
| 2987 | N | THR | B | 512 | 10.633 | 80.290 | 37.946 | 1.00 | 43.58 |
| 2988 | CA | THR | B | 512 | 9.269 | 79.903 | 38.292 | 1.00 | 48.37 |
| 2989 | C | THR | B | 512 | 8.294 | 80.998 | 37.899 | 1.00 | 50.53 |
| 2990 | O | THR | B | 512 | 8.706 | 82.113 | 37.584 | 1.00 | 51.77 |
| 2991 | CB | THR | B | 512 | 9.125 | 79.644 | 39.797 | 1.00 | 48.57 |
| 2992 | OC1 | THR | B | 512 | 9.460 | 80.834 | 40.519 | 1.00 | 46.96 |
| 2993 | CG2 | THR | B | 512 | 10.046 | 78.515 | 40.234 | 1.00 | 45.82 |
| 2994 | N | ARG | B | 513 | 7.002 | 80.678 | 37.912 | 1.00 | 53.29 |
| 2995 | CA | ARG | B | 513 | 5.971 | 81.648 | 37.556 | 1.00 | 55.54 |
| 2996 | C | ARG | B | 513 | 6.043 | 82.819 | 38.523 | 1.00 | 55.38 |
| 2997 | O | ARG | B | 513 | 5.859 | 83.977 | 38.138 | 1.00 | 55.22 |
| 2998 | CB | ARG | B | 513 | 4.582 | 81.006 | 37.636 | 1.00 | 59.23 |
| 2999 | CG | ARG | B | 513 | 3.563 | 81.601 | 36.672 | 1.00 | 65.98 |
| 3000 | CD | ARG | B | 513 | 3.326 | 83.081 | 36.916 | 1.00 | 74.12 |
| 3001 | NE | ARG | B | 513 | 2.773 | 83.742 | 35.735 | 1.00 | 80.97 |
| 3002 | CZ | ARG | B | 513 | 2.403 | 85.019 | 35.687 | 1.00 | 83.64 |
| 3003 | NH1 | ARG | B | 513 | 2.516 | 85.791 | 36.760 | 1.00 | 86.02 |
| 3004 | NH2 | ARG | B | 513 | 1.927 | 85.528 | 34.557 | 1.00 | 85.44 |
| 3005 | N | ALA | B | 514 | 6.315 | 82.501 | 39.783 | 1.00 | 55.03 |
| 3006 | CA | ALA | B | 514 | 6.414 | 83.504 | 40.832 | 1.00 | 55.46 |
| 3007 | C | ALA | B | 514 | 7.496 | 84.539 | 40.529 | 1.00 | 56.10 |
| 3008 | O | ALA | B | 514 | 7.210 | 85.734 | 40.426 | 1.00 | 56.61 |
| 3009 | CB | ALA | B | 514 | 6.692 | 82.825 | 42.173 | 1.00 | 54.05 |
| 3010 | N | GLU | B | 515 | 8.741 | 84.091 | 40.388 | 1.00 | 55.46 |
| 3011 | CA | GLU | B | 515 | 9.819 | 85.025 | 40.105 | 1.00 | 55.34 |
| 3012 | C | GLU | B | 515 | 9.649 | 85.651 | 38.729 | 1.00 | 54.83 |
| 3013 | O | GLU | B | 515 | 10.190 | 86.723 | 38.456 | 1.00 | 52.33 |
| 3014 | CB | GLU | B | 515 | 11.187 | 84.335 | 40.232 | 1.00 | 57.76 |
| 3015 | CG | GLU | B | 515 | 11.221 | 82.884 | 39.788 | 1.00 | 62.58 |
| 3016 | CD | GLU | B | 515 | 12.518 | 82.184 | 40.163 | 1.00 | 63.27 |
| 3017 | OE1 | GLU | B | 515 | 13.035 | 82.432 | 41.271 | 1.00 | 64.89 |
| 3018 | OE2 | GLU | B | 515 | 13.014 | 81.372 | 39.359 | 1.00 | 66.59 |
| 3019 | N | TRP | B | 516 | 8.884 | 84.992 | 37.866 | 1.00 | 55.79 |
| 3020 | CA | TRP | B | 516 | 8.644 | 85.528 | 36.534 | 1.00 | 57.81 |
| 3021 | C | TRP | B | 516 | 7.778 | 86.782 | 36.636 | 1.00 | 58.51 |
| 3022 | O | TRP | B | 516 | 7.778 | 87.612 | 35.726 | 1.00 | 57.71 |
| 3023 | CB | TRP | B | 516 | 7.951 | 84.499 | 35.634 | 1.00 | 61.48 |
| 3024 | CG | TRP | B | 516 | 8.056 | 84.862 | 34.187 | 1.00 | 66.54 |
| 3025 | CD1 | TRP | B | 516 | 9.172 | 84.777 | 33.403 | 1.00 | 67.26 |
| 3026 | CD2 | TRP | B | 516 | 7.039 | 85.460 | 33.375 | 1.00 | 69.56 |
| 3027 | NE1 | TRP | B | 516 | 8.915 | 85.291 | 32.154 | 1.00 | 71.76 |
| 3028 | CE2 | TRP | B | 516 | 7.613 | 85.718 | 32.110 | 1.00 | 70.88 |
| 3029 | CE3 | TRP | B | 516 | 5.700 | 85.807 | 33.593 | 1.00 | 70.94 |
| 3030 | CZ2 | TRP | B | 516 | 6.894 | 86.308 | 31.066 | 1.00 | 73.50 |
| 3031 | CZ3 | TRP | B | 516 | 4.982 | 86.396 | 32.552 | 1.00 | 74.37 |
| 3032 | CH2 | TRP | B | 516 | 5.584 | 86.639 | 31.304 | 1.00 | 74.71 |
| 3033 | N | GLU | B | 517 | 7.047 | 86.918 | 37.744 | 1.00 | 59.66 |
| 3034 | CA | GLU | B | 517 | 6.191 | 88.085 | 37.972 | 1.00 | 61.72 |
| 3035 | C | GLU | B | 517 | 7.035 | 89.299 | 38.354 | 1.00 | 59.87 |
| 3036 | O | GLU | B | 517 | 6.606 | 90.441 | 38.196 | 1.00 | 58.60 |
| 3037 | CB | GLU | B | 517 | 5.182 | 87.818 | 39.097 | 1.00 | 68.82 |
| 3038 | CG | GLU | B | 517 | 4.284 | 86.612 | 38.879 | 1.00 | 80.60 |
| 3039 | CD | GLU | B | 517 | 3.242 | 86.452 | 39.976 | 1.00 | 87.42 |
| 3040 | OE1 | GLU | B | 517 | 3.369 | 87.128 | 41.021 | 1.00 | 94.80 |
| 3041 | OE2 | GLU | B | 517 | 2.301 | 85.645 | 39.800 | 1.00 | 95.35 |
| 3042 | N | GLN | B | 518 | 8.234 | 89.045 | 38.867 | 1.00 | 57.92 |
| 3043 | CA | GLN | B | 518 | 9.132 | 90.120 | 39.264 | 1.00 | 55.74 |
| 3044 | C | GLN | B | 518 | 10.416 | 90.057 | 38.436 | 1.00 | 54.32 |
| 3045 | O | GLN | B | 518 | 11.498 | 90.393 | 38.924 | 1.00 | 52.85 |
| 3046 | CB | GLN | B | 518 | 9.454 | 90.000 | 40.745 | 1.00 | 54.10 |
| 3047 | N | LYS | B | 519 | 10.287 | 89.646 | 37.176 | 1.00 | 52.69 |
| 3048 | CA | LYS | B | 519 | 11.448 | 89.518 | 36.309 | 1.00 | 52.35 |
| 3049 | C | LYS | B | 519 | 12.206 | 90.819 | 36.159 | 1.00 | 51.69 |
| 3050 | O | LYS | B | 519 | 13.397 | 90.822 | 35.854 | 1.00 | 49.61 |
| 3051 | CB | LYS | B | 519 | 11.039 | 88.983 | 34.937 | 1.00 | 54.31 |
| 3052 | CG | LYS | B | 519 | 10.205 | 89.912 | 34.088 | 1.00 | 55.80 |
| 3053 | CD | LYS | B | 519 | 9.723 | 89.162 | 32.859 | 1.00 | 60.32 |
| 3054 | CE | LYS | B | 519 | 8.794 | 90.002 | 32.011 | 1.00 | 64.05 |
| 3055 | NZ | LYS | B | 519 | 8.223 | 89.191 | 30.898 | 1.00 | 70.92 |
| 3056 | N | ASP | B | 520 | 11.511 | 91.924 | 36.388 | 1.00 | 52.66 |
| 3057 | CA | ASP | B | 520 | 12.105 | 93.251 | 36.288 | 1.00 | 54.22 |
| 3058 | C | ASP | B | 520 | 13.209 | 93.396 | 37.336 | 1.00 | 53.07 |
| 3059 | O | ASP | B | 520 | 14.075 | 94.264 | 37.229 | 1.00 | 52.75 |
| 3060 | CB | ASP | B | 520 | 11.032 | 94.311 | 36.537 | 1.00 | 61.14 |
| 3061 | CG | ASP | B | 520 | 9.620 | 93.779 | 36.308 | 1.00 | 71.19 |
| 3062 | OD1 | ASP | B | 520 | 9.300 | 93.407 | 35.155 | 1.00 | 76.53 |
| 3063 | OD2 | ASP | B | 520 | 8.834 | 93.729 | 37.283 | 1.00 | 72.86 |
| 3064 | N | GLU | B | 521 | 13.177 | 92.533 | 38.343 | 1.00 | 51.01 |
| 3065 | CA | GLU | B | 521 | 14.157 | 92.582 | 39.416 | 1.00 | 50.49 |
| 3066 | C | GLU | B | 521 | 15.394 | 91.714 | 39.201 | 1.00 | 47.73 |
| 3067 | O | GLU | B | 521 | 16.372 | 91.838 | 39.935 | 1.00 | 48.58 |
| 3068 | CE | GLU | B | 521 | 13.493 | 92.200 | 40.738 | 1.00 | 53.54 |
| 3069 | CG | GLU | B | 521 | 12.427 | 93.177 | 41.199 | 1.00 | 60.11 |
| 3070 | CD | GLU | B | 521 | 11.705 | 92.699 | 42.442 | 1.00 | 65.41 |
| 3071 | OE1 | GLU | B | 521 | 12.361 | 92.497 | 43.486 | 1.00 | 67.98 |
| 3072 | OE2 | GLU | B | 521 | 10.471 | 92.521 | 42.374 | 1.00 | 73.58 |
| 3073 | N | PHE | B | 522 | 15.359 | 90.828 | 38.213 | 1.00 | 42.66 |
| 3074 | CA | PHE | B | 522 | 16.519 | 89.985 | 37.963 | 1.00 | 38.90 |
| 3075 | C | PHE | B | 522 | 17.583 | 90.753 | 37.208 | 1.00 | 35.93 |
| 3076 | O | PHE | B | 522 | 17.276 | 91.579 | 36.345 | 1.00 | 38.85 |
| 3077 | CB | PHE | B | 522 | 16.122 | 88.716 | 37.214 | 1.00 | 36.56 |
| 3078 | CG | PHE | B | 522 | 15.419 | 87.710 | 38.081 | 1.00 | 39.04 |
| 3079 | CD1 | PHE | B | 522 | 14.106 | 87.927 | 38.495 | 1.00 | 42.28 |
| 3080 | CD2 | PHE | B | 522 | 16.087 | 86.574 | 38.539 | 1.00 | 36.78 |
| 3081 | CE1 | PHE | B | 522 | 13.468 | 87.029 | 39.362 | 1.00 | 44.30 |
| 3082 | CE2 | PHE | B | 522 | 15.461 | 85.673 | 39.405 | 1.00 | 40.92 |
| 3083 | CZ | PHE | B | 522 | 14.146 | 85.901 | 39.817 | 1.00 | 39.51 |
| 3084 | N | ILE | B | 523 | 18.837 | 90.492 | 37.555 | 1.00 | 31.27 |
| 3085 | CA | ILE | B | 523 | 19.959 | 91.173 | 36.944 | 1.00 | 29.65 |
| 3086 | C | ILE | B | 523 | 21.049 | 90.211 | 36.483 | 1.00 | 29.13 |
| 3087 | O | ILE | B | 523 | 21.360 | 89.236 | 37.164 | 1.00 | 27.36 |
| 3088 | CB | ILE | B | 523 | 20.567 | 92.180 | 37.937 | 1.00 | 34.21 |
| 3089 | CD1 | ILE | B | 523 | 19.528 | 93.251 | 38.282 | 1.00 | 37.38 |
| 3090 | CG | ILE | B | 523 | 21.827 | 92.796 | 37.364 | 1.00 | 34.94 |
| 3091 | CD1 | ILE | B | 523 | 19.981 | 94.227 | 39.354 | 1.00 | 38.56 |
| 3092 | N | CYS | B | 524 | 21.610 | 90.498 | 35.311 | 1.00 | 26.34 |
| 3093 | CA | CYS | B | 524 | 22.686 | 89.699 | 34.739 | 1.00 | 27.10 |
| 3094 | C | CYS | B | 524 | 23.946 | 90.500 | 34.962 | 1.00 | 26.67 |
| 3095 | O | CYS | B | 524 | 24.010 | 91.655 | 34.569 | 1.00 | 27.58 |
| 3096 | CB | CYS | B | 524 | 22.473 | 89.497 | 33.226 | 1.00 | 24.27 |
| 3097 | SG | CYS | B | 524 | 23.823 | 88.626 | 32.363 | 1.00 | 37.60 |
| 3098 | N | ARG | B | 525 | 24.951 | 89.886 | 35.572 | 1.00 | 26.37 |
| 3099 | CA | ARG | B | 525 | 26.194 | 90.576 | 35.856 | 1.00 | 26.35 |
| 3100 | C | ARG | B | 525 | 27.419 | 89.887 | 35.304 | 1.00 | 26.72 |
| 3101 | O | ARG | B | 525 | 27.561 | 88.671 | 35.400 | 1.00 | 29.04 |
| 3102 | CB | ARG | B | 525 | 26.376 | 90.748 | 37.361 | 1.00 | 29.81 |
| 3103 | CG | ARG | B | 525 | 27.713 | 91.393 | 37.728 | 1.00 | 35.40 |
| 3104 | CD | ARG | B | 525 | 27.652 | 92.058 | 39.099 | 1.00 | 46.51 |
| 3105 | NE | ARG | B | 525 | 27.796 | 91.113 | 40.193 | 1.00 | 50.77 |
| 3106 | CZ | ARG | B | 525 | 27.461 | 91.379 | 41.451 | 1.00 | 55.09 |
| 3107 | NH1 | ARG | B | 525 | 26.951 | 92.565 | 41.763 | 1.00 | 54.58 |
| 3108 | NH2 | ARG | B | 525 | 27.650 | 90.464 | 42.397 | 1.00 | 56.77 |
| 3109 | N | ALA | B | 526 | 28.311 | 90.680 | 34.731 | 1.00 | 25.38 |
| 3110 | CA | ALA | B | 526 | 29.528 | 90.142 | 34.180 | 1.00 | 25.38 |
| 3111 | C | ALA | B | 526 | 30.721 | 90.728 | 34.925 | 1.00 | 25.99 |
| 3112 | O | ALA | B | 526 | 30.751 | 91.933 | 35.210 | 1.00 | 25.50 |
| 3113 | CB | ALA | B | 526 | 29.629 | 90.484 | 32.696 | 1.00 | 25.98 |
| 3114 | N | VAL | B | 527 | 31.684 | 89.872 | 35.259 | 1.00 | 23.84 |
| 3115 | CA | VAL | B | 527 | 32.895 | 90.333 | 35.902 | 1.00 | 25.15 |
| 3116 | C | VAL | B | 527 | 33.972 | 90.078 | 34.880 | 1.00 | 26.30 |
| 3117 | O | VAL | B | 527 | 34.119 | 88.958 | 34.394 | 1.00 | 26.20 |
| 3118 | CB | VAL | B | 527 | 33.235 | 89.557 | 37.177 | 1.00 | 24.43 |
| 3119 | CG1 | VAL | B | 527 | 34.588 | 89.990 | 37.661 | 1.00 | 26.95 |
| 3120 | CG2 | VAL | B | 527 | 32.191 | 89.819 | 38.251 | 1.00 | 28.13 |
| 3121 | N | HIS | B | 528 | 34.738 | 91.115 | 34.569 | 1.00 | 26.24 |
| 3122 | CA | HIS | B | 528 | 35.772 | 91.018 | 33.564 | 1.00 | 29.14 |
| 3123 | C | HIS | B | 528 | 36.822 | 92.102 | 33.805 | 1.00 | 31.86 |
| 3124 | O | HIS | B | 528 | 36.509 | 93.216 | 34.239 | 1.00 | 31.05 |
| 3125 | CB | HIS | B | 528 | 35.121 | 91.172 | 32.179 | 1.00 | 29.03 |
| 3126 | CG | HIS | B | 528 | 36.077 | 91.082 | 31.035 | 1.00 | 29.34 |
| 3127 | ND1 | HIS | B | 528 | 36.934 | 92.109 | 30.696 | 1.00 | 33.33 |
| 3128 | CD2 | HIS | B | 528 | 36.317 | 90.090 | 30.144 | 1.00 | 28.93 |
| 3129 | CE1 | HIS | B | 528 | 37.655 | 91.755 | 29.651 | 1.00 | 26.07 |
| 3130 | NE2 | HIS | B | 528 | 37.302 | 90.530 | 29.295 | 1.00 | 32.19 |
| 3131 | N | GLU | B | 529 | 38.064 | 91.757 | 33.501 | 1.00 | 34.08 |
| 3132 | CA | GLU | B | 529 | 39.207 | 92.636 | 33.676 | 1.00 | 39.76 |
| 3133 | C | GLU | B | 529 | 39.110 | 94.037 | 33.066 | 1.00 | 41.50 |
| 3134 | O | GLU | B | 529 | 39.410 | 95.020 | 33.740 | 1.00 | 41.48 |
| 3135 | CB | GLU | B | 529 | 40.462 | 91.959 | 33.122 | 1.00 | 43.03 |
| 3136 | CG | GLU | B | 529 | 41.665 | 92.898 | 33.086 | 1.00 | 56.19 |
| 3137 | CD | GLU | B | 529 | 42.827 | 92.361 | 32.274 | 1.00 | 62.66 |
| 3138 | OE1 | GLU | B | 529 | 43.812 | 93.111 | 32.093 | 1.00 | 63.52 |
| 3139 | OE2 | GLU | B | 529 | 42.758 | 91.198 | 31.819 | 1.00 | 65.43 |
| 3140 | N | ALA | B | 530 | 38.703 | 94.121 | 31.800 | 1.00 | 42.51 |
| 3141 | CA | ALA | B | 530 | 38.622 | 95.399 | 31.094 | 1.00 | 46.03 |
| 3142 | C | ALA | B | 530 | 37.495 | 96.331 | 31.504 | 1.00 | 49.29 |
| 3143 | O | ALA | B | 530 | 37.447 | 97.476 | 31.056 | 1.00 | 50.87 |
| 3144 | CB | ALA | B | 530 | 38.557 | 95.157 | 29.587 | 1.00 | 39.71 |
| 3145 | N | ALA | B | 531 | 36.595 | 95.849 | 32.352 | 1.00 | 52.89 |
| 3146 | CA | ALA | B | 531 | 35.467 | 96.651 | 32.798 | 1.00 | 56.14 |
| 3147 | C | ALA | B | 531 | 35.801 | 97.570 | 33.976 | 1.00 | 59.66 |
| 3148 | O | ALA | B | 531 | 35.357 | 97.342 | 35.096 | 1.00 | 61.00 |
| 3149 | CB | ALA | B | 531 | 34.305 | 95.736 | 33.162 | 1.00 | 52.83 |
| 3150 | N | SER | B | 532 | 36.588 | 98.608 | 33.730 | 1.00 | 63.07 |
| 3151 | CA | SER | B | 532 | 36.927 | 99.544 | 34.795 | 1.00 | 66.03 |
| 3152 | C | SER | B | 532 | 35.822 | 100.594 | 34.850 | 1.00 | 66.08 |
| 3153 | O | SER | B | 532 | 35.317 | 101.031 | 33.814 | 1.00 | 65.35 |
| 3154 | CB | SER | B | 532 | 38.270 | 100.216 | 34.507 | 1.00 | 68.98 |
| 3155 | OG | SER | B | 532 | 38.184 | 101.074 | 33.384 | 1.00 | 74.64 |
| 3156 | N | PRO | B | 533 | 35.427 | 101.019 | 36.057 | 1.00 | 66.51 |
| 3157 | CA | PRO | B | 533 | 35.887 | 100.669 | 37.404 | 1.00 | 65.99 |
| 3158 | C | PRO | B | 533 | 35.374 | 99.349 | 37.983 | 1.00 | 63.48 |
| 3159 | O | PRO | B | 533 | 34.327 | 98.840 | 37.582 | 1.00 | 64.33 |
| 3160 | CB | PRO | B | 533 | 35.397 | 101.843 | 38.227 | 1.00 | 69.06 |
| 3161 | CG | PRO | B | 533 | 34.044 | 102.061 | 37.629 | 1.00 | 71.08 |
| 3162 | CD | PRO | B | 533 | 34.358 | 102.030 | 36.137 | 1.00 | 70.23 |
| 3163 | N | SER | B | 534 | 36.134 | 98.817 | 38.935 | 1.00 | 59.21 |
| 3164 | CA | SER | B | 534 | 35.796 | 97.591 | 39.648 | 1.00 | 55.01 |
| 3165 | C | SER | B | 534 | 35.452 | 96.326 | 38.852 | 1.00 | 50.27 |
| 3166 | O | SER | B | 534 | 34.725 | 95.470 | 39.347 | 1.00 | 48.58 |
| 3167 | CB | SER | B | 534 | 34.666 | 97.894 | 40.625 | 1.00 | 58.20 |
| 3168 | OG | SER | B | 534 | 33.587 | 98.526 | 39.962 | 1.00 | 63.36 |
| 3169 | N | GLN | B | 535 | 35.984 | 96.213 | 37.639 | 1.00 | 44.89 |
| 3170 | CA | GLN | B | 535 | 35.771 | 95.057 | 36.770 | 1.00 | 42.88 |
| 3171 | C | GLN | B | 535 | 34.395 | 94.406 | 36.823 | 1.00 | 41.55 |
| 3172 | O | GLN | B | 535 | 34.279 | 93.181 | 36.806 | 1.00 | 40.80 |
| 3173 | CB | GLN | B | 535 | 36.831 | 93.967 | 37.020 | 1.00 | 37.08 |
| 3174 | CG | GLN | B | 535 | 37.869 | 94.298 | 38.044 | 1.00 | 47.40 |
| 3175 | CD | GLN | B | 535 | 38.684 | 95.499 | 37.669 | 1.00 | 44.36 |
| 3176 | OE1 | GLN | B | 535 | 38.862 | 96.407 | 38.475 | 1.00 | 47.59 |
| 3177 | NE2 | GLN | B | 535 | 39.185 | 95.521 | 36.436 | 1.00 | 54.70 |
| 3178 | N | THR | B | 536 | 33.348 | 95.213 | 36.874 | 1.00 | 39.97 |
| 3179 | CA | THR | B | 536 | 32.012 | 94.650 | 36.893 | 1.00 | 40.62 |
| 3180 | C | THR | B | 536 | 31.025 | 95.487 | 36.077 | 1.00 | 37.57 |
| 3181 | O | THR | B | 536 | 31.042 | 96.707 | 36.125 | 1.00 | 40.85 |
| 3182 | CB | THR | B | 536 | 31.508 | 94.463 | 38.353 | 1.00 | 42.38 |
| 3183 | OG1 | THR | B | 536 | 30.112 | 94.142 | 38.337 | 1.00 | 44.59 |
| 3184 | CG2 | THR | B | 536 | 31.730 | 95.719 | 39.168 | 1.00 | 49.65 |
| 3185 | N | VAL | B | 537 | 30.190 | 94.812 | 35.299 | 1.00 | 35.29 |
| 3186 | CA | VAL | B | 537 | 29.182 | 95.453 | 34.468 | 1.00 | 31.25 |
| 3187 | C | VAL | B | 537 | 27.922 | 94.604 | 34.563 | 1.00 | 29.01 |
| 3188 | O | VAL | B | 537 | 27.985 | 93.382 | 34.401 | 1.00 | 30.11 |
| 3189 | CB | VAL | B | 537 | 29.604 | 95.494 | 32.983 | 1.00 | 34.80 |
| 3190 | CG1 | VAL | B | 537 | 28.516 | 96.148 | 32.161 | 1.00 | 35.52 |
| 3191 | CG2 | VAL | B | 537 | 30.888 | 96.259 | 32.822 | 1.00 | 37.19 |
| 3192 | N | GLN | B | 538 | 26.780 | 95.235 | 34.808 | 1.00 | 26.73 |
| 3193 | CA | GLN | B | 538 | 25.533 | 94.493 | 34.933 | 1.00 | 26.74 |
| 3194 | C | GLN | B | 538 | 24.329 | 95.224 | 34.368 | 1.00 | 27.53 |
| 3195 | O | GLN | B | 538 | 24.351 | 96.431 | 34.198 | 1.00 | 29.89 |
| 3196 | CB | GLN | B | 538 | 25.265 | 94.176 | 36.400 | 1.00 | 28.48 |
| 3197 | CG | GLN | B | 538 | 24.976 | 95.392 | 37.266 | 1.00 | 25.88 |
| 3198 | CD | GLN | B | 538 | 24.747 | 95.005 | 38.722 | 1.00 | 28.51 |
| 3199 | OE1 | GLN | B | 538 | 25.498 | 94.210 | 39.290 | 1.00 | 31.66 |
| 3200 | NE2 | GLN | B | 538 | 23.715 | 95.565 | 39.328 | 1.00 | 27.39 |
| 3201 | N | ARG | B | 539 | 23.266 | 94.487 | 34.087 | 1.00 | 28.05 |
| 3202 | CA | ARG | B | 539 | 22.070 | 95.107 | 33.565 | 1.00 | 30.72 |
| 3203 | C | ARG | B | 539 | 20.858 | 94.312 | 33.986 | 1.00 | 31.33 |
| 3204 | O | ARG | B | 539 | 20.897 | 93.083 | 34.023 | 1.00 | 32.42 |
| 3205 | CB | ARG | B | 539 | 22.137 | 95.180 | 32.039 | 1.00 | 36.30 |
| 3206 | CG | ARG | B | 539 | 21.124 | 96.112 | 31.414 | 1.00 | 45.60 |
| 3207 | CD | ARG | B | 539 | 21.771 | 96.859 | 30.270 | 1.00 | 57.97 |
| 3208 | NE | ARG | B | 539 | 23.009 | 97.482 | 30.729 | 1.00 | 69.46 |
| 3209 | CZ | ARG | B | 539 | 23.855 | 98.147 | 29.950 | 1.00 | 78.18 |
| 3210 | NH1 | ARG | B | 539 | 23.604 | 98.284 | 28.653 | 1.00 | 83.01 |
| 3211 | NH2 | ARG | B | 539 | 24.959 | 98.673 | 30.470 | 1.00 | 81.89 |
| 3212 | N | ALA | B | 540 | 19.782 | 95.022 | 34.301 | 1.00 | 31.92 |
| 3213 | CA | ALA | B | 540 | 18.546 | 94.393 | 34.717 | 1.00 | 34.50 |
| 3214 | C | ALA | B | 540 | 17.826 | 93.909 | 33.466 | 1.00 | 36.68 |
| 3215 | O | ALA | B | 540 | 18.068 | 94.410 | 32.361 | 1.00 | 34.62 |
| 3216 | CB | ALA | B | 540 | 17.693 | 95.391 | 35.479 | 1.00 | 33.91 |
| 3217 | N | VAL | B | 541 | 16.974 | 92.906 | 33.640 | 1.00 | 39.73 |
| 3218 | CA | VAL | B | 541 | 16.214 | 92.346 | 32.528 | 1.00 | 44.47 |
| 3219 | C | VAL | B | 541 | 15.351 | 93.436 | 31.882 | 1.00 | 46.91 |
| 3220 | O | VAL | B | 541 | 14.476 | 94.005 | 32.527 | 1.00 | 44.53 |
| 3221 | CB | VAL | B | 541 | 15.305 | 91.185 | 33.010 | 1.00 | 42.49 |
| 3222 | CG1 | VAL | B | 541 | 14.242 | 90.885 | 31.970 | 1.00 | 47.96 |
| 3223 | CG2 | VAL | B | 541 | 16.140 | 89.945 | 33.269 | 1.00 | 42.43 |
| 3224 | N | SER | B | 542 | 15.603 | 93.731 | 30.611 | 1.00 | 52.25 |
| 3225 | CA | SER | B | 542 | 14.829 | 94.760 | 29.926 | 1.00 | 60.05 |
| 3226 | C | SER | B | 542 | 13.426 | 94.226 | 29.679 | 1.00 | 64.25 |
| 3227 | O | SER | B | 542 | 13.247 | 93.050 | 29.357 | 1.00 | 65.22 |
| 3228 | CB | SER | B | 542 | 15.490 | 95.157 | 28.600 | 1.00 | 60.43 |
| 3229 | OG | SER | B | 542 | 15.524 | 94.070 | 27.696 | 1.00 | 65.30 |
| 3230 | N | VAL | B | 543 | 12.432 | 95.090 | 29.844 | 1.00 | 68.19 |
| 3231 | CA | VAL | B | 543 | 11.043 | 94.694 | 29.656 | 1.00 | 72.45 |
| 3232 | C | VAL | B | 543 | 10.277 | 95.772 | 28.898 | 1.00 | 74.32 |
| 3233 | O | VAL | B | 543 | 9.621 | 96.630 | 29.497 | 1.00 | 75.99 |
| 3234 | CB | VAL | B | 543 | 10.353 | 94.452 | 31.016 | 1.00 | 74.41 |
| 3235 | CG1 | VAL | B | 543 | 8.895 | 94.070 | 30.801 | 1.00 | 76.37 |
| 3236 | CG2 | VAL | B | 543 | 11.086 | 93.367 | 31.782 | 1.00 | 75.78 |

Yet another embodiment of the present invention is a 3-D model of a Fc-Cε3/Cε4 region that substantially represents the atomic coordinates specified (i.e., listed) in Table 3.

TABLE 3
Atomic coordinates of 1FP5_dimer.pdb with water

| ATOM | ATOM | | | | | | | | |
|---|---|---|---|---|---|---|---|---|---|
| # | TYPE | RES | CHN | # | X | Y | Z | OCC | B |
| 1 | N | VAL | A | 336 | 46.157 | 62.618 | 17.991 | 1.00 | 58.93 |
| 2 | CA | VAL | A | 336 | 45.400 | 61.812 | 16.993 | 1.00 | 60.44 |
| 3 | C | VAL | A | 336 | 44.013 | 61.427 | 17.501 | 1.00 | 60.07 |
| 4 | O | VAL | A | 336 | 43.847 | 60.389 | 18.142 | 1.00 | 61.48 |
| 5 | CB | VAL | A | 336 | 46.155 | 60.521 | 16.647 | 1.00 | 60.81 |
| 6 | CG1 | VAL | A | 336 | 45.464 | 59.806 | 15.500 | 1.00 | 61.71 |
| 7 | CG2 | VAL | A | 336 | 47.590 | 60.845 | 16.302 | 1.00 | 64.73 |
| 8 | N | SER | A | 337 | 43.017 | 62.257 | 17.209 | 1.00 | 57.95 |
| 9 | CA | SER | A | 337 | 41.655 | 61.983 | 17.648 | 1.00 | 56.60 |
| 10 | C | SER | A | 337 | 40.683 | 61.842 | 16.476 | 1.00 | 55.17 |
| 11 | O | SER | A | 337 | 40.981 | 62.262 | 15.352 | 1.00 | 54.55 |
| 12 | CB | SER | A | 337 | 41.185 | 63.078 | 18.603 | 1.00 | 57.92 |
| 13 | OG | SER | A | 337 | 41.489 | 64.356 | 18.087 | 1.00 | 64.70 |
| 14 | N | ALA | A | 338 | 39.527 | 61.238 | 16.743 | 1.00 | 52.40 |
| 15 | CA | ALA | A | 338 | 38.522 | 61.010 | 15.711 | 1.00 | 50.69 |
| 16 | C | ALA | A | 338 | 37.109 | 61.300 | 16.192 | 1.00 | 50.25 |
| 17 | O | ALA | A | 338 | 36.772 | 61.062 | 17.354 | 1.00 | 51.17 |
| 18 | CB | ALA | A | 338 | 38.611 | 59.573 | 15.211 | 1.00 | 50.21 |
| 19 | N | TYR | A | 339 | 36.281 | 61.808 | 15.284 | 1.00 | 48.22 |
| 20 | CA | TYR | A | 339 | 34.899 | 62.139 | 15.605 | 1.00 | 46.82 |
| 21 | C | TYR | A | 339 | 33.990 | 61.766 | 14.431 | 1.00 | 44.51 |
| 22 | O | TYR | A | 339 | 34.372 | 61.889 | 13.268 | 1.00 | 42.27 |
| 23 | CB | TYR | A | 339 | 34.765 | 63.638 | 15.915 | 1.00 | 50.52 |
| 24 | CG | TYR | A | 339 | 35.869 | 64.198 | 16.793 | 1.00 | 58.40 |
| 25 | CD1 | TYR | A | 339 | 37.144 | 64.445 | 16.274 | 1.00 | 61.23 |
| 26 | CD2 | TYR | A | 339 | 35.648 | 64.456 | 18.151 | 1.00 | 60.99 |
| 27 | CE1 | TYR | A | 339 | 38.174 | 64.929 | 17.081 | 1.00 | 62.71 |
| 28 | CE2 | TYR | A | 339 | 36.674 | 64.943 | 18.969 | 1.00 | 63.53 |
| 29 | CZ | TYR | A | 339 | 37.933 | 65.176 | 18.427 | 1.00 | 64.33 |
| 30 | OH | TYR | A | 339 | 38.953 | 65.644 | 19.230 | 1.00 | 65.07 |
| 31 | N | LEU | A | 340 | 32.793 | 61.291 | 14.746 | 1.00 | 41.90 |
| 32 | CA | LEU | A | 340 | 31.831 | 60.905 | 13.728 | 1.00 | 41.06 |
| 33 | C | LEU | A | 340 | 30.571 | 61.671 | 14.078 | 1.00 | 41.10 |
| 34 | O | LEU | A | 340 | 30.136 | 61.647 | 15.224 | 1.00 | 43.45 |
| 35 | CB | LEU | A | 340 | 31.583 | 59.392 | 13.779 | 1.00 | 36.93 |
| 36 | CG | LEU | A | 340 | 30.689 | 58.770 | 12.701 | 1.00 | 35.91 |
| 37 | CD1 | LEU | A | 340 | 31.229 | 59.100 | 11.314 | 1.00 | 34.60 |
| 38 | CD2 | LEU | A | 340 | 30.621 | 57.264 | 12.906 | 1.00 | 35.51 |
| 39 | N | SER | A | 341 | 29.990 | 62.368 | 13.108 | 1.00 | 39.82 |
| 40 | CA | SER | A | 341 | 28.790 | 63.152 | 13.385 | 1.00 | 39.85 |
| 41 | C | SER | A | 341 | 27.598 | 62.645 | 12.614 | 1.00 | 37.88 |
| 42 | O | SER | A | 341 | 27.737 | 62.076 | 11.544 | 1.00 | 42.10 |
| 43 | CB | SER | A | 341 | 29.025 | 64.627 | 13.042 | 1.00 | 39.34 |
| 44 | O | SER | A | 341 | 29.347 | 64.792 | 11.672 | 1.00 | 44.58 |
| 45 | N | ARG | A | 342 | 26.415 | 62.868 | 13.158 | 1.00 | 36.98 |
| 46 | CA | ARG | A | 342 | 25.187 | 62.437 | 12.517 | 1.00 | 35.20 |
| 47 | C | ARG | A | 342 | 24.853 | 63.417 | 11.393 | 1.00 | 33.18 |
| 48 | O | ARG | A | 342 | 25.508 | 64.443 | 11.252 | 1.00 | 33.16 |
| 49 | CB | ARG | A | 342 | 24.070 | 62.394 | 13.566 | 1.00 | 38.57 |
| 50 | CG | ARG | A | 342 | 24.321 | 61.381 | 14.689 | 1.00 | 39.10 |
| 51 | CD | ARG | A | 342 | 23.191 | 61.364 | 15.712 | 1.00 | 43.21 |
| 52 | NE | ARG | A | 342 | 23.231 | 62.544 | 16.570 | 1.00 | 45.40 |
| 53 | CZ | ARG | A | 342 | 24.086 | 62.714 | 17.573 | 1.00 | 48.22 |
| 54 | NH1 | ARG | A | 342 | 24.977 | 61.777 | 17.860 | 1.00 | 52.78 |
| 55 | NH2 | ARG | A | 342 | 24.059 | 63.831 | 18.286 | 1.00 | 54.34 |
| 56 | N | PRO | A | 343 | 23.843 | 63.112 | 10.570 | 1.00 | 33.03 |
| 57 | CA | PRO | A | 343 | 23.497 | 64.040 | 9.481 | 1.00 | 34.14 |
| 58 | C | PRO | A | 343 | 22.907 | 65.339 | 10.035 | 1.00 | 34.06 |
| 59 | O | PRO | A | 343 | 22.302 | 65.341 | 11.106 | 1.00 | 35.44 |
| 60 | CB | PRO | A | 343 | 22.448 | 63.266 | 8.667 | 1.00 | 33.07 |
| 61 | CG | PRO | A | 343 | 22.700 | 61.811 | 9.025 | 1.00 | 35.06 |
| 62 | CD | PRO | A | 343 | 23.029 | 61.885 | 10.499 | 1.00 | 33.27 |
| 63 | N | SER | A | 344 | 23.080 | 66.445 | 9.325 | 1.00 | 32.17 |
| 64 | CA | SER | A | 344 | 22.490 | 67.691 | 9.792 | 1.00 | 31.75 |
| 65 | C | SER | A | 344 | 21.014 | 67.617 | 9.414 | 1.00 | 32.07 |
| 66 | O | SER | A | 344 | 20.660 | 67.115 | 8.344 | 1.00 | 30.21 |
| 67 | CB | SER | A | 344 | 23.144 | 68.907 | 9.118 | 1.00 | 32.20 |
| 68 | OG | SER | A | 344 | 22.665 | 69.113 | 7.799 | 1.00 | 32.50 |
| 69 | N | PRO | A | 345 | 20.127 | 68.088 | 10.300 | 1.00 | 32.31 |
| 70 | CA | PRO | A | 345 | 18.701 | 68.039 | 9.975 | 1.00 | 30.84 |
| 71 | C | PRO | A | 345 | 18.381 | 68.710 | 8.629 | 1.00 | 29.64 |
| 72 | O | PRO | A | 345 | 17.506 | 68.256 | 7.891 | 1.00 | 30.14 |
| 73 | CB | PRO | A | 345 | 18.055 | 68.741 | 11.174 | 1.00 | 30.96 |
| 74 | CG | PRO | A | 345 | 18.941 | 68.301 | 12.307 | 1.00 | 33.18 |
| 75 | CD | PRO | A | 345 | 20.339 | 68.457 | 11.713 | 1.00 | 33.65 |
| 76 | N | PHE | A | 346 | 19.103 | 69.769 | 8.292 | 1.00 | 28.34 |
| 77 | CA | PHE | A | 346 | 18.844 | 70.447 | 7.034 | 1.00 | 31.46 |
| 78 | C | PHE | A | 346 | 19.085 | 69.522 | 5.831 | 1.00 | 32.46 |
| 79 | O | PHE | A | 346 | 18.269 | 69.465 | 4.907 | 1.00 | 32.59 |
| 80 | CB | PHE | A | 346 | 19.711 | 71.706 | 6.916 | 1.00 | 32.72 |
| 81 | CG | PHE | A | 346 | 19.613 | 72.382 | 5.579 | 1.00 | 36.34 |
| 82 | CD1 | PHE | A | 346 | 18.430 | 72.981 | 5.172 | 1.00 | 40.58 |
| 83 | CD2 | PHE | A | 346 | 20.702 | 72.411 | 4.722 | 1.00 | 36.76 |
| 84 | CE1 | PHE | A | 346 | 18.333 | 73.609 | 3.933 | 1.00 | 38.98 |
| 85 | CE2 | PHE | A | 346 | 20.615 | 73.037 | 3.482 | 1.00 | 40.15 |
| 86 | CZ | PHE | A | 346 | 19.425 | 73.637 | 3.086 | 1.00 | 38.60 |
| 87 | N | ASP | A | 347 | 20.203 | 68.797 | 5.850 | 1.00 | 32.92 |
| 88 | CA | ASP | A | 347 | 20.534 | 67.877 | 4.764 | 1.00 | 33.58 |
| 89 | C | ASP | A | 347 | 19.555 | 66.704 | 4.733 | 1.00 | 34.26 |
| 90 | O | ASP | A | 347 | 19.192 | 66.212 | 3.666 | 1.00 | 32.29 |
| 91 | CB | ASP | A | 347 | 21.967 | 67.349 | 4.921 | 1.00 | 28.72 |
| 92 | CG | ASP | A | 347 | 23.008 | 68.364 | 4.521 | 1.00 | 39.62 |
| 93 | OD1 | ASP | A | 347 | 24.225 | 68.085 | 4.671 | 1.00 | 42.08 |
| 94 | OD2 | ASP | A | 347 | 22.609 | 69.449 | 4.046 | 1.00 | 46.23 |
| 95 | N | LEU | A | 348 | 19.120 | 66.276 | 5.911 | 1.00 | 34.31 |
| 96 | CA | LEU | A | 348 | 18.197 | 65.159 | 6.036 | 1.00 | 37.70 |
| 97 | C | LEU | A | 348 | 16.742 | 65.441 | 5.629 | 1.00 | 38.98 |
| 98 | O | LEU | A | 348 | 16.137 | 64.651 | 4.902 | 1.00 | 38.11 |
| 99 | CB | LEU | A | 348 | 18.220 | 64.641 | 7.482 | 1.00 | 40.66 |
| 100 | CG | LEU | A | 348 | 17.333 | 63.441 | 7.840 | 1.00 | 41.99 |
| 101 | CD1 | LEU | A | 348 | 17.738 | 62.212 | 7.005 | 1.00 | 42.77 |
| 102 | CD2 | LEU | A | 348 | 17.476 | 63.131 | 9.341 | 1.00 | 41.00 |
| 103 | N | PHE | A | 349 | 16.183 | 66.560 | 6.083 | 1.00 | 39.68 |
| 104 | CA | PHE | A | 349 | 14.781 | 66.875 | 5.794 | 1.00 | 42.09 |
| 105 | C | PHE | A | 349 | 14.466 | 67.883 | 4.695 | 1.00 | 45.09 |
| 106 | O | PHE | A | 349 | 13.431 | 67.779 | 4.038 | 1.00 | 48.27 |
| 107 | CB | PHE | A | 349 | 14.074 | 67.328 | 7.071 | 1.00 | 37.36 |
| 108 | CG | PHE | A | 349 | 14.189 | 66.356 | 8.200 | 1.00 | 36.93 |
| 109 | CD1 | PHE | A | 349 | 15.021 | 66.624 | 9.282 | 1.00 | 38.16 |
| 110 | CD2 | PHE | A | 349 | 13.487 | 65.156 | 8.174 | 1.00 | 38.04 |
| 111 | CE1 | PHE | A | 349 | 15.155 | 65.715 | 10.319 | 1.00 | 38.11 |
| 112 | CE2 | PHE | A | 349 | 13.614 | 64.231 | 9.213 | 1.00 | 37.58 |
| 113 | CZ | PHE | A | 349 | 14.449 | 64.511 | 10.283 | 1.00 | 40.39 |
| 114 | N | ILE | A | 350 | 15.323 | 68.874 | 4.500 | 1.00 | 46.66 |
| 115 | CA | ILE | A | 350 | 15.052 | 69.854 | 3.461 | 1.00 | 48.94 |
| 116 | C | ILE | A | 350 | 15.695 | 69.400 | 2.150 | 1.00 | 51.15 |
| 117 | O | ILE | A | 350 | 15.007 | 69.095 | 1.179 | 1.00 | 50.51 |
| 118 | CB | ILE | A | 350 | 15.592 | 71.241 | 3.860 | 1.00 | 48.52 |
| 119 | CG1 | ILE | A | 350 | 15.172 | 71.562 | 5.299 | 1.00 | 49.25 |
| 120 | CG2 | ILE | A | 350 | 15.052 | 72.300 | 2.923 | 1.00 | 49.68 |
| 121 | CD1 | ILE | A | 350 | 13.689 | 71.358 | 5.567 | 1.00 | 46.80 |
| 122 | N | ARG | A | 351 | 17.021 | 69.335 | 2.155 | 1.00 | 52.64 |
| 123 | CA | ARG | A | 351 | 17.807 | 68.931 | 1.000 | 1.00 | 53.76 |
| 124 | C | ARG | A | 351 | 17.510 | 67.486 | 0.581 | 1.00 | 53.61 |
| 125 | O | ARG | A | 351 | 17.506 | 67.164 | −0.603 | 1.00 | 53.55 |
| 126 | CB | ARG | A | 351 | 19.287 | 69.095 | 1.341 | 1.00 | 56.64 |
| 127 | CG | ARG | A | 351 | 20.166 | 69.584 | 0.206 | 1.00 | 62.18 |
| 128 | CD | ARG | A | 351 | 21.225 | 70.527 | 0.752 | 1.00 | 66.96 |
| 129 | NE | ARG | A | 351 | 22.389 | 70.642 | −0.119 | 1.00 | 71.13 |
| 130 | CZ | ARG | A | 351 | 23.229 | 69.643 | −0.370 | 1.00 | 76.72 |
| 131 | NH1 | ARG | A | 351 | 23.027 | 68.455 | 0.184 | 1.00 | 78.73 |
| 132 | NH2 | ARG | A | 351 | 24.275 | 69.829 | −1.168 | 1.00 | 77.63 |
| 133 | N | LYS | A | 352 | 17.253 | 66.626 | 1.560 | 1.00 | 53.86 |
| 134 | CA | LYS | A | 352 | 16.954 | 65.215 | 1.312 | 1.00 | 54.12 |
| 135 | C | LYS | A | 352 | 18.151 | 64.428 | 0.772 | 1.00 | 52.02 |
| 136 | O | LYS | A | 352 | 17.996 | 63.522 | −0.046 | 1.00 | 51.77 |
| 137 | CB | LYS | A | 352 | 15.775 | 65.075 | 0.347 | 1.00 | 57.77 |
| 138 | CG | LYS | A | 352 | 14.476 | 65.689 | 0.846 | 1.00 | 62.15 |
| 139 | CD | LYS | A | 352 | 13.348 | 65.439 | −0.154 | 1.00 | 71.06 |
| 140 | CE | LYS | A | 352 | 12.098 | 66.249 | 0.171 | 1.00 | 74.25 |
| 141 | NZ | LYS | A | 352 | 12.293 | 67.707 | −0.085 | 1.00 | 78.35 |
| 142 | N | SER | A | 353 | 19.342 | 64.789 | 1.233 | 1.00 | 48.01 |
| 143 | CA | SER | A | 353 | 20.568 | 64.112 | 0.832 | 1.00 | 45.29 |
| 144 | C | SER | A | 353 | 21.544 | 64.154 | 2.016 | 1.00 | 40.67 |
| 145 | O | SER | A | 353 | 22.546 | 64.875 | 1.994 | 1.00 | 40.76 |
| 146 | CB | SER | A | 353 | 21.177 | 64.791 | −0.398 | 1.00 | 47.97 |
| 147 | OG | SER | A | 353 | 21.448 | 66.155 | −0.147 | 1.00 | 56.20 |
| 148 | N | PRO | A | 354 | 21.244 | 63.375 | 3.070 | 1.00 | 36.53 |
| 149 | CA | PRO | A | 354 | 22.004 | 63.239 | 4.319 | 1.00 | 32.68 |
| 150 | C | PRO | A | 354 | 23.399 | 62.660 | 4.139 | 1.00 | 31.88 |
| 151 | O | PRO | A | 354 | 23.615 | 61.787 | 3.302 | 1.00 | 29.76 |
| 152 | CB | PRO | A | 354 | 21.147 | 62.294 | 5.160 | 1.00 | 30.29 |
| 153 | CG | PRO | A | 354 | 19.799 | 62.340 | 4.513 | 1.00 | 41.68 |
| 154 | CD | PRO | A | 354 | 20.106 | 62.445 | 3.065 | 1.00 | 33.29 |
| 155 | N | THR | A | 355 | 24.342 | 63.154 | 4.932 | 1.00 | 30.03 |
| 156 | CA | THR | A | 355 | 25.704 | 62.644 | 4.902 | 1.00 | 29.70 |
| 157 | C | THR | A | 355 | 26.236 | 62.578 | 6.326 | 1.00 | 30.82 |
| 158 | O | THR | A | 355 | 25.773 | 63.298 | 7.213 | 1.00 | 30.67 |
| 159 | CE | THR | A | 355 | 26.669 | 63.558 | 4.136 | 1.00 | 32.14 |
| 160 | OG1 | THR | A | 355 | 26.806 | 64.785 | 4.860 | 1.00 | 29.97 |
| 161 | CG2 | THR | A | 355 | 26.175 | 63.837 | 2.707 | 1.00 | 31.76 |
| 162 | N | ILE | A | 356 | 27.195 | 61.693 | 6.558 | 1.00 | 30.53 |
| 163 | CA | ILE | A | 356 | 27.816 | 61.631 | 7.868 | 1.00 | 31.56 |
| 164 | C | ILE | A | 356 | 29.279 | 61.860 | 7.575 | 1.00 | 31.79 |
| 165 | O | ILE | A | 356 | 29.752 | 61.561 | 6.475 | 1.00 | 30.35 |
| 166 | CB | ILE | A | 356 | 27.584 | 60.285 | 8.583 | 1.00 | 30.05 |
| 167 | CG1 | ILE | A | 356 | 28.101 | 59.133 | 7.729 | 1.00 | 31.32 |
| 168 | CG2 | ILE | A | 356 | 26.092 | 60.131 | 8.890 | 1.00 | 31.27 |
| 169 | CD1 | ILE | A | 356 | 27.843 | 57.772 | 8.337 | 1.00 | 36.89 |
| 170 | N | THR | A | 357 | 29.995 | 62.402 | 8.550 | 1.00 | 33.31 |
| 171 | CA | THR | A | 357 | 31.395 | 62.719 | 8.349 | 1.00 | 33.68 |
| 172 | C | THR | A | 357 | 32.282 | 62.197 | 9.456 | 1.00 | 35.47 |
| 173 | O | THR | A | 357 | 31.993 | 62.372 | 10.641 | 1.00 | 36.65 |
| 174 | CB | THR | A | 357 | 31.589 | 64.265 | 8.245 | 1.00 | 33.81 |
| 175 | OG1 | THR | A | 357 | 30.917 | 64.756 | 7.077 | 1.00 | 33.71 |
| 176 | CG2 | THR | A | 357 | 33.065 | 64.632 | 8.179 | 1.00 | 29.63 |
| 177 | N | CYS | A | 358 | 33.372 | 61.566 | 9.052 | 1.00 | 35.41 |
| 178 | CA | CYS | A | 358 | 34.346 | 61.039 | 9.990 | 1.00 | 38.12 |
| 179 | C | CYS | A | 358 | 35.507 | 62.030 | 9.935 | 1.00 | 37.77 |
| 180 | O | CYS | A | 358 | 36.127 | 62.205 | 8.890 | 1.00 | 36.67 |
| 181 | CB | CYS | A | 358 | 34.811 | 59.655 | 9.532 | 1.00 | 40.37 |
| 182 | SG | CYS | A | 358 | 35.831 | 58.729 | 10.726 | 1.00 | 49.83 |
| 183 | N | LEU | A | 359 | 35.778 | 62.691 | 11.054 | 1.00 | 39.77 |
| 184 | CA | LEU | A | 359 | 36.853 | 63.668 | 11.127 | 1.00 | 42.86 |
| 185 | C | LEU | A | 359 | 38.007 | 63.168 | 11.977 | 1.00 | 46.01 |
| 186 | O | LEU | A | 359 | 37.831 | 62.852 | 13.152 | 1.00 | 47.43 |
| 187 | CB | LEU | A | 359 | 36.335 | 64.988 | 11.711 | 1.00 | 42.74 |
| 188 | CG | LEU | A | 359 | 37.392 | 65.996 | 12.196 | 1.00 | 45.03 |
| 189 | CD1 | LEU | A | 359 | 38.262 | 66.448 | 11.043 | 1.00 | 47.59 |
| 190 | CD2 | LEU | A | 359 | 36.711 | 67.197 | 12.838 | 1.00 | 47.51 |
| 191 | N | VAL | A | 360 | 39.189 | 63.107 | 11.379 | 1.00 | 48.26 |
| 192 | CA | VAL | A | 360 | 40.380 | 62.669 | 12.090 | 1.00 | 52.34 |
| 193 | C | VAL | A | 360 | 41.357 | 63.835 | 12.217 | 1.00 | 55.48 |
| 194 | O | VAL | A | 360 | 41.672 | 64.508 | 11.234 | 1.00 | 55.57 |
| 195 | CB | VAL | A | 360 | 41.073 | 61.500 | 11.353 | 1.00 | 53.36 |
| 196 | CG1 | VAL | A | 360 | 42.360 | 61.119 | 12.071 | 1.00 | 51.62 |
| 197 | CG2 | VAL | A | 360 | 40.133 | 60.301 | 11.284 | 1.00 | 51.77 |
| 198 | N | VAL | A | 361 | 41.827 | 64.080 | 13.435 | 1.00 | 58.92 |
| 199 | CA | VAL | A | 361 | 42.766 | 65.167 | 13.684 | 1.00 | 63.13 |
| 200 | C | VAL | A | 361 | 44.094 | 64.607 | 14.174 | 1.00 | 67.77 |
| 201 | O | VAL | A | 361 | 44.134 | 63.862 | 15.154 | 1.00 | 68.38 |
| 202 | CB | VAL | A | 361 | 42.223 | 66.158 | 14.747 | 1.00 | 60.50 |
| 203 | CG1 | VAL | A | 361 | 43.245 | 67.242 | 15.014 | 1.00 | 56.92 |
| 204 | CG2 | VAL | A | 361 | 40.929 | 66.786 | 14.270 | 1.00 | 56.10 |
| 205 | N | ASP | A | 362 | 45.174 | 64.965 | 13.485 | 1.00 | 72.90 |
| 206 | CA | ASP | A | 362 | 46.515 | 64.502 | 13.841 | 1.00 | 78.30 |
| 207 | C | ASP | A | 362 | 47.356 | 65.696 | 14.284 | 1.00 | 81.68 |
| 208 | O | ASP | A | 362 | 47.891 | 66.432 | 13.452 | 1.00 | 81.72 |
| 209 | CB | ASP | A | 362 | 47.185 | 63.829 | 12.638 | 1.00 | 80.22 |
| 210 | CG | ASP | A | 362 | 48.444 | 63.070 | 13.020 | 1.00 | 84.67 |
| 211 | OD1 | ASP | A | 362 | 49.122 | 63.487 | 13.983 | 1.00 | 87.54 |
| 212 | OD2 | ASP | A | 362 | 48.763 | 62.063 | 12.351 | 1.00 | 87.19 |
| 213 | N | LEU | A | 363 | 47.474 | 65.877 | 15.597 | 1.00 | 85.84 |
| 214 | CA | LEU | A | 363 | 48.234 | 66.986 | 16.165 | 1.00 | 89.95 |
| 215 | C | LEU | A | 363 | 49.677 | 67.038 | 15.672 | 1.00 | 92.20 |
| 216 | O | LEU | A | 363 | 50.214 | 68.117 | 15.432 | 1.00 | 92.55 |
| 217 | CB | LEU | A | 363 | 48.210 | 66.906 | 17.691 | 1.00 | 91.66 |
| 218 | CG | LEU | A | 363 | 46.823 | 66.881 | 18.342 | 1.00 | 92.93 |
| 219 | CD1 | LEU | A | 363 | 46.976 | 66.787 | 19.854 | 1.00 | 95.41 |
| 220 | CD2 | LEU | A | 363 | 46.049 | 68.129 | 17.963 | 1.00 | 92.73 |
| 221 | N | ALA | A | 364 | 50.304 | 65.874 | 15.527 | 1.00 | 94.76 |
| 222 | CA | ALA | A | 364 | 51.684 | 65.799 | 15.049 | 1.00 | 97.66 |
| 223 | C | ALA | A | 364 | 51.769 | 64.806 | 13.890 | 1.00 | 99.72 |
| 224 | O | ALA | A | 364 | 51.604 | 63.602 | 14.079 | 1.00 | 100.44 |
| 225 | GB | ALA | A | 364 | 52.604 | 65.365 | 16.179 | 1.00 | 97.99 |
| 226 | N | PRO | A | 365 | 52.023 | 65.303 | 12.671 | 1.00 | 101.15 |
| 227 | CA | PRO | A | 365 | 52.116 | 64.427 | 11.498 | 1.00 | 102.25 |
| 228 | C | PRO | A | 365 | 53.437 | 63.705 | 11.245 | 1.00 | 103.43 |
| 229 | O | PRO | A | 365 | 54.497 | 64.326 | 11.166 | 1.00 | 104.19 |
| 230 | CB | PRO | A | 365 | 51.771 | 65.363 | 10.339 | 1.00 | 101.91 |
| 231 | CG | PRO | A | 365 | 50.983 | 66.479 | 11.002 | 1.00 | 102.27 |
| 232 | CD | PRO | A | 365 | 51.767 | 66.690 | 12.254 | 1.00 | 101.40 |
| 233 | N | SER | A | 366 | 53.344 | 62.385 | 11.121 | 1.00 | 104.18 |
| 234 | CA | SER | A | 366 | 54.468 | 61.512 | 10.801 | 1.00 | 104.43 |
| 235 | C | SER | A | 366 | 53.924 | 61.027 | 9.463 | 1.00 | 104.05 |
| 236 | O | SER | A | 366 | 52.736 | 60.712 | 9.373 | 1.00 | 104.36 |
| 237 | CB | SER | A | 366 | 54.545 | 60.351 | 11.788 | 1.00 | 105.50 |
| 238 | OG | SER | A | 366 | 53.377 | 59.560 | 11.687 | 1.00 | 106.70 |
| 239 | N | LYS | A | 367 | 54.739 | 60.955 | 8.418 | 1.00 | 103.19 |
| 240 | CA | LYS | A | 367 | 54.138 | 60.559 | 7.156 | 1.00 | 102.08 |
| 241 | C | LYS | A | 367 | 53.970 | 59.109 | 6.733 | 1.00 | 100.74 |
| 242 | O | LYS | A | 367 | 54.917 | 58.366 | 6.460 | 1.00 | 101.05 |
| 243 | CB | LYS | A | 367 | 54.732 | 61.364 | 5.998 | 1.00 | 103.30 |
| 244 | CG | LYS | A | 367 | 56.216 | 61.257 | 5.755 | 1.00 | 105.00 |
| 245 | CD | LYS | A | 367 | 56.554 | 62.226 | 4.633 | 1.00 | 104.80 |
| 246 | CE | LYS | A | 367 | 57.990 | 62.133 | 4.170 | 1.00 | 104.70 |
| 247 | NZ | LYS | A | 367 | 58.214 | 63.076 | 3.034 | 1.00 | 105.18 |
| 248 | N | GLY | A | 368 | 52.691 | 58.761 | 6.678 | 1.00 | 98.70 |
| 249 | CA | GLY | A | 368 | 52.192 | 57.464 | 6.272 | 1.00 | 95.62 |
| 250 | C | GLY | A | 368 | 50.799 | 57.900 | 5.856 | 1.00 | 93.12 |
| 251 | O | GLY | A | 368 | 50.457 | 59.062 | 6.065 | 1.00 | 93.17 |
| 252 | N | THR | A | 369 | 49.983 | 57.031 | 5.277 | 1.00 | 90.15 |
| 253 | CA | THR | A | 369 | 48.652 | 57.475 | 4.880 | 1.00 | 86.41 |
| 254 | C | THR | A | 369 | 47.623 | 57.180 | 5.964 | 1.00 | 83.04 |
| 255 | O | THR | A | 369 | 47.746 | 56.198 | 6.699 | 1.00 | 82.54 |
| 256 | CB | THR | A | 369 | 48.203 | 56.808 | 3.559 | 1.00 | 87.73 |
| 257 | OG1 | THR | A | 369 | 48.178 | 55.383 | 3.721 | 1.00 | 88.60 |
| 258 | CG2 | THR | A | 369 | 49.157 | 57.177 | 2.429 | 1.00 | 86.64 |
| 259 | N | VAL | A | 370 | 46.620 | 58.048 | 6.073 | 1.00 | 79.07 |
| 260 | CA | VAL | A | 370 | 45.559 | 57.871 | 7.058 | 1.00 | 74.46 |
| 261 | C | VAL | A | 370 | 44.339 | 57.325 | 6.332 | 1.00 | 72.21 |
| 262 | O | VAL | A | 370 | 43.762 | 57.998 | 5.482 | 1.00 | 71.35 |
| 263 | CB | VAL | A | 370 | 45.180 | 59.199 | 7.732 | 1.00 | 73.33 |
| 264 | CG1 | VAL | A | 370 | 44.254 | 58.933 | 8.904 | 1.00 | 70.92 |
| 265 | CG2 | VAL | A | 370 | 46.427 | 59.926 | 8.185 | 1.00 | 69.77 |
| 266 | N | ASN | A | 371 | 43.947 | 56.102 | 6.674 | 1.00 | 70.13 |
| 267 | CA | ASN | A | 371 | 42.809 | 55.463 | 6.027 | 1.00 | 68.15 |
| 268 | C | ASN | A | 371 | 41.521 | 55.443 | 6.838 | 1.00 | 65.65 |
| 269 | O | ASN | A | 371 | 41.533 | 55.248 | 8.054 | 1.00 | 64.64 |
| 270 | CB | ASN | A | 371 | 43.179 | 54.036 | 5.631 | 1.00 | 70.12 |
| 271 | CG | ASN | A | 371 | 44.270 | 53.995 | 4.592 | 1.00 | 73.56 |
| 272 | OD1 | ASN | A | 371 | 44.081 | 54.447 | 3.462 | 1.00 | 75.10 |
| 273 | ND2 | ASN | A | 371 | 45.427 | 53.463 | 4.969 | 1.00 | 74.76 |
| 274 | N | LEU | A | 372 | 40.408 | 55.651 | 6.141 | 1.00 | 62.66 |
| 275 | CA | LEU | A | 372 | 39.088 | 55.641 | 6.754 | 1.00 | 59.53 |
| 276 | C | LEU | A | 372 | 38.226 | 54.673 | 5.963 | 1.00 | 56.68 |
| 277 | O | LEU | A | 372 | 38.071 | 54.812 | 4.753 | 1.00 | 56.70 |
| 278 | CB | LEU | A | 372 | 38.459 | 57.040 | 6.728 | 1.00 | 59.44 |
| 279 | CG | LEU | A | 372 | 39.217 | 58.172 | 7.422 | 1.00 | 60.55 |
| 280 | CD1 | LEU | A | 372 | 38.323 | 59.386 | 7.521 | 1.00 | 61.28 |
| 281 | CD2 | LEU | A | 372 | 39.644 | 57.739 | 8.807 | 1.00 | 62.83 |
| 282 | N | THR | A | 373 | 37.669 | 53.689 | 6.654 | 1.00 | 53.76 |
| 283 | CA | THR | A | 373 | 36.834 | 52.685 | 6.015 | 1.00 | 51.64 |
| 284 | C | THR | A | 373 | 35.420 | 52.750 | 6.588 | 1.00 | 48.20 |
| 285 | O | THR | A | 373 | 35.245 | 52.799 | 7.804 | 1.00 | 46.65 |
| 286 | CB | THR | A | 373 | 37.399 | 51.275 | 6.268 | 1.00 | 53.15 |
| 287 | OG1 | THR | A | 373 | 38.811 | 51.276 | 6.027 | 1.00 | 57.77 |
| 288 | CG2 | THR | A | 373 | 36.729 | 50.261 | 5.353 | 1.00 | 53.75 |
| 289 | N | THR | A | 374 | 34.419 | 52.749 | 5.715 | 1.00 | 45.34 |
| 290 | CA | THR | A | 374 | 33.025 | 52.801 | 6.158 | 1.00 | 44.94 |
| 291 | C | THR | A | 374 | 32.341 | 51.439 | 6.059 | 1.00 | 43.68 |
| 292 | O | THR | A | 374 | 32.636 | 50.655 | 5.162 | 1.00 | 42.67 |
| 293 | CB | THR | A | 374 | 32.209 | 53.795 | 5.316 | 1.00 | 41.63 |
| 294 | CG | THR | A | 374 | 32.623 | 55.229 | 5.443 | 1.00 | 41.50 |
| 295 | CD1 | THR | A | 374 | 33.476 | 55.912 | 4.623 | 1.00 | 39.67 |
| 296 | CD2 | THR | A | 374 | 32.205 | 56.159 | 6.452 | 1.00 | 35.69 |
| 297 | NE1 | TEP | A | 374 | 33.612 | 57.208 | 5.059 | 1.00 | 35.06 |
| 298 | CE2 | THR | A | 374 | 32.844 | 57.386 | 6.178 | 1.00 | 34.53 |
| 299 | CE3 | THR | A | 374 | 31.353 | 56.072 | 7.558 | 1.00 | 32.37 |
| 300 | CZ2 | THR | A | 374 | 32.657 | 58.521 | 6.971 | 1.00 | 35.43 |
| 301 | CZ3 | THR | A | 374 | 31.166 | 57.199 | 8.349 | 1.00 | 36.11 |
| 302 | CH2 | THR | A | 374 | 31.818 | 58.410 | 8.050 | 1.00 | 37.51 |
| 303 | N | SER | A | 375 | 31.421 | 51.170 | 6.981 | 1.00 | 43.53 |
| 304 | CA | SER | A | 375 | 30.666 | 49.922 | 6.960 | 1.00 | 43.57 |
| 305 | C | SER | A | 375 | 29.330 | 50.089 | 7.662 | 1.00 | 42.59 |
| 306 | O | SER | A | 375 | 29.189 | 50.931 | 8.550 | 1.00 | 43.68 |
| 307 | CB | SER | A | 375 | 31.447 | 48.780 | 7.626 | 1.00 | 45.03 |
| 308 | OG | SER | A | 375 | 31.482 | 48.909 | 9.034 | 1.00 | 45.66 |
| 309 | N | ARG | A | 376 | 28.349 | 49.301 | 7.234 | 1.00 | 40.79 |
| 310 | CA | ARG | A | 376 | 27.031 | 49.321 | 7.834 | 1.00 | 40.12 |
| 311 | C | ARG | A | 376 | 26.911 | 48.102 | 8.749 | 1.00 | 40.59 |
| 312 | O | ARG | A | 376 | 27.207 | 46.982 | 8.348 | 1.00 | 40.90 |
| 313 | CB | ARG | A | 376 | 25.947 | 49.255 | 6.766 | 1.00 | 40.03 |
| 314 | CG | ARG | A | 376 | 25.855 | 50.468 | 5.863 | 1.00 | 43.20 |
| 315 | CD | ARG | A | 376 | 24.402 | 50.717 | 5.501 | 1.00 | 41.05 |
| 316 | NE | ARG | A | 376 | 24.120 | 50.429 | 4.108 | 1.00 | 50.71 |
| 317 | CZ | ARG | A | 376 | 22.895 | 50.300 | 3.612 | 1.00 | 53.97 |
| 318 | NH1 | ARG | A | 376 | 21.842 | 50.428 | 4.407 | 1.00 | 51.88 |
| 319 | NH2 | ARG | A | 376 | 22.726 | 50.058 | 2.318 | 1.00 | 55.55 |
| 320 | N | ALA | A | 377 | 26.471 | 48.319 | 9.978 | 1.00 | 40.20 |
| 321 | CA | ALA | A | 377 | 26.327 | 47.218 | 10.919 | 1.00 | 40.25 |
| 322 | C | ALA | A | 377 | 25.470 | 46.100 | 10.314 | 1.00 | 40.38 |
| 323 | O | ALA | A | 377 | 25.621 | 44.943 | 10.678 | 1.00 | 41.16 |
| 324 | CB | ALA | A | 377 | 25.697 | 47.721 | 12.222 | 1.00 | 33.12 |
| 325 | N | SER | A | 378 | 24.585 | 46.456 | 9.386 | 1.00 | 40.21 |
| 326 | CA | SER | A | 378 | 23.697 | 45.491 | 8.746 | 1.00 | 41.95 |
| 327 | C | SER | A | 378 | 24.412 | 44.694 | 7.664 | 1.00 | 44.92 |
| 328 | O | SER | A | 378 | 23.856 | 43.734 | 7.134 | 1.00 | 44.28 |
| 329 | CB | SER | A | 378 | 22.504 | 46.199 | 8.108 | 1.00 | 40.91 |
| 330 | OG | SER | A | 378 | 22.907 | 46.863 | 6.920 | 1.00 | 40.09 |
| 331 | N | GLY | A | 379 | 25.633 | 45.107 | 7.332 | 1.00 | 46.93 |
| 332 | CA | GLY | A | 379 | 26.405 | 44.419 | 6.313 | 1.00 | 49.77 |
| 333 | C | GLY | A | 379 | 26.088 | 44.884 | 4.904 | 1.00 | 50.60 |
| 334 | O | GLY | A | 379 | 26.817 | 44.576 | 3.958 | 1.00 | 52.95 |
| 335 | N | LYS | A | 380 | 24.995 | 45.626 | 4.755 | 1.00 | 53.72 |
| 336 | CA | LYS | A | 380 | 24.612 | 46.122 | 3.443 | 1.00 | 54.13 |
| 337 | C | LYS | A | 380 | 25.689 | 47.036 | 2.845 | 1.00 | 48.16 |
| 338 | O | LYS | A | 380 | 26.592 | 47.513 | 3.544 | 1.00 | 45.65 |
| 339 | CB | LYS | A | 380 | 23.262 | 46.848 | 3.517 | 1.00 | 62.44 |
| 340 | CG | LYS | A | 380 | 22.078 | 45.908 | 3.710 | 1.00 | 67.72 |
| 341 | CD | LYS | A | 380 | 20.748 | 46.625 | 3.550 | 1.00 | 69.32 |
| 342 | CE | LYS | A | 380 | 19.587 | 45.644 | 3.619 | 1.00 | 73.14 |
| 343 | NZ | LYS | A | 380 | 18.267 | 46.320 | 3.467 | 1.00 | 77.37 |
| 344 | N | PRO | A | 381 | 25.614 | 47.277 | 1.529 | 1.00 | 56.07 |
| 345 | CA | PRO | A | 381 | 26.586 | 48.128 | 0.835 | 1.00 | 56.46 |
| 346 | C | PRO | A | 381 | 26.535 | 49.607 | 1.225 | 1.00 | 54.32 |
| 347 | O | PRO | A | 381 | 25.467 | 50.154 | 1.511 | 1.00 | 56.76 |
| 348 | CB | PRO | A | 381 | 26.225 | 47.936 | −0.640 | 1.00 | 60.70 |
| 349 | CG | PRO | A | 381 | 25.481 | 46.622 | −0.667 | 1.00 | 62.01 |
| 350 | CB | PRO | A | 381 | 24.655 | 46.695 | 0.574 | 1.00 | 59.24 |
| 351 | N | VAL | A | 382 | 27.701 | 50.244 | 1.234 | 1.00 | 55.74 |
| 352 | CA | VAL | A | 382 | 27.795 | 51.669 | 1.531 | 1.00 | 55.58 |
| 353 | C | VAL | A | 382 | 28.192 | 52.353 | 0.220 | 1.00 | 55.56 |
| 354 | O | VAL | A | 382 | 28.866 | 51.752 | −0.620 | 1.00 | 55.22 |
| 355 | CB | VAL | A | 382 | 28.874 | 51.980 | 2.607 | 1.00 | 55.39 |
| 356 | CG1 | VAL | A | 382 | 28.592 | 51.196 | 3.878 | 1.00 | 55.94 |
| 357 | CG2 | VAL | A | 382 | 30.261 | 51.664 | 2.075 | 1.00 | 53.06 |
| 358 | N | ASN | A | 383 | 27.773 | 53.600 | 0.045 | 1.00 | 54.38 |
| 359 | CA | ASN | A | 383 | 28.090 | 54.354 | −1.164 | 1.00 | 54.63 |
| 360 | C | ASN | A | 383 | 29.532 | 54.843 | −1.190 | 1.00 | 53.85 |
| 361 | O | ASN | A | 383 | 30.303 | 54.630 | −0.254 | 1.00 | 52.58 |
| 362 | CB | ASN | A | 383 | 27.171 | 55.566 | −1.282 | 1.00 | 59.07 |
| 363 | CG | ASN | A | 383 | 25.717 | 55.186 | −1.356 | 1.00 | 64.60 |
| 364 | OD1 | ASN | A | 383 | 24.836 | 56.024 | −1.169 | 1.00 | 70.14 |
| 365 | ND2 | ASN | A | 383 | 25.450 | 53.915 | −1.638 | 1.00 | 71.07 |
| 366 | N | HIS | A | 384 | 29.890 | 55.506 | −2.281 | 1.00 | 53.46 |
| 367 | CA | HIS | A | 384 | 31.229 | 56.057 | −2.426 | 1.00 | 52.22 |
| 368 | C | HIS | A | 384 | 31.326 | 57.302 | −1.540 | 1.00 | 49.10 |
| 369 | O | HIS | A | 384 | 30.400 | 58.105 | −1.485 | 1.00 | 45.53 |
| 370 | CB | HIS | A | 384 | 31.489 | 56.417 | −3.893 | 1.00 | 59.11 |
| 371 | CG | HIS | A | 384 | 31.676 | 55.223 | −4.778 | 1.00 | 62.16 |
| 372 | ND1 | HIS | A | 384 | 32.738 | 54.357 | −4.639 | 1.00 | 69.15 |
| 373 | CB2 | HIS | A | 384 | 30.931 | 54.745 | −5.803 | 1.00 | 70.28 |
| 374 | CE1 | HIS | A | 384 | 32.640 | 53.394 | −5.539 | 1.00 | 75.01 |
| 375 | NE2 | HIS | A | 384 | 31.553 | 53.605 | −6.258 | 1.00 | 74.58 |
| 376 | N | SER | A | 385 | 32.448 | 57.454 | −0.849 | 1.00 | 45.07 |
| 377 | CA | SER | A | 385 | 32.641 | 58.583 | 0.040 | 1.00 | 44.61 |
| 378 | C | SER | A | 385 | 33.617 | 59.627 | −0.504 | 1.00 | 44.58 |
| 379 | O | SER | A | 385 | 34.380 | 59.356 | −1.421 | 1.00 | 44.21 |
| 380 | CB | SER | A | 385 | 33.113 | 58.076 | 1.409 | 1.00 | 44.61 |
| 381 | OG | SER | A | 385 | 34.293 | 57.290 | 1.311 | 1.00 | 41.13 |
| 382 | N | THR | A | 386 | 33.575 | 60.827 | 0.065 | 1.00 | 44.32 |
| 383 | CA | THR | A | 386 | 34.450 | 61.916 | −0.346 | 1.00 | 44.35 |
| 384 | C | THR | A | 386 | 35.487 | 62.156 | 0.737 | 1.00 | 44.09 |
| 385 | O | THR | A | 386 | 35.149 | 62.326 | 1.911 | 1.00 | 44.06 |
| 386 | CB | THR | A | 386 | 33.664 | 63.226 | −0.580 | 1.00 | 45.20 |
| 387 | OG1 | THR | A | 386 | 32.744 | 63.045 | −1.662 | 1.00 | 51.89 |
| 388 | CG2 | THR | A | 386 | 34.616 | 64.368 | −0.936 | 1.00 | 47.20 |
| 389 | N | ARG | A | 387 | 36.749 | 62.176 | 0.326 | 1.00 | 44.39 |
| 390 | CA | ARG | A | 387 | 37.870 | 62.378 | 1.233 | 1.00 | 46.68 |
| 391 | C | ARG | A | 387 | 38.520 | 63.763 | 1.070 | 1.00 | 47.05 |
| 392 | O | ARG | A | 387 | 38.750 | 64.228 | −0.049 | 1.00 | 46.65 |
| 393 | CB | ARG | A | 387 | 38.906 | 61.284 | 0.979 | 1.00 | 48.13 |
| 394 | CG | ARG | A | 387 | 40.112 | 61.330 | 1.890 | 1.00 | 52.77 |
| 395 | CD | ARG | A | 387 | 41.247 | 60.494 | 1.320 | 1.00 | 56.60 |
| 396 | NE | ARG | A | 387 | 42.459 | 60.622 | 2.121 | 1.00 | 60.65 |
| 397 | CZ | ARG | A | 387 | 42.705 | 59.917 | 3.218 | 1.00 | 61.97 |
| 398 | NH1 | ARG | A | 387 | 41.821 | 59.022 | 3.636 | 1.00 | 64.45 |
| 399 | NH2 | ARG | A | 387 | 43.819 | 60.131 | 3.911 | 1.00 | 60.54 |
| 400 | N | LYS | A | 388 | 38.820 | 64.415 | 2.190 | 1.00 | 46.93 |
| 401 | CA | LYS | A | 388 | 39.457 | 65.730 | 2.167 | 1.00 | 47.51 |
| 402 | C | LYS | A | 388 | 40.630 | 65.777 | 3.143 | 1.00 | 48.33 |
| 403 | O | LYS | A | 388 | 40.493 | 65.401 | 4.306 | 1.00 | 46.08 |
| 404 | CB | LYS | A | 388 | 38.452 | 66.823 | 2.537 | 1.00 | 46.64 |
| 405 | CG | LYS | A | 388 | 37.274 | 66.909 | 1.603 | 1.00 | 52.75 |
| 406 | CD | LYS | A | 388 | 36.154 | 67.739 | 2.195 | 1.00 | 58.62 |
| 407 | CE | LYS | A | 388 | 34.863 | 67.538 | 1.418 | 1.00 | 62.49 |
| 408 | NZ | LYS | A | 388 | 33.719 | 68.204 | 2.101 | 1.00 | 67.33 |
| 409 | N | GLU | A | 389 | 41.780 | 66.236 | 2.651 | 1.00 | 50.07 |
| 410 | CA | GLU | A | 389 | 42.998 | 66.357 | 3.454 | 1.00 | 51.86 |
| 411 | C | GLU | A | 389 | 43.474 | 67.807 | 3.462 | 1.00 | 52.81 |
| 412 | O | GLU | A | 389 | 43.683 | 68.408 | 2.405 | 1.00 | 49.21 |
| 413 | CB | GLU | A | 389 | 44.106 | 65.474 | 2.877 | 1.00 | 54.67 |
| 414 | CG | GLU | A | 389 | 43.890 | 63.984 | 3.056 | 1.00 | 65.65 |
| 415 | CD | GLU | A | 389 | 44.992 | 63.159 | 2.412 | 1.00 | 72.66 |
| 416 | OE1 | GLU | A | 389 | 46.182 | 63.449 | 2.671 | 1.00 | 75.92 |
| 417 | OE2 | GLU | A | 389 | 44.669 | 62.220 | 1.650 | 1.00 | 75.78 |
| 418 | N | GLU | A | 390 | 43.652 | 68.371 | 4.651 | 1.00 | 56.06 |
| 419 | CA | GLU | A | 390 | 44.100 | 69.751 | 4.754 | 1.00 | 61.17 |
| 420 | C | GLU | A | 390 | 45.146 | 69.971 | 5.835 | 1.00 | 64.70 |
| 421 | O | GLU | A | 390 | 45.002 | 69.528 | 6.977 | 1.00 | 64.01 |
| 422 | CB | GLU | A | 390 | 42.908 | 70.673 | 4.993 | 1.00 | 64.35 |
| 423 | CG | GLU | A | 390 | 43.283 | 72.121 | 5.233 | 1.00 | 74.80 |
| 424 | CD | GLU | A | 390 | 42.091 | 73.052 | 5.121 | 1.00 | 85.97 |
| 425 | OE1 | GLU | A | 390 | 41.053 | 72.778 | 5.764 | 1.00 | 92.60 |
| 426 | OE2 | GLU | A | 390 | 42.197 | 74.059 | 4.388 | 1.00 | 92.18 |
| 427 | N | LYS | A | 391 | 46.204 | 70.679 | 5.461 | 1.00 | 68.18 |
| 428 | CA | LYS | A | 391 | 47.288 | 70.960 | 6.388 | 1.00 | 71.58 |
| 429 | C | LYS | A | 391 | 47.298 | 72.385 | 6.920 | 1.00 | 76.11 |
| 430 | O | LYS | A | 391 | 46.927 | 73.335 | 6.231 | 1.00 | 75.89 |
| 431 | CB | LYS | A | 391 | 48.627 | 70.654 | 5.728 | 1.00 | 67.28 |
| 432 | N | GLN | A | 392 | 47.724 | 72.508 | 8.168 | 1.00 | 81.85 |
| 433 | CA | GLN | A | 392 | 47.854 | 73.786 | 8.846 | 1.00 | 88.38 |
| 434 | C | GLN | A | 392 | 49.118 | 73.574 | 9.657 | 1.00 | 92.30 |
| 435 | O | GLN | A | 392 | 49.100 | 72.848 | 10.648 | 1.00 | 93.25 |
| 436 | CB | GLN | A | 392 | 46.662 | 74.045 | 9.777 | 1.00 | 85.87 |
| 437 | CG | GLN | A | 392 | 45.752 | 75.178 | 9.318 | 1.00 | 90.76 |
| 438 | CB | GLN | A | 392 | 44.727 | 75.573 | 10.369 | 1.00 | 93.39 |
| 439 | OE1 | GLN | A | 392 | 45.076 | 75.913 | 11.502 | 1.00 | 92.93 |
| 440 | NE2 | GLN | A | 392 | 43.453 | 75.538 | 9.993 | 1.00 | 93.97 |
| 441 | N | ARG | A | 393 | 50.221 | 74.174 | 9.220 | 1.00 | 96.24 |
| 442 | CA | ARG | A | 393 | 51.492 | 74.011 | 9.916 | 1.00 | 100.53 |
| 443 | C | ARG | A | 393 | 51.412 | 74.350 | 11.403 | 1.00 | 102.30 |
| 444 | O | ARG | A | 393 | 52.422 | 74.327 | 12.107 | 1.00 | 101.15 |
| 445 | CB | ARG | A | 393 | 52.579 | 74.853 | 9.245 | 1.00 | 106.99 |
| 446 | CG | ARG | A | 393 | 52.404 | 76.349 | 9.396 | 1.00 | 109.77 |
| 447 | CB | ARG | A | 393 | 53.493 | 77.079 | 8.636 | 1.00 | 115.73 |
| 448 | NE | ARG | A | 393 | 53.515 | 78.505 | 8.935 | 1.00 | 119.73 |
| 449 | CZ | ARG | A | 393 | 54.333 | 79.373 | 8.350 | 1.00 | 121.60 |
| 450 | NH1 | ARG | A | 393 | 55.194 | 78.955 | 7.430 | 1.00 | 121.59 |
| 451 | NH2 | ARG | A | 393 | 54.292 | 80.655 | 8.684 | 1.00 | 121.49 |
| 452 | N | ASN | A | 394 | 50.209 | 74.658 | 11.876 | 1.00 | 103.98 |
| 453 | CA | ASN | A | 394 | 50.005 | 74.970 | 13.281 | 1.00 | 106.60 |
| 454 | C | ASN | A | 394 | 50.275 | 73.682 | 14.051 | 1.00 | 106.89 |
| 455 | O | ASN | A | 394 | 51.084 | 73.650 | 14.978 | 1.00 | 108.55 |
| 456 | CB | ASN | A | 394 | 48.564 | 75.427 | 13.520 | 1.00 | 108.97 |
| 457 | CG | ASN | A | 394 | 48.326 | 75.891 | 14.947 | 1.00 | 112.83 |
| 458 | OD1 | ASN | A | 394 | 47.190 | 76.141 | 15.348 | 1.00 | 114.53 |
| 459 | ND2 | ASN | A | 394 | 49.402 | 76.016 | 15.719 | 1.00 | 114.40 |
| 460 | N | GLY | A | 395 | 49.593 | 72.616 | 13.648 | 1.00 | 105.53 |
| 461 | CA | GLY | A | 395 | 49.771 | 71.331 | 14.298 | 1.00 | 101.15 |
| 462 | C | GLY | A | 395 | 48.585 | 70.423 | 14.050 | 1.00 | 97.82 |
| 463 | O | GLY | A | 395 | 48.125 | 69.721 | 14.952 | 1.00 | 99.41 |
| 464 | N | THR | A | 396 | 48.084 | 70.423 | 12.822 | 1.00 | 94.50 |
| 465 | CA | THR | A | 396 | 46.937 | 69.589 | 12.522 | 1.00 | 89.02 |
| 466 | C | THR | A | 396 | 46.763 | 69.174 | 11.071 | 1.00 | 83.93 |
| 467 | O | THR | A | 396 | 46.477 | 70.000 | 10.204 | 1.00 | 83.27 |
| 468 | CB | THR | A | 396 | 45.625 | 70.278 | 12.970 | 1.00 | 92.03 |
| 469 | OG1 | THR | A | 396 | 45.648 | 70.484 | 14.388 | 1.00 | 94.17 |
| 470 | CG2 | THR | A | 396 | 44.414 | 69.421 | 12.596 | 1.00 | 95.48 |
| 471 | N | LEU | A | 397 | 46.942 | 67.882 | 10.821 | 1.00 | 79.69 |
| 472 | CA | LEU | A | 397 | 46.729 | 67.320 | 9.500 | 1.00 | 74.75 |
| 473 | C | LEU | A | 397 | 45.295 | 66.827 | 9.604 | 1.00 | 71.50 |
| 474 | O | LEU | A | 397 | 45.052 | 65.720 | 10.081 | 1.00 | 74.81 |
| 475 | CB | LEU | A | 397 | 47.640 | 66.120 | 9.240 | 1.00 | 75.72 |
| 476 | CG | LEU | A | 397 | 47.188 | 65.250 | 8.053 | 1.00 | 70.67 |
| 477 | CB1 | LEU | A | 397 | 47.668 | 65.858 | 6.738 | 1.00 | 69.05 |
| 478 | CB2 | LEU | A | 397 | 47.723 | 63.832 | 8.213 | 1.00 | 72.96 |
| 479 | N | THR | A | 398 | 44.348 | 67.659 | 9.195 | 1.00 | 67.61 |
| 480 | CA | THR | A | 398 | 42.945 | 67.286 | 9.252 | 1.00 | 61.30 |
| 481 | C | THR | A | 398 | 42.559 | 66.393 | 8.076 | 1.00 | 56.61 |
| 482 | O | THR | A | 398 | 42.960 | 66.626 | 6.934 | 1.00 | 54.95 |
| 483 | CB | THR | A | 398 | 42.044 | 68.530 | 9.248 | 1.00 | 61.75 |
| 484 | OG1 | THR | A | 398 | 42.225 | 69.255 | 10.471 | 1.00 | 65.40 |
| 485 | CG2 | THR | A | 398 | 40.584 | 68.129 | 9.108 | 1.00 | 63.39 |
| 486 | N | VAL | A | 399 | 41.783 | 65.359 | 8.371 | 1.00 | 51.84 |
| 487 | CA | VAL | A | 399 | 41.321 | 64.437 | 7.349 | 1.00 | 48.05 |
| 488 | C | VAL | A | 399 | 39.870 | 64.085 | 7.610 | 1.00 | 44.81 |
| 489 | O | VAL | A | 399 | 39.502 | 63.689 | 8.719 | 1.00 | 44.41 |
| 490 | CB | VAL | A | 399 | 42.132 | 63.134 | 7.349 | 1.00 | 49.35 |
| 491 | CG1 | VAL | A | 399 | 41.590 | 62.191 | 6.295 | 1.00 | 49.43 |
| 492 | CG2 | VAL | A | 399 | 43.594 | 63.432 | 7.090 | 1.00 | 53.10 |
| 493 | N | THR | A | 400 | 39.036 | 64.246 | 6.596 | 1.00 | 42.44 |
| 494 | CA | THR | A | 400 | 37.635 | 63.909 | 6.757 | 1.00 | 40.44 |
| 495 | C | THR | A | 400 | 37.151 | 63.068 | 5.602 | 1.00 | 38.07 |
| 496 | O | THR | A | 400 | 37.692 | 63.117 | 4.498 | 1.00 | 36.13 |
| 497 | CB | THR | A | 400 | 36.731 | 65.152 | 6.834 | 1.00 | 39.64 |
| 498 | OG1 | THR | A | 400 | 36.638 | 65.755 | 5.539 | 1.00 | 43.72 |
| 499 | CG2 | THR | A | 400 | 37.278 | 66.158 | 7.822 | 1.00 | 38.32 |
| 500 | N | SER | A | 401 | 36.140 | 62.265 | 5.884 | 1.00 | 36.90 |
| 501 | CA | SER | A | 401 | 35.531 | 61.432 | 4.876 | 1.00 | 35.46 |
| 502 | C | SER | A | 401 | 34.043 | 61.612 | 5.084 | 1.00 | 33.95 |
| 503 | O | SER | A | 401 | 33.538 | 61.447 | 6.192 | 1.00 | 34.80 |
| 504 | CB | SER | A | 401 | 35.906 | 59.962 | 5.060 | 1.00 | 36.91 |
| 505 | OG | SER | A | 401 | 35.299 | 59.183 | 4.040 | 1.00 | 40.84 |
| 506 | N | THR | A | 402 | 33.352 | 61.965 | 4.014 | 1.00 | 32.28 |
| 507 | CA | THR | A | 402 | 31.928 | 62.178 | 4.068 | 1.00 | 31.85 |
| 508 | C | THR | A | 402 | 31.245 | 61.092 | 3.264 | 1.00 | 31.76 |
| 509 | O | THR | A | 402 | 31.554 | 60.863 | 2.090 | 1.00 | 30.55 |
| 510 | CB | THR | A | 402 | 31.570 | 63.549 | 3.505 | 1.00 | 31.33 |
| 511 | OG1 | THR | A | 402 | 32.284 | 64.542 | 4.238 | 1.00 | 32.83 |
| 512 | CG2 | THR | A | 402 | 30.078 | 63.818 | 3.632 | 1.00 | 32.70 |
| 513 | N | LEU | A | 403 | 30.299 | 60.433 | 3.912 | 1.00 | 30.72 |
| 514 | CA | LEU | A | 403 | 29.582 | 59.343 | 3.300 | 1.00 | 30.33 |
| 515 | C | LEU | A | 403 | 28.115 | 59.673 | 3.097 | 1.00 | 29.66 |
| 516 | O | LEU | A | 403 | 27.415 | 59.992 | 4.052 | 1.00 | 28.06 |
| 517 | CB | LEU | A | 403 | 29.698 | 58.098 | 4.187 | 1.00 | 28.34 |
| 518 | CG | LEU | A | 403 | 28.968 | 56.831 | 3.719 | 1.00 | 32.10 |
| 519 | CB1 | LEU | A | 403 | 29.777 | 56.175 | 2.597 | 1.00 | 31.94 |
| 520 | CD2 | LEU | A | 403 | 28.810 | 55.844 | 4.876 | 1.00 | 30.42 |
| 521 | N | PRO | A | 404 | 27.638 | 59.617 | 1.841 | 1.00 | 31.48 |
| 522 | CA | PRO | A | 404 | 26.233 | 59.896 | 1.534 | 1.00 | 33.20 |
| 523 | C | PRO | A | 404 | 25.451 | 58.759 | 2.194 | 1.00 | 34.02 |
| 524 | O | PRO | A | 404 | 25.845 | 57.601 | 2.104 | 1.00 | 32.25 |
| 525 | CB | PRO | A | 404 | 26.183 | 59.812 | 0.010 | 1.00 | 33.28 |
| 526 | CG | PRO | A | 404 | 27.590 | 60.165 | 0.400 | 1.00 | 31.82 |
| 527 | CD | PRO | A | 404 | 28.413 | 59.414 | 0.603 | 1.00 | 31.26 |
| 528 | N | VAL | A | 405 | 24.362 | 59.097 | 2.865 | 1.00 | 34.94 |
| 529 | CA | VAL | A | 405 | 23.556 | 58.117 | 3.564 | 1.00 | 38.63 |
| 530 | C | VAL | A | 405 | 22.128 | 58.059 | 3.032 | 1.00 | 40.02 |
| 531 | O | VAL | A | 405 | 21.573 | 59.063 | 2.598 | 1.00 | 40.31 |
| 532 | CB | VAL | A | 405 | 23.548 | 58.457 | 5.082 | 1.00 | 42.09 |
| 533 | CG1 | VAL | A | 405 | 22.128 | 58.490 | 5.632 | 1.00 | 43.95 |
| 534 | CG2 | VAL | A | 405 | 24.405 | 57.456 | 5.827 | 1.00 | 41.76 |
| 535 | N | GLY | A | 406 | 21.536 | 56.872 | 3.061 | 1.00 | 42.35 |
| 536 | CA | GLY | A | 406 | 20.168 | 56.741 | 2.597 | 1.00 | 43.35 |
| 537 | C | GLY | A | 406 | 19.246 | 57.422 | 3.587 | 1.00 | 43.87 |
| 538 | O | GLY | A | 406 | 19.446 | 57.314 | 4.797 | 1.00 | 42.68 |
| 539 | N | THR | A | 407 | 18.248 | 58.134 | 3.077 | 1.00 | 44.40 |
| 540 | CA | THR | A | 407 | 17.294 | 58.840 | 3.923 | 1.00 | 47.37 |
| 541 | C | THR | A | 407 | 16.473 | 57.887 | 4.778 | 1.00 | 48.27 |
| 542 | O | THR | A | 407 | 16.392 | 58.051 | 5.993 | 1.00 | 48.77 |
| 543 | CB | THR | A | 407 | 16.312 | 59.678 | 3.078 | 1.00 | 48.44 |
| 544 | OG1 | THR | A | 407 | 17.001 | 60.791 | 2.504 | 1.00 | 50.00 |
| 545 | CG2 | THR | A | 407 | 15.164 | 60.187 | 3.938 | 1.00 | 53.23 |
| 546 | N | ARG | A | 408 | 15.854 | 56.903 | 4.130 | 1.00 | 48.10 |
| 547 | CA | ARG | A | 408 | 15.021 | 55.935 | 4.821 | 1.00 | 48.54 |
| 548 | C | ARG | A | 408 | 15.827 | 55.060 | 5.765 | 1.00 | 48.48 |
| 549 | O | ARG | A | 408 | 15.418 | 54.831 | 6.903 | 1.00 | 47.34 |
| 550 | CB | ARG | A | 408 | 14.275 | 55.070 | 3.810 | 1.00 | 50.16 |
| 551 | N | ASP | A | 409 | 16.966 | 54.567 | 5.294 | 1.00 | 48.72 |
| 552 | CA | ASP | A | 409 | 17.813 | 53.714 | 6.121 | 1.00 | 50.88 |
| 553 | C | ASP | A | 409 | 18.179 | 54.413 | 7.426 | 1.00 | 49.92 |
| 554 | O | ASP | A | 409 | 18.132 | 53.811 | 8.502 | 1.00 | 49.76 |
| 555 | CB | ASP | A | 409 | 19.088 | 53.340 | 5.362 | 1.00 | 56.18 |
| 556 | CG | ASP | A | 409 | 18.799 | 52.587 | 4.074 | 1.00 | 64.80 |
| 557 | OD1 | ASP | A | 409 | 18.118 | 51.538 | 4.144 | 1.00 | 71.41 |
| 558 | OD2 | ASP | A | 409 | 19.247 | 53.039 | 2.995 | 1.00 | 68.29 |
| 559 | N | TRP | A | 410 | 18.533 | 55.692 | 7.327 | 1.00 | 48.36 |
| 560 | CA | TRP | A | 410 | 18.914 | 56.458 | 8.503 | 1.00 | 46.32 |
| 561 | C | TRP | A | 410 | 17.736 | 56.647 | 9.447 | 1.00 | 46.21 |
| 562 | O | TRP | A | 410 | 17.869 | 56.489 | 10.660 | 1.00 | 44.64 |
| 563 | CB | TRP | A | 410 | 19.480 | 57.826 | 8.108 | 1.00 | 42.82 |
| 564 | CG | TRP | A | 410 | 19.890 | 58.599 | 9.304 | 1.00 | 39.31 |
| 565 | CD1 | TRP | A | 410 | 19.133 | 59.496 | 9.997 | 1.00 | 37.29 |
| 566 | CD2 | TRP | A | 410 | 21.093 | 58.419 | 10.057 | 1.00 | 35.49 |
| 567 | NE1 | TRP | A | 410 | 19.781 | 59.874 | 11.143 | 1.00 | 39.24 |
| 568 | CE2 | TRP | A | 410 | 20.987 | 59.228 | 11.205 | 1.00 | 32.76 |
| 569 | CE3 | TRP | A | 410 | 22.249 | 57.644 | 9.872 | 1.00 | 32.07 |
| 570 | CZ2 | TRP | A | 410 | 21.994 | 59.291 | 12.176 | 1.00 | 29.59 |
| 571 | CZ3 | TRP | A | 410 | 23.248 | 57.704 | 10.831 | 1.00 | 33.68 |
| 572 | CR2 | TRP | A | 410 | 23.111 | 58.527 | 11.975 | 1.00 | 30.56 |
| 573 | N | ILE | A | 411 | 16.584 | 56.989 | 8.888 | 1.00 | 48.11 |
| 574 | CA | ILE | A | 411 | 15.391 | 57.192 | 9.699 | 1.00 | 50.90 |
| 575 | C | ILE | A | 411 | 14.951 | 55.888 | 10.358 | 1.00 | 52.46 |
| 576 | O | ILE | A | 411 | 14.371 | 55.902 | 11.442 | 1.00 | 53.34 |
| 577 | CB | ILE | A | 411 | 14.234 | 57.755 | 8.854 | 1.00 | 51.58 |
| 578 | CG1 | ILE | A | 411 | 14.613 | 59.144 | 8.330 | 1.00 | 52.81 |
| 579 | CG2 | ILE | A | 411 | 12.966 | 57.828 | 9.685 | 1.00 | 51.95 |
| 580 | CD1 | ILE | A | 411 | 13.566 | 59.777 | 7.431 | 1.00 | 59.38 |
| 581 | N | GLU | A | 412 | 15.252 | 54.763 | 9.715 | 1.00 | 53.21 |
| 582 | CA | GLU | A | 412 | 14.872 | 53.463 | 10.248 | 1.00 | 53.12 |
| 583 | C | GLU | A | 412 | 15.853 | 52.919 | 11.269 | 1.00 | 51.24 |
| 584 | O | GLU | A | 412 | 15.610 | 51.864 | 11.848 | 1.00 | 51.40 |
| 585 | CB | GLU | A | 412 | 14.691 | 52.448 | 9.119 | 1.00 | 59.71 |
| 586 | CG | GLU | A | 412 | 13.362 | 52.578 | 8.383 | 1.00 | 70.52 |
| 587 | CD | GLU | A | 412 | 13.201 | 51.540 | 7.290 | 1.00 | 78.58 |
| 588 | OE1 | GLU | A | 412 | 13.402 | 50.338 | 7.573 | 1.00 | 83.44 |
| 589 | OE2 | GLU | A | 412 | 12.868 | 51.924 | 6.150 | 1.00 | 82.10 |
| 590 | N | GLY | A | 413 | 16.966 | 53.618 | 11.477 | 1.00 | 48.47 |
| 591 | CA | GLY | A | 413 | 17.923 | 53.173 | 12.474 | 1.00 | 44.62 |
| 592 | C | GLY | A | 413 | 19.213 | 52.484 | 12.068 | 1.00 | 44.15 |
| 593 | O | GLY | A | 413 | 19.882 | 51.913 | 12.927 | 1.00 | 43.50 |
| 594 | N | GLU | A | 414 | 19.588 | 52.515 | 10.793 | 1.00 | 42.78 |
| 595 | CA | GLU | A | 414 | 20.843 | 51.878 | 10.406 | 1.00 | 40.78 |
| 596 | C | GLU | A | 414 | 21.948 | 52.495 | 11.249 | 1.00 | 39.97 |
| 597 | O | GLU | A | 414 | 21.842 | 53.645 | 11.690 | 1.00 | 37.43 |
| 598 | CB | CLD | A | 414 | 21.155 | 52.111 | 8.918 | 1.00 | 41.96 |
| 599 | CG | GLU | A | 414 | 22.519 | 51.566 | 8.441 | 1.00 | 44.09 |
| 600 | CD | GLU | A | 414 | 22.582 | 50.036 | 8.360 | 1.00 | 49.01 |
| 601 | OE1 | GLU | A | 414 | 23.051 | 49.387 | 9.325 | 1.00 | 46.84 |
| 602 | OE2 | GLU | A | 414 | 22.152 | 49.479 | 7.327 | 1.00 | 49.89 |
| 603 | N | THR | A | 415 | 23.001 | 51.721 | 11.485 | 1.00 | 39.56 |
| 604 | CA | THR | A | 415 | 24.136 | 52.197 | 12.253 | 1.00 | 40.76 |
| 605 | C | THR | A | 415 | 25.369 | 52.113 | 11.362 | 1.00 | 40.07 |
| 606 | O | THR | A | 415 | 25.640 | 51.067 | 10.777 | 1.00 | 42.10 |
| 607 | CB | THR | A | 415 | 24.327 | 51.362 | 13.544 | 1.00 | 44.69 |
| 608 | OG1 | THR | A | 415 | 25.707 | 51.378 | 13.929 | 1.00 | 51.98 |
| 609 | CG2 | THR | A | 415 | 23.856 | 49.948 | 13.342 | 1.00 | 50.00 |
| 610 | N | TYR | A | 416 | 26.100 | 53.220 | 11.240 | 1.00 | 37.57 |
| 611 | CA | TYR | A | 416 | 27.282 | 53.273 | 10.386 | 1.00 | 34.50 |
| 612 | C | TYR | A | 416 | 28.549 | 53.335 | 11.201 | 1.00 | 36.77 |
| 613 | O | TYR | A | 416 | 28.562 | 53.894 | 12.300 | 1.00 | 35.94 |
| 614 | CB | TYR | A | 416 | 27.221 | 54.484 | 9.466 | 1.00 | 34.15 |
| 615 | CG | TYR | A | 416 | 26.003 | 54.515 | 8.586 | 1.00 | 30.65 |
| 616 | CD1 | TYR | A | 416 | 24.744 | 54.823 | 9.108 | 1.00 | 32.53 |
| 617 | CD2 | TYR | A | 416 | 26.101 | 54.211 | 7.235 | 1.00 | 31.72 |
| 618 | CE1 | TYR | A | 416 | 23.616 | 54.828 | 8.297 | 1.00 | 34.27 |
| 619 | CE2 | TYR | A | 416 | 24.981 | 54.210 | 6.420 | 1.00 | 32.11 |
| 620 | CZ | TYR | A | 416 | 23.744 | 54.520 | 6.956 | 1.00 | 33.86 |
| 621 | OH | TYR | A | 416 | 22.636 | 54.518 | 6.139 | 1.00 | 41.22 |
| 622 | N | GLN | A | 417 | 29.624 | 52.771 | 10.659 | 1.00 | 37.58 |
| 623 | CA | GLN | A | 417 | 30.878 | 52.752 | 11.386 | 1.00 | 39.43 |
| 624 | C | GLN | A | 417 | 32.062 | 53.259 | 10.582 | 1.00 | 39.40 |
| 625 | O | GLN | A | 417 | 32.227 | 52.936 | 9.406 | 1.00 | 39.63 |
| 626 | CB | GLN | A | 417 | 31.179 | 51.331 | 11.889 | 1.00 | 41.37 |
| 627 | CG | GLN | A | 417 | 32.386 | 51.250 | 12.822 | 1.00 | 51.81 |
| 628 | CD | GLN | A | 417 | 32.744 | 49.827 | 13.214 | 1.00 | 61.69 |
| 629 | OE1 | CLN | A | 417 | 33.229 | 49.045 | 12.390 | 1.00 | 67.15 |
| 630 | NE2 | CLN | A | 417 | 32.504 | 49.481 | 14.479 | 1.00 | 63.64 |
| 631 | N | CYS | A | 418 | 32.888 | 54.057 | 11.236 | 1.00 | 39.29 |
| 632 | CA | CYS | A | 418 | 34.083 | 54.581 | 10.611 | 1.00 | 41.80 |
| 633 | C | CYS | A | 418 | 35.250 | 53.880 | 11.282 | 1.00 | 42.50 |
| 634 | O | CYS | A | 418 | 35.409 | 53.954 | 12.500 | 1.00 | 43.33 |
| 635 | CB | CYS | A | 418 | 34.208 | 56.099 | 10.825 | 1.00 | 41.95 |
| 636 | SG | CYS | A | 418 | 35.696 | 56.805 | 10.043 | 1.00 | 54.15 |
| 637 | N | ARG | A | 419 | 36.049 | 53.182 | 10.491 | 1.00 | 43.98 |
| 638 | CA | ARG | A | 419 | 37.225 | 52.497 | 11.007 | 1.00 | 47.51 |
| 639 | C | ARG | A | 419 | 38.428 | 53.323 | 10.558 | 1.00 | 48.11 |
| 640 | O | ARG | A | 419 | 38.741 | 53.381 | 9.370 | 1.00 | 47.85 |
| 641 | CB | ARG | A | 419 | 37.289 | 51.068 | 10.452 | 1.00 | 49.08 |
| 642 | CG | ARG | A | 419 | 38.642 | 50.397 | 10.586 | 1.00 | 54.11 |
| 643 | CD | ARG | A | 419 | 38.554 | 48.892 | 10.304 | 1.00 | 64.71 |
| 644 | NE | ARG | A | 419 | 39.868 | 48.275 | 10.137 | 1.00 | 64.09 |
| 645 | CZ | ARG | A | 419 | 40.493 | 48.163 | 8.968 | 1.00 | 70.31 |
| 646 | NH1 | ARG | A | 419 | 39.919 | 48.619 | 7.861 | 1.00 | 68.32 |
| 647 | NH2 | ARG | A | 419 | 41.699 | 47.608 | 8.905 | 1.00 | 71.53 |
| 648 | N | VAL | A | 420 | 39.076 | 53.990 | 11.508 | 1.00 | 51.04 |
| 649 | CA | VAL | A | 420 | 40.231 | 54.824 | 11.200 | 1.00 | 56.22 |
| 650 | C | VAL | A | 420 | 41.532 | 54.039 | 11.354 | 1.00 | 61.25 |
| 651 | O | VAL | A | 420 | 41.780 | 53.422 | 12.393 | 1.00 | 60.11 |
| 652 | CB | VAL | A | 420 | 40.295 | 56.064 | 12.115 | 1.00 | 54.44 |
| 653 | CG1 | VAL | A | 420 | 41.494 | 56.922 | 11.729 | 1.00 | 52.69 |
| 654 | CG2 | VAL | A | 420 | 39.003 | 56.868 | 12.005 | 1.00 | 55.45 |
| 655 | N | THR | A | 421 | 42.359 | 54.071 | 10.314 | 1.00 | 66.23 |
| 656 | CA | THR | A | 421 | 43.631 | 53.356 | 10.326 | 1.00 | 73.18 |
| 657 | C | THR | A | 421 | 44.834 | 54.266 | 10.130 | 1.00 | 76.87 |
| 658 | O | THR | A | 421 | 45.157 | 54.632 | 9.000 | 1.00 | 77.93 |
| 659 | CB | THE | A | 421 | 43.671 | 52.283 | 9.225 | 1.00 | 73.75 |
| 660 | OG1 | THE | A | 421 | 42.628 | 51.328 | 9.451 | 1.00 | 77.71 |
| 661 | CG2 | THE | A | 421 | 45.009 | 51.567 | 9.227 | 1.00 | 75.35 |
| 662 | N | HIS | A | 422 | 45.499 | 54.624 | 11.226 | 1.00 | 81.02 |
| 663 | CA | HIS | A | 422 | 46.678 | 55.483 | 11.151 | 1.00 | 86.07 |
| 664 | C | HIS | A | 422 | 47.914 | 54.596 | 11.278 | 1.00 | 89.67 |
| 665 | O | HIS | A | 422 | 47.947 | 53.680 | 12.098 | 1.00 | 90.00 |
| 666 | CB | HIS | A | 422 | 46.660 | 56.529 | 12.269 | 1.00 | 87.43 |
| 667 | CG | HIS | A | 422 | 47.548 | 57.708 | 12.008 | 1.00 | 90.73 |
| 668 | ND1 | HIS | A | 422 | 47.397 | 58.524 | 10.909 | 1.00 | 93.83 |
| 669 | CD2 | HIS | A | 422 | 48.600 | 58.204 | 12.704 | 1.00 | 91.89 |
| 670 | CE1 | HIS | A | 422 | 48.318 | 59.473 | 10.936 | 1.00 | 94.70 |
| 671 | NE2 | HIS | A | 422 | 49.060 | 59.300 | 12.015 | 1.00 | 93.25 |
| 672 | N | PRO | A | 423 | 48.954 | 54.871 | 10.474 | 1.00 | 92.64 |
| 673 | CA | PRO | A | 423 | 50.209 | 54.111 | 10.460 | 1.00 | 95.19 |
| 674 | C | PRO | A | 423 | 50.838 | 53.628 | 11.775 | 1.00 | 97.06 |
| 675 | O | PRO | A | 423 | 51.064 | 52.431 | 11.945 | 1.00 | 97.57 |
| 676 | CB | PRO | A | 423 | 51.155 | 55.024 | 9.667 | 1.00 | 95.56 |
| 677 | CG | PRO | A | 423 | 50.566 | 56.391 | 9.853 | 1.00 | 93.94 |
| 678 | CD | PRO | A | 423 | 49.105 | 56.112 | 9.696 | 1.00 | 93.12 |
| 679 | N | HIS | A | 424 | 51.105 | 54.542 | 12.701 | 1.00 | 98.71 |
| 680 | CA | HIS | A | 424 | 51.769 | 54.190 | 13.957 | 1.00 | 100.16 |
| 681 | C | HIS | A | 424 | 51.021 | 53.496 | 15.095 | 1.00 | 99.67 |
| 682 | O | HIS | A | 424 | 51.609 | 52.690 | 15.816 | 1.00 | 99.61 |
| 683 | CB | HIS | A | 424 | 52.451 | 55.435 | 14.522 | 1.00 | 102.42 |
| 684 | CG | HIS | A | 424 | 53.475 | 56.022 | 13.605 | 1.00 | 105.47 |
| 685 | ND1 | HIS | A | 424 | 53.372 | 57.298 | 13.092 | 1.00 | 107.10 |
| 686 | CG2 | HIS | A | 424 | 54.639 | 55.521 | 13.133 | 1.00 | 97.46 |
| 687 | CE1 | HIS | A | 424 | 54.430 | 57.556 | 12.345 | 1.00 | 106.81 |
| 688 | NE2 | HIS | A | 424 | 55.215 | 56.494 | 12.353 | 1.00 | 106.61 |
| 689 | N | LEU | A | 425 | 49.742 | 53.798 | 15.267 | 1.00 | 99.19 |
| 690 | CA | LEU | A | 425 | 48.972 | 53.216 | 16.363 | 1.00 | 99.01 |
| 691 | C | LEU | A | 425 | 48.692 | 51.714 | 16.284 | 1.00 | 98.36 |
| 692 | O | LEU | A | 425 | 48.479 | 51.160 | 15.204 | 1.00 | 98.39 |
| 693 | CB | LEU | A | 425 | 47.671 | 53.994 | 16.507 | 1.00 | 100.66 |
| 694 | CG | LEU | A | 425 | 47.969 | 55.495 | 16.467 | 1.00 | 101.95 |
| 695 | CD1 | LEU | A | 425 | 46.683 | 56.285 | 16.569 | 1.00 | 102.59 |
| 696 | CD2 | LEU | A | 425 | 48.923 | 55.856 | 17.598 | 1.00 | 103.85 |
| 697 | N | PRO | A | 426 | 48.685 | 51.038 | 17.448 | 1.00 | 97.57 |
| 698 | CA | PRO | A | 426 | 48.435 | 49.596 | 17.540 | 1.00 | 96.82 |
| 699 | C | PRO | A | 426 | 47.075 | 49.186 | 16.992 | 1.00 | 95.53 |
| 700 | O | PRO | A | 426 | 46.990 | 48.560 | 15.937 | 1.00 | 95.77 |
| 701 | CB | PRO | A | 426 | 48.570 | 49.318 | 19.037 | 1.00 | 97.13 |
| 702 | CG | PRO | A | 426 | 48.106 | 50.605 | 19.660 | 1.00 | 97.88 |
| 703 | CD | PRO | A | 426 | 48.802 | 51.631 | 18.793 | 1.00 | 97.70 |
| 704 | N | ARG | A | 427 | 46.015 | 49.536 | 17.713 | 1.00 | 94.16 |
| 705 | CA | ARG | A | 427 | 44.669 | 49.198 | 17.274 | 1.00 | 92.52 |
| 706 | C | ARG | A | 427 | 44.091 | 50.315 | 16.408 | 1.00 | 89.85 |
| 707 | O | ARG | A | 427 | 44.487 | 51.477 | 16.517 | 1.00 | 89.26 |
| 708 | CB | ARG | A | 427 | 43.749 | 48.948 | 18.478 | 1.00 | 97.34 |
| 709 | CG | ARG | A | 427 | 42.381 | 48.398 | 18.077 | 1.00 | 99.32 |
| 710 | CD | ARG | A | 427 | 41.428 | 48.193 | 19.253 | 1.00 | 105.99 |
| 711 | NE | ARG | A | 427 | 40.197 | 47.532 | 18.815 | 1.00 | 111.10 |
| 712 | CZ | ARG | A | 427 | 39.140 | 47.298 | 19.588 | 1.00 | 111.66 |
| 713 | NH1 | ARG | A | 427 | 39.143 | 47.673 | 20.861 | 1.00 | 111.70 |
| 714 | NH2 | ARG | A | 427 | 38.078 | 46.679 | 19.087 | 1.00 | 110.09 |
| 715 | N | ALA | A | 428 | 43.158 | 49.949 | 15.538 | 1.00 | 86.56 |
| 716 | CA | ALA | A | 428 | 42.513 | 50.910 | 14.661 | 1.00 | 82.70 |
| 717 | C | ALA | A | 428 | 41.389 | 51.602 | 15.423 | 1.00 | 80.03 |
| 718 | O | ALA | A | 428 | 40.711 | 50.981 | 16.239 | 1.00 | 80.29 |
| 719 | CB | ALA | A | 428 | 41.953 | 50.200 | 13.437 | 1.00 | 83.85 |
| 720 | N | LEU | A | 429 | 41.203 | 52.892 | 15.164 | 1.00 | 76.76 |
| 721 | CA | LEU | A | 429 | 40.144 | 53.651 | 15.815 | 1.00 | 72.72 |
| 722 | C | LEU | A | 429 | 38.820 | 53.289 | 15.164 | 1.00 | 69.67 |
| 723 | O | LEU | A | 429 | 38.742 | 53.128 | 13.947 | 1.00 | 68.64 |
| 724 | CB | LEU | A | 429 | 40.392 | 55.156 | 15.671 | 1.00 | 73.60 |
| 725 | CG | LEU | A | 429 | 41.169 | 55.847 | 16.793 | 1.00 | 73.35 |
| 726 | CD1 | LEU | A | 429 | 41.498 | 57.280 | 16.404 | 1.00 | 72.09 |
| 727 | CD2 | LEU | A | 429 | 40.333 | 55.813 | 18.065 | 1.00 | 76.40 |
| 728 | N | MET | A | 430 | 37.779 | 53.153 | 15.976 | 1.00 | 66.83 |
| 729 | CA | MET | A | 430 | 36.468 | 52.814 | 15.449 | 1.00 | 64.81 |
| 730 | C | MET | A | 430 | 35.384 | 53.678 | 16.074 | 1.00 | 61.41 |
| 731 | O | MET | A | 430 | 35.274 | 53.776 | 17.299 | 1.00 | 60.84 |
| 732 | CB | MET | A | 430 | 36.173 | 51.335 | 15.691 | 1.00 | 68.22 |
| 733 | CG | MET | A | 430 | 37.247 | 50.416 | 15.133 | 1.00 | 74.39 |
| 734 | SD | MET | A | 430 | 36.886 | 48.679 | 15.387 | 1.00 | 83.18 |
| 735 | CE | MET | A | 430 | 36.836 | 48.607 | 17.194 | 1.00 | 84.12 |
| 736 | N | ARG | A | 431 | 34.590 | 54.312 | 15.218 | 1.00 | 57.17 |
| 737 | CA | ARG | A | 431 | 33.506 | 55.172 | 15.674 | 1.00 | 54.04 |
| 738 | C | ARG | A | 431 | 32.234 | 54.752 | 14.963 | 1.00 | 50.58 |
| 739 | O | ARG | A | 431 | 32.270 | 54.367 | 13.796 | 1.00 | 50.46 |
| 740 | CB | ARG | A | 431 | 33.825 | 56.641 | 15.364 | 1.00 | 55.16 |
| 741 | CG | ARG | A | 431 | 35.036 | 57.187 | 16.109 | 1.00 | 59.24 |
| 742 | CD | ARG | A | 431 | 34.802 | 57.172 | 17.611 | 1.00 | 63.93 |
| 743 | NE | ARG | A | 431 | 35.983 | 57.586 | 18.363 | 1.00 | 70.30 |
| 744 | CZ | ARG | A | 431 | 36.070 | 57.557 | 19.689 | 1.00 | 76.24 |
| 745 | NH1 | ARG | A | 431 | 35.041 | 57.131 | 20.417 | 1.00 | 77.83 |
| 746 | NH2 | ARG | A | 431 | 37.185 | 57.952 | 20.289 | 1.00 | 77.12 |
| 747 | N | SER | A | 432 | 31.112 | 54.812 | 15.669 | 1.00 | 46.92 |
| 748 | CA | SER | A | 432 | 29.835 | 54.437 | 15.084 | 1.00 | 45.15 |
| 749 | C | SER | A | 432 | 28.784 | 55.482 | 15.398 | 1.00 | 42.49 |
| 750 | O | SER | A | 432 | 28.901 | 56.218 | 16.379 | 1.00 | 42.76 |
| 751 | CB | SER | A | 432 | 29.394 | 53.068 | 15.610 | 1.00 | 44.31 |
| 752 | OG | SER | A | 432 | 29.316 | 53.084 | 17.018 | 1.00 | 49.96 |
| 753 | N | THR | A | 433 | 27.749 | 55.540 | 14.570 | 1.00 | 40.28 |
| 754 | CA | THR | A | 433 | 26.691 | 56.522 | 14.763 | 1.00 | 39.71 |
| 755 | C | THR | A | 433 | 25.356 | 55.996 | 14.257 | 1.00 | 39.49 |
| 756 | O | THR | A | 433 | 25.315 | 55.186 | 13.334 | 1.00 | 40.22 |
| 757 | CB | THR | A | 433 | 27.023 | 57.839 | 14.022 | 1.00 | 38.07 |
| 758 | OG1 | THR | A | 433 | 26.006 | 58.807 | 14.295 | 1.00 | 39.56 |
| 759 | CG2 | THR | A | 433 | 27.088 | 57.609 | 12.518 | 1.00 | 36.99 |
| 760 | N | THR | A | 434 | 24.272 | 56.461 | 14.874 | 1.00 | 39.18 |
| 761 | CA | THR | A | 434 | 22.913 | 56.068 | 14.512 | 1.00 | 40.70 |
| 762 | C | THR | A | 434 | 21.965 | 57.186 | 14.934 | 1.00 | 40.23 |
| 763 | O | THR | A | 434 | 22.364 | 58.091 | 15.659 | 1.00 | 41.18 |
| 764 | CB | THR | A | 434 | 22.484 | 54.784 | 15.256 | 1.00 | 43.01 |
| 765 | OG1 | THR | A | 434 | 22.362 | 55.052 | 16.661 | 1.00 | 47.85 |
| 766 | CG2 | THR | A | 434 | 23.524 | 53.726 | 15.105 | 1.00 | 48.67 |
| 767 | N | LYS | A | 435 | 20.714 | 57.125 | 14.496 | 1.00 | 40.97 |
| 768 | CA | LYS | A | 435 | 19.767 | 58.150 | 14.889 | 1.00 | 45.73 |
| 769 | C | LYS | A | 435 | 19.601 | 58.021 | 16.398 | 1.00 | 48.93 |
| 770 | O | LYS | A | 435 | 19.710 | 56.930 | 16.942 | 1.00 | 48.49 |
| 771 | CB | LYS | A | 435 | 18.434 | 57.937 | 14.194 | 1.00 | 45.94 |
| 772 | N | THR | A | 436 | 19.365 | 59.133 | 17.082 | 1.00 | 52.01 |
| 773 | CA | THR | A | 436 | 19.171 | 59.080 | 18.526 | 1.00 | 54.88 |
| 774 | C | THR | A | 436 | 17.722 | 58.684 | 18.774 | 1.00 | 55.70 |
| 775 | O | THR | A | 436 | 16.823 | 59.146 | 18.077 | 1.00 | 55.21 |
| 776 | CB | THR | A | 436 | 19.438 | 60.454 | 19.191 | 1.00 | 56.24 |
| 777 | OG1 | THR | A | 436 | 20.821 | 60.802 | 19.038 | 1.00 | 60.56 |
| 778 | CG2 | THR | A | 436 | 19.090 | 60.412 | 20.677 | 1.00 | 57.04 |
| 779 | N | SER | A | 437 | 17.501 | 57.810 | 19.748 | 1.00 | 57.93 |
| 780 | CA | SER | A | 437 | 16.149 | 57.380 | 20.080 | 1.00 | 60.42 |
| 781 | C | SER | A | 437 | 15.683 | 58.239 | 21.249 | 1.00 | 60.49 |
| 782 | O | SER | A | 437 | 16.357 | 59.203 | 21.623 | 1.00 | 60.21 |
| 783 | CB | SER | A | 437 | 16.133 | 55.894 | 20.470 | 1.00 | 61.57 |
| 784 | OG | SER | A | 437 | 16.957 | 55.651 | 21.600 | 1.00 | 65.51 |
| 785 | N | GLY | A | 438 | 14.533 | 57.895 | 21.821 | 1.00 | 60.09 |
| 786 | CA | GLY | A | 438 | 14.022 | 58.666 | 22.939 | 1.00 | 58.42 |
| 787 | C | GLY | A | 438 | 12.937 | 59.630 | 22.511 | 1.00 | 57.80 |
| 788 | O | GLY | A | 438 | 12.648 | 59.747 | 21.322 | 1.00 | 56.51 |
| 789 | N | PRO | A | 439 | 12.309 | 60.333 | 23.467 | 1.00 | 57.81 |
| 790 | CA | PRO | A | 439 | 11.242 | 61.295 | 23.185 | 1.00 | 56.19 |
| 791 | C | PRO | A | 439 | 11.692 | 62.422 | 22.263 | 1.00 | 53.91 |
| 792 | O | PRO | A | 439 | 12.886 | 62.709 | 22.147 | 1.00 | 53.91 |
| 793 | CB | PRO | A | 439 | 10.865 | 61.806 | 24.576 | 1.00 | 57.79 |
| 794 | CG | PRO | A | 439 | 11.147 | 60.633 | 25.448 | 1.00 | 58.60 |
| 795 | CD | PRO | A | 439 | 12.485 | 60.173 | 24.921 | 1.00 | 60.65 |
| 796 | N | ARG | A | 440 | 10.722 | 63.055 | 21.612 | 1.00 | 51.09 |
| 797 | CA | ARG | A | 440 | 10.990 | 64.163 | 20.702 | 1.00 | 48.90 |
| 798 | C | ARG | A | 440 | 10.220 | 65.365 | 21.231 | 1.00 | 45.83 |
| 799 | O | ARG | A | 440 | 9.188 | 65.202 | 21.881 | 1.00 | 45.88 |
| 800 | CB | ARG | A | 440 | 10.501 | 63.836 | 19.289 | 1.00 | 49.95 |
| 801 | CG | ARG | A | 440 | 10.919 | 62.473 | 18.771 | 1.00 | 59.03 |
| 802 | CB | ARG | A | 440 | 12.406 | 62.373 | 18.448 | 1.00 | 65.31 |
| 803 | NE | ARG | A | 440 | 12.814 | 60.973 | 18.342 | 1.00 | 72.31 |
| 804 | CZ | ARG | A | 440 | 13.986 | 60.553 | 17.878 | 1.00 | 74.99 |
| 805 | NH1 | ARG | A | 440 | 14.894 | 61.419 | 17.460 | 1.00 | 77.22 |
| 806 | NE2 | ARG | A | 440 | 14.252 | 59.255 | 17.837 | 1.00 | 78.59 |
| 807 | N | ALA | A | 441 | 10.727 | 66.564 | 20.957 | 1.00 | 42.09 |
| 808 | CA | ALA | A | 441 | 10.079 | 67.800 | 21.387 | 1.00 | 39.26 |
| 809 | C | ALA | A | 441 | 10.476 | 68.894 | 20.413 | 1.00 | 38.22 |
| 810 | O | ALA | A | 441 | 11.649 | 69.034 | 20.076 | 1.00 | 38.95 |
| 811 | CB | ALA | A | 441 | 10.511 | 68.170 | 22.798 | 1.00 | 36.56 |
| 812 | N | ALA | A | 442 | 9.496 | 69.666 | 19.966 | 1.00 | 37.22 |
| 813 | CA | ALA | A | 442 | 9.739 | 70.729 | 19.011 | 1.00 | 37.60 |
| 814 | C | ALA | A | 442 | 10.550 | 71.879 | 19.590 | 1.00 | 38.65 |
| 815 | O | ALA | A | 442 | 10.553 | 72.103 | 20.798 | 1.00 | 40.22 |
| 816 | CB | ALA | A | 442 | 8.415 | 71.248 | 18.481 | 1.00 | 37.17 |
| 817 | N | PRO | A | 443 | 11.260 | 72.619 | 18.722 | 1.00 | 38.04 |
| 818 | CA | PRO | A | 443 | 12.089 | 73.765 | 19.104 | 1.00 | 36.79 |
| 819 | C | PRO | A | 443 | 11.224 | 75.019 | 19.243 | 1.00 | 38.14 |
| 820 | O | PRO | A | 443 | 10.236 | 75.174 | 18.521 | 1.00 | 37.39 |
| 821 | CB | PRO | A | 443 | 13.050 | 73.923 | 17.921 | 1.00 | 36.30 |
| 822 | CG | PRO | A | 443 | 12.946 | 72.632 | 17.159 | 1.00 | 36.35 |
| 823 | CD | PRO | A | 443 | 11.512 | 72.249 | 17.320 | 1.00 | 37.85 |
| 824 | N | GLU | A | 444 | 11.594 | 75.894 | 20.173 | 1.00 | 36.28 |
| 825 | CA | GLU | A | 444 | 10.901 | 77.163 | 20.371 | 1.00 | 35.57 |
| 826 | C | GLU | A | 444 | 11.941 | 78.171 | 19.888 | 1.00 | 33.87 |
| 827 | O | GLU | A | 444 | 13.110 | 78.105 | 20.291 | 1.00 | 33.43 |
| 828 | CB | GLU | A | 444 | 10.580 | 77.383 | 21.851 | 1.00 | 38.68 |
| 829 | N | VAL | A | 445 | 11.520 | 79.097 | 19.033 | 1.00 | 30.18 |
| 830 | CA | VAL | A | 445 | 12.432 | 80.070 | 18.450 | 1.00 | 28.24 |
| 831 | C | VAL | A | 445 | 12.136 | 81.523 | 18.811 | 1.00 | 28.44 |
| 832 | O | VAL | A | 445 | 11.000 | 81.975 | 18.721 | 1.00 | 26.78 |
| 833 | CB | VAL | A | 445 | 12.417 | 79.937 | 16.905 | 1.00 | 28.10 |
| 834 | CG1 | VAL | A | 445 | 13.370 | 80.933 | 16.274 | 1.00 | 25.56 |
| 835 | CG2 | VAL | A | 445 | 12.771 | 78.521 | 16.511 | 1.00 | 26.30 |
| 836 | N | TYR | A | 446 | 13.175 | 82.251 | 19.203 | 1.00 | 27.87 |
| 837 | CA | TYR | A | 446 | 13.019 | 83.651 | 19.551 | 1.00 | 27.91 |
| 838 | C | TYR | A | 446 | 14.193 | 84.460 | 19.024 | 1.00 | 27.06 |
| 839 | O | TYR | A | 446 | 15.326 | 84.307 | 19.480 | 1.00 | 27.96 |
| 840 | CB | TYR | A | 446 | 12.898 | 83.836 | 21.074 | 1.00 | 26.45 |
| 841 | CG | TYR | A | 446 | 12.749 | 85.289 | 21.447 | 1.00 | 26.59 |
| 842 | CD1 | TYR | A | 446 | 11.714 | 86.057 | 20.905 | 1.00 | 28.48 |
| 843 | CD2 | TYR | A | 446 | 13.659 | 85.914 | 22.305 | 1.00 | 29.02 |
| 844 | CE1 | TYR | A | 446 | 11.589 | 87.417 | 21.204 | 1.00 | 33.24 |
| 845 | CE2 | TYR | A | 446 | 13.543 | 87.274 | 22.616 | 1.00 | 31.33 |
| 846 | CZ | TYR | A | 446 | 12.506 | 88.015 | 22.059 | 1.00 | 36.01 |
| 847 | OH | TYR | A | 446 | 12.387 | 89.352 | 22.338 | 1.00 | 36.54 |
| 848 | N | ALA | A | 447 | 13.914 | 85.326 | 18.060 | 1.00 | 28.07 |
| 849 | CA | ALA | A | 447 | 14.949 | 86.150 | 17.439 | 1.00 | 27.67 |
| 850 | C | ALA | A | 447 | 14.815 | 87.579 | 17.932 | 1.00 | 29.19 |
| 851 | O | ALA | A | 447 | 13.702 | 88.036 | 18.173 | 1.00 | 31.10 |
| 852 | CB | ALA | A | 447 | 14.802 | 86.098 | 15.931 | 1.00 | 23.97 |
| 853 | N | PHE | A | 448 | 15.929 | 88.293 | 18.079 | 1.00 | 29.56 |
| 854 | CA | PHE | A | 448 | 15.849 | 89.657 | 18.578 | 1.00 | 32.50 |
| 855 | C | PHE | A | 448 | 17.075 | 90.483 | 18.246 | 1.00 | 33.67 |
| 856 | O | PHE | A | 448 | 18.112 | 89.950 | 17.872 | 1.00 | 35.26 |
| 857 | CB | PHE | A | 448 | 15.659 | 89.630 | 20.097 | 1.00 | 35.29 |
| 858 | CG | PHE | A | 448 | 16.849 | 89.077 | 20.838 | 1.00 | 37.04 |
| 859 | CB1 | PHE | A | 448 | 17.912 | 89.909 | 21.195 | 1.00 | 36.66 |
| 860 | CD2 | PHE | A | 448 | 16.934 | 87.714 | 21.127 | 1.00 | 34.95 |
| 861 | CE1 | PHE | A | 448 | 19.049 | 89.382 | 21.824 | 1.00 | 40.91 |
| 862 | CE2 | PHE | A | 448 | 18.066 | 87.178 | 21.753 | 1.00 | 35.99 |
| 863 | CZ | PHE | A | 448 | 19.123 | 88.014 | 22.103 | 1.00 | 35.15 |
| 864 | N | ALA | A | 449 | 16.952 | 91.796 | 18.396 | 1.00 | 35.81 |
| 865 | CA | ALA | A | 449 | 18.066 | 92.693 | 18.131 | 1.00 | 37.63 |
| 866 | C | ALA | A | 449 | 18.617 | 93.253 | 19.438 | 1.00 | 40.96 |
| 867 | O | ALA | A | 449 | 17.885 | 93.441 | 20.403 | 1.00 | 40.14 |
| 868 | CB | ALA | A | 449 | 17.623 | 93.831 | 17.230 | 1.00 | 33.60 |
| 869 | N | THR | A | 450 | 19.918 | 93.509 | 19.448 | 1.00 | 45.40 |
| 870 | CA | THR | A | 450 | 20.606 | 94.071 | 20.595 | 1.00 | 50.82 |
| 871 | C | THR | A | 450 | 20.515 | 95.597 | 20.498 | 1.00 | 53.78 |
| 872 | O | THR | A | 450 | 20.673 | 96.169 | 19.417 | 1.00 | 54.14 |
| 873 | CB | THR | A | 450 | 22.090 | 93.665 | 20.587 | 1.00 | 51.45 |
| 874 | OG1 | THR | A | 450 | 22.192 | 92.242 | 20.450 | 1.00 | 54.12 |
| 875 | CG2 | THR | A | 450 | 22.769 | 94.093 | 21.877 | 1.00 | 55.58 |
| 876 | N | PRO | A | 451 | 20.260 | 96.275 | 21.626 | 1.00 | 56.74 |
| 877 | CA | PRO | A | 451 | 20.157 | 97.737 | 21.623 | 1.00 | 59.05 |
| 878 | C | PRO | A | 451 | 21.427 | 98.391 | 21.080 | 1.00 | 61.00 |
| 879 | O | PRO | A | 451 | 22.535 | 98.080 | 21.521 | 1.00 | 59.71 |
| 880 | CB | PRO | A | 451 | 19.933 | 98.073 | 23.098 | 1.00 | 60.52 |
| 881 | CG | PRO | A | 451 | 19.262 | 96.851 | 23.640 | 1.00 | 61.14 |
| 882 | CD | PRO | A | 451 | 20.049 | 95.739 | 22.981 | 1.00 | 59.39 |
| 883 | N | GLU | A | 452 | 21.272 | 99.285 | 20.112 | 1.00 | 64.30 |
| 884 | CA | GLU | A | 452 | 22.428 | 99.975 | 19.558 | 1.00 | 69.17 |
| 885 | C | GLU | A | 452 | 22.252 | 101.463 | 19.842 | 1.00 | 72.61 |
| 886 | O | GLU | A | 452 | 21.667 | 102.206 | 19.057 | 1.00 | 73.59 |
| 887 | CB | GLU | A | 452 | 22.560 | 99.712 | 18.054 | 1.00 | 65.66 |
| 888 | CG | GLU | A | 452 | 24.015 | 99.566 | 17.577 | 1.00 | 61.46 |
| 889 | CD | GLU | A | 452 | 24.717 | 98.317 | 18.133 | 1.00 | 60.52 |
| 890 | OE1 | GLU | A | 452 | 24.053 | 97.264 | 18.251 | 1.00 | 63.47 |
| 891 | OE2 | GLU | A | 452 | 25.934 | 98.373 | 18.433 | 1.00 | 53.34 |
| 892 | N | THR | A | 453 | 22.763 | 101.869 | 20.997 | 1.00 | 76.75 |
| 893 | CA | THR | A | 453 | 22.693 | 103.242 | 21.481 | 1.00 | 81.00 |
| 894 | C | THR | A | 453 | 23.886 | 104.027 | 20.913 | 1.00 | 82.30 |
| 895 | O | THR | A | 453 | 25.038 | 103.710 | 21.215 | 1.00 | 82.08 |
| 896 | CB | THR | A | 453 | 22.737 | 103.191 | 23.010 | 1.00 | 85.65 |
| 897 | CG | THR | A | 453 | 22.182 | 104.367 | 23.734 | 1.00 | 89.76 |
| 898 | CD1 | THR | A | 453 | 20.939 | 104.921 | 23.588 | 1.00 | 92.34 |
| 899 | CD2 | THR | A | 453 | 22.819 | 105.083 | 24.795 | 1.00 | 94.15 |
| 900 | NH1 | THR | A | 453 | 20.764 | 105.934 | 24.502 | 1.00 | 94.99 |
| 901 | CE2 | THR | A | 453 | 21.904 | 106.055 | 25.255 | 1.00 | 95.56 |
| 902 | CE3 | THR | A | 453 | 24.079 | 104.995 | 25.407 | 1.00 | 95.36 |
| 903 | CZ2 | THR | A | 453 | 22.208 | 106.935 | 26.300 | 1.00 | 97.14 |
| 904 | CZ3 | THR | A | 453 | 24.380 | 105.869 | 26.445 | 1.00 | 95.61 |
| 905 | CH2 | THR | A | 453 | 23.447 | 106.826 | 26.880 | 1.00 | 97.12 |
| 906 | N | PRO | A | 454 | 23.619 | 105.058 | 20.080 | 1.00 | 84.10 |
| 907 | CA | PRO | A | 454 | 24.606 | 105.931 | 19.422 | 1.00 | 87.24 |
| 908 | C | PRO | A | 454 | 25.892 | 106.316 | 20.162 | 1.00 | 90.52 |
| 909 | O | PRO | A | 454 | 26.088 | 105.978 | 21.333 | 1.00 | 89.75 |
| 910 | CB | PRO | A | 454 | 23.775 | 107.150 | 18.988 | 1.00 | 84.91 |
| 911 | CG | PRO | A | 454 | 22.475 | 107.017 | 19.740 | 1.00 | 83.62 |
| 912 | CD | PRO | A | 454 | 22.259 | 105.537 | 19.793 | 1.00 | 84.60 |
| 913 | N | GLY | A | 455 | 26.751 | 107.055 | 19.464 | 1.00 | 91.72 |
| 914 | CA | GLY | A | 455 | 28.048 | 107.413 | 20.012 | 1.00 | 94.13 |
| 915 | C | GLY | A | 455 | 28.730 | 106.132 | 19.601 | 1.00 | 96.28 |
| 916 | O | GLY | A | 455 | 28.613 | 105.125 | 20.301 | 1.00 | 97.74 |
| 917 | N | SER | A | 456 | 29.442 | 106.142 | 18.478 | 1.00 | 99.11 |
| 918 | CA | SER | A | 456 | 29.974 | 104.869 | 18.023 | 1.00 | 96.15 |
| 919 | C | SER | A | 456 | 31.407 | 104.465 | 17.676 | 1.00 | 91.50 |
| 920 | O | SER | A | 456 | 32.292 | 105.284 | 17.333 | 1.00 | 88.52 |
| 921 | CB | SER | A | 456 | 29.092 | 104.378 | 16.879 | 1.00 | 95.05 |
| 922 | OG | SER | A | 456 | 28.910 | 105.375 | 15.884 | 1.00 | 110.76 |
| 923 | N | ARG | A | 457 | 31.541 | 103.136 | 17.752 | 1.00 | 87.73 |
| 924 | CA | ARG | A | 457 | 32.664 | 102.251 | 17.520 | 1.00 | 87.81 |
| 925 | C | ARG | A | 457 | 31.678 | 101.103 | 17.647 | 1.00 | 86.30 |
| 926 | O | ARG | A | 457 | 31.026 | 100.998 | 18.697 | 1.00 | 90.57 |
| 927 | CB | ARG | A | 457 | 33.634 | 102.261 | 18.675 | 1.00 | 94.67 |
| 928 | N | ASP | A | 458 | 31.512 | 100.259 | 16.627 | 1.00 | 87.32 |
| 929 | CA | ASP | A | 458 | 30.441 | 99.286 | 16.780 | 1.00 | 80.43 |
| 930 | C | ASP | A | 458 | 30.422 | 97.963 | 16.080 | 1.00 | 72.83 |
| 931 | O | ASP | A | 458 | 31.410 | 97.437 | 15.599 | 1.00 | 73.96 |
| 932 | CB | ASP | A | 458 | 29.150 | 99.985 | 16.385 | 1.00 | 83.19 |
| 933 | CG | ASP | A | 458 | 29.102 | 100.300 | 14.890 | 1.00 | 85.09 |
| 934 | OD1 | ASP | A | 458 | 30.065 | 100.906 | 14.378 | 1.00 | 91.25 |
| 935 | OD2 | ASP | A | 458 | 28.108 | 99.939 | 14.217 | 1.00 | 90.84 |
| 936 | N | LYS | A | 459 | 29.196 | 97.451 | 16.076 | 1.00 | 66.05 |
| 937 | CA | LYS | A | 459 | 28.782 | 96.211 | 15.437 | 1.00 | 61.75 |
| 938 | C | LYS | A | 459 | 27.368 | 95.983 | 15.886 | 1.00 | 56.14 |
| 939 | O | LYS | A | 459 | 27.144 | 95.648 | 17.046 | 1.00 | 56.54 |
| 940 | CB | LYS | A | 459 | 29.641 | 95.031 | 15.889 | 1.00 | 68.18 |
| 941 | CG | LYS | A | 459 | 30.801 | 94.804 | 14.969 | 1.00 | 71.85 |
| 942 | CD | LYS | A | 459 | 30.406 | 94.863 | 13.499 | 1.00 | 73.79 |
| 943 | CE | LYS | A | 459 | 31.655 | 94.839 | 12.626 | 1.00 | 76.14 |
| 944 | NZ | LYS | A | 459 | 31.341 | 94.675 | 11.169 | 1.00 | 79.35 |
| 945 | N | ARG | A | 460 | 26.406 | 96.194 | 15.001 | 1.00 | 52.32 |
| 946 | CA | ARG | A | 460 | 25.035 | 95.951 | 15.377 | 1.00 | 47.59 |
| 947 | C | ARG | A | 460 | 24.860 | 94.436 | 15.290 | 1.00 | 43.34 |
| 948 | O | ARG | A | 460 | 25.477 | 93.761 | 14.462 | 1.00 | 40.40 |
| 949 | CB | ARG | A | 460 | 24.104 | 96.726 | 14.456 | 1.00 | 49.27 |
| 950 | CG | ARG | A | 460 | 24.275 | 98.216 | 14.671 | 1.00 | 56.23 |
| 951 | CD | ARG | A | 460 | 23.659 | 99.006 | 13.532 | 1.00 | 63.20 |
| 952 | NE | ARG | A | 460 | 23.997 | 100.426 | 13.596 | 1.00 | 68.02 |
| 953 | CZ | ARG | A | 460 | 25.145 | 100.955 | 13.180 | 1.00 | 70.46 |
| 954 | NH1 | ARG | A | 460 | 26.094 | 100.185 | 12.656 | 1.00 | 72.23 |
| 955 | NH2 | ARG | A | 460 | 25.336 | 102.264 | 13.285 | 1.00 | 73.53 |
| 956 | N | THR | A | 461 | 24.062 | 93.910 | 16.198 | 1.00 | 39.19 |
| 957 | CA | THR | A | 461 | 23.879 | 92.474 | 16.278 | 1.00 | 36.81 |
| 958 | C | THR | A | 461 | 22.447 | 92.004 | 16.401 | 1.00 | 34.03 |
| 959 | O | THR | A | 461 | 21.631 | 92.607 | 17.110 | 1.00 | 33.96 |
| 960 | CB | THR | A | 461 | 24.603 | 91.890 | 17.512 | 1.00 | 39.13 |
| 961 | OG1 | THR | A | 461 | 25.957 | 92.357 | 17.557 | 1.00 | 46.04 |
| 962 | CG2 | THR | A | 461 | 24.630 | 90.380 | 17.421 | 1.00 | 45.48 |
| 963 | N | LEU | A | 462 | 22.157 | 90.904 | 15.714 | 1.00 | 29.84 |
| 964 | CA | LEU | A | 462 | 20.853 | 90.267 | 15.788 | 1.00 | 28.03 |
| 965 | C | LEU | A | 462 | 21.205 | 88.926 | 16.410 | 1.00 | 27.33 |
| 966 | O | LEU | A | 462 | 22.307 | 88.414 | 16.207 | 1.00 | 24.47 |
| 967 | CE | LEU | A | 462 | 20.223 | 90.074 | 14.403 | 1.00 | 25.97 |
| 968 | CG | LEU | A | 462 | 19.888 | 91.362 | 13.635 | 1.00 | 33.39 |
| 969 | CD1 | LEU | A | 462 | 19.172 | 91.007 | 12.319 | 1.00 | 32.36 |
| 970 | CD2 | LEU | A | 462 | 19.014 | 92.263 | 14.483 | 1.00 | 30.72 |
| 971 | N | ALA | A | 463 | 20.286 | 88.359 | 17.178 | 1.00 | 25.83 |
| 972 | CA | ALA | A | 463 | 20.577 | 87.111 | 17.836 | 1.00 | 25.65 |
| 973 | C | ALA | A | 463 | 19.330 | 86.279 | 17.957 | 1.00 | 26.32 |
| 974 | O | ALA | A | 463 | 18.214 | 86.790 | 17.846 | 1.00 | 27.53 |
| 975 | CB | ALA | A | 463 | 21.179 | 87.381 | 19.212 | 1.00 | 28.15 |
| 976 | N | CYS | A | 464 | 19.518 | 84.992 | 18.195 | 1.00 | 21.23 |
| 977 | CA | CYS | A | 464 | 18.388 | 84.093 | 18.276 | 1.00 | 24.77 |
| 978 | C | CYS | A | 464 | 18.599 | 83.016 | 19.314 | 1.00 | 22.14 |
| 979 | O | CYS | A | 464 | 19.683 | 82.431 | 19.402 | 1.00 | 24.66 |
| 980 | CB | CYS | A | 464 | 18.193 | 83.429 | 16.914 | 1.00 | 22.04 |
| 981 | SG | CYS | A | 464 | 16.790 | 82.298 | 16.720 | 1.00 | 34.06 |
| 982 | N | LEU | A | 465 | 17.548 | 82.741 | 20.075 | 1.00 | 22.44 |
| 983 | CA | LEU | A | 465 | 17.598 | 81.697 | 21.089 | 1.00 | 23.11 |
| 984 | C | LEU | A | 465 | 16.644 | 80.615 | 20.641 | 1.00 | 22.43 |
| 985 | O | LEU | A | 465 | 15.483 | 80.898 | 20.352 | 1.00 | 23.73 |
| 986 | CB | LEU | A | 465 | 17.155 | 82.230 | 22.461 | 1.00 | 22.99 |
| 987 | CG | LEU | A | 465 | 16.810 | 81.177 | 23.538 | 1.00 | 19.04 |
| 988 | CD1 | LEU | A | 465 | 18.035 | 80.331 | 23.866 | 1.00 | 21.72 |
| 989 | CD2 | LEU | A | 465 | 16.347 | 81.884 | 24.818 | 1.00 | 28.23 |
| 990 | N | ILE | A | 466 | 17.125 | 79.380 | 20.581 | 1.00 | 22.56 |
| 991 | CA | ILE | A | 466 | 16.277 | 78.268 | 20.169 | 1.00 | 22.11 |
| 992 | C | ILE | A | 466 | 16.302 | 77.256 | 21.297 | 1.00 | 24.25 |
| 993 | O | ILE | A | 466 | 17.375 | 76.815 | 21.706 | 1.00 | 24.93 |
| 994 | CB | ILE | A | 466 | 16.781 | 77.654 | 18.861 | 1.00 | 22.15 |
| 995 | CG1 | TLE | A | 466 | 16.849 | 78.750 | 17.786 | 1.00 | 23.07 |
| 996 | CG2 | ILE | A | 466 | 15.822 | 76.525 | 18.416 | 1.00 | 25.24 |
| 997 | CD1 | ILE | A | 466 | 17.509 | 78.318 | 16.496 | 1.00 | 33.12 |
| 998 | N | GLN | A | 467 | 15.132 | 76.863 | 21.793 | 1.00 | 24.63 |
| 999 | CA | GLN | A | 467 | 15.120 | 75.970 | 22.951 | 1.00 | 28.64 |
| 1000 | C | GLN | A | 467 | 13.982 | 74.963 | 23.072 | 1.00 | 31.08 |
| 1001 | O | GLN | A | 467 | 13.071 | 74.915 | 22.235 | 1.00 | 31.00 |
| 1002 | CB | GLN | A | 467 | 15.172 | 76.830 | 24.228 | 1.00 | 25.58 |
| 1003 | CG | GLN | A | 467 | 13.943 | 77.727 | 24.404 | 1.00 | 25.53 |
| 1004 | CD | GLN | A | 467 | 14.044 | 78.693 | 25.599 | 1.00 | 30.72 |
| 1005 | OE1 | GLN | A | 467 | 14.790 | 78.456 | 26.549 | 1.00 | 29.76 |
| 1006 | NE2 | GLN | A | 467 | 13.273 | 79.779 | 25.550 | 1.00 | 32.18 |
| 1007 | N | ASN | A | 468 | 14.083 | 74.147 | 24.123 | 1.00 | 33.71 |
| 1008 | CA | ASN | A | 468 | 13.114 | 73.106 | 24.459 | 1.00 | 36.96 |
| 1009 | C | ASN | A | 468 | 12.993 | 71.999 | 23.423 | 1.00 | 38.20 |
| 1010 | O | ASN | A | 468 | 11.931 | 71.392 | 23.288 | 1.00 | 38.39 |
| 1011 | CB | ASN | A | 468 | 11.731 | 73.719 | 24.676 | 1.00 | 41.06 |
| 1012 | CG | ASN | A | 468 | 11.748 | 74.837 | 25.686 | 1.00 | 46.41 |
| 1013 | OD1 | ASN | A | 468 | 12.372 | 74.721 | 26.741 | 1.00 | 50.82 |
| 1014 | ND2 | ASN | A | 468 | 11.052 | 75.928 | 25.376 | 1.00 | 53.31 |
| 1015 | N | PHE | A | 469 | 14.065 | 71.732 | 22.685 | 1.00 | 35.74 |
| 1016 | CA | PHE | A | 469 | 14.003 | 70.690 | 21.677 | 1.00 | 33.87 |
| 1017 | C | PHE | A | 469 | 14.804 | 69.453 | 22.056 | 1.00 | 33.63 |
| 1018 | O | PHE | A | 469 | 15.727 | 69.513 | 22.860 | 1.00 | 34.78 |
| 1019 | CB | PHE | A | 469 | 14.495 | 71.228 | 20.322 | 1.00 | 29.12 |
| 1020 | CG | PHE | A | 469 | 15.926 | 71.691 | 20.334 | 1.00 | 26.27 |
| 1021 | CB1 | PHE | A | 469 | 16.959 | 70.792 | 20.133 | 1.00 | 22.45 |
| 1022 | CD2 | PHE | A | 469 | 16.238 | 73.034 | 20.539 | 1.00 | 22.06 |
| 1023 | CE1 | PHE | A | 469 | 18.285 | 71.214 | 20.126 | 1.00 | 23.84 |
| 1024 | CE2 | PHE | A | 469 | 17.564 | 73.465 | 20.536 | 1.00 | 28.02 |
| 1025 | CZ | PHE | A | 469 | 18.593 | 72.549 | 20.327 | 1.00 | 23.33 |
| 1026 | N | MET | A | 470 | 14.431 | 68.327 | 21.466 | 1.00 | 35.64 |
| 1027 | CA | MET | A | 470 | 15.120 | 67.065 | 21.687 | 1.00 | 37.62 |
| 1028 | C | MET | A | 470 | 14.684 | 66.104 | 20.596 | 1.00 | 36.36 |
| 1029 | O | MET | A | 470 | 13.529 | 66.122 | 20.177 | 1.00 | 36.33 |
| 1030 | CB | MET | A | 470 | 14.804 | 66.495 | 23.076 | 1.00 | 42.61 |
| 1031 | CG | MET | A | 470 | 13.354 | 66.179 | 23.350 | 1.00 | 50.60 |
| 1032 | SD | MET | A | 470 | 13.141 | 65.744 | 25.113 | 1.00 | 66.08 |
| 1033 | CE | MET | A | 470 | 13.780 | 64.056 | 25.134 | 1.00 | 66.35 |
| 1034 | N | PRO | A | 471 | 15.613 | 65.283 | 20.079 | 1.00 | 34.63 |
| 1035 | CA | PRO | A | 471 | 17.039 | 65.169 | 20.418 | 1.00 | 32.61 |
| 1036 | C | PRO | A | 471 | 17.818 | 66.453 | 20.143 | 1.00 | 31.92 |
| 1037 | O | PRO | A | 471 | 17.244 | 67.436 | 19.685 | 1.00 | 29.59 |
| 1038 | CE | PRO | A | 471 | 17.520 | 64.015 | 19.538 | 1.00 | 33.95 |
| 1039 | CG | PRO | A | 471 | 16.277 | 63.227 | 19.295 | 1.00 | 37.90 |
| 1040 | CB | PRO | A | 471 | 15.246 | 64.291 | 19.058 | 1.00 | 34.78 |
| 1041 | N | GLU | A | 472 | 19.128 | 66.413 | 20.386 | 1.00 | 32.28 |
| 1042 | CA | GLU | A | 472 | 20.001 | 67.580 | 20.234 | 1.00 | 36.49 |
| 1043 | C | GLU | A | 472 | 20.380 | 68.043 | 18.830 | 1.00 | 35.52 |
| 1044 | O | GLU | A | 472 | 20.870 | 69.162 | 18.683 | 1.00 | 34.87 |
| 1045 | CB | GLU | A | 472 | 21.302 | 67.377 | 21.015 | 1.00 | 41.97 |
| 1046 | CG | GLU | A | 472 | 22.213 | 66.306 | 20.423 | 1.00 | 54.78 |
| 1047 | CD | GLU | A | 472 | 23.527 | 66.144 | 21.184 | 1.00 | 62.41 |
| 1048 | OE1 | GLU | A | 472 | 23.603 | 66.579 | 22.356 | 1.00 | 67.26 |
| 1049 | OE2 | GLU | A | 472 | 24.481 | 65.566 | 20.613 | 1.00 | 64.96 |
| 1050 | N | ASP | A | 473 | 20.161 | 67.221 | 17.805 | 1.00 | 34.38 |
| 1051 | CA | ASP | A | 473 | 20.543 | 67.629 | 16.452 | 1.00 | 31.83 |
| 1052 | C | ASP | A | 473 | 19.602 | 68.686 | 15.886 | 1.00 | 31.24 |
| 1053 | O | ASP | A | 473 | 18.380 | 68.528 | 15.901 | 1.00 | 31.80 |
| 1054 | CB | ASP | A | 473 | 20.621 | 66.420 | 15.516 | 1.00 | 34.73 |
| 1055 | CG | ASP | A | 473 | 21.746 | 65.453 | 15.904 | 1.00 | 40.83 |
| 1056 | OD1 | ASP | A | 473 | 22.841 | 65.924 | 16.296 | 1.00 | 44.18 |
| 1057 | OD2 | ASP | A | 473 | 21.539 | 64.222 | 15.810 | 1.00 | 40.17 |
| 1058 | N | ILE | A | 474 | 20.184 | 69.774 | 15.398 | 1.00 | 26.96 |
| 1059 | CA | ILE | A | 474 | 19.393 | 70.856 | 14.854 | 1.00 | 25.39 |
| 1060 | C | ILE | A | 474 | 20.225 | 71.715 | 13.892 | 1.00 | 24.74 |
| 1061 | O | ILE | A | 474 | 21.436 | 71.824 | 14.031 | 1.00 | 25.20 |
| 1062 | CB | ILE | A | 474 | 18.842 | 71.738 | 16.008 | 1.00 | 23.81 |
| 1063 | CG1 | ILE | A | 474 | 17.748 | 72.675 | 15.502 | 1.00 | 22.23 |
| 1064 | CG2 | ILE | A | 474 | 19.974 | 72.538 | 16.647 | 1.00 | 27.10 |
| 1065 | CD1 | ILE | A | 474 | 17.064 | 73.456 | 16.650 | 1.00 | 25.30 |
| 1066 | N | SER | A | 475 | 19.570 | 72.288 | 12.891 | 1.00 | 23.27 |
| 1067 | CA | SER | A | 475 | 20.252 | 73.170 | 11.952 | 1.00 | 22.17 |
| 1068 | C | SER | A | 475 | 19.609 | 74.534 | 12.148 | 1.00 | 21.71 |
| 1069 | O | SER | A | 475 | 18.393 | 74.649 | 12.224 | 1.00 | 20.84 |
| 1070 | CB | SER | A | 475 | 20.049 | 72.721 | 10.507 | 1.00 | 20.43 |
| 1071 | OG | SER | A | 475 | 20.589 | 71.436 | 10.294 | 1.00 | 30.00 |
| 1072 | N | VAL | A | 476 | 20.442 | 75.558 | 12.213 | 1.00 | 21.76 |
| 1073 | CA | VAL | A | 476 | 19.980 | 76.906 | 12.425 | 1.00 | 21.72 |
| 1074 | C | VAL | A | 476 | 20.435 | 77.725 | 11.249 | 1.00 | 23.56 |
| 1075 | O | VAL | A | 476 | 21.555 | 77.564 | 10.780 | 1.00 | 25.95 |
| 1076 | CB | VAL | A | 476 | 20.606 | 77.513 | 13.715 | 1.00 | 21.56 |
| 1077 | CG1 | VAL | A | 476 | 20.157 | 78.975 | 13.878 | 1.00 | 18.70 |
| 1078 | CG2 | VAL | A | 476 | 20.197 | 76.685 | 14.934 | 1.00 | 20.99 |
| 1079 | N | GLN | A | 477 | 19.574 | 78.606 | 10.764 | 1.00 | 25.16 |
| 1080 | CA | GLN | A | 477 | 19.965 | 79.428 | 9.640 | 1.00 | 26.65 |
| 1081 | C | GLN | A | 477 | 19.263 | 80.767 | 9.662 | 1.00 | 26.62 |
| 1082 | O | GLN | A | 477 | 18.160 | 80.891 | 10.175 | 1.00 | 26.40 |
| 1083 | CB | GLN | A | 477 | 19.701 | 78.683 | 8.325 | 1.00 | 31.51 |
| 1084 | CG | GLN | A | 477 | 18.289 | 78.252 | 8.089 | 1.00 | 44.27 |
| 1085 | CB | GLN | A | 477 | 18.188 | 77.095 | 7.084 | 1.00 | 47.76 |
| 1086 | OE1 | GLN | A | 477 | 17.104 | 76.786 | 6.598 | 1.00 | 50.57 |
| 1087 | NE2 | GLN | A | 477 | 19.317 | 76.449 | 6.787 | 1.00 | 47.32 |
| 1088 | N | THR | A | 478 | 19.941 | 81.780 | 9.146 | 1.00 | 27.41 |
| 1089 | CA | THR | A | 478 | 19.375 | 83.112 | 9.074 | 1.00 | 28.36 |
| 1090 | C | THR | A | 478 | 18.944 | 83.408 | 7.635 | 1.00 | 30.10 |
| 1091 | O | THR | A | 478 | 19.567 | 82.940 | 6.672 | 1.00 | 25.31 |
| 1092 | CB | THR | A | 478 | 20.404 | 84.139 | 9.533 | 1.00 | 28.76 |
| 1093 | CG | THR | A | 478 | 20.649 | 84.098 | 11.013 | 1.00 | 34.11 |
| 1094 | CD1 | THR | A | 478 | 21.522 | 83.287 | 11.685 | 1.00 | 31.25 |
| 1095 | CD2 | THR | A | 478 | 19.980 | 84.878 | 12.006 | 1.00 | 31.01 |
| 1096 | NE1 | THR | A | 478 | 21.436 | 83.515 | 13.031 | 1.00 | 27.71 |
| 1097 | CE2 | THR | A | 478 | 20.496 | 84.487 | 13.259 | 1.00 | 31.95 |
| 1098 | CE3 | THR | A | 478 | 18.993 | 85.871 | 11.960 | 1.00 | 34.16 |
| 1099 | CZ2 | THR | A | 478 | 20.055 | 85.058 | 14.463 | 1.00 | 25.26 |
| 1100 | CZ3 | THR | A | 478 | 18.556 | 86.439 | 13.153 | 1.00 | 31.64 |
| 1101 | CH2 | THR | A | 478 | 19.089 | 86.029 | 14.387 | 1.00 | 28.89 |
| 1102 | N | LEU | A | 479 | 17.877 | 84.182 | 7.498 | 1.00 | 32.48 |
| 1103 | CA | LEU | A | 479 | 17.355 | 84.551 | 6.187 | 1.00 | 37.36 |
| 1104 | C | LEU | A | 479 | 16.943 | 86.016 | 6.140 | 1.00 | 40.42 |
| 1105 | O | LEU | A | 479 | 16.448 | 86.560 | 7.123 | 1.00 | 40.14 |
| 1106 | CB | LEU | A | 479 | 16.122 | 83.722 | 5.848 | 1.00 | 39.83 |
| 1107 | CG | LEU | A | 479 | 16.044 | 82.254 | 6.258 | 1.00 | 44.57 |
| 1108 | CD1 | LEU | A | 479 | 14.713 | 81.710 | 5.810 | 1.00 | 52.98 |
| 1109 | CD2 | LEU | A | 479 | 17.161 | 81.470 | 5.634 | 1.00 | 50.71 |
| 1110 | N | HIS | A | 480 | 17.152 | 86.653 | 4.998 | 1.00 | 43.05 |
| 1111 | CA | HIS | A | 480 | 16.723 | 88.028 | 4.824 | 1.00 | 49.20 |
| 1112 | C | HIS | A | 480 | 15.464 | 87.854 | 3.984 | 1.00 | 52.73 |
| 1113 | O | HIS | A | 480 | 15.509 | 87.895 | 2.753 | 1.00 | 52.90 |
| 1114 | CB | HIS | A | 480 | 17.756 | 88.846 | 4.057 | 1.00 | 52.25 |
| 1115 | CG | HIS | A | 480 | 17.330 | 90.259 | 3.814 | 1.00 | 57.22 |
| 1116 | ND1 | HIS | A | 480 | 17.525 | 91.263 | 4.736 | 1.00 | 61.37 |
| 1117 | CD2 | HIS | A | 480 | 16.658 | 90.822 | 2.781 | 1.00 | 59.16 |
| 1118 | CE1 | HIS | A | 480 | 16.993 | 92.384 | 4.285 | 1.00 | 59.88 |
| 1119 | NE2 | HIS | A | 480 | 16.460 | 92.144 | 3.100 | 1.00 | 66.11 |
| 1120 | N | ASN | A | 481 | 14.347 | 87.629 | 4.670 | 1.00 | 56.65 |
| 1121 | CA | ASN | A | 481 | 13.057 | 87.394 | 4.030 | 1.00 | 59.93 |
| 1122 | C | ASN | A | 481 | 13.009 | 85.940 | 3.581 | 1.00 | 59.65 |
| 1123 | O | ASN | A | 481 | 13.031 | 85.029 | 4.405 | 1.00 | 61.04 |
| 1124 | CB | ASN | A | 481 | 12.849 | 88.312 | 2.818 | 1.00 | 64.65 |
| 1125 | CG | ASN | A | 481 | 12.578 | 89.748 | 3.210 | 1.00 | 72.38 |
| 1126 | OD1 | ASN | A | 481 | 11.636 | 90.037 | 3.953 | 1.00 | 74.96 |
| 1127 | ND2 | ASN | A | 481 | 13.401 | 90.662 | 2.707 | 1.00 | 75.60 |
| 1128 | N | GLU | A | 482 | 12.975 | 85.728 | 2.273 | 1.00 | 58.97 |
| 1129 | CA | GLU | A | 482 | 12.903 | 84.387 | 1.704 | 1.00 | 58.55 |
| 1130 | C | GLU | A | 482 | 14.254 | 83.785 | 1.318 | 1.00 | 55.30 |
| 1131 | O | GLU | A | 482 | 14.347 | 82.587 | 1.056 | 1.00 | 57.27 |
| 1132 | CB | GLU | A | 482 | 11.993 | 84.424 | 0.473 | 1.00 | 65.42 |
| 1133 | CG | GLU | A | 482 | 11.787 | 85.840 | −0.072 | 1.00 | 73.94 |
| 1134 | CB | GLU | A | 482 | 11.127 | 85.865 | −1.432 | 1.00 | 80.29 |
| 1135 | OE1 | GLU | A | 482 | 11.770 | 85.432 | −2.413 | 1.00 | 86.31 |
| 1136 | OE2 | GLU | A | 482 | 9.967 | 86.316 | −1.520 | 1.00 | 82.73 |
| 1137 | N | VAL | A | 483 | 15.299 | 84.603 | 1.286 | 1.00 | 50.25 |
| 1138 | CA | VAL | A | 483 | 16.612 | 84.113 | 0.889 | 1.00 | 45.78 |
| 1139 | C | VAL | A | 483 | 17.575 | 83.867 | 2.048 | 1.00 | 43.04 |
| 1140 | O | VAL | A | 483 | 17.766 | 84.720 | 2.902 | 1.00 | 41.34 |
| 1141 | CB | VAL | A | 483 | 17.261 | 85.073 | −0.133 | 1.00 | 43.76 |
| 1142 | CG1 | VAL | A | 483 | 17.317 | 86.471 | 0.432 | 1.00 | 46.25 |
| 1143 | CG2 | VAL | A | 483 | 18.654 | 84.589 | −0.494 | 1.00 | 41.37 |
| 1144 | N | GLN | A | 484 | 18.188 | 82.688 | 2.050 | 1.00 | 42.30 |
| 1145 | CA | GLN | A | 484 | 19.126 | 82.300 | 3.095 | 1.00 | 43.21 |
| 1146 | C | GLN | A | 484 | 20.509 | 82.942 | 2.959 | 1.00 | 42.48 |
| 1147 | O | GLN | A | 484 | 21.055 | 83.062 | 1.856 | 1.00 | 41.64 |
| 1148 | CB | GLN | A | 484 | 19.269 | 80.774 | 3.129 | 1.00 | 45.96 |
| 1149 | CG | GLN | A | 484 | 20.331 | 80.276 | 4.101 | 1.00 | 54.43 |
| 1150 | CD | GLN | A | 484 | 20.427 | 78.760 | 4.159 | 1.00 | 59.02 |
| 1151 | OE1 | GLN | A | 484 | 21.400 | 78.209 | 4.677 | 1.00 | 59.71 |
| 1152 | NE2 | GLN | A | 484 | 19.410 | 78.078 | 3.636 | 1.00 | 61.46 |
| 1153 | N | LEU | A | 485 | 21.061 | 83.361 | 4.094 | 1.00 | 38.99 |
| 1154 | CA | LEU | A | 485 | 22.382 | 83.982 | 4.147 | 1.00 | 39.17 |
| 1155 | C | LEU | A | 485 | 23.458 | 82.900 | 4.247 | 1.00 | 40.35 |
| 1156 | O | LEU | A | 485 | 23.183 | 81.778 | 4.670 | 1.00 | 37.39 |
| 1157 | CB | LEU | A | 485 | 22.493 | 84.895 | 5.375 | 1.00 | 37.16 |
| 1158 | CG | LEU | A | 485 | 21.460 | 86.021 | 5.498 | 1.00 | 39.66 |
| 1159 | CD1 | LEU | A | 485 | 21.670 | 86.764 | 6.807 | 1.00 | 39.30 |
| 1160 | CD2 | LEU | A | 485 | 21.580 | 86.956 | 4.305 | 1.00 | 40.56 |
| 1161 | N | PRO | A | 486 | 24.700 | 83.227 | 3.858 | 1.00 | 42.55 |
| 1162 | CA | PRO | A | 486 | 25.772 | 82.234 | 3.942 | 1.00 | 46.30 |
| 1163 | C | PRO | A | 486 | 25.940 | 81.850 | 5.412 | 1.00 | 49.32 |
| 1164 | O | PRO | A | 486 | 25.748 | 82.684 | 6.294 | 1.00 | 48.81 |
| 1165 | CB | PRO | A | 486 | 26.991 | 82.994 | 3.425 | 1.00 | 45.64 |
| 1166 | CG | PRO | A | 486 | 26.413 | 84.068 | 2.572 | 1.00 | 45.29 |
| 1167 | CD | PRO | A | 486 | 25.206 | 84.505 | 3.331 | 1.00 | 40.65 |
| 1168 | N | ASP | A | 487 | 26.310 | 80.603 | 5.671 | 1.00 | 53.39 |
| 1169 | CA | ASP | A | 487 | 26.507 | 80.135 | 7.038 | 1.00 | 57.75 |
| 1170 | C | ASP | A | 487 | 27.603 | 80.940 | 7.750 | 1.00 | 57.25 |
| 1171 | O | ASP | A | 487 | 27.424 | 81.395 | 8.887 | 1.00 | 57.75 |
| 1172 | CB | ASP | A | 487 | 26.892 | 78.656 | 7.023 | 1.00 | 67.82 |
| 1173 | CG | ASP | A | 487 | 26.821 | 78.023 | 8.397 | 1.00 | 76.17 |
| 1174 | OD1 | ASP | A | 487 | 27.291 | 78.655 | 9.366 | 1.00 | 84.77 |
| 1175 | OD2 | ASP | A | 487 | 26.303 | 76.889 | 8.508 | 1.00 | 86.94 |
| 1176 | N | ALA | A | 488 | 28.727 | 81.122 | 7.065 | 1.00 | 54.64 |
| 1177 | CA | ALA | A | 488 | 29.871 | 81.848 | 7.607 | 1.00 | 53.45 |
| 1178 | C | ALA | A | 488 | 29.531 | 83.179 | 8.263 | 1.00 | 52.74 |
| 1179 | O | ALA | A | 488 | 30.359 | 83.757 | 8.966 | 1.00 | 53.06 |
| 1180 | CB | ALA | A | 488 | 30.892 | 82.070 | 6.515 | 1.00 | 53.75 |
| 1181 | N | ARG | A | 489 | 28.313 | 83.662 | 8.044 | 1.00 | 50.96 |
| 1182 | CA | ARG | A | 489 | 27.896 | 84.940 | 8.609 | 1.00 | 47.84 |
| 1183 | C | ARG | A | 489 | 27.436 | 84.895 | 10.064 | 1.00 | 45.10 |
| 1184 | O | ARG | A | 489 | 27.442 | 85.919 | 10.737 | 1.00 | 43.37 |
| 1185 | CB | ARG | A | 489 | 26.792 | 85.554 | 7.746 | 1.00 | 48.99 |
| 1186 | CG | ARG | A | 489 | 27.306 | 86.368 | 6.576 | 1.00 | 50.01 |
| 1187 | CD | ARG | A | 489 | 27.173 | 87.839 | 6.873 | 1.00 | 50.36 |
| 1188 | NE | ARG | A | 489 | 26.019 | 88.413 | 6.195 | 1.00 | 56.65 |
| 1189 | CZ | ARG | A | 489 | 25.434 | 89.552 | 6.543 | 1.00 | 58.23 |
| 1190 | NH1 | ARG | A | 489 | 25.886 | 90.248 | 7.578 | 1.00 | 61.07 |
| 1191 | NH2 | ARG | A | 489 | 24.407 | 90.006 | 5.840 | 1.00 | 59.63 |
| 1192 | N | HIS | A | 490 | 27.027 | 83.728 | 10.549 | 1.00 | 42.41 |
| 1193 | CA | HIS | A | 490 | 26.576 | 83.635 | 11.934 | 1.00 | 41.63 |
| 1194 | C | HIS | A | 490 | 27.390 | 82.648 | 12.766 | 1.00 | 39.71 |
| 1195 | O | HIS | A | 490 | 28.129 | 81.832 | 12.235 | 1.00 | 41.30 |
| 1196 | CB | HIS | A | 490 | 25.092 | 83.252 | 12.000 | 1.00 | 42.40 |
| 1197 | CG | HIS | A | 490 | 24.793 | 81.880 | 11.486 | 1.00 | 43.27 |
| 1198 | ND1 | HIS | A | 490 | 24.423 | 81.634 | 10.181 | 1.00 | 42.27 |
| 1199 | CD2 | HIS | A | 490 | 24.821 | 80.674 | 12.101 | 1.00 | 44.41 |
| 1200 | CE1 | HIS | A | 490 | 24.236 | 80.338 | 10.014 | 1.00 | 39.88 |
| 1201 | NE2 | HIS | A | 490 | 24.470 | 79.732 | 11.166 | 1.00 | 45.37 |
| 1202 | N | SER | A | 491 | 27.245 | 82.742 | 14.080 | 1.00 | 37.20 |
| 1203 | CA | SER | A | 491 | 27.946 | 81.866 | 15.005 | 1.00 | 36.36 |
| 1204 | C | SER | A | 491 | 26.915 | 81.199 | 15.927 | 1.00 | 33.06 |
| 1205 | O | SER | A | 491 | 26.172 | 81.879 | 16.639 | 1.00 | 33.77 |
| 1206 | CB | SER | A | 491 | 28.949 | 82.690 | 15.812 | 1.00 | 35.42 |
| 1207 | OG | SER | A | 491 | 29.547 | 81.906 | 16.818 | 1.00 | 43.65 |
| 1208 | N | THR | A | 492 | 26.878 | 79.872 | 15.912 | 1.00 | 29.90 |
| 1209 | CA | THR | A | 492 | 25.927 | 79.105 | 16.717 | 1.00 | 29.91 |
| 1210 | C | THR | A | 492 | 26.623 | 78.278 | 17.813 | 1.00 | 29.20 |
| 1211 | O | THR | A | 492 | 27.609 | 77.604 | 17.548 | 1.00 | 30.85 |
| 1212 | CB | THR | A | 492 | 25.094 | 78.153 | 15.802 | 1.00 | 29.85 |
| 1213 | OG1 | THR | A | 492 | 24.403 | 78.930 | 14.819 | 1.00 | 35.86 |
| 1214 | CG2 | THR | A | 492 | 24.059 | 77.366 | 16.602 | 1.00 | 28.05 |
| 1215 | N | THR | A | 493 | 26.095 | 78.319 | 19.035 | 1.00 | 29.49 |
| 1216 | CA | THR | A | 493 | 26.687 | 77.568 | 20.143 | 1.00 | 30.19 |
| 1217 | C | THR | A | 493 | 26.305 | 76.099 | 20.044 | 1.00 | 32.39 |
| 1218 | O | THR | A | 493 | 25.390 | 75.737 | 19.314 | 1.00 | 30.70 |
| 1219 | CB | THR | A | 493 | 26.222 | 78.098 | 21.532 | 1.00 | 27.82 |
| 1220 | OG1 | THR | A | 493 | 24.794 | 78.008 | 21.640 | 1.00 | 30.48 |
| 1221 | CG2 | THR | A | 493 | 26.647 | 79.533 | 21.728 | 1.00 | 22.03 |
| 1222 | N | GLN | A | 494 | 27.014 | 75.253 | 20.778 | 1.00 | 35.09 |
| 1223 | CA | GLN | A | 494 | 26.721 | 73.821 | 20.770 | 1.00 | 38.19 |
| 1224 | C | GLN | A | 494 | 25.509 | 73.568 | 21.651 | 1.00 | 36.48 |
| 1225 | O | GLN | A | 494 | 25.330 | 74.240 | 22.668 | 1.00 | 35.75 |
| 1226 | CB | GLN | A | 494 | 27.904 | 73.019 | 21.325 | 1.00 | 43.09 |
| 1227 | CG | GLN | A | 494 | 29.167 | 73.098 | 20.490 | 1.00 | 58.35 |
| 1228 | CB | GLN | A | 494 | 28.934 | 72.671 | 19.058 | 1.00 | 65.11 |
| 1229 | OE1 | GLN | A | 494 | 28.391 | 71.597 | 18.797 | 1.00 | 71.67 |
| 1230 | NE2 | GLN | A | 494 | 29.345 | 73.512 | 18.118 | 1.00 | 71.87 |
| 1231 | N | PRO | A | 495 | 24.661 | 72.598 | 21.272 | 1.00 | 36.89 |
| 1232 | CA | PRO | A | 495 | 23.470 | 72.278 | 22.063 | 1.00 | 38.37 |
| 1233 | C | PRO | A | 495 | 23.863 | 71.991 | 23.513 | 1.00 | 40.80 |
| 1234 | O | PRO | A | 495 | 24.837 | 71.286 | 23.773 | 1.00 | 40.76 |
| 1235 | CB | PRO | A | 495 | 22.920 | 71.043 | 21.371 | 1.00 | 37.83 |
| 1236 | CG | PRO | A | 495 | 23.300 | 71.285 | 19.926 | 1.00 | 40.41 |
| 1237 | CD | PRO | A | 495 | 24.716 | 71.791 | 20.039 | 1.00 | 35.81 |
| 1238 | N | ARG | A | 496 | 23.120 | 72.560 | 24.450 | 1.00 | 42.05 |
| 1239 | CA | ARG | A | 496 | 23.392 | 72.345 | 25.860 | 1.00 | 47.69 |
| 1240 | C | ARG | A | 496 | 22.146 | 71.828 | 26.558 | 1.00 | 51.66 |
| 1241 | O | ARG | A | 496 | 21.042 | 72.317 | 26.316 | 1.00 | 51.95 |
| 1242 | CB | ARG | A | 496 | 23.849 | 73.646 | 26.516 | 1.00 | 45.81 |
| 1243 | CG | ARG | A | 496 | 25.283 | 74.001 | 26.208 | 1.00 | 51.53 |
| 1244 | CD | ARG | A | 496 | 25.653 | 75.383 | 26.709 | 1.00 | 53.72 |
| 1245 | NE | ARG | A | 496 | 27.046 | 75.425 | 27.137 | 1.00 | 57.50 |
| 1246 | CZ | ARG | A | 496 | 27.478 | 74.992 | 28.317 | 1.00 | 58.59 |
| 1247 | NH1 | ARG | A | 496 | 26.626 | 74.489 | 29.199 | 1.00 | 57.22 |
| 1248 | NH2 | ARG | A | 496 | 28.768 | 75.058 | 28.612 | 1.00 | 65.31 |
| 1249 | N | LYS | A | 497 | 22.319 | 70.827 | 27.413 | 1.00 | 56.98 |
| 1250 | CA | LYS | A | 497 | 21.192 | 70.265 | 28.147 | 1.00 | 61.91 |
| 1251 | C | LYS | A | 497 | 20.733 | 71.299 | 29.145 | 1.00 | 63.97 |
| 1252 | O | LYS | A | 497 | 21.405 | 72.300 | 29.371 | 1.00 | 64.61 |
| 1253 | CB | LYS | A | 497 | 21.597 | 69.013 | 28.928 | 1.00 | 66.35 |
| 1254 | CG | LYS | A | 497 | 21.819 | 67.752 | 28.113 | 1.00 | 74.14 |
| 1255 | CD | LYS | A | 497 | 22.241 | 66.607 | 29.034 | 1.00 | 80.69 |
| 1256 | CE | LYS | A | 497 | 22.408 | 65.296 | 28.286 | 1.00 | 84.48 |
| 1257 | NZ | LYS | A | 497 | 22.861 | 64.215 | 29.211 | 1.00 | 88.26 |
| 1258 | N | THR | A | 498 | 19.583 | 71.045 | 29.748 | 1.00 | 67.67 |
| 1259 | CA | THR | A | 498 | 19.028 | 71.931 | 30.758 | 1.00 | 71.26 |
| 1260 | C | THR | A | 498 | 18.316 | 71.050 | 31.760 | 1.00 | 73.04 |
| 1261 | O | THR | A | 498 | 18.610 | 69.857 | 31.864 | 1.00 | 72.83 |
| 1262 | CB | THR | A | 498 | 18.015 | 72.921 | 30.155 | 1.00 | 71.80 |
| 1263 | OG1 | THR | A | 498 | 17.045 | 72.208 | 29.379 | 1.00 | 71.58 |
| 1264 | CG2 | THR | A | 498 | 18.722 | 73.930 | 29.281 | 1.00 | 73.61 |
| 1265 | N | LYS | A | 499 | 17.380 | 71.638 | 32.495 | 1.00 | 75.57 |
| 1266 | CA | LYS | A | 499 | 16.607 | 70.895 | 33.480 | 1.00 | 77.62 |
| 1267 | C | LYS | A | 499 | 15.458 | 70.173 | 32.779 | 1.00 | 77.92 |
| 1268 | O | LYS | A | 499 | 14.504 | 70.809 | 32.332 | 1.00 | 79.19 |
| 1269 | CB | LYS | A | 499 | 16.042 | 71.848 | 34.533 | 1.00 | 80.22 |
| 1270 | CG | LYS | A | 499 | 17.069 | 72.428 | 35.496 | 1.00 | 81.04 |
| 1271 | CB | LYS | A | 499 | 17.507 | 71.397 | 36.529 | 1.00 | 83.26 |
| 1272 | CE | LYS | A | 499 | 18.368 | 72.031 | 37.613 | 1.00 | 84.81 |
| 1273 | NZ | LYS | A | 499 | 18.736 | 71.055 | 38.675 | 1.00 | 86.04 |
| 1274 | N | GLY | A | 500 | 15.566 | 68.852 | 32.667 | 1.00 | 76.91 |
| 1275 | CA | GLY | A | 500 | 14.523 | 68.056 | 32.039 | 1.00 | 75.83 |
| 1276 | C | GLY | A | 500 | 14.040 | 68.459 | 30.655 | 1.00 | 75.14 |
| 1277 | O | GLY | A | 500 | 14.024 | 67.629 | 29.745 | 1.00 | 75.52 |
| 1278 | N | SER | A | 501 | 13.642 | 69.718 | 30.495 | 1.00 | 73.08 |
| 1279 | CA | SER | A | 501 | 13.132 | 70.230 | 29.225 | 1.00 | 71.65 |
| 1280 | C | SER | A | 501 | 13.764 | 69.592 | 27.994 | 1.00 | 68.64 |
| 1281 | O | SER | A | 501 | 13.085 | 68.910 | 27.223 | 1.00 | 71.13 |
| 1282 | CB | SER | A | 501 | 13.321 | 71.746 | 29.147 | 1.00 | 74.43 |
| 1283 | OG | SER | A | 501 | 14.667 | 72.076 | 28.859 | 1.00 | 80.92 |
| 1284 | N | GLY | A | 502 | 15.060 | 69.814 | 27.810 | 1.00 | 63.14 |
| 1285 | CA | GLY | A | 502 | 15.741 | 69.256 | 26.657 | 1.00 | 54.62 |
| 1286 | C | GLY | A | 502 | 16.982 | 70.048 | 26.297 | 1.00 | 48.04 |
| 1287 | O | GLY | A | 502 | 17.917 | 70.131 | 27.094 | 1.00 | 48.08 |
| 1288 | N | PHE | A | 503 | 17.001 | 70.642 | 25.106 | 1.00 | 40.50 |
| 1289 | CA | PHE | A | 503 | 18.173 | 71.411 | 24.692 | 1.00 | 33.81 |
| 1290 | C | PHE | A | 503 | 17.908 | 72.827 | 24.212 | 1.00 | 28.39 |
| 1291 | O | PHE | A | 503 | 16.789 | 73.192 | 23.856 | 1.00 | 26.35 |
| 1292 | CB | PHE | A | 503 | 18.938 | 70.681 | 23.583 | 1.00 | 35.09 |
| 1293 | CG | PHE | A | 503 | 19.452 | 69.337 | 23.981 | 1.00 | 34.16 |
| 1294 | CD1 | PHE | A | 503 | 18.630 | 68.219 | 23.926 | 1.00 | 35.05 |
| 1295 | CD2 | PHE | A | 503 | 20.755 | 69.189 | 24.430 | 1.00 | 35.90 |
| 1296 | CE1 | PHE | A | 503 | 19.099 | 66.967 | 24.317 | 1.00 | 36.00 |
| 1297 | CE2 | PHE | A | 503 | 21.238 | 67.944 | 24.826 | 1.00 | 40.04 |
| 1298 | CZ | PHE | A | 503 | 20.408 | 66.829 | 24.771 | 1.00 | 38.82 |
| 1299 | N | PHE | A | 504 | 18.967 | 73.625 | 24.216 | 1.00 | 26.16 |
| 1300 | CA | PHE | A | 504 | 18.891 | 74.988 | 23.731 | 1.00 | 25.00 |
| 1301 | C | PHE | A | 504 | 20.182 | 75.314 | 23.004 | 1.00 | 26.57 |
| 1302 | O | PHE | A | 504 | 21.221 | 74.708 | 23.267 | 1.00 | 26.73 |
| 1303 | CB | PHE | A | 504 | 18.640 | 75.984 | 24.858 | 1.00 | 22.46 |
| 1304 | CG | PHE | A | 504 | 19.834 | 76.270 | 25.718 | 1.00 | 27.70 |
| 1305 | CD1 | PHE | A | 504 | 20.674 | 77.340 | 25.433 | 1.00 | 28.94 |
| 1306 | CD2 | PHE | A | 504 | 20.083 | 75.514 | 26.857 | 1.00 | 31.34 |
| 1307 | CE1 | PHE | A | 504 | 21.743 | 77.662 | 26.273 | 1.00 | 30.08 |
| 1308 | CE2 | PHE | A | 504 | 21.151 | 75.828 | 27.704 | 1.00 | 32.65 |
| 1309 | CZ | PHE | A | 504 | 21.979 | 76.909 | 27.403 | 1.00 | 32.80 |
| 1310 | N | VAL | A | 505 | 20.091 | 76.270 | 22.085 | 1.00 | 23.68 |
| 1311 | CA | VAL | A | 505 | 21.211 | 76.705 | 21.286 | 1.00 | 24.67 |
| 1312 | C | VAL | A | 505 | 21.041 | 78.215 | 21.072 | 1.00 | 23.86 |
| 1313 | O | VAL | A | 505 | 19.928 | 78.725 | 21.049 | 1.00 | 21.24 |
| 1314 | CB | VAL | A | 505 | 21.208 | 75.926 | 19.939 | 1.00 | 27.95 |
| 1315 | CG1 | VAL | A | 505 | 20.134 | 76.464 | 19.020 | 1.00 | 25.61 |
| 1316 | CG2 | VAL | A | 505 | 22.558 | 75.957 | 19.314 | 1.00 | 33.51 |
| 1317 | N | PHE | A | 506 | 22.146 | 78.936 | 20.959 | 1.00 | 26.20 |
| 1318 | CA | PHE | A | 506 | 22.086 | 80.382 | 20.754 | 1.00 | 26.10 |
| 1319 | C | PHE | A | 506 | 22.846 | 80.745 | 19.479 | 1.00 | 24.83 |
| 1320 | O | PHE | A | 506 | 23.928 | 80.231 | 19.241 | 1.00 | 25.52 |
| 1321 | CB | PHE | A | 506 | 22.725 | 81.104 | 21.939 | 1.00 | 28.18 |
| 1322 | CG | PHE | A | 506 | 22.758 | 82.593 | 21.797 | 1.00 | 35.33 |
| 1323 | CD1 | PHE | A | 506 | 21.620 | 83.355 | 22.020 | 1.00 | 42.56 |
| 1324 | CD2 | PHE | A | 506 | 23.937 | 83.241 | 21.443 | 1.00 | 40.04 |
| 1325 | CE1 | PHE | A | 506 | 21.660 | 84.746 | 21.892 | 1.00 | 44.70 |
| 1326 | CE2 | PHE | A | 506 | 23.987 | 84.623 | 21.311 | 1.00 | 40.54 |
| 1327 | CZ | PHE | A | 506 | 22.851 | 85.377 | 21.536 | 1.00 | 42.12 |
| 1328 | N | SER | A | 507 | 22.290 | 81.639 | 18.673 | 1.00 | 24.69 |
| 1329 | CA | SER | A | 507 | 22.941 | 82.050 | 17.426 | 1.00 | 24.73 |
| 1330 | C | SER | A | 507 | 23.065 | 83.578 | 17.355 | 1.00 | 26.40 |
| 1331 | O | SER | A | 507 | 22.113 | 84.292 | 17.609 | 1.00 | 25.99 |
| 1332 | CB | SER | A | 507 | 22.151 | 81.521 | 16.221 | 1.00 | 22.19 |
| 1333 | OG | SER | A | 507 | 22.740 | 81.947 | 15.009 | 1.00 | 30.30 |
| 1334 | N | ARG | A | 508 | 24.255 | 84.062 | 17.009 | 1.00 | 26.98 |
| 1335 | CA | ARG | A | 508 | 24.545 | 85.496 | 16.913 | 1.00 | 25.95 |
| 1336 | C | ARG | A | 508 | 24.851 | 85.911 | 15.463 | 1.00 | 24.97 |
| 1337 | O | ARG | A | 508 | 25.621 | 85.250 | 14.785 | 1.00 | 24.35 |
| 1338 | CB | ARG | A | 508 | 25.755 | 85.801 | 17.790 | 1.00 | 21.97 |
| 1339 | CG | ARG | A | 508 | 26.249 | 87.235 | 17.756 | 1.00 | 25.32 |
| 1340 | CD | ARG | A | 508 | 27.474 | 87.372 | 18.656 | 1.00 | 25.67 |
| 1341 | NE | ARG | A | 508 | 27.898 | 88.758 | 18.788 | 1.00 | 36.00 |
| 1342 | CZ | ARG | A | 508 | 28.757 | 89.357 | 17.972 | 1.00 | 36.06 |
| 1343 | NH1 | ARG | A | 508 | 29.293 | 88.682 | 16.963 | 1.00 | 34.19 |
| 1344 | NH2 | ARG | A | 508 | 29.062 | 90.633 | 18.157 | 1.00 | 34.70 |
| 1345 | N | LEU | A | 509 | 24.269 | 87.017 | 15.001 | 1.00 | 26.44 |
| 1346 | CA | LEU | A | 509 | 24.492 | 87.479 | 13.621 | 1.00 | 28.45 |
| 1347 | C | LEU | A | 509 | 24.794 | 88.973 | 13.540 | 1.00 | 28.87 |
| 1348 | O | LEU | A | 509 | 23.893 | 89.797 | 13.699 | 1.00 | 30.99 |
| 1349 | CB | LEU | A | 509 | 23.259 | 87.177 | 12.758 | 1.00 | 27.28 |
| 1350 | CG | LEU | A | 509 | 23.270 | 87.733 | 11.321 | 1.00 | 31.90 |
| 1351 | CD1 | LEU | A | 509 | 24.284 | 86.985 | 10.453 | 1.00 | 29.48 |
| 1352 | CD2 | LEU | A | 509 | 21.886 | 87.599 | 10.732 | 1.00 | 28.89 |
| 1353 | N | GLU | A | 510 | 26.054 | 89.323 | 13.288 | 1.00 | 31.92 |
| 1354 | CA | GLU | A | 510 | 26.448 | 90.728 | 13.189 | 1.00 | 34.67 |
| 1355 | C | GLU | A | 510 | 26.005 | 91.290 | 11.845 | 1.00 | 36.35 |
| 1356 | O | GLU | A | 510 | 26.199 | 90.652 | 10.812 | 1.00 | 34.66 |
| 1357 | CB | GLU | A | 510 | 27.970 | 90.875 | 13.343 | 1.00 | 41.07 |
| 1358 | CG | GLU | A | 510 | 28.510 | 90.383 | 14.696 | 1.00 | 54.35 |
| 1359 | CD | GLU | A | 510 | 29.997 | 90.675 | 14.911 | 1.00 | 58.86 |
| 1360 | OE1 | GLU | A | 510 | 30.811 | 90.364 | 14.013 | 1.00 | 61.22 |
| 1361 | OE2 | GLU | A | 510 | 30.349 | 91.207 | 15.989 | 1.00 | 61.34 |
| 1362 | N | VAL | A | 511 | 25.406 | 92.479 | 11.852 | 1.00 | 36.50 |
| 1363 | CA | VAL | A | 511 | 24.935 | 93.069 | 10.606 | 1.00 | 38.05 |
| 1364 | C | VAL | A | 511 | 25.370 | 94.516 | 10.408 | 1.00 | 42.41 |
| 1365 | O | VAL | A | 511 | 25.744 | 95.205 | 11.368 | 1.00 | 41.92 |
| 1366 | CB | VAL | A | 511 | 23.403 | 93.012 | 10.508 | 1.00 | 31.84 |
| 1367 | CG1 | VAL | A | 511 | 22.927 | 91.585 | 10.669 | 1.00 | 32.42 |
| 1368 | CG2 | VAL | A | 511 | 22.787 | 93.913 | 11.553 | 1.00 | 26.87 |
| 1369 | N | THR | A | 512 | 25.310 | 94.967 | 9.154 | 1.00 | 43.58 |
| 1370 | CA | TNR | A | 512 | 25.697 | 96.331 | 8.808 | 1.00 | 48.37 |
| 1371 | C | THR | A | 512 | 24.602 | 97.306 | 9.201 | 1.00 | 50.53 |
| 1372 | O | THR | A | 512 | 23.487 | 96.894 | 9.516 | 1.00 | 51.77 |
| 1373 | CB | THR | A | 512 | 25.956 | 96.475 | 7.303 | 1.00 | 48.57 |
| 1374 | OG1 | THR | A | 512 | 24.766 | 96.140 | 6.581 | 1.00 | 46.96 |
| 1375 | CG2 | THR | A | 512 | 27.085 | 95.554 | 6.866 | 1.00 | 45.82 |
| 1376 | N | ARG | A | 513 | 24.922 | 98.598 | 9.188 | 1.00 | 53.29 |
| 1377 | CA | ARG | A | 513 | 23.952 | 99.629 | 9.544 | 1.00 | 55.54 |
| 1378 | C | ARG | A | 513 | 22.781 | 99.557 | 8.577 | 1.00 | 55.38 |
| 1379 | O | ARG | A | 513 | 21.623 | 99.741 | 8.962 | 1.00 | 55.22 |
| 1380 | CB | ARG | A | 513 | 24.594 | 101.018 | 9.464 | 1.00 | 59.23 |
| 1381 | CG | ARG | A | 513 | 23.999 | 102.037 | 10.428 | 1.00 | 65.98 |
| 1382 | CD | ARG | A | 513 | 22.519 | 102.274 | 10.184 | 1.00 | 74.12 |
| 1383 | NE | ARG | A | 513 | 21.858 | 102.827 | 11.365 | 1.00 | 80.97 |
| 1384 | CZ | ARG | A | 513 | 20.581 | 103.197 | 11.413 | 1.00 | 83.64 |
| 1385 | NH1 | ARG | A | 513 | 19.809 | 103.084 | 10.340 | 1.00 | 86.02 |
| 1386 | NH2 | ARG | A | 513 | 20.072 | 103.673 | 12.543 | 1.00 | 85.44 |
| 1387 | N | ALA | A | 514 | 23.099 | 99.285 | 7.317 | 1.00 | 55.03 |
| 1388 | CA | ALA | A | 514 | 22.096 | 99.186 | 6.268 | 1.00 | 55.46 |
| 1389 | C | ALA | A | 514 | 21.061 | 98.104 | 6.571 | 1.00 | 56.10 |
| 1390 | O | ALA | A | 514 | 19.866 | 98.390 | 6.674 | 1.00 | 56.61 |
| 1391 | CB | ALA | A | 514 | 22.775 | 98.908 | 4.927 | 1.00 | 54.05 |
| 1392 | N | GLU | A | 515 | 21.509 | 96.859 | 6.712 | 1.00 | 55.46 |
| 1393 | CA | GLU | A | 515 | 20.575 | 95.781 | 6.995 | 1.00 | 55.34 |
| 1394 | C | GLU | A | 515 | 19.949 | 95.951 | 8.371 | 1.00 | 54.83 |
| 1395 | O | GLU | A | 515 | 18.877 | 95.410 | 8.644 | 1.00 | 52.33 |
| 1396 | CB | GLU | A | 515 | 21.265 | 94.413 | 6.868 | 1.00 | 57.76 |
| 1397 | CG | GLU | A | 515 | 22.716 | 94.379 | 7.312 | 1.00 | 62.58 |
| 1398 | CD | GLU | A | 515 | 23.416 | 93.082 | 6.937 | 1.00 | 63.27 |
| 1399 | OE1 | GLU | A | 515 | 23.168 | 92.565 | 5.829 | 1.00 | 64.89 |
| 1400 | OE2 | GLU | A | 515 | 24.228 | 92.586 | 7.741 | 1.00 | 66.59 |
| 1401 | N | THR | A | 516 | 20.608 | 96.716 | 9.234 | 1.00 | 55.79 |
| 1402 | CA | THR | A | 516 | 20.072 | 96.956 | 10.566 | 1.00 | 57.81 |
| 1403 | C | THR | A | 516 | 18.818 | 97.822 | 10.464 | 1.00 | 58.51 |
| 1404 | O | THR | A | 516 | 17.988 | 97.822 | 11.374 | 1.00 | 57.71 |
| 1405 | CB | THR | A | 516 | 21.101 | 97.649 | 11.466 | 1.00 | 61.48 |
| 1406 | CG | THR | A | 516 | 20.738 | 97.544 | 12.913 | 1.00 | 66.54 |
| 1407 | CD1 | THR | A | 516 | 20.823 | 96.428 | 13.697 | 1.00 | 67.26 |
| 1408 | CD2 | THR | A | 516 | 20.140 | 98.561 | 13.725 | 1.00 | 69.56 |
| 1409 | NE1 | THR | A | 516 | 20.309 | 96.685 | 14.946 | 1.00 | 71.76 |
| 1410 | CE2 | THR | A | 516 | 19.882 | 97.987 | 14.990 | 1.00 | 70.88 |
| 1411 | CE3 | THR | A | 516 | 19.793 | 99.900 | 13.507 | 1.00 | 70.94 |
| 1412 | CZ2 | THR | A | 516 | 19.292 | 98.706 | 16.034 | 1.00 | 73.50 |
| 1413 | CZ3 | THR | A | 516 | 19.204 | 100.618 | 14.548 | 1.00 | 74.37 |
| 1414 | CH2 | THR | A | 516 | 18.961 | 100.016 | 15.796 | 1.00 | 74.71 |
| 1415 | N | GLU | A | 517 | 18.682 | 98.553 | 9.356 | 1.00 | 59.66 |
| 1416 | CA | GLU | A | 517 | 17.515 | 99.409 | 9.128 | 1.00 | 61.72 |
| 1417 | C | GLU | A | 517 | 16.301 | 98.565 | 8.746 | 1.00 | 59.87 |
| 1418 | O | GLU | A | 517 | 15.159 | 98.994 | 8.904 | 1.00 | 58.60 |
| 1419 | CB | GLU | A | 517 | 17.782 | 100.418 | 8.003 | 1.00 | 68.82 |
| 1420 | CG | GLU | A | 517 | 18.988 | 101.316 | 8.221 | 1.00 | 80.60 |
| 1421 | CD | GLU | A | 517 | 19.148 | 102.358 | 7.124 | 1.00 | 87.42 |
| 1422 | OE1 | GLU | A | 517 | 18.472 | 102.231 | 6.079 | 1.00 | 94.80 |
| 1423 | OE2 | GLU | A | 517 | 19.955 | 103.299 | 7.300 | 1.00 | 95.35 |
| 1424 | N | GLN | A | 518 | 16.555 | 97.366 | 8.233 | 1.00 | 57.92 |
| 1425 | CA | GLN | A | 518 | 15.480 | 96.468 | 7.836 | 1.00 | 55.74 |
| 1426 | C | GLN | A | 518 | 15.543 | 95.184 | 8.664 | 1.00 | 54.32 |
| 1427 | O | GLN | A | 518 | 15.207 | 94.102 | 8.176 | 1.00 | 52.85 |
| 1428 | CB | GLN | A | 518 | 15.600 | 96.146 | 6.355 | 1.00 | 54.10 |
| 1429 | N | LYS | A | 519 | 15.954 | 95.313 | 9.924 | 1.00 | 52.69 |
| 1430 | CA | LYS | A | 519 | 16.082 | 94.152 | 10.791 | 1.00 | 52.35 |
| 1431 | C | LYS | A | 519 | 14.781 | 93.394 | 10.941 | 1.00 | 51.69 |
| 1432 | O | LYS | A | 519 | 14.778 | 92.203 | 11.246 | 1.00 | 49.61 |
| 1433 | CB | LYS | A | 519 | 16.617 | 94.561 | 12.163 | 1.00 | 54.31 |
| 1434 | CG | LYS | A | 519 | 15.688 | 95.395 | 13.012 | 1.00 | 55.80 |
| 1435 | CD | LYS | A | 519 | 16.438 | 95.877 | 14.241 | 1.00 | 60.32 |
| 1436 | CE | LYS | A | 519 | 15.598 | 96.806 | 15.089 | 1.00 | 64.05 |
| 1437 | NZ | LYS | A | 519 | 16.409 | 97.377 | 16.202 | 1.00 | 70.92 |
| 1438 | N | ASP | A | 520 | 13.676 | 94.089 | 10.712 | 1.00 | 52.66 |
| 1439 | CA | ASP | A | 520 | 12.349 | 93.495 | 10.812 | 1.00 | 54.22 |
| 1440 | C | ASP | A | 520 | 12.204 | 92.391 | 9.764 | 1.00 | 53.07 |
| 1441 | O | ASP | A | 520 | 11.336 | 91.525 | 9.871 | 1.00 | 52.75 |
| 1442 | CB | ASP | A | 520 | 11.289 | 94.568 | 10.563 | 1.00 | 61.14 |
| 1443 | CG | ASP | A | 520 | 11.821 | 95.980 | 10.792 | 1.00 | 71.19 |
| 1444 | OD1 | ASP | A | 520 | 12.193 | 96.300 | 11.945 | 1.00 | 76.53 |
| 1445 | OD2 | ASP | A | 520 | 11.871 | 96.766 | 9.817 | 1.00 | 72.86 |
| 1446 | N | GLU | A | 521 | 13.067 | 92.423 | 8.757 | 1.00 | 51.01 |
| 1447 | CA | GLU | A | 521 | 13.018 | 91.443 | 7.684 | 1.00 | 50.49 |
| 1448 | C | GLU | A | 521 | 13.886 | 90.206 | 7.899 | 1.00 | 47.73 |
| 1449 | O | GLU | A | 521 | 13.762 | 89.228 | 7.165 | 1.00 | 48.58 |
| 1450 | CB | GLU | A | 521 | 13.400 | 92.107 | 6.362 | 1.00 | 53.54 |
| 1451 | CG | GLU | A | 521 | 12.423 | 93.173 | 5.901 | 1.00 | 60.11 |
| 1452 | CD | GLU | A | 521 | 12.901 | 93.895 | 4.658 | 1.00 | 65.41 |
| 1453 | OE1 | GLU | A | 521 | 13.103 | 93.239 | 3.614 | 1.00 | 67.98 |
| 1454 | OE2 | GLU | A | 521 | 13.079 | 95.129 | 4.726 | 1.00 | 73.58 |
| 1455 | N | PHE | A | 522 | 14.772 | 90.241 | 8.887 | 1.00 | 42.66 |
| 1456 | CA | PHE | A | 522 | 15.615 | 89.081 | 9.137 | 1.00 | 38.90 |
| 1457 | C | PHE | A | 522 | 14.847 | 88.017 | 9.892 | 1.00 | 35.93 |
| 1458 | O | PHE | A | 522 | 14.021 | 88.324 | 10.755 | 1.00 | 38.85 |
| 1459 | CB | PHE | A | 522 | 16.884 | 89.478 | 9.886 | 1.00 | 36.56 |
| 1460 | CG | PHE | A | 522 | 17.890 | 90.181 | 9.019 | 1.00 | 39.04 |
| 1461 | CD1 | PHE | A | 522 | 17.673 | 91.494 | 8.605 | 1.00 | 42.28 |
| 1462 | CD2 | PHE | A | 522 | 19.026 | 89.513 | 8.561 | 1.00 | 36.78 |
| 1463 | CE1 | PHE | A | 522 | 18.571 | 92.132 | 7.738 | 1.00 | 44.30 |
| 1464 | CE2 | PHE | A | 522 | 19.927 | 90.139 | 7.695 | 1.00 | 40.92 |
| 1465 | CZ | PHE | A | 522 | 19.699 | 91.454 | 7.283 | 1.00 | 39.51 |
| 1466 | N | ILE | A | 523 | 15.108 | 86.763 | 9.545 | 1.00 | 31.27 |
| 1467 | CA | ILE | A | 523 | 14.427 | 85.641 | 10.156 | 1.00 | 29.65 |
| 1468 | C | ILE | A | 523 | 15.389 | 84.551 | 10.617 | 1.00 | 29.13 |
| 1469 | O | ILE | A | 523 | 16.364 | 84.240 | 9.936 | 1.00 | 27.36 |
| 1470 | CB | ILE | A | 523 | 13.420 | 85.033 | 9.163 | 1.00 | 34.21 |
| 1471 | CG1 | ILE | A | 523 | 12.349 | 86.072 | 8.818 | 1.00 | 37.38 |
| 1472 | CG2 | ILE | A | 523 | 12.804 | 83.773 | 9.736 | 1.00 | 34.94 |
| 1473 | CD1 | ILE | A | 523 | 11.373 | 85.619 | 7.746 | 1.00 | 38.56 |
| 1474 | N | CYS | A | 524 | 15.102 | 83.990 | 11.789 | 1.00 | 26.34 |
| 1475 | CA | CYS | A | 524 | 15.901 | 82.914 | 12.361 | 1.00 | 27.10 |
| 1476 | C | CYS | A | 524 | 15.100 | 81.654 | 12.138 | 1.00 | 26.67 |
| 1477 | O | CYS | A | 524 | 13.945 | 81.590 | 12.531 | 1.00 | 27.58 |
| 1478 | CB | CYS | A | 524 | 16.103 | 83.127 | 13.874 | 1.00 | 24.27 |
| 1479 | SG | CYS | A | 524 | 16.974 | 81.777 | 14.737 | 1.00 | 37.60 |
| 1480 | N | ARG | A | 525 | 15.714 | 80.649 | 11.528 | 1.00 | 26.37 |
| 1481 | CA | ARG | A | 525 | 15.024 | 79.406 | 11.244 | 1.00 | 26.35 |
| 1482 | C | ARG | A | 525 | 15.713 | 78.181 | 11.796 | 1.00 | 26.72 |
| 1483 | O | ARG | A | 525 | 16.929 | 78.039 | 11.700 | 1.00 | 29.04 |
| 1484 | CB | ARG | A | 525 | 14.852 | 79.224 | 9.739 | 1.00 | 29.81 |
| 1485 | CG | ARG | A | 525 | 14.207 | 77.887 | 9.372 | 1.00 | 35.40 |
| 1486 | CD | ARG | A | 525 | 13.542 | 77.948 | 8.001 | 1.00 | 46.51 |
| 1487 | NE | ARG | A | 525 | 14.487 | 77.804 | 6.907 | 1.00 | 50.77 |
| 1488 | CZ | ARG | A | 525 | 14.221 | 78.139 | 5.649 | 1.00 | 55.09 |
| 1489 | NH1 | ARG | A | 525 | 13.035 | 78.649 | 5.337 | 1.00 | 54.58 |
| 1490 | NH2 | ARG | A | 525 | 15.136 | 77.950 | 4.703 | 1.00 | 56.77 |
| 1491 | N | ALA | A | 526 | 14.920 | 77.289 | 12.369 | 1.00 | 25.38 |
| 1492 | CA | ALA | A | 526 | 15.458 | 76.072 | 12.920 | 1.00 | 25.38 |
| 1493 | C | ALA | A | 526 | 14.872 | 74.879 | 12.175 | 1.00 | 25.99 |
| 1494 | O | ALA | A | 526 | 13.667 | 74.849 | 11.890 | 1.00 | 25.50 |
| 1495 | CB | ALA | A | 526 | 15.116 | 75.971 | 14.404 | 1.00 | 25.98 |
| 1496 | N | VAL | A | 527 | 15.728 | 73.916 | 11.841 | 1.00 | 23.84 |
| 1497 | CA | VAL | A | 527 | 15.267 | 72.705 | 11.198 | 1.00 | 25.15 |
| 1498 | C | VAL | A | 527 | 15.522 | 71.628 | 12.220 | 1.00 | 26.30 |
| 1499 | O | VAL | A | 527 | 16.642 | 71.481 | 12.706 | 1.00 | 26.20 |
| 1500 | CB | VAL | A | 527 | 16.043 | 72.365 | 9.923 | 1.00 | 24.43 |
| 1501 | CG1 | VAL | A | 527 | 15.610 | 71.012 | 9.439 | 1.00 | 26.95 |
| 1502 | CG2 | VAL | A | 527 | 15.781 | 73.409 | 8.849 | 1.00 | 28.13 |
| 1503 | N | HIS | A | 528 | 14.485 | 70.862 | 12.531 | 1.00 | 26.24 |
| 1504 | CA | HIS | A | 528 | 14.582 | 69.828 | 13.536 | 1.00 | 29.14 |
| 1505 | C | HIS | A | 528 | 13.498 | 68.778 | 13.295 | 1.00 | 31.86 |
| 1506 | O | HIS | A | 528 | 12.384 | 69.091 | 12.861 | 1.00 | 31.05 |
| 1507 | CB | HIS | A | 528 | 14.428 | 70.479 | 14.921 | 1.00 | 29.03 |
| 1508 | CG | HIS | A | 528 | 14.518 | 69.523 | 16.065 | 1.00 | 29.34 |
| 1509 | ND1 | HIS | A | 528 | 13.491 | 68.666 | 16.404 | 1.00 | 33.33 |
| 1510 | CD2 | HIS | A | 528 | 15.510 | 69.283 | 16.956 | 1.00 | 28.93 |
| 1511 | CE1 | HIS | A | 528 | 13.845 | 67.945 | 17.449 | 1.00 | 26.07 |
| 1512 | NE2 | HIS | A | 528 | 15.070 | 68.298 | 17.805 | 1.00 | 32.19 |
| 1513 | N | CLU | A | 529 | 13.843 | 67.536 | 13.599 | 1.00 | 34.08 |
| 1514 | CA | CLU | A | 529 | 12.964 | 66.393 | 13.424 | 1.00 | 39.76 |
| 1515 | C | CLU | A | 529 | 11.563 | 66.490 | 14.034 | 1.00 | 41.50 |
| 1516 | O | CLU | A | 529 | 10.580 | 66.190 | 13.360 | 1.00 | 41.48 |
| 1517 | CB | GLU | A | 529 | 13.641 | 65.138 | 13.978 | 1.00 | 43.03 |
| 1518 | CG | GLU | A | 529 | 12.702 | 63.935 | 14.014 | 1.00 | 56.19 |
| 1519 | CD | GLU | A | 529 | 13.239 | 62.773 | 14.826 | 1.00 | 62.66 |
| 1520 | OE1 | GLU | A | 529 | 12.489 | 61.788 | 15.007 | 1.00 | 63.52 |
| 1521 | OE2 | GLU | A | 529 | 14.402 | 62.842 | 15.281 | 1.00 | 65.43 |
| 1522 | N | ALA | A | 530 | 11.479 | 66.897 | 15.300 | 1.00 | 42.51 |
| 1523 | CA | ALA | A | 530 | 10.201 | 66.978 | 16.006 | 1.00 | 46.03 |
| 1524 | C | ALA | A | 530 | 9.269 | 68.105 | 15.596 | 1.00 | 49.29 |
| 1525 | O | ALA | A | 530 | 8.124 | 68.153 | 16.044 | 1.00 | 50.87 |
| 1526 | CB | ALA | A | 530 | 10.443 | 67.043 | 17.513 | 1.00 | 39.71 |
| 1527 | N | ALA | A | 531 | 9.751 | 69.005 | 14.748 | 1.00 | 52.89 |
| 1528 | CA | ALA | A | 531 | 8.949 | 70.133 | 14.302 | 1.00 | 56.14 |
| 1529 | C | ALA | A | 531 | 8.030 | 69.799 | 13.124 | 1.00 | 59.66 |
| 1530 | O | ALA | A | 531 | 8.258 | 70.243 | 12.004 | 1.00 | 61.00 |
| 1531 | CB | ALA | A | 531 | 9.864 | 71.295 | 13.938 | 1.00 | 52.83 |
| 1532 | N | SER | A | 532 | 6.992 | 69.012 | 13.370 | 1.00 | 63.07 |
| 1533 | CA | SER | A | 532 | 6.056 | 68.673 | 12.305 | 1.00 | 66.03 |
| 1534 | C | SER | A | 532 | 5.006 | 69.778 | 12.250 | 1.00 | 66.08 |
| 1535 | O | SER | A | 532 | 4.569 | 70.283 | 13.286 | 1.00 | 65.35 |
| 1536 | CB | SER | A | 532 | 5.384 | 67.330 | 12.593 | 1.00 | 68.98 |
| 1537 | OG | SER | A | 532 | 4.526 | 67.416 | 13.716 | 1.00 | 74.64 |
| 1538 | N | PRO | A | 533 | 4.581 | 70.173 | 11.043 | 1.00 | 66.51 |
| 1539 | CA | PRO | A | 533 | 4.931 | 69.713 | 9.696 | 1.00 | 65.99 |
| 1540 | C | PRO | A | 533 | 6.251 | 70.226 | 9.117 | 1.00 | 63.48 |
| 1541 | O | PRO | A | 533 | 6.760 | 71.273 | 9.518 | 1.00 | 64.33 |
| 1542 | CB | PRO | A | 533 | 3.757 | 70.203 | 8.873 | 1.00 | 69.06 |
| 1543 | CG | PRO | A | 533 | 3.539 | 71.556 | 9.471 | 1.00 | 71.08 |
| 1544 | CB | PRO | A | 533 | 3.570 | 71.242 | 10.963 | 1.00 | 70.23 |
| 1545 | N | SER | A | 534 | 6.783 | 69.466 | 8.165 | 1.00 | 59.21 |
| 1546 | CA | SER | A | 534 | 8.009 | 69.804 | 7.452 | 1.00 | 55.01 |
| 1547 | C | SER | A | 534 | 9.274 | 70.148 | 8.248 | 1.00 | 50.27 |
| 1548 | O | SER | A | 534 | 10.130 | 70.875 | 7.753 | 1.00 | 48.58 |
| 1549 | CB | SER | A | 534 | 7.706 | 70.934 | 6.475 | 1.00 | 58.20 |
| 1550 | OG | SER | A | 534 | 7.074 | 72.013 | 7.138 | 1.00 | 63.36 |
| 1551 | N | GLN | A | 535 | 9.387 | 69.616 | 9.461 | 1.00 | 44.89 |
| 1552 | CA | GLN | A | 535 | 10.543 | 69.829 | 10.330 | 1.00 | 42.88 |
| 1553 | C | GLN | A | 535 | 11.194 | 71.205 | 10.277 | 1.00 | 41.55 |
| 1554 | O | GLN | A | 535 | 12.419 | 71.321 | 10.294 | 1.00 | 40.80 |
| 1555 | CB | GLN | A | 535 | 11.633 | 68.769 | 10.080 | 1.00 | 37.08 |
| 1556 | CG | GLN | A | 535 | 11.302 | 67.731 | 9.056 | 1.00 | 47.40 |
| 1557 | CD | GLN | A | 535 | 10.101 | 66.916 | 9.431 | 1.00 | 44.36 |
| 1558 | OE1 | GLN | A | 535 | 9.193 | 66.738 | 8.625 | 1.00 | 47.59 |
| 1559 | NE2 | GLN | A | 535 | 10.079 | 66.415 | 10.664 | 1.00 | 54.70 |
| 1560 | N | THR | A | 536 | 10.387 | 72.252 | 10.226 | 1.00 | 39.97 |
| 1561 | CA | THR | A | 536 | 10.950 | 73.588 | 10.207 | 1.00 | 40.62 |
| 1562 | C | THR | A | 536 | 10.113 | 74.575 | 11.023 | 1.00 | 37.57 |
| 1563 | O | THR | A | 536 | 8.893 | 74.558 | 10.975 | 1.00 | 40.85 |
| 1564 | CB | THR | A | 536 | 11.137 | 74.092 | 8.747 | 1.00 | 42.38 |
| 1565 | OG1 | THR | A | 536 | 11.458 | 75.488 | 8.763 | 1.00 | 44.59 |
| 1566 | CG2 | THR | A | 536 | 9.881 | 73.870 | 7.932 | 1.00 | 49.65 |
| 1567 | N | VAL | A | 537 | 10.788 | 75.410 | 11.801 | 1.00 | 35.29 |
| 1568 | CA | VAL | A | 537 | 10.147 | 76.418 | 12.632 | 1.00 | 31.25 |
| 1569 | C | VAL | A | 537 | 10.996 | 77.678 | 12.537 | 1.00 | 29.01 |
| 1570 | O | VAL | A | 537 | 12.218 | 77.615 | 12.699 | 1.00 | 30.11 |
| 1571 | CB | VAL | A | 537 | 10.106 | 75.996 | 14.117 | 1.00 | 34.80 |
| 1572 | CG1 | VAL | A | 537 | 9.452 | 77.084 | 14.939 | 1.00 | 35.52 |
| 1573 | CG2 | VAL | A | 537 | 9.341 | 74.712 | 14.278 | 1.00 | 37.19 |
| 1574 | N | GLN | A | 538 | 10.365 | 78.820 | 12.292 | 1.00 | 26.73 |
| 1575 | CA | GLN | A | 538 | 11.107 | 80.067 | 12.167 | 1.00 | 26.74 |
| 1576 | C | GLN | A | 538 | 10.376 | 81.271 | 12.732 | 1.00 | 27.53 |
| 1577 | O | GLN | A | 538 | 9.169 | 81.249 | 12.902 | 1.00 | 29.89 |
| 1578 | CB | GLN | A | 538 | 11.424 | 80.335 | 10.700 | 1.00 | 28.48 |
| 1579 | CG | GLN | A | 538 | 10.208 | 80.624 | 9.834 | 1.00 | 25.88 |
| 1580 | CD | CLN | A | 538 | 10.595 | 80.853 | 8.378 | 1.00 | 28.51 |
| 1581 | OE1 | GLN | A | 538 | 11.390 | 80.102 | 7.810 | 1.00 | 31.66 |
| 1582 | NE2 | GLN | A | 538 | 10.035 | 81.885 | 7.772 | 1.00 | 27.39 |
| 1583 | N | ARG | A | 539 | 11.113 | 82.334 | 13.013 | 1.00 | 28.05 |
| 1584 | CA | ARG | A | 539 | 10.493 | 83.530 | 13.535 | 1.00 | 30.72 |
| 1585 | C | ARG | A | 539 | 11.288 | 84.742 | 13.114 | 1.00 | 31.33 |
| 1586 | O | ARG | A | 539 | 12.517 | 84.703 | 13.077 | 1.00 | 32.42 |
| 1587 | CB | ARG | A | 539 | 10.420 | 83.463 | 15.061 | 1.00 | 36.30 |
| 1588 | CG | ARG | A | 539 | 9.488 | 84.476 | 15.686 | 1.00 | 45.60 |
| 1589 | CB | ARG | A | 539 | 8.741 | 83.829 | 16.830 | 1.00 | 57.97 |
| 1590 | NE | ARG | A | 539 | 8.118 | 82.591 | 16.371 | 1.00 | 69.46 |
| 1591 | CZ | ARG | A | 539 | 7.453 | 81.745 | 17.150 | 1.00 | 78.18 |
| 1592 | NH1 | ARG | A | 539 | 7.316 | 81.996 | 18.447 | 1.00 | 83.01 |
| 1593 | NH2 | ARG | A | 539 | 6.927 | 80.641 | 16.630 | 1.00 | 81.89 |
| 1594 | N | ALA | A | 540 | 10.578 | 85.818 | 12.799 | 1.00 | 31.92 |
| 1595 | CA | ALA | A | 540 | 11.207 | 87.054 | 12.383 | 1.00 | 34.50 |
| 1596 | C | ALA | A | 540 | 11.691 | 87.774 | 13.634 | 1.00 | 36.68 |
| 1597 | O | ALA | A | 540 | 11.190 | 87.532 | 14.739 | 1.00 | 34.62 |
| 1598 | CB | ALA | A | 540 | 10.209 | 87.907 | 11.621 | 1.00 | 33.91 |
| 1599 | N | VAL | A | 541 | 12.694 | 88.626 | 13.460 | 1.00 | 39.73 |
| 1600 | CA | VAL | A | 541 | 13.254 | 89.386 | 14.572 | 1.00 | 44.47 |
| 1601 | C | VAL | A | 541 | 12.164 | 90.249 | 15.218 | 1.00 | 46.91 |
| 1602 | O | VAL | A | 541 | 11.595 | 91.124 | 14.573 | 1.00 | 44.53 |
| 1603 | CB | VAL | A | 541 | 14.415 | 90.295 | 14.090 | 1.00 | 42.49 |
| 1604 | CG1 | VAL | A | 541 | 14.715 | 91.358 | 15.130 | 1.00 | 47.96 |
| 1605 | CG2 | VAL | A | 541 | 15.655 | 89.460 | 13.831 | 1.00 | 42.43 |
| 1606 | N | SER | A | 542 | 11.869 | 89.997 | 16.489 | 1.00 | 52.25 |
| 1607 | CA | SER | A | 542 | 10.840 | 90.771 | 17.174 | 1.00 | 60.05 |
| 1608 | C | SER | A | 542 | 11.374 | 92.174 | 17.421 | 1.00 | 64.25 |
| 1609 | O | SER | A | 542 | 12.550 | 92.353 | 17.743 | 1.00 | 65.22 |
| 1610 | CB | SER | A | 542 | 10.443 | 90.110 | 18.500 | 1.00 | 60.43 |
| 1611 | OG | SER | A | 542 | 11.530 | 90.076 | 19.404 | 1.00 | 65.30 |
| 1612 | N | VAL | A | 543 | 10.510 | 93.168 | 17.256 | 1.00 | 68.19 |
| 1613 | CA | VAL | A | 543 | 10.906 | 94.557 | 17.444 | 1.00 | 72.45 |
| 1614 | C | VAL | A | 543 | 9.828 | 95.323 | 18.202 | 1.00 | 74.32 |
| 1615 | O | VAL | A | 543 | 8.970 | 95.979 | 17.603 | 1.00 | 75.99 |
| 1616 | CB | VAL | A | 543 | 11.148 | 95.247 | 16.084 | 1.00 | 74.41 |
| 1617 | CG1 | VAL | A | 543 | 11.530 | 96.705 | 16.299 | 1.00 | 76.37 |
| 1618 | CG2 | VAL | A | 543 | 12.233 | 94.514 | 15.318 | 1.00 | 75.78 |
| 1619 | O | HOH | A | 1 | 24.509 | 77.358 | 12.191 | 1.00 | 54.75 |
| 1620 | O | HOH | A | 2 | 7.537 | 85.444 | 12.546 | 1.00 | 37.27 |
| 1621 | O | HOH | A | 3 | 26.419 | 96.436 | 20.705 | 1.00 | 46.90 |
| 1622 | O | HOH | A | 4 | 27.452 | 65.238 | 9.563 | 1.00 | 44.93 |
| 1623 | O | HOH | A | 5 | 8.585 | 79.087 | 17.993 | 1.00 | 46.77 |
| 1624 | O | HOH | A | 6 | 22.224 | 80.929 | 7.089 | 1.00 | 40.32 |
| 1625 | O | HOH | A | 7 | 17.272 | 54.803 | 2.090 | 1.00 | 50.21 |
| 1626 | O | HOH | A | 8 | 30.340 | 75.004 | 30.685 | 1.00 | 61.75 |
| 1627 | O | HOH | A | 9 | 24.537 | 65.977 | 6.710 | 1.00 | 39.17 |
| 1628 | O | HOH | A | 10 | 16.841 | 94.781 | 2.897 | 1.00 | 64.36 |
| 1629 | O | HOH | A | 11 | 22.950 | 61.704 | 0.621 | 1.00 | 45.74 |
| 1630 | O | HOH | A | 12 | 28.469 | 65.195 | 7.094 | 1.00 | 39.34 |
| 1631 | O | HOH | A | 13 | 53.771 | 76.970 | 5.717 | 1.00 | 56.15 |
| 1632 | O | HOH | A | 14 | 28.128 | 87.403 | 13.055 | 1.00 | 46.48 |
| 1633 | O | HOH | A | 15 | 23.871 | 76.354 | 23.810 | 1.00 | 36.55 |
| 1634 | O | HOH | A | 16 | 28.656 | 78.595 | 14.195 | 1.00 | 48.95 |
| 1635 | O | HOH | A | 17 | 26.439 | 59.235 | 16.698 | 1.00 | 50.90 |
| 1636 | O | HOH | A | 18 | 48.822 | 76.573 | 10.696 | 1.00 | 58.83 |
| 1637 | O | HOH | A | 19 | 46.016 | 72.983 | 15.254 | 1.00 | 61.24 |
| 1638 | O | HOH | A | 20 | 18.011 | 59.153 | 0.608 | 1.00 | 58.37 |
| 1639 | O | HOH | A | 21 | 10.586 | 67.712 | 6.227 | 1.00 | 57.48 |
| 1640 | O | HOH | A | 22 | 39.217 | 50.176 | 20.140 | 1.00 | 60.62 |
| 1641 | O | HOH | A | 23 | 19.990 | 55.647 | 11.936 | 1.00 | 49.14 |
| 1642 | O | HOH | A | 24 | 18.092 | 104.938 | 6.774 | 1.00 | 63.67 |
| 1643 | O | HOH | A | 25 | 11.413 | 86.589 | 17.509 | 1.00 | 40.50 |
| 1644 | O | HOH | A | 26 | 55.940 | 62.358 | 0.863 | 1.00 | 58.12 |
| 1645 | O | HOH | A | 27 | 26.856 | 64.971 | 15.189 | 1.00 | 52.34 |
| 1646 | O | HOH | A | 28 | 32.914 | 64.797 | 12.453 | 1.00 | 52.41 |
| 1647 | O | HOH | A | 29 | 23.938 | 107.034 | 22.326 | 1.00 | 63.47 |
| 1648 | O | HOH | A | 30 | 28.142 | 76.042 | 14.972 | 1.00 | 56.91 |
| 1649 | O | HOH | A | 31 | 34.884 | 65.010 | 3.193 | 1.00 | 49.06 |
| 1650 | O | HOH | A | 32 | 10.166 | 82.530 | 4.732 | 1.00 | 59.31 |
| 1651 | O | HOH | A | 33 | 28.781 | 47.212 | 5.405 | 1.00 | 51.40 |
| 1652 | O | HOH | A | 34 | 26.373 | 54.646 | 2.184 | 1.00 | 49.71 |
| 1653 | O | HOH | A | 35 | 50.871 | 71.532 | 11.654 | 1.00 | 64.98 |
| 1654 | O | HOH | A | 36 | 14.970 | 75.672 | 2.416 | 1.00 | 55.97 |
| 1655 | O | HOH | A | 37 | 7.183 | 75.882 | 18.351 | 1.00 | 56.10 |
| 1656 | O | HOH | A | 38 | 8.889 | 71.461 | 23.144 | 1.00 | 48.69 |
| 1657 | O | HOH | A | 39 | 16.590 | 67.594 | 29.822 | 1.00 | 58.57 |
| 1658 | O | HOH | A | 40 | 38.458 | 52.685 | 18.905 | 1.00 | 54.81 |
| 1659 | O | HOH | A | 41 | 54.406 | 64.045 | 8.519 | 1.00 | 65.02 |
| 1660 | O | HOH | A | 42 | 10.751 | 53.102 | 5.496 | 1.00 | 60.95 |
| 1661 | O | HOH | A | 43 | 52.001 | 82.188 | 9.439 | 1.00 | 59.29 |
| 1662 | O | HOH | A | 44 | 16.985 | 77.359 | 2.354 | 1.00 | 64.70 |
| 1663 | O | HOH | A | 45 | 20.295 | 50.729 | 1.374 | 1.00 | 57.91 |
| 1664 | O | HOH | A | 46 | 23.952 | 95.782 | 3.814 | 1.00 | 54.20 |
| 1665 | O | HOH | A | 47 | 14.130 | 79.241 | 2.012 | 1.00 | 59.40 |
| 1666 | O | HOH | A | 48 | 42.373 | 77.528 | 8.231 | 1.00 | 61.07 |
| 1667 | O | HOH | A | 49 | 49.811 | 60.323 | 14.343 | 1.00 | 62.77 |
| 1668 | O | HOH | A | 50 | 32.587 | 60.842 | 17.597 | 1.00 | 54.90 |
| 1669 | O | HOH | A | 51 | 26.700 | 67.334 | 3.009 | 1.00 | 59.03 |
| 1670 | O | HOH | A | 52 | 27.137 | 97.388 | 12.542 | 1.00 | 57.55 |
| 1671 | O | HOH | A | 53 | 19.726 | 104.219 | 18.295 | 1.00 | 55.02 |
| 1672 | O | HOH | A | 54 | 27.571 | 78.794 | 11.807 | 1.00 | 62.62 |
| 1673 | O | HOH | A | 55 | 10.438 | 93.153 | 21.642 | 1.00 | 64.18 |
| 1674 | O | HOH | A | 56 | 20.189 | 101.789 | 21.921 | 1.00 | 57.80 |
| 1675 | O | HOH | A | 57 | 45.236 | 75.729 | 14.048 | 1.00 | 68.28 |
| 1676 | O | HOH | A | 58 | 28.726 | 86.130 | 15.755 | 1.00 | 42.77 |
| 1677 | O | HOH | A | 59 | 15.761 | 61.916 | 22.864 | 1.00 | 49.12 |
| 1678 | O | HOH | A | 60 | 18.673 | 97.101 | 18.412 | 1.00 | 60.00 |
| 1679 | O | HOH | A | 61 | 51.586 | 62.841 | 8.977 | 1.00 | 66.82 |
| 1680 | O | HOH | A | 62 | 12.665 | 48.318 | 9.830 | 1.00 | 61.58 |
| 1681 | O | HOH | A | 63 | 10.994 | 69.841 | 26.147 | 1.00 | 54.56 |
| 1682 | O | HOH | A | 64 | 34.004 | 50.096 | 9.090 | 1.00 | 52.20 |
| 1683 | O | HOH | A | 65 | 22.928 | 54.076 | 3.637 | 1.00 | 63.55 |
| 1684 | O | HOH | A | 66 | 13.124 | 80.438 | 22.463 | 1.00 | 49.41 |
| 1685 | O | HOH | A | 67 | 14.496 | 84.734 | 3.782 | 1.00 | 59.74 |
| 1686 | O | HOH | A | 68 | 33.203 | 84.033 | 9.037 | 1.00 | 61.59 |
| 1687 | O | HOH | A | 69 | 25.151 | 70.683 | 28.546 | 1.00 | 58.32 |
| 1688 | O | HOH | A | 70 | 43.528 | 47.541 | 7.082 | 1.00 | 67.46 |
| 1689 | O | HOH | A | 71 | 28.976 | 58.922 | 3.873 | 1.00 | 56.29 |
| 1690 | O | HOH | A | 72 | 39.085 | 57.630 | 3.442 | 1.00 | 57.89 |
| 1691 | O | HOH | A | 73 | 34.708 | 46.624 | 11.157 | 1.00 | 61.84 |
| 1692 | O | HOH | A | 74 | 3.300 | 67.675 | 10.833 | 1.00 | 70.53 |
| 1693 | O | HOH | A | 75 | 23.591 | 70.505 | 16.477 | 1.00 | 53.22 |
| 1694 | O | HOH | A | 76 | 15.993 | 56.650 | 1.105 | 1.00 | 60.73 |
| 1695 | O | HOH | A | 77 | 51.586 | 54.551 | 5.644 | 1.00 | 64.35 |
| 1696 | O | HOH | A | 78 | 14.626 | 95.986 | 18.718 | 1.00 | 59.27 |
| 1697 | O | HOH | A | 79 | 23.734 | 74.708 | 12.176 | 1.00 | 53.70 |
| 1698 | O | HOH | A | 80 | 17.100 | 96.977 | 3.795 | 1.00 | 60.32 |
| 1699 | O | HOH | A | 81 | 9.533 | 67.755 | 26.736 | 1.00 | 56.40 |
| 1700 | O | HOH | A | 82 | 18.888 | 66.846 | 28.479 | 1.00 | 62.11 |
| 1701 | O | HOH | A | 83 | 43.839 | 71.746 | 16.116 | 1.00 | 57.66 |
| 1702 | O | HOH | A | 84 | 8.517 | 74.508 | 22.311 | 1.00 | 61.70 |
| 1703 | O | HOH | A | 85 | 41.870 | 49.996 | 21.891 | 1.00 | 61.38 |
| 1704 | O | HOH | A | 86 | 14.562 | 94.919 | 2.718 | 1.00 | 67.86 |
| 1705 | O | HOH | A | 87 | 28.791 | 96.969 | 19.836 | 1.00 | 61.99 |
| 1706 | O | HOH | A | 88 | 23.437 | 78.234 | 7.551 | 1.00 | 54.45 |
| 1707 | O | HOH | A | 89 | 29.614 | 80.314 | 4.323 | 1.00 | 57.86 |
| 1708 | O | HOH | A | 90 | 28.546 | 67.893 | 5.438 | 1.00 | 58.43 |
| 1709 | O | HOH | A | 91 | 47.040 | 72.908 | 13.045 | 1.00 | 67.78 |
| 1710 | O | HOH | A | 92 | 42.407 | 51.656 | 18.123 | 1.00 | 65.22 |
| 1711 | O | HOH | A | 93 | 23.749 | 65.735 | −2.072 | 1.00 | 60.31 |
| 1712 | O | HOH | A | 94 | 44.018 | 74.292 | 16.194 | 1.00 | 61.54 |
| 1713 | O | HOH | A | 95 | 11.601 | 46.086 | 10.312 | 1.00 | 67.64 |
| 1714 | O | HOH | A | 96 | 6.175 | 66.153 | 17.508 | 1.00 | 56.59 |
| 1715 | O | HOH | A | 97 | 31.504 | 47.151 | 3.997 | 1.00 | 64.62 |
| 1716 | O | HOH | A | 98 | 42.312 | 75.926 | 6.322 | 1.00 | 66.03 |
| 1717 | O | HOH | A | 99 | 31.022 | 57.142 | 18.155 | 1.00 | 62.65 |
| 1718 | O | HOH | A | 100 | 17.995 | 104.383 | 3.752 | 1.00 | 63.95 |
| 1719 | O | HOH | A | 101 | 10.716 | 86.701 | −4.491 | 1.00 | 63.86 |
| 1720 | O | HOH | A | 102 | 27.763 | 72.631 | 16.379 | 1.00 | 62.58 |
| 1721 | O | HOH | A | 103 | 15.381 | 105.037 | 7.270 | 1.00 | 58.40 |
| 1722 | O | HOH | A | 104 | 47.651 | 78.799 | 15.518 | 1.00 | 61.34 |
| 1723 | O | HOH | A | 105 | 34.927 | 94.559 | 9.949 | 1.00 | 65.87 |
| 1724 | O | HOH | A | 106 | 9.087 | 82.761 | 20.779 | 1.00 | 61.27 |
| 1725 | O | HOH | A | 107 | 22.682 | 53.866 | 0.739 | 1.00 | 67.17 |
| 1726 | O | HOH | A | 108 | 37.349 | 58.563 | −1.263 | 1.00 | 54.90 |
| 1727 | O | HOH | A | 109 | 24.982 | 97.874 | 2.783 | 1.00 | 66.23 |
| 1728 | O | HOH | A | 110 | 31.881 | 73.012 | 28.973 | 1.00 | 59.96 |
| 1729 | O | HOH | A | 111 | 47.953 | 60.989 | 1.790 | 1.00 | 64.92 |
| 1730 | O | HOH | A | 112 | 42.129 | 73.312 | 10.101 | 1.00 | 65.68 |
| 1731 | O | HOH | A | 113 | 36.875 | 58.541 | 1.642 | 1.00 | 61.47 |
| 1732 | O | HOH | A | 114 | 35.326 | 52.952 | 2.865 | 1.00 | 60.20 |
| 1733 | O | HOH | A | 115 | 6.136 | 77.374 | 20.212 | 1.00 | 59.29 |
| 1734 | O | HOH | A | 116 | 25.741 | 94.929 | 18.879 | 1.00 | 53.05 |
| 1735 | O | HOH | A | 117 | 54.362 | 58.376 | 9.533 | 1.00 | 69.15 |
| 1736 | O | HOH | A | 118 | 23.326 | 103.523 | 17.163 | 1.00 | 62.28 |
| 1737 | O | HOH | A | 119 | 5.978 | 83.975 | 15.596 | 1.00 | 59.61 |
| 1738 | O | HOH | A | 120 | 33.072 | 54.315 | 0.721 | 1.00 | 61.94 |
| 1739 | O | HOH | A | 121 | 37.197 | 45.372 | 11.843 | 1.00 | 64.45 |
| 1740 | O | HOH | A | 122 | 11.834 | 65.339 | 3.962 | 1.00 | 63.01 |
| 1741 | O | HOH | A | 123 | 16.132 | 79.865 | 0.613 | 1.00 | 59.29 |
| 1742 | O | HOH | A | 124 | 12.804 | 74.215 | 0.796 | 1.00 | 61.69 |
| 1743 | O | HOH | A | 125 | 6.166 | 72.199 | 12.094 | 1.00 | 69.10 |
| 1744 | O | HOH | A | 126 | 46.672 | 51.916 | 3.111 | 1.00 | 61.79 |
| 1745 | O | HOH | A | 127 | 21.940 | 56.684 | −1.243 | 1.00 | 63.74 |
| 1746 | O | HOH | A | 128 | 19.803 | 72.626 | 40.522 | 1.00 | 67.04 |
| 1747 | O | HOH | A | 129 | 28.002 | 103.109 | 11.797 | 1.00 | 62.20 |
| 1748 | O | HOH | A | 130 | 16.264 | 66.529 | 27.554 | 1.00 | 66.48 |
| 1749 | O | HOH | A | 131 | 38.684 | 71.694 | 5.498 | 1.00 | 70.79 |
| 1750 | O | HOH | A | 132 | 30.875 | 98.591 | 19.410 | 1.00 | 72.41 |
| 1751 | O | HOH | A | 133 | 20.444 | 64.387 | 22.091 | 1.00 | 62.09 |
| 1752 | O | HOH | A | 134 | 16.959 | 52.193 | 1.564 | 1.00 | 61.32 |
| 1753 | O | HOH | A | 135 | 43.707 | 44.495 | 7.969 | 1.00 | 68.49 |
| 1754 | O | HOH | A | 136 | 27.297 | 51.198 | 3.047 | 1.00 | 69.04 |
| 1755 | O | HOH | A | 137 | 45.002 | 47.522 | 9.091 | 1.00 | 63.49 |
| 1756 | O | HOH | A | 138 | 20.670 | 82.001 | 0.631 | 1.00 | 53.37 |
| 1757 | O | HOH | A | 139 | 18.747 | 100.351 | 20.086 | 1.00 | 59.07 |
| 1758 | O | HOH | A | 140 | 43.001 | 46.804 | 15.782 | 1.00 | 65.35 |
| 1759 | O | HOH | A | 141 | 56.594 | 54.897 | 10.204 | 1.00 | 67.74 |
| 1760 | O | HOH | A | 142 | 16.980 | 94.578 | 0.460 | 1.00 | 67.40 |
| 1761 | O | HOH | A | 143 | 34.493 | 68.011 | 5.655 | 1.00 | 64.43 |
| 1762 | O | HOH | A | 144 | 7.561 | 88.413 | 1.394 | 1.00 | 67.27 |
| 1763 | O | HOH | A | 145 | 55.370 | 75.436 | 6.692 | 1.00 | 70.07 |
| 1764 | N | VAL | B | 336 | 42.982 | 59.443 | 29.109 | 1.00 | 58.93 |
| 1765 | CA | VAL | B | 336 | 43.788 | 60.200 | 30.107 | 1.00 | 60.44 |
| 1766 | C | VAL | B | 336 | 44.173 | 61.587 | 29.599 | 1.00 | 60.07 |
| 1767 | O | VAL | B | 336 | 45.211 | 61.753 | 28.958 | 1.00 | 61.48 |
| 1768 | CB | VAL | B | 336 | 45.079 | 59.445 | 30.453 | 1.00 | 60.81 |
| 1769 | CG1 | VAL | B | 336 | 45.794 | 60.136 | 31.600 | 1.00 | 61.71 |
| 1770 | CG2 | VAL | B | 336 | 44.755 | 58.010 | 30.798 | 1.00 | 64.73 |
| 1771 | N | SER | B | 337 | 43.343 | 62.583 | 29.891 | 1.00 | 57.95 |
| 1772 | CA | SER | B | 337 | 43.617 | 63.945 | 29.452 | 1.00 | 56.60 |
| 1773 | C | SER | B | 337 | 43.758 | 64.917 | 30.624 | 1.00 | 55.17 |
| 1774 | O | SER | B | 337 | 43.338 | 64.619 | 31.748 | 1.00 | 54.55 |
| 1775 | CB | SER | B | 337 | 42.522 | 64.415 | 28.497 | 1.00 | 57.92 |
| 1776 | OG | SER | B | 337 | 41.244 | 64.111 | 29.013 | 1.00 | 64.70 |
| 1777 | N | ALA | B | 338 | 44.362 | 66.073 | 30.357 | 1.00 | 52.40 |
| 1778 | CA | ALA | B | 338 | 44.590 | 67.078 | 31.389 | 1.00 | 50.69 |
| 1779 | C | ALA | B | 338 | 44.300 | 68.491 | 30.908 | 1.00 | 50.25 |
| 1780 | O | ALA | B | 338 | 44.538 | 68.828 | 29.746 | 1.00 | 51.17 |
| 1781 | CB | ALA | B | 338 | 46.027 | 66.989 | 31.889 | 1.00 | 50.21 |
| 1782 | N | TYR | B | 339 | 43.792 | 69.319 | 31.816 | 1.00 | 48.22 |
| 1783 | CA | TYR | B | 339 | 43.461 | 70.701 | 31.495 | 1.00 | 46.82 |
| 1784 | C | TYR | B | 339 | 43.834 | 71.610 | 32.669 | 1.00 | 44.51 |
| 1785 | O | TYR | B | 339 | 43.711 | 71.228 | 33.832 | 1.00 | 42.27 |
| 1786 | CB | TYR | B | 339 | 41.962 | 70.835 | 31.185 | 1.00 | 50.52 |
| 1787 | CG | TYR | B | 339 | 41.402 | 69.731 | 30.307 | 1.00 | 58.40 |
| 1788 | CD1 | TYR | B | 339 | 41.155 | 68.456 | 30.826 | 1.00 | 61.23 |
| 1789 | CD2 | TYR | B | 339 | 41.144 | 69.952 | 28.949 | 1.00 | 60.99 |
| 1790 | CE1 | TYR | B | 339 | 40.671 | 67.426 | 30.019 | 1.00 | 62.71 |
| 1791 | CE2 | TYR | B | 339 | 40.657 | 68.926 | 28.131 | 1.00 | 63.53 |
| 1792 | CZ | TYR | B | 339 | 40.424 | 67.667 | 28.673 | 1.00 | 64.33 |
| 1793 | OH | TYR | B | 339 | 39.956 | 66.647 | 27.870 | 1.00 | 65.07 |
| 1794 | N | LEU | B | 340 | 44.309 | 72.807 | 32.354 | 1.00 | 41.90 |
| 1795 | CA | LEU | B | 340 | 44.695 | 73.769 | 33.372 | 1.00 | 41.06 |
| 1796 | C | LEU | B | 340 | 43.929 | 75.029 | 33.022 | 1.00 | 41.10 |
| 1797 | O | LEU | B | 340 | 43.953 | 75.464 | 31.876 | 1.00 | 43.45 |
| 1798 | CB | LEU | B | 340 | 46.208 | 74.017 | 33.321 | 1.00 | 36.93 |
| 1799 | CG | LEU | B | 340 | 46.830 | 74.911 | 34.399 | 1.00 | 35.91 |
| 1800 | CD1 | LEU | B | 340 | 46.500 | 74.371 | 35.786 | 1.00 | 34.60 |
| 1801 | CD2 | LEU | B | 340 | 48.336 | 74.979 | 34.194 | 1.00 | 35.51 |
| 1802 | N | SER | B | 341 | 43.232 | 75.610 | 33.992 | 1.00 | 39.82 |
| 1803 | CA | SER | B | 341 | 42.448 | 76.810 | 33.715 | 1.00 | 39.85 |
| 1804 | C | SER | B | 341 | 42.955 | 78.002 | 34.486 | 1.00 | 37.88 |
| 1805 | O | SER | B | 341 | 43.524 | 77.863 | 35.556 | 1.00 | 42.10 |
| 1806 | CB | SER | B | 341 | 40.973 | 76.575 | 34.058 | 1.00 | 39.34 |
| 1807 | OG | SER | B | 341 | 40.808 | 76.253 | 35.428 | 1.00 | 44.58 |
| 1808 | N | ARG | B | 342 | 42.732 | 79.185 | 33.942 | 1.00 | 36.98 |
| 1809 | CA | ARG | B | 342 | 43.163 | 80.413 | 34.583 | 1.00 | 35.20 |
| 1810 | C | ARG | B | 342 | 42.183 | 80.747 | 35.707 | 1.00 | 33.18 |
| 1811 | O | ARG | B | 342 | 41.157 | 80.092 | 35.848 | 1.00 | 33.16 |
| 1812 | CB | ARG | B | 342 | 43.206 | 81.530 | 33.534 | 1.00 | 38.57 |
| 1813 | CG | ARG | B | 342 | 44.219 | 81.279 | 32.411 | 1.00 | 39.10 |
| 1814 | CD | ARG | B | 342 | 44.236 | 82.409 | 31.388 | 1.00 | 43.21 |
| 1815 | NE | ARG | B | 342 | 43.056 | 82.369 | 30.530 | 1.00 | 45.40 |
| 1816 | CZ | ARG | B | 342 | 42.886 | 81.514 | 29.527 | 1.00 | 48.22 |
| 1817 | NH1 | ARG | B | 342 | 43.823 | 80.623 | 29.240 | 1.00 | 52.78 |
| 1818 | NH2 | ARG | B | 342 | 41.769 | 81.541 | 28.814 | 1.00 | 54.34 |
| 1819 | N | PRO | B | 343 | 42.488 | 81.757 | 36.530 | 1.00 | 33.03 |
| 1820 | CA | PRO | B | 343 | 41.560 | 82.103 | 37.619 | 1.00 | 34.14 |
| 1821 | C | PRO | B | 343 | 40.261 | 82.693 | 37.065 | 1.00 | 34.06 |
| 1822 | O | PRO | B | 343 | 40.259 | 83.298 | 35.994 | 1.00 | 35.44 |
| 1823 | CB | PRO | B | 343 | 42.334 | 83.152 | 38.433 | 1.00 | 33.07 |
| 1824 | CG | PRO | B | 343 | 43.789 | 82.900 | 38.075 | 1.00 | 35.06 |
| 1825 | CD | PRO | B | 343 | 43.715 | 82.571 | 36.601 | 1.00 | 33.27 |
| 1826 | N | SER | B | 344 | 39.155 | 82.520 | 37.775 | 1.00 | 32.17 |
| 1827 | CA | SER | B | 344 | 37.909 | 83.110 | 37.308 | 1.00 | 31.75 |
| 1828 | C | SER | B | 344 | 37.983 | 84.586 | 37.686 | 1.00 | 32.07 |
| 1829 | O | SER | B | 344 | 38.485 | 84.940 | 38.756 | 1.00 | 30.21 |
| 1830 | CB | SER | B | 344 | 36.693 | 82.456 | 37.982 | 1.00 | 32.20 |
| 1831 | OG | SER | B | 344 | 36.487 | 82.935 | 39.301 | 1.00 | 32.50 |
| 1832 | N | PRO | B | 345 | 37.512 | 85.473 | 36.800 | 1.00 | 32.31 |
| 1833 | CA | PRO | B | 345 | 37.561 | 86.899 | 37.125 | 1.00 | 30.84 |
| 1834 | C | PRO | B | 345 | 36.890 | 87.219 | 38.471 | 1.00 | 29.64 |
| 1835 | O | PRO | B | 345 | 37.344 | 88.094 | 39.209 | 1.00 | 30.14 |
| 1836 | CB | PRO | B | 345 | 36.859 | 87.545 | 35.926 | 1.00 | 30.96 |
| 1837 | CG | PRO | B | 345 | 37.299 | 86.659 | 34.793 | 1.00 | 33.18 |
| 1838 | CD | PRO | B | 345 | 37.143 | 85.261 | 35.387 | 1.00 | 33.65 |
| 1839 | N | PHE | B | 346 | 35.831 | 86.497 | 38.808 | 1.00 | 28.34 |
| 1840 | CA | PHE | B | 346 | 35.153 | 86.756 | 40.066 | 1.00 | 31.46 |
| 1841 | C | PHE | B | 346 | 36.078 | 86.515 | 41.269 | 1.00 | 32.46 |
| 1842 | O | PHE | B | 346 | 36.135 | 87.331 | 42.193 | 1.00 | 32.59 |
| 1843 | CB | PHE | B | 346 | 33.894 | 85.889 | 40.184 | 1.00 | 32.72 |
| 1844 | CG | PHE | B | 346 | 33.218 | 85.987 | 41.521 | 1.00 | 36.34 |
| 1845 | CD1 | PHE | B | 346 | 32.619 | 87.170 | 41.928 | 1.00 | 40.58 |
| 1846 | CD2 | PHE | B | 346 | 33.189 | 84.898 | 42.378 | 1.00 | 36.76 |
| 1847 | CE1 | PHE | B | 346 | 31.991 | 87.267 | 43.167 | 1.00 | 38.98 |
| 1848 | CE2 | PHE | B | 346 | 32.563 | 84.985 | 43.618 | 1.00 | 40.15 |
| 1849 | CZ | PHE | B | 346 | 31.963 | 86.175 | 44.014 | 1.00 | 38.60 |
| 1850 | N | ASP | B | 347 | 36.803 | 85.397 | 41.250 | 1.00 | 32.92 |
| 1851 | CA | ASP | B | 347 | 37.723 | 85.066 | 42.336 | 1.00 | 33.58 |
| 1852 | C | ASP | B | 347 | 38.896 | 86.045 | 42.367 | 1.00 | 34.26 |
| 1853 | O | ASP | B | 347 | 39.388 | 86.408 | 43.434 | 1.00 | 32.29 |
| 1854 | CB | ASP | B | 347 | 38.251 | 83.633 | 42.179 | 1.00 | 28.72 |
| 1855 | CG | ASP | B | 347 | 37.236 | 82.592 | 42.579 | 1.00 | 39.62 |
| 1856 | OD1 | ASP | B | 347 | 37.515 | 81.375 | 42.429 | 1.00 | 42.08 |
| 1857 | OD2 | ASP | B | 347 | 36.151 | 82.991 | 43.054 | 1.00 | 46.23 |
| 1858 | N | LEU | B | 348 | 39.324 | 86.480 | 41.189 | 1.00 | 34.31 |
| 1859 | CA | LEU | B | 348 | 40.441 | 87.403 | 41.064 | 1.00 | 37.70 |
| 1860 | C | LEU | B | 348 | 40.159 | 88.858 | 41.471 | 1.00 | 38.98 |
| 1861 | O | LEU | B | 348 | 40.949 | 89.463 | 42.198 | 1.00 | 38.11 |
| 1862 | CB | LEU | B | 348 | 40.959 | 87.380 | 39.618 | 1.00 | 40.66 |
| 1863 | CG | LEU | B | 348 | 42.159 | 88.267 | 39.260 | 1.00 | 41.99 |
| 1864 | CD1 | LEU | B | 348 | 43.388 | 87.862 | 40.095 | 1.00 | 42.77 |
| 1865 | CD2 | LEU | B | 348 | 42.469 | 88.124 | 37.759 | 1.00 | 41.00 |
| 1866 | N | PHE | B | 349 | 39.040 | 89.417 | 41.017 | 1.00 | 39.68 |
| 1867 | CA | PHE | B | 349 | 38.725 | 90.819 | 41.306 | 1.00 | 42.09 |
| 1868 | C | PHE | B | 349 | 37.717 | 91.134 | 42.405 | 1.00 | 45.09 |
| 1869 | O | PHE | B | 349 | 37.821 | 92.169 | 43.062 | 1.00 | 48.27 |
| 1870 | CB | PHE | B | 349 | 38.272 | 91.526 | 40.029 | 1.00 | 37.36 |
| 1871 | CG | PHE | B | 349 | 39.244 | 91.411 | 38.900 | 1.00 | 36.93 |
| 1872 | CD1 | PHE | B | 349 | 38.976 | 90.579 | 37.818 | 1.00 | 38.16 |
| 1873 | CD2 | PHE | B | 349 | 40.444 | 92.113 | 38.926 | 1.00 | 38.04 |
| 1874 | CE1 | PHE | B | 349 | 39.885 | 90.445 | 36.781 | 1.00 | 38.11 |
| 1875 | CE2 | PHE | B | 349 | 41.369 | 91.986 | 37.887 | 1.00 | 37.58 |
| 1876 | CZ | PHE | B | 349 | 41.089 | 91.151 | 36.817 | 1.00 | 40.39 |
| 1877 | N | ILE | B | 350 | 36.726 | 90.277 | 42.600 | 1.00 | 46.66 |
| 1878 | CA | ILE | B | 350 | 35.746 | 90.548 | 43.639 | 1.00 | 48.94 |
| 1879 | C | ILE | B | 350 | 36.200 | 89.905 | 44.950 | 1.00 | 51.15 |
| 1880 | O | ILE | B | 350 | 36.505 | 90.593 | 45.921 | 1.00 | 50.51 |
| 1881 | CB | ILE | B | 350 | 34.359 | 90.008 | 43.240 | 1.00 | 48.52 |
| 1882 | CG1 | ThE | B | 350 | 34.038 | 90.428 | 41.801 | 1.00 | 49.25 |
| 1883 | CG2 | ILE | B | 350 | 33.300 | 90.548 | 44.177 | 1.00 | 49.68 |
| 1884 | CD1 | ILE | B | 350 | 34.242 | 91.911 | 41.533 | 1.00 | 46.80 |
| 1885 | N | ARG | B | 351 | 36.265 | 88.579 | 44.945 | 1.00 | 52.64 |
| 1886 | CA | ARG | B | 351 | 36.669 | 87.793 | 46.100 | 1.00 | 53.76 |
| 1887 | C | ARG | B | 351 | 38.114 | 88.090 | 46.519 | 1.00 | 53.61 |
| 1888 | O | ARG | B | 351 | 38.436 | 88.094 | 47.703 | 1.00 | 53.55 |
| 1889 | CB | ARG | B | 351 | 36.505 | 86.313 | 45.759 | 1.00 | 56.64 |
| 1890 | CG | ARG | B | 351 | 36.016 | 85.434 | 46.894 | 1.00 | 62.18 |
| 1891 | CD | ARG | B | 351 | 35.073 | 84.375 | 46.348 | 1.00 | 66.96 |
| 1892 | NE | ARG | B | 351 | 34.958 | 83.211 | 47.219 | 1.00 | 71.13 |
| 1893 | CZ | ARG | B | 351 | 35.957 | 82.371 | 47.470 | 1.00 | 76.72 |
| 1894 | NH1 | ARG | B | 351 | 37.145 | 82.573 | 46.916 | 1.00 | 78.73 |
| 1895 | NH2 | ARG | B | 351 | 35.771 | 81.325 | 48.268 | 1.00 | 77.63 |
| 1896 | N | LYS | B | 352 | 38.974 | 88.347 | 45.540 | 1.00 | 53.86 |
| 1897 | CA | LYS | B | 352 | 40.385 | 88.646 | 45.788 | 1.00 | 54.12 |
| 1898 | C | LYS | B | 352 | 41.172 | 87.449 | 46.328 | 1.00 | 52.02 |
| 1899 | O | LYS | B | 352 | 42.078 | 87.604 | 47.146 | 1.00 | 51.77 |
| 1900 | CB | LYS | B | 352 | 40.525 | 89.825 | 46.753 | 1.00 | 57.77 |
| 1901 | CG | LYS | B | 352 | 39.911 | 91.124 | 46.254 | 1.00 | 62.15 |
| 1902 | CB | LYS | B | 352 | 40.161 | 92.252 | 47.254 | 1.00 | 71.06 |
| 1903 | CE | LYS | B | 352 | 39.351 | 93.502 | 46.929 | 1.00 | 74.25 |
| 1904 | NZ | LYS | B | 352 | 37.893 | 93.307 | 47.185 | 1.00 | 78.35 |
| 1905 | N | SER | B | 353 | 40.811 | 86.258 | 45.867 | 1.00 | 48.01 |
| 1906 | CA | SER | B | 353 | 41.488 | 85.032 | 46.268 | 1.00 | 45.29 |
| 1907 | C | SER | B | 353 | 41.446 | 84.056 | 45.084 | 1.00 | 40.67 |
| 1908 | O | SER | B | 353 | 40.725 | 83.054 | 45.106 | 1.00 | 40.76 |
| 1909 | CB | SER | B | 353 | 40.809 | 84.423 | 47.498 | 1.00 | 47.97 |
| 1910 | OG | SER | B | 353 | 39.445 | 84.152 | 47.247 | 1.00 | 56.20 |
| 1911 | N | PRO | B | 354 | 42.225 | 84.356 | 44.030 | 1.00 | 36.53 |
| 1912 | CA | PRO | B | 354 | 42.361 | 83.596 | 42.781 | 1.00 | 32.68 |
| 1913 | C | PRO | B | 354 | 42.940 | 82.201 | 42.961 | 1.00 | 31.88 |
| 1914 | O | PRO | B | 354 | 43.813 | 81.985 | 43.798 | 1.00 | 29.76 |
| 1915 | CB | PRO | B | 354 | 43.306 | 84.453 | 41.940 | 1.00 | 30.29 |
| 1916 | CG | PRO | B | 354 | 43.260 | 85.801 | 42.587 | 1.00 | 41.68 |
| 1917 | CD | PRO | B | 354 | 43.155 | 85.494 | 44.035 | 1.00 | 33.29 |
| 1918 | N | THR | B | 355 | 42.446 | 81.258 | 42.168 | 1.00 | 30.03 |
| 1919 | CA | THR | B | 355 | 42.956 | 79.896 | 42.198 | 1.00 | 29.70 |
| 1920 | C | THR | B | 355 | 43.022 | 79.364 | 40.774 | 1.00 | 30.82 |
| 1921 | O | THR | B | 355 | 42.302 | 79.827 | 39.887 | 1.00 | 30.67 |
| 1922 | CB | THR | B | 355 | 42.042 | 78.931 | 42.964 | 1.00 | 32.14 |
| 1923 | OG1 | THR | B | 355 | 40.815 | 78.794 | 42.240 | 1.00 | 29.97 |
| 1924 | CG2 | THR | B | 355 | 41.763 | 79.425 | 44.393 | 1.00 | 31.76 |
| 1925 | N | ILE | B | 356 | 43.907 | 78.405 | 40.542 | 1.00 | 30.53 |
| 1926 | CA | ILE | B | 356 | 43.969 | 77.784 | 39.232 | 1.00 | 31.56 |
| 1927 | C | ILE | B | 356 | 43.740 | 76.321 | 39.525 | 1.00 | 31.79 |
| 1928 | O | ILE | B | 356 | 44.039 | 75.848 | 40.625 | 1.00 | 30.35 |
| 1929 | CB | ILE | B | 356 | 45.315 | 78.016 | 38.517 | 1.00 | 30.05 |
| 1930 | CG1 | ILE | B | 356 | 46.467 | 77.499 | 39.371 | 1.00 | 31.32 |
| 1931 | CG2 | ILE | B | 356 | 45.469 | 79.508 | 38.210 | 1.00 | 31.27 |
| 1932 | CD1 | ILE | B | 356 | 47.828 | 77.757 | 38.763 | 1.00 | 36.89 |
| 1933 | N | THR | B | 357 | 43.198 | 75.605 | 38.550 | 1.00 | 33.31 |
| 1934 | CA | THR | B | 357 | 42.881 | 74.205 | 38.751 | 1.00 | 33.68 |
| 1935 | C | THR | B | 357 | 43.403 | 73.318 | 37.644 | 1.00 | 35.47 |
| 1936 | O | THR | B | 357 | 43.228 | 73.607 | 36.459 | 1.00 | 36.65 |
| 1937 | CB | THR | B | 357 | 41.335 | 74.011 | 38.855 | 1.00 | 33.81 |
| 1938 | OG1 | THR | B | 357 | 40.844 | 74.683 | 40.023 | 1.00 | 33.71 |
| 1939 | CG2 | THR | B | 357 | 40.968 | 72.535 | 38.921 | 1.00 | 29.63 |
| 1940 | N | CYS | B | 358 | 44.034 | 72.228 | 38.048 | 1.00 | 35.41 |
| 1941 | CA | CYS | B | 358 | 44.561 | 71.254 | 37.110 | 1.00 | 38.12 |
| 1942 | C | CYS | B | 358 | 43.570 | 70.093 | 37.165 | 1.00 | 37.77 |
| 1943 | O | CYS | B | 358 | 43.395 | 69.473 | 38.210 | 1.00 | 36.67 |
| 1944 | CB | CYS | B | 358 | 45.945 | 70.789 | 37.568 | 1.00 | 40.37 |
| 1945 | SG | CYS | B | 358 | 46.871 | 69.769 | 36.374 | 1.00 | 49.83 |
| 1946 | N | LEU | B | 359 | 42.909 | 69.822 | 36.046 | 1.00 | 39.77 |
| 1947 | CA | LEU | B | 359 | 41.932 | 68.747 | 35.973 | 1.00 | 42.86 |
| 1948 | C | LEU | B | 359 | 42.432 | 67.593 | 35.123 | 1.00 | 46.01 |
| 1949 | O | LEU | B | 359 | 42.748 | 67.769 | 33.948 | 1.00 | 47.43 |
| 1950 | CB | LEU | B | 359 | 40.612 | 69.265 | 35.389 | 1.00 | 42.74 |
| 1951 | CG | LEU | B | 359 | 39.604 | 68.208 | 34.904 | 1.00 | 45.03 |
| 1952 | CB1 | LEU | B | 359 | 39.152 | 67.338 | 36.057 | 1.00 | 47.59 |
| 1953 | CD2 | LEU | B | 359 | 38.403 | 68.889 | 34.262 | 1.00 | 47.51 |
| 1954 | N | VAL | B | 360 | 42.493 | 66.411 | 35.721 | 1.00 | 48.26 |
| 1955 | CA | VAL | B | 360 | 42.931 | 65.220 | 35.010 | 1.00 | 52.34 |
| 1956 | C | VAL | B | 360 | 41.765 | 64.243 | 34.883 | 1.00 | 55.48 |
| 1957 | O | VAL | B | 360 | 41.092 | 63.928 | 35.866 | 1.00 | 55.57 |
| 1958 | CB | VAL | B | 360 | 44.100 | 64.527 | 35.747 | 1.00 | 53.36 |
| 1959 | CG1 | VAL | B | 360 | 44.481 | 63.240 | 35.029 | 1.00 | 51.62 |
| 1960 | CG2 | VAL | B | 360 | 45.299 | 65.467 | 35.816 | 1.00 | 51.77 |
| 1961 | N | VAL | B | 361 | 41.520 | 63.773 | 33.665 | 1.00 | 58.92 |
| 1962 | CA | VAL | B | 361 | 40.433 | 62.834 | 33.416 | 1.00 | 63.13 |
| 1963 | C | VAL | B | 361 | 40.993 | 61.506 | 32.926 | 1.00 | 67.77 |
| 1964 | O | VAL | B | 361 | 41.738 | 61.466 | 31.946 | 1.00 | 68.38 |
| 1965 | CB | VAL | B | 361 | 39.442 | 63.377 | 32.353 | 1.00 | 60.50 |
| 1966 | CG1 | VAL | B | 361 | 38.358 | 62.355 | 32.086 | 1.00 | 56.92 |
| 1967 | CG2 | VAL | B | 361 | 38.814 | 64.671 | 32.830 | 1.00 | 56.10 |
| 1968 | N | ASP | B | 362 | 40.635 | 60.426 | 33.615 | 1.00 | 72.90 |
| 1969 | CA | ASP | B | 362 | 41.098 | 59.085 | 33.259 | 1.00 | 78.30 |
| 1970 | C | ASP | B | 362 | 39.904 | 58.244 | 32.816 | 1.00 | 81.68 |
| 1971 | O | ASP | B | 362 | 39.168 | 57.709 | 33.648 | 1.00 | 81.72 |
| 1972 | CB | ASP | B | 362 | 41.771 | 58.415 | 34.462 | 1.00 | 80.22 |
| 1973 | CG | ASP | B | 362 | 42.530 | 57.156 | 34.080 | 1.00 | 84.67 |
| 1974 | OD1 | ASP | B | 362 | 42.113 | 56.478 | 33.117 | 1.00 | 87.54 |
| 1975 | OD2 | ASP | B | 362 | 43.537 | 56.837 | 34.749 | 1.00 | 87.19 |
| 1976 | N | LEU | B | 363 | 39.723 | 58.126 | 31.503 | 1.00 | 85.84 |
| 1977 | CA | LEU | B | 363 | 38.614 | 57.366 | 30.935 | 1.00 | 89.95 |
| 1978 | C | LEU | B | 363 | 38.562 | 55.923 | 31.428 | 1.00 | 92.20 |
| 1979 | O | LEU | B | 363 | 37.483 | 55.386 | 31.668 | 1.00 | 92.55 |
| 1980 | CB | LEU | B | 363 | 38.694 | 57.390 | 29.409 | 1.00 | 91.66 |
| 1981 | CG | LEU | B | 363 | 38.719 | 58.777 | 28.758 | 1.00 | 92.93 |
| 1982 | CB1 | LEU | B | 363 | 38.813 | 58.624 | 27.246 | 1.00 | 95.41 |
| 1983 | CD2 | LEU | B | 363 | 37.471 | 59.551 | 29.137 | 1.00 | 92.73 |
| 1984 | N | ALA | B | 364 | 39.726 | 55.296 | 31.573 | 1.00 | 94.76 |
| 1985 | CA | ALA | B | 364 | 39.801 | 53.916 | 32.051 | 1.00 | 97.66 |
| 1986 | C | ALA | B | 364 | 40.794 | 53.831 | 33.210 | 1.00 | 99.72 |
| 1987 | O | ALA | B | 364 | 41.998 | 53.996 | 33.021 | 1.00 | 100.44 |
| 1988 | CB | ALA | B | 364 | 40.235 | 52.996 | 30.921 | 1.00 | 97.99 |
| 1989 | N | PRO | B | 365 | 40.297 | 53.577 | 34.429 | 1.00 | 101.15 |
| 1990 | CA | PRO | B | 365 | 41.173 | 53.484 | 35.602 | 1.00 | 102.25 |
| 1991 | C | PRO | B | 365 | 41.895 | 52.163 | 35.855 | 1.00 | 103.43 |
| 1992 | O | PRO | B | 365 | 41.274 | 51.103 | 35.934 | 1.00 | 104.19 |
| 1993 | CB | PRO | B | 365 | 40.237 | 53.829 | 36.761 | 1.00 | 101.91 |
| 1994 | CG | PRO | B | 365 | 39.121 | 54.617 | 36.098 | 1.00 | 102.27 |
| 1995 | CB | PRO | B | 365 | 38.910 | 53.833 | 34.846 | 1.00 | 101.40 |
| 1996 | N | SER | B | 366 | 43.215 | 52.256 | 35.979 | 1.00 | 104.18 |
| 1997 | CA | SER | B | 366 | 44.088 | 51.132 | 36.299 | 1.00 | 104.43 |
| 1998 | C | SER | B | 366 | 44.573 | 51.676 | 37.637 | 1.00 | 104.05 |
| 1999 | O | SER | B | 366 | 44.888 | 52.864 | 37.727 | 1.00 | 104.36 |
| 2000 | CB | SER | B | 366 | 45.249 | 51.055 | 35.312 | 1.00 | 105.50 |
| 2001 | OG | SER | B | 366 | 46.040 | 52.223 | 35.413 | 1.00 | 106.70 |
| 2002 | N | LYS | B | 367 | 44.645 | 50.861 | 38.682 | 1.00 | 103.19 |
| 2003 | CA | LYS | B | 367 | 45.041 | 51.462 | 39.944 | 1.00 | 102.08 |
| 2004 | C | LYS | B | 367 | 46.491 | 51.630 | 40.367 | 1.00 | 100.74 |
| 2005 | O | LYS | B | 367 | 47.234 | 50.683 | 40.640 | 1.00 | 101.05 |
| 2006 | CB | LYS | B | 367 | 44.236 | 50.868 | 41.102 | 1.00 | 103.30 |
| 2007 | CG | LYS | B | 367 | 44.343 | 49.384 | 41.345 | 1.00 | 105.00 |
| 2008 | CD | LYS | B | 367 | 43.374 | 49.046 | 42.467 | 1.00 | 104.80 |
| 2009 | CE | LYS | B | 367 | 43.467 | 47.610 | 42.930 | 1.00 | 104.70 |
| 2010 | NZ | LYS | B | 367 | 42.524 | 47.386 | 44.066 | 1.00 | 105.18 |
| 2011 | N | GLY | B | 368 | 46.839 | 52.909 | 40.422 | 1.00 | 98.70 |
| 2012 | CA | GLY | B | 368 | 48.136 | 53.408 | 40.828 | 1.00 | 95.62 |
| 2013 | C | GLY | B | 368 | 47.700 | 54.801 | 41.244 | 1.00 | 93.12 |
| 2014 | O | GLY | B | 368 | 46.538 | 55.143 | 41.035 | 1.00 | 93.17 |
| 2015 | N | THR | B | 369 | 48.569 | 55.617 | 41.823 | 1.00 | 90.15 |
| 2016 | CA | THR | B | 369 | 48.125 | 56.948 | 42.220 | 1.00 | 86.41 |
| 2017 | C | THR | B | 369 | 48.420 | 57.977 | 41.136 | 1.00 | 83.04 |
| 2018 | O | THR | B | 369 | 49.402 | 57.854 | 40.401 | 1.00 | 82.54 |
| 2019 | CB | THR | B | 369 | 48.792 | 57.397 | 43.541 | 1.00 | 87.73 |
| 2020 | OG1 | THR | B | 369 | 50.217 | 57.422 | 43.379 | 1.00 | 88.60 |
| 2021 | CG2 | THR | B | 369 | 48.423 | 56.443 | 44.671 | 1.00 | 86.64 |
| 2022 | N | VAL | B | 370 | 47.552 | 58.980 | 41.027 | 1.00 | 79.07 |
| 2023 | CA | VAL | B | 370 | 47.729 | 60.041 | 40.042 | 1.00 | 74.46 |
| 2024 | C | VAL | B | 370 | 48.275 | 61.261 | 40.768 | 1.00 | 72.21 |
| 2025 | O | VAL | B | 370 | 47.602 | 61.838 | 41.618 | 1.00 | 71.35 |
| 2026 | CB | VAL | B | 370 | 46.401 | 60.420 | 39.368 | 1.00 | 73.33 |
| 2027 | CG1 | VAL | B | 370 | 46.667 | 61.346 | 38.196 | 1.00 | 70.92 |
| 2028 | CG2 | VAL | B | 370 | 45.674 | 59.173 | 38.915 | 1.00 | 69.77 |
| 2029 | N | ASN | B | 371 | 49.498 | 61.653 | 40.426 | 1.00 | 70.13 |
| 2030 | CA | ASN | B | 371 | 50.137 | 62.791 | 41.073 | 1.00 | 68.15 |
| 2031 | C | ASN | B | 371 | 50.157 | 64.079 | 40.262 | 1.00 | 65.65 |
| 2032 | O | ASN | B | 371 | 50.352 | 64.067 | 39.046 | 1.00 | 64.64 |
| 2033 | CB | ASN | B | 371 | 51.564 | 62.421 | 41.469 | 1.00 | 70.12 |
| 2034 | CG | ASN | B | 371 | 51.605 | 61.330 | 42.508 | 1.00 | 73.56 |
| 2035 | OD1 | ASN | B | 371 | 51.153 | 61.519 | 43.638 | 1.00 | 75.10 |
| 2036 | ND2 | ASN | B | 371 | 52.137 | 60.173 | 42.131 | 1.00 | 74.76 |
| 2037 | N | LEU | B | 372 | 49.949 | 65.192 | 40.959 | 1.00 | 62.66 |
| 2038 | CA | LEU | B | 372 | 49.959 | 66.512 | 40.346 | 1.00 | 59.53 |
| 2039 | C | LEU | B | 372 | 50.927 | 67.374 | 41.137 | 1.00 | 56.68 |
| 2040 | O | LEU | B | 372 | 50.788 | 67.529 | 42.347 | 1.00 | 56.70 |
| 2041 | CB | LEU | B | 372 | 48.560 | 67.141 | 40.372 | 1.00 | 59.44 |
| 2042 | CG | LEU | B | 372 | 47.428 | 66.383 | 39.678 | 1.00 | 60.55 |
| 2043 | CD1 | LEU | B | 372 | 46.214 | 67.277 | 39.579 | 1.00 | 61.28 |
| 2044 | CD2 | LEU | B | 372 | 47.861 | 65.956 | 38.293 | 1.00 | 62.83 |
| 2045 | N | THR | B | 373 | 51.911 | 67.931 | 40.446 | 1.00 | 53.76 |
| 2046 | CA | THR | B | 373 | 52.915 | 68.766 | 41.085 | 1.00 | 51.64 |
| 2047 | C | THR | B | 373 | 52.850 | 70.180 | 40.512 | 1.00 | 48.20 |
| 2048 | O | THR | B | 373 | 52.801 | 70.355 | 39.296 | 1.00 | 46.65 |
| 2049 | CB | THR | B | 373 | 54.325 | 68.201 | 40.832 | 1.00 | 53.15 |
| 2050 | OG1 | THR | B | 373 | 54.324 | 66.789 | 41.073 | 1.00 | 57.77 |
| 2051 | CG2 | THR | B | 373 | 55.339 | 68.871 | 41.747 | 1.00 | 53.75 |
| 2052 | N | THR | B | 374 | 52.851 | 71.181 | 41.385 | 1.00 | 45.34 |
| 2053 | CA | THR | B | 374 | 52.799 | 72.575 | 40.942 | 1.00 | 44.94 |
| 2054 | C | THR | B | 374 | 54.161 | 73.259 | 41.041 | 1.00 | 43.68 |
| 2055 | O | THR | B | 374 | 54.945 | 72.964 | 41.938 | 1.00 | 42.67 |
| 2056 | CB | THR | B | 374 | 51.805 | 73.391 | 41.784 | 1.00 | 41.63 |
| 2057 | CG | THR | B | 374 | 50.371 | 72.977 | 41.657 | 1.00 | 41.50 |
| 2058 | CD1 | THR | B | 374 | 49.688 | 72.124 | 42.477 | 1.00 | 39.67 |
| 2059 | CD2 | THR | B | 374 | 49.441 | 73.395 | 40.648 | 1.00 | 35.69 |
| 2060 | NE1 | THR | B | 374 | 48.392 | 71.988 | 42.041 | 1.00 | 35.06 |
| 2061 | CE2 | THR | B | 374 | 48.214 | 72.756 | 40.922 | 1.00 | 34.53 |
| 2062 | CE3 | THR | B | 374 | 49.528 | 74.247 | 39.542 | 1.00 | 32.37 |
| 2063 | CZ2 | THR | B | 374 | 47.079 | 72.943 | 40.129 | 1.00 | 35.43 |
| 2064 | CZ3 | THR | B | 374 | 48.401 | 74.434 | 38.751 | 1.00 | 36.11 |
| 2065 | CH2 | THR | B | 374 | 47.190 | 73.782 | 39.050 | 1.00 | 37.51 |
| 2066 | N | SER | B | 375 | 54.430 | 74.179 | 40.119 | 1.00 | 43.53 |
| 2067 | CA | SER | B | 375 | 55.678 | 74.934 | 40.140 | 1.00 | 43.57 |
| 2068 | C | SER | B | 375 | 55.511 | 76.270 | 39.438 | 1.00 | 42.59 |
| 2069 | O | SER | B | 375 | 54.669 | 76.411 | 38.550 | 1.00 | 43.68 |
| 2070 | CB | SER | B | 375 | 56.820 | 74.153 | 39.474 | 1.00 | 45.03 |
| 2071 | OG | SER | B | 375 | 56.691 | 74.118 | 38.066 | 1.00 | 45.66 |
| 2072 | N | ARG | B | 376 | 56.299 | 77.251 | 39.866 | 1.00 | 40.79 |
| 2073 | CA | ARG | B | 376 | 56.279 | 78.569 | 39.266 | 1.00 | 40.12 |
| 2074 | C | ARG | B | 376 | 57.498 | 78.689 | 38.351 | 1.00 | 40.59 |
| 2075 | O | ARG | B | 376 | 58.618 | 78.393 | 38.752 | 1.00 | 40.90 |
| 2076 | CB | ARG | B | 376 | 56.345 | 79.653 | 40.334 | 1.00 | 40.03 |
| 2077 | CG | ARG | B | 376 | 55.132 | 79.745 | 41.237 | 1.00 | 43.20 |
| 2078 | CD | ARG | B | 376 | 54.883 | 81.198 | 41.599 | 1.00 | 41.05 |
| 2079 | NE | ARG | B | 376 | 55.171 | 81.480 | 42.992 | 1.00 | 50.71 |
| 2080 | CZ | ARG | B | 376 | 55.300 | 82.705 | 43.488 | 1.00 | 53.97 |
| 2081 | NH1 | ARG | B | 376 | 55.172 | 83.758 | 42.693 | 1.00 | 51.88 |
| 2082 | NH2 | ARG | B | 376 | 55.542 | 82.874 | 44.782 | 1.00 | 55.55 |
| 2083 | N | ALA | B | 377 | 57.281 | 79.129 | 37.122 | 1.00 | 40.20 |
| 2084 | CA | ALA | B | 377 | 58.382 | 79.273 | 36.181 | 1.00 | 40.25 |
| 2085 | C | ALA | B | 377 | 59.500 | 80.130 | 36.786 | 1.00 | 40.38 |
| 2086 | O | ALA | B | 377 | 60.657 | 79.979 | 36.422 | 1.00 | 41.16 |
| 2087 | CB | ALA | B | 377 | 57.879 | 79.903 | 34.878 | 1.00 | 33.12 |
| 2088 | N | SER | B | 378 | 59.144 | 81.015 | 37.714 | 1.00 | 40.21 |
| 2089 | CA | SER | B | 378 | 60.109 | 81.903 | 38.354 | 1.00 | 41.95 |
| 2090 | C | SER | B | 378 | 60.906 | 81.188 | 39.436 | 1.00 | 44.92 |
| 2091 | O | SER | B | 378 | 61.866 | 81.744 | 39.966 | 1.00 | 44.28 |
| 2092 | CB | SER | B | 378 | 59.401 | 83.096 | 38.992 | 1.00 | 40.91 |
| 2093 | OG | SER | B | 378 | 58.737 | 82.693 | 40.180 | 1.00 | 40.09 |
| 2094 | N | GLY | B | 379 | 60.493 | 79.967 | 39.768 | 1.00 | 46.93 |
| 2095 | CA | GLY | B | 379 | 61.181 | 79.195 | 40.787 | 1.00 | 49.77 |
| 2096 | C | GLY | B | 379 | 60.716 | 79.512 | 42.196 | 1.00 | 50.60 |
| 2097 | O | GLY | B | 379 | 61.024 | 78.783 | 43.142 | 1.00 | 52.95 |
| 2098 | N | LYS | B | 380 | 59.974 | 80.605 | 42.345 | 1.00 | 53.72 |
| 2099 | CA | LYS | B | 380 | 59.478 | 80.988 | 43.657 | 1.00 | 54.13 |
| 2100 | C | LYS | B | 380 | 58.564 | 79.911 | 44.255 | 1.00 | 48.16 |
| 2101 | O | LYS | B | 380 | 58.087 | 79.008 | 43.556 | 1.00 | 45.65 |
| 2102 | CB | LYS | B | 380 | 58.752 | 82.338 | 43.583 | 1.00 | 62.44 |
| 2103 | CG | LYS | B | 380 | 59.692 | 83.522 | 43.390 | 1.00 | 67.72 |
| 2104 | CD | LYS | B | 380 | 58.975 | 84.852 | 43.550 | 1.00 | 69.32 |
| 2105 | CE | LYS | B | 380 | 59.956 | 86.013 | 43.481 | 1.00 | 73.14 |
| 2106 | NZ | LYS | B | 380 | 59.280 | 87.333 | 43.633 | 1.00 | 77.37 |
| 2107 | N | PRO | B | 381 | 58.323 | 79.986 | 45.571 | 1.00 | 56.07 |
| 2108 | CA | PRO | B | 381 | 57.472 | 79.014 | 46.265 | 1.00 | 56.46 |
| 2109 | C | PRO | B | 381 | 55.993 | 79.065 | 45.875 | 1.00 | 54.32 |
| 2110 | O | PRO | B | 381 | 55.446 | 80.133 | 45.589 | 1.00 | 56.76 |
| 2111 | CB | PRO | B | 381 | 57.664 | 79.375 | 47.740 | 1.00 | 60.70 |
| 2112 | CG | PRO | B | 381 | 58.978 | 80.119 | 47.767 | 1.00 | 62.01 |
| 2113 | CD | PRO | B | 381 | 58.905 | 80.945 | 46.526 | 1.00 | 59.24 |
| 2114 | N | VAL | B | 382 | 55.356 | 77.899 | 45.866 | 1.00 | 55.74 |
| 2115 | CA | VAL | B | 382 | 53.931 | 77.805 | 45.569 | 1.00 | 55.58 |
| 2116 | C | VAL | B | 382 | 53.247 | 77.408 | 46.880 | 1.00 | 55.56 |
| 2117 | O | VAL | B | 382 | 53.848 | 76.734 | 47.720 | 1.00 | 55.22 |
| 2118 | CB | VAL | B | 382 | 53.620 | 76.726 | 44.493 | 1.00 | 55.39 |
| 2119 | CG1 | VAL | B | 382 | 54.404 | 77.008 | 43.222 | 1.00 | 55.94 |
| 2120 | CG2 | VAL | B | 382 | 53.936 | 75.339 | 45.025 | 1.00 | 53.06 |
| 2121 | N | ASN | B | 383 | 52.000 | 77.827 | 47.055 | 1.00 | 54.38 |
| 2122 | CA | ASN | B | 383 | 51.246 | 77.510 | 48.264 | 1.00 | 54.63 |
| 2123 | C | ASN | B | 383 | 50.757 | 76.068 | 48.290 | 1.00 | 53.85 |
| 2124 | O | ASN | B | 383 | 50.970 | 75.297 | 47.354 | 1.00 | 52.58 |
| 2125 | CB | ASN | B | 383 | 50.034 | 78.429 | 48.382 | 1.00 | 59.07 |
| 2126 | CG | ASN | B | 383 | 50.414 | 79.883 | 48.456 | 1.00 | 64.60 |
| 2127 | OD1 | ASN | B | 383 | 49.576 | 80.764 | 48.269 | 1.00 | 70.14 |
| 2128 | ND2 | ASN | B | 383 | 51.685 | 80.150 | 48.738 | 1.00 | 71.07 |
| 2129 | N | HIS | B | 384 | 50.094 | 75.710 | 49.381 | 1.00 | 53.46 |
| 2130 | CA | HIS | B | 384 | 49.543 | 74.371 | 49.526 | 1.00 | 52.22 |
| 2131 | C | HIS | B | 384 | 48.298 | 74.274 | 48.640 | 1.00 | 49.10 |
| 2132 | O | HIS | B | 384 | 47.495 | 75.200 | 48.585 | 1.00 | 45.53 |
| 2133 | CB | HIS | B | 384 | 49.183 | 74.111 | 50.993 | 1.00 | 59.11 |
| 2134 | CG | HIS | B | 384 | 50.377 | 73.924 | 51.878 | 1.00 | 62.16 |
| 2135 | ND1 | HIS | B | 384 | 51.243 | 72.862 | 51.739 | 1.00 | 69.15 |
| 2136 | CD2 | HIS | B | 384 | 50.855 | 74.669 | 52.903 | 1.00 | 70.28 |
| 2137 | CE1 | HIS | B | 384 | 52.206 | 72.960 | 52.639 | 1.00 | 75.01 |
| 2138 | NE2 | HIS | B | 384 | 51.995 | 74.047 | 53.358 | 1.00 | 74.58 |
| 2139 | N | SER | B | 385 | 48.146 | 73.152 | 47.949 | 1.00 | 45.07 |
| 2140 | CA | SER | B | 385 | 47.017 | 72.959 | 47.060 | 1.00 | 44.61 |
| 2141 | C | SER | B | 385 | 45.973 | 71.983 | 47.604 | 1.00 | 44.58 |
| 2142 | O | SER | B | 385 | 46.244 | 71.220 | 48.521 | 1.00 | 44.21 |
| 2143 | CB | SER | B | 385 | 47.524 | 72.487 | 45.691 | 1.00 | 44.61 |
| 2144 | OG | SER | B | 385 | 48.310 | 71.307 | 45.789 | 1.00 | 41.13 |
| 2145 | N | THR | B | 386 | 44.773 | 72.025 | 47.035 | 1.00 | 44.32 |
| 2146 | CA | THR | B | 386 | 43.684 | 71.150 | 47.446 | 1.00 | 44.35 |
| 2147 | C | THR | B | 386 | 43.444 | 70.113 | 46.363 | 1.00 | 44.09 |
| 2148 | O | THR | B | 386 | 43.274 | 70.451 | 45.189 | 1.00 | 44.06 |
| 2149 | CB | THR | B | 386 | 42.374 | 71.936 | 47.680 | 1.00 | 45.20 |
| 2150 | OG1 | THR | B | 386 | 42.555 | 72.856 | 48.762 | 1.00 | 51.89 |
| 2151 | CG2 | THR | B | 386 | 41.232 | 70.984 | 48.036 | 1.00 | 47.20 |
| 2152 | N | ARG | B | 387 | 43.424 | 68.851 | 46.774 | 1.00 | 44.39 |
| 2153 | CA | ARG | B | 387 | 43.222 | 67.730 | 45.867 | 1.00 | 46.68 |
| 2154 | C | ARG | B | 387 | 41.837 | 67.080 | 46.030 | 1.00 | 47.05 |
| 2155 | O | ARG | B | 387 | 41.372 | 66.850 | 47.149 | 1.00 | 46.65 |
| 2156 | CB | ARG | B | 387 | 44.316 | 66.694 | 46.121 | 1.00 | 48.13 |
| 2157 | CG | ARG | B | 387 | 44.270 | 65.488 | 45.210 | 1.00 | 52.77 |
| 2158 | CD | ARG | B | 387 | 45.106 | 64.353 | 45.780 | 1.00 | 56.60 |
| 2159 | NE | ARG | B | 387 | 44.978 | 63.141 | 44.979 | 1.00 | 60.65 |
| 2160 | CZ | ARG | B | 387 | 45.683 | 62.895 | 43.882 | 1.00 | 61.97 |
| 2161 | NH1 | ARG | B | 387 | 46.578 | 63.779 | 43.464 | 1.00 | 64.45 |
| 2162 | NH2 | ARG | B | 387 | 45.469 | 61.781 | 43.189 | 1.00 | 60.54 |
| 2163 | N | LYS | B | 388 | 41.185 | 66.780 | 44.910 | 1.00 | 46.93 |
| 2164 | CA | LYS | B | 388 | 39.870 | 66.143 | 44.933 | 1.00 | 47.51 |
| 2165 | C | LYS | B | 388 | 39.823 | 64.970 | 43.957 | 1.00 | 48.33 |
| 2166 | O | LYS | B | 388 | 40.199 | 65.107 | 42.794 | 1.00 | 46.08 |
| 2167 | CB | LYS | B | 388 | 38.777 | 67.148 | 44.563 | 1.00 | 46.64 |
| 2168 | CG | LYS | B | 388 | 38.691 | 68.326 | 45.497 | 1.00 | 52.75 |
| 2169 | CD | LYS | B | 388 | 37.861 | 69.446 | 44.905 | 1.00 | 58.62 |
| 2170 | CE | LYS | B | 388 | 38.062 | 70.737 | 45.682 | 1.00 | 62.49 |
| 2171 | NZ | LYS | B | 388 | 37.396 | 71.881 | 44.999 | 1.00 | 67.33 |
| 2172 | N | GLU | B | 389 | 39.364 | 63.820 | 44.449 | 1.00 | 50.07 |
| 2173 | CA | GLU | B | 389 | 39.243 | 62.602 | 43.646 | 1.00 | 51.86 |
| 2174 | C | GLU | B | 389 | 37.793 | 62.126 | 43.638 | 1.00 | 52.81 |
| 2175 | O | GLU | B | 389 | 37.192 | 61.917 | 44.695 | 1.00 | 49.21 |
| 2176 | CB | GLU | B | 389 | 40.126 | 61.494 | 44.223 | 1.00 | 54.67 |
| 2177 | CG | GLU | B | 389 | 41.616 | 61.710 | 44.044 | 1.00 | 65.65 |
| 2178 | CD | GLU | B | 389 | 42.441 | 60.608 | 44.688 | 1.00 | 72.66 |
| 2179 | OE1 | GLU | B | 389 | 42.151 | 59.418 | 44.429 | 1.00 | 75.92 |
| 2180 | OE2 | GLU | B | 389 | 43.380 | 60.931 | 45.450 | 1.00 | 75.78 |
| 2181 | N | GLU | B | 390 | 37.229 | 61.948 | 42.449 | 1.00 | 56.06 |
| 2182 | CA | GLU | B | 390 | 35.849 | 61.500 | 42.346 | 1.00 | 61.17 |
| 2183 | C | GLU | B | 390 | 35.629 | 60.454 | 41.265 | 1.00 | 64.70 |
| 2184 | O | GLU | B | 390 | 36.072 | 60.598 | 40.123 | 1.00 | 64.01 |
| 2185 | CB | GLU | B | 390 | 34.927 | 62.692 | 42.107 | 1.00 | 64.35 |
| 2186 | CG | GLU | B | 390 | 33.479 | 62.317 | 41.867 | 1.00 | 74.80 |
| 2187 | CD | GLU | B | 390 | 32.548 | 63.509 | 41.979 | 1.00 | 85.97 |
| 2188 | OE1 | GLU | B | 390 | 32.822 | 64.547 | 41.336 | 1.00 | 92.60 |
| 2189 | OE2 | GLU | B | 390 | 31.541 | 63.403 | 42.712 | 1.00 | 92.18 |
| 2190 | N | LYS | B | 391 | 34.921 | 59.396 | 41.639 | 1.00 | 68.18 |
| 2191 | CA | LYS | B | 391 | 34.640 | 58.312 | 40.712 | 1.00 | 71.58 |
| 2192 | C | LYS | B | 391 | 33.215 | 58.302 | 40.180 | 1.00 | 76.11 |
| 2193 | O | LYS | B | 391 | 32.265 | 58.673 | 40.869 | 1.00 | 75.89 |
| 2194 | CB | LYS | B | 391 | 34.946 | 56.973 | 41.372 | 1.00 | 67.28 |
| 2195 | N | GLN | B | 392 | 33.092 | 57.876 | 38.932 | 1.00 | 81.85 |
| 2196 | CA | CLN | B | 392 | 31.814 | 57.746 | 38.254 | 1.00 | 88.38 |
| 2197 | C | CLN | B | 392 | 32.026 | 56.482 | 37.443 | 1.00 | 92.30 |
| 2198 | O | GLN | B | 392 | 32.752 | 56.500 | 36.452 | 1.00 | 93.25 |
| 2199 | CB | GLN | B | 392 | 31.555 | 58.938 | 37.323 | 1.00 | 85.87 |
| 2200 | CG | GLN | B | 392 | 30.422 | 59.848 | 37.782 | 1.00 | 90.76 |
| 2201 | CD | GLN | B | 392 | 30.027 | 60.873 | 36.731 | 1.00 | 93.39 |
| 2202 | OE1 | GLN | B | 392 | 29.687 | 60.524 | 35.598 | 1.00 | 92.93 |
| 2203 | NE2 | GLN | B | 392 | 30.062 | 62.147 | 37.107 | 1.00 | 93.97 |
| 2204 | N | ARG | B | 393 | 31.426 | 55.379 | 37.880 | 1.00 | 96.24 |
| 2205 | CA | ARG | B | 393 | 31.589 | 54.108 | 37.184 | 1.00 | 100.53 |
| 2206 | C | ARG | B | 393 | 31.250 | 54.188 | 35.697 | 1.00 | 102.30 |
| 2207 | O | ARG | B | 393 | 31.273 | 53.178 | 34.993 | 1.00 | 101.15 |
| 2208 | CB | ARG | B | 393 | 30.747 | 53.021 | 37.855 | 1.00 | 106.99 |
| 2209 | CG | ARG | B | 393 | 29.251 | 53.196 | 37.704 | 1.00 | 109.77 |
| 2210 | CD | ARG | B | 393 | 28.521 | 52.107 | 38.464 | 1.00 | 115.73 |
| 2211 | NE | ARG | B | 393 | 27.095 | 52.085 | 38.165 | 1.00 | 119.73 |
| 2212 | CZ | ARG | B | 393 | 26.227 | 51.267 | 38.750 | 1.00 | 121.60 |
| 2213 | NH1 | ARG | B | 393 | 26.645 | 50.406 | 39.670 | 1.00 | 121.59 |
| 2214 | NH2 | ARG | B | 393 | 24.945 | 51.308 | 38.416 | 1.00 | 121.49 |
| 2215 | N | ASN | B | 394 | 30.942 | 55.391 | 35.224 | 1.00 | 103.98 |
| 2216 | CA | ASN | B | 394 | 30.630 | 55.595 | 33.819 | 1.00 | 106.60 |
| 2217 | C | ASN | B | 394 | 31.918 | 55.325 | 33.049 | 1.00 | 106.89 |
| 2218 | O | ASN | B | 394 | 31.950 | 54.516 | 32.122 | 1.00 | 108.55 |
| 2219 | CB | ASN | B | 394 | 30.173 | 57.036 | 33.580 | 1.00 | 108.97 |
| 2220 | CG | ASN | B | 394 | 29.709 | 57.274 | 32.153 | 1.00 | 112.83 |
| 2221 | OD1 | ASN | B | 394 | 29.459 | 58.410 | 31.752 | 1.00 | 114.53 |
| 2222 | ND2 | ASN | B | 394 | 29.584 | 56.198 | 31.381 | 1.00 | 114.40 |
| 2223 | N | GLY | B | 395 | 32.984 | 56.007 | 33.452 | 1.00 | 105.53 |
| 2224 | CA | GLY | B | 395 | 34.269 | 55.829 | 32.802 | 1.00 | 101.15 |
| 2225 | C | GLY | B | 395 | 35.177 | 57.015 | 33.050 | 1.00 | 97.82 |
| 2226 | O | GLY | B | 395 | 35.879 | 57.475 | 32.148 | 1.00 | 99.41 |
| 2227 | N | THR | B | 396 | 35.177 | 57.516 | 34.278 | 1.00 | 94.50 |
| 2228 | CA | THR | B | 396 | 36.011 | 58.663 | 34.578 | 1.00 | 89.02 |
| 2229 | C | THR | B | 396 | 36.426 | 58.837 | 36.029 | 1.00 | 83.93 |
| 2230 | O | THR | B | 396 | 35.600 | 59.123 | 36.896 | 1.00 | 83.27 |
| 2231 | CB | THR | B | 396 | 35.322 | 59.975 | 34.130 | 1.00 | 92.03 |
| 2232 | OG1 | THR | B | 396 | 35.116 | 59.952 | 32.712 | 1.00 | 94.17 |
| 2233 | CG2 | THR | B | 396 | 36.179 | 61.186 | 34.504 | 1.00 | 95.48 |
| 2234 | N | LEU | B | 397 | 37.718 | 58.658 | 36.279 | 1.00 | 79.69 |
| 2235 | CA | LEU | B | 397 | 38.280 | 58.871 | 37.600 | 1.00 | 74.75 |
| 2236 | C | LEU | B | 397 | 38.773 | 60.305 | 37.496 | 1.00 | 71.50 |
| 2237 | O | LEU | B | 397 | 39.880 | 60.548 | 37.019 | 1.00 | 74.81 |
| 2238 | CB | LEU | B | 397 | 39.480 | 57.960 | 37.860 | 1.00 | 75.72 |
| 2239 | CG | LEU | B | 397 | 40.350 | 58.412 | 39.047 | 1.00 | 70.67 |
| 2240 | CD1 | LEU | B | 397 | 39.742 | 57.932 | 40.362 | 1.00 | 69.05 |
| 2241 | CD2 | LEU | B | 397 | 41.768 | 57.877 | 38.887 | 1.00 | 72.96 |
| 2242 | N | THR | B | 398 | 37.941 | 61.252 | 37.905 | 1.00 | 67.61 |
| 2243 | CA | THR | B | 398 | 38.314 | 62.655 | 37.848 | 1.00 | 61.30 |
| 2244 | C | THR | B | 398 | 39.207 | 63.041 | 39.024 | 1.00 | 56.61 |
| 2245 | O | THR | B | 398 | 38.974 | 62.640 | 40.166 | 1.00 | 54.95 |
| 2246 | CB | THR | B | 398 | 37.070 | 63.556 | 37.852 | 1.00 | 61.75 |
| 2247 | OG1 | THR | B | 398 | 36.345 | 63.375 | 36.629 | 1.00 | 65.40 |
| 2248 | CG2 | THR | B | 398 | 37.471 | 65.016 | 37.992 | 1.00 | 63.39 |
| 2249 | N | VAL | B | 399 | 40.241 | 63.817 | 38.729 | 1.00 | 51.84 |
| 2250 | CA | VAL | B | 399 | 41.163 | 64.279 | 39.751 | 1.00 | 48.05 |
| 2251 | C | VAL | B | 399 | 41.515 | 65.730 | 39.490 | 1.00 | 44.81 |
| 2252 | O | VAL | B | 399 | 41.911 | 66.098 | 38.381 | 1.00 | 44.41 |
| 2253 | CB | VAL | B | 399 | 42.466 | 63.468 | 39.751 | 1.00 | 49.35 |
| 2254 | CB1 | VAL | B | 399 | 43.409 | 64.010 | 40.805 | 1.00 | 49.43 |
| 2255 | CG2 | VAL | B | 399 | 42.168 | 62.006 | 40.010 | 1.00 | 53.10 |
| 2256 | N | THR | B | 400 | 41.354 | 66.564 | 40.504 | 1.00 | 42.44 |
| 2257 | CA | THR | B | 400 | 41.691 | 67.965 | 40.343 | 1.00 | 40.44 |
| 2258 | C | THR | B | 400 | 42.532 | 68.449 | 41.498 | 1.00 | 38.07 |
| 2259 | O | THR | B | 400 | 42.483 | 67.908 | 42.602 | 1.00 | 36.13 |
| 2260 | CB | THR | B | 400 | 40.448 | 68.869 | 40.266 | 1.00 | 39.64 |
| 2261 | OG1 | THR | B | 400 | 39.845 | 68.962 | 41.561 | 1.00 | 43.72 |
| 2262 | CG2 | THR | B | 400 | 39.442 | 68.322 | 39.278 | 1.00 | 38.32 |
| 2263 | N | SER | B | 401 | 43.335 | 69.460 | 41.216 | 1.00 | 36.90 |
| 2264 | CA | SER | B | 401 | 44.168 | 70.069 | 42.224 | 1.00 | 35.46 |
| 2265 | C | SER | B | 401 | 43.988 | 71.557 | 42.016 | 1.00 | 33.95 |
| 2266 | O | SER | B | 401 | 44.153 | 72.062 | 40.908 | 1.00 | 34.80 |
| 2267 | CB | SER | B | 401 | 45.638 | 69.694 | 42.040 | 1.00 | 36.91 |
| 2268 | OG | SER | B | 401 | 46.417 | 70.301 | 43.060 | 1.00 | 40.84 |
| 2269 | N | THR | B | 402 | 43.635 | 72.248 | 43.086 | 1.00 | 32.28 |
| 2270 | CA | THR | B | 402 | 43.422 | 73.672 | 43.032 | 1.00 | 31.85 |
| 2271 | C | THR | B | 402 | 44.508 | 74.355 | 43.836 | 1.00 | 31.76 |
| 2272 | O | THR | B | 402 | 44.737 | 74.046 | 45.010 | 1.00 | 30.55 |
| 2273 | CB | THR | B | 402 | 42.051 | 74.030 | 43.595 | 1.00 | 31.33 |
| 2274 | OG1 | THR | B | 402 | 41.058 | 73.316 | 42.862 | 1.00 | 32.83 |
| 2275 | CG2 | THR | B | 402 | 41.782 | 75.522 | 43.468 | 1.00 | 32.70 |
| 2276 | N | LEU | B | 403 | 45.167 | 75.301 | 43.188 | 1.00 | 30.72 |
| 2277 | CA | LEU | B | 403 | 46.257 | 76.018 | 43.800 | 1.00 | 30.33 |
| 2278 | C | LEU | B | 403 | 45.927 | 77.485 | 44.003 | 1.00 | 29.66 |
| 2279 | O | LEU | B | 403 | 45.608 | 78.185 | 43.048 | 1.00 | 28.06 |
| 2280 | CB | LEU | B | 403 | 47.502 | 75.902 | 42.913 | 1.00 | 28.34 |
| 2281 | CG | LEU | B | 403 | 48.769 | 76.632 | 43.381 | 1.00 | 32.10 |
| 2282 | CD1 | LEU | B | 403 | 49.425 | 75.823 | 44.503 | 1.00 | 31.94 |
| 2283 | CD2 | LEU | B | 403 | 49.756 | 76.790 | 42.224 | 1.00 | 30.42 |
| 2284 | N | PRO | B | 404 | 45.983 | 77.962 | 45.259 | 1.00 | 31.48 |
| 2285 | CA | PRO | B | 404 | 45.704 | 79.367 | 45.566 | 1.00 | 33.20 |
| 2286 | C | PRO | B | 404 | 46.841 | 80.149 | 44.906 | 1.00 | 34.02 |
| 2287 | O | PRO | B | 404 | 47.999 | 79.755 | 44.996 | 1.00 | 32.25 |
| 2288 | CB | PRO | B | 404 | 45.788 | 79.417 | 47.090 | 1.00 | 33.28 |
| 2289 | CG | PRO | B | 404 | 45.435 | 78.010 | 47.500 | 1.00 | 31.82 |
| 2290 | CD | PRO | B | 404 | 46.186 | 77.187 | 46.497 | 1.00 | 31.26 |
| 2291 | N | VAL | B | 405 | 46.503 | 81.238 | 44.235 | 1.00 | 34.94 |
| 2292 | CA | VAL | B | 405 | 47.483 | 82.044 | 43.536 | 1.00 | 38.63 |
| 2293 | C | VAL | B | 405 | 47.541 | 83.472 | 44.068 | 1.00 | 40.02 |
| 2294 | O | VAL | B | 405 | 46.537 | 84.027 | 44.502 | 1.00 | 40.31 |
| 2295 | CB | VAL | B | 405 | 47.143 | 82.052 | 42.018 | 1.00 | 42.09 |
| 2296 | CG1 | VAL | B | 405 | 47.110 | 83.472 | 41.468 | 1.00 | 43.95 |
| 2297 | CG2 | VAL | B | 405 | 48.144 | 81.195 | 41.273 | 1.00 | 41.76 |
| 2298 | N | GLY | B | 406 | 48.728 | 84.064 | 44.039 | 1.00 | 42.35 |
| 2299 | CA | CLY | B | 406 | 48.859 | 85.432 | 44.503 | 1.00 | 43.35 |
| 2300 | C | GLY | B | 406 | 48.178 | 86.354 | 43.513 | 1.00 | 43.87 |
| 2301 | O | GLY | B | 406 | 48.286 | 86.154 | 42.303 | 1.00 | 42.68 |
| 2302 | N | PER | B | 407 | 47.466 | 87.352 | 44.023 | 1.00 | 44.40 |
| 2303 | CA | THR | B | 407 | 46.760 | 88.306 | 43.177 | 1.00 | 47.37 |
| 2304 | C | THR | B | 407 | 47.713 | 89.127 | 42.322 | 1.00 | 48.27 |
| 2305 | O | THR | B | 407 | 47.549 | 89.208 | 41.107 | 1.00 | 48.77 |
| 2306 | CB | THR | B | 407 | 45.922 | 89.288 | 44.022 | 1.00 | 48.44 |
| 2307 | OG1 | THR | B | 407 | 44.809 | 88.599 | 44.596 | 1.00 | 50.00 |
| 2308 | CG2 | THR | B | 407 | 45.413 | 90.436 | 43.162 | 1.00 | 53.23 |
| 2309 | N | ARG | B | 408 | 48.697 | 89.746 | 42.970 | 1.00 | 48.10 |
| 2310 | CA | ARG | B | 408 | 49.665 | 90.579 | 42.279 | 1.00 | 48.54 |
| 2311 | C | ARG | B | 408 | 50.540 | 89.773 | 41.335 | 1.00 | 48.48 |
| 2312 | O | ARG | B | 408 | 50.769 | 90.182 | 40.197 | 1.00 | 47.34 |
| 2313 | CB | ARG | B | 408 | 50.530 | 91.325 | 43.290 | 1.00 | 50.16 |
| 2314 | N | ASP | B | 409 | 51.033 | 88.634 | 41.806 | 1.00 | 48.72 |
| 2315 | CA | ASP | B | 409 | 51.886 | 87.787 | 40.979 | 1.00 | 50.88 |
| 2316 | C | ASP | B | 409 | 51.187 | 87.421 | 39.674 | 1.00 | 49.92 |
| 2317 | O | ASP | B | 409 | 51.789 | 87.468 | 38.598 | 1.00 | 49.76 |
| 2318 | CB | ASP | B | 409 | 52.260 | 86.512 | 41.738 | 1.00 | 56.18 |
| 2319 | CG | ASP | B | 409 | 53.013 | 86.801 | 43.026 | 1.00 | 64.80 |
| 2320 | OD1 | ASP | B | 409 | 54.062 | 87.482 | 42.956 | 1.00 | 71.41 |
| 2321 | OD2 | ASP | B | 409 | 52.561 | 86.353 | 44.105 | 1.00 | 68.29 |
| 2322 | N | THR | B | 410 | 49.908 | 87.067 | 39.773 | 1.00 | 48.36 |
| 2323 | CA | THR | B | 410 | 49.142 | 86.686 | 38.597 | 1.00 | 46.32 |
| 2324 | C | THR | B | 410 | 48.953 | 87.864 | 37.653 | 1.00 | 46.21 |
| 2325 | O | THR | B | 410 | 49.111 | 87.731 | 36.440 | 1.00 | 44.64 |
| 2326 | CB | THR | B | 410 | 47.774 | 86.120 | 38.992 | 1.00 | 42.82 |
| 2327 | CG | THR | B | 410 | 47.001 | 85.710 | 37.796 | 1.00 | 39.31 |
| 2328 | CD1 | THR | B | 410 | 46.104 | 86.467 | 37.103 | 1.00 | 37.29 |
| 2329 | CD2 | THR | B | 410 | 47.181 | 84.507 | 37.043 | 1.00 | 35.49 |
| 2330 | NH1 | THR | B | 410 | 45.726 | 85.819 | 35.957 | 1.00 | 39.24 |
| 2331 | CE2 | THR | B | 410 | 46.372 | 84.613 | 35.895 | 1.00 | 32.76 |
| 2332 | CE3 | THR | B | 410 | 47.956 | 83.351 | 37.228 | 1.00 | 32.07 |
| 2333 | CZ2 | THR | B | 410 | 46.309 | 83.606 | 34.924 | 1.00 | 29.59 |
| 2334 | CZ3 | THR | B | 410 | 47.896 | 82.352 | 36.269 | 1.00 | 33.68 |
| 2335 | CH2 | THR | B | 410 | 47.073 | 82.489 | 35.125 | 1.00 | 30.56 |
| 2336 | N | ILE | B | 411 | 48.611 | 89.016 | 38.212 | 1.00 | 48.11 |
| 2337 | CA | ILE | B | 411 | 48.408 | 90.209 | 37.401 | 1.00 | 50.90 |
| 2338 | C | ILE | B | 411 | 49.712 | 90.649 | 36.742 | 1.00 | 52.46 |
| 2339 | O | ILE | B | 411 | 49.698 | 91.229 | 35.658 | 1.00 | 53.34 |
| 2340 | CB | ILE | B | 411 | 47.845 | 91.366 | 38.246 | 1.00 | 51.58 |
| 2341 | CG1 | ILE | B | 411 | 46.456 | 90.987 | 38.770 | 1.00 | 52.81 |
| 2342 | CG2 | ILE | B | 411 | 47.772 | 92.634 | 37.415 | 1.00 | 51.95 |
| 2343 | CD1 | ILE | B | 411 | 45.823 | 92.034 | 39.669 | 1.00 | 59.38 |
| 2344 | N | GLU | B | 412 | 50.837 | 90.348 | 37.385 | 1.00 | 53.21 |
| 2345 | CA | GLU | B | 412 | 52.137 | 90.728 | 36.852 | 1.00 | 53.12 |
| 2346 | C | GLU | B | 412 | 52.681 | 89.747 | 35.831 | 1.00 | 51.24 |
| 2347 | O | GLU | B | 412 | 53.736 | 89.990 | 35.252 | 1.00 | 51.40 |
| 2348 | CB | GLU | B | 412 | 53.152 | 90.909 | 37.981 | 1.00 | 59.71 |
| 2349 | CG | GLU | B | 412 | 53.022 | 92.238 | 38.717 | 1.00 | 70.52 |
| 2350 | CD | GLU | B | 412 | 54.060 | 92.399 | 39.810 | 1.00 | 78.58 |
| 2351 | OE1 | GLU | B | 412 | 55.262 | 92.198 | 39.527 | 1.00 | 83.44 |
| 2352 | OE2 | GLU | B | 412 | 53.676 | 92.732 | 40.950 | 1.00 | 82.10 |
| 2353 | N | GLY | B | 413 | 51.982 | 88.634 | 35.623 | 1.00 | 48.47 |
| 2354 | CA | GLY | B | 413 | 52.427 | 87.677 | 34.626 | 1.00 | 44.62 |
| 2355 | C | GLY | B | 413 | 53.116 | 86.387 | 35.032 | 1.00 | 44.15 |
| 2356 | O | GLY | B | 413 | 53.687 | 85.718 | 34.173 | 1.00 | 43.50 |
| 2357 | N | GLU | B | 414 | 53.085 | 86.012 | 36.307 | 1.00 | 42.78 |
| 2358 | CA | GLU | B | 414 | 53.722 | 84.757 | 36.694 | 1.00 | 40.78 |
| 2359 | C | GLU | B | 414 | 53.105 | 83.652 | 35.851 | 1.00 | 39.97 |
| 2360 | O | GLU | B | 414 | 51.955 | 83.758 | 35.410 | 1.00 | 37.43 |
| 2361 | CB | GLU | B | 414 | 53.489 | 84.445 | 38.182 | 1.00 | 41.96 |
| 2362 | CG | GLU | B | 414 | 54.034 | 83.081 | 38.659 | 1.00 | 44.09 |
| 2363 | CD | GLU | B | 414 | 55.564 | 83.018 | 38.740 | 1.00 | 49.01 |
| 2364 | OE1 | GLU | B | 414 | 56.213 | 82.549 | 37.775 | 1.00 | 46.84 |
| 2365 | OE2 | GLU | B | 414 | 56.121 | 83.448 | 39.773 | 1.00 | 49.89 |
| 2366 | N | THR | B | 415 | 53.879 | 82.599 | 35.615 | 1.00 | 39.56 |
| 2367 | CA | THR | B | 415 | 53.403 | 81.464 | 34.847 | 1.00 | 40.76 |
| 2368 | C | THR | B | 415 | 53.487 | 80.231 | 35.738 | 1.00 | 40.07 |
| 2369 | O | THR | B | 415 | 54.533 | 79.960 | 36.323 | 1.00 | 42.10 |
| 2370 | CB | THR | B | 415 | 54.238 | 81.273 | 33.556 | 1.00 | 44.69 |
| 2371 | OG1 | THR | B | 415 | 54.222 | 79.893 | 33.171 | 1.00 | 51.98 |
| 2372 | CG2 | THR | B | 415 | 55.652 | 81.744 | 33.758 | 1.00 | 50.00 |
| 2373 | N | TYR | B | 416 | 52.380 | 79.500 | 35.860 | 1.00 | 37.57 |
| 2374 | CA | TYR | B | 416 | 52.327 | 78.318 | 36.714 | 1.00 | 34.50 |
| 2375 | C | TYR | B | 416 | 52.265 | 77.051 | 35.899 | 1.00 | 36.77 |
| 2376 | O | TYR | B | 416 | 51.706 | 77.038 | 34.800 | 1.00 | 35.94 |
| 2377 | CB | TYR | B | 416 | 51.116 | 78.379 | 37.634 | 1.00 | 34.15 |
| 2378 | CG | TYR | B | 416 | 51.085 | 79.597 | 38.514 | 1.00 | 30.65 |
| 2379 | CD1 | TYR | B | 416 | 50.777 | 80.856 | 37.992 | 1.00 | 32.53 |
| 2380 | CD2 | TYR | B | 416 | 51.389 | 79.499 | 39.865 | 1.00 | 31.72 |
| 2381 | CE1 | TYR | B | 416 | 50.772 | 81.984 | 38.803 | 1.00 | 34.27 |
| 2382 | CE2 | TYR | B | 416 | 51.390 | 80.619 | 40.680 | 1.00 | 32.11 |
| 2383 | CZ | TYR | B | 416 | 51.080 | 81.856 | 40.144 | 1.00 | 33.86 |
| 2384 | OH | TYR | B | 416 | 51.082 | 82.964 | 40.961 | 1.00 | 41.22 |
| 2385 | N | GLN | B | 417 | 52.829 | 75.976 | 36.441 | 1.00 | 37.58 |
| 2386 | CA | GLN | B | 417 | 52.848 | 74.722 | 35.714 | 1.00 | 39.43 |
| 2387 | C | GLN | B | 417 | 52.341 | 73.538 | 36.518 | 1.00 | 39.40 |
| 2388 | O | GLN | B | 417 | 52.664 | 73.373 | 37.694 | 1.00 | 39.63 |
| 2389 | CB | GLN | B | 417 | 54.269 | 74.421 | 35.211 | 1.00 | 41.37 |
| 2390 | CG | GLN | B | 417 | 54.350 | 73.214 | 34.278 | 1.00 | 51.81 |
| 2391 | CD | GLN | B | 417 | 55.773 | 72.856 | 33.886 | 1.00 | 61.69 |
| 2392 | OE1 | GLN | B | 417 | 56.555 | 72.371 | 34.710 | 1.00 | 67.15 |
| 2393 | NE2 | GLN | B | 417 | 56.119 | 73.096 | 32.621 | 1.00 | 63.64 |
| 2394 | N | CYS | B | 418 | 51.543 | 72.712 | 35.864 | 1.00 | 39.29 |
| 2395 | CA | CYS | B | 418 | 51.019 | 71.517 | 36.489 | 1.00 | 41.80 |
| 2396 | C | CYS | B | 418 | 51.720 | 70.350 | 35.818 | 1.00 | 42.50 |
| 2397 | O | CYS | B | 418 | 51.646 | 70.191 | 34.600 | 1.00 | 43.33 |
| 2398 | CB | CYS | B | 418 | 49.501 | 71.392 | 36.275 | 1.00 | 41.95 |
| 2399 | SG | CYS | B | 418 | 48.795 | 69.904 | 37.057 | 1.00 | 54.15 |
| 2400 | N | ARG | B | 419 | 52.418 | 69.551 | 36.609 | 1.00 | 43.98 |
| 2401 | CA | ARG | B | 419 | 53.103 | 68.375 | 36.093 | 1.00 | 47.51 |
| 2402 | C | ARG | B | 419 | 52.277 | 67.172 | 36.542 | 1.00 | 48.11 |
| 2403 | O | ARG | B | 419 | 52.219 | 66.859 | 37.730 | 1.00 | 47.85 |
| 2404 | CB | ARG | B | 419 | 54.532 | 68.311 | 36.648 | 1.00 | 49.08 |
| 2405 | CG | ARG | B | 419 | 55.203 | 66.958 | 36.514 | 1.00 | 54.11 |
| 2406 | CD | ARG | B | 419 | 56.708 | 67.046 | 36.796 | 1.00 | 64.71 |
| 2407 | NE | ARG | B | 419 | 57.325 | 65.732 | 36.963 | 1.00 | 64.09 |
| 2408 | CZ | ARG | B | 419 | 57.437 | 65.107 | 38.132 | 1.00 | 70.31 |
| 2409 | NH1 | ARG | B | 419 | 56.981 | 65.681 | 39.239 | 1.00 | 68.32 |
| 2410 | NH2 | ARG | B | 419 | 57.992 | 63.901 | 38.195 | 1.00 | 71.53 |
| 2411 | N | VAL | B | 420 | 51.610 | 66.524 | 35.592 | 1.00 | 51.04 |
| 2412 | CA | VAL | B | 420 | 50.776 | 65.369 | 35.900 | 1.00 | 56.22 |
| 2413 | C | VAL | B | 420 | 51.561 | 64.068 | 35.746 | 1.00 | 61.25 |
| 2414 | O | VAL | B | 420 | 52.178 | 63.820 | 34.707 | 1.00 | 60.11 |
| 2415 | CB | VAL | B | 420 | 49.536 | 65.305 | 34.985 | 1.00 | 54.44 |
| 2416 | CG1 | VAL | B | 420 | 48.678 | 64.106 | 35.371 | 1.00 | 52.69 |
| 2417 | CG2 | VAL | B | 420 | 48.732 | 66.597 | 35.095 | 1.00 | 55.45 |
| 2418 | N | THR | B | 421 | 51.529 | 63.241 | 36.786 | 1.00 | 66.23 |
| 2419 | CA | THR | B | 421 | 52.244 | 61.969 | 36.774 | 1.00 | 73.18 |
| 2420 | C | THR | B | 421 | 51.334 | 60.766 | 36.970 | 1.00 | 76.87 |
| 2421 | O | THR | B | 421 | 50.968 | 60.443 | 38.100 | 1.00 | 77.93 |
| 2422 | CB | THR | B | 421 | 53.317 | 61.929 | 37.875 | 1.00 | 73.75 |
| 2423 | OG1 | THR | B | 421 | 54.272 | 62.972 | 37.649 | 1.00 | 77.71 |
| 2424 | CG2 | THR | B | 421 | 54.033 | 60.591 | 37.873 | 1.00 | 75.35 |
| 2425 | N | HIS | B | 422 | 50.976 | 60.101 | 35.874 | 1.00 | 81.02 |
| 2426 | CA | HIS | B | 422 | 50.117 | 58.922 | 35.949 | 1.00 | 86.07 |
| 2427 | C | HIS | B | 422 | 51.004 | 57.686 | 35.822 | 1.00 | 89.67 |
| 2428 | O | HIS | B | 422 | 51.920 | 57.653 | 35.002 | 1.00 | 90.00 |
| 2429 | CB | HIS | B | 422 | 49.071 | 58.940 | 34.831 | 1.00 | 87.43 |
| 2430 | CG | HIS | B | 422 | 47.892 | 58.052 | 35.092 | 1.00 | 90.73 |
| 2431 | ND1 | HIS | B | 422 | 47.076 | 58.203 | 36.191 | 1.00 | 93.83 |
| 2432 | CD2 | HIS | B | 422 | 47.396 | 57.000 | 34.396 | 1.00 | 91.89 |
| 2433 | CE1 | HIS | B | 422 | 46.127 | 57.282 | 36.164 | 1.00 | 94.70 |
| 2434 | NE2 | HIS | B | 422 | 46.300 | 56.540 | 35.085 | 1.00 | 93.25 |
| 2435 | N | PRO | B | 423 | 50.729 | 56.646 | 36.626 | 1.00 | 92.64 |
| 2436 | CA | PRO | B | 423 | 51.489 | 55.391 | 36.640 | 1.00 | 95.19 |
| 2437 | C | PRO | B | 423 | 51.972 | 54.762 | 35.325 | 1.00 | 97.06 |
| 2438 | O | PRO | B | 423 | 53.169 | 54.536 | 35.155 | 1.00 | 97.57 |
| 2439 | CB | PRO | B | 423 | 50.576 | 54.445 | 37.433 | 1.00 | 95.56 |
| 2440 | CG | PRO | B | 423 | 49.209 | 55.034 | 37.247 | 1.00 | 93.94 |
| 2441 | CD | PRO | B | 423 | 49.488 | 56.495 | 37.404 | 1.00 | 93.12 |
| 2442 | N | HIS | B | 424 | 51.058 | 54.495 | 34.399 | 1.00 | 98.71 |
| 2443 | CA | HIS | B | 424 | 51.410 | 53.831 | 33.143 | 1.00 | 100.16 |
| 2444 | C | HIS | B | 424 | 52.104 | 54.579 | 32.005 | 1.00 | 99.67 |
| 2445 | O | HIS | B | 424 | 52.910 | 53.991 | 31.284 | 1.00 | 99.61 |
| 2446 | CB | HIS | B | 424 | 50.165 | 53.149 | 32.578 | 1.00 | 102.42 |
| 2447 | CG | HIS | B | 424 | 49.578 | 52.125 | 33.495 | 1.00 | 105.47 |
| 2448 | ND1 | HIS | B | 424 | 48.302 | 52.228 | 34.008 | 1.00 | 107.10 |
| 2449 | CD2 | HIS | B | 424 | 50.079 | 50.961 | 33.967 | 1.00 | 97.46 |
| 2450 | CE1 | HIS | B | 424 | 48.044 | 51.170 | 34.755 | 1.00 | 106.81 |
| 2451 | NE2 | HIS | B | 424 | 49.106 | 50.385 | 34.747 | 1.00 | 106.61 |
| 2452 | N | LEU | B | 425 | 51.802 | 55.858 | 31.833 | 1.00 | 99.19 |
| 2453 | CA | LEU | B | 425 | 52.384 | 56.628 | 30.737 | 1.00 | 99.01 |
| 2454 | C | LEU | B | 425 | 53.886 | 56.908 | 30.816 | 1.00 | 98.36 |
| 2455 | O | LEU | B | 425 | 54.440 | 57.121 | 31.896 | 1.00 | 98.39 |
| 2456 | CB | LEU | B | 425 | 51.606 | 57.929 | 30.593 | 1.00 | 100.66 |
| 2457 | CG | LEU | B | 425 | 50.105 | 57.631 | 30.633 | 1.00 | 101.95 |
| 2458 | CD1 | LEU | B | 425 | 49.315 | 58.917 | 30.531 | 1.00 | 102.59 |
| 2459 | CD2 | LEU | B | 425 | 49.744 | 56.677 | 29.502 | 1.00 | 103.85 |
| 2460 | N | PRO | B | 426 | 54.562 | 56.915 | 29.652 | 1.00 | 97.57 |
| 2461 | CA | PRO | B | 426 | 56.004 | 57.165 | 29.560 | 1.00 | 96.82 |
| 2462 | C | PRO | B | 426 | 56.414 | 58.525 | 30.108 | 1.00 | 95.53 |
| 2463 | O | PRO | B | 426 | 57.040 | 58.610 | 31.163 | 1.00 | 95.77 |
| 2464 | CB | PRO | B | 426 | 56.282 | 57.030 | 28.063 | 1.00 | 97.13 |
| 2465 | CG | PRO | B | 426 | 54.995 | 57.494 | 27.440 | 1.00 | 97.88 |
| 2466 | CD | PRO | B | 426 | 53.969 | 56.798 | 28.307 | 1.00 | 97.70 |
| 2467 | N | ARG | B | 427 | 56.064 | 59.585 | 29.387 | 1.00 | 94.16 |
| 2468 | CA | ARG | B | 427 | 56.402 | 60.931 | 29.826 | 1.00 | 92.52 |
| 2469 | C | ARG | B | 427 | 55.285 | 61.509 | 30.692 | 1.00 | 89.85 |
| 2470 | O | ARG | B | 427 | 54.123 | 61.113 | 30.583 | 1.00 | 89.26 |
| 2471 | CB | ARG | B | 427 | 56.652 | 61.851 | 28.622 | 1.00 | 97.34 |
| 2472 | CG | ARG | B | 427 | 57.202 | 63.219 | 29.023 | 1.00 | 99.32 |
| 2473 | CD | ARG | B | 427 | 57.407 | 64.172 | 27.847 | 1.00 | 105.99 |
| 2474 | NE | ARG | B | 427 | 58.068 | 65.403 | 28.285 | 1.00 | 111.10 |
| 2475 | CZ | ARG | B | 427 | 58.302 | 66.460 | 27.512 | 1.00 | 111.66 |
| 2476 | NH1 | ARG | B | 427 | 57.927 | 66.457 | 26.239 | 1.00 | 111.70 |
| 2477 | NH2 | ARG | B | 427 | 58.921 | 67.522 | 28.013 | 1.00 | 110.09 |
| 2478 | N | ALA | B | 428 | 55.651 | 62.442 | 31.562 | 1.00 | 86.56 |
| 2479 | CA | ALA | B | 428 | 54.690 | 63.087 | 32.439 | 1.00 | 82.70 |
| 2480 | C | ALA | B | 428 | 53.998 | 64.211 | 31.677 | 1.00 | 80.03 |
| 2481 | O | ALA | B | 428 | 54.619 | 64.889 | 30.861 | 1.00 | 80.29 |
| 2482 | CB | ALA | B | 428 | 55.400 | 63.647 | 33.663 | 1.00 | 83.85 |
| 2483 | N | LEU | B | 429 | 52.708 | 64.397 | 31.936 | 1.00 | 76.76 |
| 2484 | CA | LEU | B | 429 | 51.949 | 65.456 | 31.285 | 1.00 | 72.72 |
| 2485 | C | LEU | B | 429 | 52.311 | 66.780 | 31.936 | 1.00 | 69.67 |
| 2486 | O | LEU | B | 429 | 52.472 | 66.858 | 33.153 | 1.00 | 68.64 |
| 2487 | CB | LEU | B | 429 | 50.444 | 65.208 | 31.429 | 1.00 | 73.60 |
| 2488 | CG | LEU | B | 429 | 49.753 | 64.431 | 30.307 | 1.00 | 73.35 |
| 2489 | CD1 | LEU | B | 429 | 48.320 | 64.102 | 30.696 | 1.00 | 72.09 |
| 2490 | CD2 | LEU | B | 429 | 49.787 | 65.267 | 29.035 | 1.00 | 76.40 |
| 2491 | N | MET | B | 430 | 52.447 | 67.821 | 31.124 | 1.00 | 66.83 |
| 2492 | CA | MET | B | 430 | 52.786 | 69.132 | 31.651 | 1.00 | 64.81 |
| 2493 | C | MET | B | 430 | 51.922 | 70.216 | 31.026 | 1.00 | 61.41 |
| 2494 | O | MET | B | 430 | 51.824 | 70.326 | 29.801 | 1.00 | 60.84 |
| 2495 | CB | MET | B | 430 | 54.265 | 69.427 | 31.409 | 1.00 | 68.22 |
| 2496 | CG | MET | B | 430 | 55.184 | 68.353 | 31.967 | 1.00 | 74.39 |
| 2497 | SD | MET | B | 430 | 56.921 | 68.714 | 31.713 | 1.00 | 83.18 |
| 2498 | CE | MET | B | 430 | 56.993 | 68.764 | 29.906 | 1.00 | 84.12 |
| 2499 | N | ARG | B | 431 | 51.288 | 71.010 | 31.882 | 1.00 | 57.17 |
| 2500 | CA | ARG | B | 431 | 50.428 | 72.094 | 31.426 | 1.00 | 54.04 |
| 2501 | C | ARG | B | 431 | 50.848 | 73.366 | 32.137 | 1.00 | 50.58 |
| 2502 | O | ARG | B | 431 | 51.233 | 73.330 | 33.304 | 1.00 | 50.46 |
| 2503 | CB | ARG | B | 431 | 48.959 | 71.775 | 31.736 | 1.00 | 55.16 |
| 2504 | CG | ARG | B | 431 | 48.413 | 70.564 | 30.991 | 1.00 | 59.24 |
| 2505 | CD | ARG | B | 431 | 48.428 | 70.798 | 29.489 | 1.00 | 63.93 |
| 2506 | NE | ARG | B | 431 | 48.014 | 69.617 | 28.737 | 1.00 | 70.30 |
| 2507 | CZ | ARG | B | 431 | 48.043 | 69.530 | 27.411 | 1.00 | 76.24 |
| 2508 | NH1 | ARG | B | 431 | 48.469 | 70.559 | 26.683 | 1.00 | 77.83 |
| 2509 | NH2 | ARG | B | 431 | 47.648 | 68.415 | 26.811 | 1.00 | 77.12 |
| 2510 | N | SER | B | 432 | 50.788 | 74.488 | 31.431 | 1.00 | 46.92 |
| 2511 | CA | SER | B | 432 | 51.163 | 75.765 | 32.016 | 1.00 | 45.15 |
| 2512 | C | SER | B | 432 | 50.118 | 76.816 | 31.702 | 1.00 | 42.49 |
| 2513 | O | SER | B | 432 | 49.382 | 76.699 | 30.721 | 1.00 | 42.76 |
| 2514 | CB | SER | B | 432 | 52.532 | 76.206 | 31.490 | 1.00 | 44.31 |
| 2515 | OG | SER | B | 432 | 52.516 | 76.284 | 30.082 | 1.00 | 49.96 |
| 2516 | N | THR | B | 433 | 50.060 | 77.851 | 32.530 | 1.00 | 40.28 |
| 2517 | CA | THR | B | 433 | 49.078 | 78.909 | 32.337 | 1.00 | 39.71 |
| 2518 | C | THR | B | 433 | 49.604 | 80.244 | 32.843 | 1.00 | 39.49 |
| 2519 | O | THR | B | 433 | 50.414 | 80.285 | 33.766 | 1.00 | 40.22 |
| 2520 | CB | THR | B | 433 | 47.761 | 78.577 | 33.078 | 1.00 | 38.07 |
| 2521 | OG1 | THR | B | 433 | 46.793 | 79.594 | 32.805 | 1.00 | 39.56 |
| 2522 | CG2 | THR | B | 433 | 47.991 | 78.512 | 34.582 | 1.00 | 36.99 |
| 2523 | N | THR | B | 434 | 49.139 | 81.328 | 32.226 | 1.00 | 39.18 |
| 2524 | CA | THR | B | 434 | 49.532 | 82.687 | 32.588 | 1.00 | 40.70 |
| 2525 | C | THR | B | 434 | 48.414 | 83.635 | 32.166 | 1.00 | 40.23 |
| 2526 | O | THR | B | 434 | 47.509 | 83.236 | 31.441 | 1.00 | 41.18 |
| 2527 | CB | THR | B | 434 | 50.816 | 83.116 | 31.844 | 1.00 | 43.01 |
| 2528 | OG1 | THR | B | 434 | 50.548 | 83.238 | 30.439 | 1.00 | 47.85 |
| 2529 | CG2 | THR | B | 434 | 51.874 | 82.076 | 31.995 | 1.00 | 48.67 |
| 2530 | N | LYS | B | 435 | 48.475 | 84.886 | 32.604 | 1.00 | 40.97 |
| 2531 | CA | LYS | B | 435 | 47.450 | 85.833 | 32.211 | 1.00 | 45.73 |
| 2532 | C | LYS | B | 435 | 47.579 | 85.999 | 30.702 | 1.00 | 48.93 |
| 2533 | O | LYS | B | 435 | 48.670 | 85.890 | 30.158 | 1.00 | 48.49 |
| 2534 | CB | LYS | B | 435 | 47.663 | 87.166 | 32.906 | 1.00 | 45.94 |
| 2535 | N | THR | B | 436 | 46.467 | 86.235 | 30.018 | 1.00 | 52.01 |
| 2536 | CA | THR | B | 436 | 46.520 | 86.429 | 28.574 | 1.00 | 54.88 |
| 2537 | C | THR | B | 436 | 46.916 | 87.878 | 28.326 | 1.00 | 55.70 |
| 2538 | O | THR | B | 436 | 46.454 | 88.777 | 29.023 | 1.00 | 55.21 |
| 2539 | CB | THR | B | 436 | 45.146 | 86.162 | 27.909 | 1.00 | 56.24 |
| 2540 | OG1 | THR | B | 436 | 44.798 | 84.779 | 28.062 | 1.00 | 60.56 |
| 2541 | CG2 | THR | B | 436 | 45.188 | 86.510 | 26.423 | 1.00 | 57.04 |
| 2542 | N | SER | B | 437 | 47.790 | 88.099 | 27.352 | 1.00 | 57.93 |
| 2543 | CA | SER | B | 437 | 48.220 | 89.451 | 27.020 | 1.00 | 60.42 |
| 2544 | C | SER | B | 437 | 47.361 | 89.917 | 25.851 | 1.00 | 60.49 |
| 2545 | O | SER | B | 437 | 46.397 | 89.243 | 25.477 | 1.00 | 60.21 |
| 2546 | CB | SER | B | 437 | 49.706 | 89.467 | 26.630 | 1.00 | 61.57 |
| 2547 | OG | SER | B | 437 | 49.949 | 88.643 | 25.500 | 1.00 | 65.51 |
| 2548 | N | GLY | B | 438 | 47.705 | 91.067 | 25.279 | 1.00 | 60.09 |
| 2549 | CA | GLY | B | 438 | 46.934 | 91.578 | 24.161 | 1.00 | 58.42 |
| 2550 | C | GLY | B | 438 | 45.970 | 92.663 | 24.589 | 1.00 | 57.80 |
| 2551 | O | GLY | B | 438 | 45.853 | 92.952 | 25.778 | 1.00 | 56.51 |
| 2552 | N | PRO | B | 439 | 45.267 | 93.291 | 23.633 | 1.00 | 57.81 |
| 2553 | CA | PRO | B | 439 | 44.305 | 94.358 | 23.915 | 1.00 | 56.19 |
| 2554 | C | PRO | B | 439 | 43.178 | 93.908 | 24.837 | 1.00 | 53.91 |
| 2555 | O | PRO | B | 439 | 42.891 | 92.714 | 24.953 | 1.00 | 53.91 |
| 2556 | CB | PRO | B | 439 | 43.794 | 94.735 | 22.524 | 1.00 | 57.79 |
| 2557 | CG | PRO | B | 439 | 44.967 | 94.453 | 21.652 | 1.00 | 58.60 |
| 2558 | CD | PRO | B | 439 | 45.427 | 93.115 | 22.179 | 1.00 | 60.65 |
| 2559 | N | ARG | B | 440 | 42.545 | 94.878 | 25.488 | 1.00 | 51.09 |
| 2560 | CA | ARG | B | 440 | 41.437 | 94.610 | 26.398 | 1.00 | 48.90 |
| 2561 | C | ARG | B | 440 | 40.235 | 95.380 | 25.869 | 1.00 | 45.83 |
| 2562 | O | ARG | B | 440 | 40.398 | 96.412 | 25.219 | 1.00 | 45.88 |
| 2563 | CB | ARG | B | 440 | 41.764 | 95.099 | 27.811 | 1.00 | 49.95 |
| 2564 | CG | ARG | B | 440 | 43.127 | 94.681 | 28.329 | 1.00 | 59.03 |
| 2565 | CD | ARG | B | 440 | 43.227 | 93.194 | 28.652 | 1.00 | 65.31 |
| 2566 | NE | ARG | B | 440 | 44.627 | 92.786 | 28.758 | 1.00 | 72.31 |
| 2567 | CZ | ARG | B | 440 | 45.047 | 91.614 | 29.222 | 1.00 | 74.99 |
| 2568 | NH1 | ARG | B | 440 | 44.181 | 90.706 | 29.640 | 1.00 | 77.22 |
| 2569 | NH2 | ARG | B | 440 | 46.345 | 91.348 | 29.263 | 1.00 | 78.59 |
| 2570 | N | ALA | B | 441 | 39.036 | 94.873 | 26.143 | 1.00 | 42.09 |
| 2571 | CA | ALA | B | 441 | 37.800 | 95.521 | 25.713 | 1.00 | 39.26 |
| 2572 | C | ALA | B | 441 | 36.706 | 95.124 | 26.687 | 1.00 | 38.22 |
| 2573 | O | ALA | B | 441 | 36.566 | 93.951 | 27.024 | 1.00 | 38.95 |
| 2574 | CB | ALA | B | 441 | 37.430 | 95.089 | 24.302 | 1.00 | 36.56 |
| 2575 | N | ALA | B | 442 | 35.934 | 96.104 | 27.134 | 1.00 | 37.22 |
| 2576 | CA | ALA | B | 442 | 34.871 | 95.861 | 28.089 | 1.00 | 37.60 |
| 2577 | C | ALA | B | 442 | 33.721 | 95.050 | 27.510 | 1.00 | 38.65 |
| 2578 | O | ALA | B | 442 | 33.497 | 95.047 | 26.302 | 1.00 | 40.22 |
| 2579 | CB | ALA | B | 442 | 34.352 | 97.185 | 28.619 | 1.00 | 37.17 |
| 2580 | N | PRO | B | 443 | 32.981 | 94.340 | 28.378 | 1.00 | 38.04 |
| 2581 | CA | PRO | B | 443 | 31.835 | 93.511 | 27.996 | 1.00 | 36.79 |
| 2582 | C | PRO | B | 443 | 30.581 | 94.376 | 27.857 | 1.00 | 38.14 |
| 2583 | O | PRO | B | 443 | 30.426 | 95.364 | 28.579 | 1.00 | 37.39 |
| 2584 | CB | PRO | B | 443 | 31.677 | 92.550 | 29.179 | 1.00 | 36.30 |
| 2585 | CG | PRO | B | 443 | 32.968 | 92.654 | 29.941 | 1.00 | 36.35 |
| 2586 | CD | PRO | B | 443 | 33.351 | 94.088 | 29.780 | 1.00 | 37.85 |
| 2587 | N | GLU | B | 444 | 29.706 | 94.006 | 26.927 | 1.00 | 36.28 |
| 2588 | CA | GLU | B | 444 | 28.437 | 94.699 | 26.729 | 1.00 | 35.57 |
| 2589 | C | GLU | B | 444 | 27.429 | 93.659 | 27.212 | 1.00 | 33.87 |
| 2590 | O | GLU | B | 444 | 27.495 | 92.490 | 26.809 | 1.00 | 33.43 |
| 2591 | CB | GLU | B | 444 | 28.217 | 95.020 | 25.249 | 1.00 | 38.68 |
| 2592 | N | VAL | B | 445 | 26.503 | 94.080 | 28.067 | 1.00 | 30.18 |
| 2593 | CA | VAL | B | 445 | 25.530 | 93.168 | 28.650 | 1.00 | 28.24 |
| 2594 | C | VAL | B | 445 | 24.077 | 93.464 | 28.289 | 1.00 | 28.44 |
| 2595 | O | VAL | B | 445 | 23.625 | 94.600 | 28.379 | 1.00 | 26.78 |
| 2596 | CB | VAL | B | 445 | 25.663 | 93.183 | 30.195 | 1.00 | 28.10 |
| 2597 | CG1 | VAL | B | 445 | 24.667 | 92.230 | 30.826 | 1.00 | 25.56 |
| 2598 | CG2 | VAL | B | 445 | 27.079 | 92.829 | 30.589 | 1.00 | 26.30 |
| 2599 | N | TYR | B | 446 | 23.349 | 92.425 | 27.897 | 1.00 | 27.87 |
| 2600 | CA | TYR | B | 446 | 21.949 | 92.581 | 27.549 | 1.00 | 27.91 |
| 2601 | C | TYR | B | 446 | 21.140 | 91.407 | 28.076 | 1.00 | 27.06 |
| 2602 | O | TYR | B | 446 | 21.293 | 90.274 | 27.620 | 1.00 | 27.96 |
| 2603 | CB | TYR | B | 446 | 21.764 | 92.702 | 26.026 | 1.00 | 26.45 |
| 2604 | CG | TYR | B | 446 | 20.311 | 92.851 | 25.653 | 1.00 | 26.59 |
| 2605 | CD1 | TYR | B | 446 | 19.543 | 93.886 | 26.195 | 1.00 | 28.48 |
| 2606 | CD2 | TYR | B | 446 | 19.686 | 91.941 | 24.795 | 1.00 | 29.02 |
| 2607 | CE1 | TYR | B | 446 | 18.183 | 94.011 | 25.896 | 1.00 | 33.24 |
| 2608 | CE2 | TYR | B | 446 | 18.326 | 92.057 | 24.484 | 1.00 | 31.33 |
| 2609 | CZ | TYR | B | 446 | 17.585 | 93.094 | 25.041 | 1.00 | 36.01 |
| 2610 | OH | TYR | B | 446 | 16.248 | 93.213 | 24.762 | 1.00 | 36.54 |
| 2611 | N | ALA | B | 447 | 20.274 | 91.686 | 29.040 | 1.00 | 28.07 |
| 2612 | CA | ALA | B | 447 | 19.450 | 90.651 | 29.661 | 1.00 | 27.67 |
| 2613 | C | ALA | B | 447 | 18.021 | 90.785 | 29.168 | 1.00 | 29.19 |
| 2614 | O | ALA | B | 447 | 17.564 | 91.898 | 28.927 | 1.00 | 31.10 |
| 2615 | CB | ALA | B | 447 | 19.502 | 90.798 | 31.169 | 1.00 | 23.97 |
| 2616 | N | PHE | B | 448 | 17.307 | 89.671 | 29.021 | 1.00 | 29.56 |
| 2617 | CA | PHE | B | 448 | 15.943 | 89.751 | 28.522 | 1.00 | 32.50 |
| 2618 | C | PHE | B | 448 | 15.117 | 88.525 | 28.854 | 1.00 | 33.67 |
| 2619 | O | PHE | B | 448 | 15.650 | 87.488 | 29.228 | 1.00 | 35.26 |
| 2620 | CB | PHE | B | 448 | 15.970 | 89.941 | 27.003 | 1.00 | 35.29 |
| 2621 | CG | PHE | B | 448 | 16.523 | 88.751 | 26.262 | 1.00 | 37.04 |
| 2622 | CD1 | PHE | B | 448 | 15.691 | 87.688 | 25.905 | 1.00 | 36.66 |
| 2623 | CD2 | PHE | B | 448 | 17.886 | 88.666 | 25.973 | 1.00 | 34.95 |
| 2624 | CE1 | PHE | B | 448 | 16.218 | 86.551 | 25.276 | 1.00 | 40.91 |
| 2625 | CE2 | PHE | B | 448 | 18.422 | 87.534 | 25.347 | 1.00 | 35.99 |
| 2626 | CZ | PHE | B | 448 | 17.586 | 86.477 | 24.997 | 1.00 | 35.15 |
| 2627 | N | ALA | B | 449 | 13.804 | 88.648 | 28.704 | 1.00 | 35.81 |
| 2628 | CA | ALA | B | 449 | 12.907 | 87.534 | 28.969 | 1.00 | 37.63 |
| 2629 | C | ALA | B | 449 | 12.347 | 86.983 | 27.662 | 1.00 | 40.96 |
| 2630 | O | ALA | B | 449 | 12.159 | 87.715 | 26.697 | 1.00 | 40.14 |
| 2631 | CB | ALA | B | 449 | 11.769 | 87.977 | 29.870 | 1.00 | 33.60 |
| 2632 | N | THR | B | 450 | 12.091 | 85.682 | 27.652 | 1.00 | 45.40 |
| 2633 | CA | THR | B | 450 | 11.529 | 84.994 | 26.505 | 1.00 | 50.82 |
| 2634 | C | THR | B | 450 | 10.003 | 85.085 | 26.602 | 1.00 | 53.78 |
| 2635 | O | THR | B | 450 | 9.431 | 84.927 | 27.683 | 1.00 | 54.14 |
| 2636 | CB | THR | B | 450 | 11.935 | 83.510 | 26.513 | 1.00 | 51.45 |
| 2637 | OG1 | THR | B | 450 | 13.358 | 83.408 | 26.650 | 1.00 | 54.12 |
| 2638 | CG2 | THR | B | 450 | 11.507 | 82.831 | 25.223 | 1.00 | 55.58 |
| 2639 | N | PRO | B | 451 | 9.325 | 85.340 | 25.474 | 1.00 | 56.74 |
| 2640 | CA | PRO | B | 451 | 7.863 | 85.443 | 25.477 | 1.00 | 59.05 |
| 2641 | C | PRO | B | 451 | 7.209 | 84.173 | 26.020 | 1.00 | 61.00 |
| 2642 | O | PRO | B | 451 | 7.520 | 83.065 | 25.579 | 1.00 | 59.71 |
| 2643 | CB | PRO | B | 451 | 7.527 | 85.667 | 24.002 | 1.00 | 60.52 |
| 2644 | CG | PRO | B | 451 | 8.749 | 86.338 | 23.460 | 1.00 | 61.14 |
| 2645 | CD | PRO | B | 451 | 9.861 | 85.551 | 24.119 | 1.00 | 59.39 |
| 2646 | N | GLU | B | 452 | 6.315 | 84.328 | 26.988 | 1.00 | 64.30 |
| 2647 | CA | GLU | B | 452 | 5.625 | 83.172 | 27.542 | 1.00 | 69.17 |
| 2648 | C | GLU | B | 452 | 4.137 | 83.348 | 27.258 | 1.00 | 72.61 |
| 2649 | O | GLU | B | 452 | 3.394 | 83.933 | 28.043 | 1.00 | 73.59 |
| 2650 | CB | GLU | B | 452 | 5.888 | 83.040 | 29.046 | 1.00 | 65.66 |
| 2651 | CG | GLU | B | 452 | 6.034 | 81.585 | 29.523 | 1.00 | 61.46 |
| 2652 | CD | GLU | B | 452 | 7.283 | 80.883 | 28.967 | 1.00 | 60.52 |
| 2653 | OE1 | GLU | B | 452 | 8.336 | 81.547 | 28.849 | 1.00 | 63.47 |
| 2654 | OE2 | GLU | B | 452 | 7.227 | 79.666 | 28.667 | 1.00 | 53.34 |
| 2655 | N | THR | B | 453 | 3.731 | 82.837 | 26.103 | 1.00 | 76.75 |
| 2656 | CA | THR | B | 453 | 2.358 | 82.907 | 25.619 | 1.00 | 81.00 |
| 2657 | C | THR | B | 453 | 1.573 | 81.714 | 26.187 | 1.00 | 82.30 |
| 2658 | O | THR | B | 453 | 1.890 | 80.562 | 25.885 | 1.00 | 82.08 |
| 2659 | CB | THR | B | 453 | 2.409 | 82.863 | 24.090 | 1.00 | 85.65 |
| 2660 | CG | THR | B | 453 | 1.233 | 83.418 | 23.366 | 1.00 | 89.76 |
| 2661 | CD1 | THR | B | 453 | 0.679 | 84.661 | 23.512 | 1.00 | 92.34 |
| 2662 | CD2 | THR | B | 453 | 0.517 | 82.781 | 22.305 | 1.00 | 94.15 |
| 2663 | NH1 | THR | B | 453 | −0.334 | 84.836 | 22.598 | 1.00 | 94.99 |
| 2664 | CE2 | THR | B | 453 | −0.455 | 83.696 | 21.845 | 1.00 | 95.56 |
| 2665 | CE3 | THR | B | 453 | 0.605 | 81.521 | 21.693 | 1.00 | 95.36 |
| 2666 | CZ2 | THR | B | 453 | −1.335 | 83.392 | 20.800 | 1.00 | 97.14 |
| 2667 | CZ3 | THR | B | 453 | −0.269 | 81.220 | 20.655 | 1.00 | 95.61 |
| 2668 | CH2 | THR | B | 453 | −1.226 | 82.153 | 20.220 | 1.00 | 97.12 |
| 2669 | N | PRO | B | 454 | 0.542 | 81.981 | 27.020 | 1.00 | 84.10 |
| 2670 | CA | PRO | B | 454 | −0.331 | 80.994 | 27.678 | 1.00 | 87.24 |
| 2671 | C | PRO | B | 454 | −0.716 | 79.708 | 26.938 | 1.00 | 90.52 |
| 2672 | O | PRO | B | 454 | −0.378 | 79.512 | 25.767 | 1.00 | 89.75 |
| 2673 | CB | PRO | B | 454 | −1.550 | 81.825 | 28.112 | 1.00 | 84.91 |
| 2674 | CG | PRO | B | 454 | −1.417 | 83.125 | 27.360 | 1.00 | 83.62 |
| 2675 | CD | PRO | B | 454 | 0.063 | 83.341 | 27.307 | 1.00 | 84.60 |
| 2676 | N | GLY | B | 455 | −1.455 | 78.849 | 27.636 | 1.00 | 91.72 |
| 2677 | CA | GLY | B | 455 | −1.813 | 77.552 | 27.088 | 1.00 | 94.13 |
| 2678 | C | GLY | B | 455 | −0.532 | 76.870 | 27.499 | 1.00 | 96.28 |
| 2679 | O | CLY | B | 455 | 0.475 | 76.987 | 26.799 | 1.00 | 97.74 |
| 2680 | N | SER | B | 456 | −0.542 | 76.158 | 28.622 | 1.00 | 99.11 |
| 2681 | CA | SER | B | 456 | 0.731 | 75.626 | 29.077 | 1.00 | 96.15 |
| 2682 | C | SER | B | 456 | 1.135 | 74.193 | 29.424 | 1.00 | 91.50 |
| 2683 | O | SER | B | 456 | 0.316 | 73.308 | 29.767 | 1.00 | 88.52 |
| 2684 | CB | SER | B | 456 | 1.222 | 76.508 | 30.221 | 1.00 | 95.05 |
| 2685 | OG | SER | B | 456 | 0.225 | 76.690 | 31.216 | 1.00 | 110.76 |
| 2686 | N | ARG | B | 457 | 2.464 | 74.059 | 29.348 | 1.00 | 87.73 |
| 2687 | CA | ARG | B | 457 | 3.349 | 72.936 | 29.580 | 1.00 | 87.81 |
| 2688 | C | ARG | B | 457 | 4.497 | 73.922 | 29.453 | 1.00 | 86.30 |
| 2689 | O | ARG | B | 457 | 4.602 | 74.574 | 28.403 | 1.00 | 90.57 |
| 2690 | CB | ARG | B | 457 | 3.339 | 71.966 | 28.425 | 1.00 | 94.67 |
| 2691 | N | ASP | B | 458 | 5.341 | 74.088 | 30.473 | 1.00 | 87.32 |
| 2692 | CA | ASP | B | 458 | 6.314 | 75.159 | 30.320 | 1.00 | 80.43 |
| 2693 | C | ASP | B | 458 | 7.637 | 75.178 | 31.020 | 1.00 | 72.83 |
| 2694 | O | ASP | B | 458 | 8.163 | 74.190 | 31.501 | 1.00 | 73.96 |
| 2695 | CB | ASP | B | 458 | 5.615 | 76.450 | 30.715 | 1.00 | 83.19 |
| 2696 | CG | ASP | B | 458 | 5.300 | 76.498 | 32.210 | 1.00 | 85.09 |
| 2697 | OD1 | ASP | B | 458 | 4.694 | 75.535 | 32.722 | 1.00 | 91.25 |
| 2698 | OD2 | ASP | B | 458 | 5.661 | 77.492 | 32.883 | 1.00 | 90.84 |
| 2699 | N | LYS | B | 459 | 8.149 | 76.404 | 31.024 | 1.00 | 66.05 |
| 2700 | CA | LYS | B | 459 | 9.389 | 76.818 | 31.663 | 1.00 | 61.75 |
| 2701 | C | LYS | B | 459 | 9.617 | 78.232 | 31.214 | 1.00 | 56.14 |
| 2702 | O | LYS | B | 459 | 9.952 | 78.456 | 30.054 | 1.00 | 56.54 |
| 2703 | CB | LYS | B | 459 | 10.569 | 75.959 | 31.211 | 1.00 | 68.18 |
| 2704 | CG | LYS | B | 459 | 10.796 | 74.799 | 32.131 | 1.00 | 71.85 |
| 2705 | CD | LYS | B | 459 | 10.737 | 75.194 | 33.601 | 1.00 | 73.79 |
| 2706 | CE | LYS | B | 459 | 10.761 | 73.945 | 34.474 | 1.00 | 76.14 |
| 2707 | NZ | LYS | B | 459 | 10.925 | 74.259 | 35.931 | 1.00 | 79.35 |
| 2708 | N | ARG | B | 460 | 9.406 | 79.194 | 32.099 | 1.00 | 52.32 |
| 2709 | CA | ARG | B | 460 | 9.649 | 80.565 | 31.723 | 1.00 | 47.59 |
| 2710 | C | ARG | B | 460 | 11.164 | 80.740 | 31.810 | 1.00 | 43.34 |
| 2711 | O | ARG | B | 460 | 11.839 | 80.123 | 32.638 | 1.00 | 40.40 |
| 2712 | CB | ARG | B | 460 | 8.874 | 81.496 | 32.644 | 1.00 | 49.27 |
| 2713 | CG | ARG | B | 460 | 7.384 | 81.325 | 32.429 | 1.00 | 56.23 |
| 2714 | CD | ARG | B | 460 | 6.594 | 81.941 | 33.568 | 1.00 | 63.20 |
| 2715 | NE | ARG | B | 460 | 5.174 | 81.603 | 33.504 | 1.00 | 68.02 |
| 2716 | CZ | ARG | B | 460 | 4.645 | 80.455 | 33.920 | 1.00 | 70.46 |
| 2717 | NH1 | ARG | B | 460 | 5.415 | 79.506 | 34.444 | 1.00 | 72.23 |
| 2718 | NH2 | ARG | B | 460 | 3.336 | 80.264 | 33.815 | 1.00 | 73.53 |
| 2719 | N | THR | B | 461 | 11.690 | 81.538 | 30.902 | 1.00 | 39.19 |
| 2720 | CA | THR | B | 461 | 13.126 | 81.721 | 30.822 | 1.00 | 36.81 |
| 2721 | C | THR | B | 461 | 13.596 | 83.153 | 30.699 | 1.00 | 34.03 |
| 2722 | O | THR | B | 461 | 12.993 | 83.969 | 29.990 | 1.00 | 33.96 |
| 2723 | CB | THR | B | 461 | 13.710 | 80.997 | 29.588 | 1.00 | 39.13 |
| 2724 | OG1 | THR | B | 461 | 13.243 | 79.643 | 29.543 | 1.00 | 46.04 |
| 2725 | CG2 | THR | B | 461 | 15.220 | 80.970 | 29.679 | 1.00 | 45.48 |
| 2726 | N | LEU | B | 462 | 14.696 | 83.443 | 31.386 | 1.00 | 29.84 |
| 2727 | CA | LEU | B | 462 | 15.333 | 84.747 | 31.312 | 1.00 | 28.03 |
| 2728 | C | LEU | B | 462 | 16.674 | 84.395 | 30.690 | 1.00 | 27.33 |
| 2729 | O | LEU | B | 462 | 17.186 | 83.293 | 30.893 | 1.00 | 24.47 |
| 2730 | CB | LEU | B | 462 | 15.526 | 85.377 | 32.697 | 1.00 | 25.97 |
| 2731 | CG | LEU | B | 462 | 14.238 | 85.712 | 33.465 | 1.00 | 33.39 |
| 2732 | CD1 | LEU | B | 462 | 14.593 | 86.428 | 34.781 | 1.00 | 32.36 |
| 2733 | CD2 | LEU | B | 462 | 13.337 | 86.586 | 32.617 | 1.00 | 30.72 |
| 2734 | N | ALA | B | 463 | 17.241 | 85.314 | 29.922 | 1.00 | 25.83 |
| 2735 | CA | ALA | B | 463 | 18.489 | 85.023 | 29.264 | 1.00 | 25.65 |
| 2736 | C | ALA | B | 463 | 19.321 | 86.270 | 29.143 | 1.00 | 26.32 |
| 2737 | O | ALA | B | 463 | 18.810 | 87.386 | 29.254 | 1.00 | 27.53 |
| 2738 | CB | ALA | B | 463 | 18.219 | 84.421 | 27.888 | 1.00 | 28.15 |
| 2739 | N | CYS | B | 464 | 20.608 | 86.082 | 28.905 | 1.00 | 21.23 |
| 2740 | CA | CYS | B | 464 | 21.507 | 87.212 | 28.824 | 1.00 | 24.77 |
| 2741 | C | CYS | B | 464 | 22.584 | 87.001 | 27.786 | 1.00 | 22.14 |
| 2742 | O | CYS | B | 464 | 23.169 | 85.917 | 27.698 | 1.00 | 24.66 |
| 2743 | CB | CYS | B | 464 | 22.171 | 87.407 | 30.186 | 1.00 | 22.04 |
| 2744 | SG | CYS | B | 464 | 23.302 | 88.810 | 30.380 | 1.00 | 34.06 |
| 2745 | N | LEU | B | 465 | 22.859 | 88.052 | 27.025 | 1.00 | 22.44 |
| 2746 | CA | LEU | B | 465 | 23.903 | 88.002 | 26.011 | 1.00 | 23.11 |
| 2747 | C | LEU | B | 465 | 24.985 | 88.956 | 26.459 | 1.00 | 22.43 |
| 2748 | O | LEU | B | 465 | 24.702 | 90.117 | 26.748 | 1.00 | 23.73 |
| 2749 | CB | LEU | B | 465 | 23.370 | 88.445 | 24.639 | 1.00 | 22.99 |
| 2750 | CG | LEU | B | 465 | 24.423 | 88.790 | 23.562 | 1.00 | 19.04 |
| 2751 | CD1 | LEU | B | 465 | 25.269 | 87.565 | 23.234 | 1.00 | 21.72 |
| 2752 | CD2 | LEU | B | 465 | 23.716 | 89.253 | 22.282 | 1.00 | 28.23 |
| 2753 | N | ILE | B | 466 | 26.220 | 88.475 | 26.519 | 1.00 | 22.56 |
| 2754 | CA | ILE | B | 466 | 27.332 | 89.323 | 26.931 | 1.00 | 22.11 |
| 2755 | C | ILE | B | 466 | 28.344 | 89.298 | 25.803 | 1.00 | 24.25 |
| 2756 | O | ILE | B | 466 | 28.785 | 88.225 | 25.394 | 1.00 | 24.93 |
| 2757 | CB | ILE | B | 466 | 27.946 | 88.819 | 28.239 | 1.00 | 22.15 |
| 2758 | CG1 | ILE | B | 466 | 26.850 | 88.751 | 29.314 | 1.00 | 23.07 |
| 2759 | CG2 | ILE | B | 466 | 29.075 | 89.778 | 28.684 | 1.00 | 25.24 |
| 2760 | CD1 | ILE | B | 466 | 27.282 | 88.091 | 30.604 | 1.00 | 33.12 |
| 2761 | N | GLN | B | 467 | 28.737 | 90.468 | 25.307 | 1.00 | 24.63 |
| 2762 | CA | GLN | B | 467 | 29.630 | 90.480 | 24.149 | 1.00 | 28.64 |
| 2763 | C | GLN | B | 467 | 30.637 | 91.618 | 24.028 | 1.00 | 31.08 |
| 2764 | O | GLN | B | 467 | 30.685 | 92.529 | 24.865 | 1.00 | 31.00 |
| 2765 | CB | GLN | B | 467 | 28.770 | 90.428 | 22.872 | 1.00 | 25.58 |
| 2766 | CG | GLN | B | 467 | 27.873 | 91.657 | 22.696 | 1.00 | 25.53 |
| 2767 | CD | GLN | B | 467 | 26.907 | 91.556 | 21.501 | 1.00 | 30.72 |
| 2768 | OE1 | GLN | B | 467 | 27.144 | 90.810 | 20.551 | 1.00 | 29.76 |
| 2769 | NE2 | GLN | B | 467 | 25.821 | 92.327 | 21.550 | 1.00 | 32.18 |
| 2770 | N | ASN | B | 468 | 31.453 | 91.517 | 22.977 | 1.00 | 33.71 |
| 2771 | CA | ASN | B | 468 | 32.494 | 92.486 | 22.641 | 1.00 | 36.96 |
| 2772 | C | ASN | B | 468 | 33.601 | 92.607 | 23.677 | 1.00 | 38.20 |
| 2773 | O | ASN | B | 468 | 34.208 | 93.669 | 23.812 | 1.00 | 38.39 |
| 2774 | CB | ASN | B | 468 | 31.881 | 93.869 | 22.424 | 1.00 | 41.06 |
| 2775 | CG | ASN | B | 468 | 30.763 | 93.852 | 21.414 | 1.00 | 46.41 |
| 2776 | OD1 | ASN | B | 468 | 30.879 | 93.228 | 20.359 | 1.00 | 50.82 |
| 2777 | ND2 | ASN | B | 468 | 29.672 | 94.548 | 21.724 | 1.00 | 53.31 |
| 2778 | N | PHE | B | 469 | 33.868 | 91.535 | 24.415 | 1.00 | 35.74 |
| 2779 | CA | PHE | B | 469 | 34.910 | 91.597 | 25.423 | 1.00 | 33.87 |
| 2780 | C | PHE | B | 469 | 36.147 | 90.796 | 25.044 | 1.00 | 33.63 |
| 2781 | O | PHE | B | 469 | 36.087 | 89.873 | 24.240 | 1.00 | 34.78 |
| 2782 | CB | PHE | B | 469 | 34.372 | 91.105 | 26.778 | 1.00 | 29.12 |
| 2783 | CG | PHE | B | 469 | 33.909 | 89.674 | 26.766 | 1.00 | 26.27 |
| 2784 | CD1 | PHE | B | 469 | 34.808 | 88.641 | 26.967 | 1.00 | 22.45 |
| 2785 | CD2 | PHE | B | 469 | 32.566 | 89.362 | 26.561 | 1.00 | 22.06 |
| 2786 | CE1 | PHE | B | 469 | 34.386 | 87.315 | 26.974 | 1.00 | 23.84 |
| 2787 | CE2 | PHE | B | 469 | 32.135 | 88.036 | 26.564 | 1.00 | 28.02 |
| 2788 | CZ | PHE | B | 469 | 33.051 | 87.007 | 26.773 | 1.00 | 23.33 |
| 2789 | N | MET | B | 470 | 37.273 | 91.169 | 25.634 | 1.00 | 35.64 |
| 2790 | CA | MET | B | 470 | 38.535 | 90.480 | 25.413 | 1.00 | 37.62 |
| 2791 | C | MET | B | 470 | 39.496 | 90.916 | 26.504 | 1.00 | 36.36 |
| 2792 | O | MET | B | 470 | 39.478 | 92.071 | 26.923 | 1.00 | 36.33 |
| 2793 | CB | MET | B | 470 | 39.105 | 90.796 | 24.024 | 1.00 | 42.61 |
| 2794 | CG | MET | B | 470 | 39.421 | 92.246 | 23.750 | 1.00 | 50.60 |
| 2795 | SD | MET | B | 470 | 39.856 | 92.459 | 21.987 | 1.00 | 66.08 |
| 2796 | CE | MET | B | 470 | 41.544 | 91.820 | 21.966 | 1.00 | 66.35 |
| 2797 | N | PRO | B | 471 | 40.317 | 89.987 | 27.021 | 1.00 | 34.63 |
| 2798 | CA | PRO | B | 471 | 40.431 | 88.561 | 26.682 | 1.00 | 32.61 |
| 2799 | C | PRO | B | 471 | 39.147 | 87.782 | 26.957 | 1.00 | 31.92 |
| 2800 | O | PRO | B | 471 | 38.164 | 88.356 | 27.415 | 1.00 | 29.59 |
| 2801 | CB | PRO | B | 471 | 41.585 | 88.080 | 27.562 | 1.00 | 33.95 |
| 2802 | CG | PRO | B | 471 | 42.373 | 89.323 | 27.805 | 1.00 | 37.90 |
| 2803 | CD | PRO | B | 471 | 41.309 | 90.354 | 28.042 | 1.00 | 34.78 |
| 2804 | N | GLU | B | 472 | 39.187 | 86.472 | 26.714 | 1.00 | 32.28 |
| 2805 | CA | GLU | B | 472 | 38.020 | 85.599 | 26.866 | 1.00 | 36.49 |
| 2806 | C | GLU | B | 472 | 37.557 | 85.220 | 28.270 | 1.00 | 35.52 |
| 2807 | O | GLU | B | 472 | 36.438 | 84.730 | 28.417 | 1.00 | 34.87 |
| 2808 | CB | GLU | B | 472 | 38.223 | 84.298 | 26.085 | 1.00 | 41.97 |
| 2809 | CG | GLU | B | 472 | 39.294 | 83.387 | 26.677 | 1.00 | 54.78 |
| 2810 | CD | GLU | B | 472 | 39.456 | 82.073 | 25.916 | 1.00 | 62.41 |
| 2811 | OE1 | GLU | B | 472 | 39.021 | 81.997 | 24.744 | 1.00 | 67.26 |
| 2812 | OE2 | GLU | B | 472 | 40.034 | 81.119 | 26.487 | 1.00 | 64.96 |
| 2813 | N | ASP | B | 473 | 38.379 | 85.439 | 29.295 | 1.00 | 34.38 |
| 2814 | CA | ASP | B | 473 | 37.971 | 85.057 | 30.648 | 1.00 | 31.83 |
| 2815 | C | ASP | B | 473 | 36.914 | 85.998 | 31.214 | 1.00 | 31.24 |
| 2816 | O | ASP | B | 473 | 37.072 | 87.220 | 31.199 | 1.00 | 31.80 |
| 2817 | CB | ASP | B | 473 | 39.180 | 84.979 | 31.584 | 1.00 | 34.73 |
| 2818 | CG | ASP | B | 473 | 40.147 | 83.854 | 31.196 | 1.00 | 40.83 |
| 2819 | OD1 | ASP | B | 473 | 39.676 | 82.759 | 30.804 | 1.00 | 44.18 |
| 2820 | OD2 | ASP | B | 473 | 41.378 | 84.061 | 31.290 | 1.00 | 40.17 |
| 2821 | N | ILE | B | 474 | 35.826 | 85.416 | 31.702 | 1.00 | 26.96 |
| 2822 | CA | ILE | B | 474 | 34.744 | 86.207 | 32.246 | 1.00 | 25.39 |
| 2823 | C | ILE | B | 474 | 33.885 | 85.375 | 33.208 | 1.00 | 24.74 |
| 2824 | O | ILE | B | 474 | 33.776 | 84.164 | 33.069 | 1.00 | 25.20 |
| 2825 | CB | ILE | B | 474 | 33.862 | 86.758 | 31.092 | 1.00 | 23.81 |
| 2826 | CG1 | ILE | B | 474 | 32.925 | 87.852 | 31.598 | 1.00 | 22.23 |
| 2827 | CG2 | ILE | B | 474 | 33.062 | 85.626 | 30.453 | 1.00 | 27.10 |
| 2828 | CD1 | ILE | B | 474 | 32.144 | 88.536 | 30.450 | 1.00 | 25.30 |
| 2829 | N | SER | B | 475 | 33.312 | 86.030 | 34.209 | 1.00 | 23.27 |
| 2830 | CA | SER | B | 475 | 32.430 | 85.348 | 35.148 | 1.00 | 22.17 |
| 2831 | C | SER | B | 475 | 31.066 | 85.991 | 34.952 | 1.00 | 21.71 |
| 2832 | O | SER | B | 475 | 30.951 | 87.207 | 34.876 | 1.00 | 20.84 |
| 2833 | CB | SER | B | 475 | 32.879 | 85.551 | 36.593 | 1.00 | 20.43 |
| 2834 | OG | SER | B | 475 | 34.164 | 85.011 | 36.806 | 1.00 | 30.00 |
| 2835 | N | VAL | B | 476 | 30.042 | 85.158 | 34.887 | 1.00 | 21.76 |
| 2836 | CA | VAL | B | 476 | 28.694 | 85.620 | 34.675 | 1.00 | 21.72 |
| 2837 | C | VAL | B | 476 | 27.875 | 85.165 | 35.851 | 1.00 | 23.56 |
| 2838 | O | VAL | B | 476 | 28.036 | 84.045 | 36.320 | 1.00 | 25.95 |
| 2839 | CB | VAL | B | 476 | 28.087 | 84.994 | 33.385 | 1.00 | 21.56 |
| 2840 | CG1 | VAL | B | 476 | 26.625 | 85.443 | 33.222 | 1.00 | 18.70 |
| 2841 | CG2 | VAL | B | 476 | 28.915 | 85.403 | 32.166 | 1.00 | 20.99 |
| 2842 | N | GLN | B | 477 | 26.994 | 86.026 | 36.336 | 1.00 | 25.16 |
| 2843 | CA | GLN | B | 477 | 26.172 | 85.635 | 37.460 | 1.00 | 26.65 |
| 2844 | C | GLN | B | 477 | 24.833 | 86.337 | 37.438 | 1.00 | 26.62 |
| 2845 | O | GLN | B | 477 | 24.709 | 87.440 | 36.925 | 1.00 | 26.40 |
| 2846 | CB | GLN | B | 477 | 26.917 | 85.899 | 38.775 | 1.00 | 31.51 |
| 2847 | CG | GLN | B | 477 | 27.348 | 87.311 | 39.011 | 1.00 | 44.27 |
| 2848 | CD | GLN | B | 477 | 28.505 | 87.412 | 40.016 | 1.00 | 47.76 |
| 2849 | OE1 | GLN | B | 477 | 28.814 | 88.496 | 40.502 | 1.00 | 50.57 |
| 2850 | NE2 | GLN | B | 477 | 29.151 | 86.283 | 40.313 | 1.00 | 47.32 |
| 2851 | N | THR | B | 478 | 23.820 | 85.659 | 37.954 | 1.00 | 27.41 |
| 2852 | CA | THR | B | 478 | 22.488 | 86.225 | 38.026 | 1.00 | 28.36 |
| 2853 | C | THR | B | 478 | 22.192 | 86.656 | 39.465 | 1.00 | 30.10 |
| 2854 | O | THR | B | 478 | 22.660 | 86.033 | 40.428 | 1.00 | 25.31 |
| 2855 | CB | THR | B | 478 | 21.461 | 85.196 | 37.567 | 1.00 | 28.76 |
| 2856 | CG | THR | B | 478 | 21.502 | 84.951 | 36.087 | 1.00 | 34.11 |
| 2857 | CD1 | THR | B | 478 | 22.313 | 84.078 | 35.415 | 1.00 | 31.25 |
| 2858 | CD2 | THR | B | 478 | 20.722 | 85.620 | 35.094 | 1.00 | 31.01 |
| 2859 | NE1 | THR | B | 478 | 22.085 | 84.164 | 34.069 | 1.00 | 27.71 |
| 2860 | CE2 | THR | B | 478 | 21.113 | 85.104 | 33.841 | 1.00 | 31.95 |
| 2861 | CE3 | THR | B | 478 | 19.729 | 86.607 | 35.140 | 1.00 | 34.16 |
| 2862 | CZ2 | THR | B | 478 | 20.542 | 85.545 | 32.637 | 1.00 | 25.26 |
| 2863 | CZ3 | THR | B | 478 | 19.161 | 87.044 | 33.947 | 1.00 | 31.64 |
| 2864 | CH2 | THR | B | 478 | 19.571 | 86.511 | 32.713 | 1.00 | 28.89 |
| 2865 | N | LEU | B | 479 | 21.418 | 87.723 | 39.602 | 1.00 | 32.48 |
| 2866 | CA | LEU | B | 479 | 21.049 | 88.245 | 40.913 | 1.00 | 37.36 |
| 2867 | C | LEU | B | 479 | 19.584 | 88.657 | 40.960 | 1.00 | 40.42 |
| 2868 | O | LEU | B | 479 | 19.040 | 89.152 | 39.977 | 1.00 | 40.14 |
| 2869 | CB | LEU | B | 479 | 21.878 | 89.478 | 41.252 | 1.00 | 39.83 |
| 2870 | CG | LEU | B | 479 | 23.346 | 89.556 | 40.842 | 1.00 | 44.57 |
| 2871 | CD1 | LEU | B | 479 | 23.890 | 90.887 | 41.290 | 1.00 | 52.98 |
| 2872 | CD2 | LEU | B | 479 | 24.130 | 88.439 | 41.466 | 1.00 | 50.71 |
| 2873 | N | HIS | B | 480 | 18.947 | 88.448 | 42.102 | 1.00 | 43.05 |
| 2874 | CA | HIS | B | 480 | 17.572 | 88.877 | 42.276 | 1.00 | 49.20 |
| 2875 | C | HIS | B | 480 | 17.746 | 90.136 | 43.116 | 1.00 | 52.73 |
| 2876 | O | HIS | B | 480 | 17.705 | 90.091 | 44.347 | 1.00 | 52.90 |
| 2877 | CB | HIS | B | 480 | 16.754 | 87.844 | 43.043 | 1.00 | 52.25 |
| 2878 | CG | HIS | B | 480 | 15.341 | 88.270 | 43.286 | 1.00 | 57.22 |
| 2879 | ND1 | HIS | B | 480 | 14.337 | 88.075 | 42.364 | 1.00 | 61.37 |
| 2880 | CD2 | HIS | B | 480 | 14.778 | 88.942 | 44.319 | 1.00 | 59.16 |
| 2881 | CE1 | HIS | B | 480 | 13.216 | 88.607 | 42.815 | 1.00 | 59.88 |
| 2882 | NE2 | HIS | B | 480 | 13.456 | 89.140 | 44.000 | 1.00 | 66.11 |
| 2883 | N | ASN | B | 481 | 17.971 | 91.253 | 42.430 | 1.00 | 56.65 |
| 2884 | CA | ASN | B | 481 | 18.206 | 92.543 | 43.070 | 1.00 | 59.93 |
| 2885 | C | ASN | B | 481 | 19.660 | 92.591 | 43.519 | 1.00 | 59.65 |
| 2886 | O | ASN | B | 481 | 20.571 | 92.569 | 42.695 | 1.00 | 61.04 |
| 2887 | CB | ASN | B | 481 | 17.288 | 92.751 | 44.282 | 1.00 | 64.65 |
| 2888 | CG | ASN | B | 481 | 15.852 | 93.022 | 43.890 | 1.00 | 72.38 |
| 2889 | OG1 | ASN | B | 481 | 15.563 | 93.964 | 43.147 | 1.00 | 74.96 |
| 2890 | ND2 | ASN | B | 481 | 14.938 | 92.199 | 44.393 | 1.00 | 75.60 |
| 2891 | N | GLU | B | 482 | 19.872 | 92.625 | 44.827 | 1.00 | 58.97 |
| 2892 | CA | GLU | B | 482 | 21.213 | 92.697 | 45.396 | 1.00 | 58.55 |
| 2893 | C | GLU | B | 482 | 21.815 | 91.346 | 45.782 | 1.00 | 55.30 |
| 2894 | O | GLU | B | 482 | 23.013 | 91.253 | 46.044 | 1.00 | 57.27 |
| 2895 | CB | GLU | B | 482 | 21.176 | 93.607 | 46.627 | 1.00 | 65.42 |
| 2896 | CG | GLU | B | 482 | 19.760 | 93.813 | 47.172 | 1.00 | 73.94 |
| 2897 | CD | GLU | B | 482 | 19.735 | 94.473 | 48.532 | 1.00 | 80.29 |
| 2898 | OE1 | GLU | B | 482 | 20.168 | 93.830 | 49.513 | 1.00 | 86.31 |
| 2899 | OE2 | GLU | B | 482 | 19.284 | 95.633 | 48.620 | 1.00 | 82.73 |
| 2900 | N | VAL | B | 483 | 20.997 | 90.301 | 45.814 | 1.00 | 50.25 |
| 2901 | CA | VAL | B | 483 | 21.487 | 88.988 | 46.211 | 1.00 | 45.78 |
| 2902 | C | VAL | B | 483 | 21.733 | 88.025 | 45.052 | 1.00 | 43.04 |
| 2903 | O | VAL | B | 483 | 20.880 | 87.834 | 44.198 | 1.00 | 41.34 |
| 2904 | CB | VAL | B | 483 | 20.527 | 88.339 | 47.233 | 1.00 | 43.76 |
| 2905 | CG1 | VAL | B | 483 | 19.129 | 88.283 | 46.668 | 1.00 | 46.25 |
| 2906 | CG2 | VAL | B | 483 | 21.011 | 86.946 | 47.594 | 1.00 | 41.37 |
| 2907 | N | GLN | B | 484 | 22.912 | 87.412 | 45.050 | 1.00 | 42.30 |
| 2908 | CA | GLN | B | 484 | 23.300 | 86.474 | 44.005 | 1.00 | 43.21 |
| 2909 | C | GLN | B | 484 | 22.658 | 85.091 | 44.141 | 1.00 | 42.48 |
| 2910 | O | GLN | B | 484 | 22.538 | 84.545 | 45.244 | 1.00 | 41.64 |
| 2911 | CB | GLN | B | 484 | 24.826 | 86.331 | 43.971 | 1.00 | 45.96 |
| 2912 | CG | GLN | B | 484 | 25.324 | 85.269 | 42.999 | 1.00 | 54.43 |
| 2913 | CD | GLN | B | 484 | 26.840 | 85.173 | 42.941 | 1.00 | 59.02 |
| 2914 | OE1 | GLN | B | 484 | 27.391 | 84.200 | 42.423 | 1.00 | 59.71 |
| 2915 | NE2 | GLN | B | 484 | 27.522 | 86.190 | 43.464 | 1.00 | 61.46 |
| 2916 | N | LEU | B | 485 | 22.239 | 84.539 | 43.006 | 1.00 | 38.99 |
| 2917 | CA | LEU | B | 485 | 21.618 | 83.218 | 42.953 | 1.00 | 39.17 |
| 2918 | C | LEU | B | 485 | 22.700 | 82.142 | 42.853 | 1.00 | 40.35 |
| 2919 | O | LEU | B | 485 | 23.822 | 82.417 | 42.430 | 1.00 | 37.39 |
| 2920 | CB | LEU | B | 485 | 20.705 | 83.107 | 41.725 | 1.00 | 37.16 |
| 2921 | CG | LEU | B | 485 | 19.579 | 84.140 | 41.602 | 1.00 | 39.66 |
| 2922 | CD1 | LEU | B | 485 | 18.836 | 83.930 | 40.293 | 1.00 | 39.30 |
| 2923 | CD2 | LEU | B | 485 | 18.644 | 84.020 | 42.795 | 1.00 | 40.56 |
| 2924 | N | PRO | B | 486 | 22.373 | 80.900 | 43.242 | 1.00 | 42.55 |
| 2925 | CA | PRO | B | 486 | 23.366 | 79.828 | 43.158 | 1.00 | 46.30 |
| 2926 | C | PRO | B | 486 | 23.750 | 79.660 | 41.688 | 1.00 | 49.32 |
| 2927 | O | PRO | B | 486 | 22.916 | 79.852 | 40.806 | 1.00 | 48.81 |
| 2928 | CB | PRO | B | 486 | 22.606 | 78.609 | 43.675 | 1.00 | 45.64 |
| 2929 | CG | PRO | B | 486 | 21.532 | 79.187 | 44.528 | 1.00 | 45.29 |
| 2930 | CD | PRO | B | 486 | 21.095 | 80.394 | 43.769 | 1.00 | 40.65 |
| 2931 | N | ASP | B | 487 | 24.997 | 79.290 | 41.429 | 1.00 | 53.39 |
| 2932 | CA | ASP | B | 487 | 25.465 | 79.093 | 40.062 | 1.00 | 57.75 |
| 2933 | C | ASP | B | 487 | 24.660 | 77.997 | 39.350 | 1.00 | 57.25 |
| 2934 | O | ASP | B | 487 | 24.205 | 78.176 | 38.213 | 1.00 | 57.75 |
| 2935 | CB | ASP | B | 487 | 26.944 | 78.708 | 40.077 | 1.00 | 67.82 |
| 2936 | CG | ASP | B | 487 | 27.577 | 78.779 | 38.703 | 1.00 | 76.17 |
| 2937 | OD1 | ASP | B | 487 | 26.945 | 78.309 | 37.734 | 1.00 | 84.77 |
| 2938 | OD2 | ASP | B | 487 | 28.711 | 79.297 | 38.592 | 1.00 | 86.94 |
| 2939 | N | ALA | B | 488 | 24.478 | 76.873 | 40.035 | 1.00 | 54.64 |
| 2940 | CA | ALA | B | 488 | 23.752 | 75.729 | 39.493 | 1.00 | 53.45 |
| 2941 | C | ALA | B | 488 | 22.421 | 76.069 | 38.837 | 1.00 | 52.74 |
| 2942 | O | ALA | B | 488 | 21.843 | 75.241 | 38.134 | 1.00 | 53.06 |
| 2943 | CB | ALA | B | 488 | 23.530 | 74.708 | 40.585 | 1.00 | 53.75 |
| 2944 | N | ARG | B | 489 | 21.938 | 77.287 | 39.056 | 1.00 | 50.96 |
| 2945 | CA | ARG | B | 489 | 20.660 | 77.704 | 38.491 | 1.00 | 47.84 |
| 2946 | C | ARG | B | 489 | 20.705 | 78.164 | 37.036 | 1.00 | 45.10 |
| 2947 | O | ARG | B | 489 | 19.681 | 78.158 | 36.363 | 1.00 | 43.37 |
| 2948 | CB | ARG | B | 489 | 20.046 | 78.808 | 39.354 | 1.00 | 48.99 |
| 2949 | CG | ARG | B | 489 | 19.232 | 78.294 | 40.524 | 1.00 | 50.01 |
| 2950 | CD | ARG | B | 489 | 17.761 | 78.427 | 40.227 | 1.00 | 50.36 |
| 2951 | NE | ARG | B | 489 | 17.187 | 79.581 | 40.905 | 1.00 | 56.65 |
| 2952 | CZ | ARG | B | 489 | 16.048 | 80.166 | 40.557 | 1.00 | 58.23 |
| 2953 | NH1 | ARG | B | 489 | 15.352 | 79.714 | 39.522 | 1.00 | 61.07 |
| 2954 | NH2 | ARG | B | 489 | 15.594 | 81.193 | 41.260 | 1.00 | 59.63 |
| 2955 | N | HIS | B | 490 | 21.872 | 78.573 | 36.551 | 1.00 | 42.41 |
| 2956 | CA | HIS | B | 490 | 21.965 | 79.024 | 35.166 | 1.00 | 41.63 |
| 2957 | C | HIS | B | 490 | 22.952 | 78.210 | 34.334 | 1.00 | 39.71 |
| 2958 | O | HIS | B | 490 | 23.768 | 77.471 | 34.865 | 1.00 | 41.30 |
| 2959 | CB | HIS | B | 490 | 22.348 | 80.508 | 35.100 | 1.00 | 42.40 |
| 2960 | CG | HIS | B | 490 | 23.720 | 80.807 | 35.614 | 1.00 | 43.27 |
| 2961 | ND1 | HIS | B | 490 | 23.966 | 81.177 | 36.919 | 1.00 | 42.27 |
| 2962 | CD2 | HIS | B | 490 | 24.926 | 80.779 | 34.999 | 1.00 | 44.41 |
| 2963 | CE1 | HIS | B | 490 | 25.262 | 81.364 | 37.086 | 1.00 | 39.88 |
| 2964 | NE2 | HIS | B | 490 | 25.868 | 81.130 | 35.934 | 1.00 | 45.37 |
| 2965 | N | SER | B | 491 | 22.858 | 78.355 | 33.020 | 1.00 | 37.20 |
| 2966 | CA | SER | B | 491 | 23.734 | 77.654 | 32.095 | 1.00 | 36.36 |
| 2967 | C | SER | B | 491 | 24.401 | 78.685 | 31.173 | 1.00 | 33.06 |
| 2968 | O | SER | B | 491 | 23.721 | 79.428 | 30.461 | 1.00 | 33.77 |
| 2969 | CB | SER | B | 491 | 22.910 | 76.651 | 31.288 | 1.00 | 35.42 |
| 2970 | OG | SER | B | 491 | 23.694 | 76.053 | 30.282 | 1.00 | 43.65 |
| 2971 | N | THR | B | 492 | 25.728 | 78.722 | 31.188 | 1.00 | 29.90 |
| 2972 | CA | THR | B | 492 | 26.495 | 79.673 | 30.383 | 1.00 | 29.91 |
| 2973 | C | THR | B | 492 | 27.322 | 78.977 | 29.287 | 1.00 | 29.20 |
| 2974 | O | THR | B | 492 | 27.996 | 77.991 | 29.552 | 1.00 | 30.85 |
| 2975 | CB | THR | B | 492 | 27.447 | 80.506 | 31.298 | 1.00 | 29.85 |
| 2976 | OG1 | THR | B | 492 | 26.670 | 81.197 | 32.281 | 1.00 | 35.86 |
| 2977 | CG2 | THR | B | 492 | 28.234 | 81.541 | 30.498 | 1.00 | 28.05 |
| 2978 | N | THR | B | 493 | 27.281 | 79.505 | 28.065 | 1.00 | 29.49 |
| 2979 | CA | THR | B | 493 | 28.032 | 78.913 | 26.957 | 1.00 | 30.19 |
| 2980 | C | THR | B | 493 | 29.501 | 79.295 | 27.056 | 1.00 | 32.39 |
| 2981 | O | THR | B | 493 | 29.863 | 80.210 | 27.786 | 1.00 | 30.70 |
| 2982 | CB | THR | B | 493 | 27.502 | 79.378 | 25.568 | 1.00 | 27.82 |
| 2983 | OG1 | THR | B | 493 | 27.592 | 80.806 | 25.460 | 1.00 | 30.48 |
| 2984 | CG2 | THR | B | 493 | 26.067 | 78.953 | 25.372 | 1.00 | 22.03 |
| 2985 | N | GLN | B | 494 | 30.347 | 78.586 | 26.322 | 1.00 | 35.09 |
| 2986 | CA | GLN | B | 494 | 31.779 | 78.879 | 26.330 | 1.00 | 38.19 |
| 2987 | C | GLN | B | 494 | 32.032 | 80.091 | 25.449 | 1.00 | 36.48 |
| 2988 | O | GLN | B | 494 | 31.360 | 80.270 | 24.432 | 1.00 | 35.75 |
| 2989 | CB | GLN | B | 494 | 32.581 | 77.696 | 25.775 | 1.00 | 43.09 |
| 2990 | CG | GLN | B | 494 | 32.502 | 76.433 | 26.610 | 1.00 | 58.35 |
| 2991 | CD | GLN | B | 494 | 32.929 | 76.666 | 28.042 | 1.00 | 65.11 |
| 2992 | OE1 | GLN | B | 494 | 34.003 | 77.209 | 28.303 | 1.00 | 71.67 |
| 2993 | NE2 | GLN | B | 494 | 32.088 | 76.255 | 28.982 | 1.00 | 71.87 |
| 2994 | N | PRO | B | 495 | 33.002 | 80.939 | 25.828 | 1.00 | 36.89 |
| 2995 | CA | PRO | B | 495 | 33.322 | 82.130 | 25.037 | 1.00 | 38.37 |
| 2996 | C | PRO | B | 495 | 33.609 | 81.737 | 23.587 | 1.00 | 40.80 |
| 2997 | O | PRO | B | 495 | 34.314 | 80.763 | 23.327 | 1.00 | 40.76 |
| 2998 | CB | PRO | B | 495 | 34.557 | 82.680 | 25.729 | 1.00 | 37.83 |
| 2999 | CG | PRO | B | 495 | 34.315 | 82.300 | 27.174 | 1.00 | 40.41 |
| 3000 | CD | PRO | B | 495 | 33.809 | 80.884 | 27.061 | 1.00 | 35.81 |
| 3001 | N | ARG | B | 496 | 33.040 | 82.480 | 22.650 | 1.00 | 42.05 |
| 3002 | CA | ARG | B | 496 | 33.255 | 82.208 | 21.240 | 1.00 | 47.69 |
| 3003 | C | ARG | B | 496 | 33.772 | 83.454 | 20.542 | 1.00 | 51.66 |
| 3004 | O | ARG | B | 496 | 33.283 | 84.558 | 20.784 | 1.00 | 51.95 |
| 3005 | CB | ARG | B | 496 | 31.954 | 81.751 | 20.584 | 1.00 | 45.81 |
| 3006 | CG | ARG | B | 496 | 31.599 | 80.317 | 20.892 | 1.00 | 51.53 |
| 3007 | CD | ARG | B | 496 | 30.217 | 79.947 | 20.391 | 1.00 | 53.72 |
| 3008 | NE | ARG | B | 496 | 30.175 | 78.554 | 19.963 | 1.00 | 57.50 |
| 3009 | CZ | ARG | B | 496 | 30.608 | 78.122 | 18.783 | 1.00 | 58.59 |
| 3010 | NH1 | ARG | B | 496 | 31.111 | 78.974 | 17.901 | 1.00 | 57.22 |
| 3011 | NH2 | ARG | B | 496 | 30.542 | 76.832 | 18.488 | 1.00 | 65.31 |
| 3012 | N | LYS | B | 497 | 34.773 | 83.281 | 19.687 | 1.00 | 56.98 |
| 3013 | CA | LYS | B | 497 | 35.335 | 84.408 | 18.953 | 1.00 | 61.91 |
| 3014 | C | LYS | B | 497 | 34.301 | 84.867 | 17.955 | 1.00 | 63.97 |
| 3015 | O | LYS | B | 497 | 33.300 | 84.195 | 17.729 | 1.00 | 64.61 |
| 3016 | CB | LYS | B | 497 | 36.587 | 84.003 | 18.172 | 1.00 | 66.35 |
| 3017 | CG | LYS | B | 497 | 37.848 | 83.781 | 18.987 | 1.00 | 74.14 |
| 3018 | CD | LYS | B | 497 | 38.993 | 83.359 | 18.066 | 1.00 | 80.69 |
| 3019 | CE | LYS | B | 497 | 40.304 | 83.192 | 18.814 | 1.00 | 84.48 |
| 3020 | NZ | LYS | B | 497 | 41.385 | 82.739 | 17.889 | 1.00 | 88.26 |
| 3021 | N | THR | B | 498 | 34.555 | 86.017 | 17.352 | 1.00 | 67.67 |
| 3022 | CA | THR | B | 498 | 33.669 | 86.572 | 16.342 | 1.00 | 71.26 |
| 3023 | C | THR | B | 498 | 34.550 | 87.284 | 15.340 | 1.00 | 73.04 |
| 3024 | O | THR | B | 498 | 35.743 | 86.990 | 15.236 | 1.00 | 72.83 |
| 3025 | CB | THR | B | 498 | 32.679 | 87.585 | 16.945 | 1.00 | 71.80 |
| 3026 | OG1 | THR | B | 498 | 33.392 | 88.555 | 17.721 | 1.00 | 71.58 |
| 3027 | CG2 | THR | B | 498 | 31.670 | 86.878 | 17.819 | 1.00 | 73.61 |
| 3028 | N | LYS | B | 499 | 33.962 | 88.220 | 14.605 | 1.00 | 75.57 |
| 3029 | CA | LYS | B | 499 | 34.705 | 88.993 | 13.620 | 1.00 | 77.62 |
| 3030 | C | LYS | B | 499 | 35.427 | 90.142 | 14.321 | 1.00 | 77.92 |
| 3031 | O | LYS | B | 499 | 34.791 | 91.096 | 14.768 | 1.00 | 79.19 |
| 3032 | CB | LYS | B | 499 | 33.752 | 89.558 | 12.567 | 1.00 | 80.22 |
| 3033 | CG | LYS | B | 499 | 33.172 | 88.531 | 11.604 | 1.00 | 81.04 |
| 3034 | CD | LYS | B | 499 | 34.203 | 88.093 | 10.571 | 1.00 | 83.26 |
| 3035 | CE | LYS | B | 499 | 33.569 | 87.232 | 9.487 | 1.00 | 84.81 |
| 3036 | NZ | LYS | B | 499 | 34.545 | 86.864 | 8.425 | 1.00 | 86.04 |
| 3037 | N | GLY | B | 500 | 36.748 | 90.034 | 14.433 | 1.00 | 76.91 |
| 3038 | CA | GLY | B | 500 | 37.544 | 91.077 | 15.061 | 1.00 | 75.83 |
| 3039 | C | GLY | B | 500 | 37.141 | 91.560 | 16.445 | 1.00 | 75.14 |
| 3040 | O | GLY | B | 500 | 37.971 | 91.576 | 17.355 | 1.00 | 75.52 |
| 3041 | N | SER | B | 501 | 35.882 | 91.958 | 16.605 | 1.00 | 73.08 |
| 3042 | CA | SER | B | 501 | 35.370 | 92.468 | 17.875 | 1.00 | 71.65 |
| 3043 | C | SER | B | 501 | 36.008 | 91.836 | 19.106 | 1.00 | 68.64 |
| 3044 | O | SER | B | 501 | 36.690 | 92.515 | 19.877 | 1.00 | 71.13 |
| 3045 | CB | SER | B | 501 | 33.854 | 92.279 | 17.953 | 1.00 | 74.43 |
| 3046 | OG | SER | B | 501 | 33.524 | 90.933 | 18.241 | 1.00 | 80.92 |
| 3047 | N | GLY | B | 502 | 35.786 | 90.540 | 19.290 | 1.00 | 63.14 |
| 3048 | CA | GLY | B | 502 | 36.344 | 89.859 | 20.443 | 1.00 | 54.62 |
| 3049 | C | GLY | B | 502 | 35.552 | 88.618 | 20.803 | 1.00 | 48.04 |
| 3050 | O | GLY | B | 502 | 35.469 | 87.683 | 20.006 | 1.00 | 48.08 |
| 3051 | N | PHE | B | 503 | 34.958 | 88.599 | 21.994 | 1.00 | 40.50 |
| 3052 | CA | PHE | B | 503 | 34.189 | 87.427 | 22.408 | 1.00 | 33.81 |
| 3053 | C | PHE | B | 503 | 32.773 | 87.692 | 22.888 | 1.00 | 28.39 |
| 3054 | O | PHE | B | 503 | 32.408 | 88.811 | 23.244 | 1.00 | 26.35 |
| 3055 | CB | PHE | B | 503 | 34.919 | 86.662 | 23.517 | 1.00 | 35.09 |
| 3056 | CG | PHE | B | 503 | 36.263 | 86.148 | 23.119 | 1.00 | 34.16 |
| 3057 | CD1 | PHE | B | 503 | 37.381 | 86.970 | 23.174 | 1.00 | 35.05 |
| 3058 | CD2 | PHE | B | 503 | 36.411 | 84.845 | 22.670 | 1.00 | 35.90 |
| 3059 | CE1 | PHE | B | 503 | 38.633 | 86.501 | 22.783 | 1.00 | 36.00 |
| 3060 | CE2 | PHE | B | 503 | 37.656 | 84.362 | 22.274 | 1.00 | 40.04 |
| 3061 | CZ | PHE | B | 503 | 38.771 | 85.192 | 22.329 | 1.00 | 38.82 |
| 3062 | N | PHE | B | 504 | 31.975 | 86.633 | 22.884 | 1.00 | 26.16 |
| 3063 | CA | PHE | B | 504 | 30.612 | 86.709 | 23.369 | 1.00 | 25.00 |
| 3064 | C | PHE | B | 504 | 30.286 | 85.418 | 24.096 | 1.00 | 26.57 |
| 3065 | O | PHE | B | 504 | 30.892 | 84.379 | 23.833 | 1.00 | 26.73 |
| 3066 | CB | PHE | B | 504 | 29.616 | 86.960 | 22.242 | 1.00 | 22.46 |
| 3067 | CG | PHE | B | 504 | 29.330 | 85.766 | 21.382 | 1.00 | 27.70 |
| 3068 | CD1 | PHE | B | 504 | 28.260 | 84.926 | 21.667 | 1.00 | 28.94 |
| 3069 | CD2 | PHE | B | 504 | 30.086 | 85.517 | 20.243 | 1.00 | 31.34 |
| 3070 | CE1 | PHE | B | 504 | 27.938 | 83.857 | 20.827 | 1.00 | 30.08 |
| 3071 | CE2 | PHE | B | 504 | 29.772 | 84.449 | 19.396 | 1.00 | 32.65 |
| 3072 | CZ | PHE | B | 504 | 28.691 | 83.621 | 19.697 | 1.00 | 32.80 |
| 3073 | N | VAL | B | 505 | 29.330 | 85.509 | 25.015 | 1.00 | 23.68 |
| 3074 | CA | VAL | B | 505 | 28.895 | 84.389 | 25.814 | 1.00 | 24.67 |
| 3075 | C | VAL | B | 505 | 27.385 | 84.559 | 26.028 | 1.00 | 23.86 |
| 3076 | O | VAL | B | 505 | 26.875 | 85.672 | 26.051 | 1.00 | 21.24 |
| 3077 | CB | VAL | B | 505 | 29.674 | 84.392 | 27.161 | 1.00 | 27.95 |
| 3078 | CG1 | VAL | B | 505 | 29.136 | 85.466 | 28.080 | 1.00 | 25.61 |
| 3079 | CG2 | VAL | B | 505 | 29.643 | 83.042 | 27.786 | 1.00 | 33.51 |
| 3080 | N | PHE | B | 506 | 26.664 | 83.454 | 26.141 | 1.00 | 26.20 |
| 3081 | CA | PHE | B | 506 | 25.218 | 83.514 | 26.346 | 1.00 | 26.10 |
| 3082 | C | PHE | B | 506 | 24.855 | 82.754 | 27.621 | 1.00 | 24.83 |
| 3083 | O | PHE | B | 506 | 25.369 | 81.672 | 27.859 | 1.00 | 25.52 |
| 3084 | CB | PHE | B | 506 | 24.496 | 82.875 | 25.161 | 1.00 | 28.18 |
| 3085 | CG | PHE | B | 506 | 23.007 | 82.842 | 25.303 | 1.00 | 35.33 |
| 3086 | CD1 | PHE | B | 506 | 22.245 | 83.980 | 25.080 | 1.00 | 42.56 |
| 3087 | CD2 | PHE | B | 506 | 22.359 | 81.663 | 25.657 | 1.00 | 40.04 |
| 3088 | CE1 | PHE | B | 506 | 20.854 | 83.940 | 25.208 | 1.00 | 44.70 |
| 3089 | CE2 | PHE | B | 506 | 20.977 | 81.613 | 25.789 | 1.00 | 40.54 |
| 3090 | CZ | PHE | B | 506 | 20.223 | 82.749 | 25.564 | 1.00 | 42.12 |
| 3091 | N | SER | B | 507 | 23.961 | 83.310 | 28.427 | 1.00 | 24.69 |
| 3092 | CA | SER | B | 507 | 23.550 | 82.659 | 29.674 | 1.00 | 24.73 |
| 3093 | C | SER | B | 507 | 22.022 | 82.535 | 29.745 | 1.00 | 26.40 |
| 3094 | O | SER | B | 507 | 21.308 | 83.487 | 29.491 | 1.00 | 25.99 |
| 3095 | CB | SER | B | 507 | 24.079 | 83.449 | 30.879 | 1.00 | 22.19 |
| 3096 | OG | SER | B | 507 | 23.653 | 82.860 | 32.091 | 1.00 | 30.30 |
| 3097 | N | ARG | B | 508 | 21.538 | 81.345 | 30.091 | 1.00 | 26.98 |
| 3098 | CA | ARG | B | 508 | 20.104 | 81.055 | 30.187 | 1.00 | 25.95 |
| 3099 | C | ARG | B | 508 | 19.689 | 80.749 | 31.637 | 1.00 | 24.97 |
| 3100 | O | ARG | B | 508 | 20.350 | 79.979 | 32.315 | 1.00 | 24.35 |
| 3101 | CB | ARG | B | 508 | 19.799 | 79.845 | 29.310 | 1.00 | 21.97 |
| 3102 | CG | ARG | B | 508 | 18.365 | 79.351 | 29.344 | 1.00 | 25.32 |
| 3103 | CD | ARG | B | 508 | 18.228 | 78.126 | 28.444 | 1.00 | 25.67 |
| 3104 | NE | ARG | B | 508 | 16.842 | 77.702 | 28.312 | 1.00 | 36.00 |
| 3105 | CZ | ARG | B | 508 | 16.243 | 76.843 | 29.128 | 1.00 | 36.06 |
| 3106 | NH1 | ARG | B | 508 | 16.918 | 76.307 | 30.137 | 1.00 | 34.19 |
| 3107 | NH2 | ARG | B | 508 | 14.967 | 76.538 | 28.943 | 1.00 | 34.70 |
| 3108 | N | LEU | B | 509 | 18.583 | 81.331 | 32.099 | 1.00 | 26.44 |
| 3109 | CA | LEU | B | 509 | 18.121 | 81.108 | 33.479 | 1.00 | 28.45 |
| 3110 | C | LEU | B | 509 | 16.627 | 80.806 | 33.560 | 1.00 | 28.87 |
| 3111 | O | LEU | B | 509 | 15.803 | 81.707 | 33.401 | 1.00 | 30.99 |
| 3112 | CB | LEU | B | 509 | 18.423 | 82.341 | 34.342 | 1.00 | 27.28 |
| 3113 | CG | LEU | B | 509 | 17.867 | 82.330 | 35.779 | 1.00 | 31.90 |
| 3114 | CD1 | LEU | B | 509 | 18.615 | 81.316 | 36.647 | 1.00 | 29.48 |
| 3115 | CD2 | LEU | B | 509 | 18.001 | 83.714 | 36.368 | 1.00 | 28.89 |
| 3116 | N | GLU | B | 510 | 16.277 | 79.546 | 33.812 | 1.00 | 31.92 |
| 3117 | CA | GLU | B | 510 | 14.872 | 79.152 | 33.911 | 1.00 | 34.67 |
| 3118 | C | GLU | B | 510 | 14.310 | 79.595 | 35.255 | 1.00 | 36.35 |
| 3119 | O | GLU | B | 510 | 14.948 | 79.401 | 36.288 | 1.00 | 34.66 |
| 3120 | CB | GLU | B | 510 | 14.725 | 77.630 | 33.757 | 1.00 | 41.07 |
| 3121 | CG | GLU | B | 510 | 15.217 | 77.090 | 32.404 | 1.00 | 54.35 |
| 3122 | CD | GLU | B | 510 | 14.925 | 75.603 | 32.189 | 1.00 | 58.86 |
| 3123 | OE1 | GLU | B | 510 | 15.236 | 74.789 | 33.087 | 1.00 | 61.22 |
| 3124 | OE2 | GLU | B | 510 | 14.393 | 75.251 | 31.111 | 1.00 | 61.34 |
| 3125 | N | VAL | B | 511 | 13.121 | 80.194 | 35.248 | 1.00 | 36.50 |
| 3126 | CA | VAL | B | 511 | 12.531 | 80.665 | 36.494 | 1.00 | 38.05 |
| 3127 | C | VAL | B | 511 | 11.084 | 80.230 | 36.692 | 1.00 | 42.41 |
| 3128 | O | VAL | B | 511 | 10.395 | 79.856 | 35.732 | 1.00 | 41.92 |
| 3129 | CB | VAL | B | 511 | 12.588 | 82.197 | 36.592 | 1.00 | 31.84 |
| 3130 | CG1 | VAL | B | 511 | 14.015 | 82.673 | 36.431 | 1.00 | 32.42 |
| 3131 | CG2 | VAL | B | 511 | 11.687 | 82.813 | 35.547 | 1.00 | 26.87 |
| 3132 | N | THR | B | 512 | 10.633 | 80.290 | 37.946 | 1.00 | 43.58 |
| 3133 | CA | THR | B | 512 | 9.269 | 79.903 | 38.292 | 1.00 | 48.37 |
| 3134 | C | THR | B | 512 | 8.294 | 80.998 | 37.899 | 1.00 | 50.53 |
| 3135 | O | THR | B | 512 | 8.706 | 82.113 | 37.584 | 1.00 | 51.77 |
| 3136 | CB | THR | B | 512 | 9.125 | 79.644 | 39.797 | 1.00 | 48.57 |
| 3137 | OG1 | THR | B | 512 | 9.460 | 80.834 | 40.519 | 1.00 | 46.96 |
| 3138 | CG2 | THR | B | 512 | 10.046 | 78.515 | 40.234 | 1.00 | 45.82 |
| 3139 | N | ARG | B | 513 | 7.002 | 80.678 | 37.912 | 1.00 | 53.29 |
| 3140 | CA | ARG | B | 513 | 5.971 | 81.648 | 37.556 | 1.00 | 55.54 |
| 3141 | C | ARG | B | 513 | 6.043 | 82.819 | 38.523 | 1.00 | 55.38 |
| 3142 | O | ARG | B | 513 | 5.859 | 83.977 | 38.138 | 1.00 | 55.22 |
| 3143 | CB | ARG | B | 513 | 4.582 | 81.006 | 37.636 | 1.00 | 59.23 |
| 3144 | CG | ARG | B | 513 | 3.563 | 81.601 | 36.672 | 1.00 | 65.98 |
| 3145 | CD | ARG | B | 513 | 3.326 | 83.081 | 36.916 | 1.00 | 74.12 |
| 3146 | NE | ARG | B | 513 | 2.773 | 83.742 | 35.735 | 1.00 | 80.97 |
| 3147 | CZ | ARG | B | 513 | 2.403 | 85.019 | 35.687 | 1.00 | 83.64 |
| 3148 | NH1 | ARG | B | 513 | 2.516 | 85.791 | 36.760 | 1.00 | 86.02 |
| 3149 | NH2 | ARG | B | 513 | 1.927 | 85.528 | 34.557 | 1.00 | 85.44 |
| 3150 | N | ALA | B | 514 | 6.315 | 82.501 | 39.783 | 1.00 | 55.03 |
| 3151 | CA | ALA | B | 514 | 6.414 | 83.504 | 40.832 | 1.00 | 55.46 |
| 3152 | C | ALA | B | 514 | 7.496 | 84.539 | 40.529 | 1.00 | 56.10 |
| 3153 | O | ALA | B | 514 | 7.210 | 85.734 | 40.426 | 1.00 | 56.61 |
| 3154 | CB | ALA | B | 514 | 6.692 | 82.825 | 42.173 | 1.00 | 54.05 |
| 3155 | N | GLU | B | 515 | 8.741 | 84.091 | 40.388 | 1.00 | 55.46 |
| 3156 | CA | GLU | B | 515 | 9.819 | 85.025 | 40.105 | 1.00 | 55.34 |
| 3157 | C | GLU | B | 515 | 9.649 | 85.651 | 38.729 | 1.00 | 54.83 |
| 3158 | O | GLU | B | 515 | 10.190 | 86.723 | 38.456 | 1.00 | 52.33 |
| 3159 | CB | GLU | B | 515 | 11.187 | 84.335 | 40.232 | 1.00 | 57.76 |
| 3160 | CG | GLU | B | 515 | 11.221 | 82.884 | 39.788 | 1.00 | 62.58 |
| 3161 | CD | GLU | B | 515 | 12.518 | 82.184 | 40.163 | 1.00 | 63.27 |
| 3162 | OE1 | GLU | B | 515 | 13.035 | 82.432 | 41.271 | 1.00 | 64.89 |
| 3163 | OE2 | GLU | B | 515 | 13.014 | 81.372 | 39.359 | 1.00 | 66.59 |
| 3164 | N | THR | B | 516 | 8.884 | 84.992 | 37.866 | 1.00 | 55.79 |
| 3165 | CA | THR | B | 516 | 8.644 | 85.528 | 36.534 | 1.00 | 57.81 |
| 3166 | C | THR | B | 516 | 7.778 | 86.782 | 36.636 | 1.00 | 58.51 |
| 3167 | O | THR | B | 516 | 7.778 | 87.612 | 35.726 | 1.00 | 57.71 |
| 3168 | CB | THR | B | 516 | 7.951 | 84.499 | 35.634 | 1.00 | 61.48 |
| 3169 | CG | THR | B | 516 | 8.056 | 84.862 | 34.187 | 1.00 | 66.54 |
| 3170 | CD1 | THR | B | 516 | 9.172 | 84.777 | 33.403 | 1.00 | 67.26 |
| 3171 | CD2 | THR | B | 516 | 7.039 | 85.460 | 33.375 | 1.00 | 69.56 |
| 3172 | NE1 | THR | B | 516 | 8.915 | 85.291 | 32.154 | 1.00 | 71.76 |
| 3173 | CE2 | THR | B | 516 | 7.613 | 85.718 | 32.110 | 1.00 | 70.88 |
| 3174 | CE3 | THR | B | 516 | 5.700 | 85.807 | 33.593 | 1.00 | 70.94 |
| 3175 | CZ2 | THR | B | 516 | 6.894 | 86.308 | 31.066 | 1.00 | 73.50 |
| 3176 | CZ3 | THR | B | 516 | 4.982 | 86.396 | 32.552 | 1.00 | 74.37 |
| 3177 | CH2 | THR | B | 516 | 5.584 | 86.639 | 31.304 | 1.00 | 74.71 |
| 3178 | N | GLU | B | 517 | 7.047 | 86.918 | 37.744 | 1.00 | 59.66 |
| 3179 | CA | GLU | B | 517 | 6.191 | 88.085 | 37.972 | 1.00 | 61.72 |
| 3180 | C | GLU | B | 517 | 7.035 | 89.299 | 38.354 | 1.00 | 59.87 |
| 3181 | O | GLU | B | 517 | 6.606 | 90.441 | 38.196 | 1.00 | 58.60 |
| 3182 | CB | GLU | B | 517 | 5.182 | 87.818 | 39.097 | 1.00 | 68.82 |
| 3183 | CG | GLU | B | 517 | 4.284 | 86.612 | 38.879 | 1.00 | 80.60 |
| 3184 | CD | GLU | B | 517 | 3.242 | 86.452 | 39.976 | 1.00 | 87.42 |
| 3185 | OE1 | GLU | B | 517 | 3.369 | 87.128 | 41.021 | 1.00 | 94.80 |
| 3186 | OE2 | GLU | B | 517 | 2.301 | 85.645 | 39.800 | 1.00 | 95.35 |
| 3187 | N | GLU | B | 518 | 8.234 | 89.045 | 38.867 | 1.00 | 57.92 |
| 3188 | CA | GLU | B | 518 | 9.132 | 90.120 | 39.264 | 1.00 | 55.74 |
| 3189 | C | GLU | B | 518 | 10.416 | 90.057 | 38.436 | 1.00 | 54.32 |
| 3190 | O | GLU | B | 518 | 11.498 | 90.393 | 38.924 | 1.00 | 52.85 |
| 3191 | CB | GLU | B | 518 | 9.454 | 90.000 | 40.745 | 1.00 | 54.10 |
| 3192 | N | LYS | B | 519 | 10.287 | 89.646 | 37.176 | 1.00 | 52.69 |
| 3193 | CA | LYS | B | 519 | 11.448 | 89.518 | 36.309 | 1.00 | 52.35 |
| 3194 | C | LYS | B | 519 | 12.206 | 90.819 | 36.159 | 1.00 | 51.69 |
| 3195 | O | LYS | B | 519 | 13.397 | 90.822 | 35.854 | 1.00 | 49.61 |
| 3196 | CB | LYS | B | 519 | 11.039 | 88.983 | 34.937 | 1.00 | 54.31 |
| 3197 | CG | LYS | B | 519 | 10.205 | 89.912 | 34.088 | 1.00 | 55.80 |
| 3198 | CD | LYS | B | 519 | 9.723 | 89.162 | 32.859 | 1.00 | 60.32 |
| 3199 | CE | LYS | B | 519 | 8.794 | 90.002 | 32.011 | 1.00 | 64.05 |
| 3200 | NZ | LYS | B | 519 | 8.223 | 89.191 | 30.898 | 1.00 | 70.92 |
| 3201 | N | ASP | B | 520 | 11.511 | 91.924 | 36.388 | 1.00 | 52.66 |
| 3202 | CA | ASP | B | 520 | 12.105 | 93.251 | 36.288 | 1.00 | 54.22 |
| 3203 | C | ASP | B | 520 | 13.209 | 93.396 | 37.336 | 1.00 | 53.07 |
| 3204 | O | ASP | B | 520 | 14.075 | 94.264 | 37.229 | 1.00 | 52.75 |
| 3205 | CE | ASP | B | 520 | 11.032 | 94.311 | 36.537 | 1.00 | 61.14 |
| 3206 | CG | ASP | B | 520 | 9.620 | 93.779 | 36.308 | 1.00 | 71.19 |
| 3207 | OD1 | ASP | B | 520 | 9.300 | 93.407 | 35.155 | 1.00 | 76.53 |
| 3208 | OD2 | ASP | B | 520 | 8.834 | 93.729 | 37.283 | 1.00 | 72.86 |
| 3209 | N | GLU | B | 521 | 13.177 | 92.533 | 38.343 | 1.00 | 51.01 |
| 3210 | CA | GLU | B | 521 | 14.157 | 92.582 | 39.416 | 1.00 | 50.49 |
| 3211 | C | GLU | B | 521 | 15.394 | 91.714 | 39.201 | 1.00 | 47.73 |
| 3212 | O | GLU | B | 521 | 16.372 | 91.838 | 39.935 | 1.00 | 48.58 |
| 3213 | CB | GLU | B | 521 | 13.493 | 92.200 | 40.738 | 1.00 | 53.54 |
| 3214 | CG | GLU | B | 521 | 12.427 | 93.177 | 41.199 | 1.00 | 60.11 |
| 3215 | CD | GLU | B | 521 | 11.705 | 92.699 | 42.442 | 1.00 | 65.41 |
| 3216 | OE1 | GLU | B | 521 | 12.361 | 92.497 | 43.486 | 1.00 | 67.98 |
| 3217 | OE2 | GLU | B | 521 | 10.471 | 92.521 | 42.374 | 1.00 | 73.58 |
| 3218 | N | PHE | B | 522 | 15.359 | 90.828 | 38.213 | 1.00 | 42.66 |
| 3219 | CA | PHE | B | 522 | 16.519 | 89.985 | 37.963 | 1.00 | 38.90 |
| 3220 | C | PHE | B | 522 | 17.583 | 90.753 | 37.208 | 1.00 | 35.93 |
| 3221 | O | PHE | B | 522 | 17.276 | 91.579 | 36.345 | 1.00 | 38.85 |
| 3222 | CB | PHE | B | 522 | 16.122 | 88.716 | 37.214 | 1.00 | 36.56 |
| 3223 | CG | PHE | B | 522 | 15.419 | 87.710 | 38.081 | 1.00 | 39.04 |
| 3224 | CD1 | PHE | B | 522 | 14.106 | 87.927 | 38.495 | 1.00 | 42.28 |
| 3225 | CD2 | PHE | B | 522 | 16.087 | 86.574 | 38.539 | 1.00 | 36.78 |
| 3226 | CE1 | PHE | B | 522 | 13.468 | 87.029 | 39.362 | 1.00 | 44.30 |
| 3227 | CE2 | PHE | B | 522 | 15.461 | 85.673 | 39.405 | 1.00 | 40.92 |
| 3228 | CZ | PHE | B | 522 | 14.146 | 85.901 | 39.817 | 1.00 | 39.51 |
| 3229 | N | ILE | B | 523 | 18.837 | 90.492 | 37.555 | 1.00 | 31.27 |
| 3230 | CA | ILE | B | 523 | 19.959 | 91.173 | 36.944 | 1.00 | 29.65 |
| 3231 | C | ILE | B | 523 | 21.049 | 90.211 | 36.483 | 1.00 | 29.13 |
| 3232 | O | ILE | B | 523 | 21.360 | 89.236 | 37.164 | 1.00 | 27.36 |
| 3233 | CB | ILE | B | 523 | 20.567 | 92.180 | 37.937 | 1.00 | 34.21 |
| 3234 | CG1 | ILE | B | 523 | 19.528 | 93.251 | 38.282 | 1.00 | 37.38 |
| 3235 | CG2 | ILE | B | 523 | 21.827 | 92.796 | 37.364 | 1.00 | 34.94 |
| 3236 | CD1 | ILE | B | 523 | 19.981 | 94.227 | 39.354 | 1.00 | 38.56 |
| 3237 | N | CYS | B | 524 | 21.610 | 90.498 | 35.311 | 1.00 | 26.34 |
| 3238 | CA | CYS | B | 524 | 22.686 | 89.699 | 34.739 | 1.00 | 27.10 |
| 3239 | C | CYS | B | 524 | 23.946 | 90.500 | 34.962 | 1.00 | 26.67 |
| 3240 | O | CYS | B | 524 | 24.010 | 91.655 | 34.569 | 1.00 | 27.58 |
| 3241 | CB | CYS | B | 524 | 22.473 | 89.497 | 33.226 | 1.00 | 24.27 |
| 3242 | SG | CYS | B | 524 | 23.823 | 88.626 | 32.363 | 1.00 | 37.60 |
| 3243 | N | ARG | B | 525 | 24.951 | 89.886 | 35.572 | 1.00 | 26.37 |
| 3244 | CA | ARG | B | 525 | 26.194 | 90.576 | 35.856 | 1.00 | 26.35 |
| 3245 | C | ARG | B | 525 | 27.419 | 89.887 | 35.304 | 1.00 | 26.72 |
| 3246 | O | ARG | B | 525 | 27.561 | 88.671 | 35.400 | 1.00 | 29.04 |
| 3247 | CB | ARG | B | 525 | 26.376 | 90.748 | 37.361 | 1.00 | 29.81 |
| 3248 | CG | ARG | B | 525 | 27.713 | 91.393 | 37.728 | 1.00 | 35.40 |
| 3249 | CD | ARG | B | 525 | 27.652 | 92.058 | 39.099 | 1.00 | 46.51 |
| 3250 | NE | ARG | B | 525 | 27.796 | 91.113 | 40.193 | 1.00 | 50.77 |
| 3251 | CZ | ARG | B | 525 | 27.461 | 91.379 | 41.451 | 1.00 | 55.09 |
| 3252 | NH1 | ARG | B | 525 | 26.951 | 92.565 | 41.763 | 1.00 | 54.58 |
| 3253 | NH2 | ARG | B | 525 | 27.650 | 90.464 | 42.397 | 1.00 | 56.77 |
| 3254 | N | ALA | B | 526 | 28.311 | 90.680 | 34.731 | 1.00 | 25.38 |
| 3255 | CA | ALA | B | 526 | 29.528 | 90.142 | 34.180 | 1.00 | 25.38 |
| 3256 | C | ALA | B | 526 | 30.721 | 90.728 | 34.925 | 1.00 | 25.99 |
| 3257 | O | ALA | B | 526 | 30.751 | 91.933 | 35.210 | 1.00 | 25.50 |
| 3258 | CB | ALA | B | 526 | 29.629 | 90.484 | 32.696 | 1.00 | 25.98 |
| 3259 | N | VAL | B | 527 | 31.684 | 89.872 | 35.259 | 1.00 | 23.84 |
| 3260 | CA | VAL | B | 527 | 32.895 | 90.333 | 35.902 | 1.00 | 25.15 |
| 3261 | C | VAL | B | 527 | 33.972 | 90.078 | 34.880 | 1.00 | 26.30 |
| 3262 | O | VAL | B | 527 | 34.119 | 88.958 | 34.394 | 1.00 | 26.20 |
| 3263 | CB | VAL | B | 527 | 33.235 | 89.557 | 37.177 | 1.00 | 24.43 |
| 3264 | CG1 | VAL | B | 527 | 34.588 | 89.990 | 37.661 | 1.00 | 26.95 |
| 3265 | CG2 | VAL | B | 527 | 32.191 | 89.819 | 38.251 | 1.00 | 28.13 |
| 3266 | N | HIS | B | 528 | 34.738 | 91.115 | 34.569 | 1.00 | 26.24 |
| 3267 | CA | HIS | B | 528 | 35.772 | 91.018 | 33.564 | 1.00 | 29.14 |
| 3268 | C | HIS | B | 528 | 36.822 | 92.102 | 33.805 | 1.00 | 31.86 |
| 3269 | O | HIS | B | 528 | 36.509 | 93.216 | 34.239 | 1.00 | 31.05 |
| 3270 | CB | HIS | B | 528 | 35.121 | 91.172 | 32.179 | 1.00 | 29.03 |
| 3271 | CG | HIS | B | 528 | 36.077 | 91.082 | 31.035 | 1.00 | 29.34 |
| 3272 | ND1 | HIS | B | 528 | 36.934 | 92.109 | 30.696 | 1.00 | 33.33 |
| 3273 | CD2 | HIS | B | 528 | 36.317 | 90.090 | 30.144 | 1.00 | 28.93 |
| 3274 | CE1 | HIS | B | 528 | 37.655 | 91.755 | 29.651 | 1.00 | 26.07 |
| 3275 | NE2 | HIS | B | 528 | 37.302 | 90.530 | 29.295 | 1.00 | 32.19 |
| 3276 | N | GLU | B | 529 | 38.064 | 91.757 | 33.501 | 1.00 | 34.08 |
| 3277 | CA | GLU | B | 529 | 39.207 | 92.636 | 33.676 | 1.00 | 39.76 |
| 3278 | C | GLU | B | 529 | 39.110 | 94.037 | 33.066 | 1.00 | 41.50 |
| 3279 | O | GLU | B | 529 | 39.410 | 95.020 | 33.740 | 1.00 | 41.48 |
| 3280 | CB | GLU | B | 529 | 40.462 | 91.959 | 33.122 | 1.00 | 43.03 |
| 3281 | CG | GLU | B | 529 | 41.665 | 92.898 | 33.086 | 1.00 | 56.19 |
| 3282 | CD | GLU | B | 529 | 42.827 | 92.361 | 32.274 | 1.00 | 62.66 |
| 3283 | OE1 | GLU | B | 529 | 43.812 | 93.111 | 32.093 | 1.00 | 63.52 |
| 3284 | OE2 | GLU | B | 529 | 42.758 | 91.198 | 31.819 | 1.00 | 65.43 |
| 3285 | N | ALA | B | 530 | 38.703 | 94.121 | 31.800 | 1.00 | 42.51 |
| 3286 | CA | ALA | B | 530 | 38.622 | 95.399 | 31.094 | 1.00 | 46.03 |
| 3287 | C | ALA | B | 530 | 37.495 | 96.331 | 31.504 | 1.00 | 49.29 |
| 3288 | O | ALA | B | 530 | 37.447 | 97.476 | 31.056 | 1.00 | 50.87 |
| 3289 | CB | ALA | B | 530 | 38.557 | 95.157 | 29.587 | 1.00 | 39.71 |
| 3290 | N | ALA | B | 531 | 36.595 | 95.849 | 32.352 | 1.00 | 52.89 |
| 3291 | CA | ALA | B | 531 | 35.467 | 96.651 | 32.798 | 1.00 | 56.14 |
| 3292 | C | ALA | B | 531 | 35.801 | 97.570 | 33.976 | 1.00 | 59.66 |
| 3293 | O | ALA | B | 531 | 35.357 | 97.342 | 35.096 | 1.00 | 61.00 |
| 3294 | CB | ALA | B | 531 | 34.305 | 95.736 | 33.162 | 1.00 | 52.83 |
| 3295 | N | SER | B | 532 | 36.588 | 98.608 | 33.730 | 1.00 | 63.07 |
| 3296 | CA | SER | B | 532 | 36.927 | 99.544 | 34.795 | 1.00 | 66.03 |
| 3297 | C | SER | B | 532 | 35.822 | 100.594 | 34.850 | 1.00 | 66.08 |
| 3298 | O | SER | B | 532 | 35.317 | 101.031 | 33.814 | 1.00 | 65.35 |
| 3299 | CB | SER | B | 532 | 38.270 | 100.216 | 34.507 | 1.00 | 68.98 |
| 3300 | OG | SER | B | 532 | 38.184 | 101.074 | 33.384 | 1.00 | 74.64 |
| 3301 | N | PRO | B | 533 | 35.427 | 101.019 | 36.057 | 1.00 | 66.51 |
| 3302 | CA | PRO | B | 533 | 35.887 | 100.669 | 37.404 | 1.00 | 65.99 |
| 3303 | C | PRO | B | 533 | 35.374 | 99.349 | 37.983 | 1.00 | 63.48 |
| 3304 | O | PRO | B | 533 | 34.327 | 98.840 | 37.582 | 1.00 | 64.33 |
| 3305 | CB | PRO | B | 533 | 35.397 | 101.843 | 38.227 | 1.00 | 69.06 |
| 3306 | CG | PRO | B | 533 | 34.044 | 102.061 | 37.629 | 1.00 | 71.08 |
| 3307 | CD | PRO | B | 533 | 34.358 | 102.030 | 36.137 | 1.00 | 70.23 |
| 3308 | N | SER | B | 534 | 36.134 | 98.817 | 38.935 | 1.00 | 59.21 |
| 3309 | CA | SER | B | 534 | 35.796 | 97.591 | 39.648 | 1.00 | 55.01 |
| 3310 | C | SER | B | 534 | 35.452 | 96.326 | 38.852 | 1.00 | 50.27 |
| 3311 | O | SER | B | 534 | 34.725 | 95.470 | 39.347 | 1.00 | 48.58 |
| 3312 | CB | SER | B | 534 | 34.666 | 97.894 | 40.625 | 1.00 | 58.20 |
| 3313 | OG | SER | B | 534 | 33.587 | 98.526 | 39.962 | 1.00 | 63.36 |
| 3314 | N | GLN | B | 535 | 35.984 | 96.213 | 37.639 | 1.00 | 44.89 |
| 3315 | CA | GLN | B | 535 | 35.771 | 95.057 | 36.770 | 1.00 | 42.88 |
| 3316 | C | GLN | B | 535 | 34.395 | 94.406 | 36.823 | 1.00 | 41.55 |
| 3317 | O | GLN | B | 535 | 34.279 | 93.181 | 36.806 | 1.00 | 40.80 |
| 3318 | CB | GLN | B | 535 | 36.831 | 93.967 | 37.020 | 1.00 | 37.08 |
| 3319 | CG | GLN | B | 535 | 37.869 | 94.298 | 38.044 | 1.00 | 47.40 |
| 3320 | CD | GLN | B | 535 | 38.684 | 95.499 | 37.669 | 1.00 | 44.36 |
| 3321 | OE1 | GLN | B | 535 | 38.862 | 96.407 | 38.475 | 1.00 | 47.59 |
| 3322 | NE2 | GLN | B | 535 | 39.185 | 95.521 | 36.436 | 1.00 | 54.70 |
| 3323 | N | THR | B | 536 | 33.348 | 95.213 | 36.874 | 1.00 | 39.97 |
| 3324 | CA | THR | B | 536 | 32.012 | 94.650 | 36.893 | 1.00 | 40.62 |
| 3325 | C | THR | B | 536 | 31.025 | 95.487 | 36.077 | 1.00 | 37.57 |
| 3326 | O | THR | B | 536 | 31.042 | 96.707 | 36.125 | 1.00 | 40.85 |
| 3327 | CB | THR | B | 536 | 31.508 | 94.463 | 38.353 | 1.00 | 42.38 |
| 3328 | OG1 | THR | B | 536 | 30.112 | 94.142 | 38.337 | 1.00 | 44.59 |
| 3329 | CG2 | THR | B | 536 | 31.730 | 95.719 | 39.168 | 1.00 | 49.65 |
| 3330 | N | VAL | B | 537 | 30.190 | 94.812 | 35.299 | 1.00 | 35.29 |
| 3331 | CA | VAL | B | 537 | 29.182 | 95.453 | 34.468 | 1.00 | 31.25 |
| 3332 | C | VAL | B | 537 | 27.922 | 94.604 | 34.563 | 1.00 | 29.01 |
| 3333 | O | VAL | B | 537 | 27.985 | 93.382 | 34.401 | 1.00 | 30.11 |
| 3334 | CB | VAL | B | 537 | 29.604 | 95.494 | 32.983 | 1.00 | 34.80 |
| 3335 | CG1 | VAL | B | 537 | 28.516 | 96.148 | 32.161 | 1.00 | 35.52 |
| 3336 | CG2 | VAL | B | 537 | 30.888 | 96.259 | 32.822 | 1.00 | 37.19 |
| 3337 | N | GLN | B | 538 | 26.780 | 95.235 | 34.808 | 1.00 | 26.73 |
| 3338 | CA | GLN | B | 538 | 25.533 | 94.493 | 34.933 | 1.00 | 26.74 |
| 3339 | C | GLN | B | 538 | 24.329 | 95.224 | 34.368 | 1.00 | 27.53 |
| 3340 | O | GLN | B | 538 | 24.351 | 96.431 | 34.198 | 1.00 | 29.89 |
| 3341 | CB | GLN | B | 538 | 25.265 | 94.176 | 36.400 | 1.00 | 28.48 |
| 3342 | CG | GLN | B | 538 | 24.976 | 95.392 | 37.266 | 1.00 | 25.88 |
| 3343 | CD | GLN | B | 538 | 24.747 | 95.005 | 38.722 | 1.00 | 28.51 |
| 3344 | OE1 | GLN | B | 538 | 25.498 | 94.210 | 39.290 | 1.00 | 31.66 |
| 3345 | NE2 | GLN | B | 538 | 23.715 | 95.565 | 39.328 | 1.00 | 27.39 |
| 3346 | N | ARG | B | 539 | 23.266 | 94.487 | 34.087 | 1.00 | 28.05 |
| 3347 | CA | ARG | B | 539 | 22.070 | 95.107 | 33.565 | 1.00 | 30.72 |
| 3348 | C | ARG | B | 539 | 20.858 | 94.312 | 33.986 | 1.00 | 31.33 |
| 3349 | O | ARG | B | 539 | 20.897 | 93.083 | 34.023 | 1.00 | 32.42 |
| 3350 | CB | ARG | B | 539 | 22.137 | 95.180 | 32.039 | 1.00 | 36.30 |
| 3351 | CG | ARG | B | 539 | 21.124 | 96.112 | 31.414 | 1.00 | 45.60 |
| 3352 | CD | ARG | B | 539 | 21.771 | 96.859 | 30.270 | 1.00 | 57.97 |
| 3353 | NE | ARG | B | 539 | 23.009 | 97.482 | 30.729 | 1.00 | 69.46 |
| 3354 | CZ | ARG | B | 539 | 23.855 | 98.147 | 29.950 | 1.00 | 78.18 |
| 3355 | NH1 | ARG | B | 539 | 23.604 | 98.284 | 28.653 | 1.00 | 83.01 |
| 3356 | NH2 | ARG | B | 539 | 24.959 | 98.673 | 30.470 | 1.00 | 81.89 |
| 3357 | N | ALA | B | 540 | 19.782 | 95.022 | 34.301 | 1.00 | 31.92 |
| 3358 | CA | ALA | B | 540 | 18.546 | 94.393 | 34.717 | 1.00 | 34.50 |
| 3359 | C | ALA | B | 540 | 17.826 | 93.909 | 33.466 | 1.00 | 36.68 |
| 3360 | O | ALA | B | 540 | 18.068 | 94.410 | 32.361 | 1.00 | 34.62 |
| 3361 | CB | ALA | B | 540 | 17.693 | 95.391 | 35.479 | 1.00 | 33.91 |
| 3362 | N | VAL | B | 541 | 16.974 | 92.906 | 33.640 | 1.00 | 39.73 |
| 3363 | CA | VAL | B | 541 | 16.214 | 92.346 | 32.528 | 1.00 | 44.47 |
| 3364 | C | VAL | B | 541 | 15.351 | 93.436 | 31.882 | 1.00 | 46.91 |
| 3365 | O | VAL | B | 541 | 14.476 | 94.005 | 32.527 | 1.00 | 44.53 |
| 3366 | CB | VAL | B | 541 | 15.305 | 91.185 | 33.010 | 1.00 | 42.49 |
| 3367 | CG1 | VAL | B | 541 | 14.242 | 90.885 | 31.970 | 1.00 | 47.96 |
| 3368 | CG2 | VAL | B | 541 | 16.140 | 89.945 | 33.269 | 1.00 | 42.43 |
| 3369 | N | SER | B | 542 | 15.603 | 93.731 | 30.611 | 1.00 | 52.25 |
| 3370 | CA | SER | B | 542 | 14.829 | 94.760 | 29.926 | 1.00 | 60.05 |
| 3371 | C | SER | B | 542 | 13.426 | 94.226 | 29.679 | 1.00 | 64.25 |
| 3372 | O | SER | B | 542 | 13.247 | 93.050 | 29.357 | 1.00 | 65.22 |
| 3373 | CB | SER | B | 542 | 15.490 | 95.157 | 28.600 | 1.00 | 60.43 |
| 3374 | OG | SER | B | 542 | 15.524 | 94.070 | 27.696 | 1.00 | 65.30 |
| 3375 | N | VAL | B | 543 | 12.432 | 95.090 | 29.844 | 1.00 | 68.19 |
| 3376 | CA | VAL | B | 543 | 11.043 | 94.694 | 29.656 | 1.00 | 72.45 |
| 3377 | C | VAL | B | 543 | 10.277 | 95.772 | 28.898 | 1.00 | 74.32 |
| 3378 | O | VAL | B | 543 | 9.621 | 96.630 | 29.497 | 1.00 | 75.99 |
| 3379 | CB | VAL | B | 543 | 10.353 | 94.452 | 31.016 | 1.00 | 74.41 |
| 3380 | CG1 | VAL | B | 543 | 8.895 | 94.070 | 30.801 | 1.00 | 76.37 |
| 3381 | CG2 | VAL | B | 543 | 11.086 | 93.367 | 31.782 | 1.00 | 75.78 |
| 3382 | O | HOH | B | 1 | 28.242 | 81.091 | 34.909 | 1.00 | 54.75 |
| 3383 | O | HOH | B | 2 | 20.156 | 98.063 | 34.554 | 1.00 | 37.27 |
| 3384 | O | HOH | B | 3 | 9.164 | 79.181 | 26.395 | 1.00 | 46.90 |
| 3385 | O | HOH | B | 4 | 40.362 | 78.148 | 37.537 | 1.00 | 44.93 |
| 3386 | O | HOH | B | 5 | 26.513 | 97.015 | 29.107 | 1.00 | 46.77 |
| 3387 | O | HON | B | 6 | 24.671 | 83.376 | 40.011 | 1.00 | 40.32 |
| 3388 | O | HOH | B | 7 | 50.797 | 88.328 | 45.010 | 1.00 | 50.21 |
| 3389 | O | HOH | B | 8 | 30.596 | 75.260 | 16.415 | 1.00 | 61.75 |
| 3390 | O | HOH | B | 9 | 39.623 | 81.063 | 40.390 | 1.00 | 39.17 |
| 3391 | O | HOH | B | 10 | 10.819 | 88.759 | 44.203 | 1.00 | 64.36 |
| 3392 | O | HOH | B | 11 | 43.896 | 82.650 | 46.479 | 1.00 | 45.74 |
| 3393 | O | HOH | B | 12 | 40.405 | 77.131 | 40.006 | 1.00 | 39.34 |
| 3394 | O | HOH | B | 13 | 28.630 | 51.829 | 41.383 | 1.00 | 56.15 |
| 3395 | O | HOH | B | 14 | 18.197 | 77.472 | 34.045 | 1.00 | 46.48 |
| 3396 | O | HOH | B | 15 | 29.246 | 81.729 | 23.290 | 1.00 | 36.55 |
| 3397 | O | HOH | B | 16 | 27.005 | 76.944 | 32.905 | 1.00 | 48.95 |
| 3398 | O | HOH | B | 17 | 46.365 | 79.161 | 30.402 | 1.00 | 50.90 |
| 3399 | O | HOH | B | 18 | 29.027 | 56.778 | 36.404 | 1.00 | 58.83 |
| 3400 | O | HOH | B | 19 | 32.617 | 59.584 | 31.846 | 1.00 | 61.24 |
| 3401 | O | HOH | B | 20 | 46.447 | 87.589 | 46.492 | 1.00 | 58.37 |
| 3402 | O | HOH | B | 21 | 37.888 | 95.014 | 40.873 | 1.00 | 57.48 |
| 3403 | O | HOH | B | 22 | 55.424 | 66.383 | 26.960 | 1.00 | 60.62 |
| 3404 | O | HOH | B | 23 | 49.953 | 85.610 | 35.164 | 1.00 | 49.14 |
| 3405 | O | HOH | B | 24 | 0.662 | 87.508 | 40.326 | 1.00 | 63.67 |
| 3406 | O | HOH | B | 25 | 19.011 | 94.187 | 29.591 | 1.00 | 40.50 |
| 3407 | O | HOH | B | 26 | 43.242 | 49.660 | 46.237 | 1.00 | 58.12 |
| 3408 | O | HOH | B | 27 | 40.629 | 78.744 | 31.911 | 1.00 | 52.34 |
| 3409 | O | HOH | B | 28 | 40.803 | 72.686 | 34.647 | 1.00 | 52.41 |
| 3410 | O | HOH | B | 29 | 1.434 | 81.662 | 24.774 | 1.00 | 63.47 |
| 3411 | O | HOH | B | 30 | 29.558 | 77.458 | 32.128 | 1.00 | 56.91 |
| 3412 | O | HOH | B | 31 | 40.590 | 70.716 | 43.907 | 1.00 | 49.06 |
| 3413 | O | HOH | B | 32 | 23.070 | 95.434 | 42.368 | 1.00 | 59.31 |
| 3414 | O | HOH | B | 33 | 58.388 | 76.819 | 41.695 | 1.00 | 51.40 |
| 3415 | O | HOH | B | 34 | 50.954 | 79.227 | 44.916 | 1.00 | 49.71 |
| 3416 | O | HOH | B | 35 | 34.068 | 54.729 | 35.446 | 1.00 | 64.98 |
| 3417 | O | HOH | B | 36 | 29.928 | 90.630 | 44.684 | 1.00 | 55.97 |
| 3418 | O | HOH | B | 37 | 29.718 | 98.417 | 28.749 | 1.00 | 56.10 |
| 3419 | O | HOH | B | 38 | 34.139 | 96.711 | 23.956 | 1.00 | 48.69 |
| 3420 | O | HOH | B | 39 | 38.006 | 89.010 | 17.278 | 1.00 | 58.57 |
| 3421 | O | HOH | B | 40 | 52.915 | 67.142 | 28.195 | 1.00 | 54.81 |
| 3422 | O | HOH | B | 41 | 41.555 | 51.194 | 38.581 | 1.00 | 65.02 |
| 3423 | O | HOH | B | 42 | 52.498 | 94.849 | 41.604 | 1.00 | 60.95 |
| 3424 | O | HOH | B | 43 | 23.412 | 53.599 | 37.661 | 1.00 | 59.29 |
| 3425 | O | HOH | B | 44 | 28.241 | 88.615 | 44.746 | 1.00 | 64.70 |
| 3426 | O | HOH | B | 45 | 54.871 | 85.305 | 45.726 | 1.00 | 57.91 |
| 3427 | O | HOH | B | 46 | 9.818 | 81.648 | 43.286 | 1.00 | 54.20 |
| 3428 | O | HOH | B | 47 | 26.359 | 91.470 | 45.088 | 1.00 | 59.40 |
| 3429 | O | HOH | B | 48 | 28.072 | 63.227 | 38.869 | 1.00 | 61.07 |
| 3430 | O | HOH | B | 49 | 45.277 | 55.789 | 32.757 | 1.00 | 62.77 |
| 3431 | O | HOH | B | 50 | 44.758 | 73.013 | 29.503 | 1.00 | 54.90 |
| 3432 | O | HOH | B | 51 | 38.266 | 78.900 | 44.091 | 1.00 | 59.03 |
| 3433 | O | HOH | B | 52 | 8.212 | 78.463 | 34.558 | 1.00 | 57.55 |
| 3434 | O | HOH | B | 53 | 1.381 | 85.874 | 28.805 | 1.00 | 55.02 |
| 3435 | O | HOH | B | 54 | 26.806 | 78.029 | 35.293 | 1.00 | 62.62 |
| 3436 | O | HOH | B | 55 | 12.447 | 95.162 | 25.458 | 1.00 | 64.18 |
| 3437 | O | HOH | B | 56 | 3.811 | 85.411 | 25.179 | 1.00 | 57.80 |
| 3438 | O | HOH | B | 57 | 29.871 | 60.364 | 33.052 | 1.00 | 68.28 |
| 3439 | O | HOH | B | 58 | 19.470 | 76.874 | 31.345 | 1.00 | 42.77 |
| 3440 | O | HOH | B | 59 | 43.684 | 89.839 | 24.236 | 1.00 | 49.12 |
| 3441 | O | HOH | B | 60 | 8.499 | 86.927 | 28.688 | 1.00 | 60.00 |
| 3442 | O | HOH | B | 61 | 42.759 | 54.014 | 38.123 | 1.00 | 66.82 |
| 3443 | O | HOH | B | 62 | 57.282 | 92.935 | 37.270 | 1.00 | 61.58 |
| 3444 | O | HOH | B | 63 | 35.759 | 94.606 | 20.953 | 1.00 | 54.56 |
| 3445 | O | HOH | B | 64 | 55.504 | 71.596 | 38.010 | 1.00 | 52.20 |
| 3446 | O | HOH | B | 65 | 51.524 | 82.672 | 43.463 | 1.00 | 63.55 |
| 3447 | O | HOH | B | 66 | 25.162 | 92.476 | 24.637 | 1.00 | 49.41 |
| 3448 | O | HOH | B | 67 | 20.866 | 91.104 | 50.882 | 1.00 | 59.74 |
| 3449 | O | HOH | B | 68 | 21.567 | 72.397 | 38.063 | 1.00 | 61.59 |
| 3450 | O | HOH | B | 69 | 34.917 | 80.449 | 18.554 | 1.00 | 58.32 |
| 3451 | O | HOH | B | 70 | 58.059 | 62.072 | 40.018 | 1.00 | 67.46 |
| 3452 | O | HOH | B | 71 | 46.678 | 76.624 | 50.973 | 1.00 | 56.29 |
| 3453 | O | HOH | B | 72 | 47.970 | 66.515 | 43.658 | 1.00 | 57.89 |
| 3454 | O | HOH | B | 73 | 58.976 | 70.892 | 35.943 | 1.00 | 61.84 |
| 3455 | O | HOH | B | 74 | 37.925 | 102.300 | 36.267 | 1.00 | 70.53 |
| 3456 | O | HOH | B | 75 | 35.095 | 82.009 | 30.623 | 1.00 | 53.22 |
| 3457 | O | HOH | B | 76 | 48.950 | 89.607 | 45.995 | 1.00 | 60.73 |
| 3458 | O | HOH | B | 77 | 51.049 | 54.014 | 41.456 | 1.00 | 64.35 |
| 3459 | O | HOH | B | 78 | 9.614 | 90.974 | 28.382 | 1.00 | 59.27 |
| 3460 | O | HOH | B | 79 | 30.892 | 81.866 | 34.924 | 1.00 | 53.70 |
| 3461 | O | HOH | B | 80 | 8.623 | 88.500 | 43.305 | 1.00 | 60.32 |
| 3462 | O | HOH | B | 81 | 37.845 | 96.067 | 20.364 | 1.00 | 56.40 |
| 3463 | O | HOH | B | 82 | 38.754 | 86.712 | 18.621 | 1.00 | 62.11 |
| 3464 | O | HOH | B | 83 | 33.854 | 61.761 | 30.984 | 1.00 | 57.66 |
| 3465 | O | HOH | B | 84 | 31.092 | 97.083 | 24.789 | 1.00 | 61.70 |
| 3466 | O | HOH | B | 85 | 55.604 | 63.730 | 25.209 | 1.00 | 61.38 |
| 3467 | O | HOH | B | 86 | 10.681 | 91.038 | 44.382 | 1.00 | 67.86 |
| 3468 | O | HOH | B | 87 | 8.631 | 76.809 | 27.264 | 1.00 | 61.99 |
| 3469 | O | HOH | B | 88 | 27.366 | 82.163 | 39.549 | 1.00 | 54.45 |
| 3470 | O | HOH | B | 89 | 25.286 | 75.986 | 42.777 | 1.00 | 57.86 |
| 3471 | O | HOH | B | 90 | 37.707 | 77.054 | 41.662 | 1.00 | 58.43 |
| 3472 | O | HOH | B | 91 | 32.692 | 58.560 | 34.055 | 1.00 | 67.78 |
| 3473 | O | HOH | B | 92 | 53.944 | 63.193 | 28.977 | 1.00 | 65.22 |
| 3474 | O | HOH | B | 93 | 39.865 | 81.851 | 49.172 | 1.00 | 60.31 |
| 3475 | O | HOH | B | 94 | 31.308 | 61.582 | 30.906 | 1.00 | 61.54 |
| 3476 | O | HOH | B | 95 | 59.514 | 93.999 | 36.788 | 1.00 | 67.64 |
| 3477 | O | HOH | B | 96 | 39.447 | 99.425 | 29.592 | 1.00 | 56.59 |
| 3478 | O | HOH | B | 97 | 58.449 | 74.096 | 43.103 | 1.00 | 64.62 |
| 3479 | O | HOH | B | 98 | 29.674 | 63.288 | 40.778 | 1.00 | 66.03 |
| 3480 | O | HOH | B | 99 | 48.458 | 74.578 | 28.945 | 1.00 | 62.65 |
| 3481 | O | HOH | B | 100 | 1.217 | 87.605 | 43.348 | 1.00 | 63.95 |
| 3482 | O | HOH | B | 101 | 18.899 | 94.884 | 51.591 | 1.00 | 63.86 |
| 3483 | O | HOH | B | 102 | 32.969 | 77.837 | 30.721 | 1.00 | 62.58 |
| 3484 | O | HOH | B | 103 | 0.563 | 90.219 | 39.830 | 1.00 | 58.40 |
| 3485 | O | HOH | B | 104 | 26.801 | 57.949 | 31.582 | 1.00 | 61.34 |
| 3486 | O | HOH | B | 105 | 11.041 | 70.673 | 37.151 | 1.00 | 65.87 |
| 3487 | O | HOH | B | 106 | 22.839 | 96.513 | 26.321 | 1.00 | 61.27 |
| 3488 | O | HOH | B | 107 | 51.734 | 82.918 | 46.361 | 1.00 | 67.17 |
| 3489 | O | HOH | B | 108 | 47.037 | 68.251 | 48.363 | 1.00 | 54.90 |
| 3490 | O | HOH | B | 109 | 7.726 | 80.618 | 44.317 | 1.00 | 66.23 |
| 3491 | O | HOH | B | 110 | 32.588 | 73.719 | 18.127 | 1.00 | 59.96 |
| 3492 | O | HOH | B | 111 | 44.611 | 57.647 | 45.310 | 1.00 | 64.92 |
| 3493 | O | HOH | B | 112 | 32.288 | 63.471 | 36.999 | 1.00 | 65.68 |
| 3494 | O | HOH | B | 113 | 47.059 | 68.725 | 45.458 | 1.00 | 61.47 |
| 3495 | O | HOH | B | 114 | 52.648 | 70.274 | 44.235 | 1.00 | 60.20 |
| 3496 | O | HOH | B | 115 | 28.226 | 99.464 | 26.888 | 1.00 | 59.29 |
| 3497 | O | HOH | B | 116 | 10.671 | 79.859 | 28.221 | 1.00 | 53.05 |
| 3498 | O | HOH | B | 117 | 47.224 | 51.238 | 37.567 | 1.00 | 69.15 |
| 3499 | O | HOH | B | 118 | 2.077 | 82.274 | 29.937 | 1.00 | 62.28 |
| 3500 | O | HOH | B | 119 | 21.625 | 99.622 | 31.504 | 1.00 | 59.61 |
| 3501 | O | HOH | B | 120 | 51.285 | 72.528 | 46.379 | 1.00 | 61.94 |
| 3502 | O | HOH | B | 121 | 60.228 | 68.403 | 35.257 | 1.00 | 64.45 |
| 3503 | O | HOH | B | 122 | 40.261 | 93.766 | 43.138 | 1.00 | 63.01 |
| 3504 | O | HOH | B | 123 | 25.735 | 89.468 | 46.487 | 1.00 | 59.29 |
| 3505 | O | HOH | B | 124 | 31.385 | 92.796 | 46.304 | 1.00 | 61.69 |
| 3506 | O | HOH | B | 125 | 33.401 | 99.434 | 35.006 | 1.00 | 69.10 |
| 3507 | O | HOH | B | 126 | 53.684 | 58.928 | 43.989 | 1.00 | 61.79 |
| 3508 | O | HOH | B | 127 | 48.916 | 83.660 | 48.343 | 1.00 | 63.74 |
| 3509 | O | HOH | B | 128 | 32.974 | 85.797 | 6.578 | 1.00 | 67.04 |
| 3510 | O | HOH | B | 129 | 2.491 | 77.598 | 35.303 | 1.00 | 62.20 |
| 3511 | O | HOH | B | 130 | 39.071 | 89.336 | 19.546 | 1.00 | 66.48 |
| 3512 | O | HOH | B | 131 | 33.906 | 66.916 | 41.602 | 1.00 | 70.79 |
| 3513 | O | HOH | B | 132 | 7.009 | 74.725 | 27.690 | 1.00 | 72.41 |
| 3514 | O | HOH | B | 133 | 41.213 | 85.156 | 25.009 | 1.00 | 62.09 |
| 3515 | O | HOH | B | 134 | 53.407 | 88.641 | 45.536 | 1.00 | 61.32 |
| 3516 | O | HOH | B | 135 | 61.105 | 61.893 | 39.131 | 1.00 | 68.49 |
| 3517 | O | HOH | B | 136 | 54.402 | 78.303 | 50.147 | 1.00 | 69.04 |
| 3518 | O | HOH | B | 137 | 58.078 | 60.598 | 38.009 | 1.00 | 63.49 |
| 3519 | O | HOH | B | 138 | 23.599 | 84.930 | 47.731 | 1.00 | 53.37 |
| 3520 | O | HOH | B | 139 | 5.249 | 86.853 | 27.014 | 1.00 | 59.07 |
| 3521 | O | HOH | B | 140 | 58.796 | 62.599 | 31.318 | 1.00 | 65.35 |
| 3522 | O | HOH | B | 141 | 50.703 | 49.006 | 36.896 | 1.00 | 67.74 |
| 3523 | O | HOH | B | 142 | 11.022 | 88.620 | 46.640 | 1.00 | 67.40 |
| 3524 | O | HOH | B | 143 | 37.589 | 71.107 | 41.445 | 1.00 | 64.43 |
| 3525 | O | HOH | B | 144 | 17.187 | 98.039 | 48.494 | 1.00 | 67.27 |
| 3526 | O | HOH | B | 145 | 30.164 | 50.230 | 40.408 | 1.00 | 70.07 |

As used herein, an atomic coordinate, also referred to herein as a structure coordinate or coordinate, is a mathematical coordinate derived from mathematical equations related to the patterns obtained on diffraction of X-rays by the atoms of a protein or complex crystal. The diffraction data are typically used to calculate an electron density map, which is used to establish the positions of the individual atoms within the unit cell of the crystal. A model that substantially represents the atomic coordinates specified in Table 1, Table 2 or Table 3 includes not only models that literally represent the coordinates but also models representing a coordinate transformation of such atomic coordinates, for example, by changing the spatial orientation of the coordinates.

The present invention also includes a 3-D model that is a modification of a 3-D model that substantially represents the atomic coordinates specified in Table 1, Table 2 or Table 3. As used herein, a modification, also referred to herein as a model modification, is a model that represents an antibody Fc region that binds to a Fc receptor protein. A model modification includes, but is not limited to: a refinement of the model that substantially represents the atomic coordinates specified in Table 1, Table 2 or Table 3; a model representing any FcR-binding fragment of an antibody having the atomic coordinates specified in Table 1, Table 2 or Table 3; a model based on other Fc-Cε3/Cε4 crystals, such as a model based on a crystal disclosed in the Examples; a model produced using homology modeling techniques to, for example, incorporate all or any part of the amino acid sequence of another Fc region into a 3-D model substantially representing the atomic coordinates specified in Table 1, Table 2 or Table 3 or incorporate all or any part of the amino acid sequence of a Fc-Cε3/Cε4 into a 3-D model of another antibody; and a modification representing a Fc region that has an altered function, which preferably can be used to design a mutein with an improved function compared to an unmodified protein. As used herein, the term unmodified protein refers to a protein that has not been intentionally subjected to either random or site-directed (i.e., targeted) mutagenesis. While not being bound by theory, it is believed that the flexibility of the Cε3 and Cε4 chains of the Fc region of IgE which allows the formation of open (receptor-bound) and closed conformations, can also lead to other dynamic conformations, all of which are included in the present invention. Such flexibility is also a target for identification of development of compounds to inhibit binding of IgE to its receptor. In one embodiment, the distance between two Cε3 domains of a Fc region of the present invention ranges from about 10 angstroms to about 25 angstroms. In another embodiment, the distance between two Cε3 domains of a Fc region of the present invention ranges from about 20 angstroms to about 40 angstroms, with a range of from about 20 to about 30 angstroms being preferred.

A model of the present invention can be represented in a variety of forms including, but not limited to, listing the coordinates of all atoms comprising the model, providing a physical 3-D model, imaging the model on a computer screen, providing a picture of said model, and deriving a set of coordinates based of a picture of the model, for example by extracting coordinates from a picture or placing a similar immunoglobulin domain into the 3-D model of a human Fc-Cε3/Cε4₂₂₂ protein having SEQ ID NO:2 and deriving a model of the similar domain. Physical 3-D models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Evans and Sutherland, Salt Lake City, Utah, Biosym Technologies, San Diego, Calif., Tripos, Inc., and Molecular Simulations Inc. The phrase “providing a picture of the model” refers to the ability to generate a “hard copy” of the model. Hard copies include both motion and still pictures. Computer screen images and pictures of the model can be visualized in a number of formats including, but not limited to, electron density maps, ribbon diagrams, space-filling representations, a carbon traces, topology diagrams, lists of interatomic vectors, phi/psi/chi angle representations of the coordinates, and contact maps, examples of some of which are in the Figs. Representations of the model can include the entire model or portions thereof. A model can also be represented in a database.

A model of the present invention also defines the space surrounding that model. Such a space can be represented as a mold, or alpha-space, that can be used to predict the shape of a compound that inhibits the binding of a FcR and antibody.

In one embodiment, a model of the present invention identifies the solvent accessibility of amino acid residues of the corresponding proteins in the complex. The solvent accessibilities of the amino acids in PhFc-Cε3/Cε4₁₋₂₂₂ are indicated in Table 4.

TABLE 4
IgE-Fc Residue Exposure
Surface plot for:
structure file = 7_more_easy.mtf
coordinate set = 7_more_easy.pdb

| | | | average accessible area | | |
|---|---|---|---|---|---|
| segid | resid | resname | residue | mainchain | sidechain |
| A | 336 | VAL | 11.0355 | 10.7157 | 11.4619 |
| A | 337 | SER | 6.6644 | 0.9102 | 18.1728 |
| A | 338 | ALA | 1.5503 | 1.9379 | 0.0000 |
| A | 339 | TYR | 8.1765 | 1.1049 | 11.7123 |
| A | 340 | LEU | 3.6795 | 7.1662 | 0.1928 |
| A | 341 | SER | 10.1371 | 1.1968 | 28.0177 |
| A | 342 | ARG | 6.2194 | 3.9259 | 7.5300 |
| A | 343 | PRO | 1.0168 | 1.4058 | 0.4982 |
| A | 344 | SER | 7.7961 | 2.0652 | 19.2580 |
| A | 345 | PRO | 1.3218 | 0.0070 | 3.0748 |
| A | 346 | PHE | 4.9773 | 0.0000 | 7.8215 |
| A | 347 | ASP | 3.9717 | 0.0000 | 7.9435 |
| A | 348 | LEU | 2.0606 | 0.7880 | 3.3332 |
| A | 349 | PHE | 4.1860 | 4.1923 | 4.1824 |
| A | 350 | ILE | 8.0920 | 2.3463 | 13.8377 |
| A | 351 | ARG | 12.2182 | 6.3068 | 15.5962 |
| A | 352 | LYS | 15.4391 | 4.6705 | 24.0540 |
| A | 353 | SER | 8.3827 | 1.7463 | 21.6553 |
| A | 354 | PRO | 0.3971 | 0.3072 | 0.5168 |
| A | 355 | THR | 5.7285 | 1.2045 | 11.7605 |
| A | 356 | ILE | 0.1440 | 0.2879 | 0.0000 |
| A | 357 | THR | 5.3215 | 0.3410 | 11.9621 |
| A | 358 | CYS | 0.0000 | 0.0000 | 0.0000 |
| A | 359 | LEU | 3.5251 | 0.0000 | 7.0503 |
| A | 360 | VAL | 0.5274 | 0.0632 | 1.1465 |
| A | 361 | VAL | 3.5236 | 0.1093 | 8.0760 |
| A | 362 | ASP | 1.8760 | 0.2987 | 3.4534 |
| A | 363 | ALA | 20.1207 | 10.3102 | 59.3628 |
| A | 364 | ALA | 15.4855 | 4.7737 | 58.3324 |
| A | 365 | PRO | 8.8456 | 4.2563 | 14.9647 |
| A | 366 | ALA | 19.4281 | 10.6196 | 54.6617 |
| A | 367 | LYS | 14.1954 | 9.3656 | 18.0592 |
| A | 368 | GLY | 19.0191 | 19.0191 | 0.0000 |
| A | 369 | ALA | 13.5479 | 3.8042 | 52.5228 |
| A | 370 | VAL | 1.9022 | 0.2211 | 4.1436 |
| A | 371 | ASN | 9.8425 | 0.1360 | 19.5491 |
| A | 372 | LEU | 3.3729 | 6.1984 | 0.5473 |
| A | 373 | THR | 11.0885 | 1.1749 | 24.3065 |
| A | 374 | TRP | 1.6039 | 3.8874 | 0.6904 |
| A | 375 | SER | 8.4426 | 0.6753 | 23.9772 |
| A | 376 | ARG | 3.0964 | 1.9387 | 3.7580 |
| A | 377 | ALA | 15.7221 | 11.6308 | 32.0872 |
| A | 378 | SER | 11.3325 | 9.7475 | 14.5025 |
| A | 379 | GLY | 16.4108 | 16.4108 | 0.0000 |
| A | 380 | LYS | 13.6283 | 1.9326 | 22.9849 |
| A | 381 | PRO | 15.4265 | 3.6481 | 31.1310 |
| A | 382 | VAL | 7.3416 | 6.0250 | 9.0971 |
| A | 383 | ASN | 11.3782 | 3.3857 | 19.3706 |
| A | 384 | HIS | 17.2932 | 3.2244 | 26.6723 |
| A | 385 | SER | 9.0295 | 7.7970 | 11.4946 |
| A | 386 | THR | 10.4147 | 0.8671 | 23.1449 |
| A | 387 | ARG | 8.9842 | 6.7673 | 10.2510 |
| A | 388 | LYS | 13.1006 | 1.8914 | 22.0680 |
| A | 389 | GLU | 12.0438 | 8.6316 | 14.7736 |
| A | 390 | ALA | 12.8310 | 2.6317 | 53.6284 |
| A | 391 | ALA | 26.6304 | 21.5969 | 46.7642 |
| A | 397 | LEU | 7.6007 | 6.7288 | 8.4727 |
| A | 398 | THR | 5.6519 | 0.0606 | 13.1069 |
| A | 399 | VAL | 0.3919 | 0.0000 | 0.9144 |
| A | 400 | THR | 3.3881 | 0.0000 | 7.9056 |
| A | 401 | SER | 0.6660 | 0.0000 | 1.9979 |
| A | 402 | THR | 3.4801 | 0.2334 | 7.8090 |
| A | 403 | LEU | 0.1292 | 0.0003 | 0.2581 |
| A | 404 | PRO | 8.0051 | 0.8129 | 17.5947 |
| A | 405 | VAL | 1.7566 | 3.0741 | 0.0000 |
| A | 406 | GLY | 7.5226 | 7.5226 | 0.0000 |
| A | 407 | THR | 7.4670 | 1.2753 | 15.7226 |
| A | 408 | ALA | 11.6207 | 1.1130 | 53.6516 |
| A | 409 | ASP | 7.3957 | 0.7299 | 14.0615 |
| A | 410 | TRP | 0.6616 | 0.0000 | 0.9263 |
| A | 411 | ILE | 12.9785 | 6.2633 | 19.6936 |
| A | 412 | GLU | 16.8133 | 9.6352 | 22.5559 |
| A | 413 | GLY | 9.4666 | 9.4666 | 0.0000 |
| A | 414 | GLU | 2.5510 | 2.5156 | 2.5794 |
| A | 415 | THR | 5.9752 | 0.0898 | 13.8224 |
| A | 416 | TYR | 0.5074 | 0.0226 | 0.7498 |
| A | 417 | GLN | 7.2197 | 0.0821 | 12.9297 |
| A | 418 | CYS | 0.0003 | 0.0000 | 0.0009 |
| A | 419 | ALA | 5.4324 | 0.0002 | 27.1613 |
| A | 420 | VAL | 0.0672 | 0.1174 | 0.0002 |
| A | 421 | THR | 9.2655 | 0.3477 | 21.1559 |
| A | 422 | ALA | 3.5044 | 2.8442 | 6.1452 |
| A | 423 | PRO | 8.5866 | 10.2103 | 6.4216 |
| A | 424 | ALA | 8.3566 | 3.5672 | 27.5142 |
| A | 425 | LEU | 11.7212 | 8.7651 | 14.6773 |
| A | 426 | PRO | 17.7261 | 10.5785 | 27.2562 |
| A | 427 | ARG | 17.5755 | 2.8707 | 25.9782 |
| A | 428 | ALA | 9.8149 | 5.3119 | 27.8269 |
| A | 429 | LEU | 6.4318 | 0.7950 | 12.0687 |
| A | 430 | MET | 15.6753 | 5.2815 | 26.0691 |
| A | 431 | ARG | 8.2369 | 1.4606 | 12.1090 |
| A | 432 | SER | 12.1223 | 6.7546 | 22.8576 |
| A | 433 | THR | 1.7085 | 1.7868 | 1.6042 |
| A | 434 | THR | 7.5244 | 1.8978 | 15.0265 |
| A | 435 | ALA | 7.7753 | 3.7138 | 24.0213 |
| A | 436 | THR | 8.6934 | 2.5690 | 16.8594 |
| A | 437 | SER | 15.8041 | 3.9341 | 39.5441 |
| A | 438 | GLY | 10.8151 | 10.8151 | 0.0000 |
| A | 439 | PRO | 13.4383 | 3.2705 | 26.9955 |
| A | 440 | ARG | 8.7641 | 7.5143 | 9.4782 |
| A | 441 | ALA | 6.7461 | 1.8371 | 26.3821 |
| A | 442 | ALA | 10.4272 | 3.2972 | 38.9468 |
| A | 443 | PRO | 1.3762 | 2.4083 | 0.0000 |
| A | 444 | ALA | 9.1593 | 2.0797 | 37.4777 |
| A | 445 | VAL | 0.9848 | 1.7231 | 0.0005 |
| A | 446 | TYR | 3.1461 | 0.0000 | 4.7192 |
| A | 447 | ALA | 0.0289 | 0.0362 | 0.0000 |
| A | 448 | PHE | 0.6153 | 0.0000 | 0.9669 |
| A | 449 | ALA | 3.8692 | 3.2275 | 6.4360 |
| A | 450 | THR | 1.9237 | 0.4303 | 3.9148 |
| A | 451 | PRO | 12.1453 | 6.5643 | 19.5866 |
| A | 452 | GLU | 7.9403 | 6.9431 | 8.7381 |
| A | 453 | ALA | 30.2649 | 20.0784 | 71.0111 |
| A | 459 | LYS | 14.7292 | 11.5275 | 17.2907 |
| A | 460 | ARG | 5.9981 | 0.0765 | 9.3819 |
| A | 461 | THR | 0.1536 | 0.0000 | 0.3584 |
| A | 462 | LEU | 0.0001 | 0.0002 | 0.0000 |
| A | 463 | ALA | 0.0001 | 0.0001 | 0.0000 |
| A | 464 | CYS | 0.0087 | 0.0130 | 0.0000 |
| A | 465 | LEU | 0.0982 | 0.1233 | 0.0732 |
| A | 466 | ILE | 0.0200 | 0.0000 | 0.0401 |
| A | 467 | GLN | 0.8305 | 0.0002 | 1.4947 |
| A | 468 | ASN | 5.9937 | 1.5627 | 10.4246 |
| A | 469 | PHE | 0.0000 | 0.0000 | 0.0000 |
| A | 470 | MET | 7.2629 | 0.0000 | 14.5258 |
| A | 471 | PRO | 1.8851 | 1.4594 | 2.4527 |
| A | 472 | GLU | 7.3364 | 0.4665 | 12.8323 |
| A | 473 | ASP | 5.6153 | 1.4927 | 9.7380 |
| A | 474 | ILE | 3.5567 | 6.3075 | 0.8058 |
| A | 475 | SER | 2.6057 | 1.5404 | 4.7363 |
| A | 476 | VAL | 2.4253 | 3.4886 | 1.0077 |
| A | 477 | GLN | 1.0498 | 0.0000 | 1.8897 |
| A | 478 | TRP | 0.1187 | 0.2438 | 0.0687 |
| A | 479 | LEU | 0.6608 | 0.0000 | 1.3216 |
| A | 480 | HIS | 5.1342 | 0.7731 | 8.0417 |
| A | 481 | ASN | 6.5819 | 0.8724 | 12.2914 |
| A | 482 | GLU | 18.7049 | 10.4001 | 25.3487 |
| A | 483 | VAL | 12.0619 | 0.7436 | 27.1528 |
| A | 484 | GLN | 8.9474 | 5.5610 | 11.6565 |
| A | 485 | LEU | 3.1319 | 1.3283 | 4.9354 |
| A | 486 | PRO | 12.0736 | 3.3535 | 23.7005 |
| A | 487 | ASP | 12.6169 | 1.7276 | 23.5062 |
| A | 488 | ALA | 20.4452 | 11.0832 | 57.8933 |
| A | 489 | ARG | 7.8562 | 4.5508 | 9.7450 |
| A | 490 | HIS | 2.6987 | 4.4236 | 1.5488 |
| A | 491 | SER | 4.4347 | 1.1905 | 10.9232 |
| A | 492 | THR | 5.2960 | 2.8369 | 8.5749 |
| A | 493 | THR | 0.9440 | 0.7955 | 1.1420 |
| A | 494 | GLN | 13.6713 | 0.9109 | 23.8796 |
| A | 495 | PRO | 6.0581 | 4.4972 | 8.1393 |
| A | 496 | ARG | 6.3674 | 0.9523 | 9.4618 |
| A | 497 | LYS | 16.8451 | 3.4562 | 27.5561 |
| A | 498 | THR | 4.5143 | 6.9964 | 1.2047 |
| A | 499 | ALA | 17.0252 | 11.1864 | 40.3805 |
| A | 500 | GLY | 18.8178 | 18.8178 | 0.0000 |
| A | 501 | SER | 5.4365 | 3.4214 | 9.4666 |
| A | 502 | GLY | 2.8894 | 2.8894 | 0.0000 |
| A | 503 | PHE | 3.0581 | 0.0894 | 4.7545 |
| A | 504 | PHE | 0.0656 | 0.0228 | 0.0900 |
| A | 505 | VAL | 0.2424 | 0.0053 | 0.5586 |
| A | 506 | PHE | 0.0016 | 0.0039 | 0.0003 |
| A | 507 | SER | 0.0350 | 0.0179 | 0.0692 |
| A | 508 | ARG | 0.6294 | 0.6714 | 0.6054 |
| A | 509 | LEU | 0.0392 | 0.0185 | 0.0599 |
| A | 510 | GLU | 1.7287 | 1.2093 | 2.1442 |
| A | 511 | VAL | 0.3299 | 0.5773 | 0.0000 |
| A | 512 | THR | 10.6447 | 1.4856 | 22.8569 |
| A | 513 | ARG | 9.5285 | 0.8196 | 14.5051 |
| A | 514 | ALA | 14.8713 | 4.4288 | 56.6415 |
| A | 515 | GLU | 3.0827 | 1.0010 | 4.7481 |
| A | 516 | TRP | 3.3932 | 0.5950 | 4.5125 |
| A | 517 | GLU | 12.8907 | 5.2405 | 19.0109 |
| A | 518 | ALA | 5.1774 | 0.5587 | 23.6525 |
| A | 519 | LYS | 3.0790 | 0.0000 | 5.5422 |
| A | 520 | ASP | 10.4782 | 3.6427 | 17.3137 |
| A | 521 | GLU | 7.1047 | 0.7149 | 12.2165 |
| A | 522 | PHE | 0.0000 | 0.0000 | 0.0001 |
| A | 523 | ILE | 3.5408 | 0.0000 | 7.0817 |
| A | 524 | CYS | 0.0000 | 0.0000 | 0.0000 |
| A | 525 | ARG | 4.6100 | 0.0000 | 7.2442 |
| A | 526 | ALA | 0.0902 | 0.1128 | 0.0000 |
| A | 527 | VAL | 0.0669 | 0.0002 | 0.1558 |
| A | 528 | HIS | 0.1300 | 0.0054 | 0.2131 |
| A | 529 | GLU | 7.9604 | 2.2867 | 12.4993 |
| A | 530 | ALA | 4.2802 | 4.7817 | 2.2743 |
| A | 531 | ALA | 0.7347 | 0.7875 | 0.5235 |
| A | 532 | SER | 15.8770 | 7.2992 | 33.0325 |
| A | 533 | PRO | 12.8146 | 2.0699 | 27.1409 |
| A | 534 | SER | 13.2955 | 4.9891 | 29.9083 |
| A | 535 | GLN | 4.3097 | 0.0000 | 7.7574 |
| A | 536 | THR | 5.4579 | 3.7045 | 7.7958 |
| A | 537 | VAL | 5.1081 | 1.4055 | 10.0450 |
| A | 538 | GLN | 8.2068 | 4.5359 | 11.1435 |
| A | 539 | ARG | 10.8337 | 1.0576 | 16.4201 |
| A | 540 | ALA | 9.1994 | 1.8430 | 38.6251 |
| A | 541 | VAL | 1.0570 | 1.8498 | 0.0000 |
| A | 542 | SER | 8.0549 | 3.4723 | 17.2202 |
| A | 543 | VAL | 18.5723 | 20.6424 | 15.8122 |
| A | 2 | NAG | 16.4353 | 0.0000 | 16.4353 |
| B | 336 | VAL | 10.7710 | 10.1163 | 11.6438 |
| B | 337 | SER | 6.7303 | 0.9166 | 18.3577 |
| B | 338 | ALA | 2.1458 | 2.6822 | 0.0000 |
| B | 339 | TYR | 8.0696 | 1.1676 | 11.5206 |
| B | 340 | LEU | 3.9394 | 7.4662 | 0.4127 |
| B | 341 | SER | 10.1645 | 1.0695 | 28.3545 |
| B | 342 | ARG | 6.0757 | 3.9259 | 7.3041 |
| B | 343 | PRO | 1.0733 | 1.5124 | 0.4878 |
| B | 344 | SER | 7.7643 | 2.0338 | 19.2253 |
| B | 345 | PRO | 1.3185 | 0.0201 | 3.0497 |
| B | 346 | PHE | 4.8258 | 0.0000 | 7.5834 |
| B | 347 | ASP | 3.9197 | 0.0000 | 7.8394 |
| B | 348 | LEU | 1.9999 | 0.7895 | 3.2104 |
| B | 349 | PHE | 4.1817 | 4.2283 | 4.1551 |
| B | 350 | ILE | 7.9930 | 2.5475 | 13.4385 |
| B | 351 | ARG | 12.3730 | 6.6042 | 15.6695 |
| B | 352 | LYS | 15.0045 | 3.9474 | 23.8501 |
| B | 353 | SER | 8.5948 | 1.6503 | 22.4838 |
| B | 354 | PRO | 0.2553 | 0.1754 | 0.3617 |
| B | 355 | THR | 5.9200 | 1.2321 | 12.1705 |
| B | 356 | ILE | 0.1391 | 0.2781 | 0.0000 |
| B | 357 | THR | 5.2617 | 0.3654 | 11.7900 |
| B | 358 | CYS | 0.0000 | 0.0000 | 0.0000 |
| B | 359 | LEU | 3.5340 | 0.0000 | 7.0680 |
| B | 360 | VAL | 0.5390 | 0.0799 | 1.1511 |
| B | 361 | VAL | 3.7224 | 0.1093 | 8.5398 |
| B | 362 | ASP | 1.8094 | 0.3525 | 3.2662 |
| B | 363 | ALA | 20.0439 | 10.4606 | 58.3772 |
| B | 364 | ALA | 15.3707 | 4.9576 | 57.0231 |
| B | 365 | PRO | 9.1152 | 4.3450 | 15.4754 |
| B | 366 | ALA | 18.8285 | 10.0035 | 54.1287 |
| B | 367 | LYS | 14.1725 | 9.5150 | 17.8985 |
| B | 368 | GLY | 19.0630 | 19.0630 | 0.0000 |
| B | 369 | ALA | 13.0115 | 3.7971 | 49.8693 |
| B | 370 | VAL | 1.7523 | 0.1998 | 3.8225 |
| B | 371 | ASN | 10.0509 | 0.2812 | 19.8206 |
| B | 372 | LEU | 3.4036 | 6.2343 | 0.5729 |
| B | 373 | THR | 11.1077 | 0.8564 | 24.7761 |
| B | 374 | TRP | 1.5073 | 3.6124 | 0.6652 |
| B | 375 | SER | 8.3615 | 0.6614 | 23.7617 |
| B | 376 | ARG | 3.0771 | 1.9562 | 3.7176 |
| B | 377 | ALA | 15.1815 | 11.3744 | 30.4099 |
| B | 378 | SER | 11.4636 | 9.9129 | 14.5651 |
| B | 379 | GLY | 16.5804 | 16.5804 | 0.0000 |
| B | 380 | LYS | 13.2366 | 1.9343 | 22.2785 |
| B | 381 | PRO | 15.6715 | 3.5998 | 31.7671 |
| B | 382 | VAL | 7.5283 | 6.2163 | 9.2776 |
| B | 383 | ASN | 11.4020 | 3.4390 | 19.3649 |
| B | 384 | HIS | 17.6888 | 3.0947 | 27.4182 |
| B | 385 | SER | 9.0145 | 7.7907 | 11.4622 |
| B | 386 | THR | 10.6406 | 1.1756 | 23.2606 |
| B | 387 | ARG | 8.8774 | 6.6756 | 10.1356 |
| B | 388 | LYS | 12.6102 | 1.7918 | 21.2649 |
| B | 389 | GLU | 12.4839 | 8.7255 | 15.4906 |
| B | 390 | ALA | 12.8869 | 2.5604 | 54.1927 |
| B | 391 | ALA | 27.0786 | 21.9964 | 47.4075 |
| B | 397 | LEU | 7.6502 | 6.6415 | 8.6588 |
| B | 398 | THR | 5.7838 | 0.0392 | 13.4434 |
| B | 399 | VAL | 0.3391 | 0.0000 | 0.7912 |
| B | 400 | THR | 3.3805 | 0.0000 | 7.8879 |
| B | 401 | SER | 0.7193 | 0.0000 | 2.1579 |
| B | 402 | THR | 3.7007 | 0.2432 | 8.3106 |
| B | 403 | LEU | 0.1106 | 0.0003 | 0.2208 |
| B | 404 | PRO | 8.1540 | 0.8611 | 17.8779 |
| B | 405 | VAL | 1.5566 | 2.7241 | 0.0000 |
| B | 406 | GLY | 7.1791 | 7.1791 | 0.0000 |
| B | 407 | THR | 7.0853 | 0.8445 | 15.4063 |
| B | 408 | ALA | 12.2481 | 1.5500 | 55.0403 |
| B | 409 | ASP | 7.3052 | 0.7230 | 13.8873 |
| B | 410 | TRP | 0.6460 | 0.0006 | 0.9042 |
| B | 411 | ILE | 12.7563 | 5.8221 | 19.6905 |
| B | 412 | GLU | 16.8891 | 9.7584 | 22.5936 |
| B | 413 | GLY | 9.7410 | 9.7410 | 0.0000 |
| B | 414 | GLU | 2.5752 | 2.6119 | 2.5458 |
| B | 415 | THR | 5.4692 | 0.0703 | 12.6676 |
| B | 416 | TYR | 0.4512 | 0.0209 | 0.6663 |
| B | 417 | GLN | 7.0959 | 0.0872 | 12.7029 |
| B | 418 | CYS | 0.0000 | 0.0000 | 0.0000 |
| B | 419 | ALA | 5.4563 | 0.0000 | 27.2815 |
| B | 420 | VAL | 0.0843 | 0.1418 | 0.0075 |
| B | 421 | THR | 9.3197 | 0.3238 | 21.3142 |
| B | 422 | ALA | 3.6164 | 3.0205 | 6.0000 |
| B | 423 | PRO | 8.4785 | 10.2004 | 6.1826 |
| B | 424 | ALA | 8.6226 | 3.5932 | 28.7401 |
| B | 425 | LEU | 11.9869 | 8.8343 | 15.1395 |
| B | 426 | PRO | 17.5822 | 10.4491 | 27.0930 |
| B | 427 | ARG | 17.4020 | 2.6803 | 25.8144 |
| B | 428 | ALA | 9.7570 | 5.0751 | 28.4846 |
| B | 429 | LEU | 6.0642 | 0.7211 | 11.4073 |
| B | 430 | MET | 16.0818 | 5.9456 | 26.2180 |
| B | 431 | ARG | 8.0744 | 1.6123 | 11.7671 |
| B | 432 | SER | 12.2372 | 6.6046 | 23.5024 |
| B | 433 | THR | 1.4684 | 1.6247 | 1.2601 |
| B | 434 | THR | 7.3739 | 1.2286 | 15.5676 |
| B | 435 | ALA | 7.8667 | 3.8619 | 23.8858 |
| B | 436 | THR | 8.4287 | 2.5153 | 16.3133 |
| B | 437 | SER | 15.9443 | 4.0579 | 39.7171 |
| B | 438 | GLY | 10.6293 | 10.6293 | 0.0000 |
| B | 439 | PRO | 13.9484 | 3.3166 | 28.1241 |
| B | 440 | ARG | 8.8462 | 7.2368 | 9.7658 |
| B | 441 | ALA | 6.9507 | 1.8590 | 27.3179 |
| B | 442 | ALA | 10.4125 | 3.4391 | 38.3062 |
| B | 443 | PRO | 1.4008 | 2.4371 | 0.0192 |
| B | 444 | ALA | 9.0537 | 2.0618 | 37.0209 |
| B | 445 | VAL | 1.0436 | 1.8263 | 0.0000 |
| B | 446 | TYR | 3.2280 | 0.0004 | 4.8418 |
| B | 447 | ALA | 0.0363 | 0.0454 | 0.0000 |
| B | 448 | PHE | 0.6260 | 0.0000 | 0.9837 |
| B | 449 | ALA | 3.9414 | 3.2169 | 6.8396 |
| B | 450 | THR | 2.0045 | 0.4174 | 4.1205 |
| B | 451 | PRO | 12.0887 | 6.8483 | 19.0759 |
| B | 452 | GLU | 8.1587 | 7.1306 | 8.9811 |
| B | 453 | ALA | 30.2751 | 20.7889 | 68.2199 |
| B | 459 | LYS | 14.9002 | 12.1326 | 17.1143 |
| B | 460 | ARG | 6.1453 | 0.0029 | 9.6552 |
| B | 461 | THR | 0.1144 | 0.0006 | 0.2661 |
| B | 462 | LEU | 0.0002 | 0.0003 | 0.0000 |
| B | 463 | ALA | 0.0000 | 0.0000 | 0.0000 |
| B | 464 | CYS | 0.0113 | 0.0170 | 0.0000 |
| B | 465 | LEU | 0.0910 | 0.0946 | 0.0874 |
| B | 466 | ILE | 0.0219 | 0.0000 | 0.0437 |
| B | 467 | GLN | 0.8389 | 0.0000 | 1.5100 |
| B | 468 | ASN | 5.9301 | 1.4721 | 10.3881 |
| B | 469 | PHE | 0.0000 | 0.0000 | 0.0000 |
| B | 470 | MET | 7.0813 | 0.0000 | 14.1626 |
| B | 471 | PRO | 1.8331 | 1.3727 | 2.4469 |
| B | 472 | GLU | 7.3943 | 0.5175 | 12.8958 |
| B | 473 | ASP | 5.5075 | 1.5025 | 9.5124 |
| B | 474 | ILE | 3.5810 | 6.3586 | 0.8033 |
| B | 475 | SER | 2.6182 | 1.5408 | 4.7731 |
| B | 476 | VAL | 2.3682 | 3.3339 | 1.0806 |
| B | 477 | GLN | 1.0871 | 0.0000 | 1.9567 |
| B | 478 | TRP | 0.1016 | 0.2324 | 0.0492 |
| B | 479 | LEU | 0.5440 | 0.0000 | 1.0880 |
| B | 480 | HIS | 5.2031 | 0.7382 | 8.1798 |
| B | 481 | ASN | 6.8401 | 0.8806 | 12.7996 |
| B | 482 | GLU | 19.0404 | 11.1613 | 25.3436 |
| B | 483 | VAL | 11.8885 | 0.8953 | 26.5460 |
| B | 484 | GLN | 8.9330 | 5.5624 | 11.6295 |
| B | 485 | LEU | 2.9872 | 1.1924 | 4.7819 |
| B | 486 | PRO | 12.4420 | 3.2393 | 24.7123 |
| B | 487 | ASP | 12.6235 | 1.8226 | 23.4245 |
| B | 488 | ALA | 20.9557 | 11.2245 | 59.8807 |
| B | 489 | ARG | 7.5032 | 4.6052 | 9.1591 |
| B | 490 | HIS | 2.6761 | 4.4041 | 1.5240 |
| B | 491 | SER | 4.3984 | 1.1740 | 10.8470 |
| B | 492 | THR | 5.2933 | 2.7736 | 8.6528 |
| B | 493 | THR | 0.8019 | 0.6773 | 0.9680 |
| B | 494 | GLN | 13.6817 | 0.8084 | 23.9803 |
| B | 495 | PRO | 5.9741 | 4.5062 | 7.9314 |
| B | 496 | ARG | 6.4874 | 0.9751 | 9.6372 |
| B | 497 | LYS | 16.4562 | 3.2776 | 26.9991 |
| B | 498 | THR | 4.6427 | 7.1707 | 1.2720 |
| B | 499 | ALA | 17.5008 | 11.3708 | 42.0207 |
| B | 500 | GLY | 18.7401 | 18.7401 | 0.0000 |
| B | 501 | SER | 5.4215 | 3.4199 | 9.4248 |
| B | 502 | GLY | 2.8691 | 2.8691 | 0.0000 |
| B | 503 | PHE | 2.9965 | 0.0437 | 4.6838 |
| B | 504 | PHE | 0.0651 | 0.0203 | 0.0907 |
| B | 505 | VAL | 0.2042 | 0.0000 | 0.4765 |
| B | 506 | PHE | 0.0052 | 0.0143 | 0.0000 |
| B | 507 | SER | 0.0331 | 0.0000 | 0.0993 |
| B | 508 | ARG | 0.5554 | 0.6385 | 0.5080 |
| B | 509 | LEU | 0.0476 | 0.0286 | 0.0666 |
| B | 510 | GLU | 1.7117 | 1.1583 | 2.1544 |
| B | 511 | VAL | 0.3510 | 0.6142 | 0.0000 |
| B | 512 | THR | 10.6600 | 1.5410 | 22.8185 |
| B | 513 | ARG | 9.4359 | 0.8028 | 14.3691 |
| B | 514 | ALA | 15.2935 | 4.4804 | 58.5460 |
| B | 515 | GLU | 2.9658 | 1.4237 | 4.1995 |
| B | 516 | TRP | 3.3612 | 0.5404 | 4.4896 |
| B | 517 | GLU | 12.5876 | 4.9796 | 18.6740 |
| B | 518 | ALA | 4.6347 | 0.6025 | 20.7633 |
| B | 519 | LYS | 2.7664 | 0.0000 | 4.9795 |
| B | 520 | ASP | 10.6223 | 3.6037 | 17.6410 |
| B | 521 | GLU | 7.3610 | 0.6603 | 12.7216 |
| B | 522 | PHE | 0.0081 | 0.0000 | 0.0128 |
| B | 523 | ILE | 3.5242 | 0.0007 | 7.0477 |
| B | 524 | CYS | 0.0000 | 0.0000 | 0.0000 |
| B | 525 | ARG | 4.7547 | 0.0000 | 7.4716 |
| B | 526 | ALA | 0.0709 | 0.0887 | 0.0000 |
| B | 527 | VAL | 0.0762 | 0.0000 | 0.1777 |
| B | 528 | HIS | 0.1255 | 0.0005 | 0.2088 |
| B | 529 | GLU | 7.9368 | 2.2949 | 12.4503 |
| B | 530 | ALA | 4.2207 | 4.6880 | 2.3517 |
| B | 531 | ALA | 0.7409 | 0.8075 | 0.4746 |
| B | 532 | SER | 16.1085 | 7.5394 | 33.2468 |
| B | 533 | PRO | 13.1853 | 2.0824 | 27.9892 |
| B | 534 | SER | 13.2835 | 4.9738 | 29.9029 |
| B | 535 | GLN | 4.1001 | 0.0000 | 7.3802 |
| B | 536 | THR | 5.5193 | 3.5752 | 8.1115 |
| B | 537 | VAL | 5.1049 | 1.4593 | 9.9656 |
| B | 538 | GLN | 8.2211 | 4.4767 | 11.2166 |
| B | 539 | ARG | 10.8149 | 1.1022 | 16.3650 |
| B | 540 | ALA | 8.9590 | 1.8529 | 37.3835 |
| B | 541 | VAL | 1.0803 | 1.8905 | 0.0000 |
| B | 542 | SER | 8.4553 | 4.0882 | 17.1897 |
| B | 543 | VAL | 18.5355 | 20.8533 | 15.4451 |
| B | 2 | NAG | 16.5934 | 0.0000 | 16.5934 |

In another embodiment, the solvent accessibilities of the amino acids in PhFc-Cε3/Cε4₁₋₂₂₂ are indicated in Table 5.

TABLE 5
IgE-Fc Residue Exposure
>>>> Surface plot for:
>>>> structure file = 1FP5_easy.mtf
>>>> coordinate set = 1FP5_easy.pdb

| | | | total accessible area | | |
|---|---|---|---|---|---|
| segid | resid | resname | residue | mainchain | sidechain |
| A | 336 | VAL | 64.9492 | 37.4745 | 27.4746 |
| A | 337 | SER | 39.1673 | 3.9332 | 35.2340 |
| A | 338 | ALA | 8.7894 | 8.7894 | 0.0000 |
| A | 339 | TYR | 125.2426 | 4.9472 | 120.2955 |
| A | 340 | LEU | 29.8639 | 28.5555 | 1.3085 |
| A | 341 | SER | 61.1170 | 4.9569 | 56.1601 |
| A | 342 | ARG | 65.4430 | 15.2049 | 50.2381 |
| A | 343 | PRO | 7.4718 | 5.8928 | 1.5790 |
| A | 344 | SER | 46.7157 | 8.2264 | 38.4893 |
| A | 345 | PRO | 8.9624 | 0.1428 | 8.8196 |
| A | 346 | PHE | 54.3420 | 0.0000 | 54.3420 |
| A | 347 | ASP | 31.5363 | 0.0000 | 31.5363 |
| A | 348 | LEU | 7.5406 | 3.0628 | 4.4778 |
| A | 349 | PHE | 47.0594 | 17.8090 | 29.2504 |
| A | 350 | ILE | 65.6199 | 9.9606 | 55.6593 |
| A | 351 | ARG | 137.9028 | 24.7368 | 113.1660 |
| A | 352 | LYS | 142.0125 | 20.1726 | 121.8399 |
| A | 353 | SER | 48.9241 | 7.6848 | 41.2393 |
| A | 354 | PRO | 2.7838 | 1.3055 | 1.4783 |
| A | 355 | THR | 40.4021 | 4.8060 | 35.5961 |
| A | 356 | ILE | 0.7303 | 0.7288 | 0.0015 |
| A | 357 | THR | 38.1965 | 1.6685 | 36.5281 |
| A | 358 | CYS | 0.0000 | 0.0000 | 0.0000 |
| A | 359 | LEU | 57.2918 | 0.0000 | 57.2918 |
| A | 360 | VAL | 1.4811 | 0.0000 | 1.4811 |
| A | 361 | VAL | 26.4275 | 0.0000 | 26.4275 |
| A | 362 | ASP | 1.6559 | 0.0000 | 1.6559 |
| A | 363 | LEU | 119.6712 | 9.2033 | 110.4678 |
| A | 364 | ALA | 78.7333 | 14.3696 | 64.3637 |
| A | 365 | PRO | 79.3853 | 32.3651 | 47.0202 |
| A | 366 | SER | 40.2168 | 14.0242 | 26.1926 |
| A | 367 | LYS | 212.4325 | 43.2686 | 169.1639 |
| A | 368 | GLY | 39.8856 | 39.8856 | 0.0000 |
| A | 369 | THR | 102.7635 | 12.0075 | 90.7560 |
| A | 370 | VAL | 5.8020 | 1.2971 | 4.5049 |
| A | 371 | ASN | 70.8195 | 0.5930 | 70.2265 |
| A | 372 | LEU | 26.6375 | 25.3607 | 1.2768 |
| A | 373 | THR | 63.8640 | 3.8953 | 59.9687 |
| A | 374 | TRP | 19.5203 | 12.4611 | 7.0592 |
| A | 375 | SER | 49.4189 | 2.7649 | 46.6540 |
| A | 376 | ARG | 31.0928 | 7.7441 | 23.3486 |
| A | 377 | ALA | 77.5219 | 45.8060 | 31.7159 |
| A | 378 | SER | 66.6797 | 40.4771 | 26.2025 |
| A | 379 | GLY | 65.8988 | 65.8988 | 0.0000 |
| A | 380 | LYS | 116.4270 | 8.7936 | 107.6334 |
| A | 381 | PRO | 108.8295 | 14.6070 | 94.2226 |
| A | 382 | VAL | 54.2349 | 26.8569 | 27.3780 |
| A | 383 | ASN | 86.4753 | 13.4514 | 73.0239 |
| A | 384 | HIS | 173.6417 | 12.7135 | 160.9282 |
| A | 385 | SER | 51.7955 | 28.3694 | 23.4261 |
| A | 386 | THR | 72.2038 | 3.6585 | 68.5453 |
| A | 387 | ARG | 97.7388 | 26.1865 | 71.5523 |
| A | 388 | LYS | 115.4839 | 6.8986 | 108.5853 |
| A | 389 | GLU | 113.2234 | 35.2996 | 77.9238 |
| A | 390 | GLU | 116.4752 | 6.3676 | 110.1076 |
| A | 391 | LYS | 144.4826 | 24.7116 | 119.7710 |
| A | 392 | GLN | 113.9451 | 7.7214 | 106.2237 |
| A | 393 | ARG | 217.0740 | 36.0558 | 181.0182 |
| A | 394 | ASN | 127.4447 | 33.3744 | 94.0703 |
| A | 395 | GLY | 23.5351 | 23.5351 | 0.0000 |
| A | 396 | THR | 35.9405 | 2.2916 | 33.6489 |
| A | 397 | LEU | 37.3071 | 0.3897 | 36.9174 |
| A | 398 | THR | 36.6338 | 0.1084 | 36.5254 |
| A | 399 | VAL | 1.0056 | 0.0005 | 1.0051 |
| A | 400 | THR | 22.9397 | 0.0005 | 22.9392 |
| A | 401 | SER | 4.2325 | 0.0010 | 4.2315 |
| A | 402 | THR | 25.0142 | 0.7905 | 24.2237 |
| A | 403 | LEU | 1.2610 | 0.0000 | 1.2610 |
| A | 404 | PRO | 55.8249 | 3.3119 | 52.5130 |
| A | 405 | VAL | 10.7120 | 10.7120 | 0.0000 |
| A | 406 | GLY | 30.1703 | 30.1703 | 0.0000 |
| A | 407 | THR | 48.6461 | 5.0988 | 43.5474 |
| A | 408 | ARG | 142.5092 | 1.0113 | 141.4979 |
| A | 409 | ASP | 56.6478 | 3.0794 | 53.5684 |
| A | 410 | TRP | 1.0792 | 0.0006 | 1.0786 |
| A | 411 | ILE | 66.2093 | 21.0455 | 45.1638 |
| A | 412 | GLU | 145.4881 | 39.5895 | 105.8986 |
| A | 413 | GLY | 38.6895 | 38.6895 | 0.0000 |
| A | 414 | GLU | 22.1923 | 9.2475 | 12.9448 |
| A | 415 | THR | 40.6026 | 0.2438 | 40.3588 |
| A | 416 | TYR | 5.1230 | 0.0308 | 5.0922 |
| A | 417 | GLN | 62.3280 | 0.4057 | 61.9222 |
| A | 418 | CYS | 0.0000 | 0.0000 | 0.0000 |
| A | 419 | ARG | 103.9206 | 0.0000 | 103.9206 |
| A | 420 | VAL | 2.3852 | 1.4107 | 0.9744 |
| A | 421 | THR | 46.0180 | 1.1126 | 44.9053 |
| A | 422 | HIS | 16.6307 | 8.9744 | 7.6562 |
| A | 423 | PRO | 63.0244 | 42.0681 | 20.9562 |
| A | 424 | HIS | 126.3997 | 30.4096 | 95.9901 |
| A | 425 | LEU | 73.4692 | 13.0120 | 60.4572 |
| A | 426 | PRO | 136.4835 | 43.7065 | 92.7770 |
| A | 427 | ARG | 174.1798 | 16.4322 | 157.7477 |
| A | 428 | ALA | 31.7676 | 12.1334 | 19.6342 |
| A | 429 | LEU | 47.6693 | 3.3487 | 44.3206 |
| A | 430 | MET | 81.9649 | 21.6349 | 60.3301 |
| A | 431 | ARG | 102.1664 | 3.7496 | 98.4168 |
| A | 432 | SER | 74.7559 | 27.6174 | 47.1385 |
| A | 433 | THR | 12.0200 | 7.4697 | 4.5502 |
| A | 434 | THR | 54.0747 | 7.5188 | 46.5559 |
| A | 435 | LYS | 39.4746 | 13.2184 | 26.2562 |
| A | 436 | THR | 54.0546 | 6.8840 | 47.1706 |
| A | 437 | SER | 92.5356 | 13.8146 | 78.7209 |
| A | 438 | GLY | 44.5439 | 44.5439 | 0.0000 |
| A | 439 | PRO | 95.9599 | 12.1244 | 83.8355 |
| A | 440 | ARG | 85.9106 | 27.8327 | 58.0779 |
| A | 441 | ALA | 31.0495 | 7.8868 | 23.1627 |
| A | 442 | ALA | 52.0384 | 13.4854 | 38.5529 |
| A | 443 | PRO | 9.0934 | 9.0918 | 0.0015 |
| A | 444 | GLU | 50.5501 | 4.8813 | 45.6687 |
| A | 445 | VAL | 1.1996 | 1.1993 | 0.0004 |
| A | 446 | TYR | 33.1189 | 0.0000 | 33.1189 |
| A | 447 | ALA | 0.2103 | 0.2103 | 0.0000 |
| A | 448 | PHE | 6.4091 | 0.0021 | 6.4070 |
| A | 449 | ALA | 18.5169 | 13.0271 | 5.4898 |
| A | 450 | THR | 6.8652 | 1.0920 | 5.7732 |
| A | 451 | PRO | 85.0090 | 26.0777 | 58.9313 |
| A | 452 | GLU | 53.5564 | 21.4659 | 32.0906 |
| A | 453 | TRP | 233.7213 | 21.0784 | 212.6428 |
| A | 454 | PRO | 110.0223 | 22.9884 | 87.0339 |
| A | 455 | GLY | 58.2985 | 58.2985 | 0.0000 |
| A | 456 | SER | 83.8465 | 35.4457 | 48.4007 |
| A | 457 | ARG | 204.5646 | 33.7345 | 170.8301 |
| A | 458 | ASP | 74.3533 | 35.5320 | 38.8213 |
| A | 459 | LYS | 90.8795 | 5.6494 | 85.2301 |
| A | 460 | ARG | 39.6337 | 0.3815 | 39.2522 |
| A | 461 | THR | 0.9440 | 0.0000 | 0.9440 |
| A | 462 | LEU | 0.0000 | 0.0000 | 0.0000 |
| A | 463 | ALA | 0.0022 | 0.0022 | 0.0000 |
| A | 464 | CYS | 0.0189 | 0.0189 | 0.0000 |
| A | 465 | LEU | 0.0024 | 0.0000 | 0.0024 |
| A | 466 | ILE | 0.0969 | 0.0000 | 0.0969 |
| A | 467 | GLN | 4.2986 | 0.0000 | 4.2986 |
| A | 468 | ASN | 45.2933 | 4.2260 | 41.0673 |
| A | 469 | PHE | 0.0016 | 0.0000 | 0.0016 |
| A | 470 | MET | 45.6193 | 0.0006 | 45.6187 |
| A | 471 | PRO | 10.5390 | 4.5735 | 5.9655 |
| A | 472 | GLU | 66.1010 | 1.3044 | 64.7966 |
| A | 473 | ASP | 34.3329 | 6.6650 | 27.6679 |
| A | 474 | ILE | 28.1949 | 24.7847 | 3.4102 |
| A | 475 | SER | 15.8011 | 5.8009 | 10.0002 |
| A | 476 | VAL | 17.7598 | 14.8483 | 2.9115 |
| A | 477 | GLN | 8.8153 | 0.0008 | 8.8145 |
| A | 478 | TRP | 1.6124 | 0.9129 | 0.6996 |
| A | 479 | LEU | 3.6886 | 0.0000 | 3.6886 |
| A | 480 | HIS | 49.2343 | 0.4256 | 48.8086 |
| A | 481 | ASN | 60.9710 | 12.4862 | 48.4849 |
| A | 482 | GLU | 173.7820 | 37.6217 | 136.1603 |
| A | 483 | VAL | 89.3318 | 4.1549 | 85.1769 |
| A | 484 | GLN | 79.3073 | 21.3545 | 57.9528 |
| A | 485 | LEU | 27.3587 | 5.2803 | 22.0784 |
| A | 486 | PRO | 88.6492 | 13.2533 | 75.3958 |
| A | 487 | ASP | 99.0020 | 6.5842 | 92.4178 |
| A | 488 | ALA | 91.2923 | 30.7006 | 60.5917 |
| A | 489 | ARG | 85.3735 | 17.3474 | 68.0262 |
| A | 490 | HIS | 27.1818 | 17.0174 | 10.1644 |
| A | 491 | SER | 25.4648 | 5.3465 | 20.1183 |
| A | 492 | THR | 37.6322 | 11.4738 | 26.1583 |
| A | 493 | THR | 6.2876 | 3.0406 | 3.2469 |
| A | 494 | GLN | 120.0995 | 2.7999 | 117.2995 |
| A | 495 | PRO | 41.3128 | 17.2964 | 24.0164 |
| A | 496 | ARG | 67.3088 | 3.7186 | 63.5902 |
| A | 497 | LYS | 150.2799 | 14.2135 | 136.0663 |
| A | 498 | THR | 38.1648 | 34.4749 | 3.6899 |
| A | 499 | LYS | 148.5493 | 8.5618 | 139.9875 |
| A | 500 | GLY | 76.9764 | 76.9764 | 0.0000 |
| A | 501 | SER | 35.3537 | 27.8013 | 7.5524 |
| A | 502 | GLY | 8.6018 | 8.6018 | 0.0000 |
| A | 503 | PHE | 31.7527 | 1.3561 | 30.3966 |
| A | 504 | PHE | 0.8997 | 0.1297 | 0.7700 |
| A | 505 | VAL | 1.3819 | 0.0043 | 1.3776 |
| A | 506 | PHE | 0.0393 | 0.0393 | 0.0000 |
| A | 507 | SER | 0.0697 | 0.0697 | 0.0000 |
| A | 508 | ARG | 7.6439 | 2.8043 | 4.8396 |
| A | 509 | LEU | 0.3316 | 0.0344 | 0.2972 |
| A | 510 | GLU | 22.5879 | 7.4305 | 15.1574 |
| A | 511 | VAL | 7.0471 | 7.0471 | 0.0000 |
| A | 512 | THR | 80.5746 | 7.4380 | 73.1367 |
| A | 513 | ARG | 111.1689 | 3.1148 | 108.0541 |
| A | 514 | ALA | 58.1366 | 5.4416 | 52.6950 |
| A | 515 | GLU | 27.2188 | 0.1935 | 27.0253 |
| A | 516 | TRP | 44.3047 | 2.1698 | 42.1350 |
| A | 517 | GLU | 113.2078 | 31.9303 | 81.2775 |
| A | 518 | GLN | 44.8077 | 1.3672 | 43.4405 |
| A | 519 | LYS | 31.0147 | 0.0000 | 31.0147 |
| A | 520 | ASP | 84.0782 | 14.7455 | 69.3327 |
| A | 521 | GLU | 67.8496 | 3.1378 | 64.7117 |
| A | 522 | PHE | 2.3708 | 0.0000 | 2.3708 |
| A | 523 | ILE | 19.4152 | 0.0000 | 19.4152 |
| A | 524 | CYS | 0.0000 | 0.0000 | 0.0000 |
| A | 525 | ARG | 53.8480 | 0.0000 | 53.8480 |
| A | 526 | ALA | 0.1543 | 0.1521 | 0.0022 |
| A | 527 | VAL | 0.4091 | 0.0000 | 0.4091 |
| A | 528 | HIS | 0.9368 | 0.0000 | 0.9368 |
| A | 529 | GLU | 49.8255 | 6.7170 | 43.1084 |
| A | 530 | ALA | 25.3085 | 22.0423 | 3.2662 |
| A | 531 | ALA | 3.8937 | 3.7960 | 0.0977 |
| A | 532 | SER | 93.9367 | 25.5989 | 68.3378 |
| A | 533 | PRO | 113.4963 | 7.9201 | 105.5762 |
| A | 534 | SER | 69.1432 | 17.4384 | 51.7047 |
| A | 535 | GLN | 39.0154 | 0.0000 | 39.0154 |
| A | 536 | THR | 47.4246 | 15.9885 | 31.4361 |
| A | 537 | VAL | 37.3796 | 5.2662 | 32.1134 |
| A | 538 | GLN | 80.4137 | 17.3329 | 63.0807 |
| A | 539 | ARG | 114.1401 | 5.3396 | 108.8005 |
| A | 540 | ALA | 46.1789 | 7.2011 | 38.9778 |
| A | 541 | VAL | 7.4375 | 7.4375 | 0.0000 |
| A | 542 | SER | 53.4887 | 16.3308 | 37.1580 |
| A | 543 | VAL | 143.3700 | 54.8988 | 88.4713 |
| B | 336 | VAL | 63.0733 | 35.0684 | 28.0049 |
| B | 337 | SER | 40.7513 | 3.7350 | 37.0163 |
| B | 338 | ALA | 8.2164 | 8.2164 | 0.0000 |
| B | 339 | TYR | 124.9616 | 4.9609 | 120.0006 |
| B | 340 | LEU | 32.2736 | 30.2378 | 2.0358 |
| B | 341 | SER | 59.0456 | 4.4001 | 54.6455 |
| B | 342 | ARG | 68.6573 | 15.2123 | 53.4449 |
| B | 343 | PRO | 7.5644 | 6.0344 | 1.5300 |
| B | 344 | SER | 46.7394 | 8.2658 | 38.4735 |
| B | 345 | PRO | 8.8647 | 0.0661 | 8.7987 |
| B | 346 | PHE | 52.7081 | 0.0001 | 52.7079 |
| B | 347 | ASP | 32.0935 | 0.0000 | 32.0935 |
| B | 348 | LEU | 14.8371 | 3.0411 | 11.7960 |
| B | 349 | PHE | 49.2963 | 17.4349 | 31.8613 |
| B | 350 | ILE | 66.6755 | 11.3996 | 55.2758 |
| B | 351 | ARG | 138.6156 | 27.1648 | 111.4508 |
| B | 352 | LYS | 141.4297 | 19.8862 | 121.5435 |
| B | 353 | SER | 50.0186 | 7.0285 | 42.9901 |
| B | 354 | PRO | 2.2374 | 0.7342 | 1.5032 |
| B | 355 | THR | 41.7632 | 5.0415 | 36.7217 |
| B | 356 | ILE | 0.7365 | 0.7350 | 0.0014 |
| B | 357 | THR | 38.2632 | 1.6972 | 36.5659 |
| B | 358 | CYS | 0.0000 | 0.0000 | 0.0000 |
| B | 359 | LEU | 56.1126 | 0.0000 | 56.1126 |
| B | 360 | VAL | 1.5816 | 0.0000 | 1.5816 |
| B | 361 | VAL | 25.9197 | 0.0054 | 25.9143 |
| B | 362 | ASP | 1.8960 | 0.0000 | 1.8960 |
| B | 363 | LEU | 118.5662 | 9.4704 | 109.0958 |
| B | 364 | ALA | 77.0955 | 14.3989 | 62.6966 |
| B | 365 | PRO | 85.8755 | 32.6618 | 53.2137 |
| B | 366 | SER | 43.4697 | 13.7975 | 29.6721 |
| B | 367 | LYS | 206.9839 | 38.4032 | 168.5807 |
| B | 368 | GLY | 38.3029 | 38.3029 | 0.0000 |
| B | 369 | THR | 104.4643 | 11.7881 | 92.6762 |
| B | 370 | VAL | 5.3648 | 1.3354 | 4.0294 |
| B | 371 | ASN | 71.8454 | 0.6766 | 71.1688 |
| B | 372 | LEU | 27.2777 | 25.4561 | 1.8216 |
| B | 373 | THR | 63.4130 | 3.0688 | 60.3442 |
| B | 374 | TRP | 21.1747 | 14.5837 | 6.5910 |
| B | 375 | SER | 49.3968 | 2.8090 | 46.5877 |
| B | 376 | ARG | 33.5679 | 7.8441 | 25.7238 |
| B | 377 | ALA | 75.8852 | 45.5987 | 30.2865 |
| B | 378 | SER | 67.8229 | 40.7526 | 27.0703 |
| B | 379 | GLY | 66.3404 | 66.3404 | 0.0000 |
| B | 380 | LYS | 120.5164 | 9.2949 | 111.2215 |
| B | 381 | PRO | 109.6176 | 14.2385 | 95.3791 |
| B | 382 | VAL | 53.1169 | 25.2918 | 27.8251 |
| B | 383 | ASN | 91.8842 | 12.6656 | 79.2186 |
| B | 384 | HIS | 177.7631 | 13.1140 | 164.6491 |
| B | 385 | SER | 54.1445 | 30.9373 | 23.2073 |
| B | 386 | THR | 75.0451 | 3.9383 | 71.1068 |
| B | 387 | ARG | 103.3354 | 27.2889 | 76.0466 |
| B | 388 | LYS | 118.3613 | 6.6694 | 111.6919 |
| B | 389 | GLU | 102.2353 | 27.4529 | 74.7824 |
| B | 390 | GLU | 111.8330 | 2.2529 | 109.5801 |
| B | 391 | LYS | 164.3363 | 16.4358 | 147.9005 |
| B | 392 | GLN | 115.7355 | 9.9426 | 105.7929 |
| B | 393 | ARG | 243.1959 | 43.7367 | 199.4592 |
| B | 394 | ASN | 128.7879 | 34.6864 | 94.1016 |
| B | 395 | GLY | 24.2780 | 24.2780 | 0.0000 |
| B | 396 | THR | 36.3774 | 3.2281 | 33.1493 |
| B | 397 | LEU | 41.3144 | 1.2301 | 40.0844 |
| B | 398 | THR | 36.2084 | 0.1877 | 36.0207 |
| B | 399 | VAL | 0.9349 | 0.0000 | 0.9349 |
| B | 400 | THR | 22.7308 | 0.0000 | 22.7308 |
| B | 401 | SER | 4.4619 | 0.0000 | 4.4619 |
| B | 402 | THR | 26.2017 | 0.7838 | 25.4179 |
| B | 403 | LEU | 1.0199 | 0.0000 | 1.0199 |
| B | 404 | PRO | 56.9154 | 3.8109 | 53.1046 |
| B | 405 | VAL | 12.3109 | 12.3109 | 0.0000 |
| B | 406 | GLY | 29.3614 | 29.3614 | 0.0000 |
| B | 407 | THR | 42.7403 | 3.8469 | 38.8934 |
| B | 408 | ARG | 157.6823 | 3.1126 | 154.5697 |
| B | 409 | ASP | 51.4385 | 3.0432 | 48.3953 |
| B | 410 | TRP | 9.3345 | 0.0000 | 9.3345 |
| B | 411 | ILE | 95.1467 | 19.7743 | 75.3724 |
| B | 412 | GLU | 125.4698 | 37.7238 | 87.7460 |
| B | 413 | GLY | 18.7812 | 18.7812 | 0.0000 |
| B | 414 | GLU | 22.6140 | 9.6511 | 12.9629 |
| B | 415 | THR | 39.5185 | 0.3206 | 39.1980 |
| B | 416 | TYR | 5.9881 | 0.0409 | 5.9472 |
| B | 417 | GLN | 62.6966 | 0.3477 | 62.3489 |
| B | 418 | CYS | 0.0000 | 0.0000 | 0.0000 |
| B | 419 | ARG | 102.6084 | 0.0000 | 102.6084 |
| B | 420 | VAL | 2.7707 | 1.6846 | 1.0861 |
| B | 421 | THR | 46.0095 | 0.8195 | 45.1900 |
| B | 422 | HIS | 16.7868 | 8.9411 | 7.8457 |
| B | 423 | PRO | 62.8378 | 41.9162 | 20.9217 |
| B | 424 | HIS | 125.3588 | 30.3775 | 94.9814 |
| B | 425 | LEU | 71.5870 | 12.6261 | 58.9609 |
| B | 426 | PRO | 136.6526 | 45.8124 | 90.8402 |
| B | 427 | ARG | 170.2552 | 16.6453 | 153.6099 |
| B | 428 | ALA | 32.0708 | 12.4769 | 19.5939 |
| B | 429 | LEU | 48.8898 | 2.1566 | 46.7332 |
| B | 430 | MET | 84.2805 | 22.5773 | 61.7032 |
| B | 431 | ARG | 100.5128 | 3.0888 | 97.4240 |
| B | 432 | SER | 75.1133 | 26.0086 | 49.1047 |
| B | 433 | THR | 10.4817 | 6.7238 | 3.7580 |
| B | 434 | THR | 48.7159 | 5.3780 | 43.3380 |
| B | 435 | LYS | 72.0172 | 7.7237 | 64.2934 |
| B | 436 | THR | 58.8140 | 9.6274 | 49.1866 |
| B | 437 | SER | 83.7729 | 12.9694 | 70.8035 |
| B | 438 | GLY | 44.1275 | 44.1275 | 0.0000 |
| B | 439 | PRO | 98.8394 | 12.2219 | 86.6175 |
| B | 440 | ARG | 88.9167 | 29.3709 | 59.5458 |
| B | 441 | ALA | 32.3694 | 7.8101 | 24.5593 |
| B | 442 | ALA | 52.2561 | 13.8471 | 38.4090 |
| B | 443 | PRO | 9.1527 | 9.0687 | 0.0839 |
| B | 444 | GLU | 77.6629 | 1.5591 | 76.1038 |
| B | 445 | VAL | 2.0960 | 2.0960 | 0.0000 |
| B | 446 | TYR | 36.2935 | 0.0000 | 36.2935 |
| B | 447 | ALA | 0.1863 | 0.1863 | 0.0000 |
| B | 448 | PHE | 6.5491 | 0.0000 | 6.5491 |
| B | 449 | ALA | 18.6090 | 12.5251 | 6.0838 |
| B | 450 | THR | 4.4299 | 1.1588 | 3.2711 |
| B | 451 | PRO | 76.6290 | 22.7457 | 53.8833 |
| B | 452 | GLU | 55.5072 | 22.2581 | 33.2491 |
| B | 453 | TRP | 235.9264 | 21.1124 | 214.8139 |
| B | 454 | PRO | 111.4380 | 22.8008 | 88.6373 |
| B | 455 | GLY | 74.9059 | 74.9059 | 0.0000 |
| B | 456 | SER | 92.2118 | 45.4910 | 46.7208 |
| B | 457 | ARG | 219.5241 | 23.6314 | 195.8927 |
| B | 458 | ASP | 71.4157 | 34.5621 | 36.8536 |
| B | 459 | LYS | 95.3227 | 8.3619 | 86.9608 |
| B | 460 | ARG | 40.1231 | 0.1393 | 39.9838 |
| B | 461 | THR | 1.4382 | 0.0000 | 1.4382 |
| B | 462 | LEU | 0.0000 | 0.0000 | 0.0000 |
| B | 463 | ALA | 0.0000 | 0.0000 | 0.0000 |
| B | 464 | CYS | 0.0199 | 0.0199 | 0.0000 |
| B | 465 | LEU | 0.7613 | 0.4703 | 0.2910 |
| B | 466 | ILE | 0.1384 | 0.0000 | 0.1384 |
| B | 467 | GLN | 6.3987 | 0.0000 | 6.3987 |
| B | 468 | ASN | 44.9702 | 3.7128 | 41.2575 |
| B | 469 | PHE | 0.0026 | 0.0000 | 0.0026 |
| B | 470 | MET | 46.3764 | 0.0005 | 46.3759 |
| B | 471 | PRO | 15.0525 | 5.0794 | 9.9731 |
| B | 472 | GLU | 65.5924 | 1.4428 | 64.1496 |
| B | 473 | ASP | 46.5992 | 6.7495 | 39.8497 |
| B | 474 | ILE | 28.5269 | 25.0290 | 3.4979 |
| B | 475 | SER | 15.9668 | 5.7847 | 10.1821 |
| B | 476 | VAL | 17.4473 | 14.3713 | 3.0760 |
| B | 477 | GLN | 9.3877 | 0.0000 | 9.3877 |
| B | 478 | TRP | 1.3921 | 0.8722 | 0.5198 |
| B | 479 | LEU | 4.3690 | 0.0000 | 4.3690 |
| B | 480 | HIS | 54.2843 | 0.6477 | 53.6366 |
| B | 481 | ASN | 58.4959 | 9.8012 | 48.6946 |
| B | 482 | GLU | 167.3206 | 33.4531 | 133.8675 |
| B | 483 | VAL | 89.2539 | 4.2127 | 85.0413 |
| B | 484 | GLN | 76.3506 | 21.2575 | 55.0931 |
| B | 485 | LEU | 25.8432 | 4.5546 | 21.2886 |
| B | 486 | PRO | 85.9602 | 12.9613 | 72.9989 |
| B | 487 | ASP | 101.5779 | 6.8540 | 94.7239 |
| B | 488 | ALA | 89.7437 | 31.7759 | 57.9678 |
| B | 489 | ARG | 83.4391 | 17.2390 | 66.2001 |
| B | 490 | HIS | 27.4157 | 17.3316 | 10.0841 |
| B | 491 | SER | 24.9127 | 5.0237 | 19.8890 |
| B | 492 | THR | 37.6675 | 11.4176 | 26.2499 |
| B | 493 | THR | 5.6950 | 2.6368 | 3.0582 |
| B | 494 | GLN | 117.7065 | 3.0565 | 114.6500 |
| B | 495 | PRO | 40.3432 | 17.1857 | 23.1575 |
| B | 496 | ARG | 70.2823 | 3.8397 | 66.4426 |
| B | 497 | LYS | 150.2087 | 14.1413 | 136.0674 |
| B | 498 | THR | 37.9054 | 34.1687 | 3.7367 |
| B | 499 | LYS | 148.9955 | 8.7157 | 140.2798 |
| B | 500 | GLY | 79.0145 | 79.0145 | 0.0000 |
| B | 501 | SER | 35.5772 | 28.0187 | 7.5585 |
| B | 502 | GLY | 8.7428 | 8.7428 | 0.0000 |
| B | 503 | PHE | 31.0098 | 0.6811 | 30.3286 |
| B | 504 | PHE | 0.8456 | 0.1156 | 0.7300 |
| B | 505 | VAL | 1.3055 | 0.0259 | 1.2796 |
| B | 506 | PHE | 0.0679 | 0.0679 | 0.0000 |
| B | 507 | SER | 0.1087 | 0.0000 | 0.1087 |
| B | 508 | ARG | 7.6625 | 2.7772 | 4.8853 |
| B | 509 | LEU | 0.2769 | 0.0638 | 0.2131 |
| B | 510 | GLU | 23.9603 | 7.2457 | 16.7146 |
| B | 511 | VAL | 7.2569 | 7.2569 | 0.0000 |
| B | 512 | THR | 80.3374 | 7.5753 | 72.7621 |
| B | 513 | ARG | 111.7661 | 3.0775 | 108.6887 |
| B | 514 | ALA | 68.3844 | 10.3023 | 58.0821 |
| B | 515 | GLU | 28.0062 | 5.9456 | 22.0606 |
| B | 516 | TRP | 44.6731 | 2.2532 | 42.4200 |
| B | 517 | GLU | 101.0924 | 22.5848 | 78.5077 |
| B | 518 | GLN | 79.1739 | 0.0000 | 79.1739 |
| B | 519 | LYS | 30.5550 | 0.0000 | 30.5550 |
| B | 520 | ASP | 81.7324 | 14.5855 | 67.1468 |
| B | 521 | GLU | 51.8306 | 4.1150 | 47.7156 |
| B | 522 | PHE | 1.2433 | 0.0000 | 1.2433 |
| B | 523 | ILE | 27.5515 | 0.0013 | 27.5502 |
| B | 524 | CYS | 0.0000 | 0.0000 | 0.0000 |
| B | 525 | ARG | 53.0495 | 0.0000 | 53.0495 |
| B | 526 | ALA | 0.0470 | 0.0470 | 0.0000 |
| B | 527 | VAL | 0.5077 | 0.0000 | 0.5077 |
| B | 528 | HIS | 0.8503 | 0.0026 | 0.8477 |
| B | 529 | GLU | 70.9722 | 6.7364 | 64.2357 |
| B | 530 | ALA | 24.8160 | 21.6506 | 3.1654 |
| B | 531 | ALA | 3.8910 | 3.8714 | 0.0196 |
| B | 532 | SER | 93.3813 | 26.2679 | 67.1135 |
| B | 533 | PRO | 112.7473 | 7.5061 | 105.2412 |
| B | 534 | SER | 69.2111 | 17.8241 | 51.3871 |
| B | 535 | GLN | 38.1066 | 0.0000 | 38.1066 |
| B | 536 | THR | 43.5047 | 15.6901 | 27.8146 |
| B | 537 | VAL | 37.1147 | 5.1494 | 31.9653 |
| B | 538 | GLN | 75.6644 | 17.3404 | 58.3241 |
| B | 539 | ARG | 106.7137 | 5.0454 | 101.6683 |
| B | 540 | ALA | 44.2412 | 8.0048 | 36.2363 |
| B | 541 | VAL | 7.5264 | 7.5264 | 0.0000 |
| B | 542 | SER | 52.7855 | 16.1622 | 36.6233 |
| B | 543 | VAL | 145.3042 | 56.7124 | 88.5918 |

Residues that are solvent accessible are important as they represent amino acids that are on the external surface of the protein and, as such, may be involved in binding of a FcR to an antibody and be useful in designing proteins with an enhanced binding activity or in identifying compounds that inhibit such binding. In addition, solvent accessible residues can represent targets for modification to produce a Fc region of an antibody with improved function. Such analysis also identifies residues in the interior, or core, of the proteins in the complex. Such residues can also be targeted to produce proteins with improved functions, such as enhanced stability.

A model of the present invention also provides additional information that is not available from other sources. For example, a model can identify the crystal contacts between crystals and predict the location of the FcR binding domain, including those amino acids that actually form contacts with a FcR. Particularly important regions of the model representing the coordinates of Table 1, Table 2 or Table 3 include, but are not limited to the interdomain groove (i.e., space, gap) between the two Cε3/Cε4-containing chains of the IgE antibody Fc region, the hinge between the Cε3 and Cε4 domains of the Fc region, and a loop involved in FcεRIα protein binding, such as the linker between Cε2 and Cε3, the BC loop of Cε3, the DE loop of Cε3, and the FG loop of Cε3. These sites are described in more detail in the Examples and represent sites to target for drug design and mutein production.

A model of the present invention can also represent a complex that includes a Fc domain of an antibody that binds to a FcεRIα protein with an affinity that is at least equivalent to the affinity of a human IgE antibody Fc-Cε3/Cε4 region for the extracellular domain of any of the following FcεRIα proteins: a human FcεRIα protein, a canine FcεRIα protein, a feline FcεRIα protein, an equine FcεRIα protein, a murine FcεRIα protein and a rat FcεRIα protein. Such a model can represent a FceRI-binding domain of a human, canine, feline, equine, murine or rat Fc region. Such a model can also represent a Fc region with altered substrate specificity, preferably designed based on a model of the present invention.

The present invention includes a model that represents a Fc domain that binds to a Fc receptor of its respective class. Also included is a model that represents a Fc region of an antibody designed to bind to a Fc receptor of a class other than the class to which the protein naturally binds. Such classes include IgA, IgD, IgE, IgG, and IgM. Such a model of the present invention can be produced, for example, by incorporating all or any part of the amino acid sequence of the other antibody into a 3-D model substantially representing the coordinates in Table 1, Table 2 or Table 3. Such an embodiment includes any model that specifically incorporates any Ig domains that are placed in an orientation (packing interfaces and bend angles) that is based on the structure predicted by the coordinates in Table 1, Table 2 or Table 3 or a similar structure such that the distance between the two antibody-binding domains (i.e., Cε3 for IgE) ranges from about 10 angstroms to about 25 angstroms or from about 20 angstroms to about 40 angstroms. As such, both open and closed conformations of Fc regions are included in the present invention. In one embodiment, a model of the present invention is a 3-D model of a FcεRIα binding domain other than a human FcεRIα binding domain. Such proteins and models thereof can be designed by homology modeling.

A preferred modified model of the present invention is a model that has a 3-D structure comprising atomic coordinates that have a root mean square deviation of protein backbone atoms of less than 10 angstrom when superimposed, using backbone atoms, on the 3-D model substantially represented by the atomic coordinates specified in Table 1, Table 2 or Table 3. Preferably such a model has a 3-D structure comprising atomic coordinates that have a root mean square deviation of protein backbone atoms of less than 8 angstroms, preferably less than 7 angstroms, preferably less than 6 angstroms, preferably less than 5 angstroms, preferably less than 4 angstroms, preferably less than 3 angstroms, preferably less than 2 angstroms, and preferably less than I angstroms, when superimposed, using backbone atoms, on the 3-D model substantially represented by the atomic coordinates specified in Table 1, Table 2 or Table 3. In this embodiment, such a model represents a Fc region binding to a FcR. The backbone atoms are those atoms that form the backbone, or 3-D folding pattern, of the model. As such, backbone atoms are the base residues of amino acids, i.e., nitrogen, carbon, the alpha carbon and oxygen. Also preferred is a model modification that includes a Fc region having an amino acid sequence that shares at least about 30%, preferably at least about 40%, more preferably at least about 45%, more preferably at least about 50%, more preferably at least about 60% and even more preferably at least about 80% amino acid sequence homology, with a Fc-Cε3/Cε4 region of a human IgE antibody, as determined using the program ALIGN with default parameters, optimal global alignment of two sequences with no short-cuts. A preferred model of the present invention represents a FcεRIα-binding domain, i.e., a region that binds to a FcεRIα protein.

One embodiment of the present invention is a 3-D model of a human Fc-Cε3/Cε4 region produced by a method that includes the steps of: (a) crystallizing a human Fc-Cε3/Cε4 region, such as, but not limited to a protein having amino acid sequence SEQ ID NO:2; (b) collecting X-ray diffraction data from the crystallized protein; and (c) determining the model from the X-ray diffraction data, preferably in combination with an amino acid sequence of the protein. A complex for crystal formation can be produced using a variety of techniques well known to those skilled in the art. As disclosed herein, a human Fc-Cε3/Cε4 region to be crystallized is preferably produced in recombinant insect cells transformed with a gene encoding such a region, such as a baculovirus genetically engineered to produce the respective protein. The purity of the Fc-Cε3/Cε4 region must be sufficient to permit the production of crystals that can be analyzed by X-ray crystallography to a resolution that permits determination of a 3-D model of the protein. Preferably the resolution is at least about 4.5 angstroms (i.e., 4.4 angstroms or better), more preferably at least about 4 angstroms, more preferably at least about 3.5 angstroms, more preferably at least about 3.25 angstroms, more preferably at least about 3 angstroms, more preferably at least about 2.5 angstroms, more preferably at least about 2.3 angstroms, more preferably at least about 2 angstroms and even more preferably at least about 1.5 angstroms. Methods to obtain such purity levels are well known to those skilled in the art.

As disclosed herein, a preferred method to crystallize a Fc-Cε3/Cε4 region is by vapor distillation. Particularly preferred methods are disclosed in the Examples. It should be appreciated that the present invention also includes other methods known to those skilled in the art by which such a complex can be crystallized.

3-D models of some proteins have been determined; see, for example, Blundell et al., Protein Crystallography, Academic Press, London, 1976. However, as discussed herein, elucidation of the crystal structure of a Fc-Cε3/Cε4 region of a human IgE was difficult. In one embodiment, crystal structure determination includes obtaining high-resolution data using synchrotron radiation. Such data can be collected, for example, at the Stanford Synchrotron Source Laboratory, Palo Alto, Calif., or the Advanced Photon Source at Argonne National Laboratories, Argonne, Ill. Additional locations to collect such data include, but are not limited to, Brookhaven, N.Y., and Japan. In one embodiment, diffraction data from native and heavy-atom treated crystals provide an initial image of the protein structure which is refined into an electron density map. Details regarding data collection and interpretation are provided in the Examples section.

One embodiment of the present invention is a method to produce a 3-D model of a Fc region that includes positioning amino acid representations (i.e., representing amino acids) of the protein at substantially the coordinates listed in Table 1, Table 2 or Table 3. That is, knowledge of the coordinates of the complex permits one skilled in the art to produce a model of the protein using those coordinates. Such a model, or any model which is essentially represented by a simple coordinate transformation of the coordinates specified in Table 1, Table 2 or Table 3, can be represented in a variety of methods as heretofore disclosed and is included in the present invention.

In another embodiment, a model of the present invention can be refined to obtain an improved model, which is an example of a model modification, also referred to as a modified model. Refining methods can include, but are not limited to, further data collection and analysis; data collection from frozen crystals; introduction of solvent molecules to the structure; clarification of secondary structure; and analyses of crystallized complexes between a FcR and an antibody or inhibitory compound or of crystallized FcRs or antibodies alone. An additional model refinement method includes analyzing a 3-D model to predict amino acid residues that if replaced are likely to yield proteins with at least one improved function, effecting at least one such replacement, determining whether the activity of the modified protein agrees with the prediction, and refining the model as necessary. Methods to determine whether the modification agrees with prediction include producing the modified protein and performing assays with that modified protein to determine if the protein does indeed exhibit the improved function(s), such as desired activity, stability and solubility properties. Assays to measure such functions are well known in the art; examples of several such assays are disclosed herein.

Another embodiment of the present invention is a modified 3-D model that represents an antibody other than human IgE as represented by the coordinates in Table 1, Table 2 or Table 3. Preferably the amino acid sequence of the protein(s) to be modeled is known. In such a case, the modified model can be produced using the technique of homology modeling, preferably by incorporating (e.g., grafting, overlaying or replacing) all or any portion of the amino acid sequence of the other antibody into the 3-D model representing the coordinates of Table 1, Table 2 or Table 3 to produce the modified model. General techniques for homology modeling, also referred to as molecular replacement, have been disclosed in, for example, Greer, 1990, Proteins: Structure, Function, and Genetics 7, 317-334; Havel et al., 1991, J. Mol. Biol. 217, 1-7; Schiffer et al., 1990, Proteins: Structure, Function, and Genetics 8, 30-43; and Lattman, 1985, Methods Enzymol 115, 55-77. However, such technology has not been applied to Fc regions of IgE antibodies since, until the present invention, no 3-D model of any Fc region of IgE was available. Thus, the present invention now allows the solving of the structures of a number of other natural and mutated forms of antibodies.

In one embodiment, a model of a Fc region, such as, but not limited to a Fc-Cε3/Cε4 region, is produced by extracting the 3-D coordinates from a published figure or building a 3-D model with atoms from other domains wherein FcR-binding domains of the antibody are oriented as predicted for a human Fc-Cε3/Cε4₂₂₂ protein. For example, a model of the present invention can be produced by orienting two known FcR-binding domains into a bent confirmation such that the distance between the domains ranges from about 10 to about 25 angstroms or from about 20 to about 40 angstroms. In another example, a model can be produced by orienting the hinge between two Ig domains in a manner similar to that specified by the coordinates in Table 1, Table 2 or Table 3. Such a model is referred to as a model in which the hinge between two Ig domains, e.g., between Cε3 and Cε4 are oriented in a manner as specified by the structural coordinates listed in Table 1, Table 2 or Table 3. This model can then be used in further molecular replacement methods. Such methods can include the steps of (a) orienting the model by three rotations; and (b) translating the model in one to three directions to produce additional model modifications.

Suitable antibodies for which a 3-D model can be determined using homology modeling include any mammalian antibody such as a protein that binds to a FcR for IgE, IgG, IgM, IgA or IgD antibodies. Preferred antibodies that bind to FcRs include human, canine, feline, equine, murine and rat antibodies. The present invention also includes the use of other Ig domains to produce models of the present invention.

One embodiment of the present invention is a 3-D model of a Fc region of an antibody in which the protein has an improved function compared to an unmodified protein as well as a method to produce such a modified model. Such an improved function includes, but is not limited to, enhanced activity, enhanced stability and enhanced solubility. Such a modified model can be produced by replacing at least one amino acid based on information derived from analyzing the 3-D model representing the coordinates in Table 1, Table 2 or Table 3, such that the replacement leads to a protein with an improved function. As used herein, a replacement refers to an (i.e., one or more) amino acid substitution, insertion, deletion, inversion and/or derivatization (e.g., acetylation, glycosylation, phosphorylation, PEG modification, biotinylation, and covalent attachment of other ligands or other compounds to the protein. In one embodiment, synthetic chemical methods are used to produce either a fragment or the entire protein to, for example, introduce non-natural amino acids or other chemical compounds into the structure of a Fc region. For example, based on a structure of the present invention, one can design synthetic peptides or larger proteins that could be linked to produce an intact protein with FcR binding activity, the structure allowing one to design the start and stop points for these peptides, e.g., at surface accessible loops. In accordance with the present invention, an amino acid that is substituted or inserted can be a natural amino acid or an unnatural amino acid, including a derivitized amino acid. Methods to identify regions in the protein that, if changed, yield a protein with an improved function are disclosed below.

The present invention includes use of a 3-D model of the present invention to identify a compound that inhibits binding between a FcR and an antibody. The advantages of using a 3-D model to identify inhibitory compounds are multi-fold in that the model depicts the site at which a Fc region of an antibody binds to its FcR, i.e., the antibody-binding domain, also referred to as the antibody binding site, and the FcR-binding domain, also referred to as the FcR binding site. The antibody binding site and the FcR binding site together form an FcR:antibody interaction site. As such, a large number of potential inhibitory compounds can be initially analyzed without having to perform in vitro or in vivo laboratory studies. As used herein, methods to identify inhibitory compounds include, but are not limited to, designing inhibitory compounds based on the 3-D model of a Fc region, probing such a 3-D model with compounds that are potential inhibitors in order to identify those compounds that are actually inhibitory of the binding of an antibody to its FcR, screening a compound data base using such a 3-D model to identify compounds that inhibit such binding, and combinations thereof. Methods to use 3-D models to design, probe for, or screen for suitable inhibitory compounds are known to those skilled in the art. In particular, there are a number of computer programs that enable such methods. See, for example, PCT Publication No. WO 95/35367, by Wilson et al., published Dec. 28, 1995, which is incorporated by reference herein in its entirety.

An inhibitory compound can be any natural or synthetic compound that inhibits the binding of an antibody to a FcR. Examples include, but are not limited to, inorganic compounds, oligonucleotides, proteins, peptides, antibodies, antibody fragments, mimetics of peptides or antibodies (such as, mimetics of antibody or receptor binding sites), and other organic compounds. Compounds can inhibit binding in either a competitive or non-competitive manner and can either interact at the binding site or allosterically. An inhibitory compound should be capable of physically and structurally associating with a FcR and/or an antibody such that the compound can inhibit binding between the two entitites. As such, an inhibitory compound is preferably small and is of a structure that effectively prevents or disrupts binding. Inhibitory compounds can be identified in one or multiple steps. For example, a compound initially identified that inhibits binding between an antibody and FcR to some extent can be used as a lead to design, probe or screen for a compound with improved characteristics, such as greater efficacy, safety, solubility, etc. A preferred inhibitory compound is a compound that is efficacious when administered to an animal in an amount that results in a serum concentration of from about 1 nanomolar (nM) to 100 micromolar (uM), with a concentration of from about 10 nM to 10 uM being more preferred.

One embodiment of the present invention is a method to identify a compound that inhibits the binding between an IgE antibody and a FcεRIα protein. Such a method includes the step of using a 3-D model substantially representing the atomic coordinates specified in Table 1, Table 2 or Table 3 to identify such a compound. Included in the present invention are inhibitory compounds that interact directly with the IgE binding domain or the receptor binding domain of the IgE antibody as well as compounds that interact indirectly with such structures. Preferably a compound interacts with at least one of the following regions: a FcεRIα binding domain, an interdomain groove between the two Cε3/Cε4 domains of the antibody Fc region, a hinge between Cε3 and Cε4 domains of the antibody Fc region, and a region of a Cε3 or Cε4 domain, the relative position of which changes by greater than 1 angstrom between closed and receptor-bound Fc-Cε3/Cε4 conformations. It is to be noted that many residues in the Cε3 domains are significantly closer to the Cε4 domains in the closed form of the IgE as compared to the open form. While not being bound by theory, it is believed that molecules that could interact with Cε3 residues and Cε4 residues at the same time but only in the closed form of the IgE, would be potential inhibitors. Regions to target include a set of residues in the two domains whose relative distances change significantly (i.e., by more than 1 angstrom) in comparison of the receptor-bound and closed IgE conformations. Preferably the distance between the two Cε3 domains of the closed conformation of the Fc-Cε3/Cε4 region ranges from about 10 to about 25 angstroms, more preferably from about 10 and 15 angstroms, and even more preferably about 13 angstroms. In a preferred embodiment, an inhibitory compound reacts with at least one of the following regions: a linker between Cε2 and Cε3 (amino acids 4, 7, 8, 9, 10 and 11 of SEQ ID NO:2); a BC loop of Cε3 (amino acids 37, 38 and 39 of SEQ ID NO:2; a DE loop of Cε3 (amino acids 68, 69, and 70 of SEQ ID NO:2); a FG loop of Cε3 (amino acids 99, 100, 101 and 102 of SEQ ID NO:2); a loop or strand defining (i.e., abutting, forming) the interdomain groove; a AB helix of Cε3 (amino acid 20, 21, 22, 23 and 24 of SEQ ID NO:2) which is thought to regulate the full conformational flexibility of the IgE-Fc region; and a region lying above said AB helix of Cε3, i.e., the region constituting the hinge and including amino acids 17, 18 and 19 (after strand A), amino acids 29, 30 and 31 (after strand B), and amino acids 109, 110 and 111 (after strand G) of SEQ ID NO:2. Particularly preferred amino acids with which to have an inhibitory compound interact include: (a) a residue at position 4, 7, 8, 9, 10, 11, 17, 18, 19, 20, 21, 22, 23, 24, 29, 30, 31, 37, 38, 39, 68, 69, 70, 99, 100, 101, 102, 109, 110, or 111 of SEQ ID NO:2; and (b) a surface residue within about 10 angstroms of any of said residues of (a). Even more preferred residues include: (a) a residue at position 4, 7, 8, 9, 10, 11, 37, 38, 39, 68, 69, 70, 99, 100, 101, or 102 of SEQ ID NO:2; (b) a residue in a region of a Cε3 or Cε4 domain, the relative position of which changes by greater than 1 angstrom between closed and receptor-bound Fc-Cε3/Cε4 conformations; and (c) a surface residue within about 10 angstroms of any of said residues of (a) or (b). Also preferred are additional residues identified in the Examples as being in at least one of the above cited regions. One preferred embodiment is a compound that inhibits the ability of an IgE antibody to convert from a closed conformation into a receptor-bound or open conformation. It is to be noted that the ability to identify such key regions and residues is only possible in view of a model of the present invention. In one embodiment, an inhibitory compound of the present invention is a peptide corresponding to at least a portion of any of the identified regions or a derivative thereof, such as a peptide mimetic or other compound that mimics that peptide.

One embodiment of a method to identify a compound that inhibits the binding between an IgE antibody and a FcεRIα protein includes the steps of: (a) generating a model substantially representing the atomic coordinates listed in Table 1, Table 2 or Table 3 or of the binding domains thereof, on a computer screen; (b) generating the spacial structure of a compound to be tested; and (c) testing to determine if the compound interacts with said FcR binding domain, wherein such an interaction indicates that the compound is capable of inhibiting the binding of an IgE antibody to a FcεRIα protein. In a preferred embodiment, step (a) includes the step of identifying one or more amino acid(s) in the FcR binding domain of the model that interact directly with the FcR. Preferably a compound to be tested will interact directly with one or more of those amino acid(s). Preferred amino acids with which an inhibitory compound should interact are disclosed herein.

The present invention also includes inhibitory compounds isolated in accordance with the methods disclosed herein. Methods to produce such compounds in quantities sufficient for use, for example, as protective agents (e.g., preventatives or therapeutics) are known to those skilled in the art. It should also be appreciated that it is within the scope of the present invention to expand the use of models of the present invention to produce models of any suitable Fc regions (i.e., model modifications) and to identify compounds that inhibit the binding of antibodies to such Fc regions.

A preferred inhibitory compound of the present invention, or lead that can be used to produce a more efficacious inhibitory compound, is a saturated tetracyclic hydrocarbon perhydrocyclopentanophenanthrene or a derivative thereof. Such a compound can include a structure having the following formula:

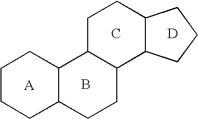

It is to be understood that such a compound can have any number of “R” groups, even though they are not indicated in the formula. Examples of saturated tetracyclic hydrocarbon perhydrocyclopentanophenanthrenes include, but are not limited to, isoprenoids, terpenes, bile acids, detergents (such as CHAPS and CHAPSO) cholestanes, cholic acids, cholesterols, androgens, estrogens, and other steroids. A preferred inhibitory compound, or compound to use as a lead to design a more efficacious compound is 3-[3-(cholamidopropyl) dimethylammonio]-1-propane-sulfonate (CHAPS) or a compound having a similar ring structure. The interaction of CHAPS with amino acids in the FcεRIα protein and Fc-Cε3/Cε4 region is described in further detail in Ser. No. 60/189,853, ibid.

In one embodiment, an inhibitory compound of the present invention is a bivalent, or other multivalent, compound that interacts with the two Cε3/Cε4 domains with high affinity or a compound that is sufficiently large to bind the interdomain groove, such as, but not limited to, macromolecules such as in vitro selected peptides, peptoids, nucleic acids, similar molecules, mimetopes thereof.

The present invention also includes use of a 3-D model of the present invention to rationally design and construct modified forms of Fc regions of antibodies, and particularly of IgE antibodies, that have one or more improved functions, such as, but not limited to, increased activity, increased stability and increased solubility compared to an unmodified Fc region of an IgE antibody. Muteins of the present invention include full-length proteins as well as fragments (i.e., truncated versions) of such proteins.

One embodiment of the present invention is a Fc region that comprises a mutein that binds to a Fc binding domain of a FcR. Such a mutein has an improved function compared to a protein comprising SEQ ID NO:2. Examples of such an improved function include, but are not limited to, increased stability, increased affinity for an FcR, altered substrate specificity, and increased solubility. Such a mutein can be produced by a method that includes the steps of: (a) analyzing a 3-D model substantially representing the atomic coordinates specified in Table 1, Table 2 or Table 3 to identify at least one amino acid of the protein represented by the model which if replaced by a specified amino acid would effect the improved function of the protein; and (b) replacing the identified amino acid(s) to produce a mutein having the improved function. Knowledge of the coordinates allows one to target specific residues, e.g. in the hydrophobic core or on the surface, to generate an accessible set of variants that can then be selected for a particular property, e.g. high stability, high affinity, altered substrate specificity, or other desirable properties (i.e., improved functions). Without the coordinates, one would have to analyze an extraordinarily large number of variants, e.g., on the order of ˜10¹¹ possibilities. The structure, in contrast, allows one to pick the most relevant residues for selecting a desired property by, for example, phage display or other methods. In a preferred embodiment, replacement of one or more amino acids does not substantially disrupt the 3-D structure of the protein; i.e., the modified protein, or mutein, is still capable of binding to the FcR. A preferred mutein is a Fc domain of an IgE antibody that binds to a FcεRIα protein, although the invention also covers muteins binding to other classes of FcRs.

In one embodiment, a mutein of the present invention has increased stability compared to its unmodified counterpart. As used herein, increased stability refers to the ability of a mutein to be more resistant, for example, to higher or lower temperature, to more acidic or basic pH, to higher or lower salt concentrations, to oxidation and/or reduction, to deamidation, to other forms of chemical degradation and to proteolytic degradation compared to an unmodified Fc region. Increased stability can also refer to the ability of a mutein of the present invention to be stable for a longer period of time either during storage (i.e., to have a longer shelf life) or during use (i.e., to have a longer half-life under reaction conditions) than does an unmodified protein. Muteins of the present invention can also exhibit a decreased entropy of unfolding, thereby stabilizing the proteins. Increased stability can be measured using a variety of methods known to those skilled in the art; examples include, but are not limited to, determination of melting temperature, thermal denaturation, pressure denaturation, enthalpy of unfolding, free energy of the protein, or stability in the presence of a chaotropic agents such as urea, guanidinium chloride, guanidinium thiocyanate, etc. A preferred mutein of the present invention has a melting temperature substantially higher than that of an unmodified Fc region. Preferably the melting temperature of a mutein is at least about 1° C. higher, and more preferably at least about 10° C. higher than the melting temperature of the corresponding unmodified protein. Also preferred is a mutein having binding activity over a pH range that is at least about 1 pH unit higher and/or lower than the active pH range of the corresponding unmodified protein.

Another embodiment of the present invention is a mutein that exhibits increased affinity for a FcR compared to its unmodified counterpart. As used herein, a mutein having increased affinity is a Fc region that exhibits a higher affinity constant (K_{A}) or lower dissociation constant (K_{D}) than its unmodified counterpart. Such a higher affinity constant can be achieved by increasing the association rate (kₐ) between the mutein and the FcR and/or decreasing the dissociation rate (k_{d}) between the mutein and the FcR. A preferred mutein of the present invention has a K_{A} for a FcR of at least about 3×10⁹ liters/mole (M⁻¹), which is equivalent to a K_{D} of less than or equal to about 3.3×10⁻¹⁰ moles/liter (M). More preferred is a mutein having a K_{A} for a FcR of at least about 2×10¹⁰ M⁻¹, and even more preferably of at least about 1×10¹¹ M⁻¹. Also preferred is a mutein having a kₐ for a FcR of at least about 1×10⁵ liters/mole-second as well as a mutein having a k_{d} for a FcR of less than or equal to 3×10⁻⁵/second. More preferred is a mutein having a kₐ for a FcR of at least about 3×10⁵ liters/mole-second, and even more preferably of 1×10⁶ liters/mole-second. Also preferred are muteins having a k_{d} for a FcR of less than or equal to 1×10⁻⁵/second or even more preferably less than or equal to 3×10⁻⁴/second. A preferred FcR is FcεRIα. Methods to measure such binding constants is well known to those skilled in the art; see, for example, Cook et al., 1997, ibid., which reports the following values for the binding of human FcεRIα protein to human IgE: kₐ₁ of 3.5 (±0.9)×10⁵ M⁻¹s⁻¹; kₐ₂ of 8.6 (±3.5)×10⁴ M⁻¹s⁻¹; k_{d1} of 1.2 (±0.1)×10⁻² s⁻¹; k_{d2} of 3.2 (±0.8)×10⁻⁵ s⁻¹; K_{A1} of 2.0×10⁷ M⁻¹; K_{A2} of 2.9×10⁹ M⁻¹.

Another embodiment of the present invention is a mutein that exhibits altered substrate specificity compared to its unmodified counterpart. A mutein exhibiting altered substrate specificity is a mutein that binds with increased affinity to a FcR for an antibody class or antibody species of a different type than that normally bound by its unmodified counterpart. In one embodiment, a mutein of a human Fc-Cε3/Cε4 region with altered substrate specificity is a Fc region that binds with increased affinity to a receptor that binds to an IgE antibody of another mammal, such as, but not limited to, a canine, feline, equine, murine, or rat IgE antibody. In another embodiment, a mutein of a human Fc-Cε3/Cε4 region with altered substrate specificity is a Fc region that binds with increased affinity to a Fc receptor for an antibody of another class, such as IgG, IgM, IgA, or IgD, with IgG being preferred. Such a mutein can also show altered species substrate specificity. Methods to determine whether a mutein exhibits altered substrate specificity are well known to those skilled in the art.

Yet another embodiment of the present invention is a mutein that exhibits increased solubility compared to its unmodified counterpart. Such a protein is less likely to form aggregates. Methods to determine whether a mutein exhibits increased solubility are well known to those skilled in the art.

As disclosed herein, the 3-D model substantially representing the coordinates in Table 1, Table 2 or Table 3 is advantageous in determining strategies for producing muteins having an improved function, e.g., for identifying targets to modify in order to obtain muteins having improved functions. Examples of targets include, but are not limited to, those regions of the Fc-Cε3/Cε4 region that directly or indirectly interact with a FcεRIα protein.

In accordance with the present invention, a mutein having an improved function can be produced by a method that includes replacing at least one amino acid based on information derived from analyzing a 3-D model of the present invention to produce the mutein having the improved function. Knowledge of the structure of the human Fc-Cε3/Cε4 region, for example, permits the rational design and construction of modified forms of the protein by permitting the prediction and production of substitutions, insertions, deletions, inversions and/or derivatizations that effect an improved function. That is, analysis of 3-D models of the present invention provide information as to which amino acid residues are important and, as such, which amino acids can be changed without harming the protein. In making amino acid replacements, it is preferred to use amino acid replacements that have similar numbers of atoms and that allow conservation of salt bridges, hydrophobic interactions and hydrogen bonds unless the goal is to purposefully change such interactions. The 3-D structure of the human Fc-Cε3/Cε4 region suggests that large deletions may not be desirable, particularly due to the relation between the various domains of the protein and the observation that most of the structure is well ordered in the crystal.

It is to be appreciated that although one amino acid replacement capable of improving the function of a protein can substantially improve that function, more than one amino acid replacement can result in cumulative changes depending on the number and location of the replacements. For example, although one amino acid replacement capable of substantially increasing the stability of a protein can increase the melting temperature of that modified protein by about 1° C, about 5 to about 6 replacements may increase the melting temperature of the resultant protein by about 10° C.

In accordance with the present invention, the 3-D model of the Fc region has been analyzed, using techniques known to those skilled in the art, to determine the accessibility of the amino acids represented within the model to solvent. Such information is provided in, for example, Table 4 or Table 5.

A number of methods can be used to produce muteins of the present invention. One method includes the steps of: (a) analyzing a 3-D model substantially representing the coordinates specified in Table 1, Table 2 or Table 3 to identify at least one amino acid of the modeled protein which if replaced by a specified amino acid would effect an improved function; and (b) replacing the identified amino acid(s) to produce a mutein having that improved function. In one embodiment, a method to produce a mutein includes the steps of (a) comparing a key region of a model of a human Fc-Cε3/Cε4 region with the amino acid sequence of a Fc region having an improved function compared to the unmodified Fc-Cε3/Cε4 region in order to identify at least one amino acid segment of the Fc region with the improved function that if incorporated into the Fc-Cε3/Cε4 region represented by the model would give the Fc-Cε3/Cε4 region the improved function; and (b) incorporating the segment into the Fc-Cε3/Cε4 region, thereby providing a mutein with the improved function. In another embodiment, a method to produce a protein includes the steps of: (a) using a model representing a human Fc-Cε3/Cε4 region to identify a 3-D arrangement of residues that can be randomized by mutagenesis to allow the construction of a library of molecules from which a improved function can be selected; and (b) identifying at least one member of the mutagenized library having the improved function. In one example, a mutein is produced by a method that includes the steps of: (a) effecting random mutagenesis of nucleic acid molecules encoding a target of a Fc-Cε3/Cε4 region as identified by analyzing a model of that protein, such as an FcR binding domain; (b) cloning such mutagenized nucleic acid molecules into a phage display library, wherein said phage display library expresses the target; and (c) identifying at least one member of the library that expresses a target with an improved function, such as an FcR binding domain exhibiting increased affinity for an FcR. As stated above, the model allows the use of this technique in a straightforward manner that could not be accomplished in the absence of the model. It is to be also noted that these methods can also be used with other models of the present invention to produce muteins of the present invention.

The present invention includes a number of methods, based on analysis of a 3-D model of the present invention, to replace (i.e., add, delete, substitute, invert, derivatize) at least one amino acid residue in the protein represented by the model in order to produce a mutein of the present invention. Such methods include, but are not limited to: (a) replacing at least one amino acid in at least one non-constrained loop; (b) joining an amino-terminal amino acid residue to a carboxyl-terminal amino acid residue; (c) replacing at least one amino acid site with an amino acid suitable for derivatization; (d) replacing at least one pair of amino acids of the protein with a cysteine pair to enable the formation of a disulfide bond that stabilizes the protein; (e) replacing at least one amino acid in the FcεRIα binding domain in order to increase the affinity between an IgE Fc region and the corresponding FcR; (f) replacing at least one amino acid of the protein with an amino acid such that the replacement decreases the entropy of unfolding of the protein; (g) replacing at least one asparagine or glutamine of the protein with an amino acid that is less susceptible to deamidation than is the amino acid to be replaced; (h) replacing at least one methionine, histidine or tryptophan with an amino acid that is less susceptible to an oxidation or reduction reaction than is the amino acid to be replaced; (i) replacing at least one arginine of the protein with an amino acid that is less susceptible to dicarbonyl compound modification than is the amino acid to be replaced; (j) replacing at least one amino acid of the protein susceptible to reaction with a reducing sugar sufficient to reduce protein function with an amino acid less susceptible to that reaction; (k) replacing at least one amino acid of the protein with an amino acid capable of increasing the stability of the inner core of the protein; (l) replacing at least one amino acid of the protein with at least one N-linked glycosylation site; (m) replacing at least one N-linked glycosylation site of the protein with at least one amino acid that does not comprise an N-linked glycosylation site; and (n) replacing at least one amino acid of the protein with an amino acid that reduces aggregation of the protein. Muteins of the present invention can be produced using methods and rationales similar to those disclosed in PCT WO 00/26246, ibid.; such methods, which are incorporated herein by reference in their entirety, can be applied to Fc-Cε3/Cε4 muteins of the present invention.

Amino acid replacements can be carried out using recombinant DNA techniques known to those skilled in the art, including site-directed mutagenesis (e.g., oligonucleotide mutagenesis, random mutagenesis, polymerase chain reaction (PCR)-aided mutagenesis, gapped-circle site-directed mutagenesis) or chemical synthetic methods of a nucleic acid molecule encoding the desired protein, such as, but not limited to a human FcεRIα protein, followed by expression of the mutated gene in a suitable expression system, preferably an insect, mammalian, bacterial, yeast, insect, or mammalian expression system. See, for example, Sambrook et al., ibid.

It is to be appreciated that muteins of the present invention can include amino acids which are not modified because they would negatively impact the function of the protein. Such amino acids can be identified using a 3-D model of the present invention.

It should also be appreciated that it is within the scope of the present invention to expand the use of models of the present invention to produce models of and make modifications to any suitable FcRs or other Ig domain-containing proteins to produce muteins having a desired function.

Antibody muteins have a variety of uses, including but not limited to, diagnostic and therapeutic uses. For example, muteins could be used to image cells that express an antibody receptor protein, such as NMR-specific labeling for in vivo imaging to detect, for example, mast cell cancers, asthma, and other pathologies, or to treat cancers that express an antibody receptor protein using, for example, radioimmune therapy of derivatized IgE. Muteins could also be used for monitoring FcR expression in atopic individuals (e.g. with a tag for one-step FACS analysis) or for monitoring IgE in atopic individuals. Muteins could also be used as inhibitors or as toxin-IgE-Fc fusion proteins to target FcR-expressing cells to kill them (e.g. in mast cell tumors or severe allergy). Also muteins that affect the low affinity affinity IgE-receptor (FceRII) binding but not FceRI binding could be designed or selected.

The present invention also includes nucleic acid molecules that encode muteins of the present invention as well as recombinant molecules and recombinant cells that include such nucleic acid molecules. Methods to produce such proteins are also disclosed herein.

The present invention also includes the following novel structures as identified by a 3-D model of the present invention. Preferred structures exhibiting direct interaction between IgE and FcεRIα include a FcεRIα binding domain, an interdomain groove between the two Cε3/Cε4 domains of said antibody Fc region, a hinge between Cε3 and Cε4 domains of said antibody Fc region, and a region of a Cε3 or Cε4 domain, the relative position of which changes by greater than 1 angstrom between closed and receptor-bound Fc-Cε3/Cε4 conformations. Preferred compositions include a linker between Cε2 and Cε3, a BC loop of Cε3, a DE loop of Cε3, and a FG loop of Cε3, a loop or strand defining the interdomain groove, a AB helix of Cε3 and, a region lying above said AB helix of Cε3. The present invention also includes nucleic acid molecules to encode such compositions.

The present invention also includes an isolated Fc-Cε3/Cε4 protein selected from the group consisting of: (a) a protein consisting of SEQ ID NO:2; and (b) an isolated protein that is structurally homologous to a protein of (a), wherein said protein of (b) binds to a FcεRIα protein. Also included in the present invention is such a protein produced in insect cells. In one embodiment the Fc-Cε3/Cε4 protein a human Fc-Cε3/Cε4 protein, a canine Fc-Cε3/Cε4 protein, a feline Fc-Cε3/Cε4 protein, an equine Fc-Cε3/Cε4 protein, a murine Fc-Cε3/Cε4 protein, or a rat Fc-Cε3/Cε4 protein. The present invention also includes nucleic acid molecules that encode such proteins, as well as recombinant molecules, recombinant cells and recombinant viruses that include such nucleic acid molecules. Also included are methods to produce such proteins using such nucleic acid molecules, recombinant molecules, recombinant viruses and recombinant cells.

The present invention also includes isolated nucleic acid molecules encoding proteins of the present invention, including, but not limited to, unmodified proteins, novel structures within such proteins, and muteins. As used herein, an isolated nucleic acid molecule encoding a protein is a nucleic acid molecule that has been removed from its natural milieu. As such, “isolated” does not reflect the extent to which the nucleic acid molecule has been purified. An isolated nucleic acid molecule can be DNA, RNA, or derivatives of either DNA or RNA.

A nucleic acid molecule encoding a mutein of the present invention can be produced by mutation of parental protein genes (e.g., unmodified or previously modified protein-encoding genes, or portions thereof) using recombinant DNA techniques heretofore disclosed or by chemical synthesis. Resultant mutein nucleic acid molecules can be amplified using recombinant DNA techniques known to those skilled in the art, such as PCR amplification or cloning (see, for example, Sambrook et al., ibid.), or by chemical synthesis. A mutein can also be produced by chemical modification of a protein expressed by a nucleic acid molecule encoding an unmodified protein or mutein-encoding gene.

Proteins of the present invention can be produced in a variety of ways, including production and recovery of recombinant proteins and chemical synthesis. In one embodiment, a protein of the present invention is produced by culturing a cell capable of expressing the protein under conditions effective to produce the protein, and recovering the protein. A preferred cell to culture is a recombinant cell that is capable of expressing the protein, the recombinant cell being produced by transforming a host cell with one or more nucleic acid molecules of the present invention. Transformation of a nucleic acid molecule into a host cell can be accomplished by any method by which a nucleic acid molecule can be inserted into a cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of a host cell in such a manner that their ability to be expressed is retained.

Suitable host cells to transform include any cell that can be transformed. Host cells can be either untransformed cells or cells that are already transformed with at least one nucleic acid molecule. Host cells of the present invention can be endogenously (i.e., naturally) capable of producing a protein of the present invention, but such cells are not preferred. Host cells of the present invention can be any cell that when transformed with a nucleic acid molecule of the present invention are capable of producing a protein of the present invention, including bacterial, yeast, other fungal, insect, animal, and plant cells. Preferred host cells include bacterial, yeast, insect and mammalian cells, and more preferred host cells include Escherichia, Bacillus, Saccharomyces, Pichia, Trichoplusia, Spodoptera and mammalian cells. Particularly preferred host cells are Trichoplusia ni cells and Spodoptera frugiperda cells with T. ni cells being particularly preferred.

A recombinant cell is preferably produced by transforming a host cell with a recombinant molecule comprising a nucleic acid molecule of the present invention operatively linked to an expression vector containing one or more transcription control sequences. The phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell, of replicating within the host cell, and of effecting expression of a specified nucleic acid molecule. Expression vectors can be either prokaryotic or eukaryotic, and are typically viruses or plasmids. Expression vectors of the present invention include any vectors that function (i.e., direct gene expression) in recombinant cells of the present invention, including in bacterial, yeast, other fungal, insect, animal, and plant cells. Preferred expression vectors of the present invention can direct gene expression in bacterial, yeast, insect and mammalian cells.

Nucleic acid molecules of the present invention can be operatively linked to expression vectors containing regulatory control sequences such as promoters, operators, repressors, enhancers, termination sequences, origins of replication, and other regulatory control sequences that are compatible with the host cell and that control the expression of the nucleic acid molecules. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, insect and mammalian cells.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, deletion of sequences that destabilize transcripts, and use of control signals that temporally separate recombinant cell growth from recombinant protein production during fermentation. The activity of an expressed recombinant protein of the present invention may be improved by fragmenting, modifying, or derivatizing nucleic acid molecules encoding such a protein.

In accordance with the present invention, recombinant cells can be used to produce proteins by culturing such cells under conditions effective to produce such a protein, and recovering the protein. Effective conditions to produce a protein include, but are not limited to, appropriate media, bioreactor, temperature, pH and oxygen conditions that permit protein production. An appropriate medium refers to any medium in which a cell of the present invention, when cultured, is capable of producing the protein. An effective medium is typically an aqueous medium comprising assimilable carbohydrate, nitrogen and phosphate sources, as well as appropriate salts, minerals, metals and other nutrients, such as vitamins. The medium may comprise complex nutrients or may be a defined minimal medium. Cells of the present invention can be cultured in conventional fermentation bioreactors, which include, but are not limited to, batch, fed-batch, cell recycle, and continuous fermentors. Culturing can also be conducted in shake flasks, test tubes, microtiter dishes, and petri plates. Culturing is carried out at a temperature, pH and oxygen content appropriate for the recombinant cell. Such culturing conditions are well within the expertise of one of ordinary skill in the art.

Depending on the vector and host system used for production, resultant proteins may either remain within the recombinant cell; be secreted into the fermentation medium; be secreted into a space between two cellular membranes, such as the periplasmic space in E. coli; or be retained on the outer surface of a cell or viral membrane. The phrase “recovering the protein” refers simply to collecting the whole fermentation medium containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization.

The present invention also includes isolated (i.e., removed from their natural milieu) antibodies that selectively bind to a Fc region of the present invention. As used herein, the term “selectively binds to” refers to the ability of antibodies of the present invention to preferentially bind to an Fc region of the present invention. Binding can be measured using a variety of methods standard in the art including enzyme immunoassays (e.g., ELISA), immunoblot assays, etc.; see, for example, Sambrook et al., ibid. Isolated antibodies of the present invention can include antibodies in a bodily fluid (such as, but not limited to, serum), or antibodies that have been purified to varying degrees. Antibodies of the present invention can be polyclonal or monoclonal. Functional equivalents of such antibodies, such as antibody fragments and genetically-engineered antibodies (including single chain antibodies or chimeric antibodies that can bind to more than one epitope) are also included in the present invention. Antibodies can be produced using methods known to those skilled in the art. A preferred method to produce antibodies of the present invention includes (a) administering to an animal an effective amount of a protein of the present invention to produce the antibodies and (b) recovering the antibodies. In another method, antibodies of the present invention are produced recombinantly using techniques as heretofore disclosed to produce proteins of the present invention. Antibodies raised against defined proteins can be advantageous because such antibodies are not substantially contaminated with antibodies against other substances that might otherwise cause interference in a diagnostic assay or side effects if used in a therapeutic composition.

Antibodies of the present invention have a variety of potential uses that are within the scope of the present invention. Examples of such uses are disclosed in WO 98/27208, ibid., see, for example, page 24; such uses are incorporated by reference herein in their entireties.

A Fc region of the present invention can include chimeric molecules comprising at least a portion of a Fc region that binds to an antibody and a second molecule that enables the chimeric molecule to be bound to a substrate in such a manner that the antibody receptor portion binds to the antibody in at least as effective a manner as a Fc region that is not bound to a substrate. An example of a suitable second molecule includes a portion of an immunoglobulin molecule or another ligand that has a suitable binding partner that can be immobilized on a substrate, e.g., biotin and avidin, or a metal-binding protein and a metal (e.g., His), or a sugar-binding protein and a sugar (e.g., maltose).

The present invention includes uses of Fc regions, antibodies thereto, and inhibitory compounds of the present invention for the diagnosis and treatment of allergy and the regulation of other immune responses in an animal.

One embodiment is a therapeutic composition comprising at least one of the following therapeutic compounds: an inhibitory compound of the present invention, a mutein of the present invention, or an antibody of the present invention. Also included is a method to protect an animal from allergy or other abnormal immune responses. Such a method includes the step of administering a therapeutic composition of the present invention to the animal. As used herein, the ability of a therapeutic composition of the present invention to protect an animal from allergy or other abnormal immune responses refers to the ability of that composition to, for example, treat, ameliorate or prevent allergy or other abnormal immune responses. General characteristics of therapeutic compositions and methods to produce and use such therapeutic compositions are disclosed, for example, in WO 98/27208, ibid., see, for example, page 39-47; such compositions and methods are incorporated by reference herein in their entireties. It is to be noted that although the compositions and methods disclosed in WO 98/27208, ibid., relate to feline FcεRIα proteins, they are also applicable to therapeutic compositions of the present invention. Therapeutic compositions of the present invention are advantageous because they can be derived from analysis of 3-D models of the present invention and have improved functions, such as efficacy and safety.

Another embodiment is a diagnostic reagent comprising a mutein of the present invention. As used herein, a diagnostic reagent is a composition that includes a mutein that is used to detect allergy or other abnormal immune responses in an animal. Also included in the present invention are methods, including in vivo methods and in vitro methods, to (a) detect allergy or other abnormal immune response, or susceptibility thereto, in an animal, comprising use of a diagnostic reagent comprising a mutein of the present invention and (b) to enhance the performance of an IgE or FcR binding assay, said method comprising incorporating into the assay a mutein of the present invention. General characteristics of diagnostic reagents and methods to produce and use such diagnostic reagents are disclosed, for example, in WO 98/27208, ibid., see, for example, page 2-39; such reagents and methods are incorporated by reference herein in their entireties. It is to be noted that although the reagents and methods disclosed in WO 98/27208, ibid., relate to feline FcεRIα proteins, they are also applicable to diagnostic reagents, kits and detection methods of the present invention. Muteins of the present invention are advantageous in such applications because of their enhanced affinity for antibodies, altered specificity, enhanced solubility and/or enhanced stability, enabling for example use in otherwise adverse conditions and longer shelf-life.

The following examples are provided for the purposes of illustration and are not intended to limit the scope of the invention.

### EXAMPLES

### Example 1

This Example describes the production and analysis of a crystal and model of the present invention. It is to be noted that numbering of Fc-Ce3/Ce4 residues follows the convention of Dorrington et al, ibid.

The binding of soluble IgE to its high affinity receptor, FcεRI, is a requisite step in the cascade of events associated with the allergic response and anti-parasitic immunity¹⁻³. Crosslinking of receptor-bound IgE by antigens triggers intracellular signaling events leading to effector cell activation⁴. This example describes the solution of a 2.3 Å crystal structure of the human IgE-Fc domains, Cε3 and Cε4, which bind to FcεRI, the coordinates of which are disclosed in Table 1. The IgE-Fc crystal structure reveals a large (˜15•) tertiary rearrangement of the Cε3 domains when compared to IgG-Fc structures and the IgE-Fc bound to FcεRI. The free IgE-Fc adopts a more compact arrangement that places the Cε3 domains into close proximity in a “closed” configuration, obstructing receptor-binding loops. This IgE-Fc conformational change is mediated by three flexible segments that lie within the Cε3 domain and not by the interdomain connecting loop. The “closed” structure of the IgE-Fc highlights the potential for novel conformational variation in the effector domains of different antibody classes and suggests new strategies for the design of therapeutic compounds for the treatment of allergy and asthma.

The interaction of antibody Fc domains with cellular antibody receptors couples the diversity of the antibody repertoire to many effector cells of the immune system. Fc receptors specific for antibody subclasses, including IgE, IgG and IgA antibodies, are found on overlapping but distinct subsets of cells of the hematopoetic system and can thereby trigger distinct mechanisms of the immune response^{1,4,5}. IgE-mediated immune reactions are implicated in parasitic infections, allergy and asthma.

IgE antibodies consist of two Fabs and an Fc that is formed by a dimer of three constant domains (Cε2, Cε3, and Cε4). Compared to IgG molecules, IgE has an additional constant domain (Cε2) that replaces the IgG linker region, while the IgE Cε3 and Cε4 domains are homologous to the IgG Cγ2 and Cγ3 domains. Intact IgE and Fc fragments bind with high affinity (K_{D}˜10⁻⁹-10⁻¹⁰ M) to the alpha chain of FcεRI and mutagenesis studies⁶⁻¹¹ have demonstrated that Cε3 domain residues are involved in binding to the receptor, consistent with the crystallographic studies of the IgE-Fc:FcεRIα complex. The IgE-Cε2 domains are not thought to be important for receptor binding, since constructs of the IgE-Cε3/Cε4 domains retain high affinity binding to the receptor.

In order to obtain protein for crystallographic studies, the C-terminal IgE-Fc domains, Cε3/Cε4, were expressed in insect cells and purified as described in the methods section. Crystals were obtained that diffract weakly using laboratory X-ray sources but diffract to 2.3 Å using high energy synchrotron X-ray sources. The IgE-Fc Cε3/Cε4 structure was solved by an automated molecular replacement search strategy using ˜12,000 distinct conformational variants of core models for the two immunoglobulin domains, in which the domain orientations were varied by rotation about three axes centered near the connecting loop between the two domains. A course (3• angular) search yielded a single promising solution that was further refined by finer domain rotations. The structure was subjected to rounds of refinement and building into simulated annealing composite omit electron density maps, giving the final statistics shown in Table 6. The Rfree and Rfactor are, respectively, with good geometry to 2.3 Å resolution.

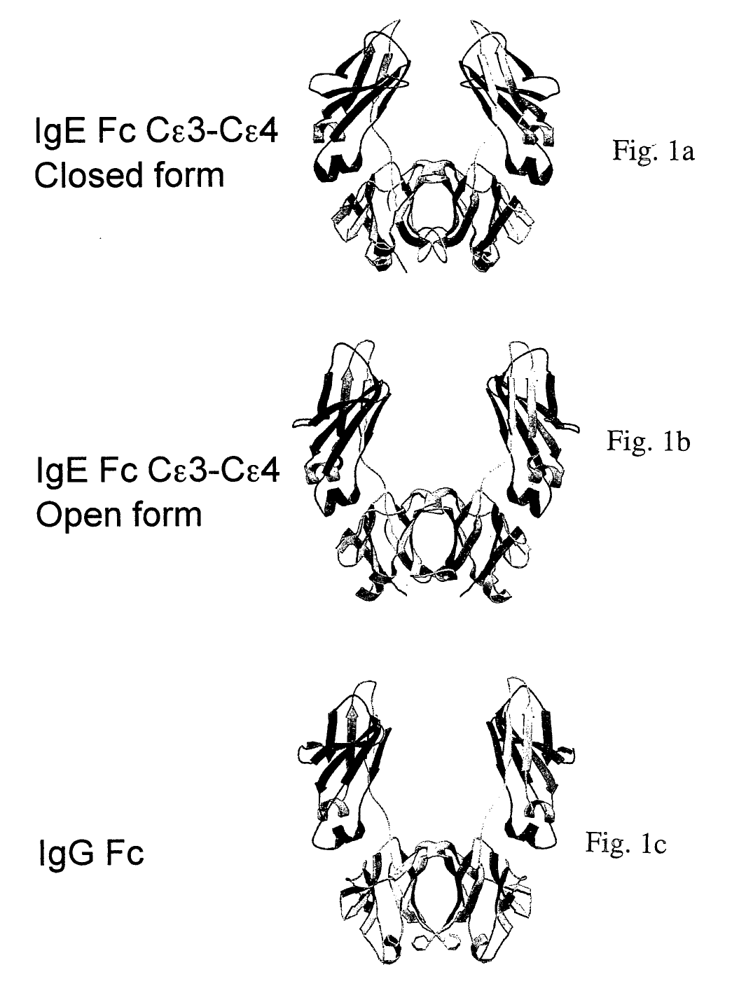

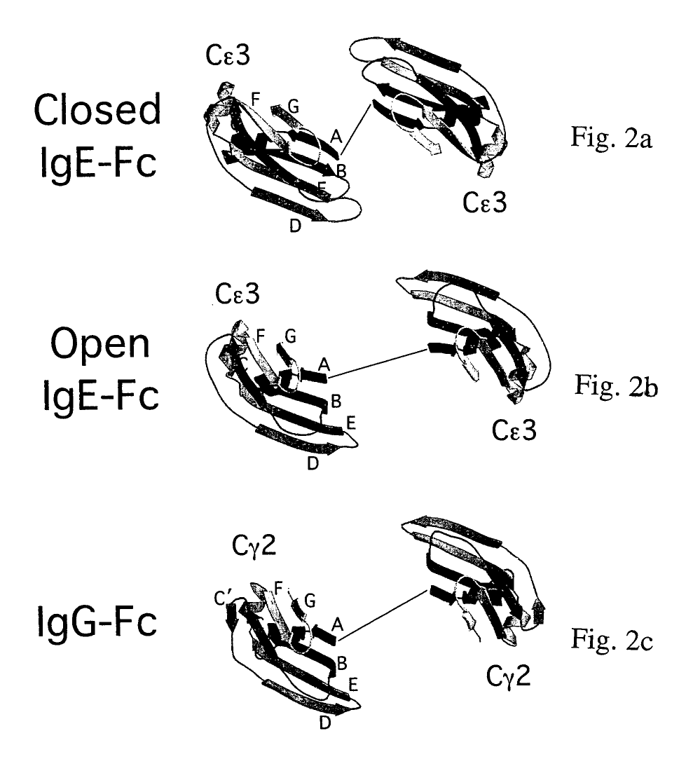

The overall structure for the IgE Cε3/Cε4 domains is shown in FIGS. 1a and 2a. The two immunoglobulin domains belong to the C1 set of antibody constant domains and are individually similar to the structures for the receptor-bound IgE-Fc and IgG-Fc domains shown in FIGS. 1b, 2b and 1c, 2c, respectively. No density for residues N-terminal to V336 (the Cε2/Cε3 linker) are observed in the IgE-Fc structure, despite the fact that an interchain disulfide occurs in this region and can be shown to form biochemically. The Cε2/Cε3 linker becomes ordered and visible in electron-density maps upon binding FcεRI (see, for example, Ser. No. 60/189,853). Similar to the IgG-Fc, the Cε3 domains of the heavy chain dimer do not form any inter-chain contacts, while the Cε4 domains form an extensive dimer interface, burying ˜1860 Å² of surface area. Conserved carbohydrate found at N394 in IgE, fills the cavity between the Cε3 and Cε4 domains and makes limited contacts across the dimer interface. In contrast to IgG-Fc structures; IgE carbohydrate can be removed and binding to FcεRI is retained⁸.

The IgE-Fc crystal structure reveals a novel and compact closed conformation for the Fc domains. The relative dispositions of the two Cε3 domains with respect to each other and to the Cε4 domains is substantially different from IgG-Fc or the receptor-bound IgE-Fc structures (FIG. 1). The Cε3/Cε4 angle is more acute than that found between IgG-Fc Cγ2/Cγ3 domains or for the receptor-bound IgE-Fc (FIG. 1). The free IgE-Fc is more compact, as shown by its shorter overall height (FIG. 1a, ˜7 Å), with overall dimensions of 58×63×40 Å as compared to 65×64×36 Å for IgG-Fc (FIG. 1c) and to the dimensions of the receptor-bound IgE-Fc (FIG. 1b) (see, for example, Ser. No. 60/189,853). The Cε3 domains in the IgE-Fc structure also approach each other more closely, as shown by the distances between loop residues indicated in FIG. 2a. The distances between the first residue in strand A of the Cε3 or Cγ2 Ig domains can be readily compared, minimizing differences in distance due to loop flexibility. In IgG (FIG. 2c), this distance is ˜22 Å (varies some between IgG structures), in the receptor-bound IgE-Fc (FIG. 2b) this distance is ˜23 Å, while in the closed IgE (FIG. 2a) this distance is ˜13 Å. Overall, the receptor-bound IgE and IgG-Fc structures more closely resemble each other than the unbound IgE-Fc structure.

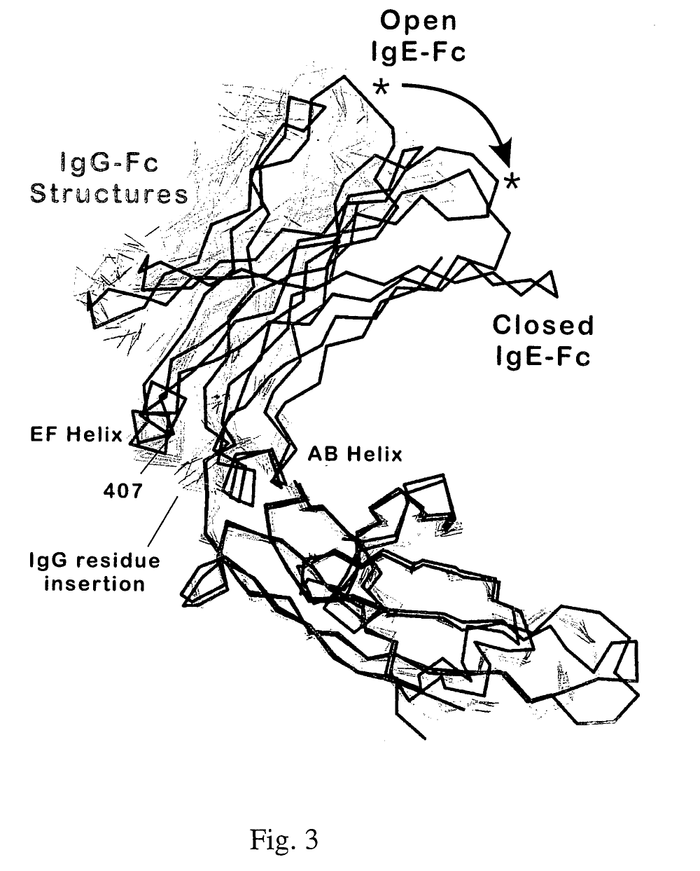

Although conformational differences in IgG-Fc structures have been noted^{12,13}, these are not as large as observed for the receptor-bound and closed IgE-Fc structures. FIG. 3 shows a superposition of nine different IgG structures along with the closed IgE-Fc structure observed here. The IgE-Fc lies significantly beyond the observed range of motion in IgG-Fc structures. A superposition of the receptor-bound and closed forms of the IgE-Fc is shown in FIG. 3, demonstrating the large conformational change observed. The AB helix of Cε3 and the interdomain linker residues remain relatively fixed with respect to the Cε4 domain, as shown by a superposition of the open and closed IgE conformations (FIG. 3). The largest IgG-Fc conformational differences are found in comparisons of human and mouse IgG-Fc structures that are 65% identical in sequence¹², potentially accounting for some of the conformational differences. In contrast, the IgE-Fc structures compared here demonstrate large conformational flexibility for a molecule of identical sequence.

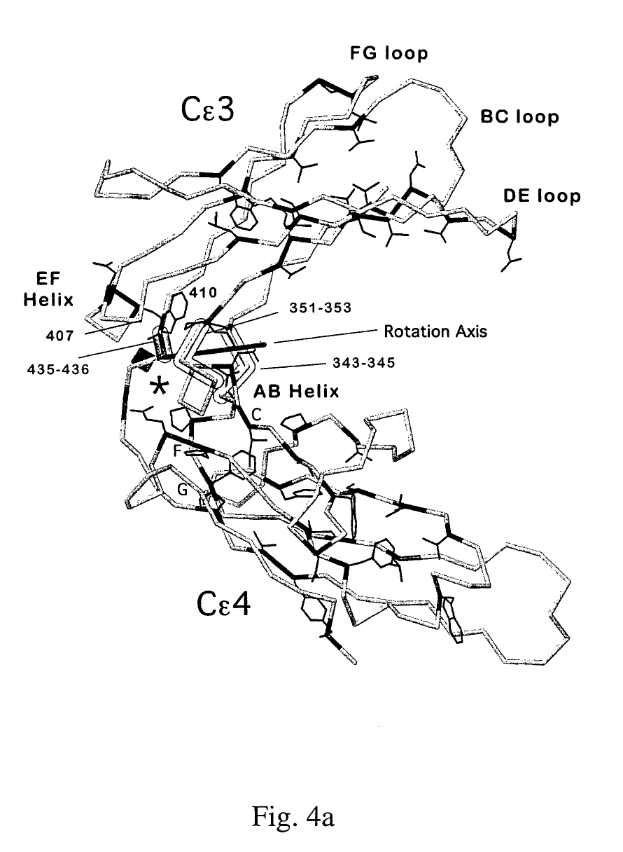

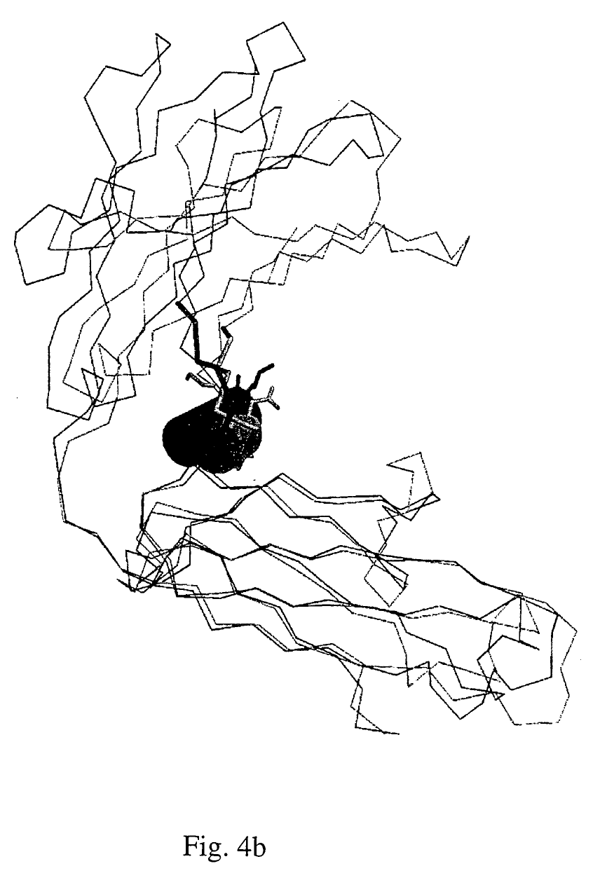

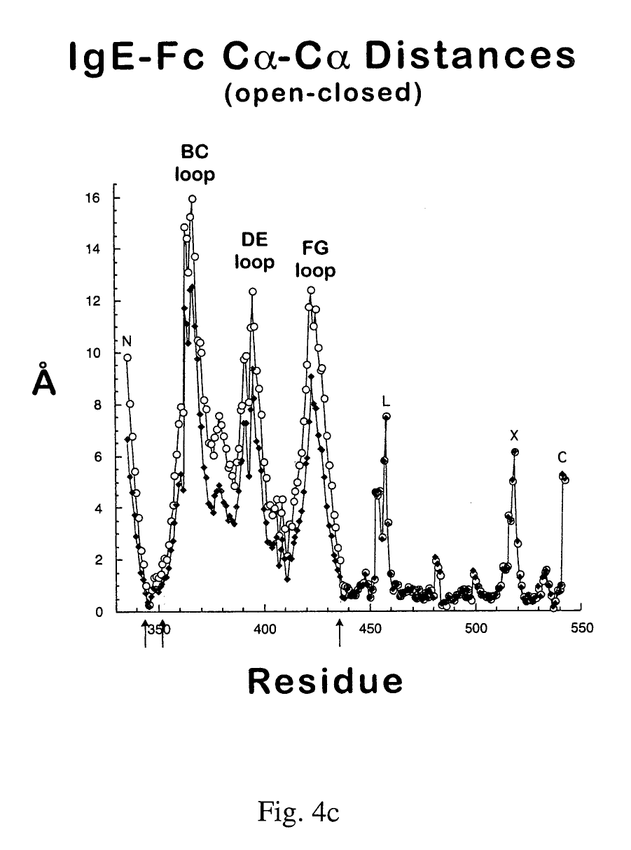

An analysis of the IgE-Fc conformational difference is shown in FIG. 4a, in which comparison of the closed and open forms was carried out with the program DynDom¹⁴. Dyndom groups residues that move as semi-rigid domains for proteins in different conformational states and identifies an interdomain screw axis and hinge residues for the IgE domain motion. The axis of the IgE bending motion is indicated by the arrow in FIG. 4a, with an approximately 15• rotation and 0.6 Å translation relating the open and closed forms. The bend between the two domains does not occur in the interdomain region (residues 436-440) but rather occurs within the Cε3 domain itself. The residues that constitute the hinge lie above the Cε3-AB helix and include amino acids 342-344 (after strand A), 354-356 (before strand B), and 434-436 (after strand G) and are highlighted with light purple in FIG. 4a.

The structural basis for the difference in apparent flexibility in the IgG- and IgE-Fc domains is not simply based on sequence differences in the linker amino acids between the two immunoglobulin domains, given the potential for hinge motion in the three peptide segments identified by DynDom. The interdomain linker region and the AB helix remain relatively fixed in the comparison of closed and open IgE-Fc structures (FIGS. 1c and 4a). At the C-terminal end of the AB helix, small structural changes account for a single residue insertion in IgG-Cγ2 sequences compared to IgE-Cε3 and these are not part of the hinge regions in IgE (FIG. 4b). In both IgE and IgG, the A and B β-strands of Cε3 or Cγ2 separate and do not hydrogen bond on either side of the AB helix, and it is in this region that the IgE bending appears to concentrate (FIGS. 4a and 4b). Comparison of the IgG-Fc and IgE-Fc structures shows a concerted shift in the position of the IgE-Fc AB helix outwards from the Cε3/Cε4 interface, which may account for the greater rotational freedom in the IgE-Fc structures (FIG. 4b). In the closed IgE-Fc structure, the hinge residues immediately after the AB helix (amino acids 354-356) sterically clash with the superimposed IgG-Fc AB helix (FIG. 4b). Thus the closed conformation observed here for the IgE-Fc may be inaccessible to the IgG-Fc, because of the position of the IgG-Fc AB helix. Additional residues may also contribute to the conformational differences in IgG and IgE, such as the change of P257/P258 in IgG for R342/P343 in IgE in the A strand and the presence of P354 at the beginning of the IgE-AB helix. Differences in the phi/psi angles accessible to the double proline sequence in IgG could additionally stabilize the open configuration of IgG-Fcs. In contrast to human IgE-Fc, the mouse and rat sequences contain this double proline motif. It will be interesting to test if mutation of R342 for proline stabilizes the open conformation as measured by solution techniques and if this mutation has an effect on the binding affinity for IgE receptors.

The large conformational change observed in the closed IgE-Fc structure reorients loops at the top of the Cε3 domains that interact with FcεRI. FIG. 4c quantifies these conformational differences by comparing the distance between pairs of Cα carbon atoms in the free IgE-Fc and the receptor-bound IgE-Fc. Loops that are involved in binding FcεRI are highlighted above the graph and these regions are observed to move by 10-14 Å between the bound and free forms of the IgE-Fc. Since the rotation axis lies near the base of the Cε3 domain (FIG. 4a), a gradient of increasing Cα displacements can be seen in this plot that corresponds to amino acids in the Cε3 beta strands. The peaks in the plot correspond to loops at the top of the Cε3 domain that show the largest displacements (FIG. 4c). The AB-helix in the Cε3 domain appears to be linked structurally to the Cε4 domains by this analysis as well as the domain-movements shown in FIG. 3.

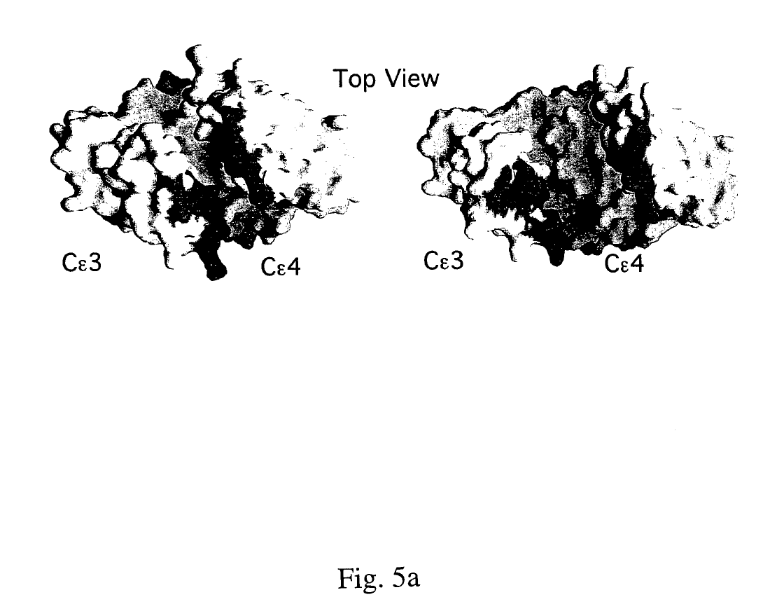

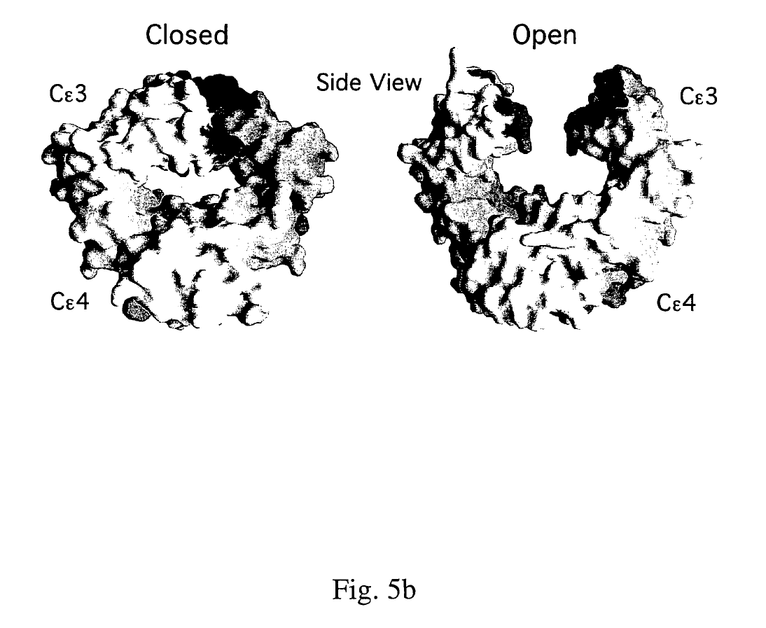

The movement of the IgE-Fc receptor-binding loops by 10-14 Å suggests that these loops would be poorly positioned in the “closed” form to interact with the receptor-binding surface. FIGS. 5a and 5b show surface representations of the FcR-binding loops of the Cε3 domain for the bound and free forms. The rotation of the Cε3 domains about this internal hinge reorients the receptor-binding surfaces. In the open, receptor-binding form, the Cε3 loops are exposed and together with the N-terminal linker residues to the Cε2 domains, form a crown-like structure that interacts with the broad and convex surface of FcεRI. In contrast, in the closed Fc conformation, the receptor-binding loops are reoriented to point towards each other across the IgE-Fc diad axis, forming a narrower inter-domain gap that cannot accommodate the binding of the receptor. The relatively large space separating Cγ2 domains in IgG and in the Cε3 domains of “open” form of IgE, becomes more of a cleft in the closed IgE-Fc (FIG. 5a).

The IgE-Fc conformation in solution is likely to be dynamic and the full range of Cε3 conformational mobility remains to be established. However, biophysical studies of IgE conformation in solution have previously suggested more compact models for the IgE conformation as compared to IgG antibodies. Neutron scattering studies of a three domain construct of the IgE-Fc (Cε2/Cε3/Cε4) are consistent with a more compact structure than that of IgG-Fc in solution¹⁵. In addition, N-terminal to C-terminal distances for intact IgE have also been measured by fluorescence energy transfer (˜71 Å) and suggest a bent conformation for the antibody in solution, with less hinge-mediated flexibility as compared to IgG^{16,17}. These studies may also be consistent with the more compact IgE-Fc conformation observed in the crystal structure reported here.

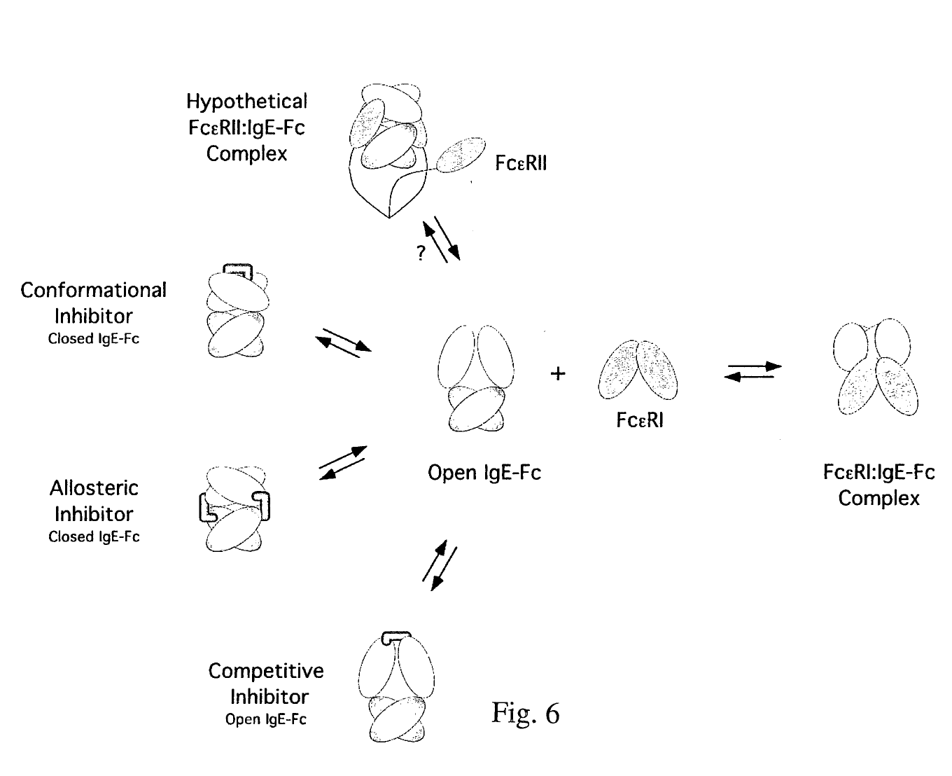

The observed conformational flexibility in the IgE-Fc may be important for unique aspects of IgE biological function. IgE-Fc flexibility may allow for induced-fit interactions with the FcεRI, contributing to the high affinity binding or may also be important in interactions with the low affinity IgE receptor, FcεRII (FIG. 6). FcεRII is a trimeric C-type lectin that is thought to interact with the IgE-Fc through two of the three lectin domains¹⁸. IgG antibodies do not interact with a corresponding lectin-like receptor, consistent with a potential biological role for IgE-Fc conformational variation in this unique antibody-receptor interaction. Finally, the observation of the closed IgE-Fc conformation provides a template for the design of new inhibitors of the IgE-FcεRI interaction (FIG. 6). Stabilization of the closed IgE-Fc conformation by the binding of small molecules, either at directly competitive or indirect allosteric sites, could block receptor association (FIG. 6), leading to a new class of therapeutic inhibitors for the treatment of IgE-mediated allergic diseases. The recent observation that in vitro selected peptides bind to the hinge region of the IgG-Fc, suggests the design of peptides that bind selectively to the closed IgE-Fc hinge to block FcεRI binding¹⁹.

Methods

Expression and Crystallization of the Human IgE-Fc

The IgE-Fc Cε3/Cε4 was expressed in insect cells using the Pharmingen baculogold expression system. Briefly, DNA encoding the Cε3/Cε4 domains were subcloned into the pACgp67 expression vector and recombinant virus generated by established methods. IgE-Fc Cε3/Cε4 protein expression was monitored by a receptor-based ELISA assay and protein was purified by conventional techniques (ion exchange, gel filtration and hydroxyapatite chromatography) from 10-20 liters of supernatants of infected T. ni cells. The Fc construct codes for an N-terminal sequence (ADPCDSN) that includes four amino acids (ADPC) upstream of the native sequence beginning with residue D330. The cysteine is displaced by one residue from the natural IgE-Fc cys328, but forms intramolecular disulfide binds in >95% of the isolated IgE-Fc.

Purified IgE-Fc was concentrated to 10 mg/ml in 10 mM Tris, pH 8.0, using an e280 nm of 1.32 cm−1 (mg/mL)−1. Crystals were obtained using the hanging drop method by mixing 0.5 ml protein +0.5 ml of precipitant (25 mM Sodium Acetate, pH 4.6, 33% (w/v) polyethylene glycol 4000). Crystals grew at room temperature in 1-3 days and were sensitive to small changes in salt or PEG concentration as well as temperature. Crystals were harvested into 25 mM Sodium Acetate, pH 4.6, 37% PEG 4000 and transferred to a cryoprotectant solution (harvest buffer plus 15% (v/v) ethylene glycol) for ˜30 seconds prior to flash-freezing in liquid nitrogen. Crystals belong to the space group P42₁2 with cell dimensions of a=b=105.6 Å, c=47.1 Å and α=β=γ=90• and contain one Cε3/Cε4 chain per asymmetric unit of the crystal.

Crystal Structure Determination and Refinement

Data from the IgE-Fc crystals was initially variably anisotropic, but improved substantially when crystals were treated with heavy atoms used for derivative screening, including platinum, mercury and other metals. Based on these onservations, crystals were treated with 1 mM copper (II) chloride prior to freezing and data collection. Although initial diffraction from native crystals was limited to ˜3.0 Å resolution and often exhibited split lattices, copper-treated crystals diffracted to at least 2.3 Å resolution, with little anisotropy lattice problems. This improvement may be due to the oxidation of residual free cysteines (<5%) in the IgE-Fc N-terminal residues prior to freezing. Data were collected from these crsytals at SSRL beamline 7-1 using a Mar300 imaging plate system and at the Advanced Photon Source DND-CAT 5Idbeamline, using a MarCCD detector. Native and derivative data were processed and integrated using the HKL suite of programs.

Initial attempts at solving the crystal structure by Molecular Replacement (MR) methods failed, using a variety of models of IgE based on IgG-Fc structures, including individual Ig domains and superpositions of IgG Fc structures among others. MR searches were carried out with Amore, CNS/XPLOR and EPMR without success. Heavy atom searches were carried out with a wide range of compounds (27), concentrations (0.1-20 mM) and pH ranges (4.6-8.5) but did not yield a well-behaved isomorphous derivative.

Since it was possible that the MR searches failed because of an altered conformation for the IgE-Fc as compared to the search model, a systematic exploration of the bend, twist and Cε3 rotation angles relative to Cε4 was undertaken. For this search, the Cγ2 and Cγ3 domains from the crystal structure of an intact mouse monoclonal antibody (PDB code 1IGT)²⁰ were used that were truncated to exclude non-homologous loops and side-chains, providing a model with 144 residues for 222 in the IgE-Fc construct. The two domains were translated to place the Cγ2/Cγ3 linker residues at the origin, with Cγ2 and Cγ3 oriented to allow a bending rotation to occur about the z-axis. Three rotations around X, Y and Z were applied to the Cγ2 domain, while leaving the Cγ3 fixed, using the program lsqkab from the CCP4 suite²¹. Approximately 12000 models were generated automatically and used in complete Molecular Replacement searches with the program Amore²¹, taking ˜10 days on 5 Silicon Graphics computers. The models covered an angular range of −30 to +40 degrees around the starting model with 3• increments in each rotation. A promising initial search solution was refined by restricting the search range and reducing the rotational stepsize to 0.5 degree increments. This fine search produced a model with a correlation coefficient of 38% and an Rfactor of 489% with data from 15-4 Å resolution. This model was subjected to rigid body refinement and a simulated annealing composite omit map was calculated to 3 Å resolution using the program CNS²². Interpretable density was observed in regions omitted from the search model and errors in the model could be easily identified. Model building and refinement were continued using the programs O²³ and CNS²². The current refinement statistics for all data from 0-2.3 Å resolution are collected in Table 6.

TABLE 6
Data Collection and Refinement
Data collection:

| crystal (pH of | | |
|---|---|---|
| harvest buffer) | | |
| (pH 7.5) | wtcu3 (pH 6.5) | wtcu1 |
| Source | APS, DND-CAT | SSRL, 7-1 |
| | (line = ?) | |
| Wavelength, | 0.906 Å, 13.67 keV | 1.08 Å, 11.48 keV |
| energy | | |
| Resolution | 2.30 Å | 2.60 Å |
| | (2.38-2.30 Å)† | (2.69-2.60 Å)† |
| Completeness | 99.8% (99.5%)† | 99.9% (99.0%)† |
| Total number | 106,855 | 83,604 |
| of reflections | | |
| Unique | 12,340 | 8,586 |
| reflections | | |
| Mosaicity | 0.48° | 0.65° |
| R_{merge} | 5.4% (25.7%)† | 6.6% (24.5%)† |
| Average | 8.66 (>7.1)† | 9.74 (>7.4)† |
| redundancy | | |
| <I/σI> | 33.9 (10.6)† | 13.9 (8.6)† |

†last shell

References

1. Kinet, Annu Rev Immunol 17, 931-972 (1999).

2. Metzger, Immunol Rev 125, 37-48 (1992).

3. Sutton et al., Nature 366, 421-428 (1993).

4. Daeron, Annu Rev Immunol 15, 203-234 (1997).

5. Ravetch et al., Annu Rev Immunol 16, 421-432 (1998).

6. Weetall et al., Immunol 145, 3849-3854 (1990).

7. Nissim et al., Embo J 10, 101-107 (1991).

8. Basu et al., J Biol Chem 268, 13118-13127 (1993).

9. Henry et al., Biochemistry 36, 15568-15578 (1997).

10. Presta et al., J Biol Chem 269, 26368-26373 (1994).

11. Sayers et al., Biochemistry 37, 16152-16164 (1998).

12. Harris et al., J Mol Biol 275, 861-872 (1998).

13. Harris. et al., Adv Immunol 72, 191-208 (1999).

14. Hayward et al., Proteins 30, 144-154 (1998).

15. Beavil et al., Biochemistry 34, 14449-14461 (1995).

16. Zheng et al., Biochemistry 30, 9125-9132 (1991).

17. Zheng et al., Biochemistry 31, 7446-7456 (1992).

18. Shi et al., Biochemistry 36, 2112-2122 (1997).

19. DeLano et al., Science 287, 1279-1283 (2000).

20. Harris et al., Nature 360, 369-372 (1992).

21. Collaborative Computational Project, Acta Cryst. D50, 760-763 (1994).

22. Brunger et al., Acta Crystallogr D Biol Crystallogr 54, 905-921 (1998).

23. Jones et al., Acta Crystallogr A 47, 110-119 (1991).

### Example 2

This Example describes the further refinement of the model described in Example 1. It is to be noted that numbering of Fc-Cε3/Cε4 (also referred to herein as Fc Cε3-Cε4) residues follows the convention of Dorrington et al, ibid.

IgE antibodies mediate anti-parasitic immune responses and the inflammatory reactions of allergy and asthma. This Example describes the solution of a crystal structure of the human IgE-Fc Cε3-Cε4 domains to 2.3 Å resolution, the coordinates of which are disclosed in Table 2 and Table 3. The IgE-Fc crystal structure reveals a novel, closed conformation for Fc domains. For example, the structure reveals a large rearrangement of the N-terminal Cε3 domains when compared to related IgG-Fc structures and to the IgE-Fc bound to its high affinity receptor, FcεRI. The IgE-Fc adopts a more compact, closed configuration that places the two Cε3 domains in close proximity, decreases the size of the interdomain cavity and obscures part of the FcεRI-binding site. Unique structural features of the Cε3-Cε4 interdomain interfaces are identified that may enable this conformational flexibility. Fc domain flexibility may allow IgE to form optimal interactions with both of its receptors, FcεRI and FcεRII. The structure of the IgE-Fc suggests new strategies for anti-allergy treatments including the design of molecules that act allosterically to block receptor binding.

A. Background

The functional diversity of the antibody repertoire involves both the creation of antigen-specific binding sites and the coupling of these specific binding sites to different effector mechanisms of the immune system. Within the antibody, these two functional roles are found on separable parts of the protein, the Fab and Fc regions. Two antigen-binding sites are contained within the Fab regions of antibodies, which are covalently linked through the antibody heavy chain to Fc effector domains (Harris et al., 1999; Padlan, 1994). The Fc domains provide specificity for the activation of downstream effector functions and are derived solely from constant domains of the antibody heavy chain. Isotype switching during B cell development produces immunoglobulins with identical antigenic specificity connected to different heavy chain constant regions that fall into five major classes or isotypes: IgA, IgD, IgE, IgG and IgM. Different Fc isotypes interact with distinct sets of cellular receptors or soluble proteins to initiate specific defense mechanisms. Effector mechanisms are adapted for specific pathogens, for the physical location of an infection and for different stages of the immune response. Fc-associated effector mechanisms include phagocytosis, the initiation of cellular cytotoxicity and inflammation pathways, the activation of complement and the feedback regulation of antibody production (Daeron, 1997; Kinet, 1999; Ravetch and Clynes, 1998).

IgE antibodies interact through their Fc domains with two cellular receptors of the immune system, FcεRI and FcεRII (CD23). IgE antibodies bind to the high affinity receptor, FcεRI, on the surface of mast cells, basophils and eosinophils (Kinet, 1999; Metzger, 1992). Binding of polyvalent antigen by the receptor-bound IgE causes receptor aggregation, triggering cellular activation. On mast cells this leads to the release of histamine, inflammatory mediators and vasodilators. Mast cell reactions to environmental allergens are associated with the pathologies of allergy, asthma and anaphylaxis (Turner and Kinet, 1999). Activation of eosinophils by FcεRI provides defense mechanisms against parasitic infection (Gounni et al., 1994; Kinet, 1999), while FcεRI on dendritic cells can deliver IgE-bound antigen into the MHC class II antigen-presentation pathway (Maurer et al., 1998). IgE antibodies also interact with a lower affinity receptor, FcεRII, involved in antigen presentation, cellular cytotoxicity and the regulation of IgE production (Sutton and Gould, 1993). While FcεRI is homologous to a family of antibody receptors specific for IgE, IgG and IgA antibodies, FcεRII belongs to a different structural class of proteins and is uniquely associated with the IgE system.

IgE is the target of recent therapeutic approaches for asthma using humanized anti-IgE monoclonal antibodies (Chang, 2000; Jardieu and Fick, 1999). Antibodies directed against the IgE-Fc block receptor binding, leading to a decrease in receptor activation and expression levels and triggering a decrease in IgE serum levels. Structural studies of the IgE-Fc may provide new routes to improving anti-IgE therapies and to designing inhibitors for the treatment of a wide variety of atopic diseases.

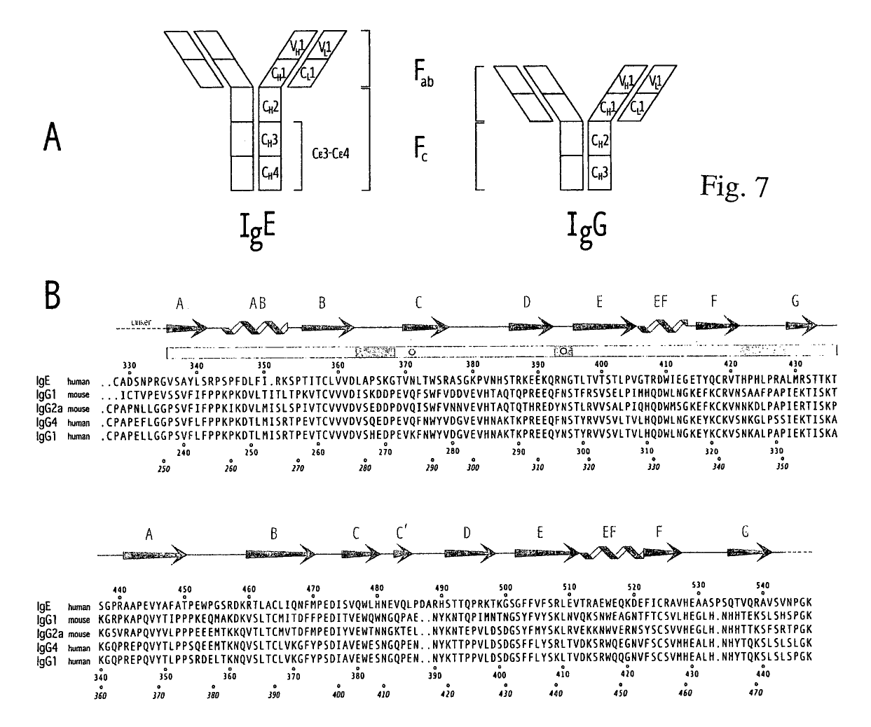

IgE contains two antibody light chains associated with two heavy chains of the ε isotype. The three C-terminal constant domains of the heavy chain (Cε2, Cε3 and Cε4) dimerize to form the Fc effector domains. Compared to IgG, IgE antibodies have an additional constant domain, Cε2 (FIG. 7a). The Cε3 and Cε4 domains are homologous to the IgG-Fc Cγ2 and Cγ3 domains, respectively, with 32% sequence identity between human IgE and IgG1 (FIG. 7b). Both intact IgE and IgE-Fc fragments (Cε2-Cε4, Cε3-Cε4) bind with high affinity (K_{D} ˜10⁻⁹-10⁻¹⁰ M) to FcεRI and mutagenesis studies have implicated Cε3-domain residues in mediating this interaction (Basu et al., 1993; Henry et al., 1997; Nissim et al., 1991; Presta et al., 1994; Weetall et al., 1990), as well as the binding to FcεRII (Shi et al., 1997; Sutton and Gould, 1993). IgE-Fc Cε3-Cε4 retains binding to both FcεRI (Basu et al., 1993; Henry et al., 1997; Young et al., 1995) and FcεRII (Shi et al., 1997), suggesting a minimal construct for structural studies.

Crystallographic studies of antibody Fc domains have previously been limited to the IgG class, leaving open many questions regarding the role of sequence and structural diversity in Fc-effector functions.

B. Structure Determination

The C-terminal domains of human IgE-Fc, Cε3-Cε4 (FIG. 7), were expressed in insect cells as described in Methods. The IgE-Fc Cε3-Cε4 protein contains three potential N-linked carbohydrate attachment sites, but only two are glycosylated in vivo, N371 and N394 (Basu et al., 1993; Young et al., 1995). Characterization of the Fc carbohydrate by endoglycosidase digestion, mass spectroscopy of tryptic peptides, and mutational analysis shows that high-mannose carbohydrate is attached to N394, which is a conserved glycosylation site in Fc domains. Although both deglycosylated IgE-Fc (Basu et al., 1993) and high-mannose IgE (Granato and Neeser, 1987) retain high binding affinity for FcεRI, deglycosylated IgE-Fc has a tendency to aggregate, making it a poor candidate for crystallographic studies (Basu et al., 1993).

The IgE-Fc was purified to homogeneity and crystallized. Crystals belong to space group P42₁2 with cell dimensions a=b=105.6 Å, c=47.1 Å. The crystals contain a single IgE-Fc chain (half of the dimeric molecule) in the asymmetric unit, with the molecular dimer axis lying along a crystallographic dyad. The crystals diffract X-rays to 2.0 Å using synchrotron X-ray sources. Molecular replacement searches using a variety of IgG-Fc models were unsuccessful, as were heavy atom searches. The IgE-Fc Cε3-Cε4 structure was solved by an automated molecular replacement search using ˜12,000 distinct conformational variants of core models for the two Ig domains, systematically varying the angles relating the Cε3 and Cε4 domain models. Data collection and refinement statistics are shown in Table 7. The current R_{free} and R_{work} are 27.0% and 24.2%, respectively, to 2.3 Å resolution. There is no density for the ten amino-terminal residues of the protein (including the interchain disulfide) and the four C-terminal residues. In addition, the density for the Cε4 AB loop is poor.

TABLE 7
Data Collection and Refinement
Data Collection Statistics

| Data Set | wtcu3 (pH 6.5) | wtcu1 (pH 7.5) |
|---|---|---|
| Source | APS DND 5-ID | SSRL 7-1 |
| Wavelength | 0.906 | 1.08 |
| (Å) | | |
| Resolution | 30.0-2.30 | 30.0-2.60 |
| (Å) | (2.38-2.30)^{^{†}} | (2.69-2.60)^{†} |
| Completeness | 99.8% (99.5%)^{†} | 99.9% (99.0%)^{†} |
| Unique | 12,340 (106,855) | 8,586 (83,604) |
| reflections | | |
| (Total) | | |
| Average | 8.7 (>7.1)^{†} | 9.7 (>7.4)^{†} |
| redundancy | | |
| <I/σ_{I}> | 33.9 (10.6)^{†} | 13.9 (8.6)^{†} |
| R_{merge} | 5.4% (25.7%)^{†} | 6.6% (24.5%)^{†} |
| Refinement (wtcu3): | | |
| Reflections, | 11053 (1269) | |
| work (free) | | |

| | Atoms | Protein | Water |
|---|---|---|---|
| R_{work}/R_{free} | (Total) | Atoms | Molecules |
| 24.2/27.0 | 1,763 | 1,618 | 145 |

| | |
|---|---|
| | RMS Deviations |
| Average B factor | from Ideality |

| | | | |
|---|---|---|---|
| Protein | Water | Bond angles | Bond lengths |
| 51.8 Å² | 59.0 Å² | 1.77° | 0.007 Å |

Ramachandran (φ, ψ)

| | | | |
|---|---|---|---|
| Favored | Allowed | Generous | Disallowed |
| 87.3% | 12.2% | 0.0% | 0.6% |

^{†}Values for the highest resolution shell are shown in parentheses
R_{merge} = Σ [symbol] Iᵢ − <I> [symbol] /Σ [symbol] I [symbol] , where Iᵢ is the intensity of an individual reflection and <I> is the average intensity of that reflection.
R_{work/free} = Σ| [symbol] Fₚ [symbol] − [symbol] F_{c} [symbol] |/Σ [symbol] Fₚ [symbol] , where F_{c} is the calculated and Fₚ is the observed structure factor amplitude. R_{work} and R_{free} were calculated using the working set and test set reflections, respectively.

C. Description of the IgE Structure

The Cε3 and Cε4 domains of IgE belong to the C1 set of Ig constant domains (Murzin et al., 1995). While the IgE Cε3 and Cε4 domains are individually similar to IgG Cγ2 and Cγ3 domains, respectively, a structure-based sequence alignment of the IgE-Fc and IgG-Fcs reveals several changes in secondary structure (FIG. 7b). Compared to IgE, the IgG contains a single residue insertion (1253 in IgG1) that forms a bulge just after the Cγ2 AB helix. The IgE Cε3 domain lacks a C′ strand and the Cε4 domain has a poorly ordered AB loop in place of the AB helix in Cγ3. Two prolines (P381 and P454) may contribute to the disruption of these secondary structures by altering hydrogen bond capabilities.

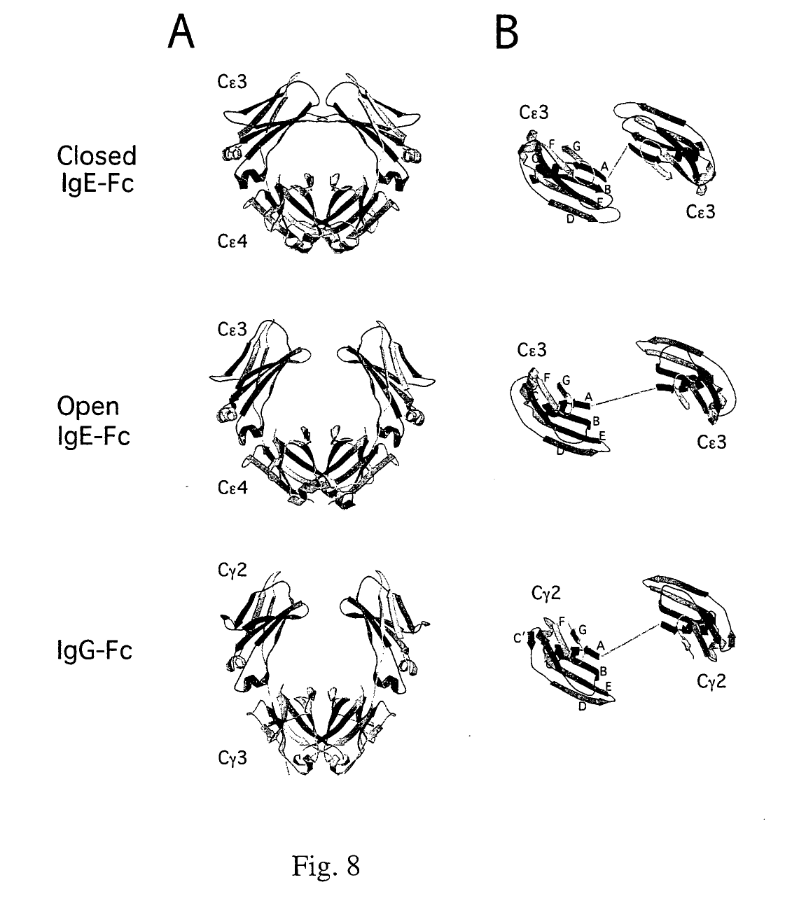

A ribbon diagram of the IgE-Fc is shown in FIG. 8. As in IgG-Fcs, the upper domains (Cε3) of the IgE-Fc do not form any direct protein:protein contacts. The conserved carbohydrate attachment site (N394) faces the cavity between the Cε3 domains. While the observed electron density is consistent with glycosylation at this site, the poor quality of the density precludes modeling of carbohydrate. Inclusion of carbohydrate residues in the model did not decrease the R_{free} or improve the electron density maps. However, the electron density suggests that carbohydrate residues contact each other near the Fc dimer axis, forming the bottom of a narrow cleft between the Cε3 domains. The Cε4 domains form extensive contacts across the dimer interface, burying ˜1,860 Å². Fourteen atomic contacts (<4 Å) formed between the Cε3 and Cε4 domains of a single chain bury 872 Å², and so bury a total of 1,744 Å² in the dimer.

No electron density for residues N-terminal to V336 (the Cε2-Cε3 linker region) is observed, despite the formation of the interchain disulfide in this region. These Cε2-Cε3 linker residues are ordered in the complex with FcεRI (Garman et al., 2000) and several of these residues interact with the receptor. While the absence of the Cε2 domains may contribute to the disorder of the Cε2-Cε3 linker in the free Fc, the asymmetric binding of linker residues to FcεRI suggests that flexibility is functionally important.

D. The IgE-Fc Adopts a Novel Conformation

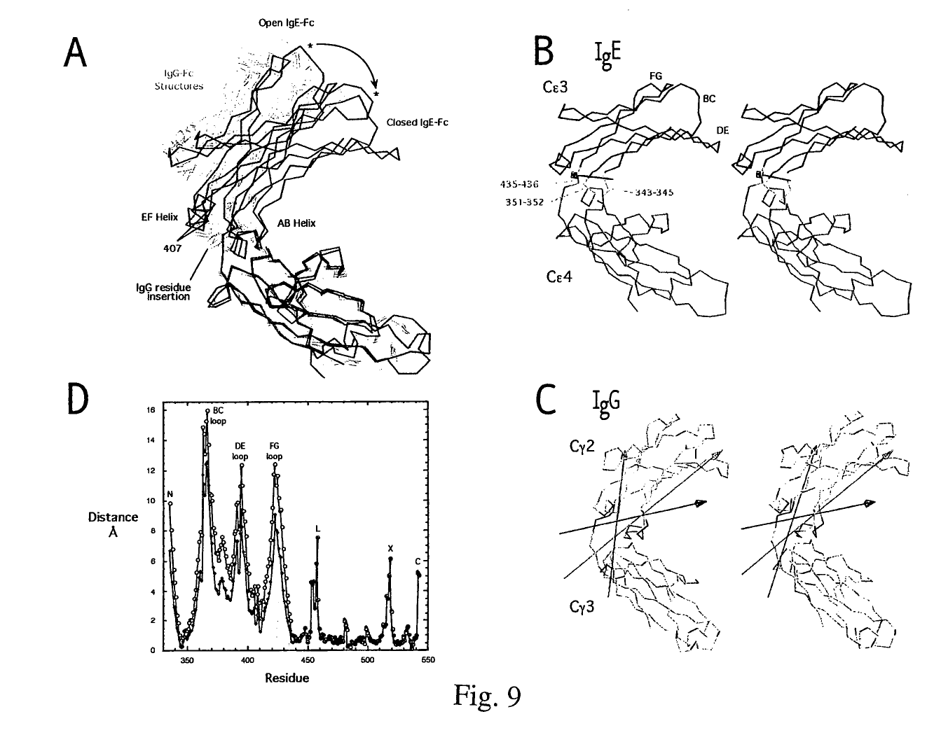

The crystal structure of the IgE-Fc Cε3-Cε4 domains reveals a novel, closed conformation for antibody effector domains (FIG. 8). In the free IgE-Fc, the Cε3-Cε4 interdomain angle is more acute than that observed between homologous IgG-Fc domains (Deisenhofer et al., 1976; Harris et al., 1999) or in the FcεRI-bound IgE-Fc (open conformation, Garman et al., 2000). Both the relative dispositions of the two Cε3 domains with respect to each other and to the Cε4 domains is altered. In the closed structure, the IgE-Fc Cε3 domains are closer together and slightly rotated with respect to each other. A top view of the Cε3 and Cγ2 domains illustrates differences in the interdomain gap (FIG. 8b). In the IgE-Fc, the distance between the first residue of the Cε3 A strands is only 13 Å. The distance increases to 23 Å in the receptor-bound IgE-Fc, which is similar to the 22 Å observed between the Cγ2 domains in IgG2a-Fc (Harris et al., 1997). The Cε3 domains not only approach each other more closely, but they also lie closer to the Cε4 domains. For example, the top of the Cε3 domain (residue T396 in DE loop) is 23 Å from the top of the Cε4 domain (residue S491). The distance between the corresponding residues in IgG2a is 33 Å, and in the receptor-bound IgE-Fc (open form), the distance is 31 Å. Thus, in the change between the open and closed forms, the top of each Cε3 domain moves 10 Å towards the other Cε3 domain across the dimer axis and 8 Å towards the Cε4 domain of the same chain. The closer approach of the upper domains of IgE (Cε3) to the lower domains (Cε4) decreases the overall height of the IgE-Fc by ˜7 Å compared to the IgG-Fc. The IgE-Fc conformational change is much greater than any differences observed among IgG-Fc crystal structures. Six crystal structures of the IgG-Fc provide nine different observations of a single chain of the IgG-Fc (in three structures, the two chains are constrained by crystallographic symmetry to be identical). These nine IgG-Fc chains, aligned via their Cγ3 domains, reveal IgG-Fc conformational variability as a family of Cγ2 positions (FIG. 9a). In the closed structure, the IgE Cε3 domain lies far outside the range of observed IgG-Fc conformations. When bound to FcεRI, the angle between the Cε3 and Cε4 domains increases and the Cε3 domains approach the observed positions for IgG Cγ2 domains. Some of the structural variation in the IgG-Fcs may be attributable to sequence differences. While the human IgG structures share ˜95% sequence identity and the mouse structures have ˜67% identity, the largest difference in IgG Cγ2 positions occurs between the human and mouse structures which share ˜64% identity (Harris et al., 1999). However, the largest conformational change occurs between the open and closed forms of the IgE-Fc, which are identical in sequence, demonstrating the inherent flexibility of the IgE-Fc.

E. Analysis of IgE- and IgG-Fc Conformational Flexibility

The IgE-Fc conformational change can be described by an axis relating the two Ig domains in the open and closed conformations. The program DynDom (Hayward and Berendsen, 1998) defines a rotation of ˜13° and a translation of 1 Å about this axis (arrow in FIG. 9b). Surprisingly, the axis does not lie in the Cε3-Cε4 linker region (436-440) but rather within the Cε3 domain itself, near the Cε3-Cε4 domain interface. Hinge residues that mediate the conformational change lie at both ends of the Cε3 AB helix (residues 343-345 and 351-352) and adjacent to the Cε3-Cε4 linker (residues 435-436). None of the observed IgG-Fc structures exhibit such a large degree of flexibility. Three IgG-Fc structures have been solved in which the two Cγ2 domains of the same Fc exhibit different orientations with respect to their Cγ3 domains (1FC1 (Deisenhofer, 1981), 1IGY (Harris et al., 1998), and 1IGT (Harris et al., 1997)). Since the structural variation occurs within the same Fc, differences due to sequence variation are eliminated. For each structure, DynDom analysis identifies an axis near the Cγ2-Cγ3 interface that describes a motion of 6-7° between the two conformers (FIG. 9c). The orientations of the axes are different from each other and from that of the IgE-Fc, and they describe distinct movements (e.g. side-to-side) of Cγ2. However, none of the IgG motions match the open-to-closed conformational change seen in the IgE-Fc. The different location of the hinge axes and the much smaller range of motion displayed by the IgG-Fc suggest that the flexibility of Ig domains involves multiple factors.

The change in Cα coordinates between the closed and open conformations of the IgE-Fc is plotted in FIG. 9d. The changes are slightly different for the two Fc chains that bind asymmetrically to the FcεRI (Garman et al., 2000). The Cε3 AB helix (344-352) and the interdomain linker (436-440) remain relatively fixed with respect to the Cε4 domain, while the Cε3 EF helix residues (406-413) show Cα movements of up to 4 Å (FIG. 9a). Positional changes become greater further from the hinge, with the greatest displacement of Cε3 residues observed at the top of the Fc in the BC (363-368), DE (393-395) and FG (422-428) loops that bind to FcεRI (FIG. 9b). Residues in these loops move 7-16 Å between the open and closed conformations (FIG. 9c). Large differences are also observed in the A-strand adjacent to the Cε2-Cε3 linker.

F. IgE-Fc Carbohydrate

In both IgE and IgG, a conserved carbohydrate attachment site faces the cavity between the upper domains (Cε3 and Cγ2 respectively). Carbohydrate residues have not been included in the structure, but partial electron density for ˜5 carbohydrate moieties can be observed at the conserved N394 site, branching after the core (-GlcNAc₂Man) into two arms. As in IgG, electron density for carbohydrate lies along the inner face of the protein, shielding hydrophobic residues from solvent (IgE residues Y339, L359, V361). The carbohydrate is not sequestered from solvent, however. In IgE, the carbohydrate attached to N394 can be removed by endoglycosidases under native conditions, suggesting that this region is at least transiently accessible in solution (Basu et al., 1993).

Carbohydrate is not required for high affinity binding to FcεRI, suggesting that it does not affect the conformation of the IgE-Fc significantly. IgA glycosylation is similarly not required for Fc-receptor binding (Mattu et al., 1998). In contrast, the presence of carbohydrate at a conserved N-linked attachment site in IgG (N297 in IgG1) is critical for maintaining Fc receptor-binding activities (Jefferis et al., 1998). Core glycosylation (-GlcNAc₂Man₃) of IgG, produced in mammalian, yeast and insect cells, is likely sufficient for this carbohydrate function (Jefferis et al., 1998). Functional and biophysical studies of IgG indicate that the carbohydrate moiety has only a limited and local effect on the Fc structure (Jefferis et al., 1998). A comparison of glycosylated and aglycosylated IgG-Fc with a panel of monoclonal antibodies showed no detectable epitope differences, suggesting that global structural changes were not occurring (Walker et al., 1989). ¹H-NMR has been used to study the influence of glycosylation on the structure of IgG-Fc. Histidine resonances were monitored in glycosylated and non-glycosylated IgG-Fc (Lund et al., 1990; Matsuda et al., 1990). Of the five histidines monitored, only one near the conserved glycosylation site (H268 in the Cγ2 BC loop), reported any change in local environment. Histidines at the Cγ2-Cγ3 domain interface did not detect any structural differences. Based on the IgE-Fc:FcεRI crystal structure, the Cγ2 BC loop and DE loop containing the conserved glycosylation site are predicted to participate directly in FcγR interactions (Garman et al., 2000). Local structural changes in these loops could affect receptor binding.

G. Structural Changes at the Interdomain Interface

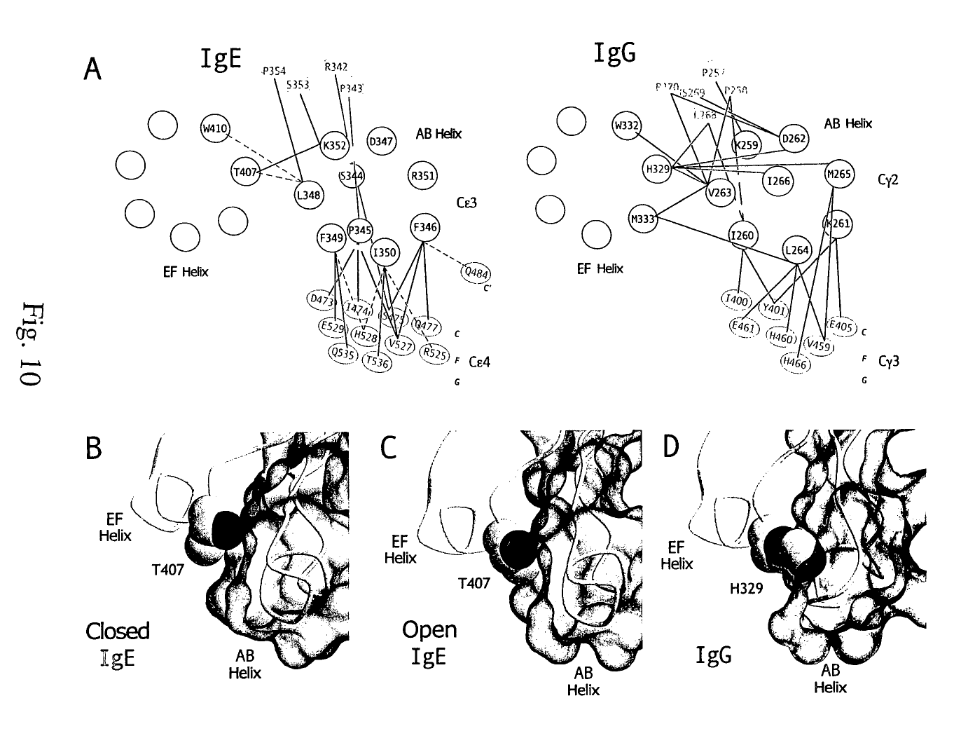

The interdomain interfaces of both IgG-Fc (Cγ2-Cγ3) and IgE-Fc (Cε3-Cε4) are important for Fc function, and structural differences in the interface may influence Fc domain flexibility. Four proteins bind to this region in IgG: neonatal Fc receptor (Burmeister et al., 1994), rheumatoid factor (Corper et al., 1997), Protein A (Deisenhofer, 1981), and Protein G (Sauer-Eriksson et al., 1995). Direct binding of proteins to this region in IgE has not been demonstrated. The binding site for FcεRII has been broadly mapped to the outer surface of Cε3 (Shi et al., 1997; Sutton and Gould, 1993), while the FcεRI binding site is distal to this interface and encompasses the Cε3 BC, DE, and FG loops as well as the Cε2-Cε3 linker (Garman et al., 2000; Henry et al., 1997; Presta et al., 1994). However, despite the fact that residues at the Cε3-Cε4 domain interface do not form direct contacts to the FcεRI, mutations in this region can have a profound effect on FcεRI binding. For example, substitution of IgE Cε3 AB helix residues with IgG Cγ2 AB helix residues disrupts binding, as does a single amino acid mutation, F329A (Presta et al., 1994), suggesting that interactions at the Cε3-Cε4 domain interface are important in maintaining a functional Fc. The AB helix (in Cε3 or Cγ2) mediates the majority of atomic contacts (atoms within 4 Å) between the Fc domains in both IgE and IgG (FIG. 7a). The AB helix contacts adjacent residues and residues in the EF helix of the upper domain. The AB helix also contacts residues in the C, F and G strands and FG loop of the lower Ig domain (Cε4 or Cγ3). Despite the central role of the AB helix in mediating interdomain contacts, AB helix residues are not conserved between IgE and IgG. Only one residue of the helix (εD347, γD262) is invariant (FIG. 7b). In addition, most of the residues that contact the AB helix are not conserved between IgE and IgG. The pattern of contacts made by AB helix residues is different in IgE and IgG. In IgE, most of the contacts made by the AB helix are to residues of the lower (Cε4) domain (15/21 in the open conformation, FIG. 10a). Only one contact is made to the EF helix in the closed form and two additional contacts are formed in the open conformation (dashed lines). In IgG, the majority of the AB helix contacts (12/21) are to other residues within the same Cγ2 domain, with nine contacts to the lower Cγ3 domain. The Cγ2 AB helix forms extensive contacts to the EF helix. Two residues in particular, V263 and H329, form a network of nine contacts within the Cγ2 domain. There are two striking structural features unique to the IgG interdomain interface (FIGS. 9a, 10d). A single residue insertion after the IgG Cγ2 AB helix, isoleucine 266, forms a distinct bulge at the end of the helix. This isoleucine, together with adjacent residues, forms part of a shallow pocket on the surface of the IgG (FIG. 10d). The second difference in IgG is the presence of a conserved histidine (H329) on the EF helix facing the AB helix. This histidine is completely conserved in IgGs across species and subtypes but is not found in other Ig isotypes. Histidine 329 forms five atomic contacts to the pocket formed by 1266 and neighboring residues (FIG. 10d). The contacts made by H329 are maintained in all IgG-Fc structures, including a highly distorted Fab-Fc hinge-deleted IgG in which the AB helix no longer contacts the lower Ig domain and has shifted away from the Cγ2-Cγ3 domain interface (Guddat et al., 1993). In non-glycosylated IgG-Fc, the ¹H resonances of IgG H329 do not change (Lund et al., 1990; Matsuda et al., 1990), suggesting the preservation of these interactions.

In contrast, the corresponding residue in IgE, T407, makes two contacts to the AB helix in the open form (FIGS. 10a, 10c) and makes only one contact and moves away from the AB helix in the closed form (FIGS. 9a, 10a-c). In rat and mouse IgE sequences T407 is replaced by alanine, suggesting that the conservation of these side chain interactions is not important.

The extensive contacts formed by the IgG AB helix to other Cγ2 domain residues and the close packing of EF helix residue H329 to the AB helix distinguish the IgG Cγ2-Cγ3 interface. In IgG, the AB helix is more closely associated with the upper (Cγ2) domain than the lower (Cγ3) domain. In contrast, the IgE interface is characterized by extensive interactions of the Cε3 AB helix residues with the lower Cε4 domain residues (FIG. 10a), and contacts with the EF helix are limited. In both IgE Cε3 and IgG Cγ2, the A and B strands separate and do not form hydrogen bonds on either side of the AB helix, allowing for some flexibility in the positioning of the AB helix. However, the flexibility may be limited in IgG by the extensive interactions of the Cγ2 AB and EF helices. In IgE, the limited contacts made between the Cε3 AB and EF helices may allow the helices to move independently.

H. Effect of the Conformational Change on the FcεRI Binding Site

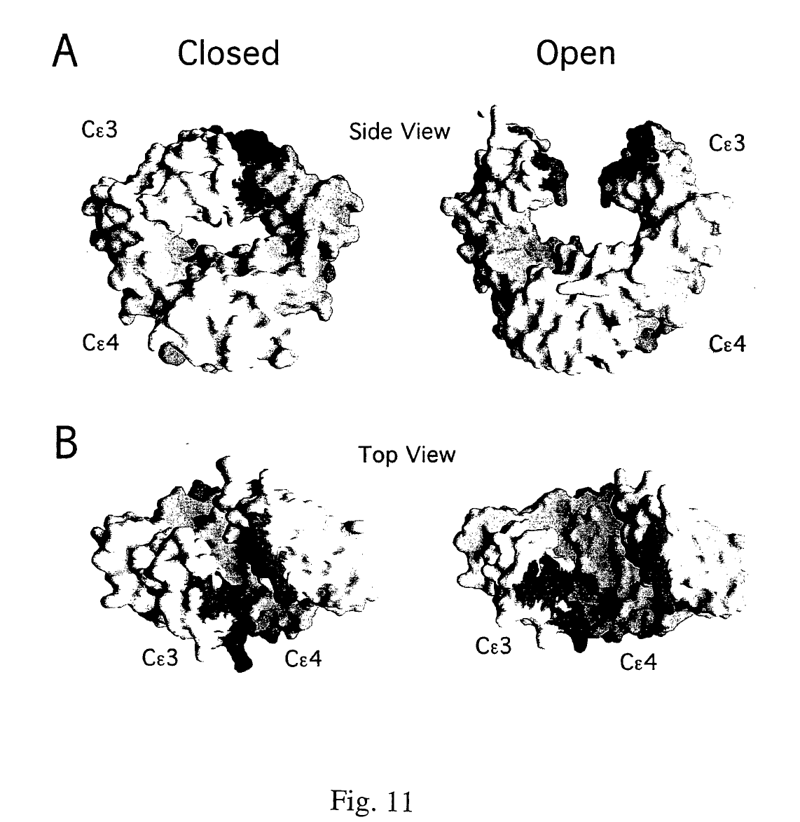

The large conformational change of the IgE-Fc structure reorients loops in the Cε3 domains that interact with the high affinity receptor, FcεRI. The large movement of the FcεRI-binding loops suggests that they would be poorly positioned in the closed IgE-Fc structure to interact with the receptor. FIG. 11 shows a molecular surface representation of the open and closed Fc structures, with the receptor-binding residues highlighted in magenta. In the open form, the receptor-binding loops are exposed and the binding residues display a large concave surface that is available to interact with FcεRI. In the closed form, these loops are partially obscured and point towards each other across the IgE-Fc dyad axis, leaving only a narrow gap between the two Cε3 domains that cannot accommodate the binding of the receptor. While the Cε3 BC, DE and FG loops are largely inaccessible in the closed conformation, the disordered Cε2-Cε3 linker residues N-terminal to V336 could form an initial interaction with the receptor even in the closed IgE-Fc structure. Binding of the receptor to linker residues might shift the conformation of the Fc towards the open form, exposing the binding loops.

I. Structural Basis for the IgE-Fc Conformational Flexibility

The IgE-Fc structure reveals an unprecedented conformation for antibody effector domains with implications for Fc-receptor binding and therapeutic intervention in human disease. The structure of the closed IgE-Fc suggests that the effector domains of antibody isotypes may have evolved structural characteristics that are associated with isotype-specific biological functions. Structural features that could influence the flexibility of the IgE-Fc include the location and packing of hinge residues and the specific interactions at the Cε3-Cε4 domain interface, such as the position and contacts of the Cε3 AB helix. Other factors that could potentially effect a change in conformation have been considered, such as the specific crystal-packing environment, the presence of high-mannose instead of complex carbohydrate, or the lack of the Cε2 domains. The present invention also includes the solution of a second crystal form of the IgE-Fc containing two IgE-Fc molecules in the asymmetric unit, both in the closed form. These five IgE-Fc chains all adopt a similar conformation, indicating that the closed conformation is not dictated by specific crystal-packing forces.

It remains to be established whether different carbohydrate structures at the conserved attachment site could influence the extent of the observed IgE-Fc conformational change. Biochemical studies of IgG suggest a limited structural role for the conserved carbohydrate in maintaining the overall three-dimensional arrangement of Fc domains, as discussed above. While functional studies of the IgE-Fc (FcεRIα binding) argue against a significant role for the conserved carbohydrate, structural studies of different IgE-Fc glycoforms may resolve this issue.

Biochemical and biophysical studies indicate that the IgE-Fc Cε2 domains form a separate structural unit from the Cε3-Cε4 structure solved here. The Cε2-Cε3 linker is susceptible to proteolytic digestion (Perez-Montfort and Metzger, 1982) and adopts an asymmetric conformation upon binding FcεRI (Garman et al., 2000), suggesting that it is accessible and flexible. The presence or absence of the Cε2 domains in the IgE-Fc does not significantly alter the binding constants or thermodynamic parameters (ΔG°, ΔH°, ΔS°, and ΔCp°) of FcεRI binding (Keown et al., 1998). Therefore, the mode of binding to the receptor is likely to be similar for intact IgE-Fc and IgE-Fc Cε3-Cε4. Together, these results suggest that the Cε2 domains have little influence on the structure or conformation of the Cε3 domains.

Structural characteristics of the IgE Cε3-Cε4 domain interface, as compared to the IgG Cγ2-Cγ3 domain interface, likely enable the conformational flexibility of the IgE-Fc. The AB helix of the first domain (Cε3 or Cγ2) mediates most of the interdomain contacts in the Fc structure and is not conserved in sequence across the five different antibody classes. Packing contacts of the AB helix with the two Fc Ig domains may differ significantly across antibody isotypes, potentially influencing Fc conformation, flexibility and function. The range of conformational flexibility of the Fc domains of different antibody classes could be linked to the evolution of isotype-specific effector functions. The more limited flexibility of IgG structures may reflect similarities in the structural requirements for FcγR and complement (C1q) interactions.

Other experimental evidence has suggested that IgE adopts a bent configuration in solution and that conformational changes may occur upon binding to FcεRI. The design and interpretation of these experiments could not have anticipated the specific IgE-Fc conformational change of the present invention. Binding of IgE-Fc to FcεRI (Keown et al., 1998) is characterized by a relatively large change in heat capacity (ΔCp°=−815 cal/mol K), which could be in part be caused by IgE-Fc conformational changes. In contrast, binding of IgG-Fc to its homologous low affinity receptor, FcγRIII, exhibits a smaller change in heat capacity (ΔCp°=−360 cal/mol K). Fluorescence energy transfer experiments have shown that the average distance between the N- and C- termini of the IgE is only ˜70 Å, a distance that is possible only if the IgE bends significantly out of the plane of the typical antibody Y- or T-shape (Zheng et al., 1991). Neutron scattering studies have shown that the intact IgE-Fc (Cε2-Cε4) has a significantly more compact shape than a linear arrangement of the domains would allow (Beavil et al., 1995), suggesting that a bend occurs within the IgE-Fc region. The IgE-Fc crystal structure supports the interpretation of bending of the intact IgE at the Cε2-Cε3 linker region, and may provide a better model for the analysis of the neutron scattering data. Experimental tests of IgE flexibility can now be developed based on the structure.

J. Biological and Therapeutic Implications for IgE-Fc Conformational Flexibility

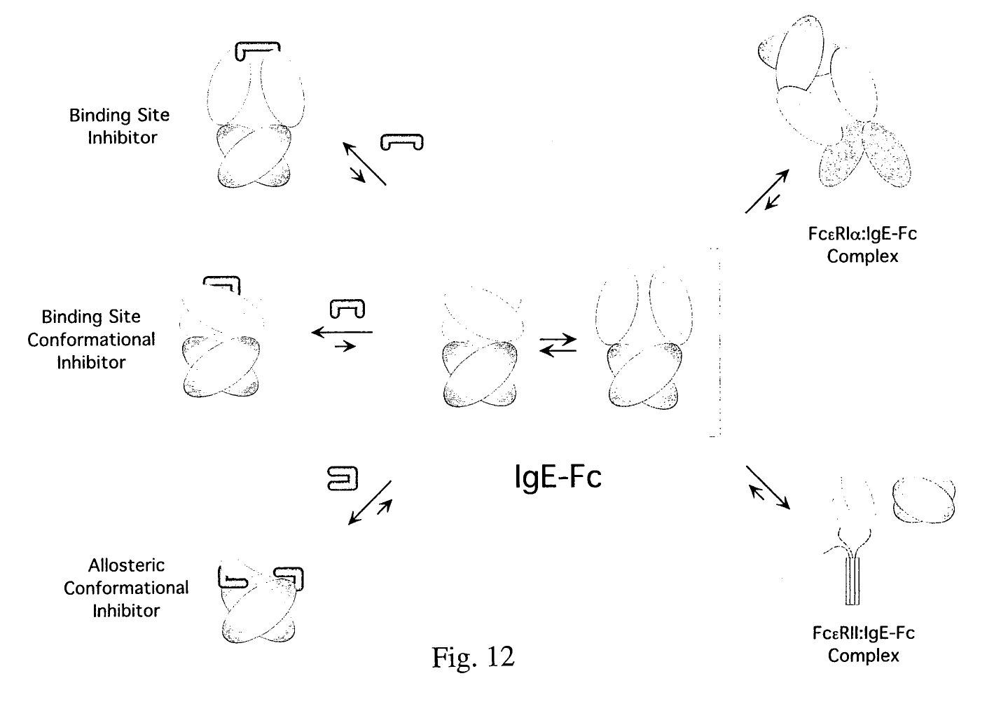

Conformational flexibility in the IgE-Fc may be important for unique aspects of IgE biological function. IgE-Fc flexibility may allow induced-fit interactions with FcεRI, contributing to the high affinity binding, and may be important for interactions with the low affinity IgE receptor, FcεRII (FIG. 12). FcεRII is a trimeric C-type lectin that is thought to interact with the IgE-Fc through two of its three lectin domains (Shi et al., 1997). IgG antibodies do not have a corresponding lectin-like receptor, suggesting that the conformational flexibility of the IgE-Fc may be important for this unique antibody-receptor interaction.

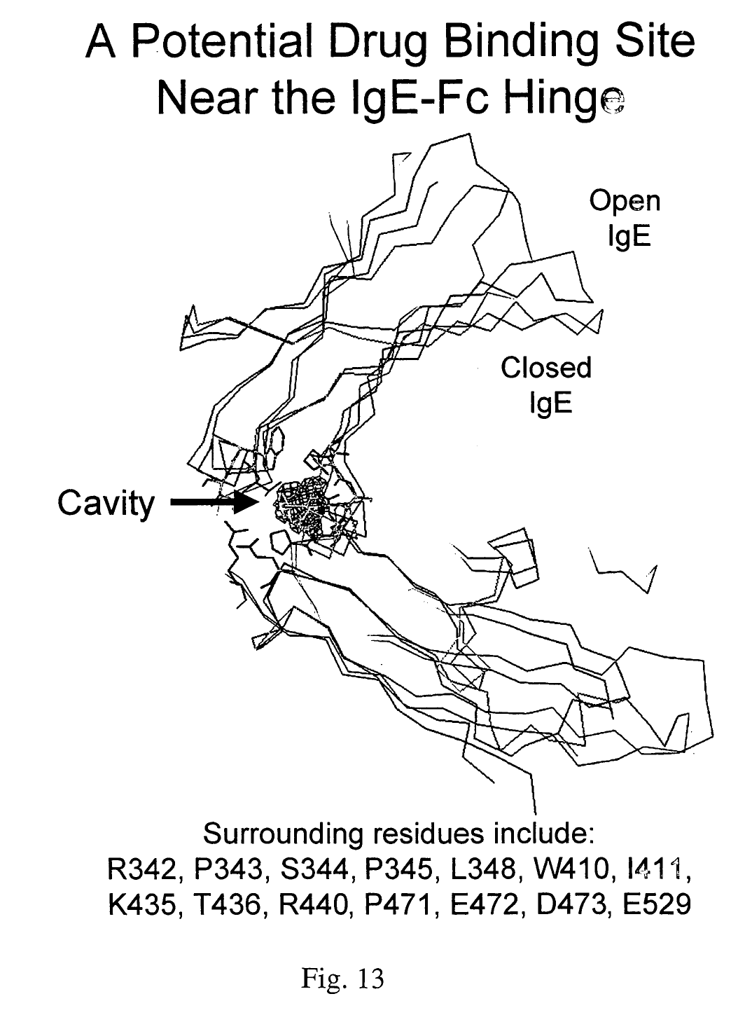

The existence of a closed conformation for the IgE-Fc and the demonstration that the open form binds to the high affinity receptor (Garman et al., 2000) suggest new strategies for the design of inhibitors of the IgE:FcεRI interaction (FIG. 12). Monoclonal antibodies that bind the IgE-Fc and block interactions with FcεRI have demonstrated the therapeutic potential of this approach for the treatment of allergy and asthma (Chang, 2000; Jardieu and Fick, 1999). Stabilization of the closed IgE-Fc conformation by the binding of molecules, either at directly competitive or indirect allosteric sites, could block receptor association, leading to a new class of therapeutic inhibitors for the treatment of IgE-mediated allergic diseases. The IgE Cε3-Cε4 domain interface may provide a target for the binding of small in vitro-selected ligands, as shown for the IgG-Fc (DeLano et al., 2000), that have the potential to act as allosteric inhibitors of receptor binding (FIG. 13). The closed conformation of the IgE-Fc provides the foundation for exploring new routes to alleviating atopic disease and exploring the functional role of Fc domain flexibility in biological effector mechanisms.

K. Methods

1. Expression and Purification of the Human IgE-Fc.

The expression, purification and characterization of the IgE-Fc from insect cells was performed as described in Example 1.

2. Crystallization and Treatment of Crystals

Purified IgE-Fc was concentrated to 10 mg/ml in 10 mM Tris, pH 8.0, using an ε₂₈₀ₙₘ of 1.32 cm⁻¹ (mg/ml)⁻¹. Crystals were grown in hanging drops using the vapor diffusion method by mixing 0.5 μl protein and 0.5 μl of precipitant (25 mM sodium acetate, pH 4.6, 33% polyethylene glycol [PEG] 4000). Crystals grew at 22° C. in 1-3 days and were sensitive to small changes in salt or PEG concentration and temperature. Crystals were harvested into 25 mM sodium acetate, pH 4.6, 37% PEG 4000, transferred briefly (<30 s) to cryoprotectant solution (harvest solution plus 15% (v/v) ethylene glycol) and cooled rapidly in liquid nitrogen. Crystals belong to the space group P42₁2 (a=b=105.6 Å, c=47.1 Å) and contain one Cε3-Cε4 chain per asymmetric unit. Crystals were transferred serially to higher pH harvest solutions to facilitate metal binding and redox chemistry. Heavy atom screening (˜100 conditions) with a wide range of compounds (27), concentrations (0.1-20 mM) and pH ranges (4.6-8.5) did not yield an isomorphous or anomalous derivative. However, mercury- or platinum-treated crystals diffracted better than native crystals. Based on these observations, crystals were treated with 1 mM copper (II) chloride prior to cooling and data collection. Native crystals diffracted to ˜2.8 Å resolution using a synchrotron source and displayed strong anisotropy and, occasionally, split lattices. Copper-treated crystals diffracted to at least 2.0 Å resolution, with little or no anisotropy. We and others (Basu et al., 1993) have observed that a small fraction of the IgE-Fc does not form the interchain disulfide. The copper II may have oxidized the remaining free cysteines to the disulfide.

3. Data Collection, Molecular Replacement and Refinement.

Data sets were collected at −160° C. from copper-treated crystals at SSRL beamline 7-1 (wtcul) using a Mar300 imaging plate system and at the Advanced Photon Source DND-CAT 5ID beamline (wtcu3) using a MarCCD detector. The data were processed and integrated using the HKL suite of programs (Otwinowski and Minor, 1997). Initial molecular replacement (MR) searches with AMoRe (Collaborative Computational Project, 1994), CNS/XPLOR (Brünger et al., 1998) and EPMR (Kissinger et al., 1999) failed, using a variety of models of IgE based on IgG-Fc structures, including individual Ig domains and a composite model incorporating seven IgG structures. A systematic exploration of the bend, twist and rotation angles of Cε3 relative to Cε4 was then undertaken. A model was constructed from the Cγ2-Cγ3 domains derived from an intact IgG structure (murine IgG2a, PDB entry 1IGT (Harris et al., 1997)), by truncating loops and non-homologous side-chains, resulting in a 144 residue model for the 222 residue IgE-Fc. The Cγ2-Cγ3 linker region of the model was placed at the origin, with Cγ2 and Cγ3 oriented to allow bending to occur about the Z-axis. Rotations around X, Y and Z were applied to the Cγ2 domain (3° steps), while leaving the Cγ3 fixed. Approximately 12,000 models were generated automatically with the program lsqkab (Collaborative Computational Project, 1994) and used in complete AMoRe (Collaborative Computational Project, 1994) searches with the 15-4 Å data from crystal wtcul (Table 7). The models covered an angular range of −30 to +40 degrees around the starting IgG2a structure. A single solution, ˜17° from the starting structure, was found and improved by a local search using 1.0° rotational increments. The finer search yielded a solution with a correlation coefficient of 38% and an R_{factor} of 48.9%. Cycles of model building into simulated-annealing composite-omit electron density maps and refinement were continued with the higher resolution data from crystal wtcu3 using the programs O (Jones et al., 1991) and CNS (Brünger et al., 1998). Refinement was performed against all data from 30-2.3 Å using |F|>0 and an anisotropic bulk solvent correction. Only refinement steps that decreased the R_{free} were accepted. The model includes residues 336-543 and lacks 10 N-terminal and 4 C-terminal residues present in the construct. The receptor binding loops (Cε3 BC, DE, and FG loops) have weaker density and higher B-factors than most of the other residues. Density for the Cε4 AB loop is particularly poor, and this loop was modeled sterically. All of the residues lie within the accepted regions of the Ramachandran plot, with the exception of N481. There is good density for this residue, however, which is in a tight turn that lacks a Gly, Ala, or Ser residue. While some density was for carbohydrate was observed at the N394 site, attempts to build carbohydrate did not improve the R_{free} or the electron density maps, and so it was not included in the model. There is no electron density for the carbohydrate attached to the N371 site. The current refinement statistics are summarized in Table 7. Figures were made using the programs MOLSCRIPT (Kraulis, 1991), GRASP (Nicholls et al., 1991) and Raster 3D (Merritt and Bacon, 1997).

L. References

Basu et al. (1993), J. Biol. Chem. 268, 13118-27.

Beavil et al. (1995), Biochemistry 34, 14449-61.

Brünger et al. (1998), Acta Crystallogr. D. Biol. Crystallogr. 54, 905-21.

Burmeister et al. (1994), Nature 372, 379-83.

Chang (2000), Nat. Biotechnol. 18, 157-162.

Collaborative Computational Project, No. 4 (1994), Acta Cryst. D 50, 760-763.

Corper et al. (1997), Nat. Struct. Biol. 4, 374-81.

Daeron (1997), Annu. Rev. Immunol. 15, 203-34.

Deisenhofer (1981), Biochemistry 20, 2361-70.

Deisenhofer et al. (1976), Hoppe-Seyler's Z. Physiol. Chem. 357, 1421-34.

DeLano et al. (2000), Science 287, 1279-83.

Dorrington et al. (1978), Immunol. Rev. 41, 3-25.

Garman et al. (2000), Nature 406, 259-266

Gounni et al. (1994), Nature 367, 183-6.

Granato et al. (1987), Mol. Immunol. 24, 849-55.

Guddat et al. (1993), Proc. Natl. Acad. Sci. USA 90, 4271-5.

Harris et al. (1997), Biochemistry 36, 1581-97.

Harris et al. (1997), Adv. Immunol. 72, 191-208.

Harris et al. (1999), J. Mol. Biol. 275, 861-72.

Hayward et al. (1998), Proteins 30, 144-54.

Henry et al. (1997), Biochemistry 36, 15568-78.

Jardieu et al. (1999), Int. Arch. Allergy Immunol. 118, 112-5.

Jefferis et al.(1998), Immunol. Rev. 163, 59-76.

Jones et al.(1991), Acta Crystallogr. A 47, 110-9.

Kelly et al.(1998), J. Immunol. 161, 6696-704.

Keown et al. (1998), Biochemistry 37, 8863-9.

Kinet, J. P. (1999), Annu. Rev. Immunol. 17, 931-72.

Kissinger et al.(1999), Acta Crystallogr. D Biol. Crystallogr. 55, 484-91.

Kraulis, P. J. (1991), J. Appl. Cryst. 24, 946-950.

Lund et al. (1990), Mol. Immunol. 27, 1145-53.

Matsuda et al. (1990), Mol. Immunol. 27, 571-9.

Mattu et al. (1998), J. Biol. Chem. 273, 2260-72.

Maurer et al. (1998), J. Immunol. 161, 2731-9.

Merritt et al. (1997), Methods in Enzymology 277, 505-24.

Metzger et al. (1992), Immunol. Rev. 125, 37-48.

Murzin et al. (1995), J. Mol. Biol. 247, 536-40.

Nicholls et al. (1991), Proteins 11, 281-96.

Nissim et al. (1991), EMBO J. 10, 101-7.

Otwinowski et al. (1997), In Methods in Enzymology: Macromolecular Crystallography, part A, C. W. Carter, Jr. & R. M. Sweet, Eds., Academic Press, pp. 307-326.

Padlan, E. A. (1994), Mol. Immunol. 31, 169-217.

Perez-Montfort et al. (1982), Mol. Immunol. 19, 1113-25.

Presta et al. (1994), J. Biol. Chem. 269, 26368-73.

Ravetch et al. (1998), Immunol. 16, 421-32.

Sauer-Eriksson et al. (1995), Structure 3, 265-78.

Shi et al. (1997), Biochemistry 36, 2112-22.

Sutton et al. (1993), Nature 366, 421-8.

Turner et al. (1999), Nature 402, B24-30.

Walker et al. (1989), Biochem. J. 259, 347-53.

Weetall et al. (1990), J. Immunol. 145, 3849-54.

Young et al. (1995), Protein Eng. 8, 193-9.

Zheng et al. (1991), Biochemistry 30, 9125-32.

While the various embodiments of the present invention have been described in detail, it is apparent that modifications and adaptations of those embodiments will occur to those skilled in the art. It is to be expressly understood, however, that such modifications are adaptations are within the scope of the present invention, as set forth in the following claims.

What is claimed is:

1. A three-dimensional model selected from the group consisting of: (a) a three-dimensional model of a human IgE Fc region comprising Cε3 and Cε4 domains (Fc-Cε3/Cε4), wherein said model substantially represents the atomic coordinates specified in a Table selected from the group consisting of Table 1, Table 2 and Table 3; and (b) a three-dimensional model comprising a modification of said model of (a), wherein said modification represents an antibody Fc region that binds to a FcεRIα protein.

2. A method to produce the three-dimensional model of claim 1, wherein the three-dimensional model represents the Fc-Cε3/Cε4 region of a human IgE antibody, said method comprising representing amino acids of said region at substantially the atomic coordinates specified in a Table selected from the group consisting of Table 1, Table 2 and Table 3.

3. A method to produce a three-dimensional model of a FcεRIα binding domain other than a human FcεRIα binding domain represented by the three-dimensional model substantially representing the atomic coordinates specified in claim 1, said method comprising homology modeling.

4. An isolated crystal of a Fc-Cε3/Cε4 region of a human IgE antibody.

5. A method to produce the isolated crystal of claim 4, said method comprising vapor diffusion.

6. An isolated Fc-Cε3/Cε4 protein selected from the group consisting of: (a) a protein consisting of SEQ ID NO:2; and (b) a homologous protein that is structurally homologous to a protein of (a), wherein said homologous protein binds to a FcεRIα protein.

7. A nucleic acid molecule comprising a nucleic acid sequence that encodes said isolated Fc-Cε3/Cε4 protein of claim 6.

8. A recombinant molecule comprising the nucleic acid sequence of claim 7.

9. A recombinant virus comprising the nucleic acid sequence of claim 7.

10. A recombinant cell comprising the nucleic acid sequence of claim 7.

11. A method to produce a protein comprising culturing the recombinant cell of claim 10.

12. A method to identify a compound that inhibits the binding between an IgE antibody and a FcεRIα protein, said method comprising using a three-dimensional model of a Fc-Cε3/Cε4 region of a human IgE to identify said compound, wherein said model substantially represents the atomic coordinates specified in a Table selected from the group consisting of Table 1, Table 2 and Table 3.

13. An inhibitory compound identified in accordance with the method of claim 12.

14. A therapeutic composition comprising the inhibitory compound of claim 13.

15. A method to protect an animal from allergy, said method comprising administering to said animal the inhibitory compound of claim 13.

16. A method to improve a function of an antibody comprising a Fc-Cε3/Cε4 region, said improved function being selected from the group consisting of increased stability, increased affinity for an IgE binding domain of a FcεRIα protein, altered substrate specificity, and increased solubility, said method comprising:
(a) analyzing a three-dimensional model substantially representing the atomic coordinates specified in a Table selected from the group consisting of Table 1, Table 2 and Table 3 to identify at least one amino acid of the Fc-Cε3/Cε4 region represented by said model which if replaced by said identified amino acid(s) improves at least one of said functions of said Fc-Cε3/Cε4 region; and
(b) replacing said identified amino acid(s) to produce a mutein having at least one of said improved functions.

17. A mutein produced by the method of claim 16, wherein said mutein has an improved function compared to a Fc-Cε3/Cε4 protein comprising amino acid sequence SEQ ID NO:2, wherein said improved function is selected from the group consisting increased stability compared to the stability of a human IgE Fc region comprising amino acid sequence SEQ ID NO:2, increased affinity for a FcεRIα protein compared to the FcεRIα affinity of a human IgE Fc region comprising amino acid sequence SEQ ID NO:2, altered substrate affinity compared to the affinity for human FcεRIα of a human IgE Fc region comprising amino acid sequence SEQ ID NO:2, and increased solubility compared to the solubility of a human IgE Fc region comprising amino acid sequence SEQ ID NO:2.

18. A polypeptide selected from the group consisting of a FcεRIα binding domain, an interdomain groove between the two Cε3/Cε4 domains of said antibody Fc region, a hinge between Cε3 and Cε4 domains of said antibody Fc region, and a region of a Cε3 or Cε4 domain, the relative position of which changes by greater than 1 angstrom between closed and receptor-bound Fc-Cε3/Cε4 conformations.

19. The polypeptide of claim 18, wherein said composition is selected from the group consisting of a linker between Cε2 and Cε3, a BC loop of Cε3, a DE loop of Cε3, and a FG loop of Cε3, a loop or strand defining the interdomain groove, a AB helix of Cε3 and, a region lying above said AB helix of Cε3.

20. An isolated nucleic acid molecule encoding the polypeptide of claim 18.


SEQUENCE LISTING (7 sequences)

SEQ ID NO: 1 - DNA, 669 bp, Homo sapiens
  CDS (1)..(666)
gcggatccctgtgattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaatga

SEQ ID NO: 2 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 3 - protein, 220 aa, Homo sapiens
CADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 4 - protein, 217 aa, Mus musculus
ICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 5 - protein, 219 aa, Mus musculus
CPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - protein, 219 aa, Homo sapiens
CPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 219 aa, Homo sapiens
CPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK